(19) United States
(12) Patent Application Publication
(10) Pub. No.: US 2023/0406853 A1
(43) Pub. Date: Dec. 21, 2023

(54) COVALENT CDK2-BINDING COMPOUNDS FOR THERAPEUTIC PURPOSES

(71) Applicant: Umbra Therapeutics, Inc., Lexington, MA (US)

(72) Inventors: Guillaume Barbe, Milton, MA (US); Mark George Sauliner, Higganum, CT (US); William John Greenlee, Teaneck, NJ (US); Soumya Ray, Quincy, MA (US)

(73) Assignee: Umbra Therapeutics, Inc., Lexington, MA (US)

(21) Appl. No.: 18/242,466

(22) Filed: Sep. 5, 2023

Related U.S. Application Data
(63) Continuation of application No. PCT/US2022/019005, filed on Mar. 4, 2022.
(60) Provisional application No. 63/157,374, filed on Mar. 5, 2021.

(51) Int. Cl. C07D 471/04; A61P 35/00

(52) U.S. Cl. CPC C07D 471/04; A61P 35/00

(57) ABSTRACT

Heteroaryl sulfonyl compounds and compositions that have a CDK2 Recognition Moiety bound to an electrophile for the selective covalent modification of CDK2 to treat CDK2-mediated disorders are described.

### CROSS-REFERENCE TO RELATED APPLICATIONS

This application is continuation of International Patent Application No. PCT/US2022/019005, filed in the U.S. Receiving Office on Mar. 4, 2022, which claims the benefit of U.S. Provisional Application No. 63/157,374, filed on Mar. 5, 2021. The entirety of each of these applications is hereby incorporated by reference for all purposes.

### FIELD OF THE INVENTION

This invention provides heteroaryl sulfonyl compounds, compositions, and methods of use thereof, that include a CDK2 Recognition Moiety for the selective covalent modification of CDK2 to treat CDK2-mediated disorders, typically through tyrosine or lysine.

### BACKGROUND OF THE INVENTION

Most cells in the body are terminally differentiated with protective mechanisms to prevent cellular proliferation. A small subset of cells undergo cellular proliferation mainly to replenish tissue or blood components, such as hematopoietic cells and their progeny. The body maintains a 20 careful balance between terminal differentiation and cellular proliferation.

Normal cell proliferation, referred to as cell division or the cell cycle, is divided into four phases. In the G1 phase, enzymes required to duplicate DNA are synthesized. During the S phase, the enzymes made in the G1 phase create a copy of the chromosomes, resulting in two identical sets. In the G2 phase, protein synthesis is carried out, particularly of microtubules. Finally, in the M phase the two daughter cells separate by a process including nuclear division, cytoplasmic division, and formation of a new cell membrane.

Due to the complexity of cell division, there is a correspondingly complex system of cell signaling networks that regulate the progression through the G1, S, G2 and M phases. Normal signaling may come from, for example, receptor proteins, inflammatory factors, or pro- or anti-apoptotic signals. Dysfunctional signals can cause disease states and uncontrolled cell proliferation, and arise from, for example, genetic mutations, alterations, or overexpression, infection, exposure to environmental factors including toxins, system stress, autoimmune disorders, and inflammation. A range of disorders can result from dysfunctional signaling, including abnormal cellular proliferation such as cancer, tumors, autoimmune disorders, inflammatory disorders, graft-versus-host rejection, and fibrotic disorders.

A class of enzymes referred to as cyclin-dependent kinases (CDKs) are critical to regulating cell division. CDKs are generally involved in regulating transcription, mRNA processing, and differentiation of nerve cells. Each CDK binds to its own regulatory protein referred to as a cyclin. Without cyclin, the CDK has little or no kinase activity. CDKs phosphorylate their substrates on serine or threonine, and therefore are serine-threonine kinases. They are heterodimeric complexes composed of a catalytic kinase subunit and a regulatory cyclin subunit. CDK activity is controlled by association with their corresponding regulatory subunits (cyclins) and CDK inhibitor proteins (Cip & Kip proteins, INK4s), by their phosphorylation state, and by ubiquitin-mediated proteolytic degradation (see D. G. Johnson, C. L. Walker, Annu. Rev. Pharmacol. Toxicol 39 (1999) 295-312; D. O. Morgan, Annu. Rev. Cell Dev. Biol. 13 (1997) 261-291; C. J. Sherr, Science 274 (1996) 1672-1677; T. Shimamura et al., Bioorg. Med. Chem. Lett. 16 (2006) 3751-3754).

There are four CDKs that are significantly involved in cellular proliferation: CDK1, which predominantly regulates the transition from G2 to M phase, and CDK2, CDK4, and CDK6, which regulate the transition from G1 to S phase (Malumbres M, Barbacid M. Cell cycle, CDKs and cancer: a changing paradigm. Nat. Rev. Cancer 2009; 9(3):153-166). In early to mid G1 phase, when the cell is responsive to mitogenic stimuli, activation of CDK4-cyclin D and CDK6-cyclin D induces phosphorylation of the retinoblastoma protein (pRb).

Phosphorylation of pRb releases the transcription factor E2F, which enters the nucleus to activate transcription of other cyclins which promote further progression of the cell cycle (see J. A. Diehl, Cancer Biol. Ther. 1 (2002) 226-231; C. J. Sherr, Cell 73 (1993) 1059-1065).

The CDK enzymes interact with regulatory subunits known as cyclins for their activation. At least sixteen cyclins have been identified in mammals (Johnson D G, Walker C L. Cyclins and Cell Cycle Checkpoints. Annu. Rev. Pharmacol. Toxicol. (1999) 39:295-312). Among them cyclin B/CDK1, cyclin A/CDK2, cyclin E/CDK2, cyclin D/CDK4, and cyclin D/CDK6 play critical roles in regulating cell division, along with potentially others. Besides cell division regulation, cyclin/CDK pairs are also key to regulating processes like transcription, DNA repair, differentiation, and apoptosis (Morgan D O. Cyclin-dependent kinases: engines, clocks, and microprocessors. Annu. Rev. Cell. Dev. Biol. (1997) 13:261-291).

CDK2 overexpression is associated with atypical cell division regulation because of the role the cyclin E/CDK2 complex plays in G1/S transition, histone biosynthesis, and centrosome duplication. In general, CDK2 participates in the G1, S and G2 phases. The G1 transcription factor, E2F, is released when Rb is progressively phosphorylated by cyclin D/CDK4/6 and cyclin E/CDK2, promoting S phase entry. Additionally, activation of cyclin A/CDK2 in early S phase is partially responsible for the cascade that progresses to S phase completion (Asghar et al. The history and future of targeting cyclin-dependent kinases in cancer therapy, Nat. Rev. Drug. Discov. 2015; 14(2): 130-146).

Several CDK2 selective inhibitors in clinical trials (TG02, Tragara Pharmaceuticals; Milciclib, Tiziana Life Sciences; CYC065, Cyclacel Pharmaceuticals; Roniciclib, Bayer; Dinaciclib, Merck & Co.; AT7519, Astex Therapeutics Ltd).

The Scripps Research Institute filed three PCT Applications, WO2015/188120, WO2018/102433, and WO2019/139979 describing fluorosulfur(VI) compounds and uses thereof via reactions with phenols. This chemistry, known as SuFEx (Sulfur-Fluoride Exchange), has also been studied by the Sharpless lab at the Scripps Research Institute, which has published a number of papers on the topic (Dong et al. Angew. Chem. Int. Ed. Engl. 53(36), 9466-9470 (2014); Qin et al. Angew. Chem. Int. Ed. 55(45), 14155-14158 (2016); Gao et al. Angew. Chem. Int. Ed. 57(7), 1939-1943 (2018); Guo et al. Angew. Chem. Int. Ed. 57(10), 2605-2610 (2017); Gahtory et al. Chemistry 24(41), 10550-10556, (2018); Smedley et al. Angew. Chem. Int. Ed. 58(14), 4552-4556 (2019); Liu et al. Angew. Chem. Int. Ed. 58(24), 8029-8033 (2019); Dong et al. Angew. Chem. Int. Ed. 53(36), 9430-9448 (2014); Li et al. Angew. Chem. Int. Ed. 56(11), 2903-2908 (2017); Zheng et al. PNAS 116(38) 18808-18814 (2019); Wang et al. Angew. Chem. Int. Ed. 56(37), 11203-11208 (2017); Liu et al. J. Am. Chem. Soc. 140, 2919-2925 (2018); and Chen et al. J. Am. Chem. Soc. 138, 7353-7364 (2016)). Similar sulfonyl fluoride chemistries have been developed for the purpose of biorthogonal protein labelling (Narayanan et al. Chem. Sci. 6(5): 2650-2659 (2015) and Gu et al. J. Chem. Biol. 20(4), 541-548 (2013)) These strategies employ electrophilic sulfonyl compounds with a fluoride leaving group to react with a variety of nucleophiles.

Ku-Lung Hsu, et al., at University of Virginia have described sulfonyl-containing heteroaryl compound which have been named SuTEx compounds (Sulfur-Triazole Exchange)(Hahm et al. Nat. Chem. Bio. 16, 150-159 (2020); Brulet et al. J. Am. Chem. Soc. 142(18), 8270-8280 (2020); Borne et al. Development and biological applications of sulfur-triazole exchange (SuTEx) chemistry RSC Chem. Biol. (2021); and Huang et al. Chemoproteomic profiling of kinases in live cells using electrophilic sulfonyl triazole probes Chem. Sci. (2021)). See also WO 2020/214336 (Sulfur-heterocycle exchange chemistry) and WO 2021/016263 (Cysteine Binding Compositions and Methods of Use Thereof), filed by University of Virginia as assignee, and Hsu, et al. as inventors. Additional publications on the use of SuTEx molecules include Grams et al. Reactive chemistry for covalent probe and therapeutic development Trends in Pharmacological Sciences (2022) and Toroitich et al. Discovery off a cell-active SuTEx ligand of prostaglandin reductase 2 ChemBioChem (2021).

It is an object of the present invention to provide new compositions of matter and their methods of use and manufacture to treat diseases mediated by CDK2.

### SUMMARY OF THE INVENTION

Heteroaryl sulfonyl compounds and their uses and manufacture are provided that covalently modify CDK2 to treat a disease mediated by CDK2 in a host, typically a human. The heteroaryl sulfonyl compound is first typically selectively non-covalently bound to CDK2 by association of CDK2 with a CDK2 Recognition Moiety in the heteroaryl sulfonyl compound. In a typical second step, a reactive tyrosine residue on CDK2 attacks the sulfonyl moiety in the heteroaryl sulfonyl compound of the present invention to form a covalent bond between the tyrosine and the compound and force the elimination of a Leaving Group from the compound. In another aspect a reactive lysine residue on CDK2 attacks the heteroaryl sulfonyl compound of the present invention to form a covalent bond between the lysine and the compound and force the elimination of a Leaving Group from the compound.

The heteroaryl sulfonyl compounds of the present invention are uniquely designed for CDK2 specificity to maximize therapeutic effect and minimize off-target toxicity, by inclusion of specific CDK2 Recognition Moieties as described further herein that selectively bind CDK2 for further covalent linkage. In this way, the heteroaryl sulfonyl compound of the present invention exerts precise control over the targeted silencing, destruction, or inactivation of CDK2 while limiting unacceptable off-target effects.

CDK2 Recognition Moiety is a molecule that has a functional group linking it into the heteroaryl sulfonyl compound of the present invention, and is, for example a synthetic or naturally occurring small molecule that binds to CDK2 as an inhibitor or alternatively has no apparent biological effect on CDK2. In non-limiting embodiments, the CDK2 Recognition Moiety is a protein binding domain of a drug or pharmaceutically active compound which modulates CDK2 (or the full drug or pharmaceutically active compound). In alternative embodiments, the CDK2 Recognition Moiety may be a peptide, RNA or DNA oligonucleotide, or another biologic compound or fragment thereof, which can be suitably stabilized as necessary.

The heteroaryl sulfonyl compounds described herein can take advantage of the variable electrophilic properties of heteroaryl sulfonyl compounds to covalently modify CDK2, resulting in a decrease or termination of its biological activity.

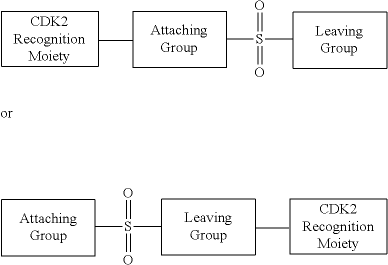

The covalent-binding heteroaryl sulfonyl compounds of the present invention include a CDK2 Recognition Moiety, a Leaving Group, and an Attaching Group. The heteroaryl sulfonyl compounds are oriented such that the Leaving Group is on one side of the S(O)₂ electrophile and the Attaching Group is on the other. The CDK2 Recognition Moiety is located either on the Leaving Group or the Attaching Group in a manner that allows it to associate with CDK2 as described herein.

In some embodiments, the Leaving Group is a monocyclic or bicyclic heteroaryl group bound to the sulfur atom through a S—N bond. For example, as used herein, R¹ and R⁴ are typically Leaving Groups. The Leaving Group is eliminated when the heteroaryl sulfonyl compound undergoes nucleophilic attack by a nucleophilic amino acid, for example a tyrosine or lysine, of CDK2. The Attaching Group, and the S(O)₂ group to which it is bound, remain bonded to CDK2 after covalent modification. For example, as used herein, R², R⁵, and R¹³ are Attaching Groups.

A non-limiting example of the covalent modification of CDK2 via a tyrosine that reacts with the heteroaryl sulfonyl compound of the present invention is provided below:

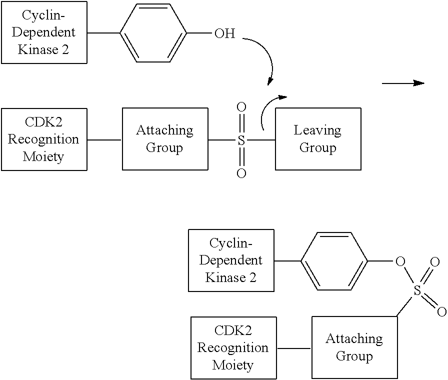

The CDK2 Recognition Moiety brings the activated heteroaryl sulfonyl compound of the present invention into close proximity with a reactive nucleophilic amino acid of CDK2 resulting in covalent modification of CDK2 and resultant amelioration or elimination of a disease or CDK2-mediated disorder.

The heteroaryl sulfonyl compound of the present invention is used to modulate CDK2's biological activity by covalently modifying the protein, for example, by covalently modifying a tyrosine in or near the active site, or alternatively, a lysine moiety.

In certain aspects, a heteroaryl sulfonyl compound of Formula I, Formula IL, Formula III, Formula IV, Formula V, Formula VI, or Formula VII, is provided:

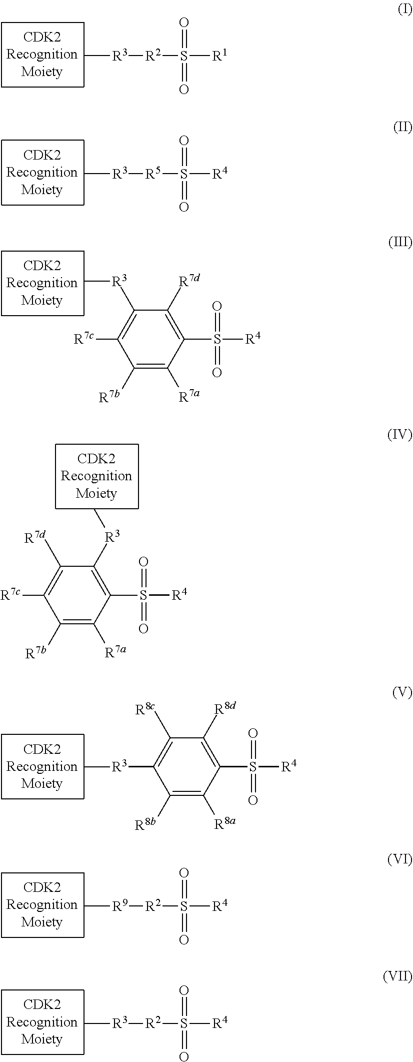

or a pharmaceutically acceptable salt, N-oxide, isotopic derivative, or prodrug thereof, optionally in a pharmaceutically acceptable carrier to form a pharmaceutical composition;
wherein:

R¹ is selected from:
a)

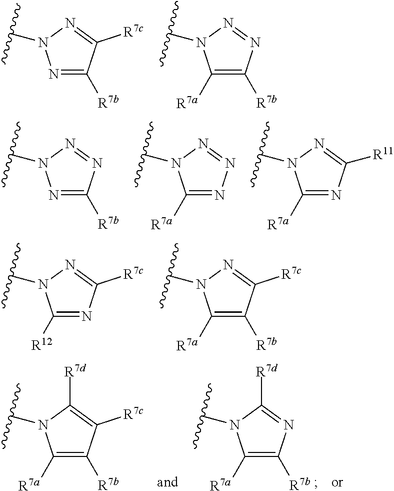

b) a bicyclic heteroaryl or tricyclic heteroaryl, optionally substituted as allowed by valence with 1, 2, 3, or 4 substituents selected from R⁷;

R² is independently selected at each instance from bond, alkyl, alkenyl, haloalkyl, cycloalkyl, aryl, heterocycle, —S—, —O—, —NR⁶—, —(CH₂)ₚ—C(O)—, —(CH₂)ₚ—C(O)—NR⁶—, —(CH₂CH₂O)ₚ—, —(OCH₂CH₂)ₚ—, —C(O)—, —NR⁶C(O)—, —NR⁶C(O)NR⁶—, —C(O)NR⁶—, —OC(O)NR⁶—, —NR⁶S(O)₂NR⁶—, —S(O)₂NR⁶—, aryl-C(O)—NR⁶—, heteroaryl-C(O)—NR⁶—, bicycle, tricycle, and heteroaryl, each of which is optionally substituted as allowed by valence with 1, 2, 3, or 4 substituents selected from R⁷;

in principal embodiments R² is selected from alkyl, alkenyl, haloalkyl, cycloalkyl, aryl, bicycle, tricycle, and heteroaryl, each of which is optionally substituted as allowed by valence with 1, 2, 3, or 4 substituents selected from R⁷;

R³ is a bivalent moiety independently selected at each instance from bond, alkyl, alkenyl, haloalkyl, cycloalkyl, aryl, heterocycle, —S—, —O—, —NR⁶—, —(CH₂)ₚ—C(O)—, —(CH₂)ₚ—C(O)—NR⁶—, —(CH₂CH₂O)ₚ—, —(OCH₂CH₂)ₚ—, —C(O)—, —NR⁶C(O)—, —NR⁶C(O)NR⁶—, —C(O)NR⁶—, —OC(O)NR⁶—, —NR⁶S(O)₂NR⁶—, —S(O)₂NR⁶—, aryl-C(O)—NR⁶—, heteroaryl-C(O)—NR⁶—, bicycle, tricycle, and heteroaryl, each of which except bond is optionally substituted as allowed by valence with 1, 2, 3, or 4 substituents selected from R⁷;

or R³ is a bivalent moiety independently selected at each instance from bond, alkyl, alkenyl, haloalkyl, cycloalkyl, aryl, heterocycle, —S—, —O—, —NR⁶—, —S-alkyl-, —O-alkyl-, —NR⁶-alkyl-, -alkyl-C(O)—, -alkyl-C(O)-alkyl-, -alkyl-C(O)—NR⁶-alkyl-, —C(O)—NR⁶-alkyl-, -alkyl-C(O)—NR⁶—, -alkyl-C(O)—O-alkyl-, —C(O)—O-alkyl-, -alkyl-C(O)—O—, —C(O)—, —NR⁶C(O)NR⁶—, —C(O)NR⁶—, —OC(O)NR⁶—, —NR⁶S(O)₂NR⁶—, —S(O)₂NR⁶—, bicycle, tricycle, and heteroaryl, each of which except bond is optionally substituted as allowed by valence with 1, 2, 3, or 4 substituents selected from R⁷;

and wherein R² and R³ are selected such that a suitably stable and suitably nontoxic compound for in vivo administration in a host is achieved, and in certain embodiments, in a way that avoids undesired repetition of atoms or moieties, such as in nonlimiting examples, S, O, or a combination thereof (i.e., that would otherwise form a disulfide or peroxide bond), as well known to skilled artisans;

each p is independently selected from 1, 2, 3, 4, 5, and 6;

R⁴ is a heteroaryl group, where the bond to the sulfur atom is through one of the nitrogen atoms present in the cycle, and each heteroaryl is optionally substituted as allowed by valence with 1, 2, 3, or 4 substituents selected from R⁷;

R⁵ is a bivalent moiety selected from alkyl, alkenyl, haloalkyl, cycloalkyl, naphthyl, heterocycle, —S—, —O—, —NR⁶—, —(CH₂)ₚ—C(O)—, —(CH₂)ₚ—C(O)—NR⁶—, —(CH₂CH₂O)ₚ—, —(OCH₂CH₂)ₚ—, —C(O)—, —NR⁶C(O)—, —NR⁶C(O)NR⁶—, —C(O)NR⁶—, —OC(O)NR⁶—, —NR⁶S(O)₂NR⁶—, —S(O)₂NR⁶—, heteroaryl-C(O)—NR⁶—, bicycle, tricycle, and heteroaryl, each of which is optionally substituted as allowed by valence with 1, 2, 3, or 4 substituents selected from R⁷;

in principal embodiments R⁵ is selected from alkyl, alkenyl, haloalkyl, cycloalkyl, naphthyl, heterocycle, bicycle, tricycle, and heteroaryl, each of which is optionally substituted as allowed by valence with 1, 2, 3, or 4 substituents selected from R⁷;

and wherein R³ and R⁵ are selected such that a suitably stable and suitably nontoxic compound for in vivo administration in a host is achieved, and in certain embodiments, in a way that avoids undesired repetition of atoms or moieties, such as in nonlimiting examples, S, O, or a combination thereof (i.e., that would otherwise form a disulfide or peroxide bond), as well known to skilled artisans;

R⁶ is independently selected at each instance from hydrogen, alkyl, haloalkyl, cycloalkyl, aryl, heterocycle, and heteroaryl; each of which except hydrogen is optionally substituted as allowed by valence with 1, 2, 3, or 4 substituents selected from R¹⁸;

R⁷, R^{7a}, R^{7b}, R^{7c}, and R^{7d} are independently selected at each instance from hydrogen, halogen, alkyl, haloalkyl, alkenyl, cycloalkyl, heterocycle, aryl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, —S(O)R⁶, —S(O)₂R⁶, —S(O)OR⁶, —S(O)₂OR⁶, —S(O)N(R⁶)₂, S(O)₂N(R⁶)₂, ═O, and —SR⁶, wherein each alkyl, haloalkyl, alkenyl, cycloalkyl, heterocycle, aryl, and heteroaryl is optionally substituted as allowed by valence with 1, 2, 3, or 4 substituents selected from R¹⁷;

R¹⁷ is independently selected in each instance from hydrogen, halogen, alkyl, haloalkyl, alkenyl, cycloalkyl, heterocycle, aryl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, —S(O)R⁶, —S(O)₂R⁶, —S(O)OR⁶, —S(O)₂OR⁶, —S(O)N(R⁶)₂, S(O)₂N(R⁶)₂, ═O, and —SR;

R¹⁸ is independently selected in each instance from hydrogen, halogen, alkyl, haloalkyl, alkenyl, cycloalkyl, heterocycle, aryl, heteroaryl, cyano, nitro, —C(O)R¹⁹, —OC(O)R¹⁹, —NR¹⁹C(O)R¹⁹, —C(O)OR¹⁹, —OC(O)OR¹⁹, —NR¹⁹C(O)OR¹⁹, —C(O)N(R¹⁹)₂, —OC(O)N(R¹⁹)₂, —NR¹⁹C(O)N(R¹⁹)₂, —OR¹⁹, —N(R¹⁹)₂, —S(O)R¹⁹, —S(O)₂R¹⁹, —S(O)OR¹⁹, —S(O)₂OR¹⁹, —S(O)N(R¹⁹)₂, S(O)₂N(R¹⁹)₂, ═O, and —SR¹⁹;

R¹⁹ is independently selected at each instance from hydrogen, alkyl, haloalkyl, cycloalkyl, aryl, heterocycle, and heteroaryl;

R^{8a}, R^{8b}, R^{8c}, and R^{8d} are independently selected at each instance from R⁷ and R¹² wherein at least one of R^{8a}, R^{8b}, R^{8c}, and R^{8d} is R¹²;

R⁹ is a bivalent moiety selected from alkyl, alkenyl, haloalkyl, cycloalkyl, heterocycle, —NR⁶C(O)—, —NR⁶C(O)NR⁶—, —C(O)NR⁶—, —OC(O)NR⁶—, —NR⁶S(O)₂NR⁶—, —S(O)₂NR⁶—, bicycle, tricycle, and heteroaryl, each of which is optionally substituted as allowed by valence with 1, 2, 3, or 4 substituents selected from R⁷;

or R⁹ is a bivalent moiety selected from alkyl, alkenyl, haloalkyl, cycloalkyl, heterocycle, -alkyl-C(O)—NR⁶-alkyl-, —C(O)—NR⁶-alkyl-, -alkyl-C(O)—NR⁶—, -alkyl-C(O)—O-alkyl-, —C(O)—O-alkyl-, -alkyl-C(O)—O—, —NR⁶C(O)—, —NR⁶C(O)NR⁶—, —C(O)NR⁶—, —OC(O)NR⁶—, —NR⁶S(O)₂NR⁶—, —S(O)₂NR⁶—, bicycle, tricycle, and heteroaryl, each of which is optionally substituted as allowed by valence with 1, 2, 3, or 4 substituents selected from R⁷;

R¹¹ is selected from hydrogen, halogen, alkyl, haloalkyl, alkenyl, cycloalkyl, heterocycle, naphthyl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, and —SR⁶, wherein each alkyl, haloalkyl, alkenyl, cycloalkyl, heterocycle, aryl, and heteroaryl is optionally substituted as allowed by valence with 1, 2, 3, or 4 substituents selected from R⁷; and

R¹² is independently selected from halogen, alkyl, haloalkyl, alkenyl, cycloalkyl, heterocycle, aryl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, —S(O)R⁶, —S(O)₂R⁶, —S(O)OR⁶, —S(O)₂OR⁶, —S(O)N(R⁶)₂, S(O)₂N(R⁶)₂, ═O, and —SR⁶, wherein each alkyl, haloalkyl, alkenyl, cycloalkyl, heterocycle, aryl, and heteroaryl is optionally substituted as allowed by valence with 1, 2, 3, or 4 substituents selected from R¹⁷; and

CDK2 Recognition Moiety is a molecule which can bind to or otherwise anchors to CDK2.

In certain embodiments CDK2 has a reactive tyrosine which covalently binds to a selected heteroaryl sulfonyl compound of the present invention. In certain embodiments the reactive tyrosine is in the active site. In certain embodiments the reactive tyrosine is not in the active site.

In certain embodiments a heteroaryl sulfonyl compound of the present invention covalently modifies Tyr15 of CDK2.

In certain embodiments CDK2 has a reactive lysine which covalently binds to the heteroaryl sulfonyl compounds of the present invention. In certain embodiments the reactive lysine is in the active site. In certain embodiments the reactive lysine is not in the active site.

In certain embodiments CDK2 has a reactive cysteine which covalently binds to the heteroaryl sulfonyl compounds of the present invention. In certain embodiments the reactive cysteine is in the active site. In certain embodiments the reactive cysteine is not in the active site.

Assays and/or spectroscopic techniques to confirm covalent binding are described in the paper by Brulet et. al. titled “Liganding Functional Tyrosine Sites on Proteins Using Sulfur-Triazole Exchange Chemistry” JACS 2020, 142, 8270-8280 or the paper by Hahm et. al. titled “Global targeting of functional tyrosines using sulfur triazole exchange chemistry” Nature Chem. Biol. 2020, 16(2), 150-159.

In principle embodiments, the CDK2 Recognition Moiety is neither a fluorophore, nor a detectable labeling group, nor a moiety comprising an alkyne. In certain embodiments the CDK2 Recognition Moiety is selective for CDK2 and is not a non-selective protein binder, for example a promiscuous binder of a range of enzymes or of kinases, such as CDKs generally. As one nonlimiting example, the CDK2 Recognition Moiety is not a pan-kinase inhibitor such as:

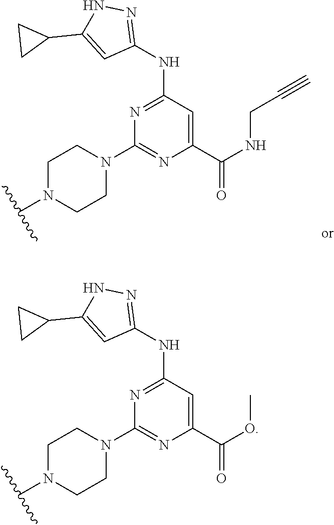 [SMILES: *N1CCN(c2nc(Nc3cc(C4CC4)[nH]n3)cc(C(=O)NCC#C)n2)CC1 ; *N1CCN(c2nc(Nc3cc(C4CC4)[nH]n3)cc(C(=O)OC)n2)CC1]

In principle embodiments, the heteroaryl sulfonyl compound of the present invention is also not a chemical probe used to perturb the function of a variety of proteins in a biological sample, but instead a focused CDK2 binder.

In certain embodiments the heteroaryl sulfonyl compound of the present invention primarily covalently modifies a specific tyrosine or lysine in CDK2. In other embodiments, a selected heteroaryl sulfonyl compound of the present invention reacts with two or more different tyrosines and/or lysines in CDK2. In certain embodiments the heteroaryl sulfonyl compound of the present invention is more than about 5-, 10-, 15-, 20-, 25-, 50-, 75-, or 100-fold more selective for one specific amino acid, for example a specific tyrosine, than other amino acids of CDK2.

In certain embodiments one or more nucleophilic amino acids other than tyrosine or lysine is covalently modified by a heteroaryl sulfonyl compound of the present invention. For example, the amino acid that is covalently modified is cysteine, arginine, histidine, serine, threonine, or tryptophan.

In another aspect a heteroaryl sulfonyl compound of Formula VIII is provided:

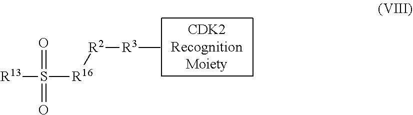

or a pharmaceutically acceptable salt, N-oxide, isotopic derivative, or prodrug thereof, optionally in a pharmaceutically acceptable carrier to form a composition;
wherein:

R¹³ is selected from hydrogen, alkyl, haloalkyl, cycloalkyl, heterocycle, heteroaryl, aryl, —OR⁶, —N(R⁶)₂, —C(O)R⁶, —NR⁶C(O)R⁶, —C(O)N(R⁶)₂, and —NR⁶C(O)N(R⁶)₂, each of which except hydrogen is optionally substituted as allowed by valence with 1, 2, 3, or 4 substituents selected from R⁷;

R¹⁶ is a heteroaryl group, where the bond to the sulfur atom is through one of the nitrogen atoms present in the cycle, and R¹⁶ is optionally substituted as allowed by valence with 1, 2, 3, or 4 substituents independently selected from R⁷; and all other variables are as defined herein.

In a typical embodiment, R² is selected in each instance from bond, alkyl, alkenyl, haloalkyl, cycloalkyl, aryl, heterocycle, —(CH₂)ₚ—C(O)—, —(OCH₂CH₂)ₚ—, —C(O)—, —NR⁶C(O)—, and heteroaryl, each of which is optionally substituted as allowed by valence with 1, 2, 3, or 4 substituents selected from R⁷, wherein if R² is bond, R³ is R^{3*}; wherein

R^{3*} is selected in each instance from bond, alkyl, alkenyl, haloalkyl, cycloalkyl, aryl, heterocycle, —(CH₂)ₚ—C(O)—, —(OCH₂CH₂)ₚ—, —C(O)—, —NR⁶C(O)—, bicycle, and heteroaryl, each of which is optionally substituted as allowed by valence with 1, 2, 3, or 4 substituents selected from R⁷.

In a typical embodiment, R⁵ is selected in each instance from bond, alkyl, alkenyl, haloalkyl, cycloalkyl, aryl, heterocycle, —(CH₂)ₚ—C(O)—, —(OCH₂CH₂)ₚ—, —C(O)—, —NR⁶C(O)—, and heteroaryl, each of which is optionally substituted as allowed by valence with 1, 2, 3, or 4 substituents selected from R⁷.

In other embodiments R⁵ is selected from alkyl, alkenyl, haloalkyl, cycloalkyl, naphthyl, heterocycle, —(OCH₂CH₂)ₚ—, —C(O)—, —NR⁶C(O)—, bicycle, and heteroaryl, each of which is optionally substituted as allowed by valence with 1, 2, 3, or 4 substituents selected from R⁷.

In one aspect a heteroaryl sulfonyl compound of Formula IX is provided:

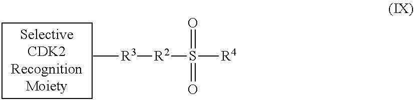

or a pharmaceutically acceptable salt, N-oxide, isotopic derivative, or prodrug thereof, optionally in a pharmaceutically acceptable carrier to form a composition;
wherein Selective CDK2 Recognition Moiety is a CDK2 Recognition Moiety as defined herein wherein at least one of the following is satisfied:
i. there are fewer than 80, 70, 60, 50, 40, 30, 20, 15, 10, or 5 endogenous protein kinases to which the Selective CDK2 Recognition Moiety binds with an KD50 of 2 μM or less in an in vitro assay, for example the full KinaseProfiler kinase screening assay or scanEDGE kinase screening assay by Eurofins Discovery; or
ii. the Selective CDK2 Recognition Moiety has an KD50 greater than 10 μM against aurora B kinase, c-Src kinase domain, human serine/threonine-protein kinase (MST4), activin receptor type-IIA (ACVR2A), human calcium calmodulin dependent protein kinase II delta isoform 1 (CAMKD), and/or human ste20-like kinase;
   and wherein all other variables are as defined herein.

In certain embodiments of the invention, the CDK2 Recognition Moiety is a small organic molecule (i.e., a non-biologic) that adequately binds to CDK2 in a manner that it is covalently modified. In other embodiments of the invention, the CDK2 Recognition Moiety is a peptide or oligonucleotide that adequately binds to the protein in such a manner that it is covalently modified.

In certain embodiments the CDK2 Recognition Moiety is a residue of a pharmaceutically active compound that binds to CDK2 (for example but not limited to a compound of the sort that would be reviewed as a drug by CDER of the FDA, or an approved or clinical stage drug) or a peptide, protein or biologic or a binding fragment thereof that adequately binds to the protein in such a manner that it is covalently modified. A plethora of illustrative nonlimiting examples of CDK2 Recognition Moieties in the heteroaryl sulfonyl compound of the present invention are provided in the detailed description and additional CDK2 Recognition Moieties are readily apparent.

In certain embodiments the CDK2 Recognition Moiety is

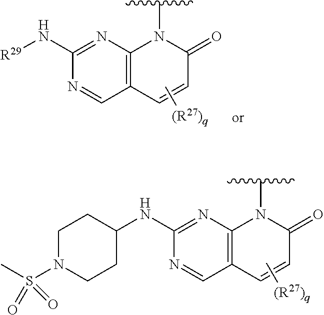

wherein

R²⁹ is independently selected at each instance from hydrogen, alkyl, haloalkyl, cycloalkyl, aryl, heterocycle, and heteroaryl each of which is optionally substituted with 1 or 2 substituents independently selected from R¹⁷ for example SO₂Me;

q is 0, 1, 2, or 3;

and wherein all other variables are as defined herein.

In certain embodiments q is 0. In certain embodiments q is 1.

In certain embodiments the CDK2 Recognition Moiety is

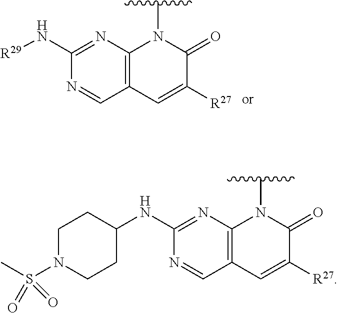

The present invention focuses on the covalent modification of CDK2 to treat diseases, for example, abnormal cellular proliferation such as tumors and cancer. In certain embodiments, a method of treating a disorder mediated by a CDK2 is provided comprising administering an effective amount of a heteroaryl sulfonyl compound of Formula I, Formula II, Formula III, Formula IV, Formula V, Formula VI, Formula VII, Formula VIII, or Formula IX, to a patient in need thereof, for example a human, or a pharmaceutically acceptable salt thereof optionally in a pharmaceutically acceptable carrier. For example, in one embodiment, a heteroaryl sulfonyl compound of Formula I, Formula IL, Formula III, Formula IV, Formula V, Formula VI, Formula VII, Formula VIII, or Formula IX, is administered to a human to treat a cancer or tumor where the heteroaryl sulfonyl compound has a CDK2 Recognition Moiety and the tumor or cancer is mediated by CDK2.

In certain embodiments, the heteroaryl sulfonyl compound described herein does not have to be administered in as high of a dose or as frequently as the classic CDK2 inhibitor corresponding to the CDK2 Recognition Moiety incorporated into the heteroaryl sulfonyl compound alone for treatment of a disorder. In certain embodiments, a heteroaryl sulfonyl compound of the present invention has fewer or less severe side-effects in the treatment of a disorder mediated by CDK2, than the classic CDK2 inhibitor corresponding to the CDK2 Recognition Moiety incorporated into the heteroaryl sulfonyl compound alone. In certain embodiments, the heteroaryl sulfonyl compound of the present invention is more efficacious in the treatment of a disorder mediated by CDK2 than the classic CDK2 inhibitor corresponding to the CDK2 Recognition Moiety incorporated into the heteroaryl sulfonyl compound alone.

In certain embodiments, a heteroaryl sulfonyl compound described herein is useful to treat a disorder, for example abnormal cellular proliferation, such as a tumor or cancer, wherein CDK2 is mutated, altered, or overexpressed. In other embodiments a heteroaryl sulfonyl compound described herein is useful to treat a disorder for example abnormal cellular proliferation, such as a tumor or cancer, wherein CDK2 is not mutated. In certain embodiments a heteroaryl sulfonyl compound described herein is at least about 2-, 3-, 4-, 5-, 10-, 50-, 100-, 200-, 300-, 400-, 500-, or 1,000-fold more selective for a mutated CDK2 than the wild-type CDK2.

In certain embodiments, a heteroaryl sulfonyl compound of the present invention is useful as a therapeutic agent, when administered in an effective amount to a patient, for the treatment of a medical disorder that can be treated with the CDK2 Recognition Moiety.

The heteroaryl sulfonyl compounds of the present invention can be administered in any manner that allows the heteroaryl sulfonyl compound to covalently modify CDK2. As such, examples of methods to deliver the heteroaryl sulfonyl compounds of the present invention include, but are not limited to, systemic, parenteral, topical, oral, intravenous, buccal, sublingual, subcutaneous, or transnasal administration.

In certain embodiments, the heteroaryl sulfonyl compound of the present invention has at least one desired isotopic substitution of an atom, at an amount above the natural abundance of the isotope, i.e., enriched.

In one embodiment, the heteroaryl sulfonyl compound of the present invention includes a deuterium or multiple deuterium atoms. Deuterium is not considered or used herein as a detectable labeling group.

Another aspect of the present invention provides a heteroaryl sulfonyl compound as described herein, or an enantiomer, diastereomer, or stereoisomer thereof, or pharmaceutically acceptable salt, hydrate, or solvate thereof, or a pharmaceutical composition, for use in the manufacture of a medicament for treating or preventing a disease in which CDK2 plays a role.

In one embodiment, the heteroaryl sulfonyl compound of the present invention is not fluorescent, including but not limited to not a fluorophore.

In other aspects a heteroaryl sulfonyl compound of Formula I′, Formula II′, Formula III′, Formula IV′, Formula V′, Formula VI′, Formula VII′, Formula VIII′, or Formula IX′ is provided:

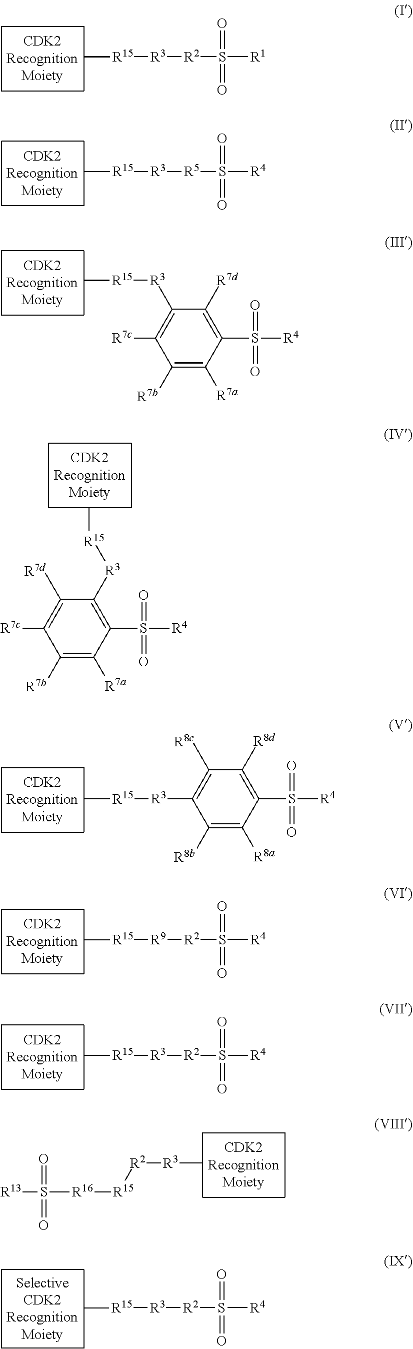

or a pharmaceutically acceptable salt, N-oxide, isotopic derivative, or prodrug thereof, optionally in a pharmaceutically acceptable carrier to form a pharmaceutical composition;

wherein R¹⁵ is a bivalent moiety selected from the group consisting of alkyl, alkenyl, haloalkyl, cycloalkyl, aryl, heterocycle, —S—, —O—, —NR⁶—, —S-alkyl-, —O-alkyl-, —NR⁶-alkyl-, -alkyl-C(O)—, -alkyl-C(O)-alkyl-, -alkyl-C(O)—NR⁶-alkyl-, —C(O)—NR⁶-alkyl-, -alkyl-C(O)—NR⁶—, -alkyl-C(O)—O-alkyl-, —C(O)—O-alkyl-, -alkyl-C(O)—O—, —C(O)—, —NR⁶C(O)NR⁶—, —C(O)NR⁶—, —OC(O)NR⁶—, —NR⁶S(O)₂NR⁶—, —S(O)₂NR⁶—, bicycle, tricycle, and heteroaryl, each of which except bond is optionally substituted as allowed by valence with 1, 2, 3, or 4 substituents independently selected from R⁷; and wherein all other variables are as defined herein.

Additional features and advantages of the present application will be apparent from the following detailed description.

The present invention thus includes at least the following features:
(a) A heteroaryl sulfonyl compound of Formula I, Formula IL, Formula III, Formula IV, Formula V, Formula VI, Formula VII, Formula VIII, or Formula IX, as described herein, or a pharmaceutically acceptable salt or isotopic derivative (including a deuterated derivative) thereof;
(b) A method for treating a disorder mediated by CDK2, comprising administering an effective amount of a heteroaryl sulfonyl compound of Formula I, Formula II, Formula III, Formula IV, Formula V, Formula VI, Formula VII, Formula VIII, or Formula IX, or pharmaceutically acceptable salt thereof, as described herein, to a patient in need thereof;
(c) A heteroaryl sulfonyl compound of Formula I, Formula IL, Formula III, Formula IV, Formula V, Formula VI, Formula VII, Formula VIII, or Formula IX, or a pharmaceutically acceptable salt thereof for use in the treatment of a disorder that is mediated by CDK2;
(d) Use of a heteroaryl sulfonyl compound of Formula I, Formula IL, Formula III, Formula IV, Formula V, Formula VI, Formula VII, Formula VIII, or Formula IX, or a pharmaceutically acceptable salt thereof, in an effective amount in the treatment of a patient in need thereof, typically a human, with disorder mediated by CDK2;
(e) Use of a heteroaryl sulfonyl compound of Formula I, Formula IL, Formula III, Formula IV, Formula V, Formula VI, Formula VII, Formula VIII, or Formula IX, or a pharmaceutically acceptable salt thereof in the manufacture of a medicament for the treatment of a disorder mediated by CDK2;
(f) A pharmaceutical composition comprising an effective host-treating amount to a host in need thereof of a heteroaryl sulfonyl compound of Formula I, Formula II, Formula III, Formula IV, Formula V, Formula VI, Formula VII, Formula VIII, or Formula IX, or a pharmaceutically acceptable salt thereof, and a pharmaceutically acceptable carrier or diluent;
(g) A heteroaryl sulfonyl compound of Formula I, Formula II, Formula III, Formula IV, Formula V, Formula VI, Formula VII, Formula VIII, or Formula IX, as described herein as a mixture of enantiomers or diastereomers (as relevant), including as a racemate;
(h) A heteroaryl sulfonyl compound of Formula I, Formula II, Formula III, Formula IV, Formula V, Formula VI, Formula VII, Formula VIII, or Formula IX, as described herein in enantiomerically or diastereomerically (as relevant) enriched form, including an isolated enantiomer or diastereomer (i.e., greater than 85, 90, 95, 97, or 99% pure); and
(i) A process for the preparation of therapeutic products that contain an effective amount of a heteroaryl sulfonyl compound of Formula I, Formula II, Formula III, Formula IV, Formula V, Formula VI, Formula VII, Formula VIII, or Formula IX, or a pharmaceutically acceptable salt thereof, as described herein.
(j) A heteroaryl sulfonyl compound of Formula I′, Formula II′, Formula III′, Formula IV′, Formula V′, Formula VI′, Formula VII′, Formula VIII′, or Formula IX′, as described herein, or a pharmaceutically acceptable salt or isotopic derivative (including a deuterated derivative) thereof,
(k) A method for treating a disorder mediated by CDK2, comprising administering an effective amount of a heteroaryl sulfonyl compound of Formula I′, Formula II′, Formula III′, Formula IV′, Formula V′, Formula VI′, Formula VII′, Formula VIII′, or Formula IX′, or pharmaceutically acceptable salt thereof, as described herein, to a patient in need thereof;
(l) A heteroaryl sulfonyl compound of Formula I′, Formula II′, Formula III′, Formula IV′, Formula V′, Formula VI′, Formula VII′, Formula VIII′, or Formula IX′, or a pharmaceutically acceptable salt thereof for use in the treatment of a disorder that is mediated by CDK2;
(m) Use of a heteroaryl sulfonyl compound of Formula I′, Formula II′, Formula III′, Formula IV′, Formula V′, Formula VI′, Formula VII′, Formula VIII′, or Formula IX′, or a pharmaceutically acceptable salt thereof, in an effective amount in the treatment of a patient in need thereof, typically a human, with disorder mediated by CDK2;
(n) Use of a heteroaryl sulfonyl compound of Formula I′, Formula II′, Formula III′, Formula IV′, Formula V′, Formula VI′, Formula VII′, Formula VIII′, or Formula IX′, or a pharmaceutically acceptable salt thereof in the manufacture of a medicament for the treatment of a disorder mediated by CDK2;
(o) A pharmaceutical composition comprising an effective host-treating amount to a host in need thereof of a heteroaryl sulfonyl compound of Formula I′, Formula II′, Formula III′, Formula IV′, Formula V′, Formula VI′, Formula VII′, Formula VIII′, or Formula IX′, or a pharmaceutically acceptable salt thereof, and a pharmaceutically acceptable carrier or diluent;
(p) A heteroaryl sulfonyl compound of Formula I′, Formula II′, Formula III′, Formula IV′, Formula V′, Formula VI′, Formula VII′, Formula VIII′, or Formula IX′, as described herein as a mixture of enantiomers or diastereomers (as relevant), including as a racemate;
(q) A heteroaryl sulfonyl compound of Formula I′, Formula II′, Formula III′, Formula IV′, Formula V′, Formula VI′, Formula VII′, Formula VIII′, or Formula IX′, as described herein in enantiomerically or diastereomerically (as relevant) enriched form, including an isolated enantiomer or diastereomer (i.e., greater than 85, 90, 95, 97, or 99% pure); and
(r) A process for the preparation of therapeutic products that contain an effective amount of a heteroaryl sulfonyl compound of Formula I′, Formula II′, Formula III′, Formula IV′, Formula V′, Formula VI′, Formula VII′, Formula VIII′, or Formula IX′, or a pharmaceutically acceptable salt thereof, as described herein.

### BRIEF DESCRIPTION OF THE FIGURES

FIGS. 1A, 1B, 1C, and 1D presents non-limiting examples of ligands that bind to cyclin dependent kinase 2 (CDK2), including the compounds MTW, AJR, 1RO, 4SP, FB8, FC8, 0S0, B49, TIY, 3TI, STU, JWS, 2AN, SU9, U55, INR, CK2, CK8, RRC, F9Z, 23D, 8QT, 174, U32, U73, LS5, LS4, LS3, LS2, LS1, LQ5, 55S, 72L, C62, C96, CT8 and 5BN. For additional non-limiting examples and related ligands, see ligands identified by Kontopidis et al., “Differential Binding of Inhibitors to Active and Inactive Cdk2 Provides Insights for Drug Design”, Chem Biol., 2006, 13: 201; Talapati et al., “Structure of cyclin-dependent kinase 2 (CDK2) in complex with the specific and potent inhibitor CVT-313”, Acta Crystallogr F Struct Biol Commun., 2020, 76: 350-356; Echalier et al., “An integrated chemical biology approach provides insight into Cdk2 functional redundancy and inhibitor sensitivity”, Chem Biol., 2012, 19: 1028-1040; Wood et al., “Differences in the Conformational Energy Landscape of CDK1 and CDK2 Suggest a Mechanism for Achieving Selective CDK Inhibition”, Cell Chem Biol., 2019, 26: 121-130.e5; Martin et al., “A Novel Approach to the Discovery of Small-Molecule Ligands of CDK2”, Chembiochem., 2012, 13: 2128-2136; Betzi et al., “Discovery of a Potential Allosteric Ligand Binding Site in CDK2”, ACS Chem Biol., 2011, 6: 492-501; Clare et al., “The cyclin-dependent kinases cdk2 and cdk5 act by a random, anticooperative kinetic mechanism”, J Biol Chem., 2001, 276: 48292-48299; Davies et al., “Inhibitor Binding to Active and Inactive Cdk2: The Crystal Structure of Cdk2-Cyclin A/Indirubin-5-Sulphonate”, Structure, 2001, 9: 389; De Azevedo et al., “Inhibition of cyclin-dependent kinases by purine analogues: crystal structure of human cdk2 complexed with roscovitine”, Eur J Biochem., 1997, 243: 518-526; Whittaker et al., “Molecular profiling and combinatorial activity of CCT068127: a potent CDK2 and CDK9 inhibitor”, Mol Oncol., 2018, 12: 287-304; Hazel et al., “Inhibitor Selectivity for Cyclin-Dependent Kinase 7: A Structural, Thermodynamic, and Modelling Study”, ChemMedChem., 2017, 12: 372-380; Vulpetti et al., “Structure-Based Drug Design to the Discovery of New 2-Aminothiazole Cdk2 Inhibitors”, J Mol Graph Model., 2006, 24: 341; Bramson et al., “Oxindole-based inhibitors of cyclin-dependent kinase 2 (CDK2): design, synthesis, enzymatic activities, and X-ray crystallographic analysis”, J Med Chem., 2001, 44: 4339-4358; Alexander et al., “Type II Inhibitors Targeting Cdk2”, ACS Chem Biol., 2015, 10: 2116; Anscombe, et al., “Identification and Characterization of an Irreversible Inhibitor of CDK2”, Chem Biol., 2015, 22: 1159-1164; Coxon et al., “Cyclin-Dependent Kinase (CDK) Inhibitors: Structure-Activity Relationships and Insights into the CDK-2 Selectivity of 6-Substituted 2-Arylaminopurines”, J Med Chem., 2017, 60: 1746-1767; Richardson et al., “Discovery of a Potent Cdk2 Inhibitor with a Novel Binding Mode, Using Virtual Screening and Initial, Structure-Guided Lead Scoping”, Bioorg Med Chem Lett., 2007, 17: 3880; and Liu et al., “3,5,6-Trisubstituted Naphthostyrils as CDK2 Inhibitors”, Bioorg Med Chem., 2003, 13: 2465-2468.

FIG. 2 is a non-limiting example of a Formula described herein.

### DETAILED DESCRIPTION OF THE INVENTION

Heteroaryl sulfonyl compounds and their use and manufacture are provided that covalently modify CDK2 to treat a disease that is mediated by CDK2 in a host, typically a human. In one aspect of the invention a heteroaryl sulfonyl compound described herein reacts with a tyrosine residue on CDK2 to form a covalent bond. In another aspect a heteroaryl sulfonyl compound described herein reacts with a lysine residue on CDK2 to form a covalent bond. The invention provides a heteroaryl sulfonyl compound that includes a CDK2 Recognition Moiety that provides specificity to the heteroaryl sulfonyl compound, and an electrophilic sulfonyl (SO₂) that reacts with the target tyrosine or lysine to create a covalent bond between CDK2 and the presently described inhibitor.

The heteroaryl sulfonyl compound as described herein in principle embodiments has a stable shelf life for at least 2 months, 3 months, 6 months or 1 year or more neat or as part of a pharmaceutically acceptable dosage form, and itself is pharmaceutically acceptable.

In one aspect a heteroaryl sulfonyl compound of Formula I, Formula II, Formula III, Formula IV, Formula V, Formula VI, Formula VII, Formula VIII, or Formula IX, or a pharmaceutically acceptable salt thereof, is provided:

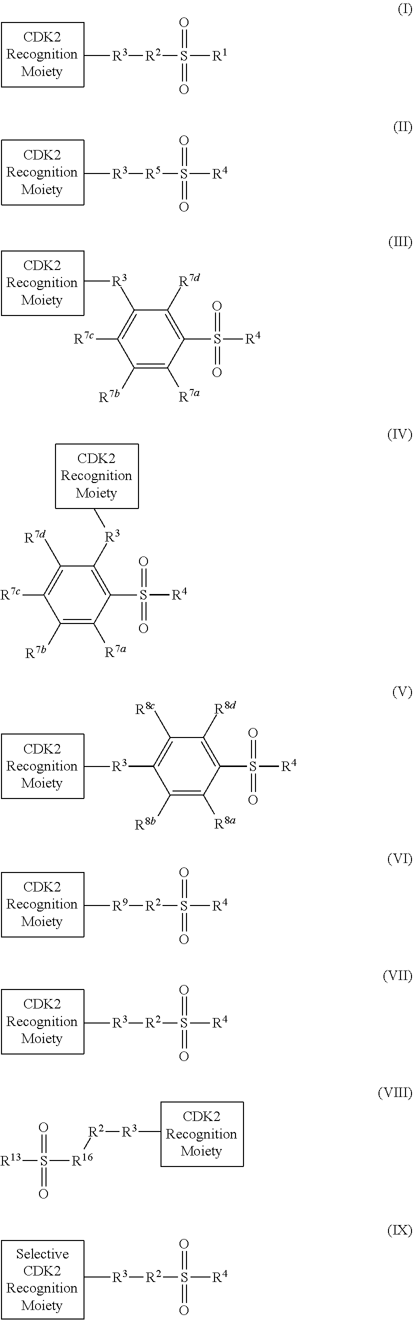

wherein the variables are as defined herein.

### Embodiments of Formula I

In certain embodiments the heteroaryl sulfonyl compound of Formula I is selected from:

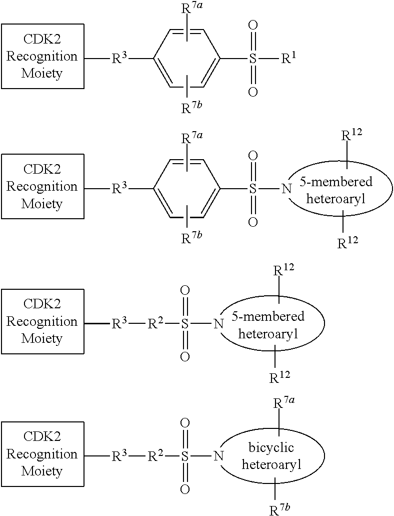

or a pharmaceutically acceptable salt thereof.

### Embodiments of Formula II

In certain embodiments the heteroaryl sulfonyl compound of Formula II is selected from:

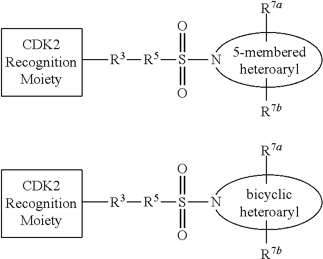

or a pharmaceutically acceptable salt thereof.

### Embodiments of Formula III

In certain embodiments the heteroaryl sulfonyl compound of Formula III is selected from:

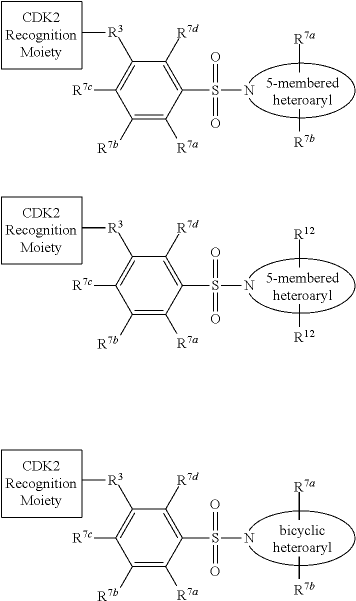

or a pharmaceutically acceptable salt thereof.

### Embodiments of Formula IV

In certain embodiments the heteroaryl sulfonyl compound of Formula IV is selected from:

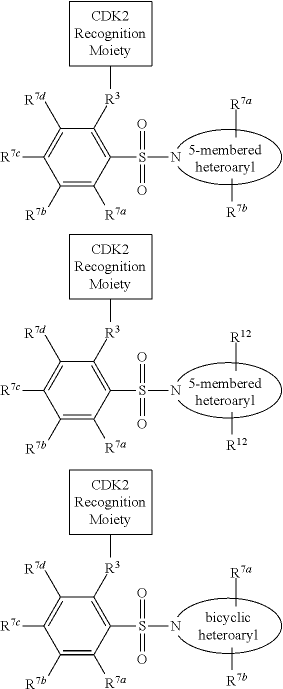

or a pharmaceutically acceptable salt thereof.

### Embodiments of Formula V

In certain embodiments the heteroaryl sulfonyl compound of Formula V is selected from:

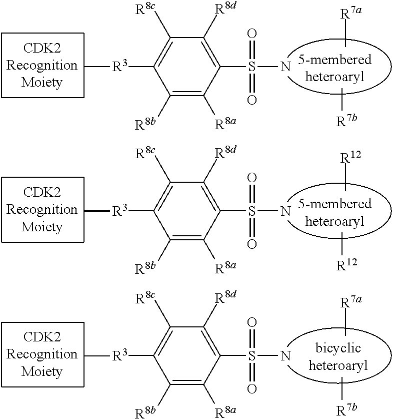

or a pharmaceutically acceptable salt thereof.

### Embodiments of Formula VI

In certain embodiments the heteroaryl sulfonyl compound of Formula VI is selected from:

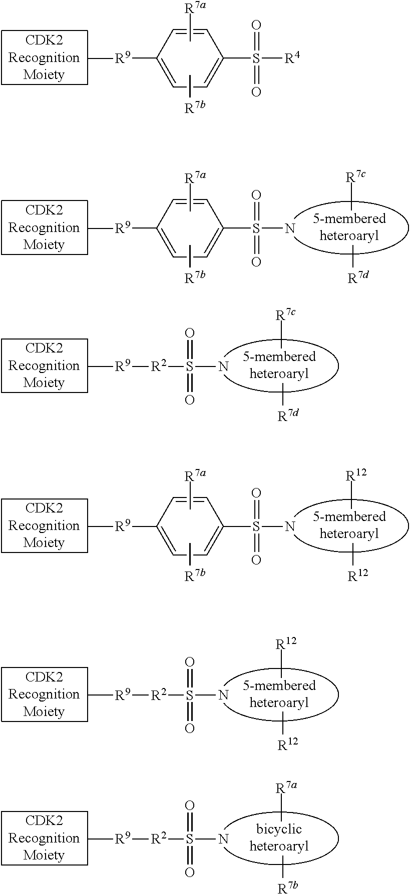

or a pharmaceutically acceptable salt thereof.

### Embodiments of Formula VII

In certain embodiments the heteroaryl sulfonyl compound of Formula VII is selected from:

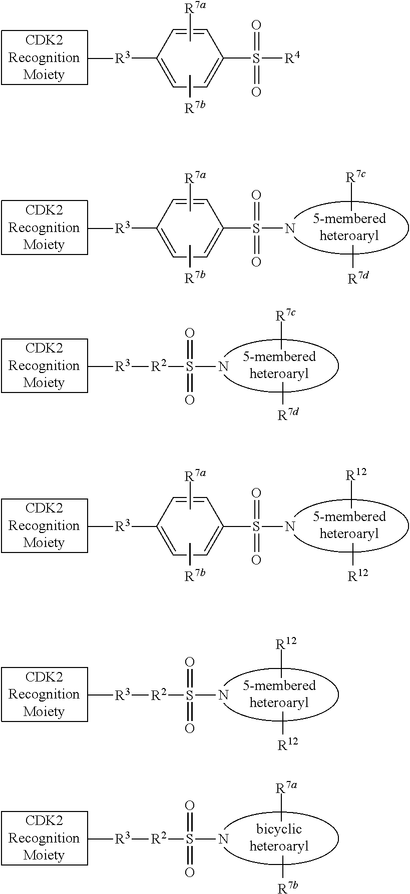

or a pharmaceutically acceptable salt thereof.

### Embodiments of Formula VIII

In certain embodiments the heteroaryl sulfonyl compound of Formula VIII is selected from:

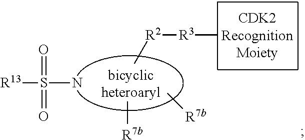

or a pharmaceutically acceptable salt thereof.

In certain embodiments the heteroaryl sulfonyl compound of Formula VIII is selected from:

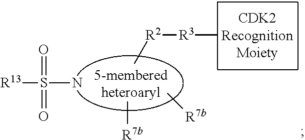

or a pharmaceutically acceptable salt thereof.

### Embodiments of Formula IX

In certain embodiments the heteroaryl sulfonyl compound of Formula IX is selected from:

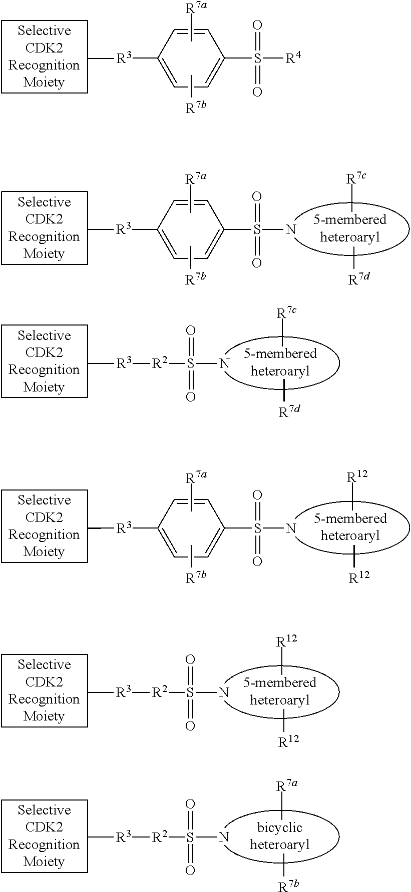

or a pharmaceutically acceptable salt thereof.

### Embodiments of Formula I′

In certain embodiments the heteroaryl sulfonyl compound of Formula I′ is selected from:

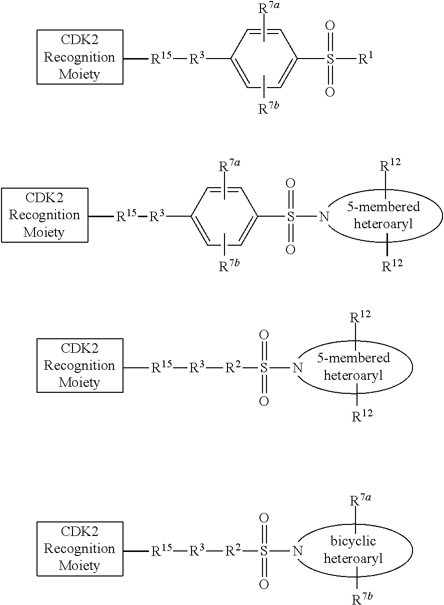

or a pharmaceutically acceptable salt thereof.

### Embodiments of Formula II′

In certain embodiments the heteroaryl sulfonyl compound of Formula II′ is selected from:

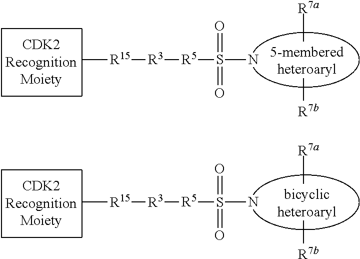

or a pharmaceutically acceptable salt thereof.

### Embodiments of Formula III′

In certain embodiments the heteroaryl sulfonyl compound of Formula III′ is selected from:

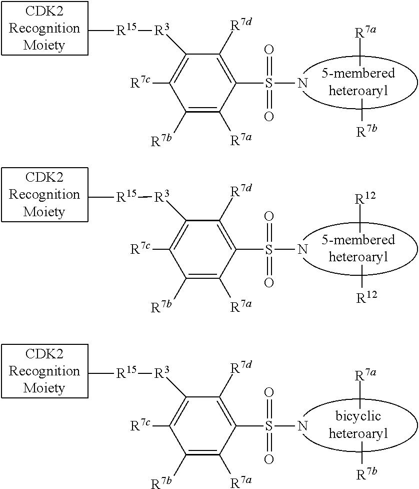

or a pharmaceutically acceptable salt thereof.

### Embodiments of Formula IV′

In certain embodiments the heteroaryl sulfonyl compound of Formula IV′ is selected from:

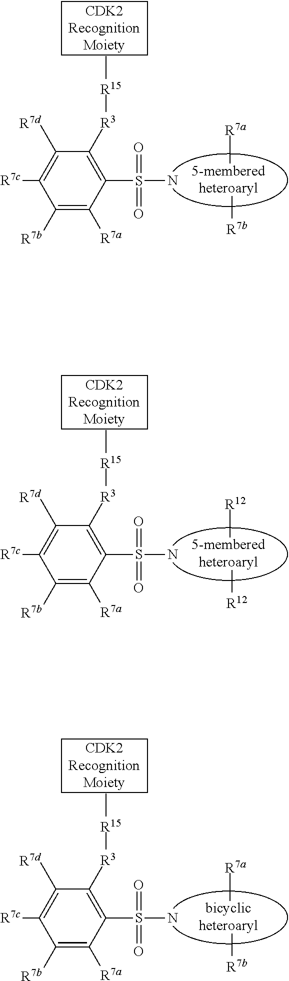

or a pharmaceutically acceptable salt thereof.

### Embodiments of Formula V′

In certain embodiments the heteroaryl sulfonyl compound of Formula V′ is selected from:

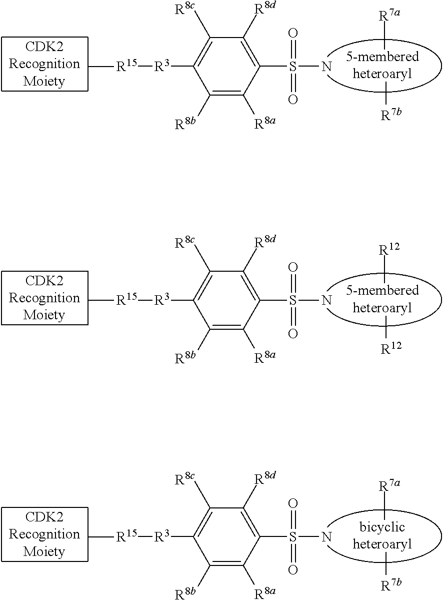

or a pharmaceutically acceptable salt thereof.

### Embodiments of Formula VI′

In certain embodiments the heteroaryl sulfonyl compound of Formula VI′ is selected from:

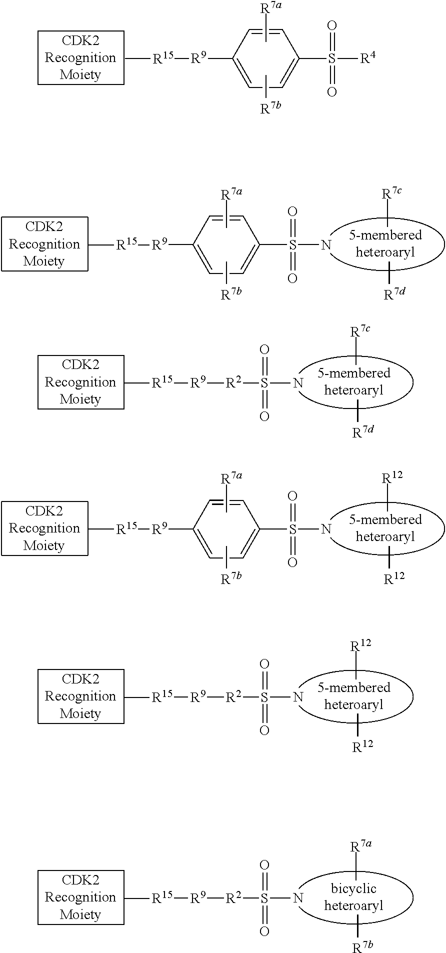

or a pharmaceutically acceptable salt thereof.

### Embodiments of Formula VII′

In certain embodiments the heteroaryl sulfonyl compound of Formula VII′ is selected

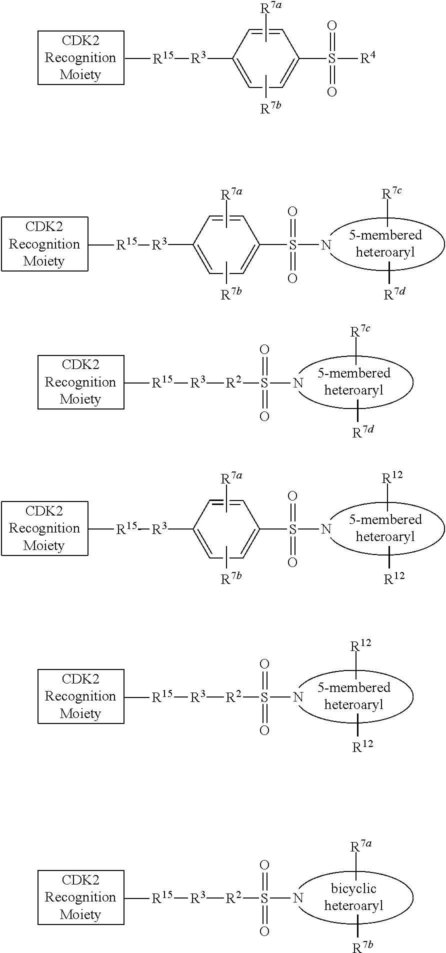

or a pharmaceutically acceptable salt thereof.

### Embodiments of Formula VIII′

In certain embodiments the heteroaryl sulfonyl compound of Formula VIII′ is selected from:

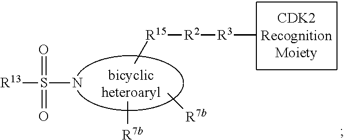

or a pharmaceutically acceptable salt thereof.

In certain embodiments the heteroaryl sulfonyl compound of Formula VIII′ is selected from:

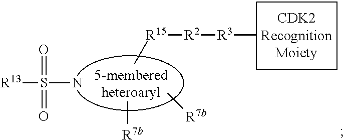

or a pharmaceutically acceptable salt thereof.

### Embodiments of Formula IX′

In certain embodiments the heteroaryl sulfonyl compound of Formula IX′ is selected from:

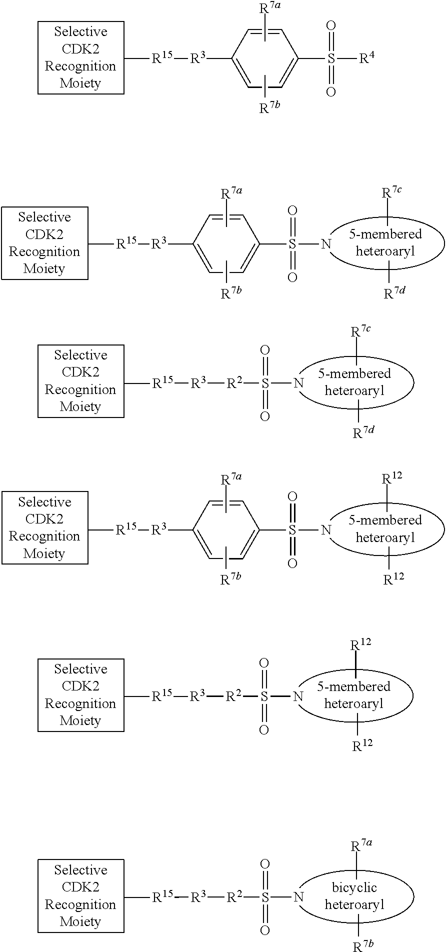

or a pharmaceutically acceptable salt thereof.

### Additional Embodiments

In certain embodiments the heteroaryl sulfonyl compound of the present invention is selected from:

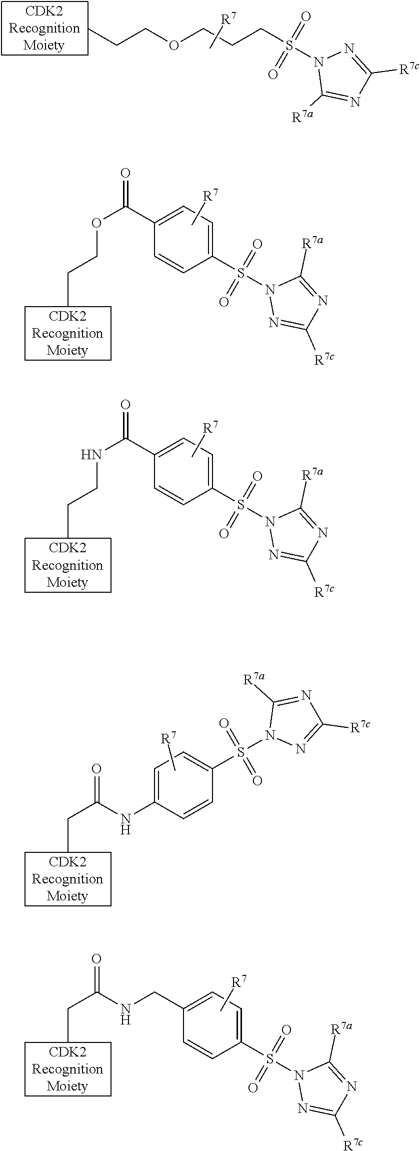

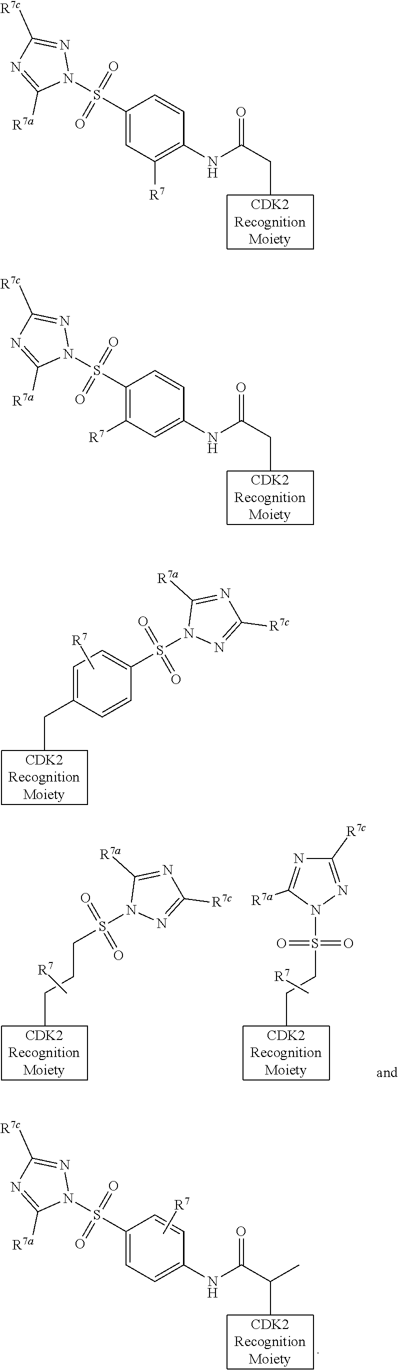

In certain embodiments the heteroaryl sulfonyl compound of the present invention is selected from:

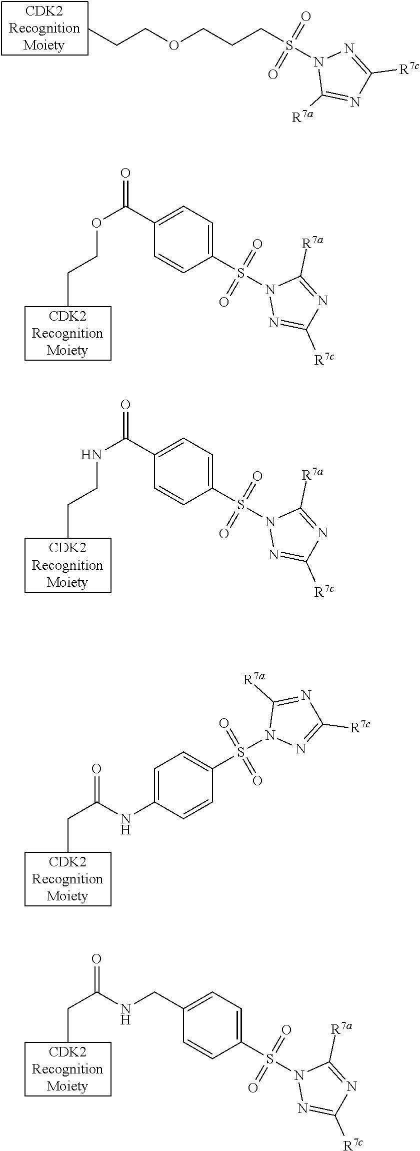

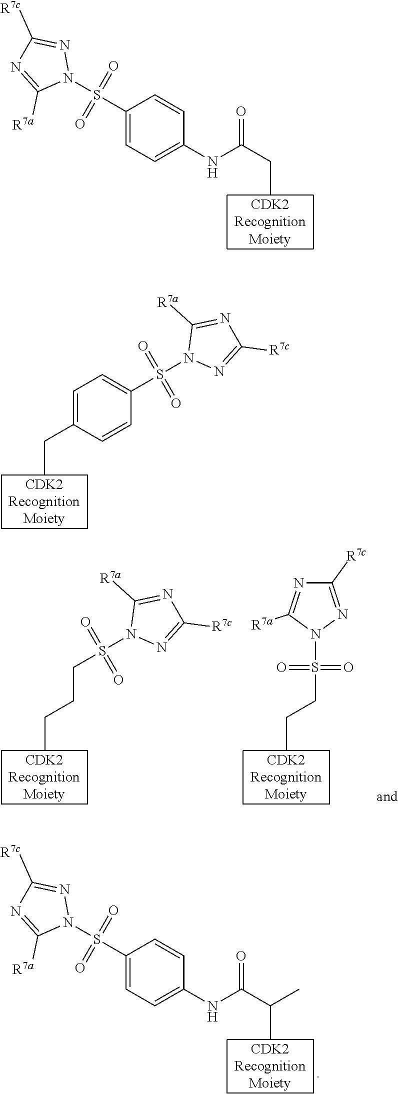

In certain embodiments the heteroaryl sulfonyl compound of the present invention is selected from:

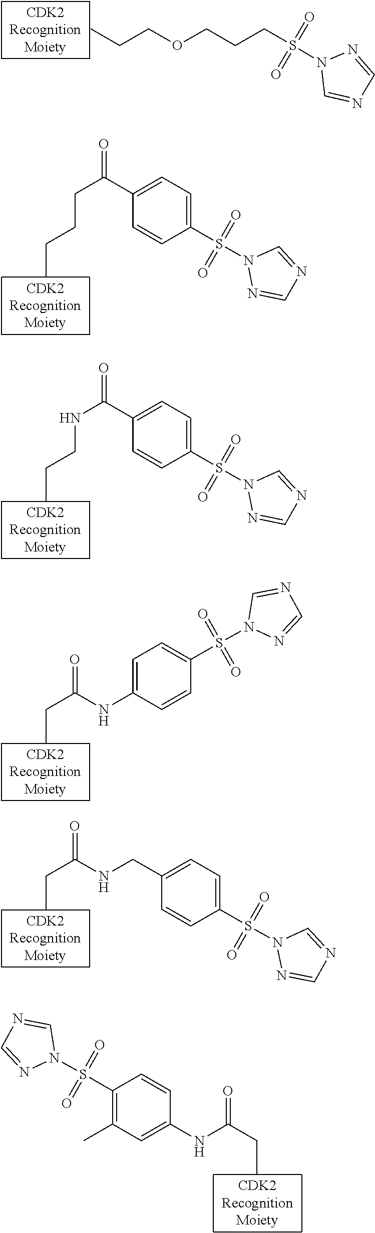 [SMILES: CCCOCCCS(=O)(=O)n1cncn1 ; CCCCC(=O)c1ccc(S(=O)(=O)n2cncn2)cc1 ; CCCNC(=O)c1ccc(S(=O)(=O)n2cncn2)cc1 ; CCC(=O)Nc1ccc(S(=O)(=O)n2cncn2)cc1 ; CCC(=O)NCc1ccc(S(=O)(=O)n2cncn2)cc1 ; CCC(=O)Nc1ccc(S(=O)(=O)n2cncn2)c(C)c1]

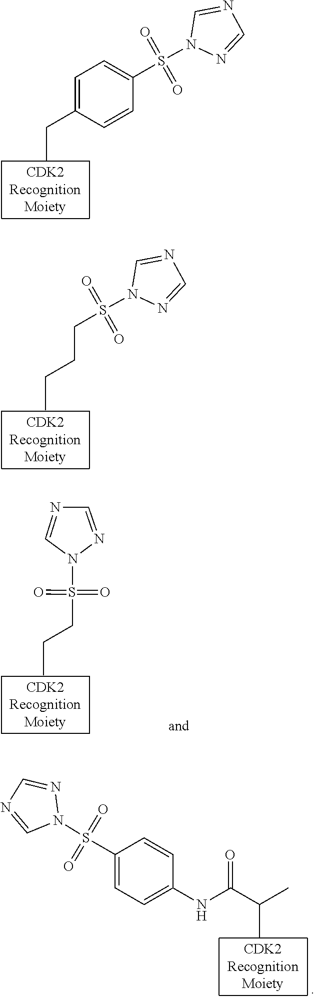 [SMILES: CCc1ccc(S(=O)(=O)n2cncn2)cc1 ; CCCCS(=O)(=O)n1cncn1 ; CCCS(=O)(=O)n1cncn1 ; CC(C)C(=O)Nc1ccc(S(=O)(=O)n2cncn2)cc1]

In certain embodiments the heteroaryl sulfonyl compound of the present invention is selected from:

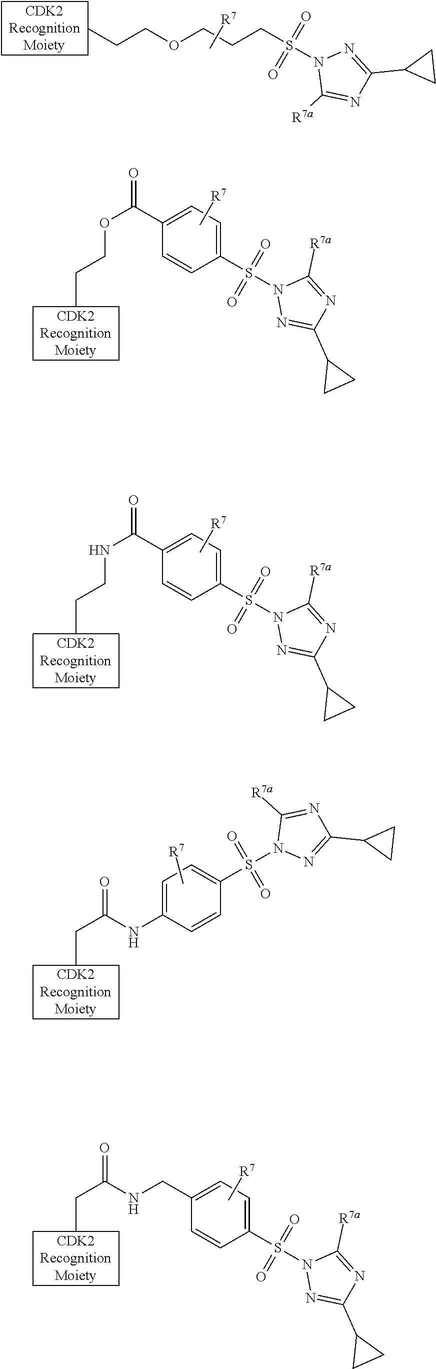

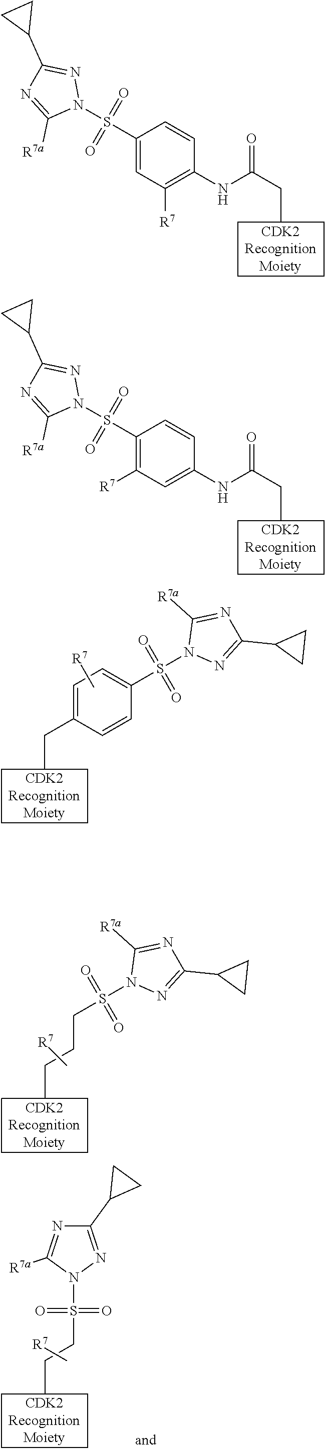

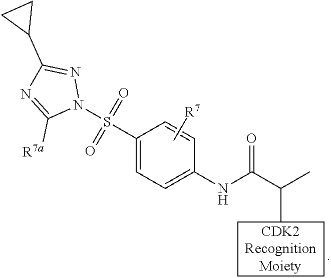

In certain embodiments the heteroaryl sulfonyl compound of the present invention is selected from:

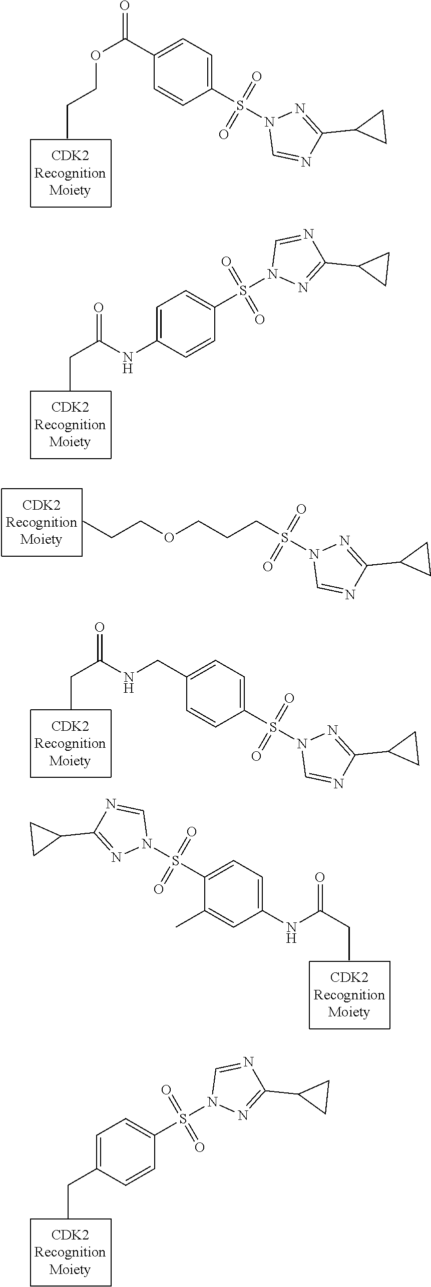 [SMILES: CCCOC(=O)c1ccc(S(=O)(=O)n2cnc(C3CC3)n2)cc1 ; CCC(=O)Nc1ccc(S(=O)(=O)n2cnc(C3CC3)n2)cc1 ; CCCOCCCS(=O)(=O)n1cnc(C2CC2)n1 ; CCC(=O)NCc1ccc(S(=O)(=O)n2cnc(C3CC3)n2)cc1 ; CCC(=O)Nc1ccc(S(=O)(=O)n2cnc(C3CC3)n2)c(C)c1 ; CCc1ccc(S(=O)(=O)n2cnc(C3CC3)n2)cc1]

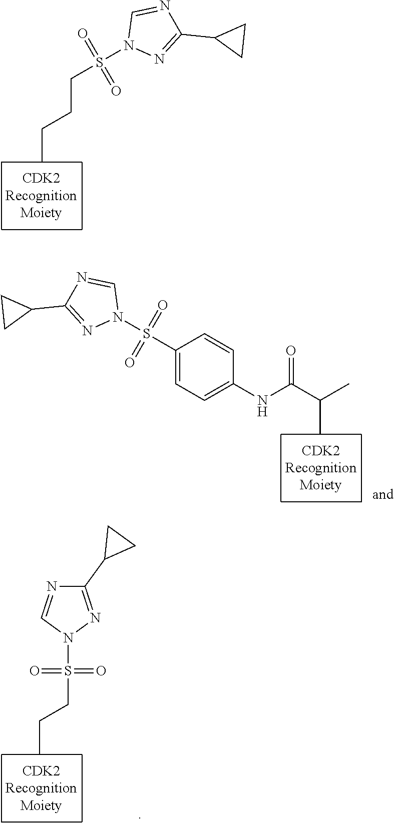 [SMILES: CCCCS(=O)(=O)n1cnc(C2CC2)n1 ; CC(C)C(=O)Nc1ccc(S(=O)(=O)n2cnc(C3CC3)n2)cc1 ; CCCS(=O)(=O)n1cnc(C2CC2)n1]

In certain embodiments the heteroaryl sulfonyl compound of the present invention is selected from:

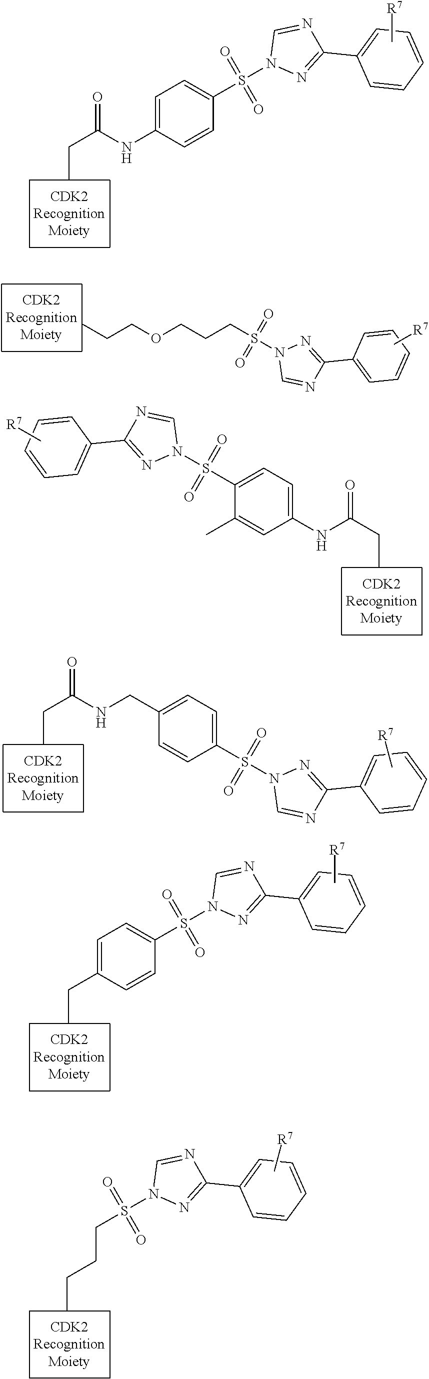

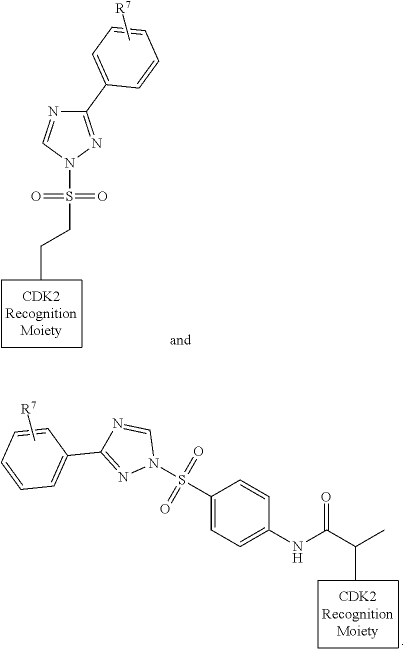

In certain embodiments the heteroaryl sulfonyl compound of the present invention is selected from:

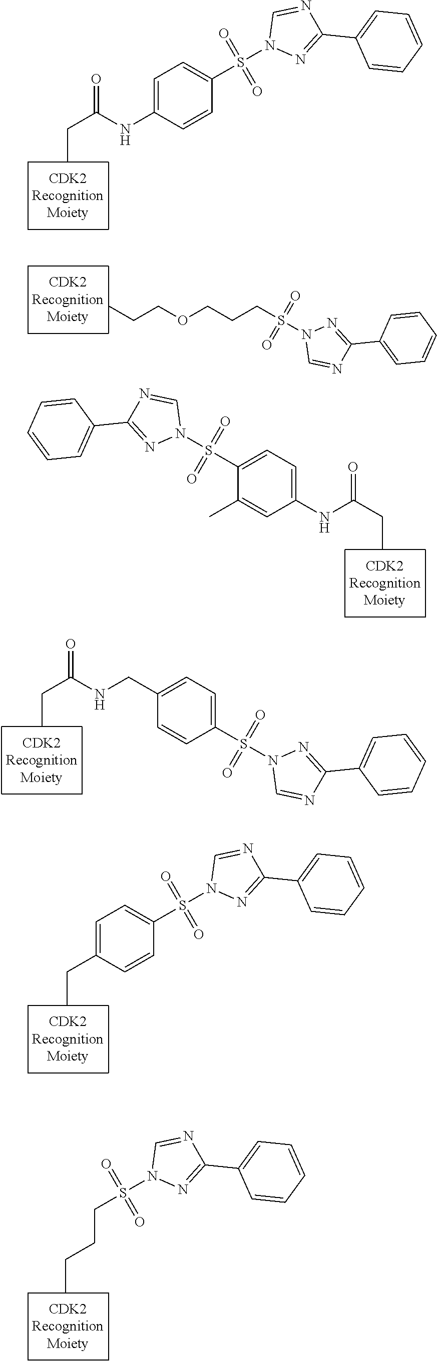 [SMILES: CCC(=O)Nc1ccc(S(=O)(=O)n2cnc(-c3ccccc3)n2)cc1 ; CCCOCCCS(=O)(=O)n1cnc(-c2ccccc2)n1 ; CCC(=O)Nc1ccc(S(=O)(=O)n2cnc(-c3ccccc3)n2)c(C)c1 ; CCC(=O)NCc1ccc(S(=O)(=O)n2cnc(-c3ccccc3)n2)cc1 ; CCc1ccc(S(=O)(=O)n2cnc(-c3ccccc3)n2)cc1 ; CCCCS(=O)(=O)n1cnc(-c2ccccc2)n1]

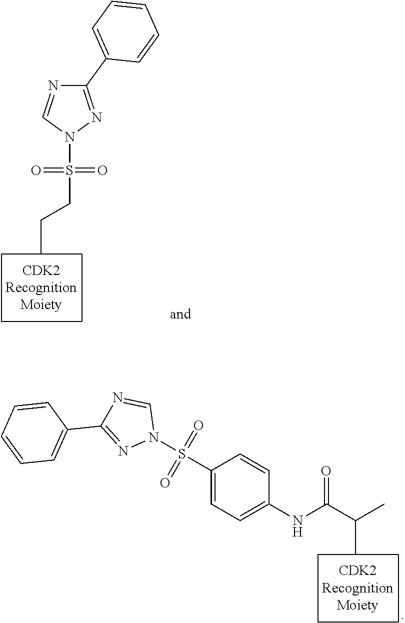 [SMILES: CCCS(=O)(=O)n1cnc(-c2ccccc2)n1 ; CC(C)C(=O)Nc1ccc(S(=O)(=O)n2cnc(-c3ccccc3)n2)cc1]

In certain embodiments the heteroaryl sulfonyl compound of the present invention is selected from:

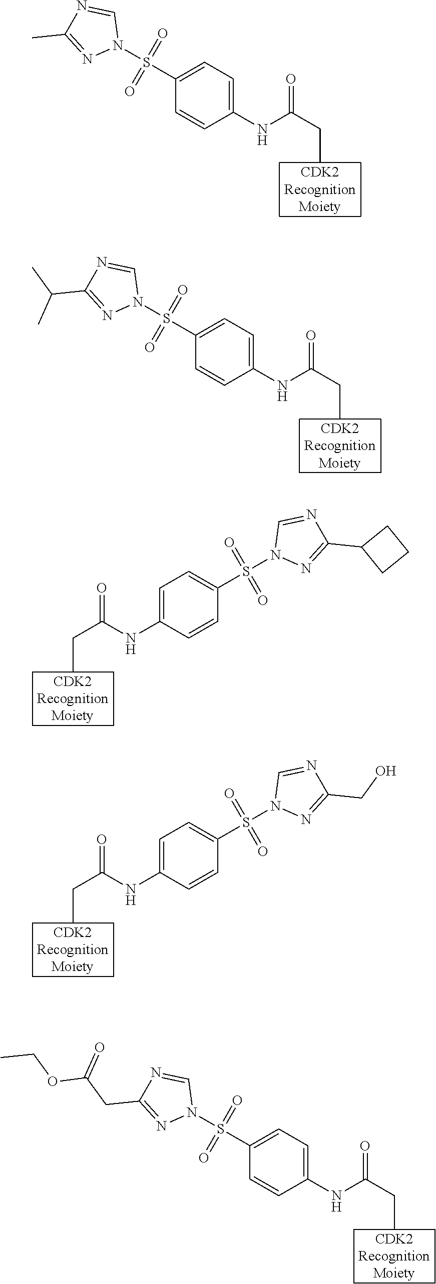 [SMILES: CCC(=O)Nc1ccc(S(=O)(=O)n2cnc(C)n2)cc1 ; CCC(=O)Nc1ccc(S(=O)(=O)n2cnc(C(C)C)n2)cc1 ; CCC(=O)Nc1ccc(S(=O)(=O)n2cnc(C3CCC3)n2)cc1 ; CCC(=O)Nc1ccc(S(=O)(=O)n2cnc(CO)n2)cc1 ; CCOC(=O)Cc1ncn(S(=O)(=O)c2ccc(NC(=O)CC)cc2)n1]

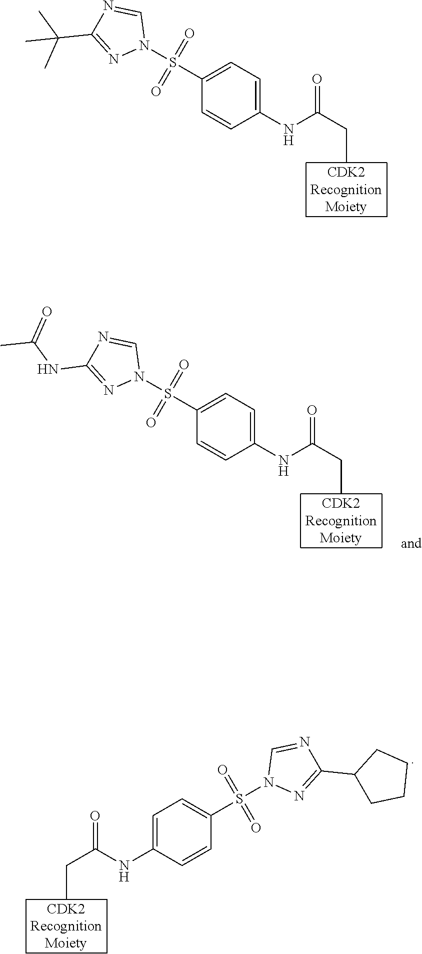 [SMILES: CCC(=O)Nc1ccc(S(=O)(=O)n2cnc(C(C)(C)C)n2)cc1 ; CCC(=O)Nc1ccc(S(=O)(=O)n2cnc(NC(C)=O)n2)cc1 ; CCC(=O)Nc1ccc(S(=O)(=O)n2cnc(C3CCCC3)n2)cc1]

In certain embodiments, the heteroaryl sulfonyl compound of the present invention is selected from:

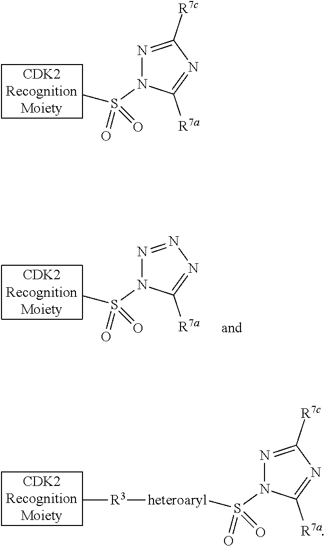

In certain embodiments, the heteroaryl sulfonyl compound of the present invention is selected from:

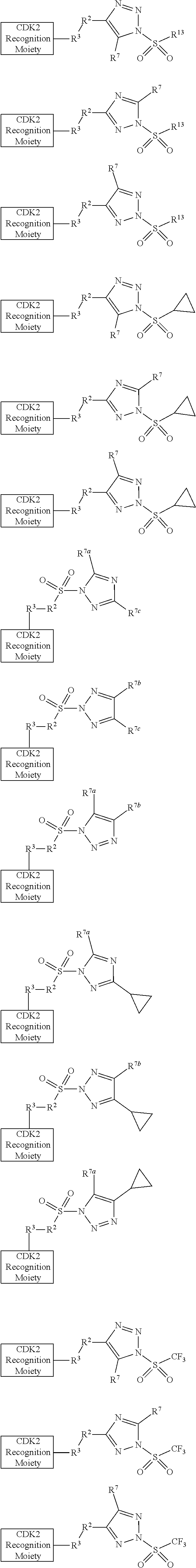

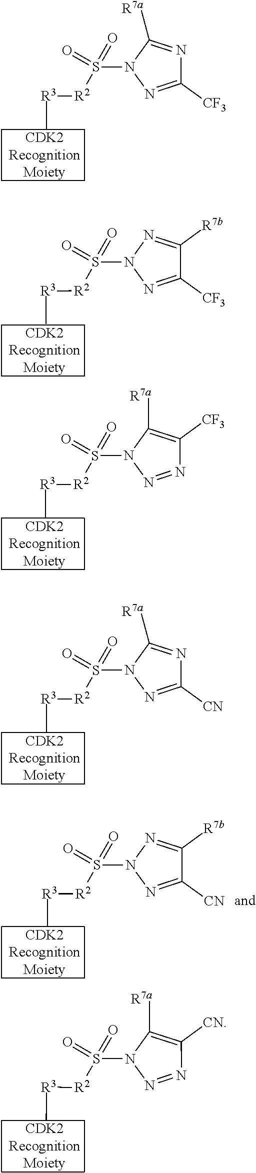

In certain embodiments, the heteroaryl sulfonyl compound of the present invention is selected from:

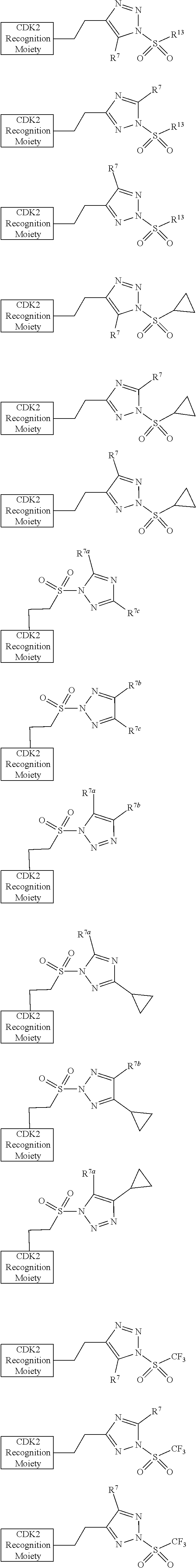

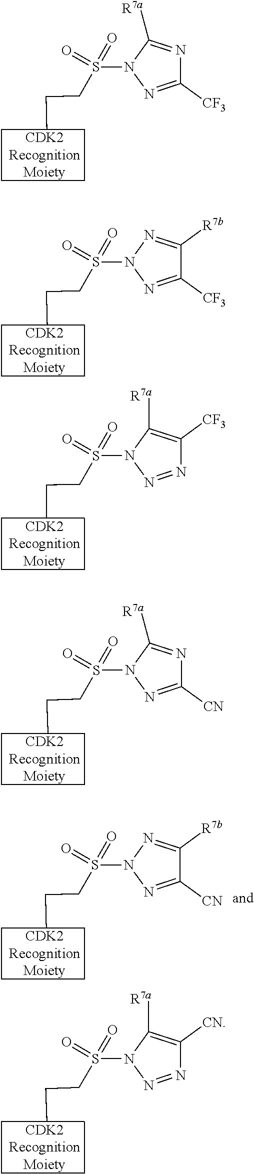

In certain embodiments, the heteroaryl sulfonyl compound of the present invention is selected from:

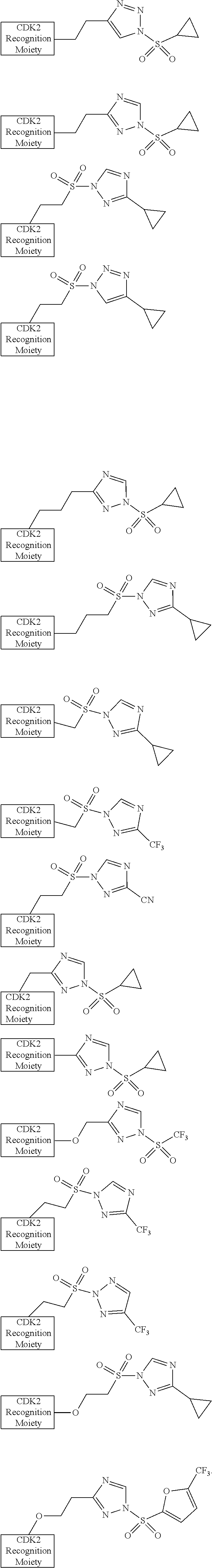

In certain embodiments, the heteroaryl sulfonyl compound of the present invention is selected from:

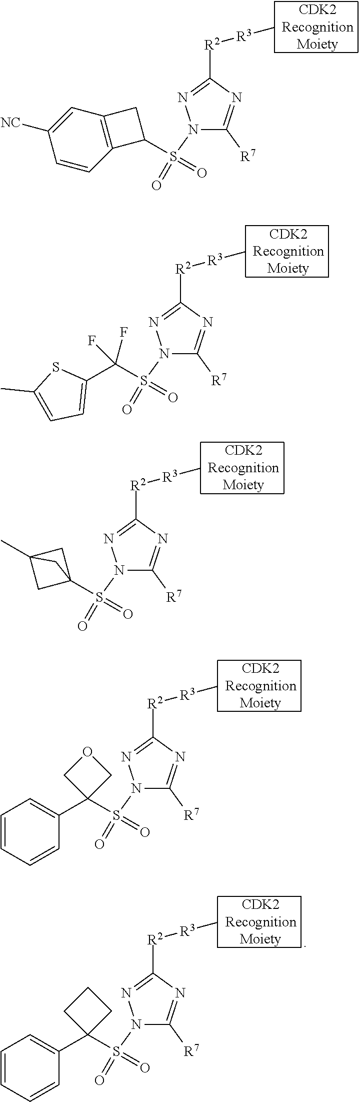

In certain embodiments, the heteroaryl sulfonyl compound of the present invention is selected from:

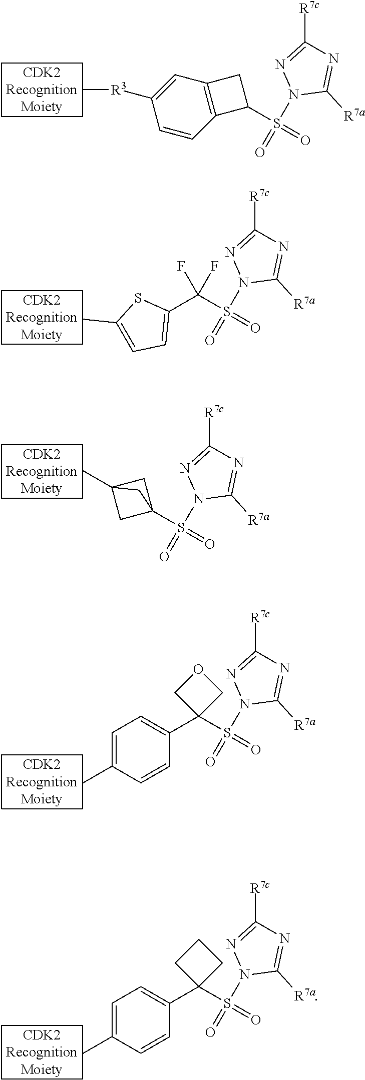

In certain embodiments, the heteroaryl sulfonyl compound of the present invention is selected from:

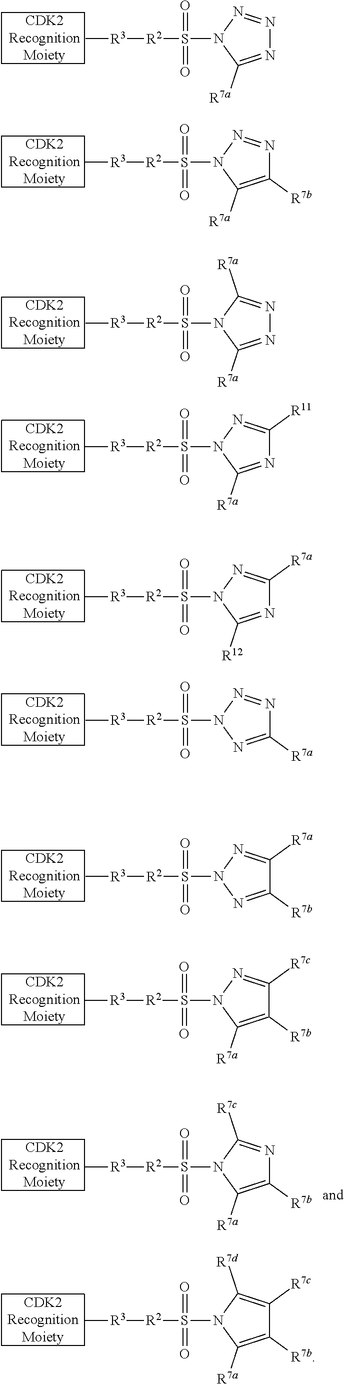

In certain embodiments, the heteroaryl sulfonyl compound of the present invention is selected from:

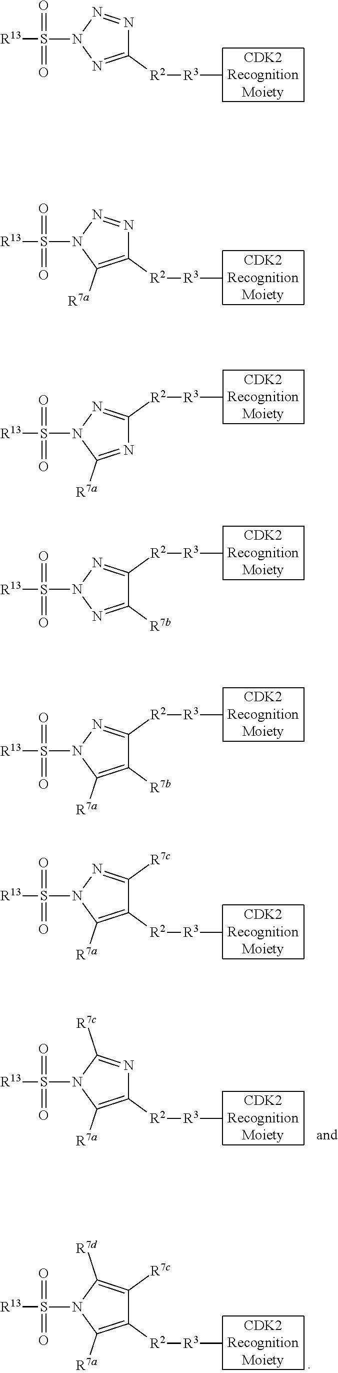

In certain embodiments, the heteroaryl sulfonyl compound of the present invention is selected from:

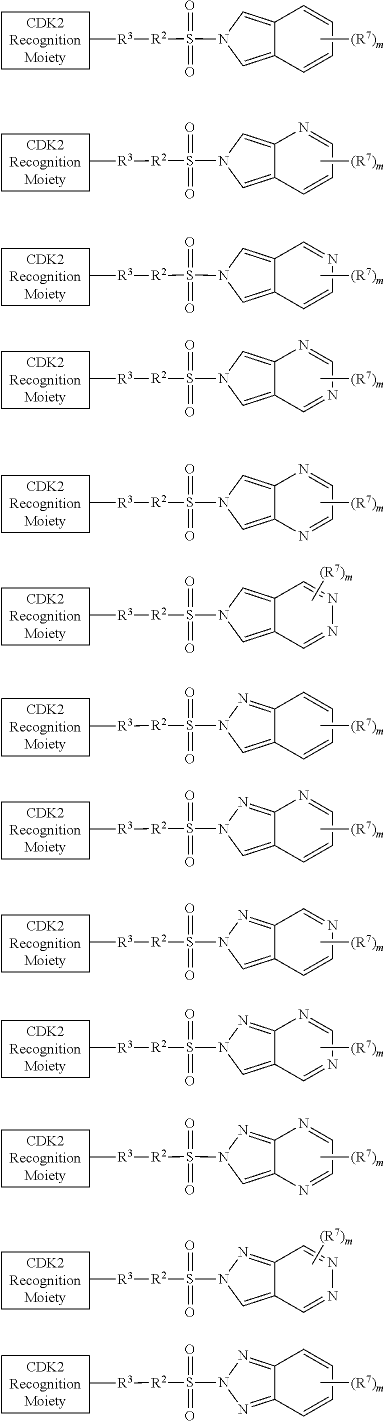

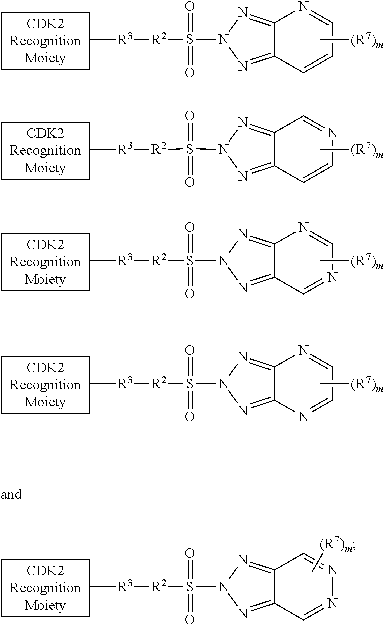

wherein m is independently selected from 1, 2, 3, and 4; and a floating bond on one ring of a bicyclic system means the substituent or substituents are optionally placed on any ring of the system.

For example,

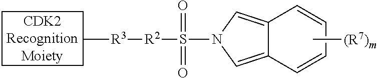

represents, but is not limited to:

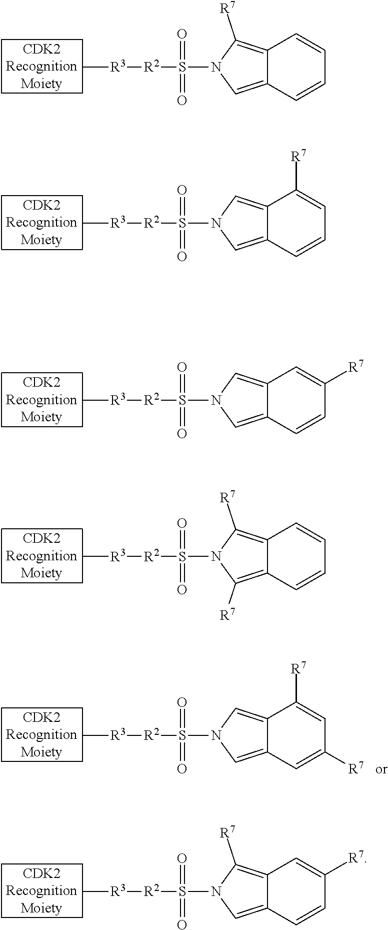

In certain embodiments, the heteroaryl sulfonyl compound of the present invention is selected from:

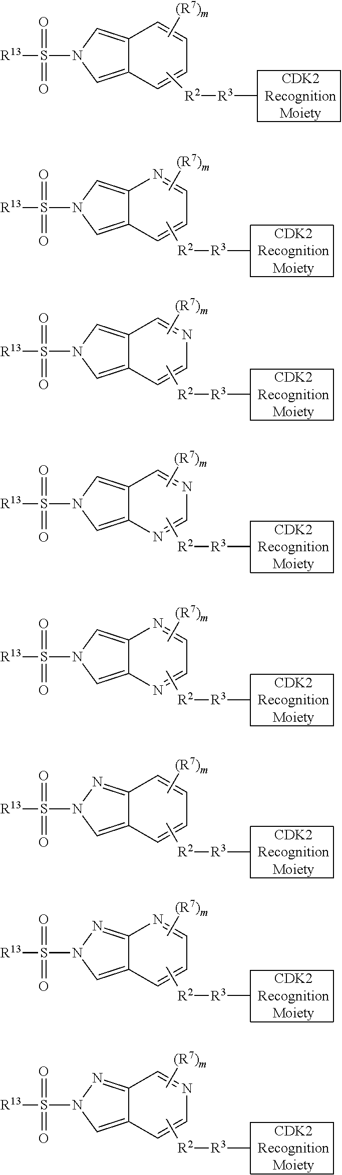

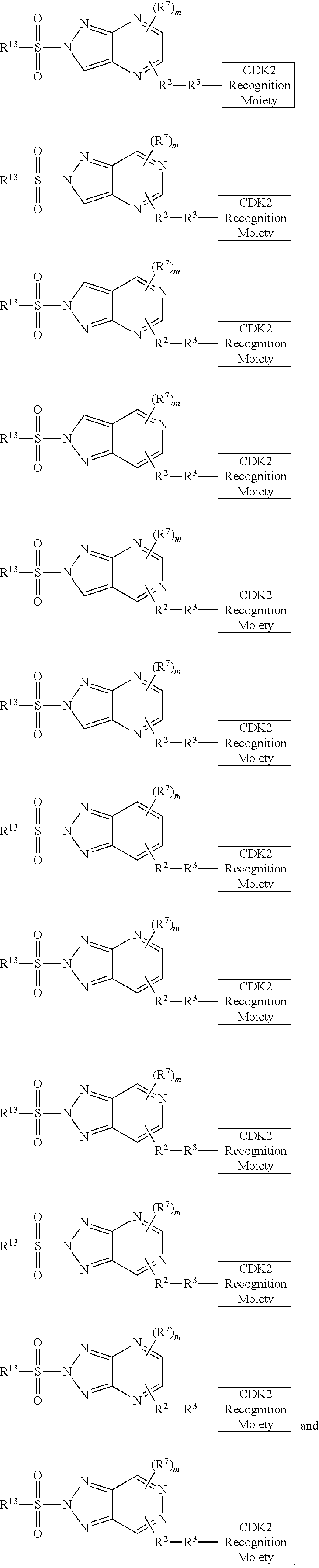

In certain embodiments, the heteroaryl sulfonyl compound of the present invention is selected from:

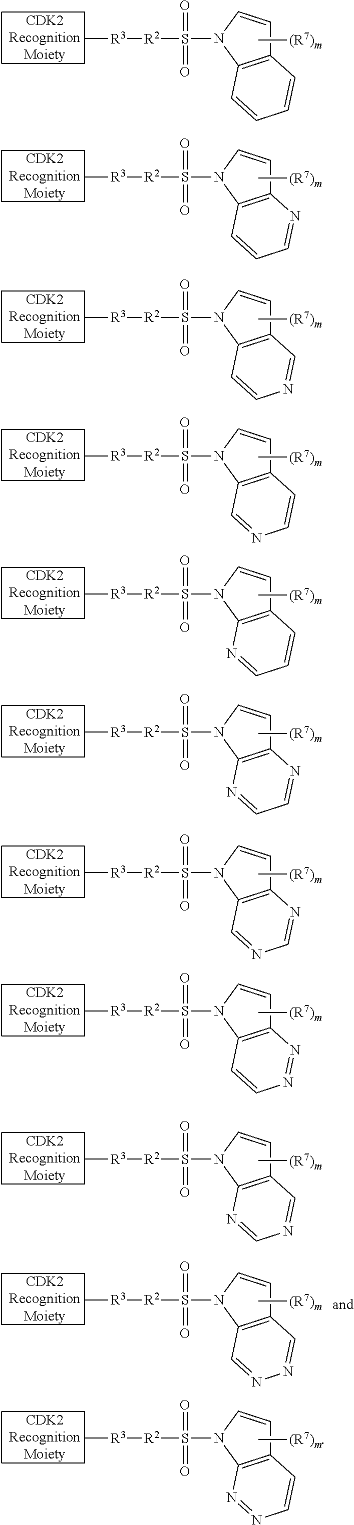

In certain embodiments, the heteroaryl sulfonyl compound of the present invention is selected from:

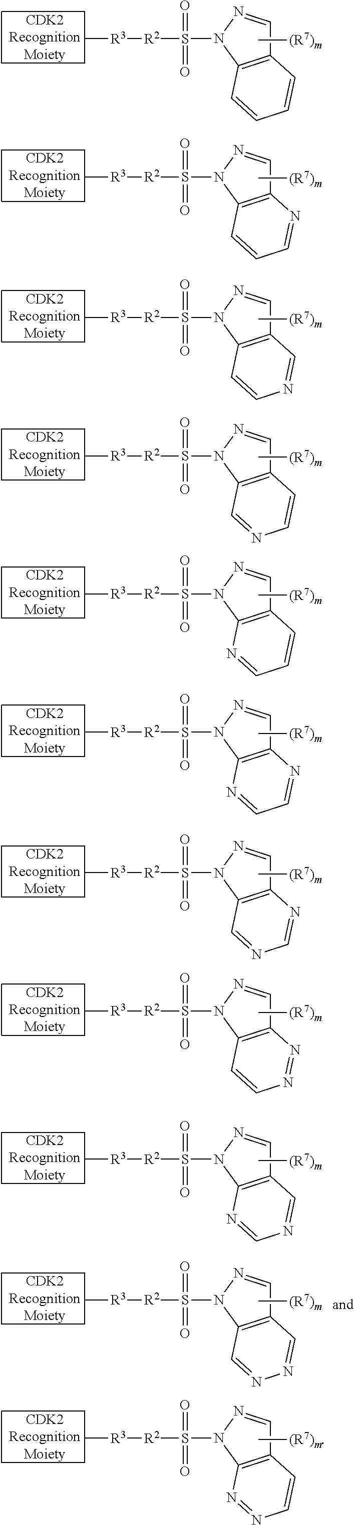

In certain embodiments, the heteroaryl sulfonyl compound of the present invention is selected from:

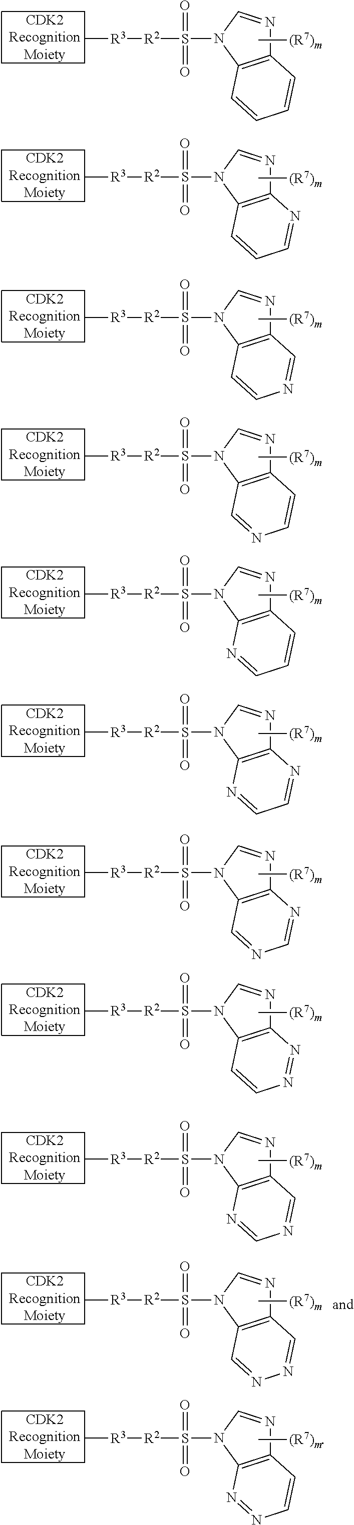

In certain embodiments, the heteroaryl sulfonyl compound of the present invention is selected from:

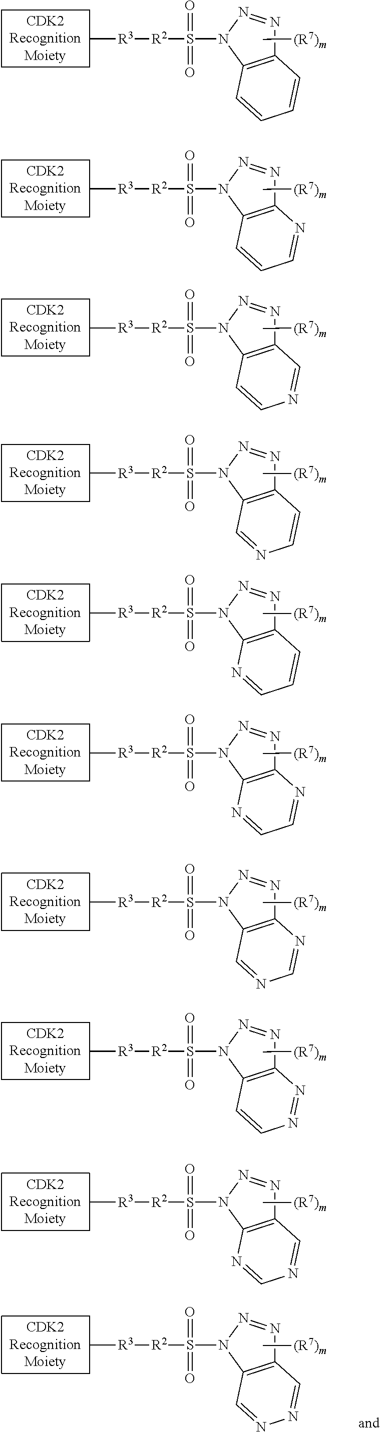

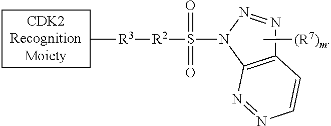

In certain embodiments, the heteroaryl sulfonyl compound of the present invention is selected from:

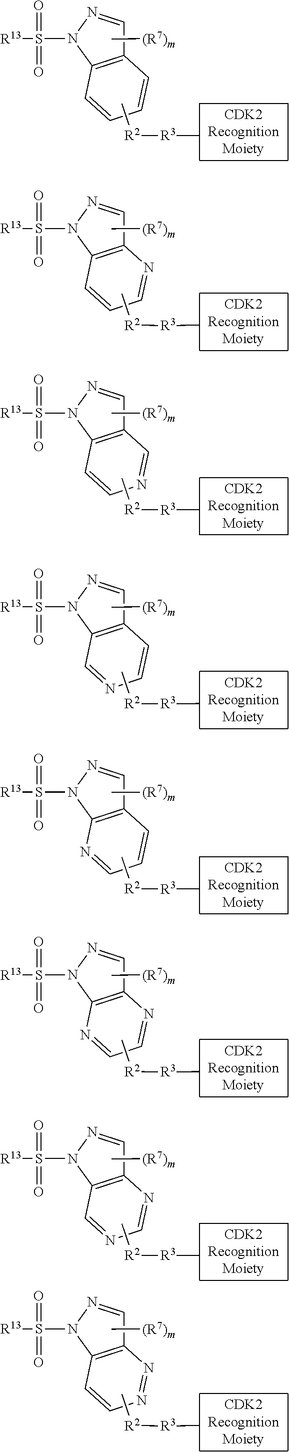

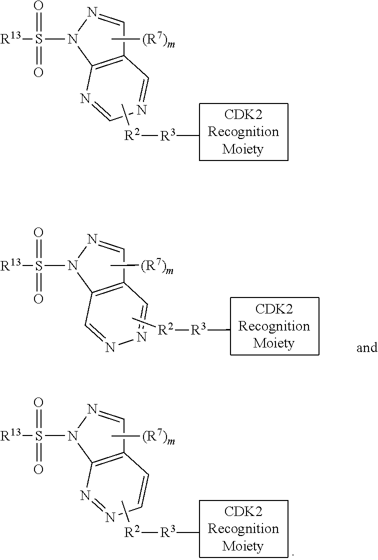

In certain embodiments, the heteroaryl sulfonyl compound of the present invention is selected from:

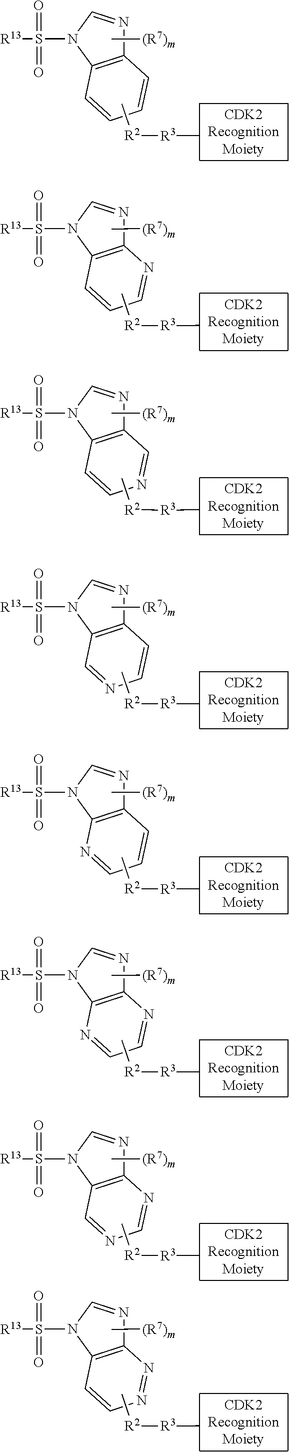

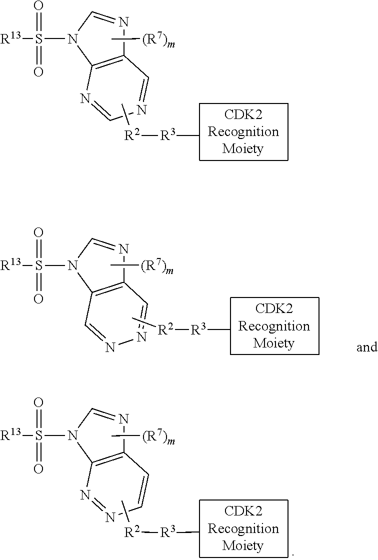

In certain embodiments, the heteroaryl sulfonyl compound of the present invention is selected from:

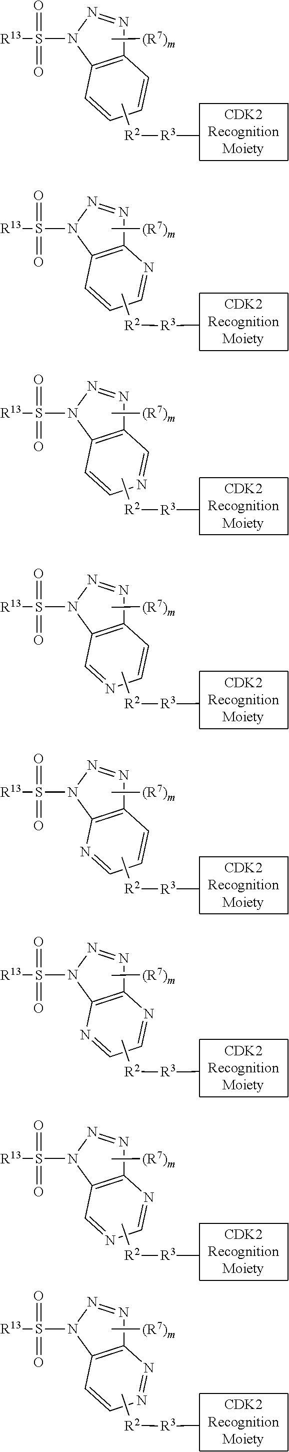

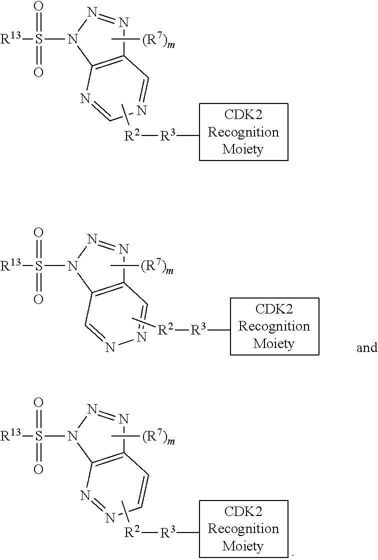

In certain embodiments, the heteroaryl sulfonyl compound of the present invention is selected from:

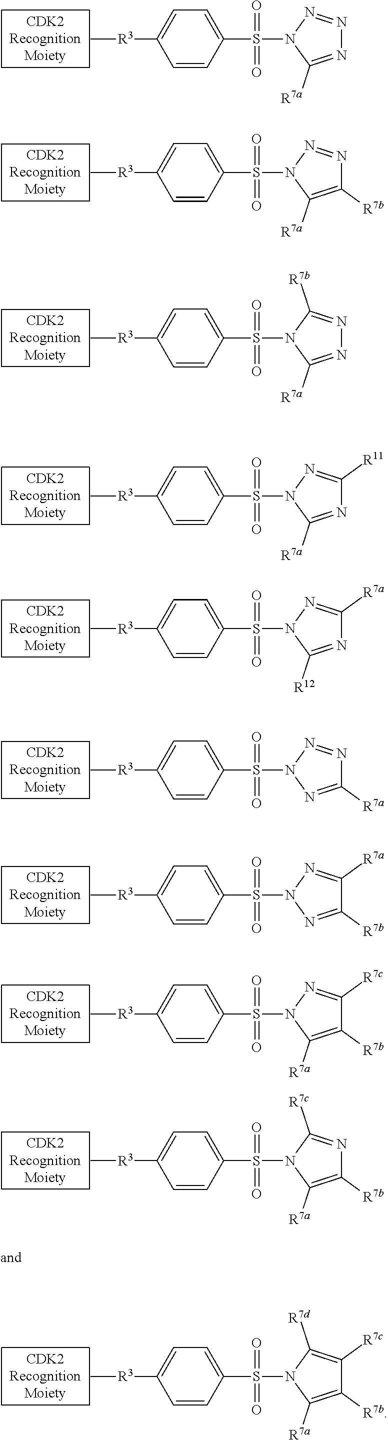

In certain embodiments, the heteroaryl sulfonyl compound of the present invention is selected from:

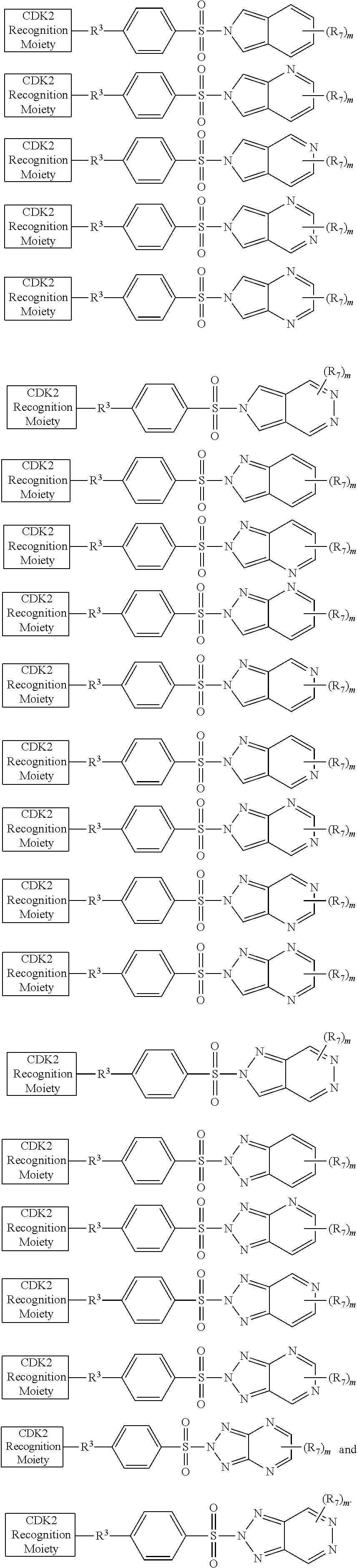

In certain embodiments, the heteroaryl sulfonyl compound of the present invention is selected from:

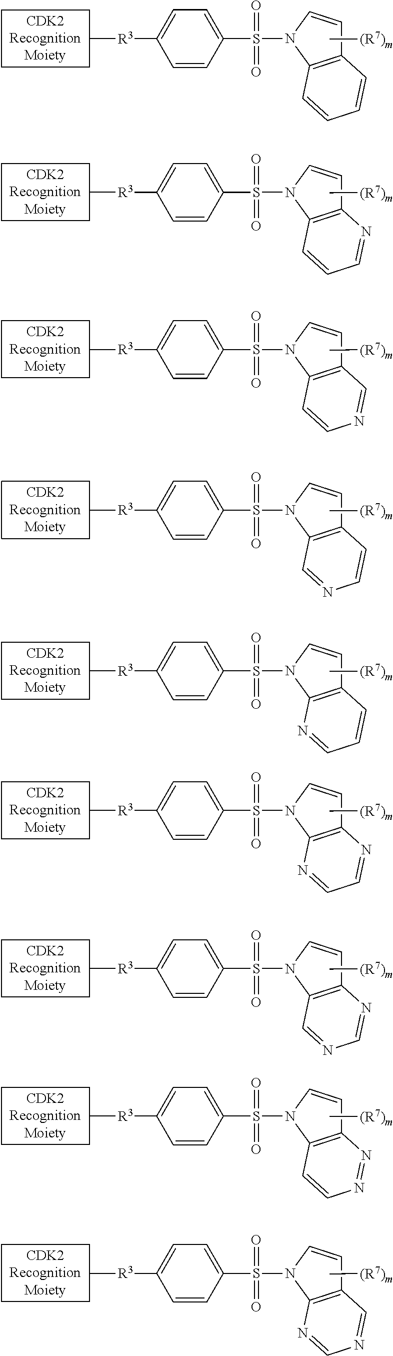

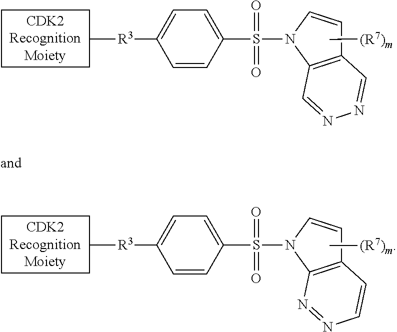

In certain embodiments, the heteroaryl sulfonyl compound of the present invention is selected from:

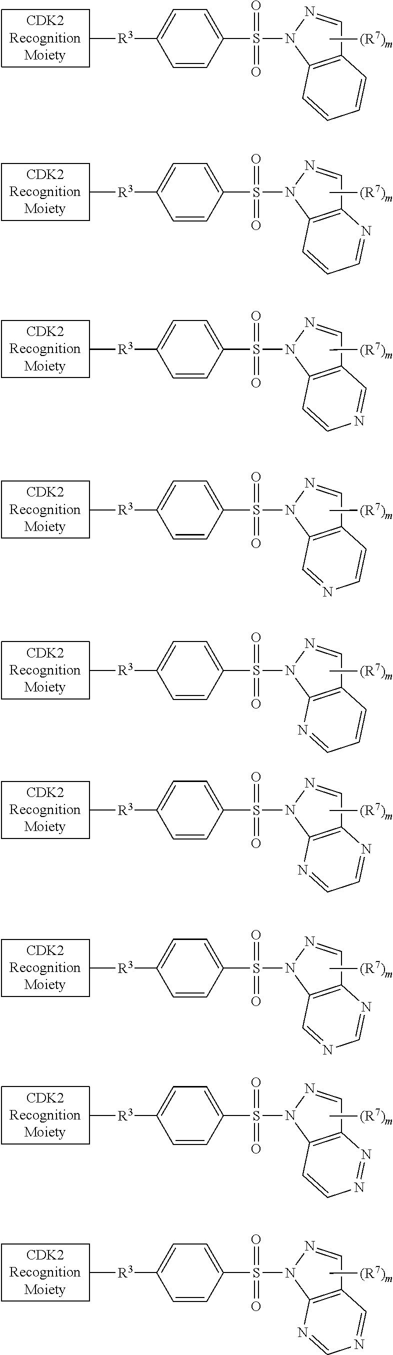

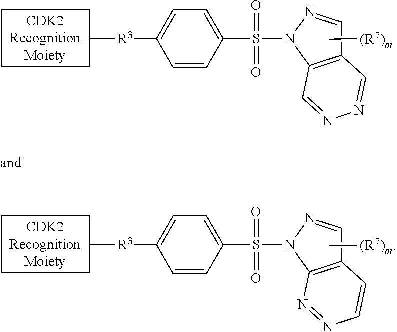

In certain embodiments, the heteroaryl sulfonyl compound of the present invention is selected from:

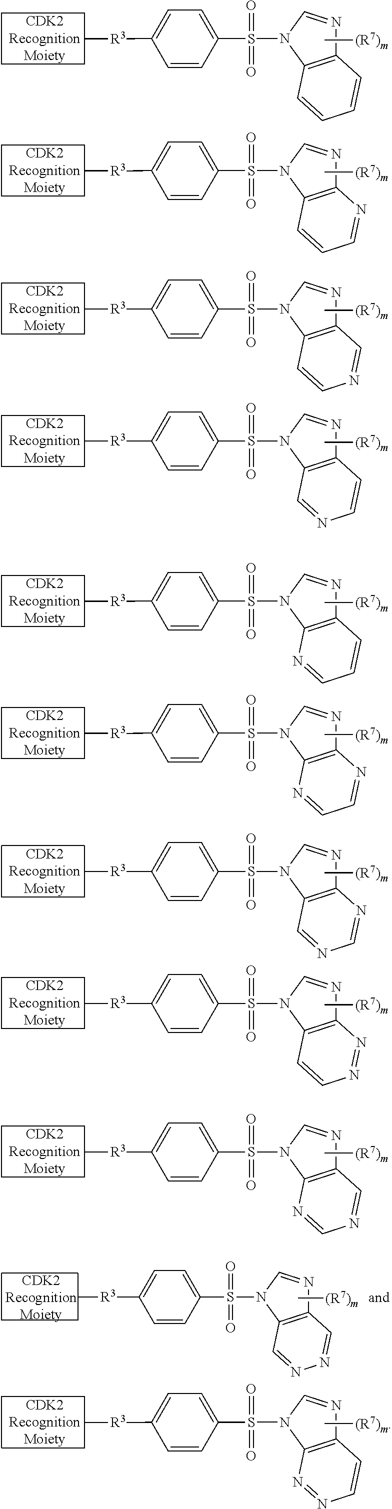

In certain embodiments, the heteroaryl sulfonyl compound of the present invention is selected from:

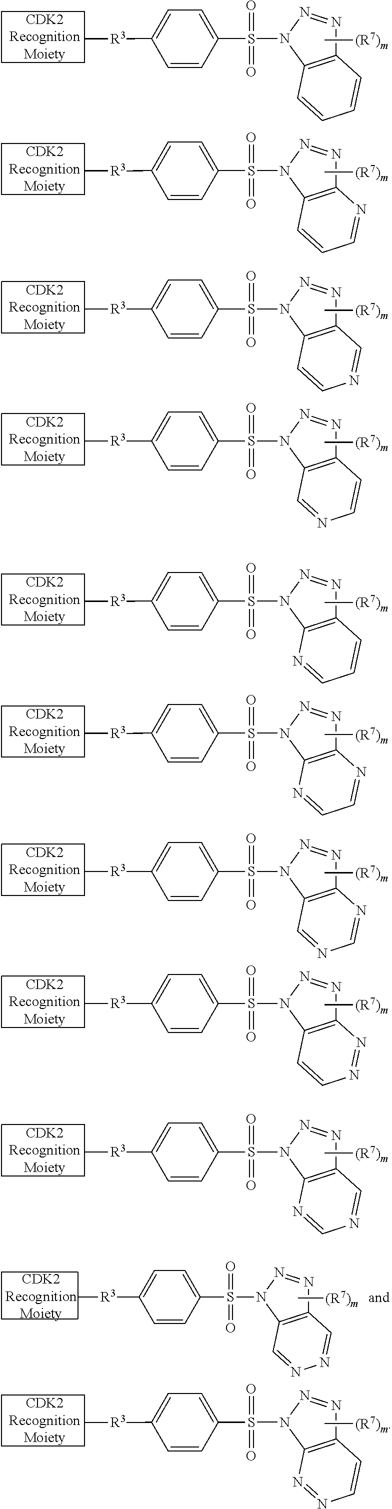

In certain embodiments, the heteroaryl sulfonyl compound of the present invention is selected from:

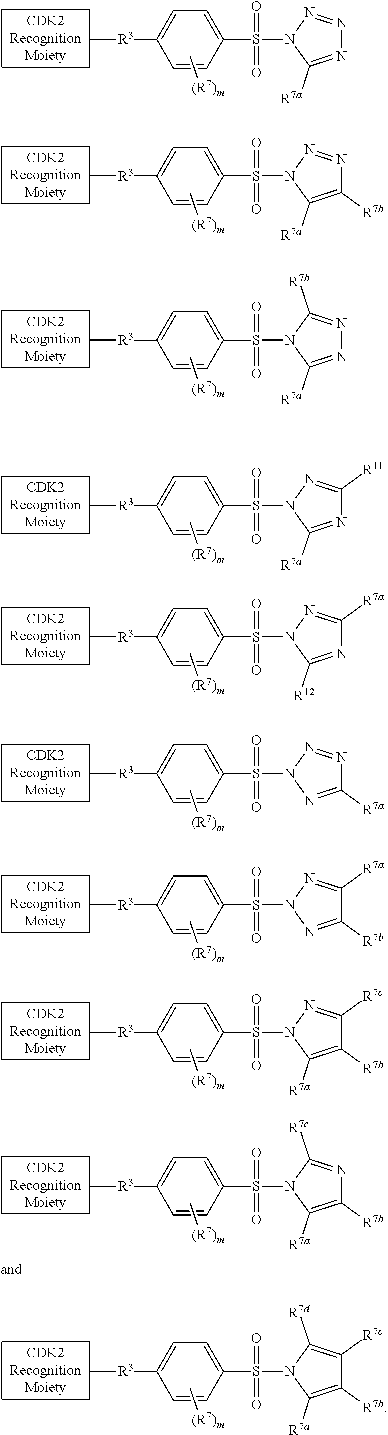

In certain embodiments, the heteroaryl sulfonyl compound of the present invention is selected from:

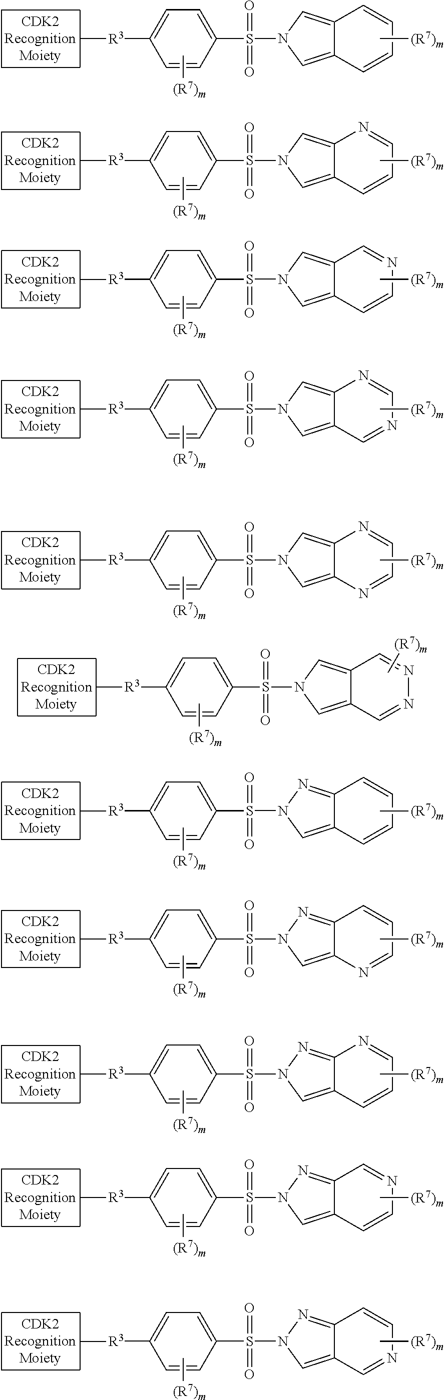

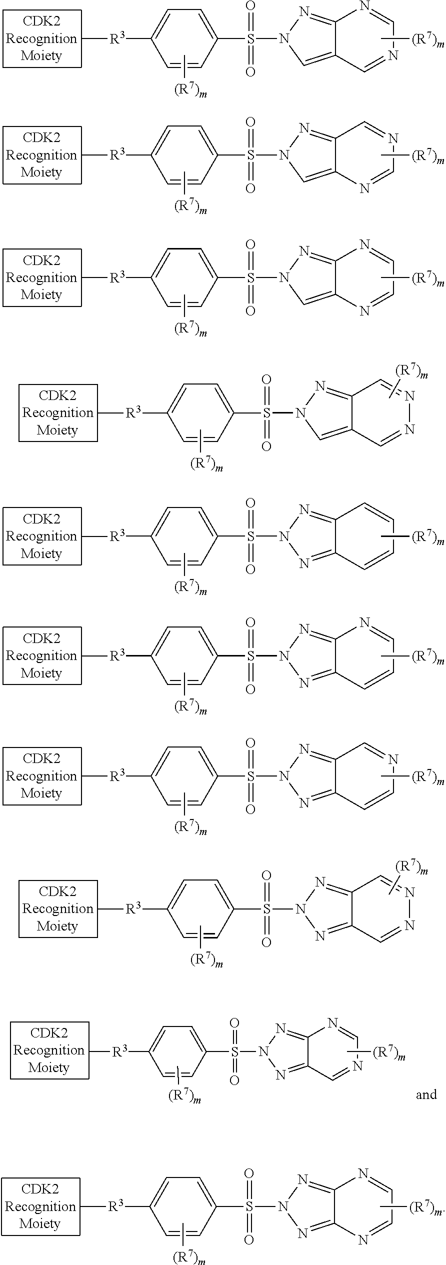

In certain embodiments, the heteroaryl sulfonyl compound of the present invention is selected from:

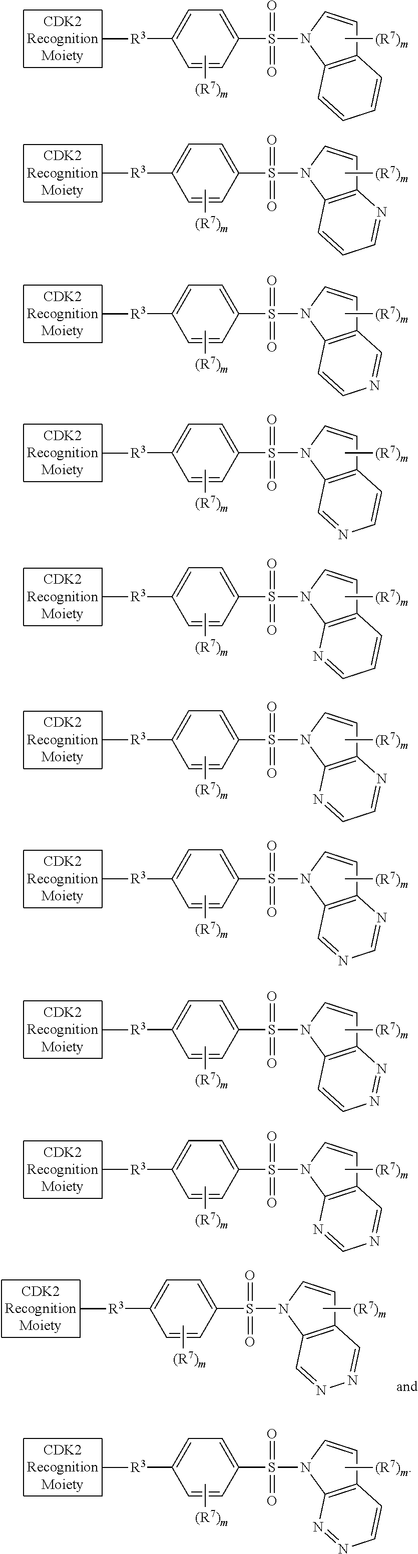

In certain embodiments, the heteroaryl sulfonyl compound of the present invention is selected from:

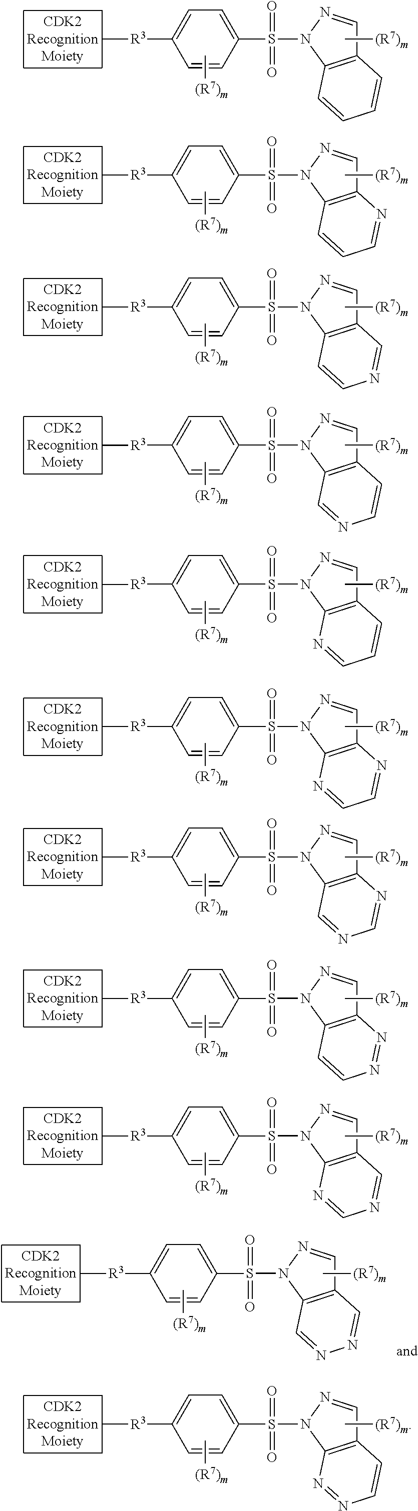

In certain embodiments, the heteroaryl sulfonyl compound of the present invention is selected from:

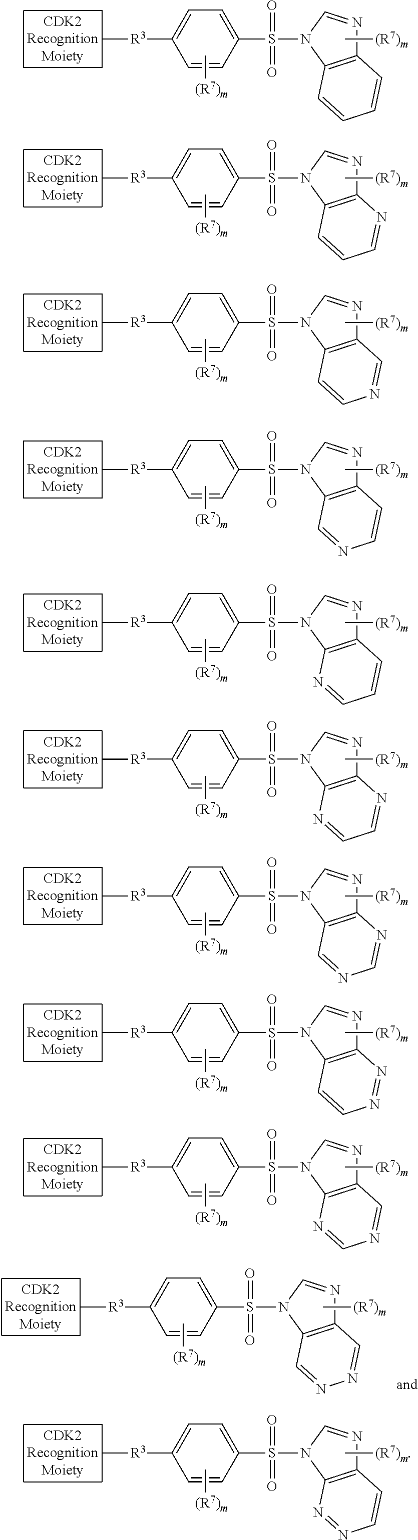

In certain embodiments, the heteroaryl sulfonyl compound of the present invention is selected from:

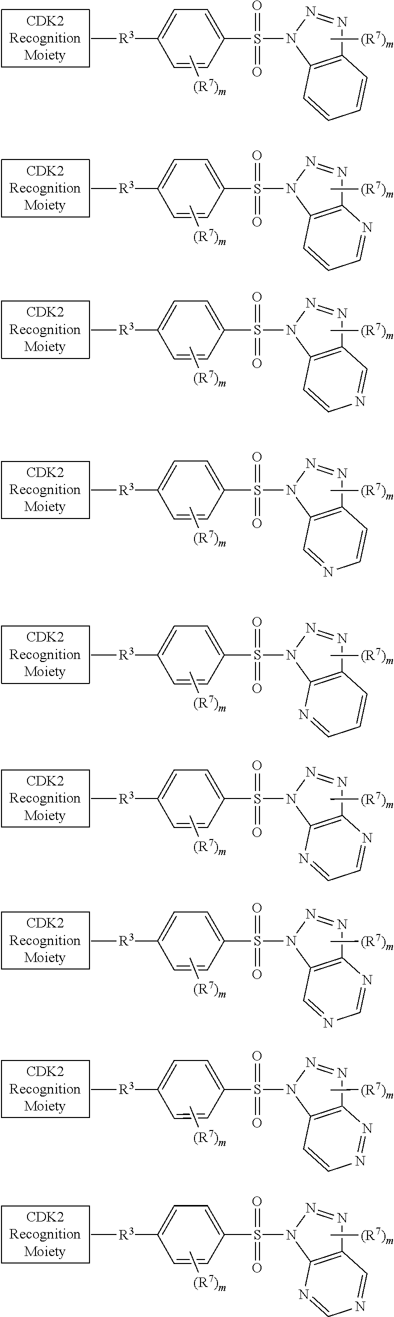

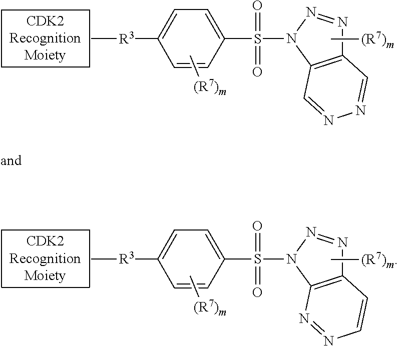

In certain embodiments, the heteroaryl sulfonyl compound of the present invention is selected from:

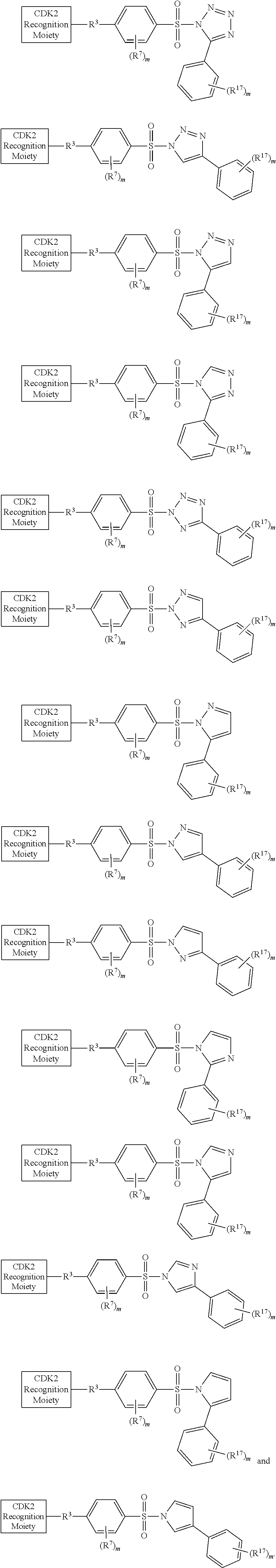

In certain embodiments, the heteroaryl sulfonyl compound of the present invention is selected from:

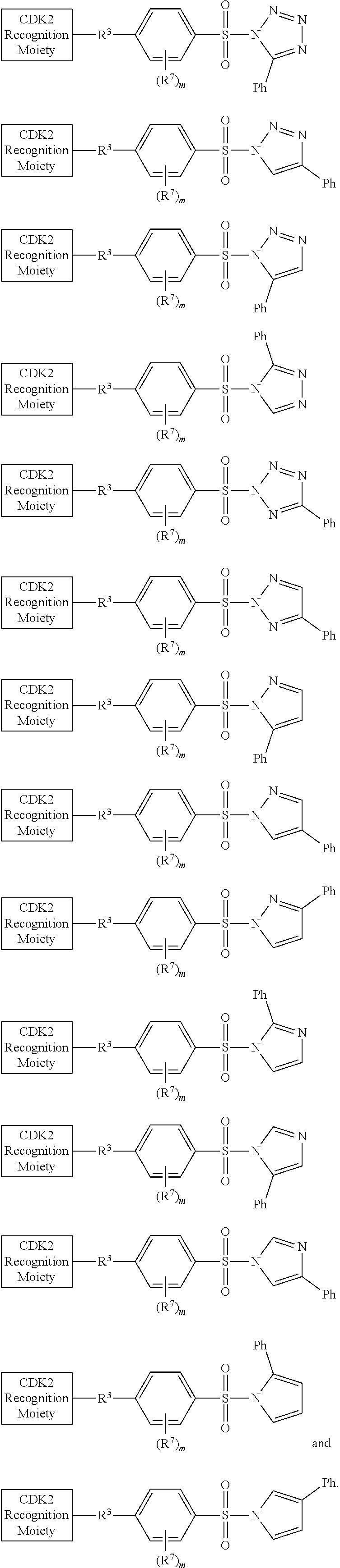

In certain embodiments, the heteroaryl sulfonyl compound of the present invention is

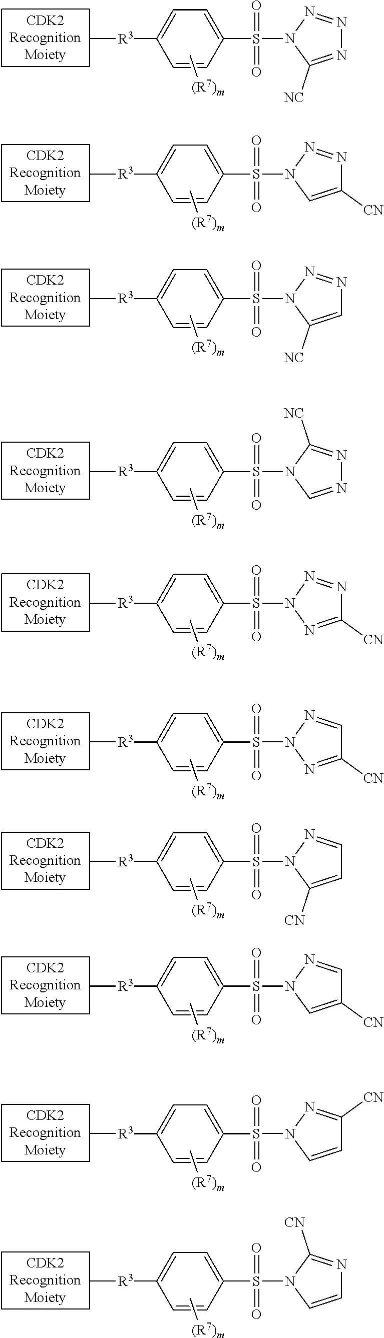

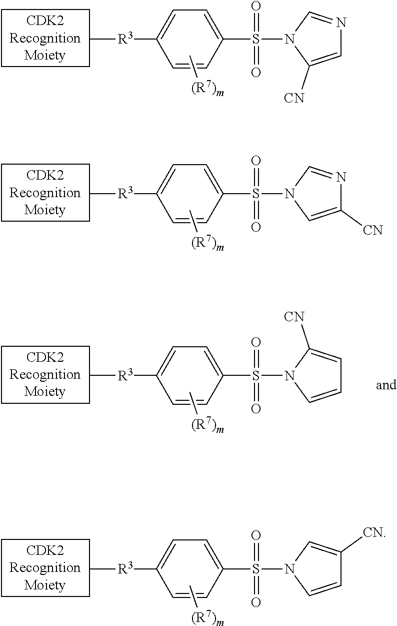

In certain embodiments, the heteroaryl sulfonyl compound of the present invention is selected from:

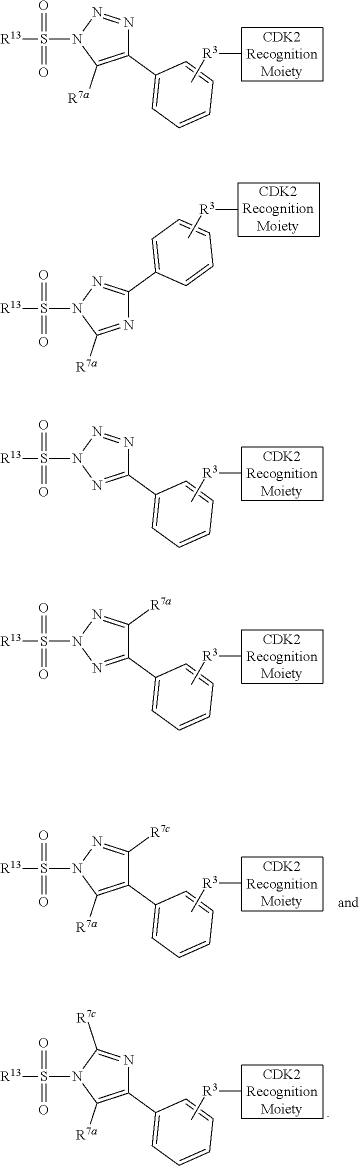

In certain embodiments, the heteroaryl sulfonyl compound of the present invention is selected from:

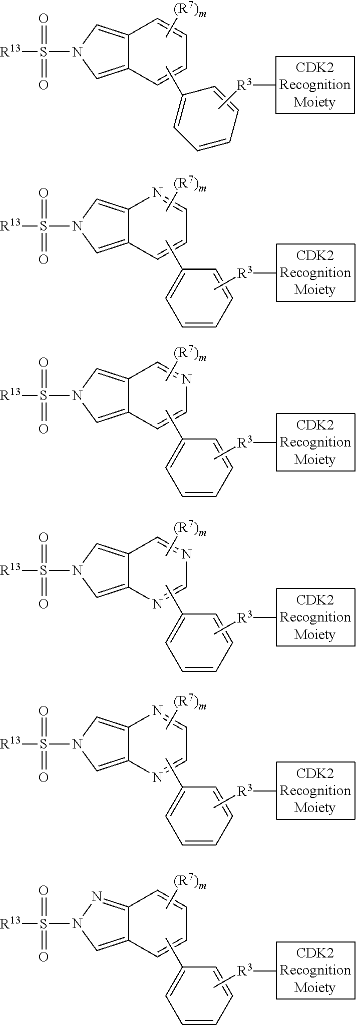

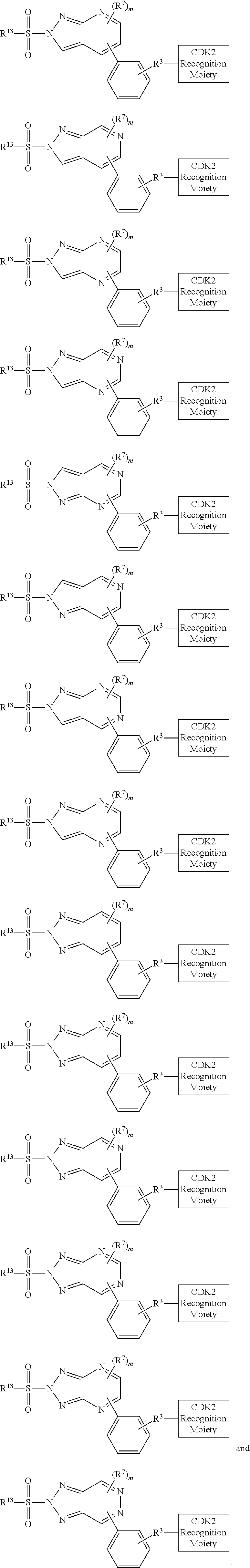

In certain embodiments, the heteroaryl sulfonyl compound of the present invention is selected from:

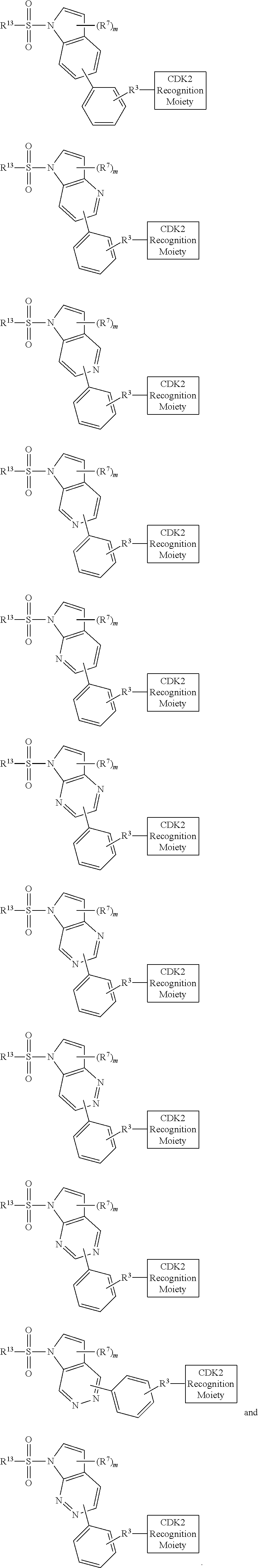

In certain embodiments, the heteroaryl sulfonyl compound of the present invention is selected from:

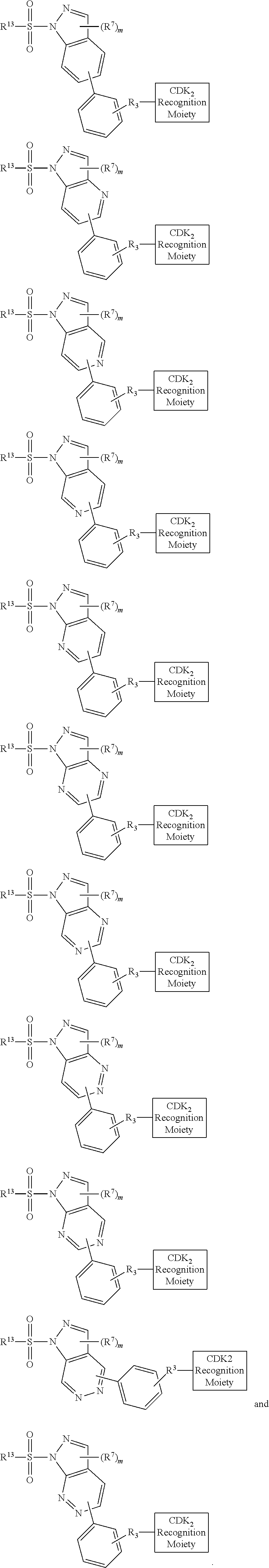

In certain embodiments, the heteroaryl sulfonyl compound of the present invention is selected from:

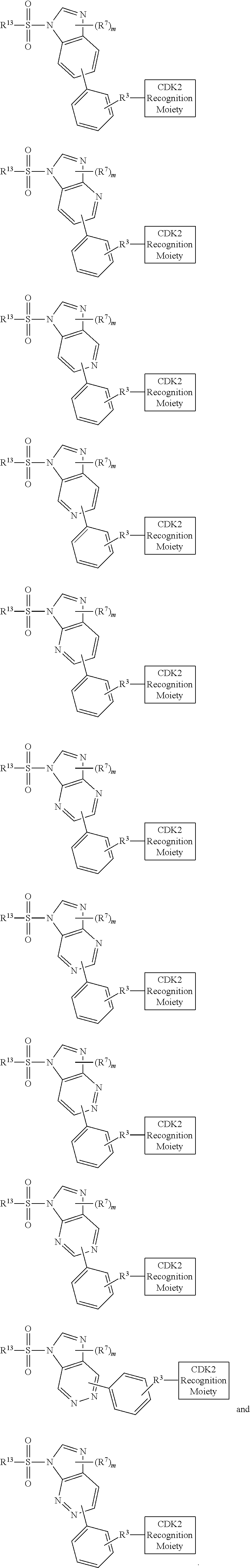

In certain embodiments, the heteroaryl sulfonyl compound of the present invention is selected from:

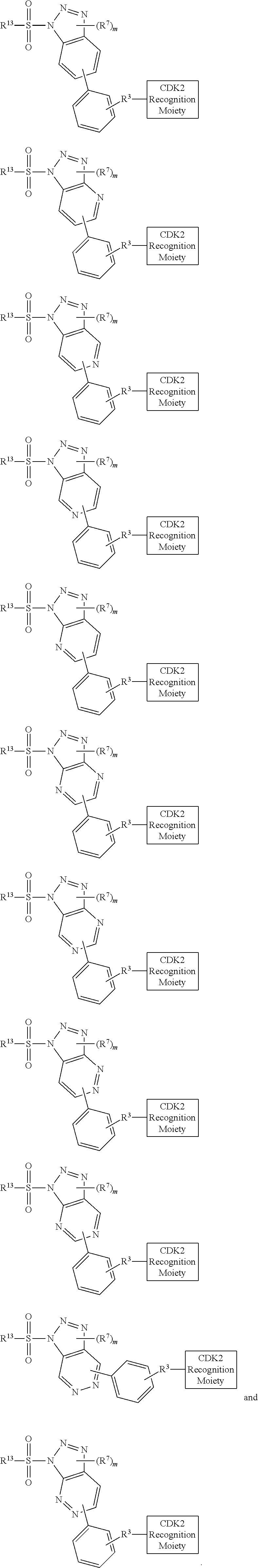

### Embodiments of R¹

In certain embodiments R¹ is

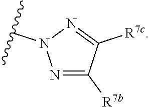

In certain embodiments R¹ is

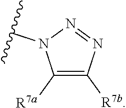

In certain embodiments R¹ is

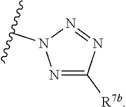

In certain embodiments R¹ is

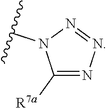

In certain embodiments R¹ is

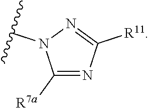

In certain embodiments R¹ is

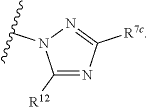

In certain embodiments R¹ is

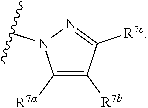

In certain embodiments R¹ is

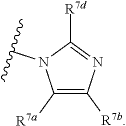

In certain embodiments R¹ is a fused bicyclic heteroaryl.

In certain embodiments R¹ or R⁴ is

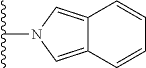 [SMILES: *n1cc2ccccc2c1]

optionally substituted with 1 2, 3, or 4 R⁷ substituents.

In certain embodiments R¹ or R⁴ is

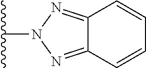 [SMILES: *n1nc2ccccc2n1]

optionally substituted with 1, 2, 3, or 4 R⁷ substituents.

In certain embodiments R¹ or R⁴ is

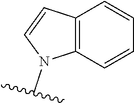 [SMILES: *n1ccc2ccccc21]

optionally substituted with 1, 2, 3, or 4 R⁷ substituents.

In certain embodiments R¹ or R⁴ is

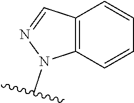 [SMILES: *n1ncc2ccccc21]

optionally substituted with 1, 2, 3, or 4 R⁷ substituents.

In certain embodiments R¹ or R⁴ is

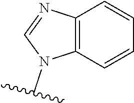 [SMILES: *n1cnc2ccccc21]

optionally substituted with 1, 2, 3, or 4 R⁷ substituents.

In certain embodiments R¹ or R⁴ is

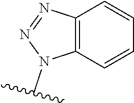 [SMILES: *n1nnc2ccccc21]

optionally substituted with 1, 2, 3, or 4 R⁷ substituents.

In certain embodiments R¹ or R⁴ is

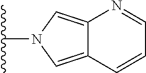 [SMILES: *n1cc2cccnc2c1]

optionally substituted with 1, 2, 3, or 4 R⁷ substituents.

In certain embodiments R¹ or R⁴ is

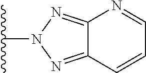 [SMILES: *n1nc2cccnc2n1]

optionally substituted with 1, 2, or 3 R⁷ substituents.

In certain embodiments R¹ or R⁴ is

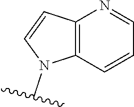 [SMILES: *n1ccc2ncccc21]

optionally substituted with 1, 2, 3, or 4 R⁷ substituents.

In certain embodiments R¹ or R⁴ is

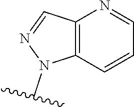 [SMILES: *n1ncc2ncccc21]

optionally substituted with 1, 2, 3, or 4 R⁷ substituents.

In certain embodiments R¹ or R⁴ is

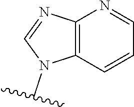 [SMILES: *n1cnc2ncccc21]

optionally substituted with 1, 2, 3, or 4 R⁷ substituents.

In certain embodiments R¹ or R⁴ is

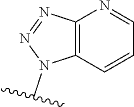 [SMILES: *n1nnc2ncccc21]

optionally substituted with 1, 2, or 3 R⁷ substituents.

In certain embodiments R¹ or R⁴ is

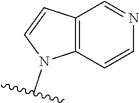 [SMILES: *n1ccc2cnccc21]

optionally substituted with 1, 2, 3, or 4 R⁷ substituents.

In certain embodiments R¹ or R⁴ is

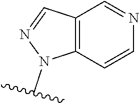 [SMILES: *n1ncc2cnccc21]

optionally substituted with 1, 2, 3, or 4 R⁷ substituents.

In certain embodiments R¹ or R⁴ is

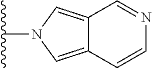 [SMILES: *n1cc2ccncc2c1]

optionally substituted with 1, 2, 3, or 4 R⁷ substituents.

In certain embodiments R¹ or R⁴ is

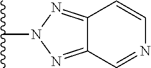 [SMILES: *n1nc2ccncc2n1]

optionality substituted with 1, 2, or 3 R⁷ substituents.

In certain embodiments R¹ or R⁴ is

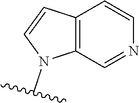 [SMILES: *n1ccc2ccncc21]

optionally substituted with 1, 2, 3, or 4 R⁷ substituents.

In certain embodiments R¹ or R⁴ is

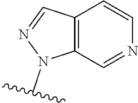 [SMILES: *n1ncc2ccncc21]

optionally substituted with 1, 2, 3, or 4 R⁷ substituents.

In certain embodiments R¹ or R⁴ is

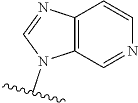 [SMILES: *n1cnc2ccncc21]

optionally substituted with 1, 2, 3, or 4 R⁷ substituents.

In certain embodiments R¹ or R⁴ is

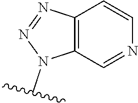 [SMILES: *n1nnc2ccncc21]

optionally substituted with 1, 2, or 3 R⁷ substituents.

In certain embodiments R¹ or R⁴ is

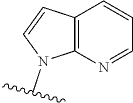 [SMILES: *n1ccc2cccnc21]

optionally substituted with 1, 2, 3, or 4 R⁷ substituents.

In certain embodiments R¹ or R⁴ is

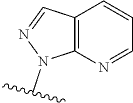 [SMILES: *n1ncc2cccnc21]

optionally substituted with 1, 2, 3, or 4 R⁷ substituents.

In certain embodiments R¹ or R⁴ is

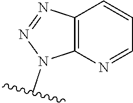 [SMILES: *n1nnc2cccnc21]

optionally substituted with 1, 2, or 3 R⁷ substituents.

In certain embodiments R¹ or R⁴ is

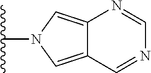 [SMILES: *n1cc2cncnc2c1]

optionally substituted with 1, 2, 3, or 4 R⁷ substituents.

In certain embodiments R¹ or R⁴ is

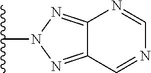 [SMILES: *n1nc2cncnc2n1]

optionally substituted with 1 or 2 R⁷ substituents.

In certain embodiments R¹ or R⁴ is

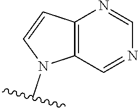 [SMILES: *n1ccc2ncncc21]

optionally substituted with 1, 2, 3, or 4 R⁷ substituents.

In certain embodiments R¹ or R⁴ is

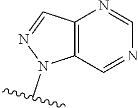 [SMILES: *n1ncc2ncncc21]

optionally substituted with 1, 2, or 3 R⁷ substituents.

In certain embodiments R¹ or R⁴ is

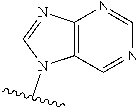 [SMILES: *n1cnc2ncncc21]

optionally substituted with 1, 2, or 3 R⁷ substituents.

In certain embodiments R¹ or R⁴ is

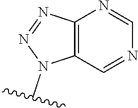 [SMILES: *n1nnc2ncncc21]

optionally substituted with 1 or 2 R⁷ substituents.

In certain embodiments R¹ or R⁴ is

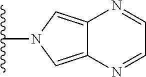 [SMILES: *n1cc2nccnc2c1]

optionally substituted with 1, 2, 3, or 4 R⁷ substituents.

In certain embodiments R¹ or R⁴ is

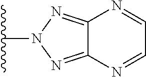 [SMILES: *n1nc2nccnc2n1]

optionally substituted with 1 or 2 R⁷ substituents.

In certain embodiments R¹ or R⁴ is

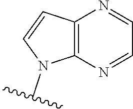 [SMILES: *n1ccc2nccnc21]

optionally substituted with 1, 2, 3, or 4 R⁷ substituents.

In certain embodiments R¹ or R⁴ is

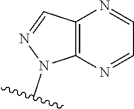 [SMILES: *n1ncc2nccnc21]

optionally substituted with 1, 2, or 3 R⁷ substituents.

In certain embodiments R¹ or R⁴ is

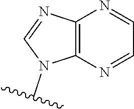 [SMILES: *n1cnc2nccnc21]

optionally substituted with 1, 2, or 3 R⁷ substituents.

In certain embodiments R¹ or R⁴ is

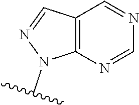 [SMILES: *n1ncc2cncnc21]

optionally substituted with 1, 2, or 3 R⁷ substituents.

In certain embodiments R¹ or R⁴ is

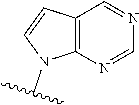 [SMILES: *n1ccc2cncnc21]

optionally substituted with 1, 2, 3, or 4 R⁷ substituents.

In certain embodiments R¹ or R⁴ is

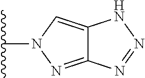 [SMILES: *n1cc2[nH]nnc2n1]

optionally substituted with 1 or 2 R⁷ substituents.

In certain embodiments R¹ or R⁴ is

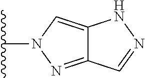 [SMILES: *n1cc2[nH]ncc2n1]

optionally substituted with 1, 2, or 3 R⁷ substituents.

In certain embodiments R¹ or R⁴ is

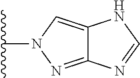 [SMILES: *n1cc2[nH]cnc2n1]

optionally substituted with 1, 2, or 3 R⁷ substituents.

In certain embodiments R¹ or R⁴ is

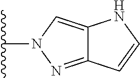 [SMILES: *n1cc2[nH]ccc2n1]

optionally substituted with 1, 2, 3, or 4 R⁷ substituents.

In certain embodiments R¹ or R⁴ is

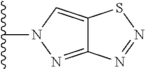 [SMILES: *n1cc2snnc2n1]

optionally substituted with 1 R⁷ substituents.

In certain embodiments R¹ or R⁴ is

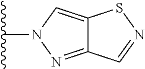 [SMILES: *n1cc2sncc2n1]

optionally substituted with 1 or 2 R⁷ substituents.

In certain embodiments R¹ or R⁴ is

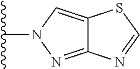 [SMILES: *n1cc2scnc2n1]

optionally substituted with 1 or 2 R⁷ substituents.

In certain embodiments R¹ or R⁴ is

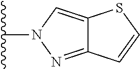 [SMILES: *n1cc2sccc2n1]

optionally substituted with 1, 2, or 3 R⁷ substituents.

In certain embodiments R¹ or R⁴ is

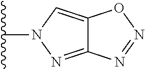 [SMILES: *n1cc2onnc2n1]

optionally substituted with 1 R⁷ substituents.

In certain embodiments R¹ or R⁴ is

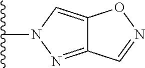 [SMILES: *n1cc2oncc2n1]

optionally substituted with 1 or 2 R⁷ substituents.

In certain embodiments R¹ or R⁴ is

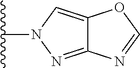 [SMILES: *n1cc2ocnc2n1]

optionally substituted with 1 or 2 R⁷ substituents.

In certain embodiments R¹ or R⁴ is

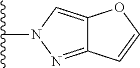 [SMILES: *n1cc2occc2n1]

optionally substituted with 1, 2, or 3 R⁷ substituents.

In certain embodiments R¹ or R⁴ is

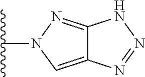 [SMILES: *n1cc2nn[nH]c2n1]

optionally substituted with 1 or 2 R⁷ substituents.

In certain embodiments R¹ or R⁴ is

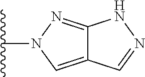 [SMILES: *n1cc2cn[nH]c2n1]

optionally substituted with 1, 2, or 3 R⁷ substituents.

In certain embodiments R¹ or R⁴ is

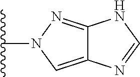 [SMILES: *n1cc2nc[nH]c2n1]

optionally substituted with 1, 2, or 3 R⁷ substituents.

In certain embodiments R¹ or R⁴ is

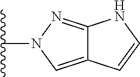 [SMILES: *n1cc2cc[nH]c2n1]

optionally substituted with 1, 2, 3, or 4 R⁷ substituents.

In certain embodiments R¹ or R⁴ is

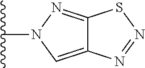 [SMILES: *n1cc2nnsc2n1]

optionally substituted with 1 R⁷ substituents.

In certain embodiments R¹ or R⁴ is

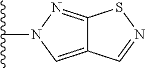 [SMILES: *n1cc2cnsc2n1]

optionally substituted with 1 or 2 R⁷ substituents.

In certain embodiments R¹ or R⁴ is

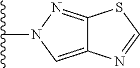 [SMILES: *n1cc2ncsc2n1]

optionally substituted with 1 or 2 R⁷ substituents.

In certain embodiments R¹ or R⁴ is

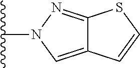 [SMILES: *n1cc2ccsc2n1]

optionally substituted with 1, 2, or 3 R⁷ substituents.

In certain embodiments R¹ or R⁴ is

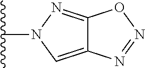 [SMILES: *n1cc2nnoc2n1]

optionally substituted with 1 R⁷ substituents.

In certain embodiments R¹ or R⁴ is

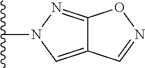 [SMILES: *n1cc2cnoc2n1]

optionally substituted with 1 or 2 R⁷ substituents.

In certain embodiments R¹ or R⁴ is

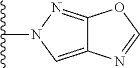 [SMILES: *n1cc2ncoc2n1]

optionally substituted with 1 or 2 R⁷ substituents.

In certain embodiments R¹ or R⁴ is

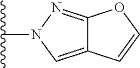 [SMILES: *n1cc2ccoc2n1]

optionally substituted with 1, 2, or 3 R⁷ substituents.

### Embodiments of R² and R³

Bivalent substituents described herein can be either attached in a left to right fashion or a right to left fashion except as excluded by context. For example, where R² is -aryl-C(O)—NR⁶— either the aryl or nitrogen side is attached to the sulfonyl group. For example, when R² is -aryl-C(O)—NR⁶—, Formula I can be

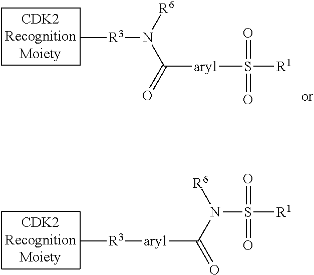

similarly, when —R³— is

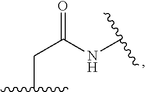 [SMILES: *CC(=O)N*]

### Formula I can be

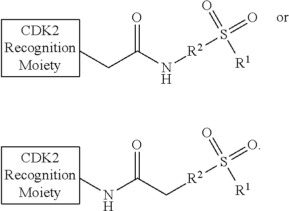

In certain embodiments —R²—R³— and —R³—R²— are selected from:

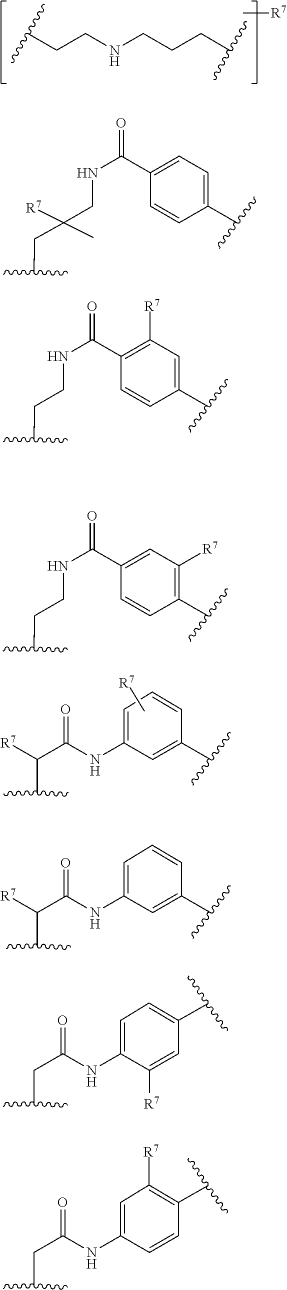

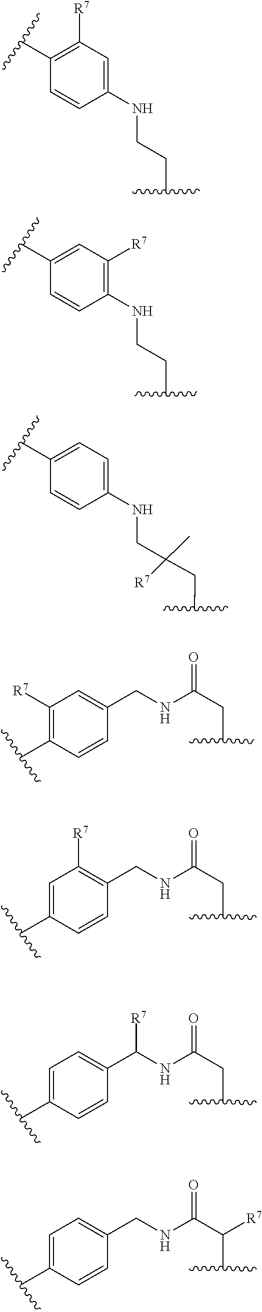

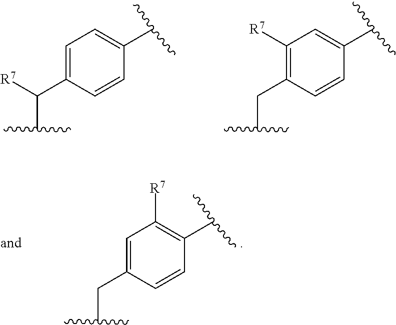

In certain embodiments —R²—R³— and —R³—R²— are selected from:

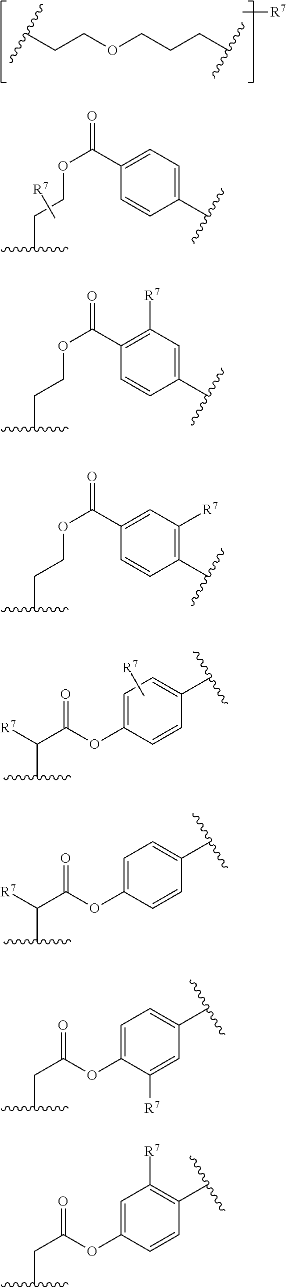

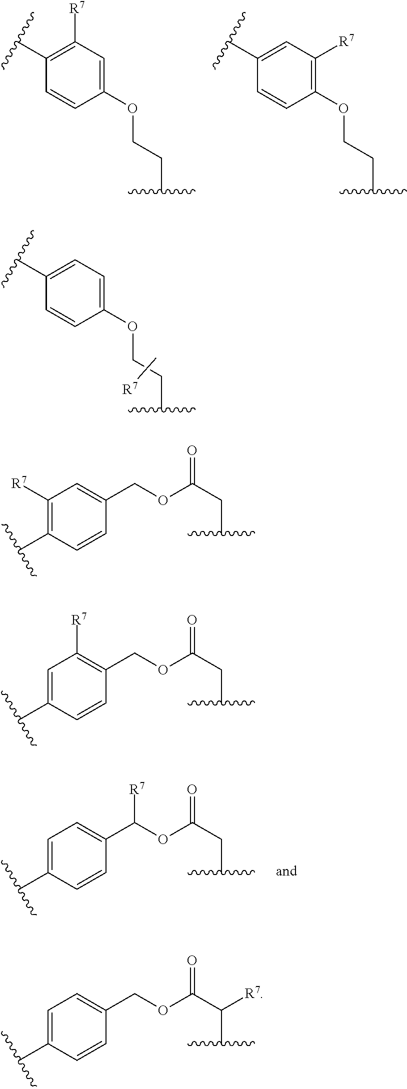

In certain embodiments —R²—R³— and —R³—R²— are selected from:

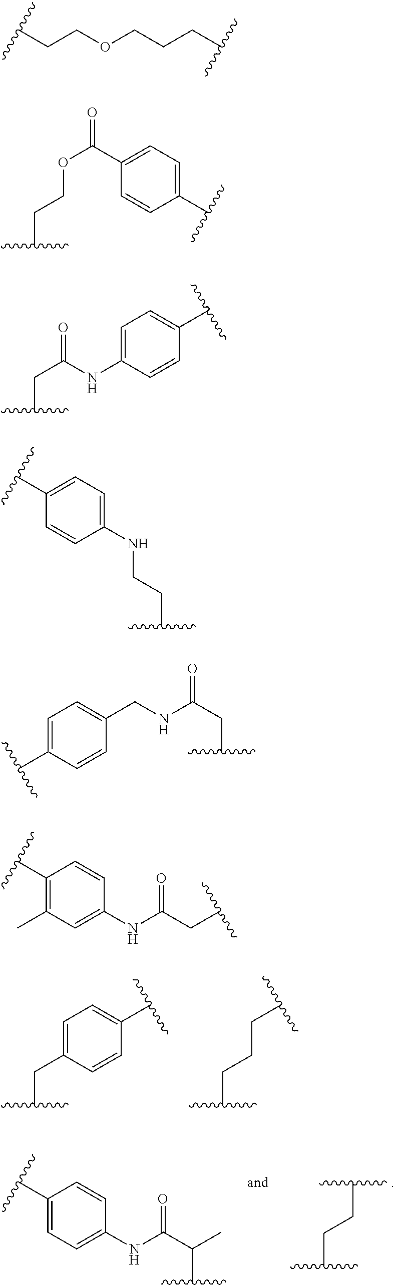

In certain embodiments R² is selected from:

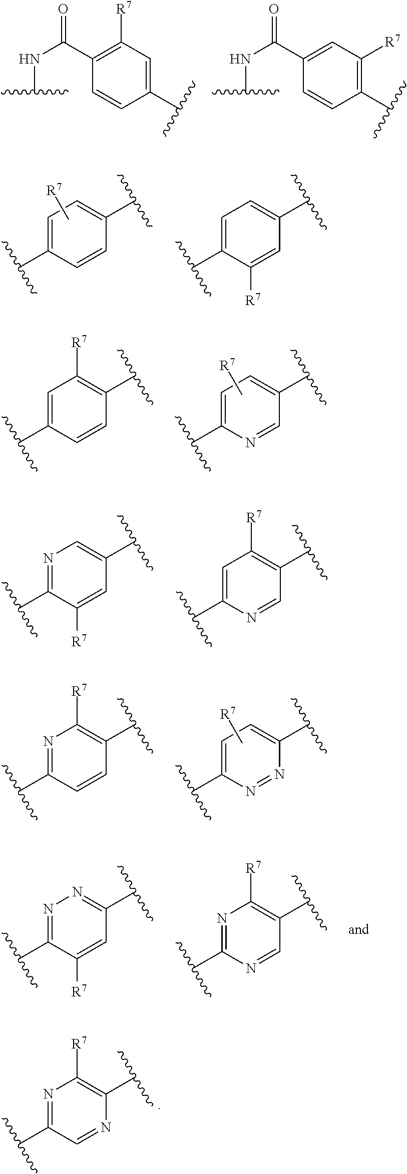

In certain embodiments R² is selected from

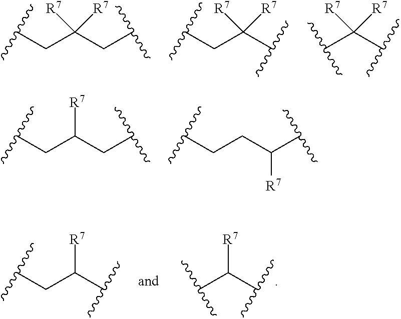

In certain embodiments R² is selected from:

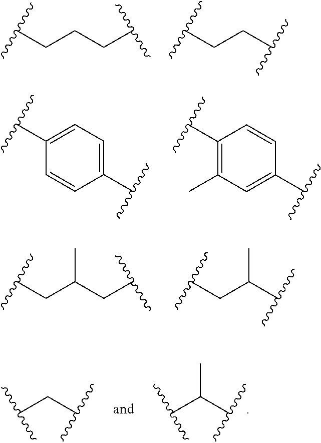

In certain embodiments R² is selected from:

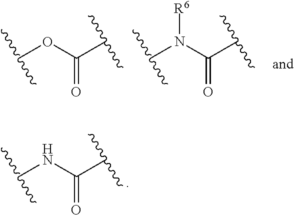

In certain embodiments R³ is selected from

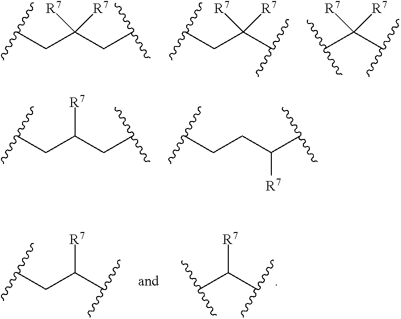

In certain embodiments R³ is selected from:

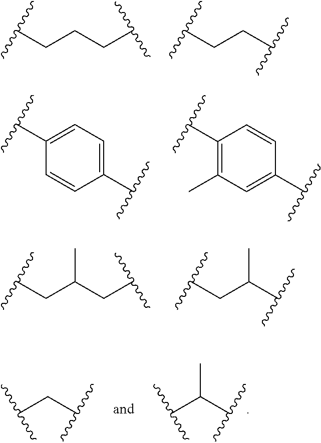

In certain embodiments R³ is selected from:

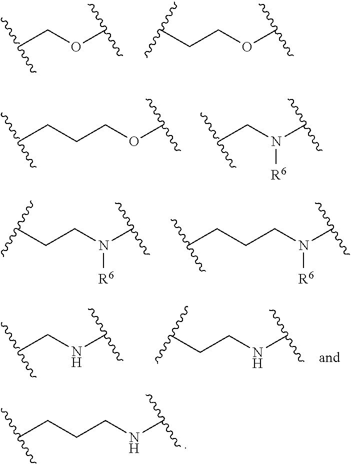

In certain embodiments R³ is selected from:

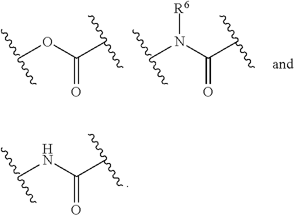

In certain embodiments R³ is selected from:

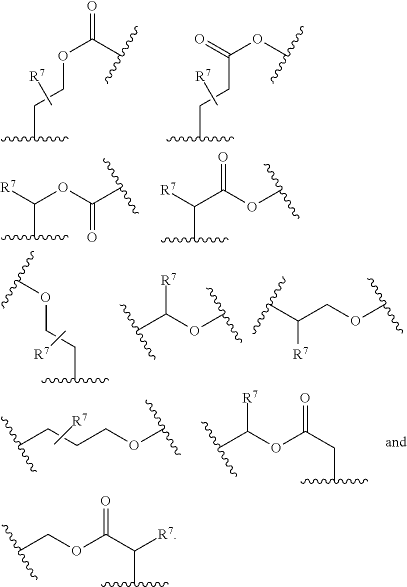

In certain embodiments R³ is selected from:

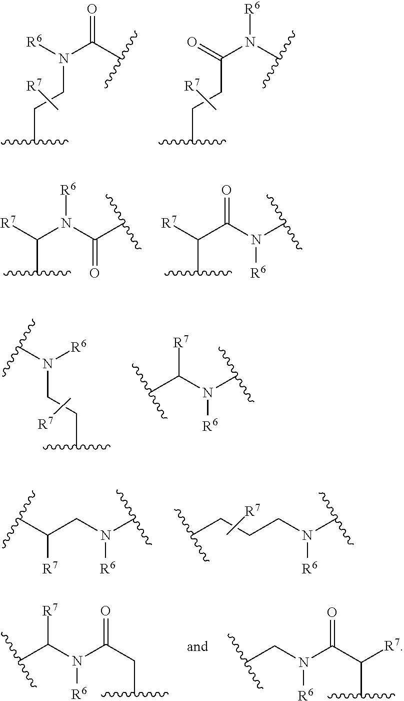

In certain embodiments R³ is selected from:

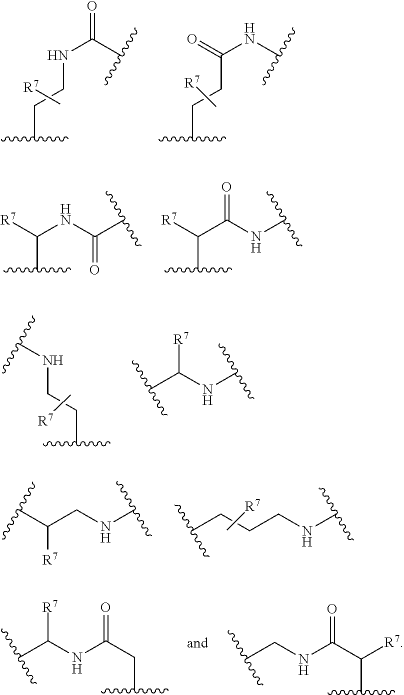

In certain embodiments R² is bond.

In certain embodiments R³ is bond.

In certain embodiments R² and R³ are both bond.

In certain embodiments one of R² and R³ is bond and the other is selected from alkyl, alkenyl, haloalkyl, cycloalkyl, aryl, heterocycle, —S—, —O—, —NR⁶—, —(CH₂)ₚ—C(O)—, —(CH₂)ₚ—C(O)—NR⁶—, —(CH₂CH₂O)ₚ—, —(OCH₂CH₂)ₚ—, —C(O)—, —NR⁶C(O)—, —NR⁶C(O)NR⁶—, —C(O)NR⁶—, —OC(O)NR⁶—, —NR⁶S(O)₂NR⁶—, —S(O)₂NR⁶—, heteroaryl, aryl-C(O)—NR⁶—, heteroaryl-C(O)—NR⁶—, and heteroaryl, each of which is optionally substituted as allowed by valence with 1, 2, 3, or 4 substituents selected from R⁷.

In certain embodiments p is 1.

In certain embodiments p is 2.

In certain embodiments p is 3.

In certain embodiments p is 4.

In certain embodiments p is 5.

In certain embodiments p is 6.

In certain embodiments R² is phenyl.

In certain embodiments R² is phenyl substituted with 1 substituent selected from R⁷.

In certain embodiments R² is phenyl substituted with 2 substituents selected from R⁷.

In certain embodiments R² is phenyl substituted with 3 substituents selected from R⁷.

In certain embodiments R² is phenyl substituted with 4 substituents selected from R⁷.

In certain embodiments R² is phenyl substituted with 1 substituent selected from R^{7EWG}.

In certain embodiments R² is phenyl substituted with 2 substituents selected from R^{7EWG}.

In certain embodiments R² is phenyl substituted with 3 substituents selected from R^{7EWG}.

In certain embodiments R² is phenyl substituted with 4 substituents selected from R^{7EWG}.

R^{7EWG} is independently selected at each instance from halogen, haloalkyl, heterocycle, aryl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, with each haloalkyl, heterocycle, aryl, and heteroaryl optionally substituted as allowed by valence with 1, 2, 3, or 4 substituents selected from R¹⁷.

In certain embodiments R³ is phenyl substituted with 1 substituent selected from R⁷.

In certain embodiments R³ is phenyl substituted with 2 substituents selected from R⁷.

In certain embodiments R³ is phenyl substituted with 3 substituents selected from R⁷.

In certain embodiments R³ is phenyl substituted with 4 substituents selected from R⁷.

In certain embodiments R² is heteroaryl.

In certain embodiments R² is heteroaryl substituted with 1 substituent selected from R⁷.

In certain embodiments R² is heteroaryl substituted with 2 substituents selected from R⁷.

In certain embodiments R² is heteroaryl substituted with 3 substituents selected from R⁷.

In certain embodiments R² is heteroaryl substituted with 4 substituents selected from R⁷.

In certain embodiments R⁴ is

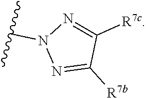

In certain embodiments R⁴ is

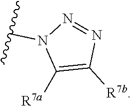

In certain embodiments R⁴ is

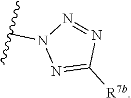

In certain embodiments R⁴ is

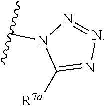

In certain embodiments R⁴ is

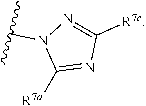

In certain embodiments R⁴ is

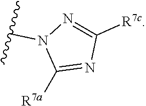

In certain embodiments R⁴ is

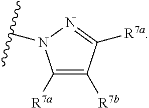

In certain embodiments R⁴ is

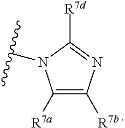

In certain embodiments R⁴ is a 5-membered heteroaryl.

In certain embodiments R⁴ is a fused bicyclic heteroaryl.

In certain embodiments each R⁷ is independently selected from R^{7a}, R^{7b}, R^{7c} and R^{7d}.

In certain embodiments R⁴ is a bicyclic heteroaryl optionally substituted with 1, 2, 3, or 4 R⁷ substituents.

### Embodiments of R⁵

In certain embodiments R⁵ is selected from

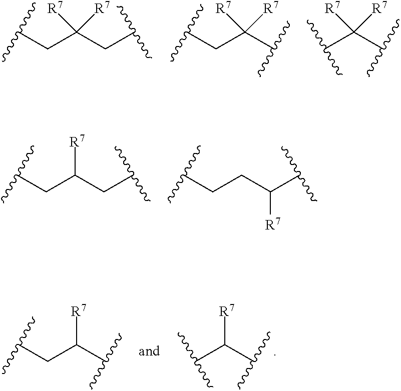

In certain embodiments R⁵ is selected from:

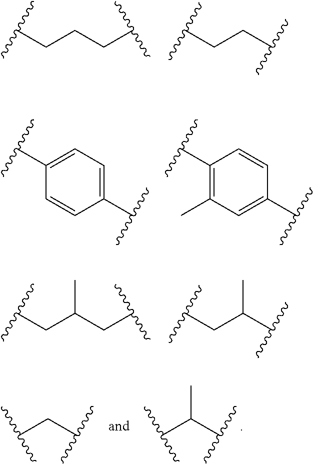

In certain embodiments R⁵ is bond.

In certain embodiments R⁵ and R³ are both bond.

In certain embodiments one of R⁵ and R³ is bond and the other is selected from alkyl, alkenyl, haloalkyl, cycloalkyl, heterocycle, naphthyl, —S—, —O—, —NR⁶—, —(CH₂)ₚ—C(O)—, —(CH₂)ₚ—C(O)—NR⁶—, —(CH₂CH₂O)ₚ—, —(OCH₂CH₂)ₚ—, —C(O)—, —NR⁶C(O)—, —NR⁶C(O)NR⁶—, —C(O)NR⁶—, —OC(O)NR⁶—, —NR⁶S(O)₂NR⁶—, —S(O)₂NR⁶—, heteroaryl, heteroaryl-C(O)—NR⁶—, and heteroaryl, each of which is optionally substituted as allowed by valence with 1, 2, 3, or 4 substituents selected from R⁷.

### Embodiments of R⁶

In certain embodiments one R⁶ is hydrogen.

In certain embodiments one R⁶ is alkyl.

In certain embodiments one R⁶ is haloalkyl.

In certain embodiments one R⁶ is cycloalkyl.

In certain embodiments one R⁶ is aryl.

In certain embodiments one R⁶ is heterocycle.

In certain embodiments one R⁶ is heteroaryl.

### Embodiments of R⁷

In certain embodiments R⁷ is independently selected at each instance from R^{7EWG}.

In certain embodiments R^{7a} is independently selected at each instance from R^{7EWG}.

In certain embodiments R^{7b} is independently selected at each instance from R^{7EWG}.

In certain embodiments R^{7c} is independently selected at each instance from R^{7EWG}.

In certain embodiments R^{7d} is independently selected at each instance from R^{7EWG}.

R^{7EWG} is independently selected at each instance from halogen, haloalkyl, heterocycle, aryl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, with each haloalkyl, heterocycle, aryl, and heteroaryl optionally substituted as allowed by valence with 1, 2, 3, or 4 substituents selected from R¹⁷.

In certain embodiments R⁷ is selected from the group consisting of OH, Oalkyl, NH₂, NHalkyl, Nalkyl₂, haloalkyl, and halogen.

In certain embodiments R⁷ is selected from the group consisting of alkyl, haloalkyl, and halogen.

In certain embodiments one R⁷ is hydrogen.

In certain embodiments one R⁷ is alkyl.

In certain embodiments one R⁷ is cyano.

In certain embodiments one R⁷ is halogen.

In certain embodiments one R⁷ is fluoro.

In certain embodiments one R⁷ is haloalkyl.

In certain embodiments one R⁷ is —CF₃.

In certain embodiments one R⁷ is aryl optionally substituted as allowed by valence with 1, 2, 3, or 4 substituents selected from R¹⁷.

In certain embodiments one R⁷ is phenyl optionally substituted as allowed by valence with 1, 2, 3, or 4 substituents selected from R¹⁷.

In certain embodiments one R⁷ is methyl.

In certain embodiments one R⁷ is CH₂OH.

In certain embodiments R^{7a} is aryl.

In certain embodiments R^{7a} is phenyl.

In certain embodiments R^{7a} is hydrogen.

In certain embodiments R^{7a} is cyano.

In certain embodiments R^{7a} is halogen.

In certain embodiments R^{7a} is fluoro.

In certain embodiments R^{7a} is haloalkyl.

In certain embodiments R^{7a} is —CF₃.

In certain embodiments R^{7a} is aryl optionally substituted as allowed by valence with 1, 2, 3, or 4 substituents selected from R¹⁷.

In certain embodiments R^{7a} is phenyl optionally substituted as allowed by valence with 1, 2, 3, or 4 substituents selected from R¹⁷.

In certain embodiments R^{7a} is aryl.

In certain embodiments R^{7a} is phenyl.

In certain embodiments R^{7b} is hydrogen.

In certain embodiments R^{7b} is cyano.

In certain embodiments R^{7b} is halogen.

In certain embodiments R^{7b} is fluoro.

In certain embodiments R^{7b} is haloalkyl.

In certain embodiments R^{7b} is —CF₃.

In certain embodiments R^{7b} is aryl optionally substituted as allowed by valence with 1, 2, 3, or 4 substituents selected from R¹⁷.

In certain embodiments R^{7b} is phenyl optionally substituted as allowed by valence with 1, 2, 3, or 4 substituents selected from R¹⁷.

In certain embodiments R^{7b} is aryl.

In certain embodiments R^{7b} is phenyl.

In certain embodiments R^{7c} is hydrogen.

In certain embodiments R^{7c} is cyano.

In certain embodiments R^{7c} is halogen.

In certain embodiments R^{7c} is fluoro.

In certain embodiments R^{7c} is haloalkyl.

In certain embodiments R^{7c} is —CF₃.

In certain embodiments R^{7c} is aryl optionally substituted as allowed by valence with 1, 2, 3, or 4 substituents selected from R¹⁷.

In certain embodiments R^{7c} is phenyl optionally substituted as allowed by valence with 1, 2, 3, or 4 substituents selected from R¹⁷.

In certain embodiments R^{7c} is aryl.

In certain embodiments R^{7c} is phenyl.

In certain embodiments R^{7d} is hydrogen.

In certain embodiments R^{7d} is cyano.

In certain embodiments R^{7d} is halogen.

In certain embodiments R^{7d} is fluoro.

In certain embodiments R^{7d} is haloalkyl.

In certain embodiments R^{7d} is —CF₃.

In certain embodiments R^{7d} is aryl optionally substituted as allowed by valence with 1, 2, 3, or 4 substituents selected from R¹⁷.

In certain embodiments R^{7d} is phenyl optionally substituted as allowed by valence with 1, 2, 3, or 4 substituents selected from R¹⁷.

In certain embodiments R^{7d} is aryl.

In certain embodiments R^{7d} is phenyl.

### Embodiments of R^{8a}, R^{8b}, R^{8c}, and R^{8d}

In certain embodiments R^{8a} is R¹².

In certain embodiments R^{8b} is R¹².

In certain embodiments R^{8c} is R¹².

In certain embodiments R^{8d} is R¹².

In certain embodiments R^{8a} is R¹² and R^{8b}, R^{8c}, and R^{8d} are hydrogen.

In certain embodiments R^{8b} is R¹² and R^{8c}, R^{8d}, and R^{8a} are hydrogen.

In certain embodiments R^{8c} is R¹² and R^{8b}, R^{8d}, and R^{8a} are hydrogen.

In certain embodiments R^{8d} is R¹² and R^{8b}, R^{8c}, and R^{8a} are hydrogen.

In certain embodiments R^{8a} is cyano.

In certain embodiments R^{8a} is halogen.

In certain embodiments R^{8a} is fluoro.

In certain embodiments R^{8a} is haloalkyl.

In certain embodiments R^{8a} is —CF₃.

In certain embodiments R^{8a} is aryl optionally substituted as allowed by valence with 1, 2, 3, or 4 substituents selected from R¹⁷.

In certain embodiments R^{8a} is phenyl optionally substituted as allowed by valence with 1, 2, 3, or 4 substituents selected from R¹⁷.

In certain embodiments R^{8a} is aryl.

In certain embodiments R^{8a} is phenyl.

In certain embodiments R^{8a} is OR⁶.

In certain embodiments R^{8a} is N(R⁶)₂.

In certain embodiments R^{8b} is cyano.

In certain embodiments R^{8b} is halogen.

In certain embodiments R^{8b} is fluoro.

In certain embodiments R^{8b} is haloalkyl.

In certain embodiments R^{8b} is —CF₃.

In certain embodiments R^{8b} is aryl optionally substituted as allowed by valence with 1, 2, 3, or 4 substituents selected from R¹⁷.

In certain embodiments R^{8b} is phenyl optionally substituted as allowed by valence with 1, 2, 3, or 4 substituents selected from R¹⁷.

In certain embodiments R^{8b} is aryl.

In certain embodiments R^{8b} is phenyl.

In certain embodiments R^{8b} is OR⁶.

In certain embodiments R^{8b} is N(R⁶)₂.

In certain embodiments R^{8c} is cyano.

In certain embodiments R^{8c} is halogen.

In certain embodiments R^{8c} is fluoro.

In certain embodiments R^{8c} is haloalkyl.

In certain embodiments R^{8c} is —CF₃.

In certain embodiments R^{8c} is aryl optionally substituted as allowed by valence with 1, 2, 3, or 4 substituents selected from R¹⁷.

In certain embodiments R^{8c} is phenyl optionally substituted as allowed by valence with 1, 2, 3, or 4 substituents selected from R¹⁷.

In certain embodiments R^{8c} is aryl.

In certain embodiments R^{8c} is phenyl.

In certain embodiments R^{8c} is OR⁶.

In certain embodiments R^{8d} is N(R⁶)₂.

In certain embodiments R^{8d} is cyano.

In certain embodiments R^{8d} is halogen.

In certain embodiments R^{8d} is fluoro.

In certain embodiments R^{8d} is haloalkyl.

In certain embodiments R^{8d} is —CF₃.

In certain embodiments R^{8d} is aryl optionally substituted as allowed by valence with 1, 2, 3, or 4 substituents selected from R¹⁷.

In certain embodiments R^{8d} is phenyl optionally substituted as allowed by valence with 1, 2, 3, or 4 substituents selected from R¹⁷.

In certain embodiments R^{8d} is aryl.

In certain embodiments R^{8d} is phenyl.

In certain embodiments R^{8d} is OR⁶.

In certain embodiments R^{8d} is N(R⁶)₂.

### Embodiments of R⁹

In certain embodiments —R²—R⁹— and —R⁹—R²— are selected from:

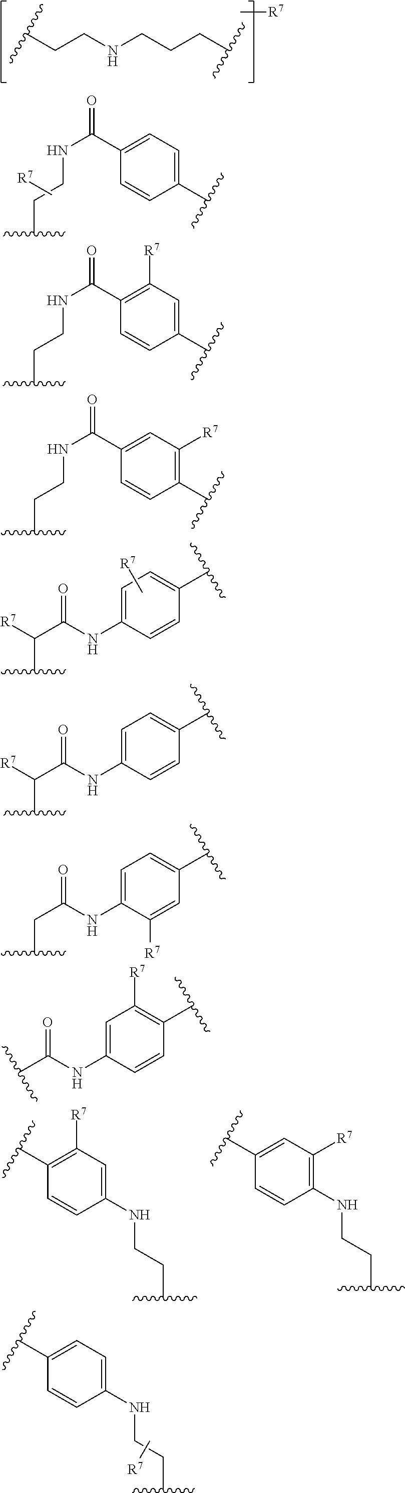

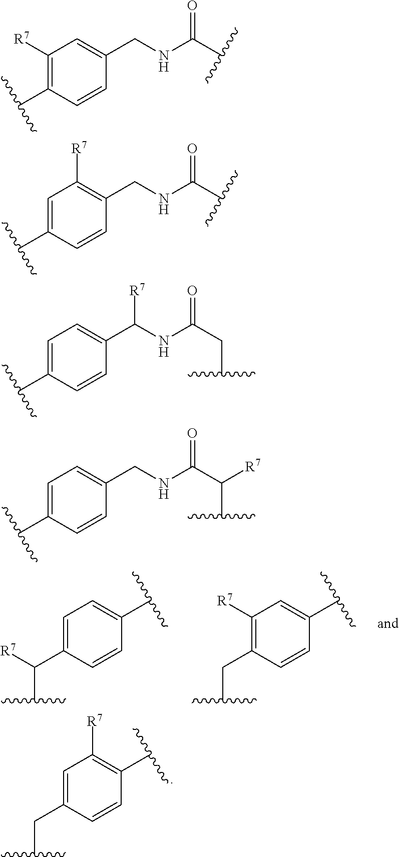

In certain embodiments —R²—R⁹— and —R⁹—R²— are selected from:

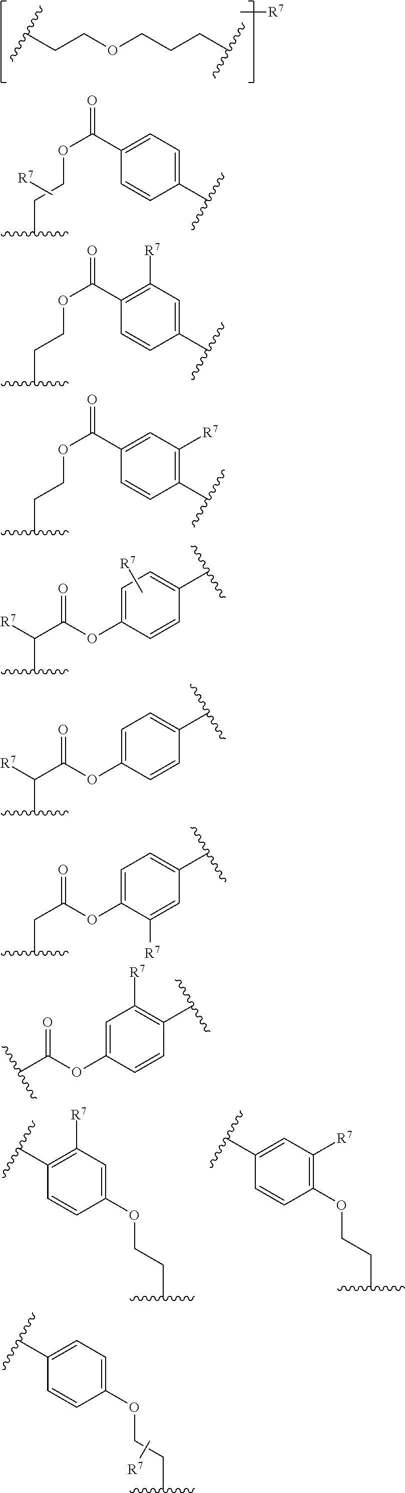

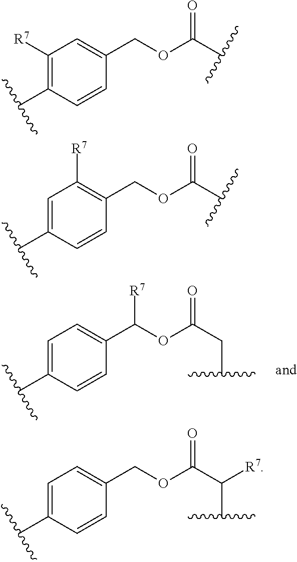

In certain embodiments —R⁹—R³— and —R⁹—R²— are selected from:

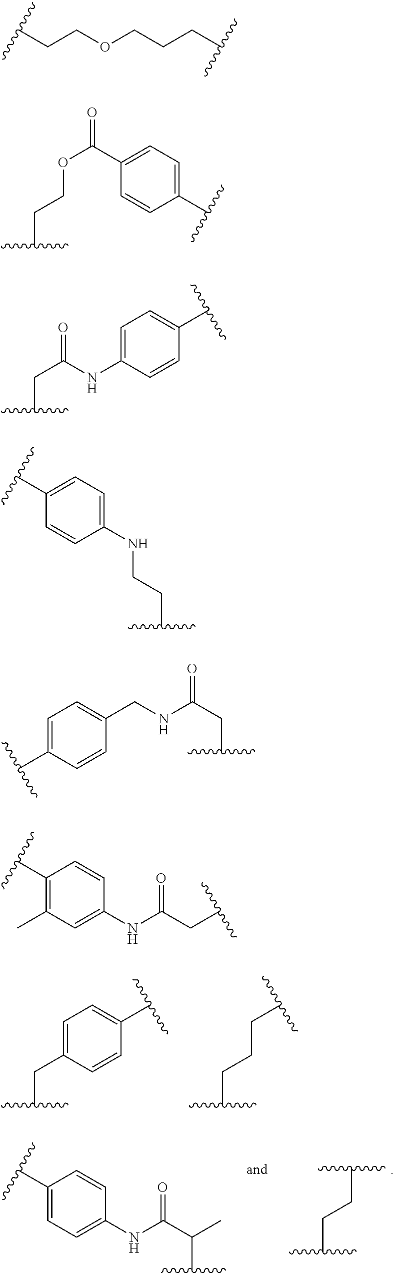

In certain embodiments R⁹ is selected from

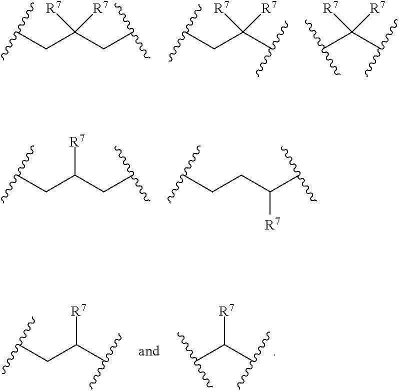

In certain embodiments R⁹ is selected from

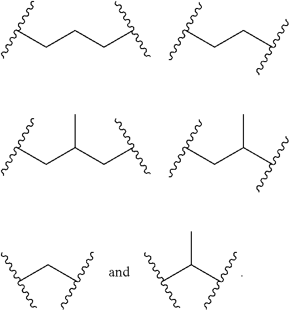

In certain embodiments R⁹ is selected from:

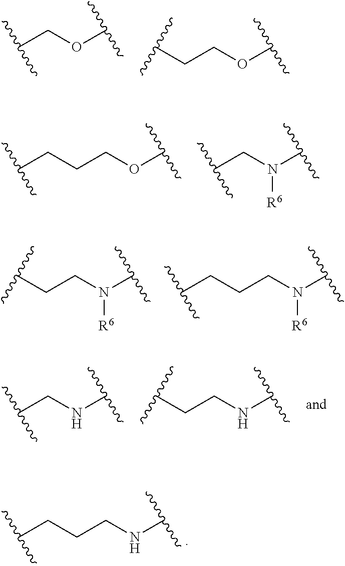

In certain embodiments R⁹ is selected from:

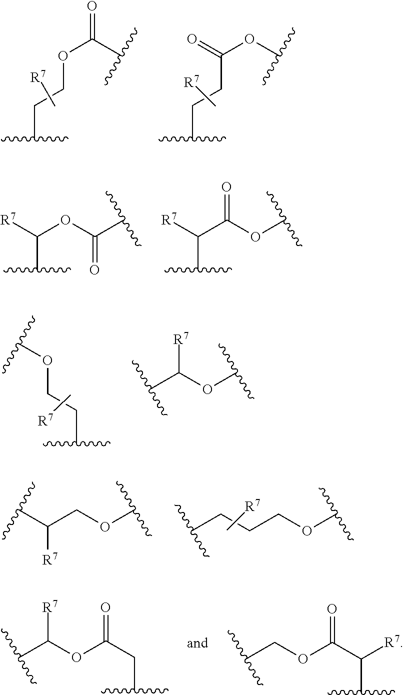

In certain embodiments R⁹ is selected from:

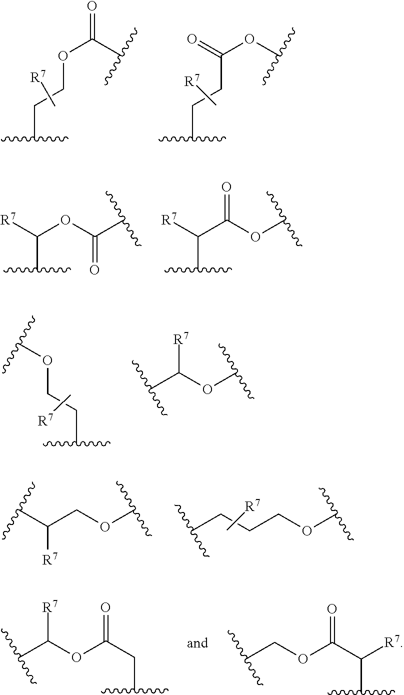

In certain embodiments R² is bond and R⁹ is selected from alkyl, alkenyl, haloalkyl, cycloalkyl, heterocycle, —NR⁶C(O)—, —NR⁶C(O)NR⁶—, —C(O)NR⁶—, —OC(O)NR⁶—, —NR⁶S(O)₂NR⁶—, —S(O)₂NR⁶—, and heteroaryl, each of which is optionally substituted as allowed by valence with 1, 2, 3, or 4 substituents selected from R⁷.

### Embodiments of R¹¹

In certain embodiments R¹¹ is hydrogen.

In certain embodiments R¹¹ is cyano.

In certain embodiments R¹¹ is halogen.

In certain embodiments R¹¹ is fluoro.

In certain embodiments R¹¹ is haloalkyl.

In certain embodiments R¹¹ is —CF₃.

In certain embodiments R¹¹ is naphthyl optionally substituted as allowed by valence with 1, 2, 3, or 4 substituents selected from R¹⁷.

In certain embodiments R¹¹ is naphthyl.

### Embodiments of R¹²

In certain embodiments R¹² is cyano.

In certain embodiments R¹² is halogen.

In certain embodiments R¹² is fluoro.

In certain embodiments R¹² is haloalkyl.

In certain embodiments R¹² is —CF₃.

In certain embodiments R¹² is aryl optionally substituted as allowed by valence with 1, 2, 3, or 4 substituents selected from R⁷.

In certain embodiments R¹² is aryl.

In certain embodiments R¹² is phenyl optionally substituted as allowed by valence with 1, 2, 3, or 4 substituents selected from R⁷.

In certain embodiments R¹² is phenyl.

### Embodiments of R¹³

In certain embodiments R¹³ is cycloalkyl.

In certain embodiments R¹³ is cyclopropyl.

In certain embodiments R¹³ is aryl optionally substituted as allowed by valence with 1, 2, 3, or 4 substituents selected from R⁷.

In certain embodiments R¹³ is phenyl optionally substituted as allowed by valence with 1, 2, 3, or 4 substituents selected from R⁷.

In certain embodiments R¹³ is heteroaryl optionally substituted as allowed by valence with 1, 2, 3, or 4 substituents selected from R⁷.

In certain embodiments R¹³ is aryl.

In certain embodiments R¹³ is phenyl.

In certain embodiments R¹³ is heteroaryl.

In certain embodiments R¹³ is haloalkyl.

### Embodiments of R¹⁵

In certain embodiments R¹⁵ is selected from

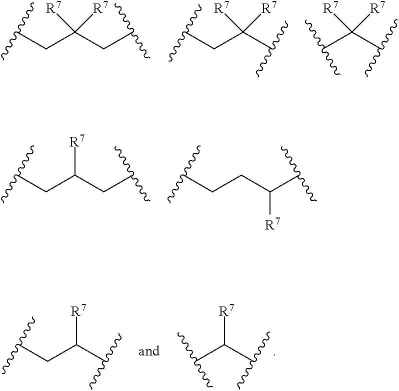

In certain embodiments R¹⁵ is selected from:

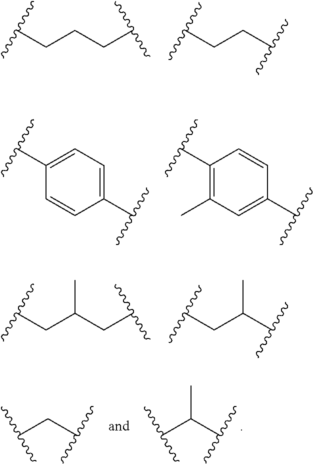

In certain embodiments R¹⁵ is selected from:

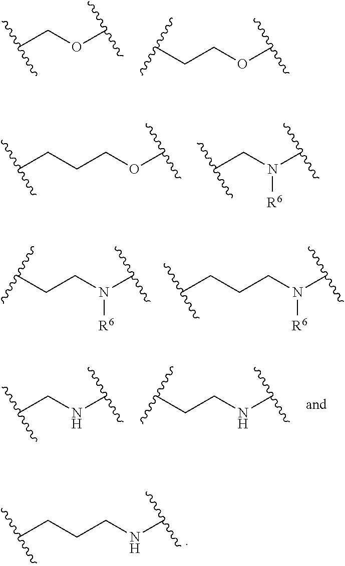

In certain embodiments R¹⁵ is selected from:

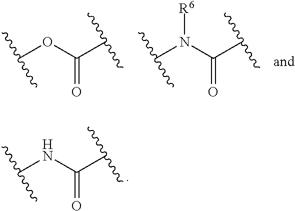

In certain embodiments R¹⁵ is selected from:

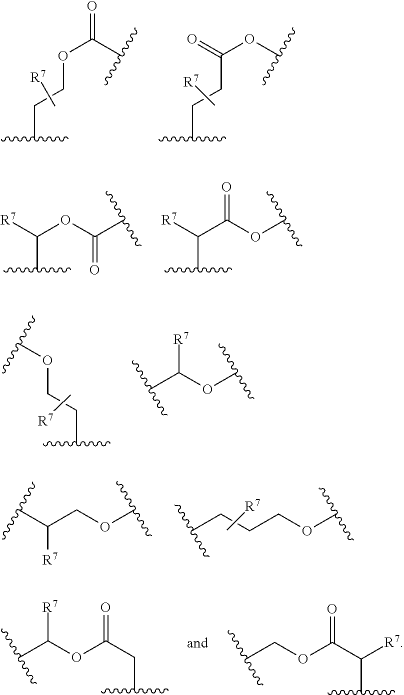

In certain embodiments R¹⁵ is selected from:

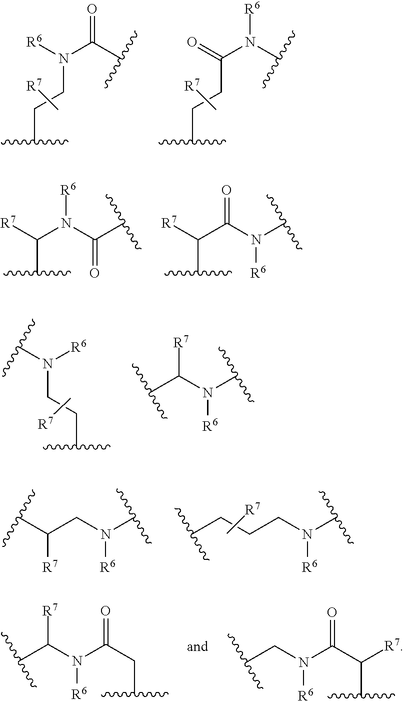

In certain embodiments R¹⁵ is selected from:

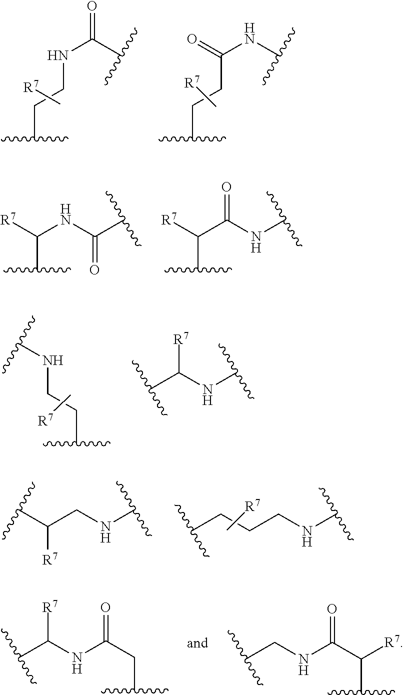

In certain embodiments R¹⁵ is phenyl.

In certain embodiments R¹⁵ is phenyl substituted with 1 substituent selected from R⁷.

In certain embodiments R¹⁵ is phenyl substituted with 2 substituents selected from R⁷.

In certain embodiments R¹⁵ is phenyl substituted with 3 substituents selected from R⁷.

In certain embodiments R¹⁵ is phenyl substituted with 4 substituents selected from R⁷.

In certain embodiments R¹⁵ is heteroaryl.

In certain embodiments R¹⁵ is heteroaryl substituted with 1 substituent selected from R⁷.

In certain embodiments R¹⁵ is heteroaryl substituted with 2 substituents selected from R⁷.

In certain embodiments R¹⁵ is heteroaryl substituted with 3 substituents selected from R⁷.

In certain embodiments R¹⁵ is heteroaryl substituted with 4 substituents selected from R⁷.

### Embodiments of R¹⁶

In certain embodiments R¹⁶ is

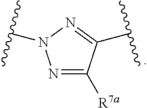

In certain embodiments R¹⁶ is

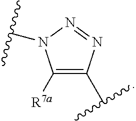

In certain embodiments R¹⁶ is

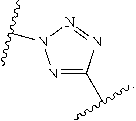 [SMILES: *c1nnn(*)n1]

In certain embodiments R¹⁶ is

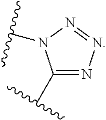 [SMILES: *c1nnnn1*]

In certain embodiments R¹⁶ is

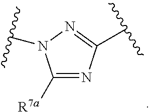

In certain embodiments R¹⁶ is

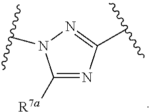

In certain embodiments R¹⁶ is

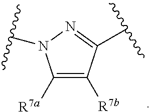

In certain embodiments R¹⁶ is

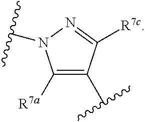

In certain embodiments R¹⁶ is

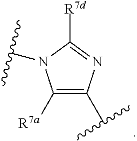

In certain embodiments R¹⁶ is a fused bicyclic heteroaryl.

### Embodiments of R¹⁷

In certain embodiments R¹⁷ is hydrogen.

In certain embodiments R¹⁷ is cyano.

In certain embodiments R¹⁷ is halogen.

In certain embodiments R¹⁷ is fluoro.

In certain embodiments R¹⁷ is haloalkyl.

In certain embodiments R¹⁷ is —CF₃.

In certain embodiments R¹⁷ is aryl.

In certain embodiments R¹⁷ is phenyl.

In certain embodiments one R¹⁷ is hydrogen.

In certain embodiments one R¹⁷ is cyano.

In certain embodiments one R¹⁷ is halogen.

In certain embodiments one R¹⁷ is fluoro.

In certain embodiments one R¹⁷ is haloalkyl.

In certain embodiments one R¹⁷ is —CF₃.

In certain embodiments one R¹⁷ is aryl.

In certain embodiments one R¹⁷ is phenyl.

### Embodiments of R¹⁸

In certain embodiments R¹⁸ is hydrogen.

In certain embodiments R¹⁸ is halogen.

In certain embodiments R¹⁸ is alkyl.

In certain embodiments R¹⁸ is haloalkyl.

In certain embodiments R¹⁸ is alkenyl.

In certain embodiments R¹⁸ is cycloalkyl.

In certain embodiments R¹⁸ is heterocycle.

In certain embodiments R¹⁸ is aryl.

In certain embodiments R¹⁸ is heteroaryl.

In certain embodiments R¹⁸ is cyano.

In certain embodiments R¹⁸ is nitro.

In certain embodiments one R¹⁸ is hydrogen.

In certain embodiments one R¹⁸ is halogen.

In certain embodiments one R¹⁸ is alkyl.

In certain embodiments one R¹⁸ is haloalkyl.

In certain embodiments one R¹⁸ is alkenyl.

In certain embodiments one R¹⁸ is cycloalkyl.

In certain embodiments one R¹⁸ is heterocycle.

In certain embodiments one R¹⁸ is aryl.

In certain embodiments one R¹⁸ is heteroaryl.

In certain embodiments one R¹⁸ is cyano.

In certain embodiments one R¹⁸ is nitro.

### Embodiments of R¹⁹

In certain embodiments R¹⁹ is hydrogen.

In certain embodiments R¹⁹ is alkyl.

In certain embodiments R¹⁹ is haloalkyl.

In certain embodiments R¹⁹ is cycloalkyl.

In certain embodiments R¹⁹ is heterocycle.

In certain embodiments R¹⁹ is aryl.

In certain embodiments R¹⁹ is heteroaryl.

In certain embodiments one R¹⁹ is hydrogen.

In certain embodiments one R¹⁹ is alkyl.

In certain embodiments one R¹⁹ is haloalkyl.

In certain embodiments one R¹⁹ is cycloalkyl.

In certain embodiments one R¹⁹ is heterocycle.

In certain embodiments one R¹⁹ is aryl.

In certain embodiments one R¹⁹ is heteroaryl.

### Embodiments of R²⁷

In certain embodiments R²⁷ is hydrogen.

In certain embodiments R²⁷ is cyano.

In certain embodiments R²⁷ is halogen.

In certain embodiments R²⁷ is fluoro.

In certain embodiments R²⁷ is haloalkyl.

In certain embodiments R²⁷ is —CF₃.

In certain embodiments R²⁷ is aryl.

In certain embodiments R²⁷ is phenyl.

In certain embodiments one R²⁷ is hydrogen.

In certain embodiments one R²⁷ is cyano.

In certain embodiments one R²⁷ is halogen.

In certain embodiments one R²⁷ is fluoro.

In certain embodiments one R²⁷ is haloalkyl.

In certain embodiments one R²⁷ is —CF₃.

In certain embodiments one R²⁷ is aryl.

In certain embodiments one R²⁷ is phenyl.

In certain embodiments one R²⁷ is selected from:

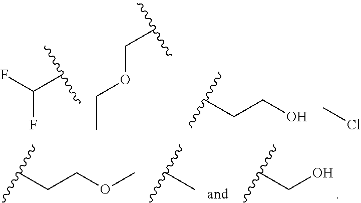

In certain embodiments one R²⁷ is haloalkyl.

In certain embodiments one R²⁷ is -alkyl-hydroxy.

In certain embodiments one R²⁷ is -alkyl-hydroxy-alkyl.

In certain embodiments one R²⁷ is halogen.

In certain embodiments at least one R²⁷ is halogen.

In certain embodiments one R²⁷ is alkyl.

In certain embodiments one R²⁷ is methyl.

In certain embodiments at least one R²⁷ is alkyl.

In certain embodiments at least one R²⁷ is methyl.

### Embodiments of R²⁹

In certain embodiments R²⁹ is a heterocycle.

In certain embodiments R²⁹ is a heterocycle substituted by a 1, 2, or 3 substituents selected from SO₂Me, halogen, and alkyl.

In certain embodiments R²⁹ is a heterocycle substituted with SO₂Me.

In certain embodiments R²⁹ is a heterocycle substituted by a 1, 2, or 3 substituents selected halogen.

In certain embodiments R²⁹ is a heterocycle substituted with alkyl.

In certain embodiments R²⁹ is an -alkyl-cycloalkyl.

In certain embodiments R²⁹ is selected from:

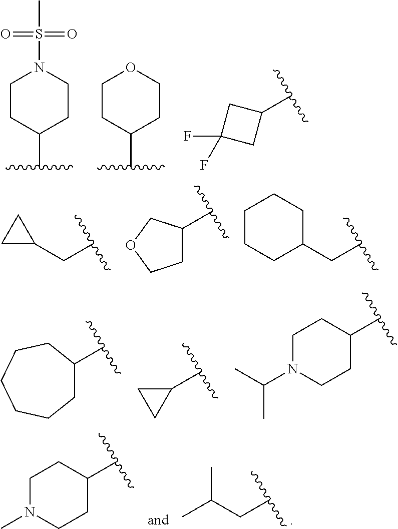

In certain embodiments R²⁹ is:

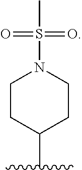 [SMILES: *C1CCN(S(C)(=O)=O)CC1]

### Embodiments of “Alkyl”

In one embodiment “alkyl” is a C₁-C₁₀alkyl, C₁-C₉alkyl, C₁-C₈alkyl, C₁-C₇alkyl, C₁-C₆alkyl, C₁-C₅alkyl, C₁-C₄alkyl, C₁-C₃alkyl, or C₁-C₂alkyl.

In one embodiment “alkyl” has one carbon.

In one embodiment “alkyl” has two carbons.

In one embodiment “alkyl” has three carbons.

In one embodiment “alkyl” has four carbons.

In one embodiment “alkyl” has five carbons.

In one embodiment “alkyl” has six carbons.

Non-limiting examples of “alkyl” include: methyl, ethyl, propyl, butyl, pentyl, and hexyl.

Additional non-limiting examples of “alkyl” include: isopropyl, isobutyl, isopentyl, and isohexyl.

Additional non-limiting examples of “alkyl” include: sec-butyl, sec-pentyl, and sec-hexyl.

Additional non-limiting examples of “alkyl” include: tert-butyl, tert-pentyl, and tert-hexyl.

Additional non-limiting examples of “alkyl” include: neopentyl, 3-pentyl, and active pentyl.

In an alternative embodiment the “alkyl” group is optionally substituted.

In an alternative embodiment the “alkenyl” group is optionally substituted.

### Embodiments of “Haloalkyl”

In one embodiment “haloalkyl” is a C₁-C₁₀haloalkyl, C₁-C₉haloalkyl, C₁-C₈haloalkyl, C₁-C₇haloalkyl, C₁-C₆haloalkyl, C₁-C₅haloalkyl, C₁-C₄haloalkyl, C₁-C₃haloalkyl, and C₁-C₂haloalkyl.

In one embodiment “haloalkyl” has one carbon.

In one embodiment “haloalkyl” has one carbon and one halogen.

In one embodiment “haloalkyl” has one carbon and two halogens.

In one embodiment “haloalkyl” has one carbon and three halogens.

In one embodiment “haloalkyl” has two carbons.

In one embodiment “haloalkyl” has three carbons.

In one embodiment “haloalkyl” has four carbons.

In one embodiment “haloalkyl” has five carbons.

In one embodiment “haloalkyl” has six carbons.

Non-limiting examples of “haloalkyl” include:

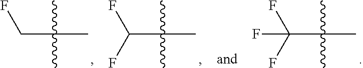

Additional non-limiting examples of “haloalkyl” include:

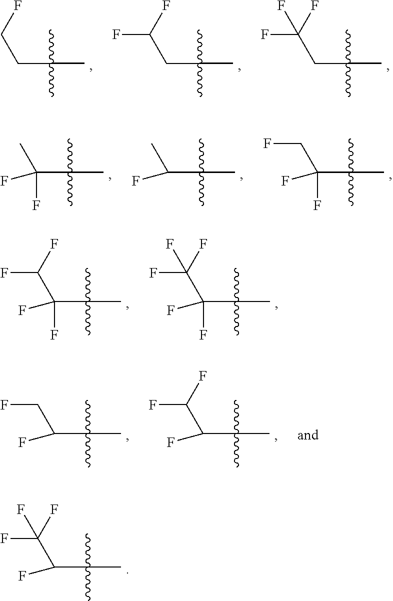

Additional non-limiting examples of “haloalkyl” include:

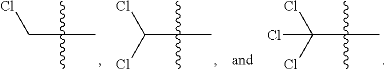

Additional non-limiting examples of “haloalkyl” include:

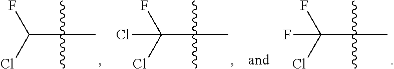

### Embodiments of “Heteroaryl”

Non-limiting examples of 5 membered “heteroaryl” groups include pyrrole, furan, thiophene, pyrazole, imidazole, triazole, isoxazole, oxazole, oxadiazole, oxatriazole, isothiazole, thiazole, thiadiazole, and thiatriazole.

Additional non-limiting examples of 5 membered “heteroaryl” groups include:

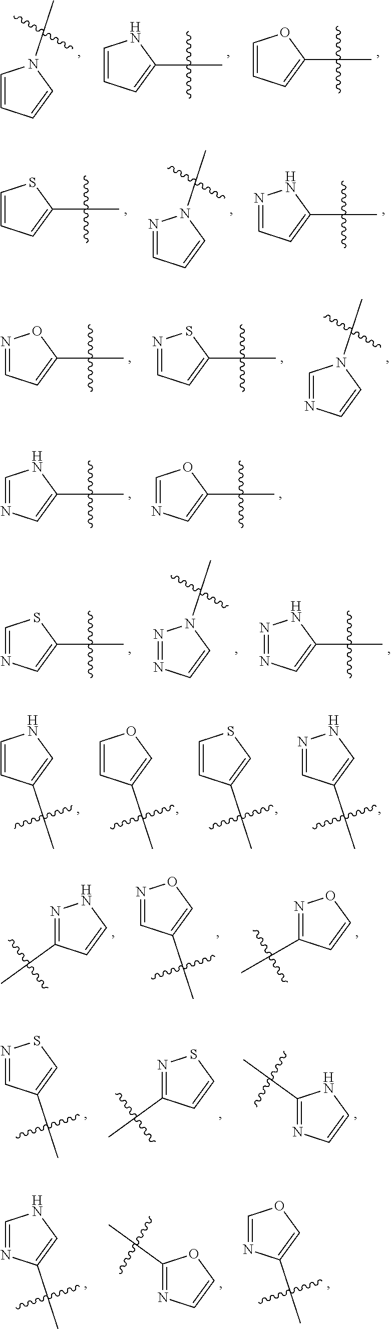

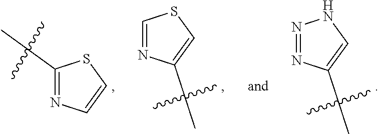 [SMILES: *c1nccs1 ; *c1cscn1 ; *c1c[nH]nn1]

In one embodiment “heteroaryl” is a 6 membered aromatic group containing 1, 2, or 3 nitrogen atoms (i.e. pyridinyl, pyridazinyl, triazinyl, pyrimidinyl, and pyrazinyl).

Non-limiting examples of 6 membered “heteroaryl” groups with 1 or 2 nitrogen atoms include:

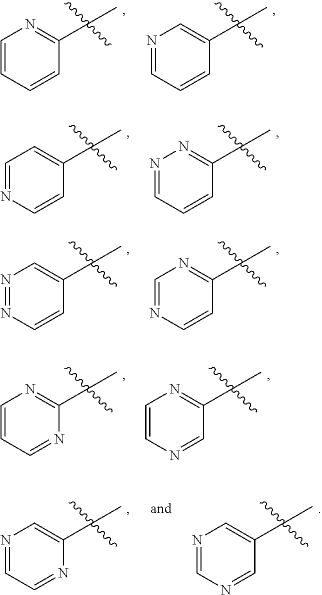

In one embodiment “heteroaryl” is a 9 membered bicyclic aromatic group containing 1 or 2 atoms selected from nitrogen, oxygen, and sulfur.

Non-limiting examples of“heteroaryl” groups that are bicyclic include indole, benzofuran, isoindole, indazole, benzimidazole, azaindole, azaindazole, purine, isobenzofuran, benzothiophene, benzoisoxazole, benzoisothiazole, benzooxazole, and benzothiazole.

Additional non-limiting examples of “heteroaryl” groups that are bicyclic include:

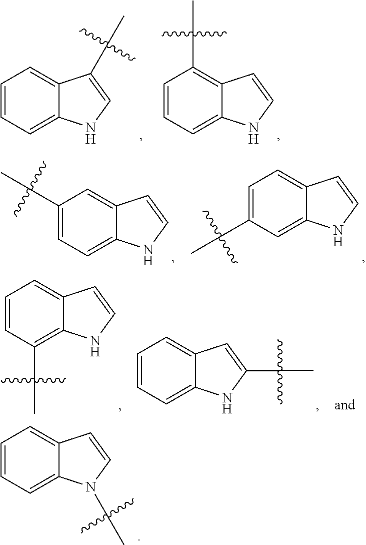

Additional non-limiting examples of “heteroaryl” groups that are bicyclic include:

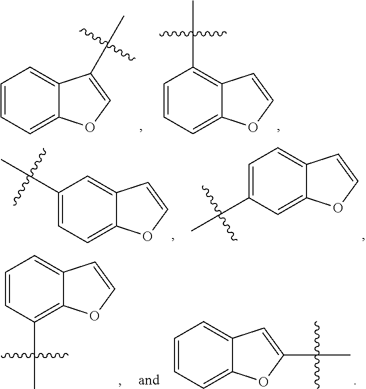

Additional non-limiting examples of “heteroaryl” groups that are bicyclic include:

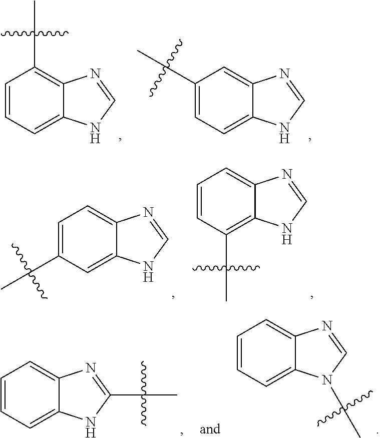

In one embodiment “heteroaryl” is a 10 membered bicyclic aromatic group containing 1 or 2 atoms selected from nitrogen, oxygen, and sulfur.

Non-limiting examples of “heteroaryl” groups that are bicyclic include quinoline, isoquinoline, quinoxaline, phthalazine, quinazoline, cinnoline, and naphthyridine.

Additional non-limiting examples of “heteroaryl” groups that are bicyclic include:

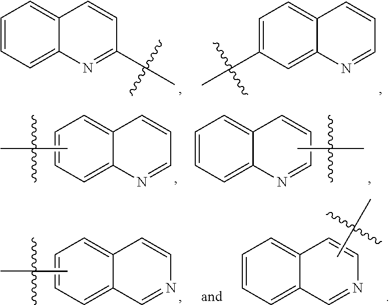

### Embodiments of “Heterocycle”

In one embodiment “heterocycle” refers to a cyclic ring with one nitrogen and 3, 4, 5, 6, 7, or 8 carbon atoms.

In one embodiment “heterocycle” refers to a cyclic ring with one nitrogen and one oxygen and 3, 4, 5, 6, 7, or 8 carbon atoms.

In one embodiment “heterocycle” refers to a cyclic ring with two nitrogens and 3, 4, 5, 6, 7, or 8 carbon atoms.

In one embodiment “heterocycle” refers to a cyclic ring with one oxygen and 3, 4, 5, 6, 7, or 8 carbon atoms.

In one embodiment “heterocycle” refers to a cyclic ring with one sulfur and 3, 4, 5, 6, 7, or 8 carbon atoms.

Non-limiting examples of “heterocycle” include aziridine, oxirane, thiirane, azetidine, 1,3-diazetidine, oxetane, and thietane.

Additional non-limiting examples of “heterocycle” include pyrrolidine, 3-pyrroline, 2-pyrroline, pyrazolidine, and imidazolidine.

Additional non-limiting examples of “heterocycle” include tetrahydrofuran, 1,3-dioxolane, tetrahydrothiophene, 1,2-oxathiolane, and 1,3-oxathiolane.

Additional non-limiting examples of “heterocycle” include piperidine, piperazine, tetrahydropyran, 1,4-dioxane, thiane, 1,3-dithiane, 1,4-dithiane, morpholine, and thiomorpholine.

Additional non-limiting examples of “heterocycle” include indoline, tetrahydroquinoline, tetrahydroisoquinoline, and dihydrobenzofuran wherein the point of attachment for each group is on the heterocyclic ring.

For example,

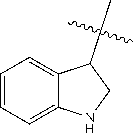 [SMILES: *C1CNc2ccccc21]

is a “heterocycle” group.

However,

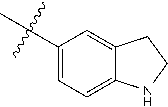 [SMILES: *c1ccc2c(c1)CCN2]

is an “aryl” group.

Non-limiting examples of “heterocycle” also include:

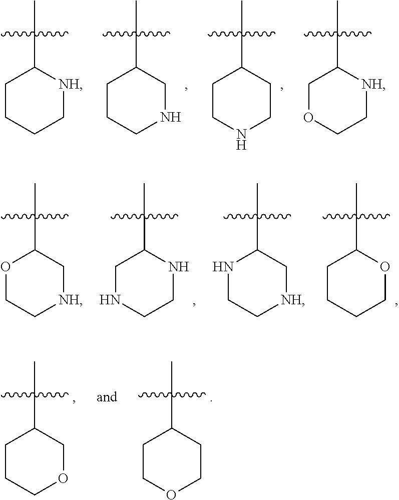

Additional non-limiting examples of “heterocycle” include:

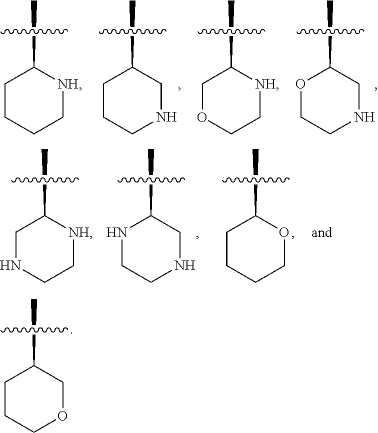

Additional non-limiting examples of “heterocycle” include:

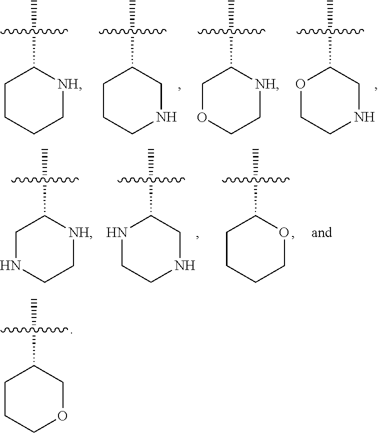

Non-limiting examples of “heterocycle” also include:

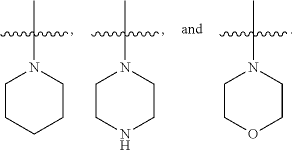

Non-limiting examples of “heterocycle” also include:

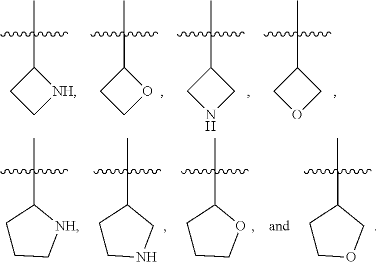

Additional non-limiting examples of “heterocycle” include:

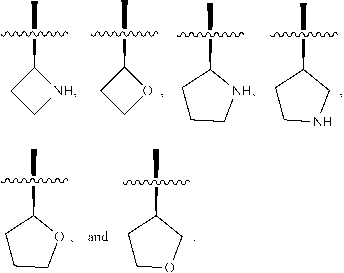

Additional non-limiting examples of “heterocycle” include:

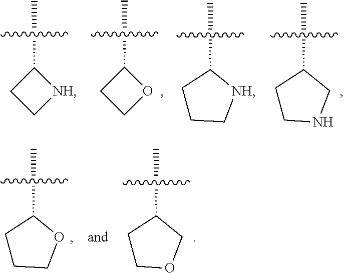

### Embodiments of “Aryl”

In one embodiment “aryl” is a 6 carbon aromatic group (phenyl).

In one embodiment “aryl” is a 10 carbon aromatic group (naphthyl).

In one embodiment “aryl” is a 6 carbon aromatic group fused to a heterocycle wherein the point of attachment is the aryl ring. Non-limiting examples of “aryl” include indoline, tetrahydroquinoline, tetrahydroisoquinoline, and dihydrobenzofuran wherein the point of attachment for each group is on the aromatic ring.

For example

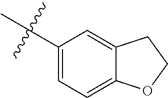 [SMILES: *c1ccc2c(c1)CCO2]

is an “aryl” group.

However,

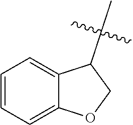 [SMILES: *C1COc2ccccc21]

is a“heterocycle” group.

1. In one embodiment a compound selected from Formula IL Formula IL, Formula III, Formula IV, Formula V, Formula VI, Formula VII, and Formula VIII is provided:

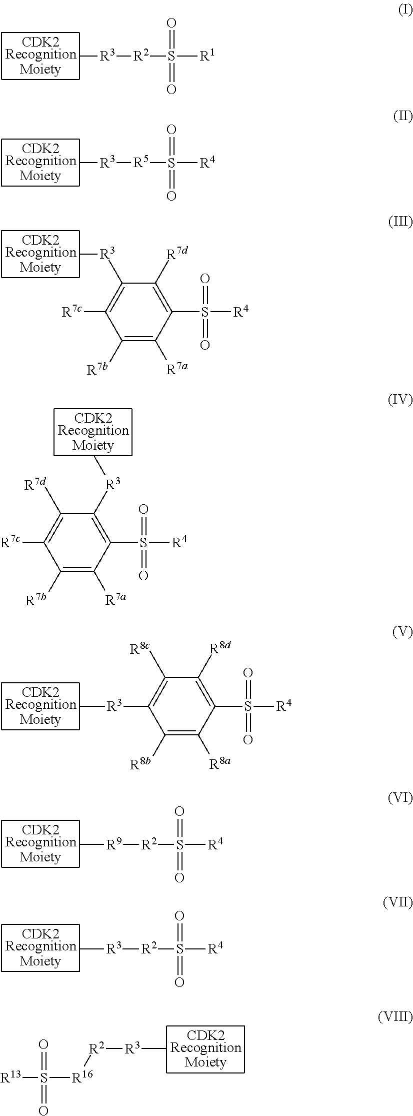

or a pharmaceutically acceptable salt, N-oxide, isotopic derivative, or prodrug thereof, wherein:

R¹ is selected from:
a)

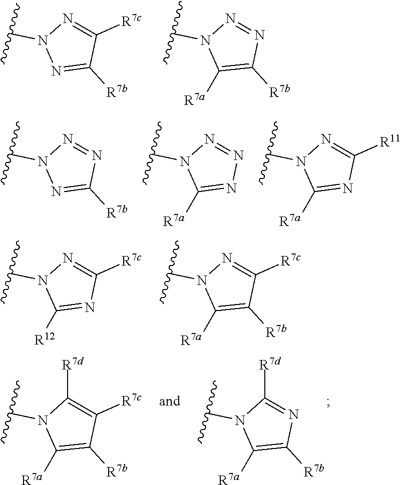

or
b) a bicyclic heteroaryl or tricyclic heteroaryl, each of which is optionally substituted as allowed by valence with 1, 2, 3, or 4 substituents selected from R⁷;

R² is independently selected at each instance from bond, alkyl, alkenyl, haloalkyl, cycloalkyl, aryl, heterocycle, —S—, —O—, —NR⁶—, —(CH₂)ₚ—C(O)—, —(CH₂)ₚ—C(O)—NR⁶—, —(CH₂CH₂O)ₚ—, —(OCH₂CH₂)ₚ—, —C(O)—, —NR⁶C(O)—, —NR⁶C(O)NR⁶—, —C(O)NR⁶—, —OC(O)NR⁶—, —NR⁶S(O)₂NR⁶—, —S(O)₂NR⁶—, aryl-C(O)—NR⁶—, heteroaryl-C(O)—NR⁶—, bicycle, tricycle, and heteroaryl, each of which is optionally substituted as allowed by valence with 1, 2, 3, or 4 substituents selected from R⁷;

R³ is independently selected at each instance from bond, alkyl, alkenyl, haloalkyl, cycloalkyl, aryl, heterocycle, —S—, —O—, —NR⁶—, —(CH₂)ₚ—C(O)—, —(CH₂)ₚ—C(O)—NR⁶—, —(CH₂CH₂O)ₚ—, —(OCH₂CH₂)ₚ—, —C(O)—, —NR⁶C(O)—, —NR⁶C(O)NR⁶—, —C(O)NR⁶—, —OC(O)NR⁶—, —NR⁶S(O)₂NR⁶—, —S(O)₂NR⁶—, aryl-C(O)—NR⁶—, heteroaryl-C(O)—NR⁶—, bicycle, tricycle, and heteroaryl, each of which is optionally substituted as allowed by valence with 1, 2, 3, or 4 substituents selected from R⁷;

and wherein R² and R³ are selected such that a suitably stable and suitably nontoxic compound for in vivo administration in a host is achieved, and in certain embodiments, in a way that avoids undesired repetition of atoms or moieties, such as in nonlimiting examples, S, O, or a combination thereof (i.e., that would otherwise form a disulfide or peroxide bond), as well known to skilled artisans;

p is independently selected from 1, 2, 3, 4, 5, and 6;

R⁴ is a heteroaryl group, where the bond to the sulfur atom is through one of the nitrogen atoms present in the cycle, and each heteroaryl is optionally substituted as allowed by valence with 1, 2, 3, or 4 substituents selected from R⁷;

R⁵ is selected from alkyl, alkenyl, haloalkyl, cycloalkyl, naphthyl, heterocycle, —S—, —O—, —NR⁶—, —(CH₂)ₚ—C(O)—, —(CH₂)ₚ—C(O)—NR⁶—, —(CH₂CH₂O)ₚ—, —(OCH₂CH₂)ₚ—, —C(O)—, —NR⁶C(O)—, —NR⁶C(O)NR⁶—, —C(O)NR⁶—, —OC(O)NR⁶—, —NR⁶S(O)₂NR⁶—, —S(O)₂NR⁶—, heteroaryl-C(O)—NR⁶—, bicycle, tricycle, and heteroaryl, each of which is optionally substituted as allowed by valence with 1, 2, 3, or 4 substituents selected from R⁷;

and wherein R³ and R⁵ are selected such that a suitably stable and suitably nontoxic compound for in vivo administration in a host is achieved, and in certain embodiments, in a way that avoids undesired repetition of atoms or moieties, such as in nonlimiting examples, S, O, or a combination thereof (i.e., that would otherwise form a disulfide or peroxide bond), as well known to skilled artisans

R⁶ is independently selected at each instance from hydrogen, alkyl, haloalkyl, cycloalkyl, aryl, heterocycle, and heteroaryl; each of which except hydrogen is optionally substituted as allowed by valence with 1, 2, 3, or 4 substituents selected from R¹⁸;

R⁷, R^{7a}, R^{7b}, R^{7c}, and R^{7d} are independently selected at each instance from hydrogen, halogen, alkyl, haloalkyl, alkenyl, cycloalkyl, heterocycle, aryl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, —S(O)R⁶, —S(O)₂R⁶, —S(O)OR⁶, —S(O)₂OR⁶, —S(O)N(R⁶)₂, S(O)₂N(R⁶)₂, ═O, and —SR⁶, wherein each alkyl, haloalkyl, alkenyl, cycloalkyl, heterocycle, aryl, and heteroaryl is optionally substituted as allowed by valence with 1, 2, 3, or 4 substituents selected from R¹⁷;

R¹⁷ is independently selected in each instance from hydrogen, halogen, alkyl, haloalkyl, alkenyl, cycloalkyl, heterocycle, aryl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, —S(O)R⁶, —S(O)₂R⁶, —S(O)OR⁶, —S(O)₂OR⁶, —S(O)N(R⁶)₂, S(O)₂N(R⁶)₂, ═O, and —SR⁶;

R¹⁸ is independently selected in each instance from hydrogen, halogen, alkyl, haloalkyl, alkenyl, cycloalkyl, heterocycle, aryl, heteroaryl, cyano, nitro, —C(O)R¹⁹, —OC(O)R¹⁹, —NR¹⁹C(O)R¹⁹, —C(O)OR¹⁹, —OC(O)OR¹⁹, —NR¹⁹C(O)OR¹⁹, —C(O)N(R¹⁹)₂, —OC(O)N(R¹⁹)₂, —NR⁹C(O)N(R¹⁹)₂, —OR⁹, —N(R¹⁹)₂, —S(O)R⁹, —S(O)₂R⁹, —S(O)OR⁹, —S(O)₂OR¹⁹, —S(O)N(R¹⁹)₂, S(O)₂N(R¹⁹)₂, ═O, and —SR¹⁹;

R¹⁹ is independently selected at each instance from hydrogen, alkyl, haloalkyl, cycloalkyl, aryl, heterocycle, and heteroaryl;

R^{8a}, R^{8b}, R^{8c}, and R^{8d} are independently selected at each instance from R⁷ and R¹² wherein at least one of R^{8a}, R^{8b}, R^{8c}, and R^{8d} is R¹²;

R⁹ is selected from alkyl, alkenyl, haloalkyl, cycloalkyl, heterocycle, —NR⁶C(O)—, —NR⁶C(O)NR⁶—, —C(O)NR⁶—, —OC(O)NR⁶—, —NR⁶S(O)₂NR⁶—, —S(O)₂NR⁶—, bicycle, tricycle, and heteroaryl, each of which is optionally substituted as allowed by valence with 1, 2, 3, or 4 substituents selected from R⁷;

R¹¹ is selected from hydrogen, halogen, alkyl, haloalkyl, alkenyl, cycloalkyl, heterocycle, naphthyl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, and —SR⁶, wherein each alkyl, haloalkyl, alkenyl, cycloalkyl, heterocycle, aryl, and heteroaryl optionally substituted as allowed by valence with 1, 2, 3, or 4 substituents selected from R⁷; and

R¹² is independently selected from halogen, alkyl, haloalkyl, alkenyl, cycloalkyl, heterocycle, aryl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, —S(O)R⁶, —S(O)₂R⁶, —S(O)OR⁶, —S(O)₂OR⁶, —S(O)N(R⁶)₂, S(O)₂N(R⁶)₂, ═O, and —SR⁶, wherein each alkyl, haloalkyl, alkenyl, cycloalkyl, heterocycle, aryl, and heteroaryl is optionally substituted as allowed by valence with 1, 2, 3, or 4 substituents selected from R⁷; and

R¹³ is selected from hydrogen, alkyl, haloalkyl, cycloalkyl, heterocycle, heteroaryl, aryl, —OR⁶, —N(R⁶)₂, —C(O)R⁶, —NR⁶C(O)R⁶, —C(O)N(R⁶)₂, and —NR⁶C(O)N(R⁶)₂, each of which except hydrogen is optionally substituted as allowed by valence with 1, 2, 3, or 4 substituents selected from R⁷;

R¹⁶ is a heteroaryl group, where the bond to the sulfur atom is through one of the nitrogen atoms present in the cycle, and R¹⁶ is optionally substituted as allowed by valence with 1, 2, 3, or 4 substituents selected from R⁷;

CDK2 Recognition Moiety is a molecule which can bind to or otherwise anchors to CDK2.

2. The compound of embodiment 1, wherein the compound is of Formula:

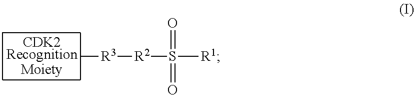

or a pharmaceutically acceptable salt thereof.

3. The compound of embodiment 1, wherein the compound is of Formula:

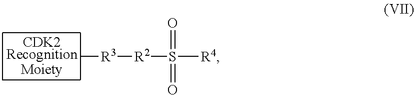

or a pharmaceutically acceptable salt thereof.

4. The compound of embodiment 1, wherein the compound is of Formula:

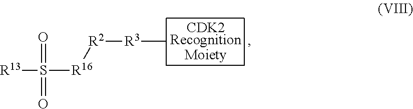

or a pharmaceutically acceptable salt thereof.

5. The compound of embodiment 4, wherein R¹³ is phenyl optionally substituted as allowed by valence with 1, 2, 3, or 4 substituents selected from R⁷.

6. The compound of embodiment 4, wherein R³ is alkyl optionally substituted as allowed by valence with 1, 2, 3, or 4 substituents selected from R⁷.

7. The compound of embodiment 4, wherein R³ is cycloalkyl optionally substituted as allowed by valence with 1, 2, 3, or 4 substituents selected from R⁷.

8. The compound of embodiment 4, wherein R³ is aryl optionally substituted as allowed by valence with 1, 2, 3, or 4 substituents selected from R⁷.

9. The compound of embodiment 4, wherein R³ is cyclopropyl optionally substituted as allowed by valence with 1, 2, 3, or 4 substituents selected from R⁷.

10. The compound of embodiment 4, wherein R³ is heterocycle optionally substituted as allowed by valence with 1, 2, 3, or 4 substituents selected from R⁷.

11. The compound of embodiment 4, wherein R³ is haloalkyl optionally substituted as allowed by valence with 1, 2, 3, or 4 substituents selected from R⁷.

12. The compound of any one of embodiments 5-11, wherein R¹⁶ is a triazole.

13. The compound of any one of embodiments 5-12, wherein R¹⁶ is

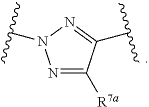

14. The compound of any one of embodiments 5-12, wherein R¹⁶ is

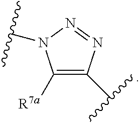

15. The compound of any one of embodiments 5-11, wherein R¹⁶ is

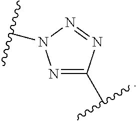 [SMILES: *c1nnn(*)n1]

16. The compound of any one of embodiments 5-11, wherein R¹⁶ is

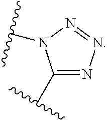 [SMILES: *c1nnnn1*]

17. The compound of any one of embodiments 5-12, wherein R¹⁶ is

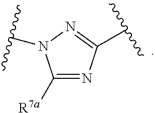

18. The compound of any one of embodiments 5-11, wherein R¹⁶ is

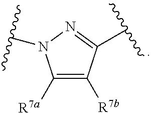

19. The compound of any one of embodiments 5-11, wherein R¹⁶ is

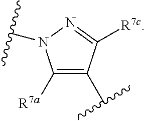

20. The compound of any one of embodiments 5-11, wherein R¹⁶ is

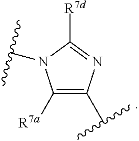

21. The compound of embodiment 1, wherein the compound is of Formula:

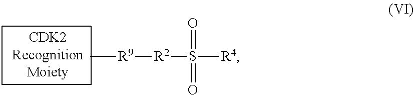

or a pharmaceutically acceptable salt thereof.

22. The compound of embodiment 21, wherein R⁹ is selected alkyl, alkenyl, haloalkyl, cycloalkyl, bicycle, tricycle, and heteroaryl, each of which except bond is optionally substituted as allowed by valence with 1, 2, 3, or 4 substituents selected from R⁷.

23. The compound of embodiment 22, wherein R⁹ is alkyl optionally substituted as allowed by valence with 1, 2, 3, or 4 substituents selected from R⁷.

24. The compound of embodiment 22, wherein R⁹ is alkenyl optionally substituted as allowed by valence with 1, 2, 3, or 4 substituents selected from R⁷.

25. The compound of embodiment 22, wherein R⁹ is haloalkyl optionally substituted as allowed by valence with 1, 2, 3, or 4 substituents selected from R⁷.

26. The compound of embodiment 22, wherein R⁹ is cycloalkyl optionally substituted as allowed by valence with 1, 2, 3, or 4 substituents selected from R⁷.

27. The compound of embodiment 22, wherein R⁹ is heteroaryl optionally substituted as allowed by valence with 1, 2, 3, or 4 substituents selected from R⁷.

28. The compound of embodiment 22, wherein R⁹ is bicycle optionally substituted as allowed by valence with 1, 2, 3, or 4 substituents selected from R⁷.

29. The compound of any one of embodiments 1-20, wherein R³ is bond.

30. The compound of any one of embodiments 1-20, wherein R³ is phenyl optionally substituted as allowed by valence with 1, 2, 3, or 4 substituents selected from R⁷.

31. The compound of any one of embodiments 1-20, wherein R³ is alkyl optionally substituted as allowed by valence with 1, 2, 3, or 4 substituents selected from R⁷.

32. The compound of any one of embodiments 1-20, wherein R³ is alkenyl optionally substituted as allowed by valence with 1, 2, 3, or 4 substituents selected from R⁷.

33. The compound of any one of embodiments 1-20, wherein R³ is haloalkyl optionally substituted as allowed by valence with 1, 2, 3, or 4 substituents selected from R⁷.

34. The compound of any one of embodiments 1-20, wherein R³ is cycloalkyl optionally substituted as allowed by valence with 1, 2, 3, or 4 substituents selected from R⁷.

35. The compound of any one of embodiments 1-20, wherein R³ is aryl optionally substituted as allowed by valence with 1, 2, 3, or 4 substituents selected from R⁷.

36. The compound of any one of embodiments 1-20, wherein R³ is heteroaryl optionally substituted as allowed by valence with 1, 2, 3, or 4 substituents selected from R⁷.

37. The compound of any one of embodiments 1-20, wherein R³ is bicycle optionally substituted as allowed by valence with 1, 2, 3, or 4 substituents selected from R⁷.

38. The compound of any one of embodiments 1-37, wherein R² is selected from bond, alkyl, alkenyl, haloalkyl, cycloalkyl, aryl, bicycle, tricycle, and heteroaryl, each of which except bond is optionally substituted as allowed by valence with 1, 2, 3, or 4 substituents selected from R⁷.

39. The compound of any one of embodiments 1-38, wherein R² is bond.

40. The compound of any one of embodiments 1-38, wherein R² is phenyl optionally substituted as allowed by valence with 1, 2, 3, or 4 substituents selected from R⁷.

41. The compound of any one of embodiments 1-38, wherein R² is alkyl optionally substituted as allowed by valence with 1, 2, 3, or 4 substituents selected from R⁷.

42. The compound of any one of embodiments 1-38, wherein R² is alkenyl optionally substituted as allowed by valence with 1, 2, 3, or 4 substituents selected from R⁷.

43. The compound of any one of embodiments 1-38, wherein R² is haloalkyl optionally substituted as allowed by valence with 1, 2, 3, or 4 substituents selected from R⁷.

44. The compound of any one of embodiments 1-38, wherein R² is cycloalkyl optionally substituted as allowed by valence with 1, 2, 3, or 4 substituents selected from R⁷.

45. The compound of any one of embodiments 1-38, wherein R² is aryl optionally substituted as allowed by valence with 1, 2, 3, or 4 substituents selected from R⁷.

46. The compound of any one of embodiments 1-38, wherein R² is heteroaryl optionally substituted as allowed by valence with 1, 2, 3, or 4 substituents selected from R⁷.

47. The compound of any one of embodiments 1-38, wherein R² is bicycle optionally substituted as allowed by valence with 1, 2, 3, or 4 substituents selected from R⁷.

48. The compound of any one of embodiments 1-47, wherein R² is not substituted.

49. The compound of any one of embodiments 1-47, wherein R² is substituted as allowed by valence with 1 substituent selected from R⁷.

50. The compound of any one of embodiments 1-47, wherein R² is substituted as allowed by valence with 2 substituents selected from R⁷.

51. The compound of any one of embodiments 1-47, wherein R² is substituted as allowed by valence with 3 substituents selected from R⁷.

52. The compound of embodiment 1, wherein the compound is of Formula:

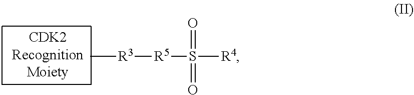

or a pharmaceutically acceptable salt thereof.

53. The compound of embodiment 1 wherein the compound is Formula:

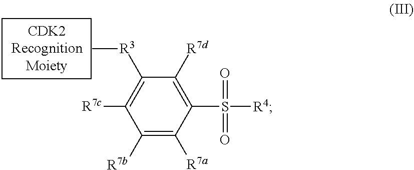

or a pharmaceutically acceptable salt thereof.

54. The compound of embodiment 1 wherein the compound is Formula:

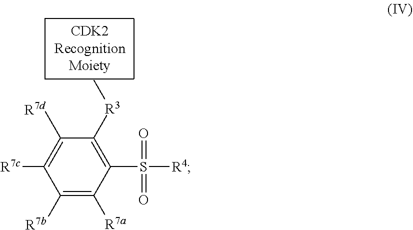

or a pharmaceutically acceptable salt thereof.

55. The compound of embodiment 1 wherein the compound is Formula:

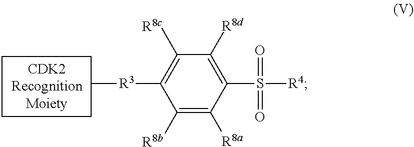

or a pharmaceutically acceptable salt thereof.

56. The compound of any one of embodiments 52-55, wherein R³ is bond.

57. The compound of any one of embodiments 52-55, wherein R³ is phenyl optionally substituted as allowed by valence with 1, 2, 3, or 4 substituents selected from R⁷.

58. The compound of any one of embodiments 52-55, wherein R³ is alkyl optionally substituted as allowed by valence with 1, 2, 3, or 4 substituents selected from R⁷.

59. The compound of any one of embodiments 52-55, wherein R³ is alkenyl optionally substituted as allowed by valence with 1, 2, 3, or 4 substituents selected from R⁷.

60. The compound of any one of embodiments 52-55, wherein R³ is haloalkyl optionally substituted as allowed by valence with 1, 2, 3, or 4 substituents selected from R⁷.

61. The compound of any one of embodiments 52-55, wherein R³ is cycloalkyl optionally substituted as allowed by valence with 1, 2, 3, or 4 substituents selected from R⁷.

62. The compound of any one of embodiments 52-55, wherein R³ is aryl optionally substituted as allowed by valence with 1, 2, 3, or 4 substituents selected from R⁷.

63. The compound of any one of embodiments 52-55, wherein R³ is heteroaryl optionally substituted as allowed by valence with 1, 2, 3, or 4 substituents selected from R⁷.

64. The compound of any one of embodiments 52-55, wherein R³ is bicycle optionally substituted as allowed by valence with 1, 2, 3, or 4 substituents selected from R⁷.

65. The compound of any one of embodiments 1-3, or 21-64, wherein R¹ and R⁴ are

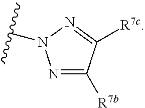

66. The compound of any one of embodiments 1-3, or 21-64, wherein R¹ and R⁴ are

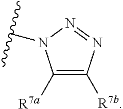

67. The compound of any one of embodiments 1-3, or 21-64, wherein R¹ and R⁴ are

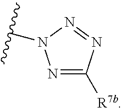

68. The compound of any one of embodiments 1-3, or 21-64, wherein R¹ and R⁴ are

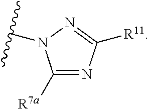

69. The compound of any one of embodiments 1-3, or 21-64, wherein R¹ and R⁴ are

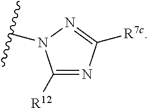

70. The compound of any one of embodiments 1-3, or 21-64, wherein R¹ and R⁴ are

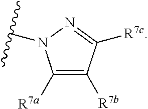

71. The compound of any one of embodiments 1-3, or 21-64, wherein R¹ and R⁴ are

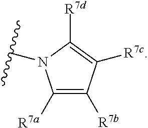

72. The compound of any one of embodiments 1-3, or 21-64, wherein R¹ and R⁴ are

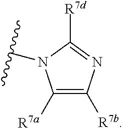

73. The compound of any one of embodiments 1-3, or 21-64, wherein R¹ and R⁴ are a bicyclic heteroaryl which is optionally substituted as allowed by valence with 1, 2, 3, or 4 substituents selected from R⁷.

74. The compound of any one of embodiments 1-3, or 21-64, wherein R⁴ is a heteroaryl group, where the bond to the sulfur atom is through the nitrogen present in the cycle, and each heteroaryl is optionally substituted as allowed by valence with 1, 2, 3, or 4 substituents selected from R⁷.

75. The compound of any one of embodiments 1-74, wherein R⁶ is independently selected at each instance from alkyl, haloalkyl, cycloalkyl, aryl, heterocycle, and heteroaryl; each of which is optionally substituted as allowed by valence with 1, 2, 3, or 4 substituents selected from R¹⁸.

76. The compound of embodiment 75, wherein R⁶ is alkyl optionally substituted as allowed by valence with 1, 2, 3, or 4 substituents selected from R¹⁸.

77. The compound of embodiment 75, wherein R⁶ is haloalkyl optionally substituted as allowed by valence with 1, 2, 3, or 4 substituents selected from R¹⁸.

78. The compound of embodiment 75, wherein R⁶ is cycloalkyl optionally substituted as allowed by valence with 1, 2, 3, or 4 substituents selected from R¹⁸.

79. The compound of embodiment 75, wherein R⁶ is aryl optionally substituted as allowed by valence with 1, 2, 3, or 4 substituents selected from R¹⁸.

80. The compound of embodiment 75, wherein R⁶ is heterocycle optionally substituted as allowed by valence with 1, 2, 3, or 4 substituents selected from R¹⁸.

81. The compound of embodiment 75, wherein R⁶ is heteroaryl optionally substituted as allowed by valence with 1, 2, 3, or 4 substituents selected from R¹⁸.

82. The compound of any one of embodiments 75-81, wherein R⁶ is not substituted with R¹⁸.

83. The compound of any one of embodiments 75-81, wherein R⁶ is substituted with 1 substituent selected from R¹⁸.

84. The compound of any one of embodiments 75-81, wherein R⁶ is substituted with 2 substituents independently selected from R¹⁸.

85. The compound of any one of embodiments 75-81, wherein R⁶ is substituted with 3 substituents independently selected from R¹⁸.

86. The compound of any one of embodiments 1-85, wherein R⁷, R^{7a}, R^{7b}, R^{7c}, and R^{7d} are independently selected at each instance from hydrogen, halogen, alkyl, haloalkyl, alkenyl, cycloalkyl, heterocycle, aryl, heteroaryl, cyano, and nitro, wherein each alkyl, haloalkyl, alkenyl, cycloalkyl, heterocycle, aryl, and heteroaryl is optionally substituted as allowed by valence with 1, 2, 3, or 4 substituents selected from R¹⁷

87. The compound of any one of embodiments 1-86, wherein one of R⁷, R^{7a}, R^{7b}, R^{7c}, and R^{7d} is alkyl optionally substituted as allowed by valence with 1, 2, 3, or 4 substituents selected from R¹⁷.

88. The compound of any one of embodiments 1-86, wherein one of R⁷, R^{7a}, R^{7b}, R^{7c}, and R^{7d} is haloalkyl optionally substituted as allowed by valence with 1, 2, 3, or 4 substituents selected from R¹⁷.

89. The compound of any one of embodiments 1-86, wherein one of R⁷, R^{7a}, R^{7b}, R^{7c}, and R^{7d} is phenyl optionally substituted as allowed by valence with 1, 2, 3, or 4 substituents selected from R¹⁷.

90. The compound of any one of embodiments 1-86, wherein one of R⁷, R^{7a}, R^{7b}, R^{7c}, and R^{7d} is heteroaryl optionally substituted as allowed by valence with 1, 2, 3, or 4 substituents selected from R¹⁷.

91. The compound of any one of embodiments 1-86, wherein one of R⁷, R^{7a}, R^{7b}, R^{7c}, and R^{7d} is haloalkyl optionally substituted as allowed by valence with 1, 2, 3, or 4 substituents selected from R¹⁷.

92. The compound of any one of embodiments 1-86, wherein one of R⁷, R^{7a}, R^{7b}, R^{7c}, and R^{7d} is heterocycle optionally substituted as allowed by valence with 1, 2, 3, or 4 substituents selected from R¹⁷.

93. The compound of any one of embodiments 1-86, wherein one of R⁷, R^{7a}, R^{7b}, R^{7c}, and R^{7d} is cycloalkyl optionally substituted as allowed by valence with 1, 2, 3, or 4 substituents selected from R¹⁷.

94. The compound of any one of embodiments 87-93, wherein R⁷, R^{7a}, R^{7b}, R^{7c}, and R^{7d} are not substituted.

95. The compound of any one of embodiments 87-93, wherein R⁷, R^{7a}, R^{7b}, R^{7c}, and R^{7d} are optionally substituted with 1 or 2 substituents selected from R¹⁷.

96. The compound of any one of embodiments 1-86, wherein one of R⁷, R^{7a}, R^{7b}, R^{7c}, and R^{7d} is halogen.

97. The compound of any one of embodiments 1-86, wherein one of R⁷, R^{7a}, R^{7b}, R^{7c}, and R^{7d} is —OR⁶.

98. The compound of any one of embodiments 1-86, wherein one of R⁷, R^{7a}, R^{7b}, R^{7c}, and R^{7d} is ═O.

99. The compound of any one of embodiments 1-86, wherein one of R⁷, R^{7a}, R^{7b}, R^{7c}, and R^{7d} is cyano.

100. The compound of any one of embodiments 1-86, wherein one of R⁷, R^{7a}, R^{7b}, R^{7c}, and R^{7d} is nitro.

101. The compound of any one of embodiments 1-100, wherein R¹⁷ is selected in each instance from halogen, alkyl, haloalkyl, alkenyl, and cyano.

102. In one embodiment a pharmaceutical composition comprising a compound of any one of embodiments 1-101 or a pharmaceutically acceptable salt thereof and a pharmaceutically acceptable excipient is provided.

103. In one embodiment a method of treating a disorder mediated by CDK2 comprising administering an effective amount of a compound of any one of embodiments 1-102 or a pharmaceutically acceptable salt thereof to a patient in need thereof is provided.

104. The method of embodiment 103 wherein the patient is a human.

In other embodiments a compound, pharmaceutical composition or method described below is provided:

1. A compound of Formula:

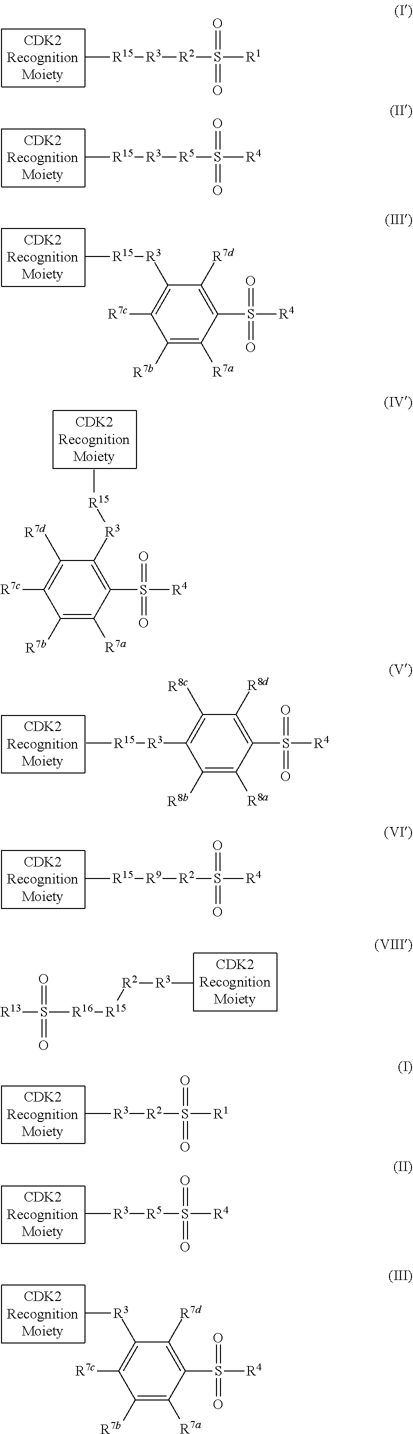

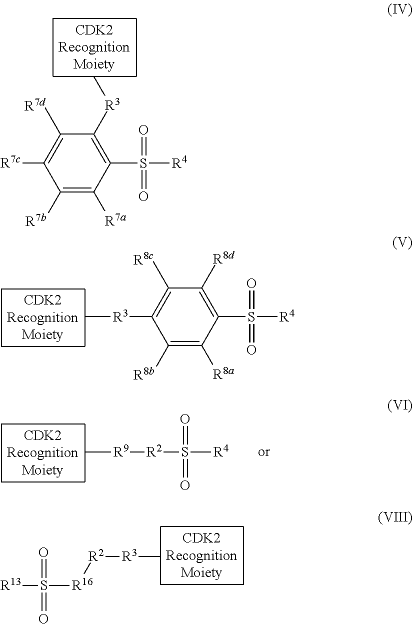

or a pharmaceutically acceptable salt thereof,
wherein:

R¹ is selected from:
a)

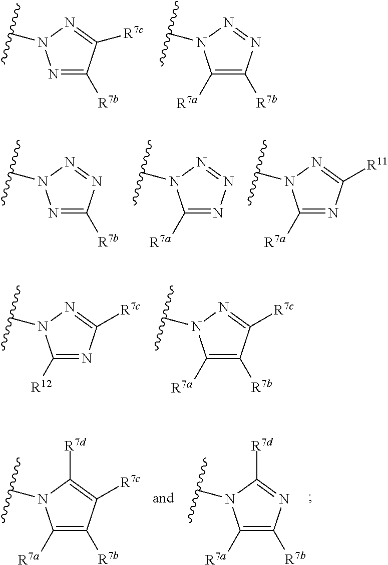

or
b) a bicyclic heteroaryl or tricyclic heteroaryl, optionally substituted as allowed by valence with 1, 2, 3, or 4 substituents selected from R⁷;

R² is independently selected at each instance from bond, alkyl, alkenyl, haloalkyl, cycloalkyl, aryl, heterocycle, —S—, —O—, —NR⁶—, —(CH₂)ₚ—C(O)—, —(CH₂)ₚ—C(O)—NR⁶—, —(CH₂CH₂O)ₚ—, —(OCH₂CH₂)ₚ—, —C(O)—, —NR⁶C(O)—, —NR⁶C(O)NR⁶—, —C(O)NR⁶—, —OC(O)NR⁶—, —NR⁶S(O)₂NR⁶—, —S(O)₂NR⁶—, aryl-C(O)—NR⁶—, heteroaryl-C(O)—NR⁶—, bicycle, tricycle, and heteroaryl, each of which is optionally substituted as allowed by valence with 1, 2, 3, or 4 substituents selected from R⁷;

R³ is independently selected at each instance from bond, alkyl, alkenyl, haloalkyl, cycloalkyl, aryl, heterocycle, —S—, —O—, —NR⁶—, —S-alkyl-, —O-alkyl-, —NR⁶-alkyl-, -alkyl-C(O)—, -alkyl-C(O)-alkyl-, -alkyl-C(O)—NR⁶-alkyl-, —C(O)—NR⁶-alkyl-, -alkyl-C(O)—NR⁶—, -alkyl-C(O)—O-alkyl-, —C(O)—O-alkyl-, -alkyl-C(O)—O—, —C(O)—, —NR⁶C(O)NR⁶—, —C(O)NR⁶—, —OC(O)NR⁶—, —NR⁶S(O)₂NR⁶—, —S(O)₂NR⁶—, bicycle, tricycle, and heteroaryl, each of which except bond is optionally substituted as allowed by valence with 1, 2, 3, or 4 substituents selected from R⁷;

and wherein R² and R³ are selected such that a suitably stable and suitably nontoxic compound for in vivo administration in a host is achieved;

each p is independently selected from 1, 2, 3, 4, 5, and 6;

R⁴ is a heteroaryl group, where the bond to the sulfur atom is through one of the nitrogen atoms present in the cycle, and each heteroaryl is optionally substituted as allowed by valence with 1, 2, 3, or 4 substituents selected from R⁷;

R⁵ is selected from alkyl, alkenyl, haloalkyl, cycloalkyl, naphthyl, heterocycle, —S—, —O—, —NR⁶—, —(CH₂)ₚ—C(O)—, —(CH₂)ₚ—C(O)—NR⁶—, —(CH₂CH₂O)ₚ—, —(OCH₂CH₂)ₚ—, —C(O)—, —NR⁶C(O)—, —NR⁶C(O)NR⁶—, —C(O)NR⁶—, —OC(O)NR⁶—, —NR⁶S(O)₂NR⁶—, —S(O)₂NR⁶—, heteroaryl-C(O)—NR⁶—, bicycle, tricycle, and heteroaryl, each of which is optionally substituted as allowed by valence with 1, 2, 3, or 4 substituents selected from R⁷;

and wherein R³ and R⁵ are selected such that a suitably stable and suitably nontoxic compound for in vivo administration in a host is achieved;

R⁶ is independently selected at each instance from hydrogen, alkyl, haloalkyl, cycloalkyl, aryl, heterocycle, and heteroaryl; each of which except hydrogen is optionally substituted as allowed by valence with 1, 2, 3, or 4 substituents selected from R¹⁸;

R⁷, R^{7a}, R^{7b}, R^{7c}, and R^{7d} are independently selected at each instance from hydrogen, halogen, alkyl, haloalkyl, alkenyl, cycloalkyl, heterocycle, aryl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, —S(O)R⁶, —S(O)₂R⁶, —S(O)OR⁶, —S(O)₂OR⁶, —S(O)N(R⁶)₂, S(O)₂N(R⁶)₂, ═O, and —SR⁶, wherein each alkyl, haloalkyl, alkenyl, cycloalkyl, heterocycle, aryl, and heteroaryl is optionally substituted as allowed by valence with 1, 2, 3, or 4 substituents selected from R¹⁷;

R¹⁵ is selected from the group consisting of alkyl, alkenyl, haloalkyl, cycloalkyl, aryl, heterocycle, —S—, —O—, —NR⁶—, —S-alkyl-, —O-alkyl-, —NR⁶-alkyl-, -alkyl-C(O)—, -alkyl-C(O)-alkyl-, -alkyl-C(O)—NR⁶-alkyl-, —C(O)—NR⁶-alkyl-, -alkyl-C(O)—NR⁶—, -alkyl-C(O)—O-alkyl-, —C(O)—O-alkyl-, -alkyl-C(O)—O—, —C(O)—, —NR⁶C(O)NR⁶—, —C(O)NR⁶—, —OC(O)NR⁶—, —NR⁶S(O)₂NR⁶—, —S(O)₂NR⁶—, bicycle, tricycle, and heteroaryl, each of which except bond is optionally substituted as allowed by valence with 1, 2, 3, or 4 substituents independently selected from R⁷;

R¹⁷ is independently selected at each instance from hydrogen, halogen, alkyl, haloalkyl, alkenyl, cycloalkyl, heterocycle, aryl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, —S(O)R⁶, —S(O)₂R⁶, —S(O)OR⁶, —S(O)₂OR⁶, —S(O)N(R)₂, S(O)₂N(R⁶)₂, ═O, and —SR⁶;

R¹⁸ is independently selected at each instance from hydrogen, halogen, alkyl, haloalkyl, alkenyl, cycloalkyl, heterocycle, aryl, heteroaryl, cyano, nitro, —C(O)R¹⁹, —OC(O)R¹⁹, —NR¹⁹C(O)R¹⁹, —C(O)OR¹⁹, —OC(O)OR¹⁹, —NR¹⁹C(O)OR¹⁹, —C(O)N(R¹⁹)₂, —OC(O)N(R¹⁹)₂, —NR¹⁹C(O)N(R¹⁹)₂, —OR¹⁹, —N(R¹⁹)₂, —S(O)R¹⁹, —S(O)₂R¹⁹, —S(O)OR¹⁹, —S(O)₂OR¹⁹, —S(O)N(R¹⁹)₂, S(O)₂N(R¹⁹)₂, ═O, and —SR¹⁹;

R¹⁹ is independently selected at each instance from hydrogen, alkyl, haloalkyl, cycloalkyl, aryl, heterocycle, and heteroaryl;

R^{8a}, R^{8b}, R^{8c}, and R^{8d} are independently selected at each instance from R⁷ and R¹² wherein at least one of R^{8a}, R^{8b}, R^{8c}, and R^{8d} is R¹²;

R⁹ is selected from alkyl, alkenyl, haloalkyl, cycloalkyl, heterocycle, -alkyl-C(O)—NR⁶-alkyl-, —C(O)—NR⁶-alkyl-, -alkyl-C(O)—NR⁶—, -alkyl-C(O)—O-alkyl-, —C(O)—O-alkyl-, -alkyl-C(O)—O—, —NR⁶C(O)—, —NR⁶C(O)NR⁶—, —C(O)NR⁶—, —OC(O)NR⁶—, —NR⁶S(O)₂NR⁶—, —S(O)₂NR⁶—, bicycle, tricycle, and heteroaryl, each of which is optionally substituted as allowed by valence with 1, 2, 3, or 4 substituents selected from R⁷;

R¹¹ is selected from hydrogen, halogen, alkyl, haloalkyl, alkenyl, cycloalkyl, heterocycle, naphthyl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, and —SR⁶, wherein each alkyl, haloalkyl, alkenyl, cycloalkyl, heterocycle, aryl, and heteroaryl is optionally substituted as allowed by valence with 1, 2, 3, or 4 substituents selected from R¹⁷;

R¹² is independently selected from halogen, alkyl, haloalkyl, alkenyl, cycloalkyl, heterocycle, aryl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, —S(O)R⁶, —S(O)₂R⁶, —S(O)OR⁶, —S(O)₂OR⁶, —S(O)N(R⁶)₂, S(O)₂N(R⁶)₂, ═O, and —SR⁶, wherein each alkyl, haloalkyl, alkenyl, cycloalkyl, heterocycle, aryl, and heteroaryl is optionally substituted as allowed by valence with 1, 2, 3, or 4 substituents selected from R¹⁷;

R¹³ is selected from hydrogen, alkyl, haloalkyl, cycloalkyl, heterocycle, heteroaryl, aryl, —OR⁶, —N(R⁶)₂, —C(O)R⁶, —NR⁶C(O)R⁶, —C(O)N(R⁶)₂, and —NR⁶C(O)N(R⁶)₂, each of which except hydrogen is optionally substituted as allowed by valence with 1, 2, 3, or 4 substituents selected from R⁷;

R¹⁶ is a heteroaryl group, where the bond to the sulfur atom is through one of the nitrogen atoms present in the cycle, and R¹⁶ is optionally substituted as allowed by valence with 1, 2, 3, or 4 substituents selected from R⁷; and

CDK2 Recognition Moiety is a molecule which can bind to or otherwise anchors too CDK2.

2. A compound of Formula:

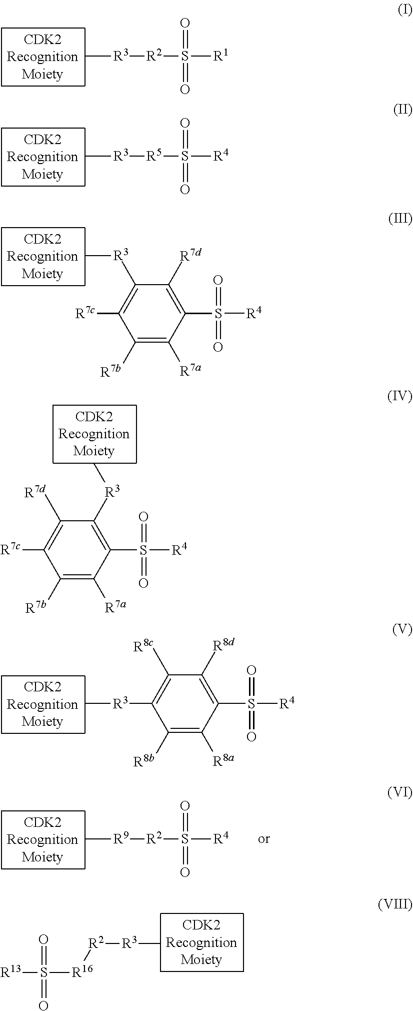

or a pharmaceutically acceptable salt thereof,
wherein:

R¹ is selected from:
a)

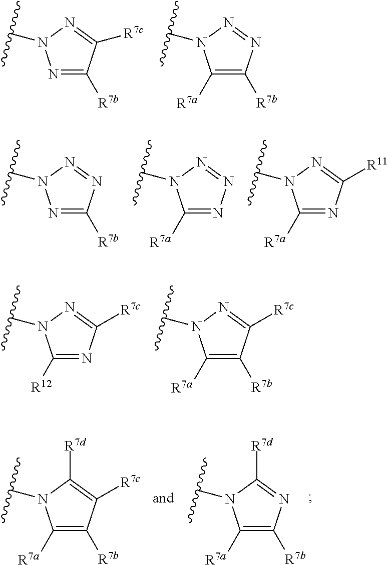

or
b) a bicyclic heteroaryl or tricyclic heteroaryl, each of which is optionally substituted as allowed by valence with 1, 2, 3, or 4 substituents selected from R⁷;

R² is independently selected at each instance from bond, alkyl, alkenyl, haloalkyl, cycloalkyl, aryl, heterocycle, —S—, —O—, —NR⁶—, —(CH₂)ₚ—C(O)—, —(CH₂)ₚ—C(O)—NR⁶—, —(CH₂CH₂O)ₚ—, —(OCH₂CH₂)ₚ—, —C(O)—, —NR⁶C(O)—, —NR⁶C(O)NR⁶—, —C(O)NR⁶—, —OC(O)NR⁶—, —NR⁶S(O)₂NR⁶—, —S(O)₂NR⁶—, aryl-C(O)—NR⁶—, heteroaryl-C(O)—NR⁶—, bicycle, tricycle, and heteroaryl, each of which is optionally substituted as allowed by valence with 1, 2, 3, or 4 substituents selected from R⁷;

R³ is independently selected at each instance from bond, alkyl, alkenyl, haloalkyl, cycloalkyl, aryl, heterocycle, —S—, —O—, —NR⁶—, —(CH₂)ₚ—C(O)—, —(CH₂)ₚ—C(O)—NR⁶—, —(CH₂CH₂O)ₚ—, —(OCH₂CH₂)ₚ—, —C(O)—, —NR⁶C(O)—, —NR⁶C(O)NR⁶—, —C(O)NR⁶—, —OC(O)NR⁶—, —NR⁶S(O)₂NR⁶—, —S(O)₂NR⁶—, aryl-C(O)—NR⁶—, heteroaryl-C(O)—NR⁶—, bicycle, tricycle, and heteroaryl, each of which is optionally substituted as allowed by valence with 1, 2, 3, or 4 substituents selected from R⁷;

and wherein R² and R³ are selected such that a suitably stable and suitably nontoxic compound for in vivo administration in a host is achieved;

p is independently selected from 1, 2, 3, 4, 5, and 6;

R⁴ is a heteroaryl group, where the bond to the sulfur atom is through one of the nitrogen atoms present in the cycle, and each heteroaryl is optionally substituted as allowed by valence with 1, 2, 3, or 4 substituents selected from R⁷;

R⁵ is selected from alkyl, alkenyl, haloalkyl, cycloalkyl, naphthyl, heterocycle, —S—, —O—, —NR⁶—, —(CH₂)ₚ—C(O)—, —(CH₂)ₚ—C(O)—NR⁶—, —(CH₂CH₂O)ₚ—, —(OCH₂CH₂)ₚ—, —C(O)—, —NR⁶C(O)—, —NR⁶C(O)NR⁶—, —C(O)NR⁶—, —OC(O)NR⁶—, —NR⁶S(O)₂NR⁶—, —S(O)₂NR⁶—, heteroaryl-C(O)—NR⁶—, bicycle, tricycle, and heteroaryl, each of which is optionally substituted as allowed by valence with 1, 2, 3, or 4 substituents selected from R⁷;

and wherein R³ and R⁵ are selected such that a suitably stable and suitably nontoxic compound for in vivo administration in a host is achieved;

R⁶ is independently selected at each instance from hydrogen, alkyl, haloalkyl, cycloalkyl, aryl, heterocycle, and heteroaryl; each of which except hydrogen is optionally substituted as allowed by valence with 1, 2, 3, or 4 substituents selected from R¹⁸;

R⁷, R^{7a}, R^{7b}, R^{7c}, and R^{7d} are independently selected at each instance from hydrogen, halogen, alkyl, haloalkyl, alkenyl, cycloalkyl, heterocycle, aryl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, —S(O)R⁶, —S(O)₂R⁶, —S(O)OR⁶, —S(O)₂OR⁶, —S(O)N(R⁶)₂, S(O)₂N(R⁶)₂, ═O, and —SR⁶, wherein each alkyl, haloalkyl, alkenyl, cycloalkyl, heterocycle, aryl, and heteroaryl is optionally substituted as allowed by valence with 1, 2, 3, or 4 substituents selected from R¹⁷;

R¹⁷ is independently selected in each instance from hydrogen, halogen, alkyl, haloalkyl, alkenyl, cycloalkyl, heterocycle, aryl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, —S(O)R⁶, —S(O)₂R⁶, —S(O)OR⁶, —S(O)₂OR⁶, —S(O)N(R⁶)₂, S(O)₂N(R⁶)₂, ═O, and —SR;

R¹⁸ is independently selected in each instance from hydrogen, halogen, alkyl, haloalkyl, alkenyl, cycloalkyl, heterocycle, aryl, heteroaryl, cyano, nitro, —C(O)R¹⁹, —OC(O)R¹⁹, —NR¹⁹C(O)R¹⁹, —C(O)OR¹⁹, —OC(O)OR¹⁹, —NR¹⁹C(O)OR¹⁹, —C(O)N(R¹⁹)₂, —OC(O)N(R¹⁹)₂, —NR¹⁹C(O)N(R¹⁹)₂, —OR¹⁹, —N(R¹⁹)₂, —S(O)R¹⁹, —S(O)₂R¹⁹, —S(O)OR¹⁹, —S(O)₂OR¹⁹, —S(O)N(R¹⁹)₂, S(O)₂N(R¹⁹)₂, ═O, and —SR¹⁹;

R¹⁹ is independently selected at each instance from hydrogen, alkyl, haloalkyl, cycloalkyl, aryl, heterocycle, and heteroaryl;

R^{8a}, R^{8b}, R^{8c}, and R^{8d} are independently selected at each instance from R⁷ and R¹² wherein at least one of R^{8a}, R^{8b}, R^{8c}, and R^{8d} is R¹²;

R⁹ is selected from alkyl, alkenyl, haloalkyl, cycloalkyl, heterocycle, —NR⁶C(O)—, —NR⁶C(O)NR⁶—, —C(O)NR⁶—, —OC(O)NR⁶—, —NR⁶S(O)₂NR⁶—, —S(O)₂NR⁶—, bicycle, tricycle, and heteroaryl, each of which is optionally substituted as allowed by valence with 1, 2, 3, or 4 substituents selected from R⁷;

R¹¹ is selected from hydrogen, halogen, alkyl, haloalkyl, alkenyl, cycloalkyl, heterocycle, naphthyl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, and —SR⁶, wherein

each alkyl, haloalkyl, alkenyl, cycloalkyl, heterocycle, aryl, and heteroaryl optionally substituted as allowed by valence with 1, 2, 3, or 4 substituents selected from R¹⁷; and

R¹² is independently selected from halogen, alkyl, haloalkyl, alkenyl, cycloalkyl, heterocycle, aryl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, —S(O)R⁶, —S(O)₂R⁶, —S(O)OR⁶, —S(O)₂OR⁶, —S(O)N(R⁶)₂, S(O)₂N(R⁶)₂, ═O, and —SR⁶, wherein each alkyl, haloalkyl, alkenyl, cycloalkyl, heterocycle, aryl, and heteroaryl is optionally substituted as allowed by valence with 1, 2, 3, or 4 substituents selected from R¹⁷;

R¹³ is selected from hydrogen, alkyl, haloalkyl, cycloalkyl, heterocycle, heteroaryl, aryl, —OR⁶, —N(R⁶)₂, —C(O)R⁶, —NR⁶C(O)R⁶, —C(O)N(R⁶)₂, and —NR⁶C(O)N(R⁶)₂, each of which except hydrogen is optionally substituted as allowed by valence with 1, 2, 3, or 4 substituents selected from R⁷;

R¹⁶ is a heteroaryl group, where the bond to the sulfur atom is through one of the nitrogen atoms present in the cycle, and R¹⁶ is optionally substituted as allowed by valence with 1, 2, 3, or 4 substituents selected from R⁷; and

CDK2 Recognition Moiety is a molecule which can bind to or otherwise anchors to CDK2.

3. The compound of embodiment 1 or embodiment 2, wherein the compound is of Formula:

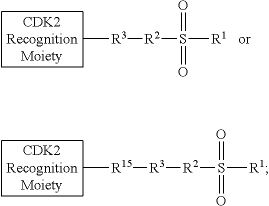

or a pharmaceutically acceptable salt thereof.

4. The compound of embodiment 1 or embodiment 2, wherein the compound is of Formula:

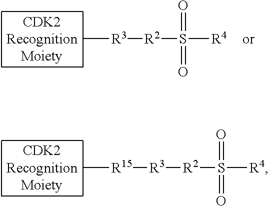

or a pharmaceutically acceptable salt thereof.

5. The compound of embodiment 1 or embodiment 2, wherein the compound is of Formula:

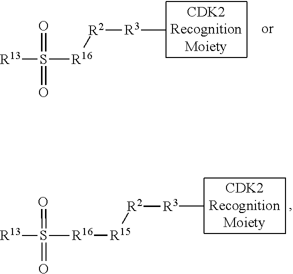

or a pharmaceutically acceptable salt thereof.

6. The compound of embodiment 5, wherein R¹³ is phenyl optionally substituted as allowed by valence with 1, 2, 3, or 4 substituents selected from R⁷.

7. The compound of embodiment 5, wherein R¹³ is alkyl optionally substituted as allowed by valence with 1, 2, 3, or 4 substituents selected from R⁷.

8. The compound of embodiment 5, wherein R¹³ is cycloalkyl optionally substituted as allowed by valence with 1, 2, 3, or 4 substituents selected from R⁷.

9. The compound of embodiment 5, wherein R¹³ is aryl optionally substituted as allowed by valence with 1, 2, 3, or 4 substituents selected from R⁷.

10. The compound of embodiment 5, wherein R¹³ is cyclopropyl optionally substituted as allowed by valence with 1, 2, 3, or 4 substituents selected from R⁷.

11. The compound of embodiment 5, wherein R¹³ is heterocycle optionally substituted as allowed by valence with 1, 2, 3, or 4 substituents selected from R⁷.

12. The compound of embodiment 5, wherein R¹³ is haloalkyl optionally substituted as allowed by valence with 1, 2, 3, or 4 substituents selected from R⁷.

13. The compound of any one of embodiments 5-12, wherein R¹⁶ is a triazole.

14. The compound of any one of embodiments 5-12, wherein R¹⁶ is

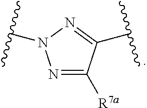

15. The compound of any one of embodiments 5-12, wherein R¹⁶ is

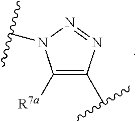

16. The compound of any one of embodiments 5-12, wherein R⁶ is

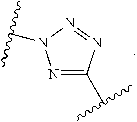 [SMILES: *c1nnn(*)n1]

17. The compound of any one of embodiments 5-12, wherein R¹⁶ is

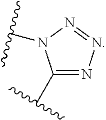 [SMILES: *c1nnnn1*]

18. The compound of any one of embodiments 5-12, wherein R¹⁶ is

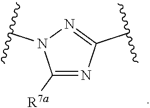

19. The compound of any one of embodiments 5-12, wherein R¹⁶ is

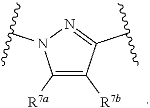

20. The compound of any one of embodiments 5-12, wherein R¹⁶ is

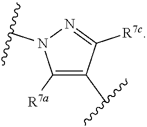

21. The compound of any one of embodiments 5-12, wherein R¹⁶ is

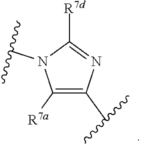

22. The compound of embodiment 1 or 2, wherein the compound is of Formula:

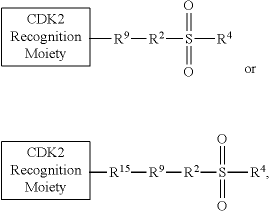

or a pharmaceutically acceptable salt thereof.

23. The compound of embodiment 22, wherein R⁹ is selected from alkyl, alkenyl, haloalkyl, cycloalkyl, bicycle, tricycle, and heteroaryl, each of which except bond is optionally substituted as allowed by valence with 1, 2, 3, or 4 substituents selected from R⁷.

24. The compound of embodiment 22, wherein R⁹ is alkyl optionally substituted as allowed by valence with 1, 2, 3, or 4 substituents selected from R⁷.

25. The compound of embodiment 22, wherein R⁹ is alkenyl optionally substituted as allowed by valence with 1, 2, 3, or 4 substituents selected from R⁷.

26. The compound of embodiment 22, wherein R⁹ is haloalkyl optionally substituted as allowed by valence with 1, 2, 3, or 4 substituents selected from R⁷.

27. The compound of embodiment 22, wherein R⁹ is cycloalkyl optionally substituted as allowed by valence with 1, 2, 3, or 4 substituents selected from R⁷.

28. The compound of embodiment 22, wherein R⁹ is heteroaryl optionally substituted as allowed by valence with 1, 2, 3, or 4 substituents selected from R⁷.

29. The compound of embodiment 22, wherein R⁹ is bicycle optionally substituted as allowed by valence with 1, 2, 3, or 4 substituents selected from R⁷.

30. The compound of any one of embodiments 1-29, wherein R³ is bond.

31. The compound of any one of embodiments 1-29, wherein R³ is phenyl optionally substituted as allowed by valence with 1, 2, 3, or 4 substituents selected from R⁷.

32. The compound of any one of embodiments 1-29, wherein R³ is alkyl optionally substituted as allowed by valence with 1, 2, 3, or 4 substituents selected from R⁷.

33. The compound of any one of embodiments 1-29, wherein R³ is alkenyl optionally substituted as allowed by valence with 1, 2, 3, or 4 substituents selected from R⁷.

34. The compound of any one of embodiments 1-29, wherein R³ is haloalkyl optionally substituted as allowed by valence with 1, 2, 3, or 4 substituents selected from R⁷.

35. The compound of any one of embodiments 1-29, wherein R³ is cycloalkyl optionally substituted as allowed by valence with 1, 2, 3, or 4 substituents selected from R⁷.

36. The compound of any one of embodiments 1-29, wherein R³ is aryl optionally substituted as allowed by valence with 1, 2, 3, or 4 substituents selected from R⁷.

37. The compound of any one of embodiments 1-29, wherein R³ is heteroaryl optionally substituted as allowed by valence with 1, 2, 3, or 4 substituents selected from R⁷.

38. The compound of any one of embodiments 1-29, wherein R³ is bicycle optionally substituted as allowed by valence with 1, 2, 3, or 4 substituents selected from R⁷.

39. The compound of any one of embodiments 1-38, wherein R² is selected from bond, alkyl, alkenyl, haloalkyl, cycloalkyl, aryl, bicycle, tricycle, and heteroaryl, each of which except bond is optionally substituted as allowed by valence with 1, 2, 3, or 4 substituents selected from R⁷.

40. The compound of any one of embodiments 1-38, wherein R² is bond.

41. The compound of any one of embodiments 1-38, wherein R² is phenyl optionally substituted as allowed by valence with 1, 2, 3, or 4 substituents selected from R⁷.

42. The compound of any one of embodiments 1-38, wherein R² is alkyl optionally substituted as allowed by valence with 1, 2, 3, or 4 substituents selected from R⁷.

43. The compound of any one of embodiments 1-38, wherein R² is alkenyl optionally substituted as allowed by valence with 1, 2, 3, or 4 substituents selected from R⁷.

44. The compound of any one of embodiments 1-38, wherein R² is haloalkyl optionally substituted as allowed by valence with 1, 2, 3, or 4 substituents selected from R⁷.

45. The compound of any one of embodiments 1-38, wherein R² is cycloalkyl optionally substituted as allowed by valence with 1, 2, 3, or 4 substituents selected from R⁷.

46. The compound of any one of embodiments 1-38, wherein R² is aryl optionally substituted as allowed by valence with 1, 2, 3, or 4 substituents selected from R⁷.

47. The compound of any one of embodiments 1-38, wherein R² is heteroaryl optionally substituted as allowed by valence with 1, 2, 3, or 4 substituents selected from R⁷.

48. The compound of any one of embodiments 1-38, wherein R² is bicycle optionally substituted as allowed by valence with 1, 2, 3, or 4 substituents selected from R⁷.

49. The compound of any one of embodiments 1-48, wherein R² is not substituted.

50. The compound of any one of embodiments 1-48, wherein R² is substituted as allowed by valence with 1 substituent selected from R⁷.

51. The compound of any one of embodiments 1-48, wherein R² is substituted as allowed by valence with 2 substituents selected from R⁷.

52. The compound of any one of embodiments 1-48, wherein R² is substituted as allowed by valence with 3 substituents selected from R⁷.

53. The compound of embodiment 1, wherein the compound is of Formula:

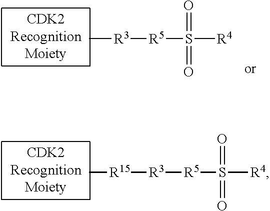

or a pharmaceutically acceptable salt thereof.

54. The compound of embodiment 1, wherein the compound is Formula:

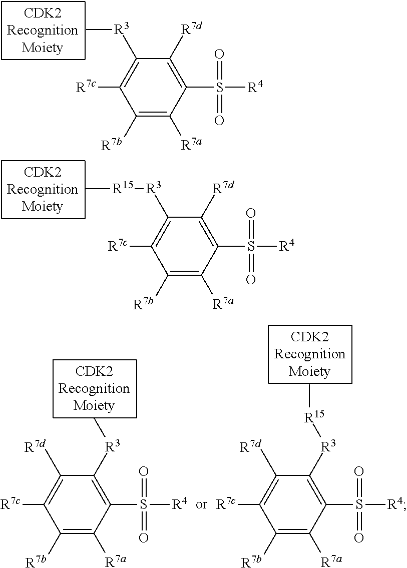

or a pharmaceutically acceptable salt thereof.

55. A compound of Formula

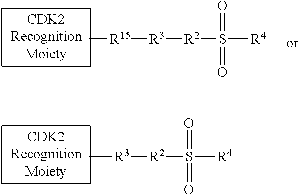

or a pharmaceutically acceptable salt thereof,
wherein:

R² is independently selected at each instance from bond, alkyl, alkenyl, haloalkyl, cycloalkyl, aryl, heterocycle, —S—, —O—, —NR⁶—, —(CH₂)ₚ—C(O)—, —(CH₂)ₚ—C(O)—NR⁶—, —(CH₂CH₂O)ₚ—, —(OCH₂CH₂)ₚ—, —C(O)—, —NR⁶C(O)—, —NR⁶C(O)NR⁶—, —C(O)NR⁶—, —OC(O)NR⁶—, —NR⁶S(O)₂NR⁶—, —S(O)₂NR⁶—, aryl-C(O)—NR⁶—, heteroaryl-C(O)—NR⁶—, bicycle, tricycle, and heteroaryl, each of which is optionally substituted as allowed by valence with 1, 2, 3, or 4 substituents selected from R⁷;

R³ is independently selected at each instance from bond, alkyl, alkenyl, haloalkyl, cycloalkyl, aryl, heterocycle, —S—, —O—, —NR⁶—, —S-alkyl-, —O-alkyl-, —NR⁶-alkyl-, -alkyl-C(O)—, -alkyl-C(O)-alkyl-, -alkyl-C(O)—NR⁶-alkyl-, —C(O)—NR⁶-alkyl-, -alkyl-C(O)—NR⁶—, -alkyl-C(O)—O-alkyl-, —C(O)—O-alkyl-, -alkyl-C(O)—O—, —C(O)—, —NR⁶C(O)NR⁶—, —C(O)NR⁶—, —OC(O)NR⁶—, —NR⁶S(O)₂NR⁶—, —S(O)₂NR⁶—, bicycle, tricycle, and heteroaryl, each of which except bond is optionally substituted as allowed by valence with 1, 2, 3, or 4 substituents selected from R⁷;

and wherein R² and R³ are selected such that a suitably stable and suitably nontoxic compound for in vivo administration in a host is achieved;

each p is independently selected from 1, 2, 3, 4, 5, and 6;

R⁴ is a heteroaryl group, where the bond to the sulfur atom is through one of the nitrogen atoms present in the cycle, and each heteroaryl is optionally substituted as allowed by valence with 1, 2, 3, or 4 substituents selected from R⁷;

R⁶ is independently selected at each instance from hydrogen, alkyl, haloalkyl, cycloalkyl, aryl, heterocycle, and heteroaryl; each of which except hydrogen is optionally substituted as allowed by valence with 1, 2, 3, or 4 substituents selected from R¹⁸;

R⁷ is independently selected at each instance from hydrogen, halogen, alkyl, haloalkyl, alkenyl, cycloalkyl, heterocycle, aryl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, —S(O)R⁶, —S(O)₂R⁶, —S(O)OR⁶, —S(O)₂OR⁶, —S(O)N(R⁶)₂, S(O)₂N(R⁶)₂, ═O, and —SR⁶, wherein each alkyl, haloalkyl, alkenyl, cycloalkyl, heterocycle, aryl, and heteroaryl is optionally substituted as allowed by valence with 1, 2, 3, or 4 substituents selected from R⁷;

R¹⁷ is selected from the group consisting of alkyl, alkenyl, haloalkyl, cycloalkyl, aryl, heterocycle, —S—, —O—, —NR⁶—, —S-alkyl-, —O-alkyl-, —NR⁶-alkyl-, -alkyl-C(O)—, -alkyl-C(O)-alkyl-, -alkyl-C(O)—NR⁶-alkyl-, —C(O)—NR⁶-alkyl-, -alkyl-C(O)—NR⁶—, -alkyl-C(O)—O-alkyl-, —C(O)—O-alkyl-, -alkyl-C(O)—O—, —C(O)—, —NR⁶C(O)NR⁶—, —C(O)NR⁶—, —OC(O)NR⁶—, —NR⁶S(O)₂NR⁶—, —S(O)₂NR⁶—, bicycle, tricycle, and heteroaryl, each of which except bond is optionally substituted as allowed by valence with 1, 2, 3, or 4 substituents independently selected from R⁷;

R¹⁷ is independently selected in each instance from hydrogen, halogen, alkyl, haloalkyl, alkenyl, cycloalkyl, heterocycle, aryl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, —S(O)R⁶, —S(O)₂R⁶, —S(O)OR⁶, —S(O)₂OR⁶, —S(O)N(R⁶)₂, S(O)₂N(R⁶)₂, ═O, and —SR⁶;

R¹⁸ is independently selected in each instance from hydrogen, halogen, alkyl, haloalkyl, alkenyl, cycloalkyl, heterocycle, aryl, heteroaryl, cyano, nitro, —C(O)R¹⁹, —OC(O)R¹⁹, —NR¹⁹C(O)R¹⁹, —C(O)OR¹⁹, —OC(O)OR¹⁹, —NR¹⁹C(O)OR¹⁹, —C(O)N(R¹⁹)₂, —OC(O)N(R¹⁹)₂, —NR¹⁹C(O)N(R¹⁹)₂, —OR¹⁹, —N(R¹⁹)₂, —S(O)R¹⁹, —S(O)₂R¹⁹, —S(O)OR¹⁹, —S(O)₂OR¹⁹, —S(O)N(R¹⁹)₂, S(O)₂N(R¹⁹)₂, ═O, and —SR¹⁹;

R¹⁹ is independently selected at each instance from hydrogen, alkyl, haloalkyl, cycloalkyl, aryl, heterocycle, and heteroaryl; and

CDK2 Recognition Moiety is a molecule which can bind to or otherwise anchors too CDK2.

56. The compound of embodiment 1, wherein the compound is Formula:

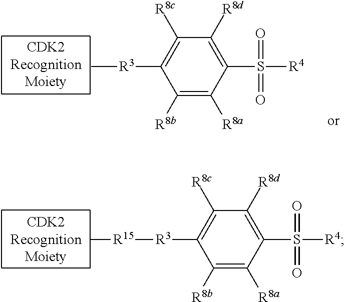

or a pharmaceutically acceptable salt thereof.

57. The compound of any one of embodiments 53-56, wherein R³ is bond.

58. The compound of any one of embodiments 53-56, wherein R³ is phenyl optionally substituted as allowed by valence with 1, 2, 3, or 4 substituents selected from R⁷.

59. The compound of any one of embodiments 53-56, wherein R³ is alkyl optionally substituted as allowed by valence with 1, 2, 3, or 4 substituents selected from R⁷.

60. The compound of any one of embodiments 53-56, wherein R³ is alkenyl optionally substituted as allowed by valence with 1, 2, 3, or 4 substituents selected from R⁷.

61. The compound of any one of embodiments 53-56, wherein R³ is haloalkyl optionally substituted as allowed by valence with 1, 2, 3, or 4 substituents selected from R⁷.

62. The compound of any one of embodiments 53-56, wherein R³ is cycloalkyl optionally substituted as allowed by valence with 1, 2, 3, or 4 substituents selected from R⁷.

63. The compound of any one of embodiments 53-56, wherein R³ is aryl optionally substituted as allowed by valence with 1, 2, 3, or 4 substituents selected from R⁷.

64. The compound of any one of embodiments 53-56, wherein R³ is heteroaryl optionally substituted as allowed by valence with 1, 2, 3, or 4 substituents selected from R⁷.

65. The compound of any one of embodiments 53-56, wherein R³ is bicycle optionally substituted as allowed by valence with 1, 2, 3, or 4 substituents selected from R⁷.

66. The compound of any one of embodiments 1-4 or 22-65, wherein R¹ and R⁴ are

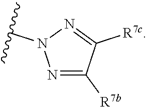

67. The compound of any one of embodiments 1-4 or 22-65, wherein R¹ and R⁴ are

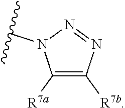

68. The compound of any one of embodiments 1-4 or 22-65, wherein R¹ and R⁴ are

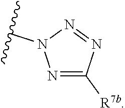

69. The compound of any one of embodiments 1-4 or 22-65, wherein R¹ and R⁴ are

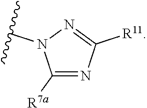

70. The compound of any one of embodiments 1-4 or 22-65, wherein R¹ and R⁴ are

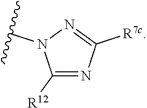

71. The compound of any one of embodiments 1-4 or 22-65, wherein R¹ and R⁴ are

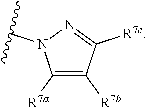

72. The compound of any one of embodiments 1-4 or 22-65, wherein R¹ and R⁴ are

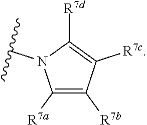

73. The compound of any one of embodiments 1-4 or 22-65, wherein R¹ and R⁴ are

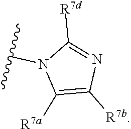

74. The compound of any one of embodiments 1-4 or 22-65, wherein R¹ and R⁴ are a bicyclic heteroaryl which is optionally substituted as allowed by valence with 1, 2, 3, or 4 substituents selected from R⁷.

75. The compound of any one of embodiments 1-4 or 22-65, wherein R⁴ is a heteroaryl group, where the bond to the sulfur atom is through the nitrogen present in the cycle, and each heteroaryl is optionally substituted as allowed by valence with 1, 2, 3, or 4 substituents selected from R⁷.

76. The compound of any one of embodiments 1-75, wherein R⁶ is independently selected at each instance from alkyl, haloalkyl, cycloalkyl, aryl, heterocycle, and heteroaryl; each of which is optionally substituted as allowed by valence with 1, 2, 3, or 4 substituents selected from R¹⁸.

77. The compound of any one of embodiments 1-75, wherein R⁶ is alkyl optionally substituted as allowed by valence with 1, 2, 3, or 4 substituents selected from R¹⁸.

78. The compound of any one of embodiments 1-75, wherein R⁶ is haloalkyl optionally substituted as allowed by valence with 1, 2, 3, or 4 substituents selected from R¹⁸.

79. The compound of any one of embodiments 1-75, wherein R⁶ is cycloalkyl optionally substituted as allowed by valence with 1, 2, 3, or 4 substituents selected from R¹⁸.

80. The compound of any one of embodiments 1-75, wherein R⁶ is aryl optionally substituted as allowed by valence with 1, 2, 3, or 4 substituents selected from R¹⁸.

81. The compound of any one of embodiments 1-75, wherein R⁶ is heterocycle optionally substituted as allowed by valence with 1, 2, 3, or 4 substituents selected from R¹⁸.

82. The compound of any one of embodiments 1-75, wherein R⁶ is heteroaryl optionally substituted as allowed by valence with 1, 2, 3, or 4 substituents selected from R¹⁸.

83. The compound of any one of embodiments 76-82, wherein R⁶ is not substituted with R¹⁸.

84. The compound of any one of embodiments 76-82, wherein R⁶ is substituted with 1 substituent selected from R¹⁸.

85. The compound of any one of embodiments 76-82, wherein R⁶ is substituted with 2 substituents independently selected from R¹⁸.

86. The compound of any one of embodiments 76-82, wherein R⁶ is substituted with 3 substituents independently selected from R¹⁸.

87. The compound of any one of embodiments 1-86, wherein R⁷, R^{7a}, R^{7b}, R^{7c}, and R^{7d} are independently selected at each instance from hydrogen, halogen, alkyl, haloalkyl, alkenyl, cycloalkyl, heterocycle, aryl, heteroaryl, cyano, and nitro, wherein each alkyl, haloalkyl, alkenyl, cycloalkyl, heterocycle, aryl, and heteroaryl is optionally substituted as allowed by valence with 1, 2, 3, or 4 substituents selected from R¹⁷.

88. The compound of any one of embodiments 1-87, wherein one of R⁷, R^{7a}, R^{7b}, R^{7c}, and R^{7d} is alkyl optionally substituted as allowed by valence with 1, 2, 3, or 4 substituents selected from R¹⁷.

89. The compound of any one of embodiments 1-87, wherein one of R⁷, R^{7a}, R^{7b}, R^{7c}, and R^{7d} is haloalkyl optionally substituted as allowed by valence with 1, 2, 3, or 4 substituents selected from R¹⁷.

90. The compound of any one of embodiments 1-87, wherein one of R⁷, R^{7a}, R^{7b}, R^{7c}, and R^{7d} is phenyl optionally substituted as allowed by valence with 1, 2, 3, or 4 substituents selected from R¹⁷.

91. The compound of any one of embodiments 1-87, wherein one of R⁷, R^{7a}, R^{7b}, R^{7c}, and R^{7d} is heteroaryl optionally substituted as allowed by valence with 1, 2, 3, or 4 substituents selected from R¹⁷.

92. The compound of any one of embodiments 1-87, wherein one of R⁷, R^{7a}, R^{7b}, R^{7c}, and R^{7d} is haloalkyl optionally substituted as allowed by valence with 1, 2, 3, or 4 substituents selected from R¹⁷.

93. The compound of any one of embodiments 1-87, wherein one of R⁷, R^{7a}, R^{7b}, R^{7c}, and R^{7d} is heterocycle optionally substituted as allowed by valence with 1, 2, 3, or 4 substituents selected from R¹⁷.

94. The compound of any one of embodiments 1-87, wherein one of R⁷, R^{7a}, R^{7b}, R^{7c}, and R^{7d} is cycloalkyl optionally substituted as allowed by valence with 1, 2, 3, or 4 substituents selected from R¹⁷.

95. The compound of any one of embodiments 87-94, wherein R⁷, R^{7a}, R^{7b}, R^{7c}, and R^{7d} are not substituted.

96. The compound of any one of embodiments 87-94, wherein R⁷, R^{7a}, R^{7b}, R^{7c}, and R^{7d} are optionally substituted with 1 or 2 substituents selected from R¹⁷.

97. The compound of any one of embodiments 1-87, wherein one of R⁷, R^{7a}, R^{7b}, R^{7c}, and R^{7d} is halogen.

98. The compound of any one of embodiments 1-87, wherein one of R⁷, R^{7a}, R^{7b}, R^{7c}, and R^{7d} is —OR⁶.

99. The compound of any one of embodiments 1-87, wherein one of R⁷, R^{7a}, R^{7b}, R^{7c}, and R^{7d} is ═O.

100. The compound of any one of embodiments 1-87, wherein one of R⁷, R^{7a}, R^{7b}, R^{7c}, and R^{7d} is cyano.

101. The compound of any one of embodiments 1-87, wherein one of R⁷, R^{7a}, R^{7b}, R^{7c}, and R^{7d} is nitro.

102. The compound of any one of embodiments 1-101, wherein R¹⁷ is selected in each instance from halogen, alkyl, haloalkyl, alkenyl, and cyano.

103. A compound selected from Table 1.

104. A compound selected from Table 2.

105. A pharmaceutical composition comprising a compound of any one of embodiments 1-104 or a pharmaceutically acceptable salt thereof and a pharmaceutically acceptable excipient.

106. The pharmaceutical composition of embodiment 105, wherein the composition is suitable for oral delivery.

107. The pharmaceutical composition of embodiment 105, wherein the composition is suitable for intravenous delivery.

108. The pharmaceutical composition of embodiment 105, wherein the composition is suitable for parental delivery.

109. A method of treating a disorder mediated by CDK2 comprising administering an effective amount of a compound of any one of embodiments 1-104 or a pharmaceutically acceptable salt thereof or a pharmaceutical composition of any one of embodiments 105-108 to a patient in need thereof.

110. The method of embodiment 109, wherein the patient is a human.

111. The method of embodiment 109 or 110, wherein the disorder is a cancer.

112. The method of embodiment 111, wherein the cancer is a solid cancer.

113. The method of embodiment 111, wherein the cancer is a hematological cancer.

114. The method of embodiment 111, wherein the disorder is breast cancer.

115. The method of embodiment 114, wherein the breast cancer is triple negative breast cancer.

116. The method of embodiment 111, wherein the disorder is hormone receptor positive HER2 negative breast cancer.

117. The method of embodiment 111, wherein the disorder is ovarian cancer.

118. The method of embodiment 111, wherein the disorder is fallopian tube cancer.

119. The method of embodiment 111, wherein the disorder is peritoneal cancer.

120. The method of any one of embodiments 111-119, wherein the cancer is metastatic.

121. The method of any one of embodiments 111-120, wherein the cancer is relapsed.

122. The method of any one of embodiments 111-121, wherein the cancer is refractory.

123. The method of embodiment 109 or 110, wherein the disorder is a tumor.

124. Use of a compound of any one of embodiments 1-104 or a pharmaceutically acceptable salt thereof or a pharmaceutical composition of any one of embodiments 105-108 to treat a CDK2 mediated disorder in a patient in need thereof.

125. Use of a compound of any one of embodiments 1-104 or a pharmaceutically acceptable salt thereof or a pharmaceutical composition of any one of embodiments 105-108 in the manufacture of a medicament to treat a CDK2 mediated disorder in a patient in need thereof.

126. The use of embodiment 124 or 125, wherein the patient is a human.

127. The use of any one of embodiments 124-126, wherein the disorder is a cancer.

128. The use of embodiment 127, wherein the cancer is a solid cancer.

129. The use of embodiment 127, wherein the cancer is a hematological cancer.

130. The use of embodiment 127, wherein the disorder is breast cancer.

131. The use of embodiment 130, wherein the breast cancer is triple negative breast cancer.

132. The use of embodiment 127, wherein the disorder is hormone receptor positive HER2 negative breast cancer.

133. The use of embodiment 127, wherein the disorder is ovarian cancer.

134. The use of embodiment 127, wherein the disorder is fallopian tube cancer.

135. The use of embodiment 127, wherein the disorder is peritoneal cancer.

136. The use of any one of embodiments 127-135, wherein the cancer is metastatic.

137. The use of any one of embodiments 127-136, wherein the cancer is relapsed.

138. The use of any one of embodiments 127-137, wherein the cancer is refractory.

139. The use of any one of embodiments 124-126, wherein the disorder is a tumor.

140. A compound of any one of embodiments 1-104 or a pharmaceutically acceptable salt thereof or a pharmaceutical composition of any one of embodiments 105-108 for use in the treatment of a CDK2 mediated disorder in a patient in need thereof.

141. The compound or pharmaceutical composition of embodiment 140, wherein the patient is a human.

142. The compound or pharmaceutical composition of embodiment 140 or 141, wherein the disorder is a cancer.

143. The compound or pharmaceutical composition of embodiment 142, wherein the cancer is a solid cancer.

144. The compound or pharmaceutical composition of embodiment 142, wherein the cancer is a hematological cancer.

145. The compound or pharmaceutical composition of embodiment 142, wherein the disorder is breast cancer.

146. The compound or pharmaceutical composition of embodiment 145, wherein the breast cancer is triple negative breast cancer.

147. The compound or pharmaceutical composition of embodiment 142, wherein the disorder is hormone receptor positive HER2 negative breast cancer.

148. The compound or pharmaceutical composition of embodiment 142, wherein the disorder is ovarian cancer.

149. The compound or pharmaceutical composition of embodiment 142, wherein the disorder is fallopian tube cancer.

150. The compound or pharmaceutical composition of embodiment 142, wherein the disorder is peritoneal cancer.

151. The compound or pharmaceutical composition of any one of embodiments 142-150, wherein the cancer is metastatic.

152. The compound or pharmaceutical composition of any one of embodiments 142-151, wherein the cancer is relapsed.

153. The compound or pharmaceutical composition of any one of embodiments 142-152, wherein the cancer is refractory.

154. The compound or pharmaceutical composition of embodiment 140 or 141, wherein the disorder is a tumor.

### Additional Heteroaryl Sulfonyl Compounds of the Present Invention

In certain embodiments the heteroaryl sulfonyl compound of the present invention is

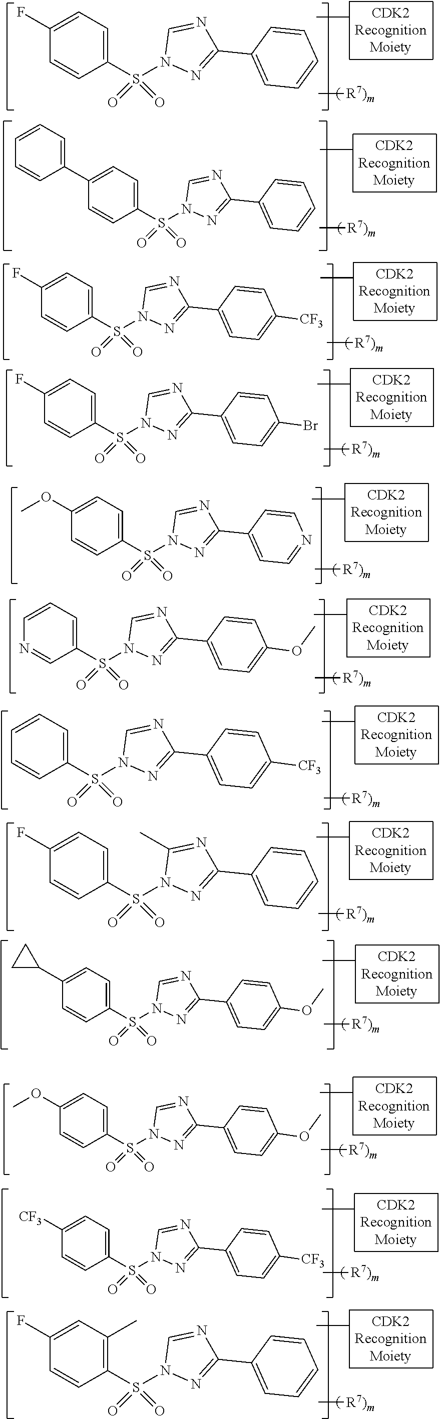

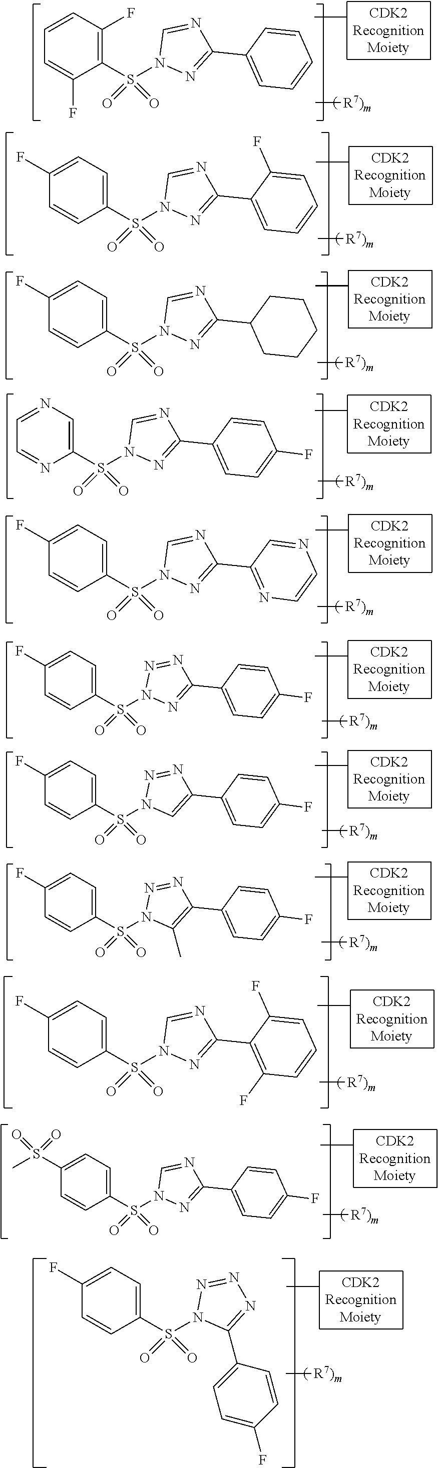

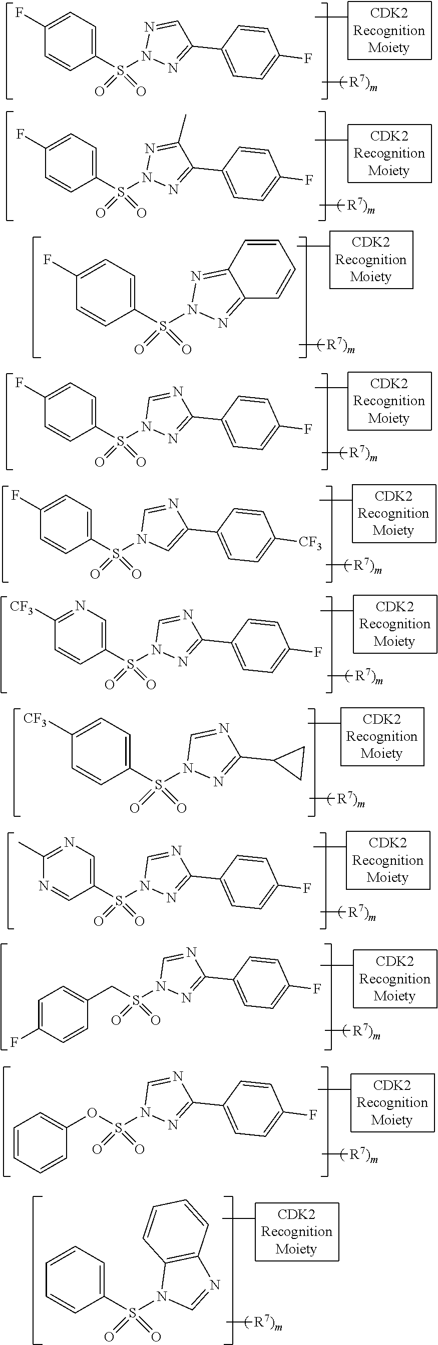

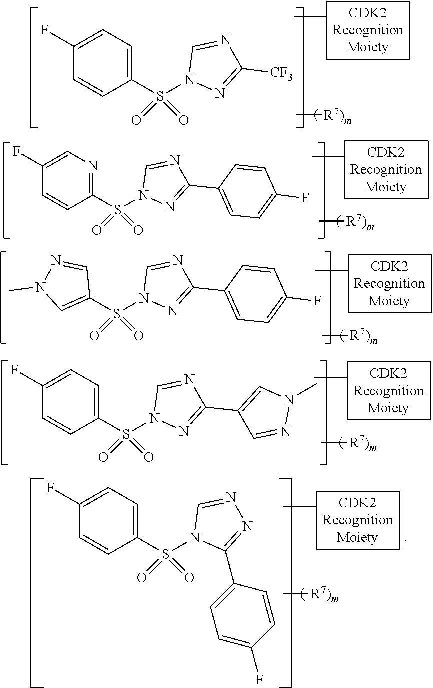

In certain embodiments the heteroaryl sulfonyl compound of the present invention is selected from:

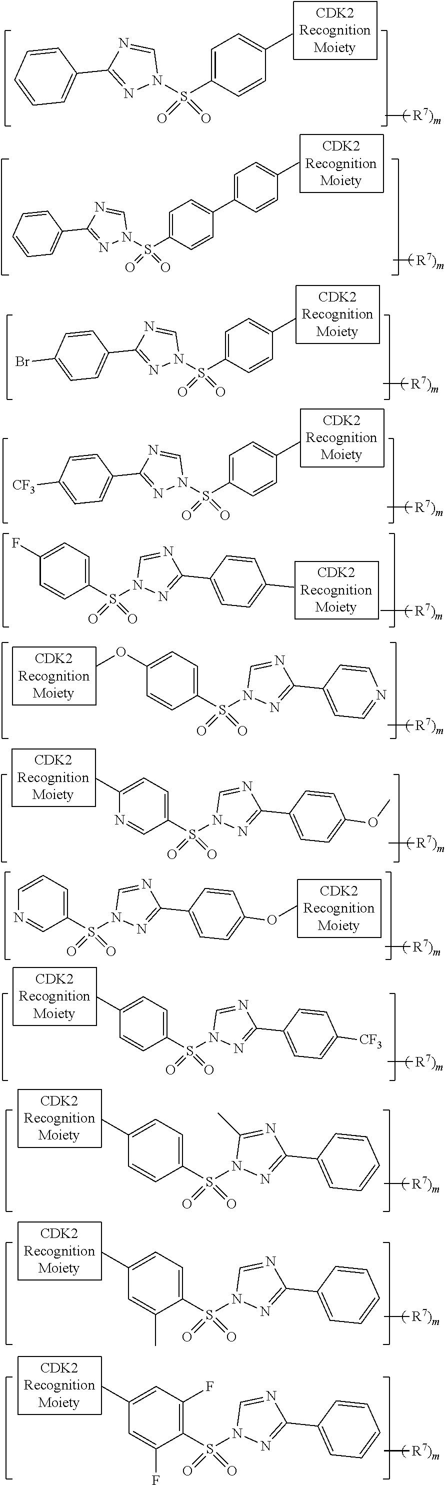

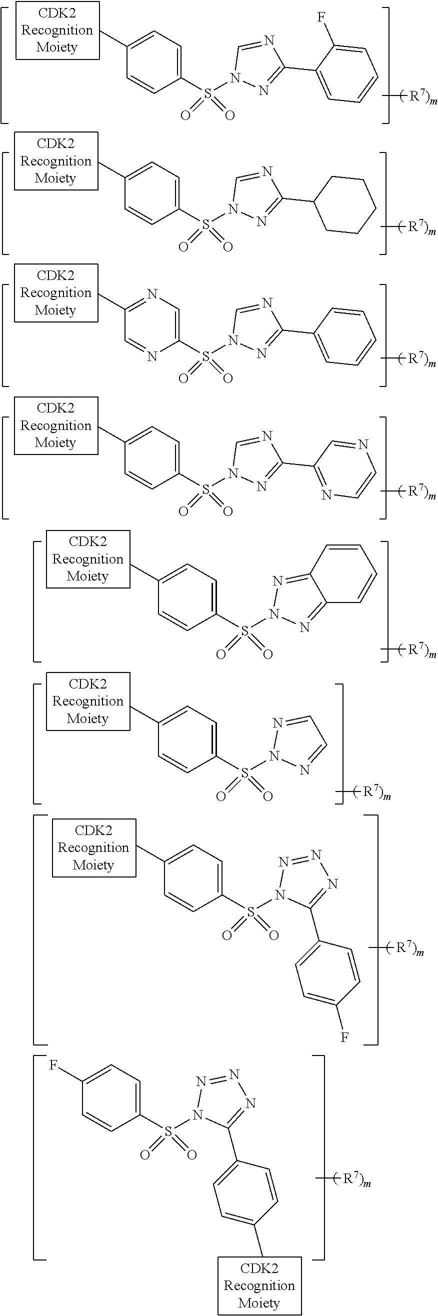

Non-limiting examples of heteroaryl sulfonyl compounds of the present invention include:

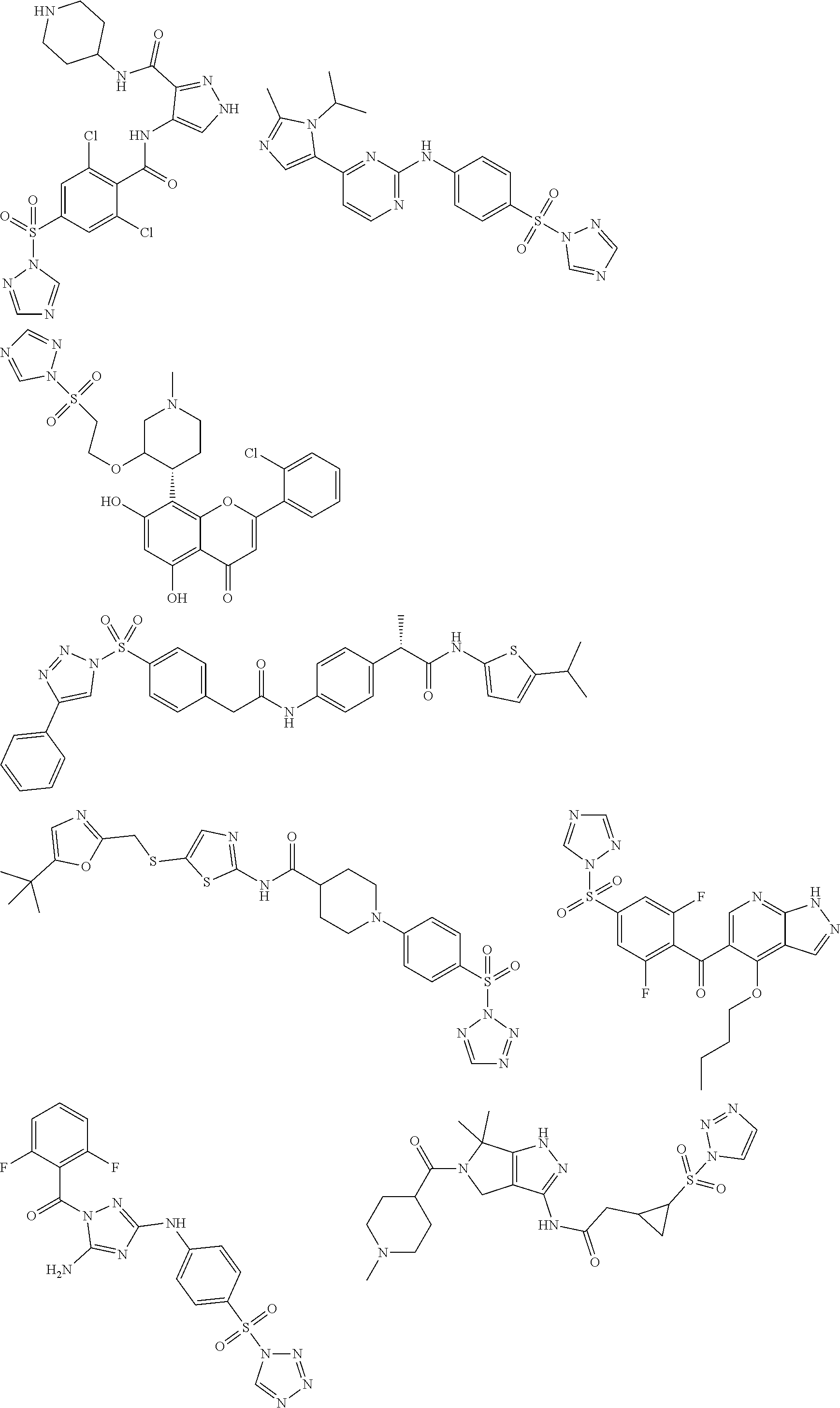

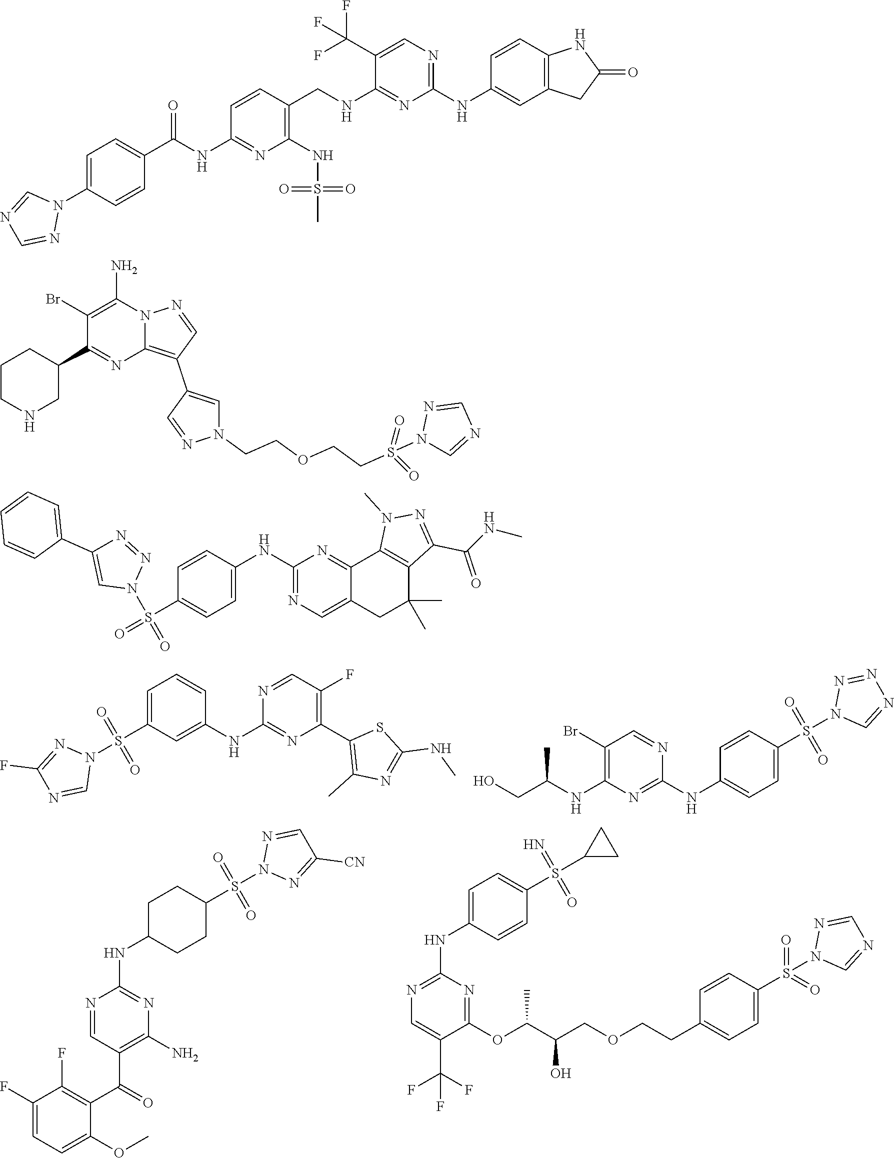

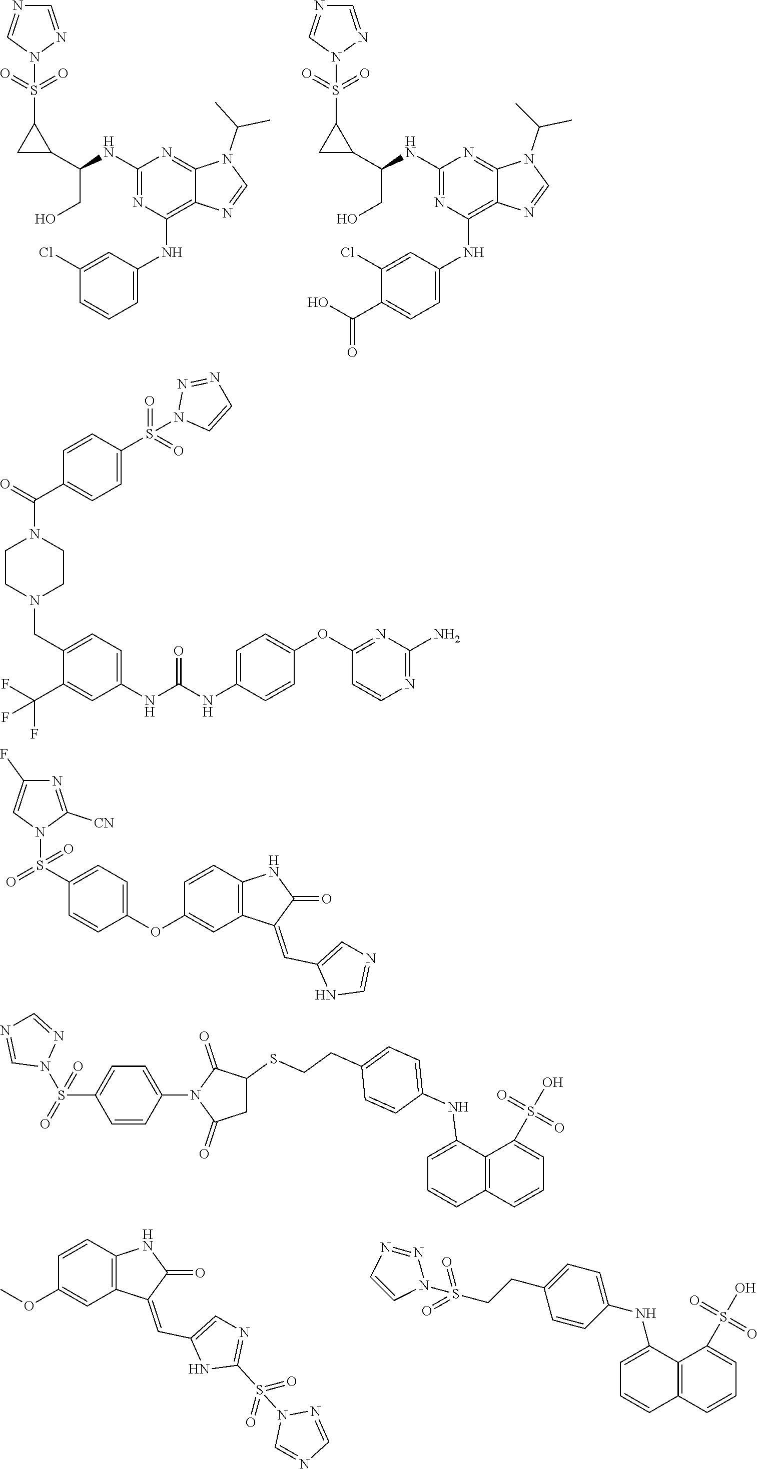

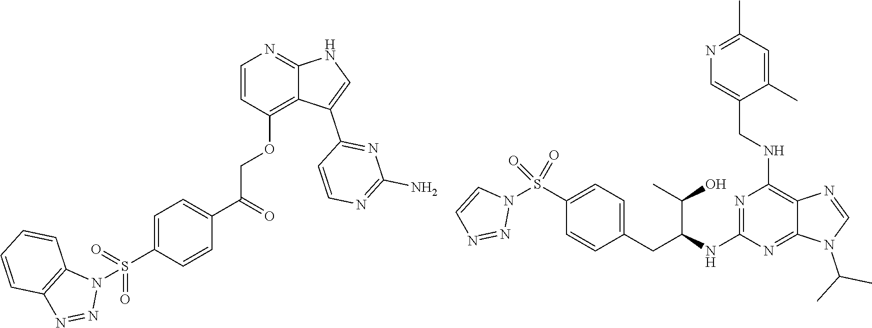 [SMILES: Nc1nccc(-c2c[nH]c3nccc(OCC(=O)c4ccc(S(=O)(=O)n5nnc6ccccc65)cc4)c23)n1 ; Cc1cc(C)c(CNc2nc(N[C@@H](Cc3ccc(S(=O)(=O)n4ccnn4)cc3)[C@@H](C)O)nc3c2ncn3C(C)C)cn1]

In certain embodiments, the heteroaryl sulfonyl compound of the present invention is selected from:

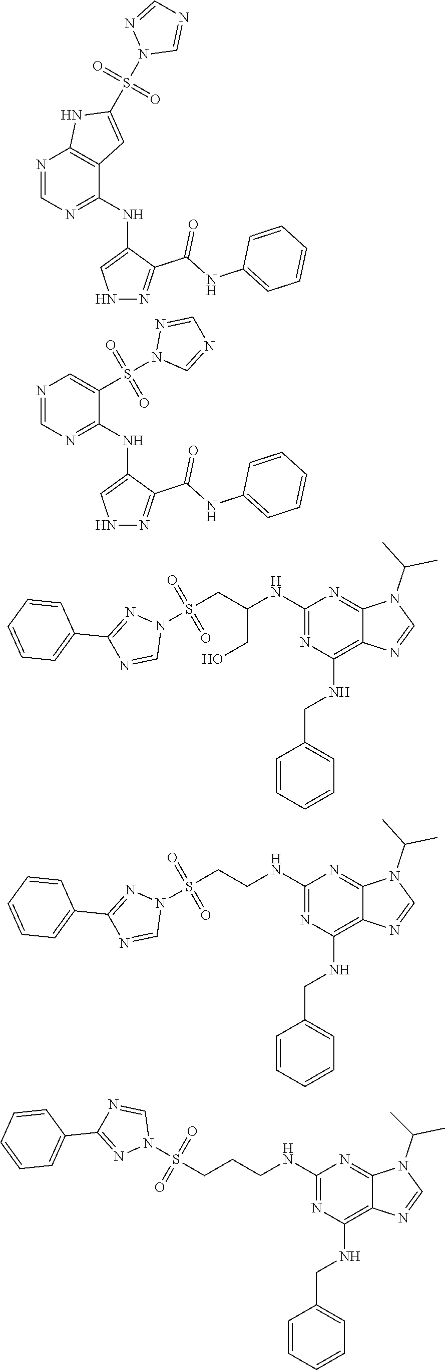

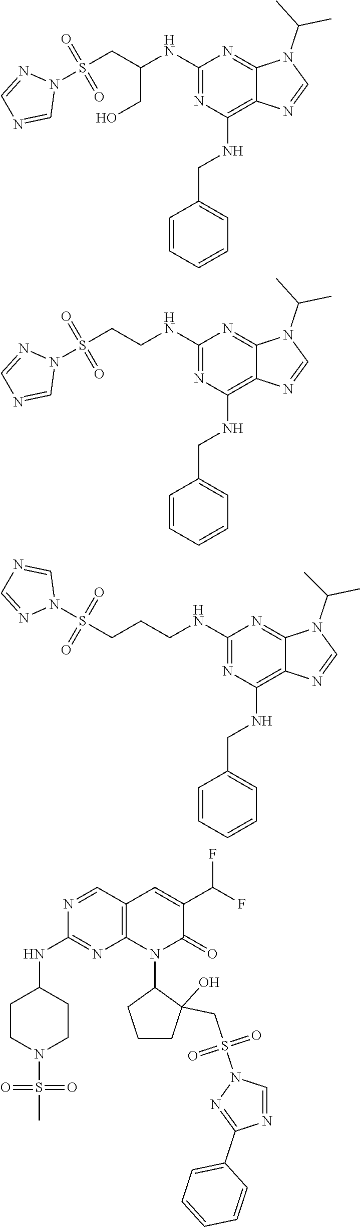

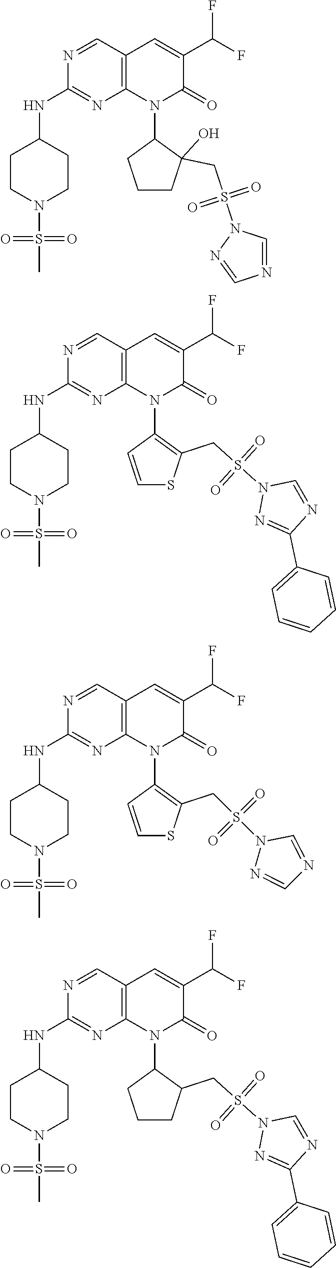

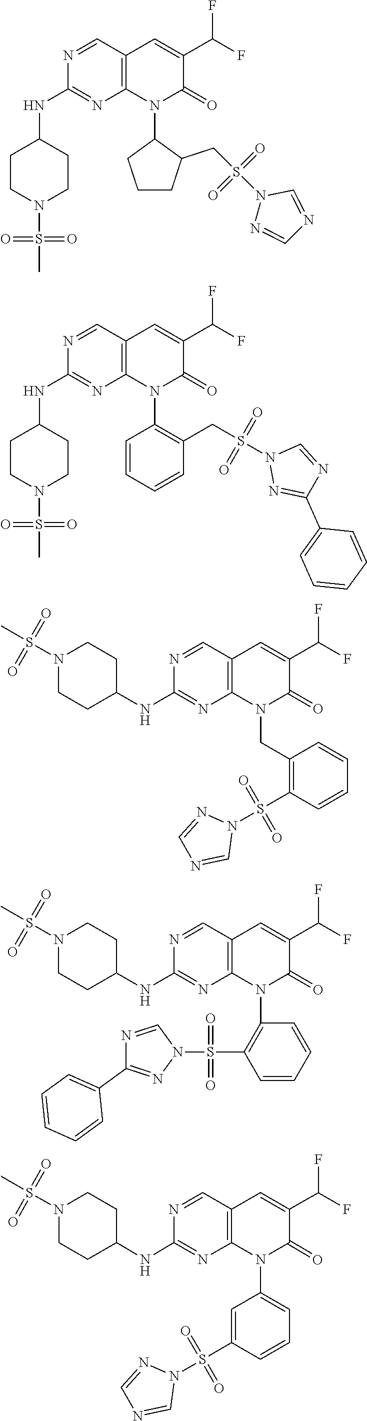

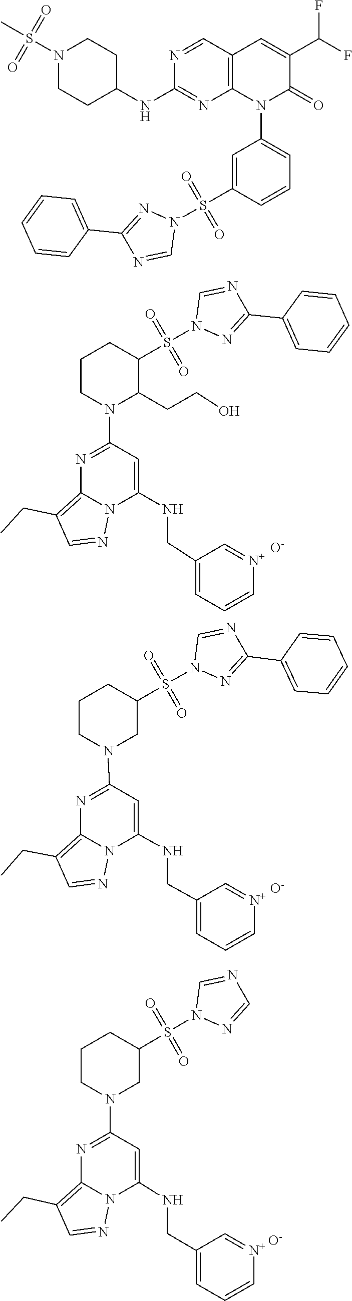

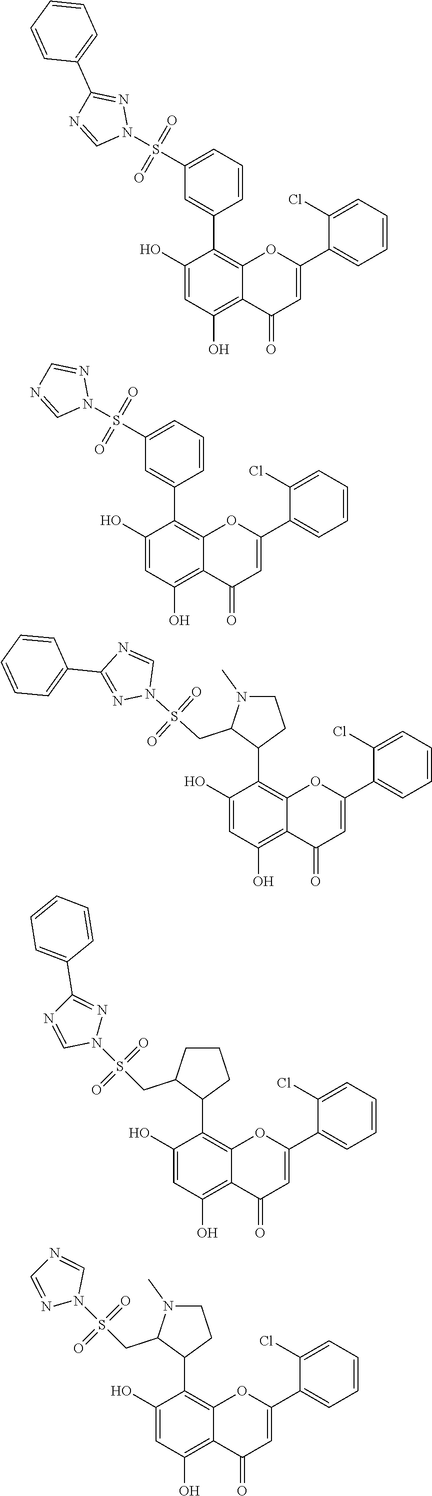

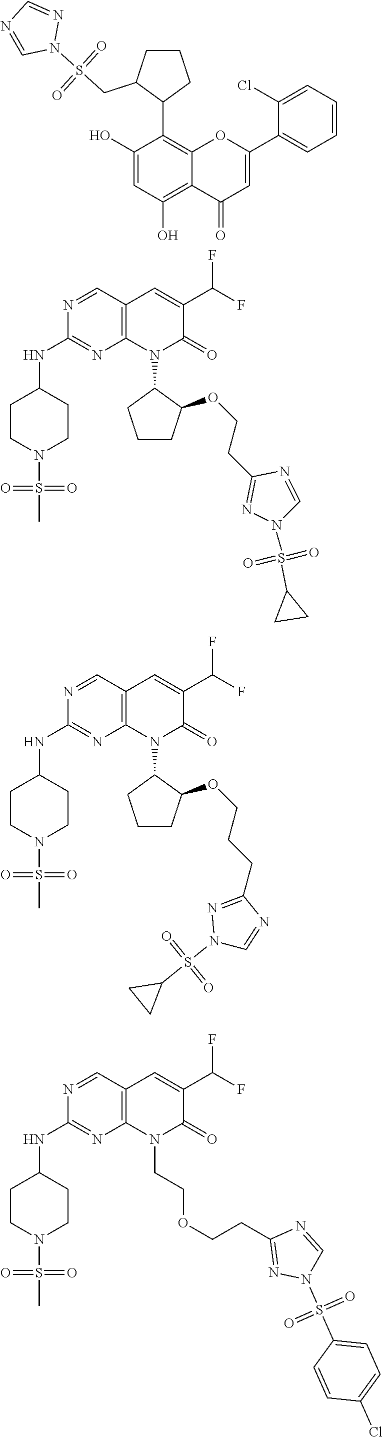

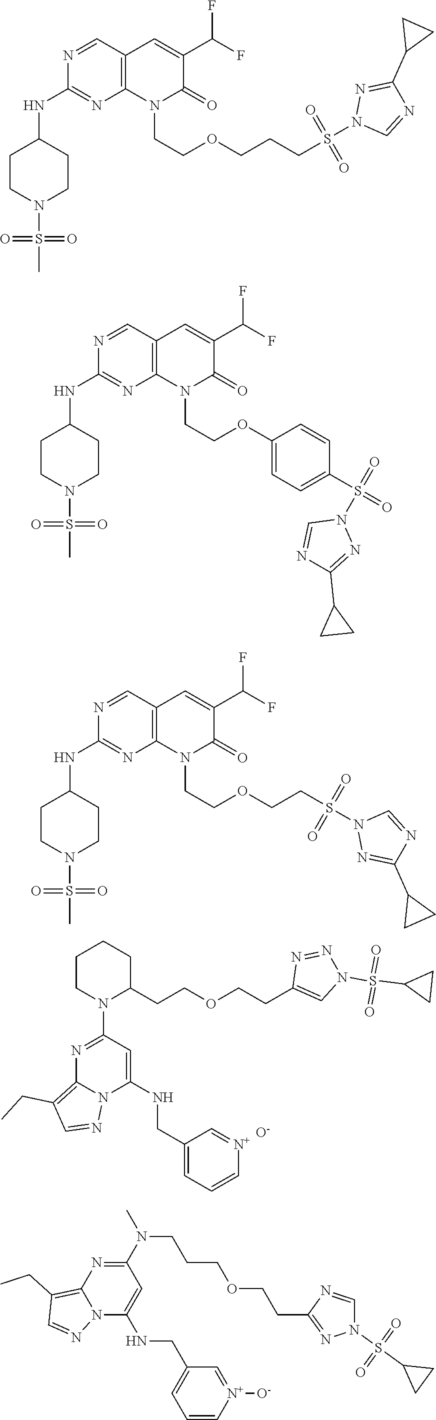

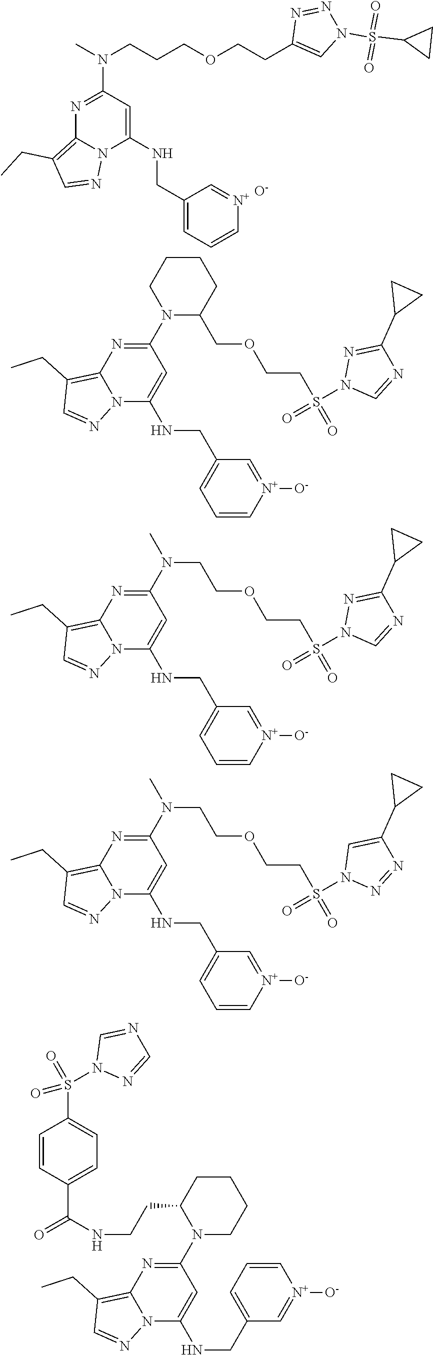

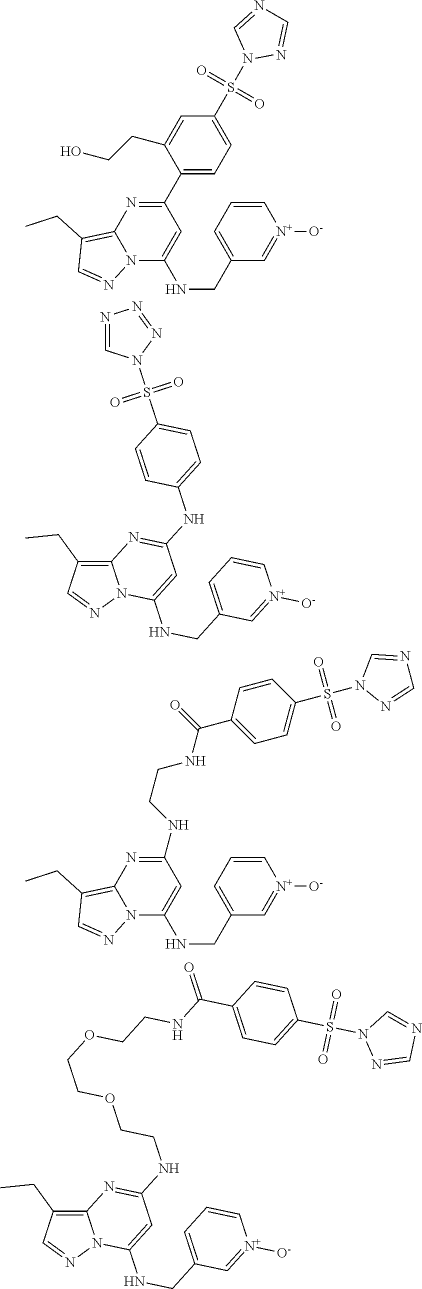

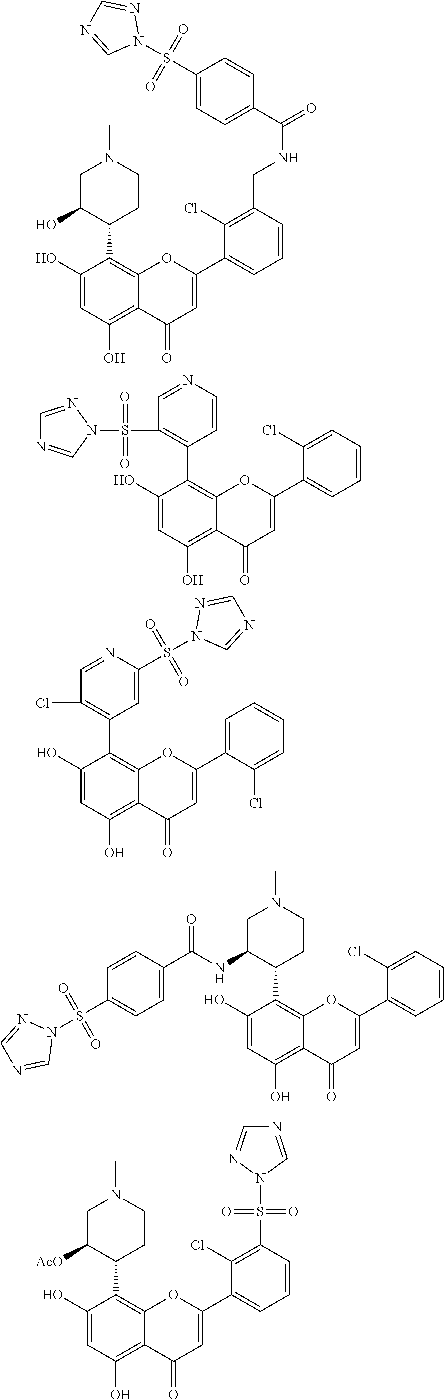

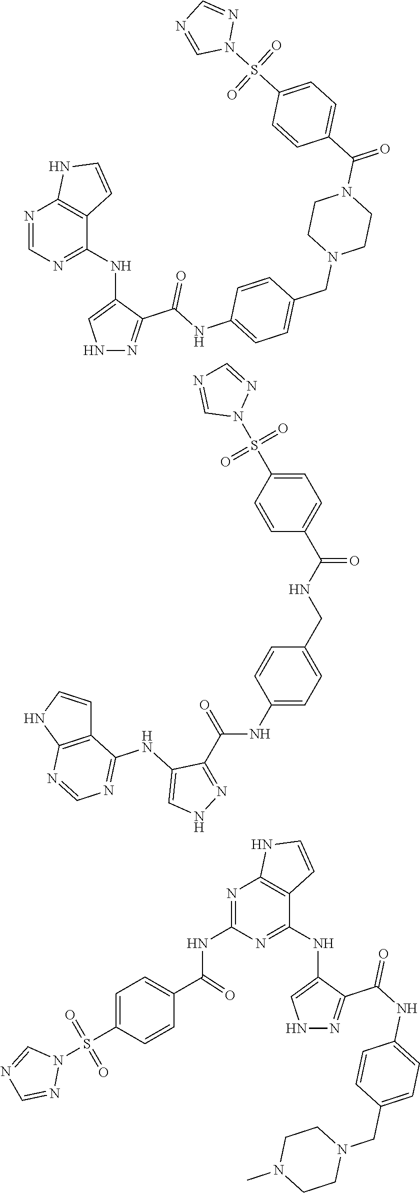

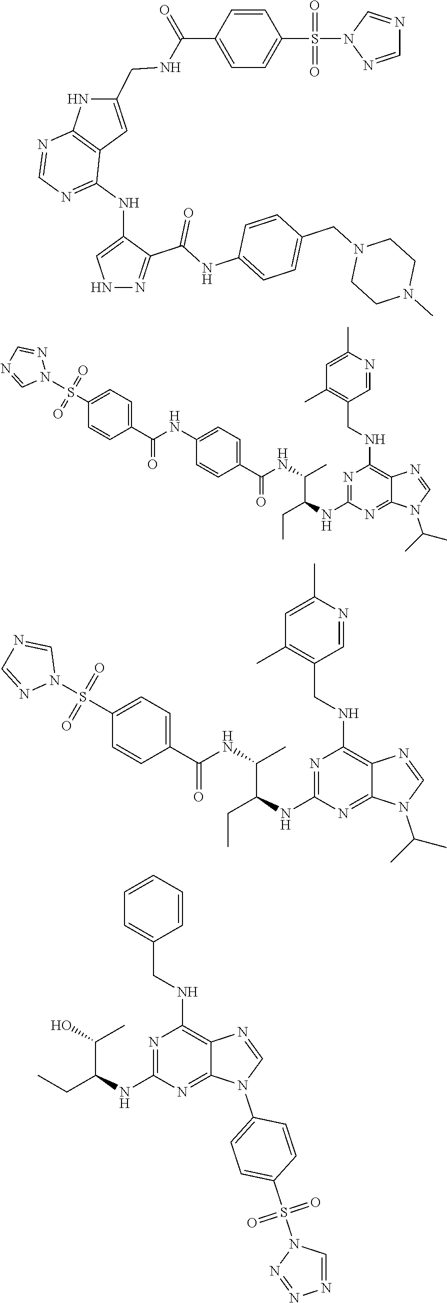

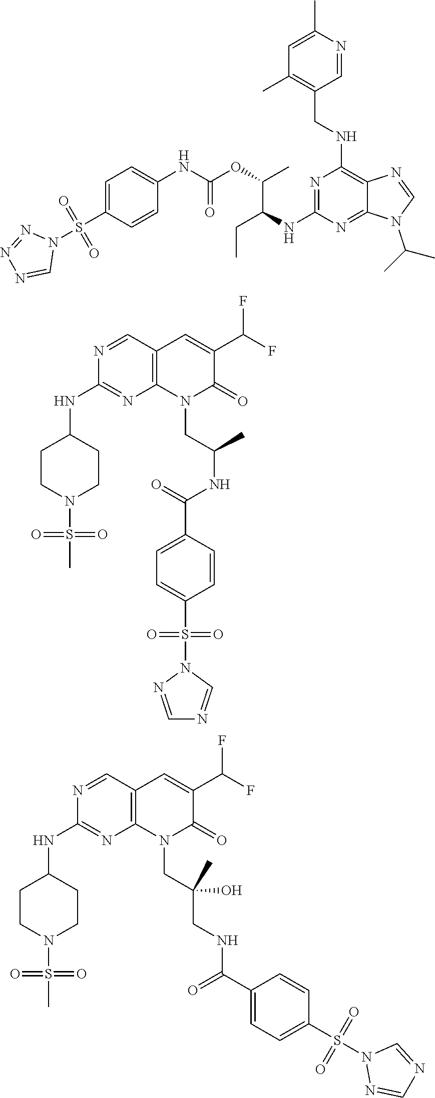

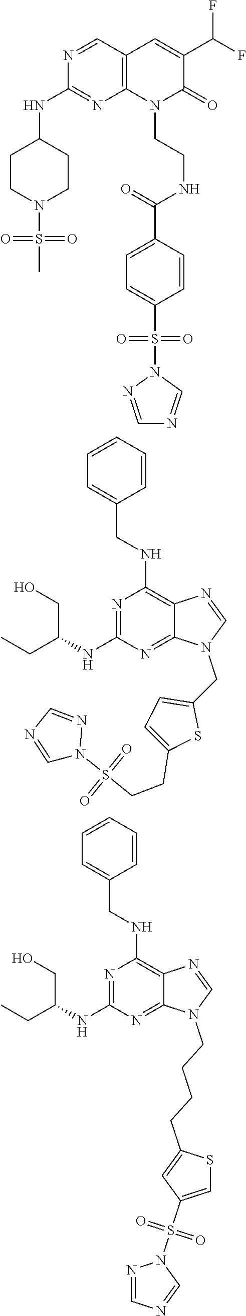

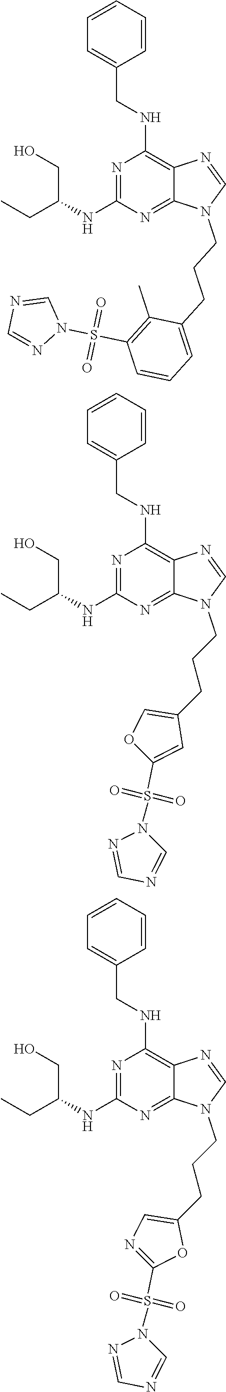

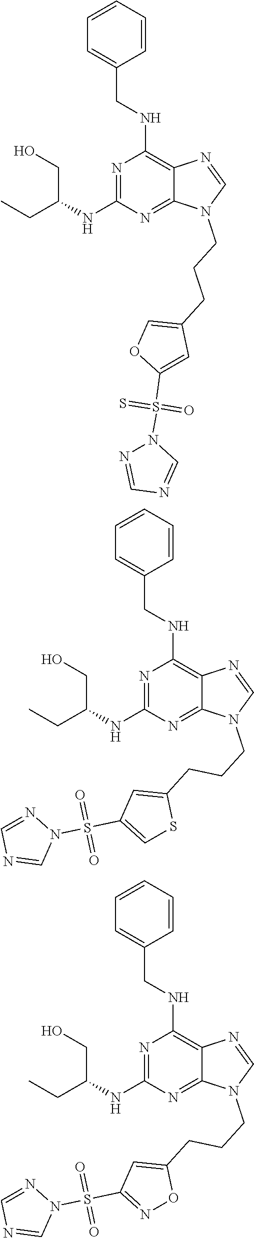

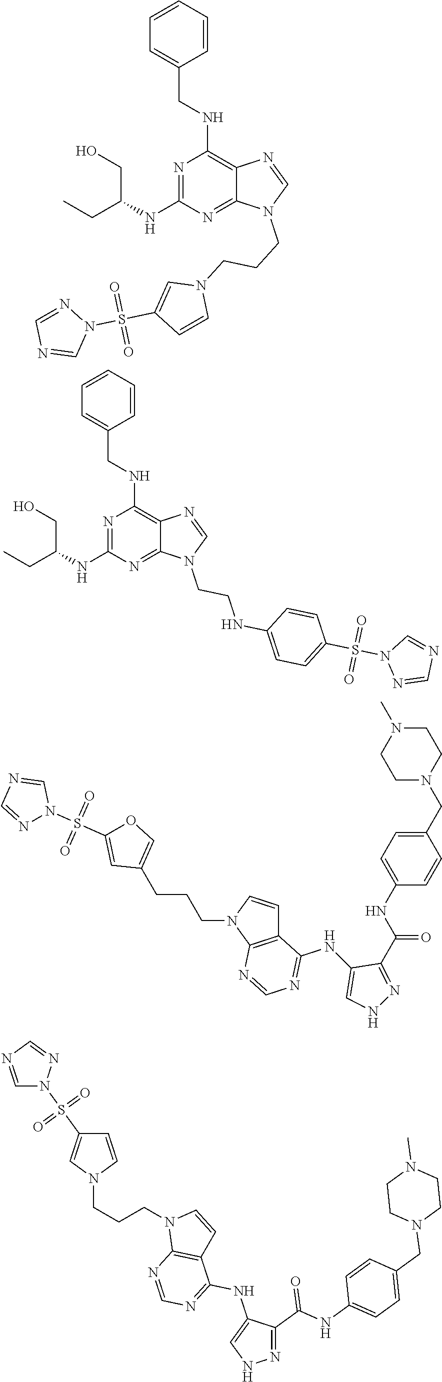

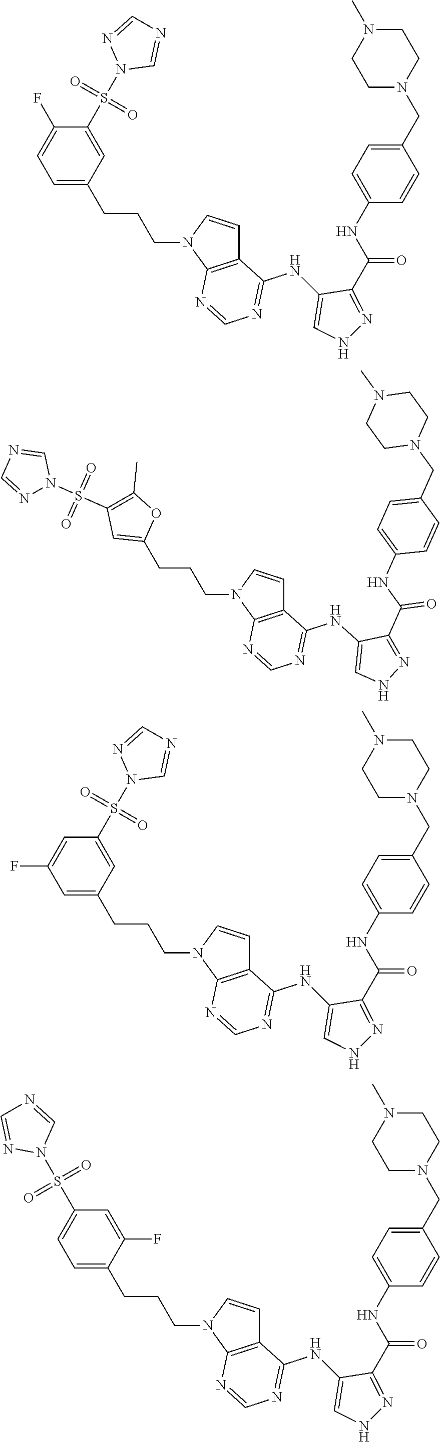

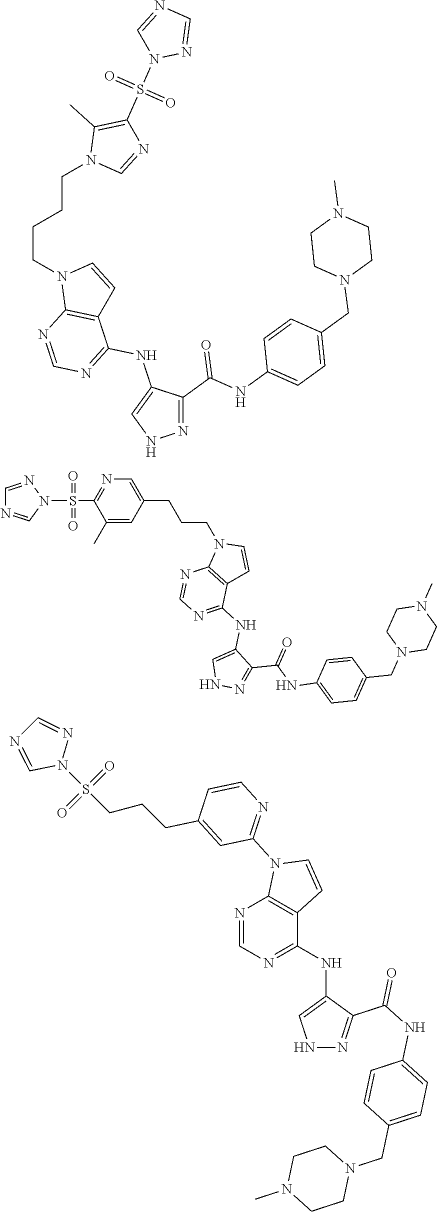

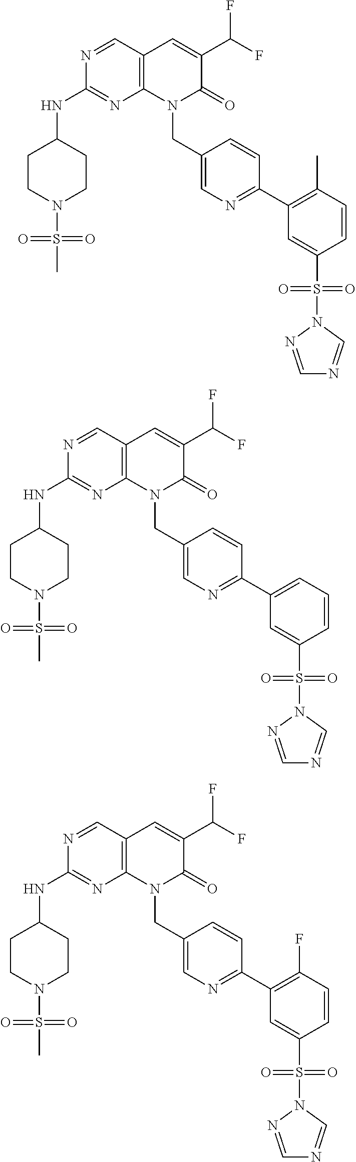 [SMILES: Cc1ccc(S(=O)(=O)n2cncn2)cc1-c1ccc(Cn2c(=O)c(C(F)F)cc3cnc(NC4CCN(S(C)(=O)=O)CC4)nc32)cn1 ; CS(=O)(=O)N1CCC(Nc2ncc3cc(C(F)F)c(=O)n(Cc4ccc(-c5cccc(S(=O)(=O)n6cncn6)c5)nc4)c3n2)CC1 ; CS(=O)(=O)N1CCC(Nc2ncc3cc(C(F)F)c(=O)n(Cc4ccc(-c5cc(S(=O)(=O)n6cncn6)ccc5F)nc4)c3n2)CC1]

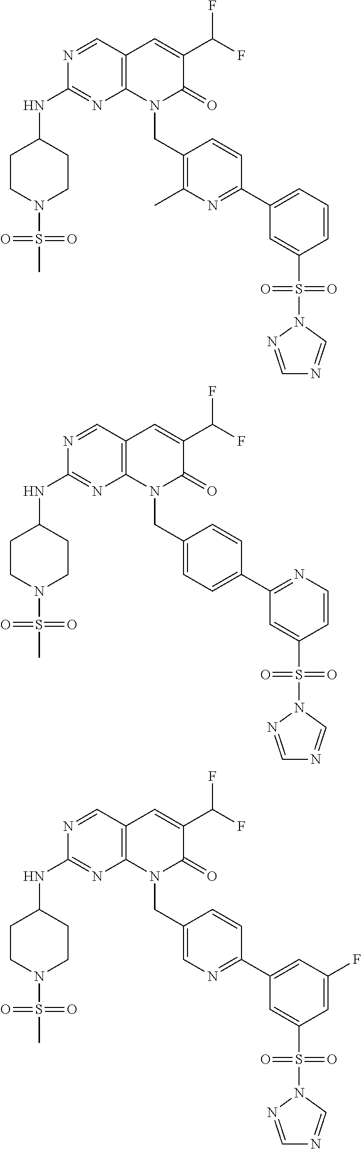 [SMILES: Cc1nc(-c2cccc(S(=O)(=O)n3cncn3)c2)ccc1Cn1c(=O)c(C(F)F)cc2cnc(NC3CCN(S(C)(=O)=O)CC3)nc21 ; CS(=O)(=O)N1CCC(Nc2ncc3cc(C(F)F)c(=O)n(Cc4ccc(-c5cc(S(=O)(=O)n6cncn6)ccn5)cc4)c3n2)CC1 ; CS(=O)(=O)N1CCC(Nc2ncc3cc(C(F)F)c(=O)n(Cc4ccc(-c5cc(F)cc(S(=O)(=O)n6cncn6)c5)nc4)c3n2)CC1]

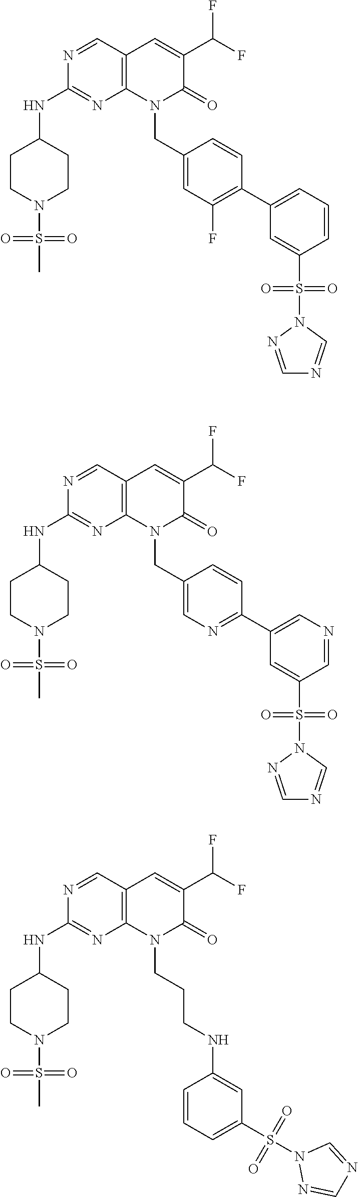 [SMILES: CS(=O)(=O)N1CCC(Nc2ncc3cc(C(F)F)c(=O)n(Cc4ccc(-c5cccc(S(=O)(=O)n6cncn6)c5)c(F)c4)c3n2)CC1 ; CS(=O)(=O)N1CCC(Nc2ncc3cc(C(F)F)c(=O)n(Cc4ccc(-c5cncc(S(=O)(=O)n6cncn6)c5)nc4)c3n2)CC1 ; CS(=O)(=O)N1CCC(Nc2ncc3cc(C(F)F)c(=O)n(CCCNc4cccc(S(=O)(=O)n5cncn5)c4)c3n2)CC1]

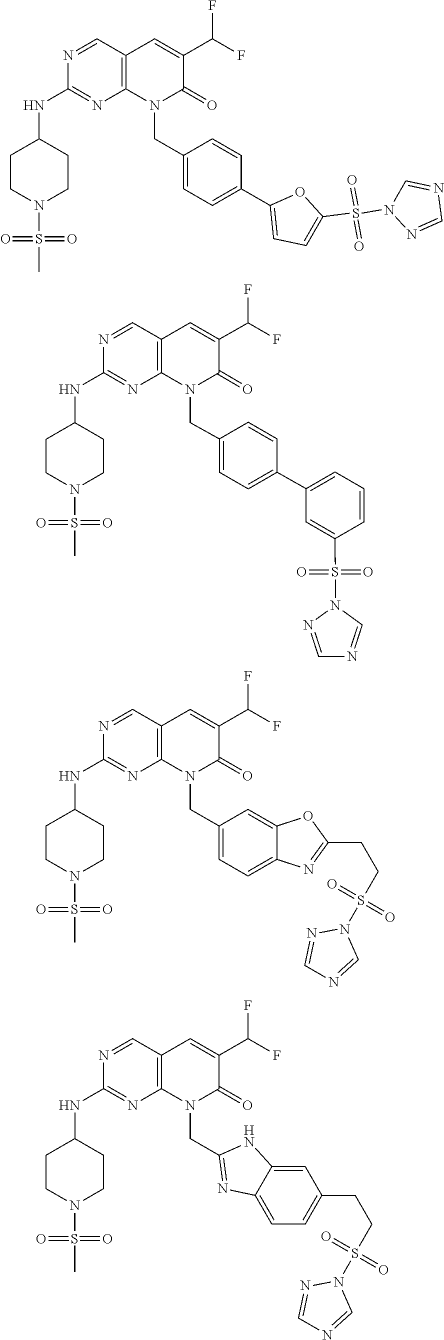 [SMILES: CS(=O)(=O)N1CCC(Nc2ncc3cc(C(F)F)c(=O)n(Cc4ccc(-c5ccc(S(=O)(=O)n6cncn6)o5)cc4)c3n2)CC1 ; CS(=O)(=O)N1CCC(Nc2ncc3cc(C(F)F)c(=O)n(Cc4ccc(-c5cccc(S(=O)(=O)n6cncn6)c5)cc4)c3n2)CC1 ; CS(=O)(=O)N1CCC(Nc2ncc3cc(C(F)F)c(=O)n(Cc4ccc5nc(CCS(=O)(=O)n6cncn6)oc5c4)c3n2)CC1 ; CS(=O)(=O)N1CCC(Nc2ncc3cc(C(F)F)c(=O)n(Cc4nc5ccc(CCS(=O)(=O)n6cncn6)cc5[nH]4)c3n2)CC1]

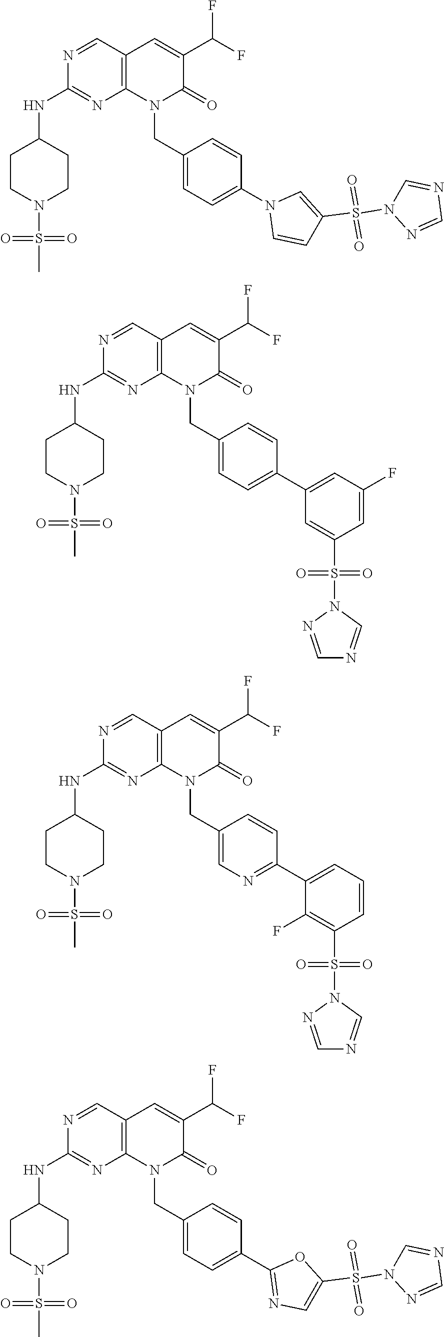 [SMILES: CS(=O)(=O)N1CCC(Nc2ncc3cc(C(F)F)c(=O)n(Cc4ccc(-n5ccc(S(=O)(=O)n6cncn6)c5)cc4)c3n2)CC1 ; CS(=O)(=O)N1CCC(Nc2ncc3cc(C(F)F)c(=O)n(Cc4ccc(-c5cc(F)cc(S(=O)(=O)n6cncn6)c5)cc4)c3n2)CC1 ; CS(=O)(=O)N1CCC(Nc2ncc3cc(C(F)F)c(=O)n(Cc4ccc(-c5cccc(S(=O)(=O)n6cncn6)c5F)nc4)c3n2)CC1 ; CS(=O)(=O)N1CCC(Nc2ncc3cc(C(F)F)c(=O)n(Cc4ccc(-c5ncc(S(=O)(=O)n6cncn6)o5)cc4)c3n2)CC1]

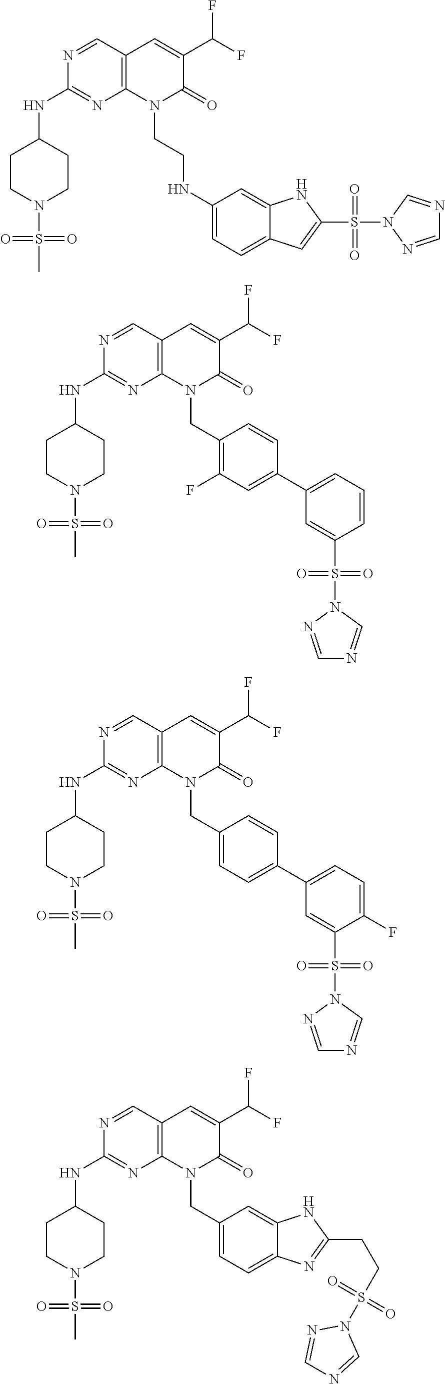

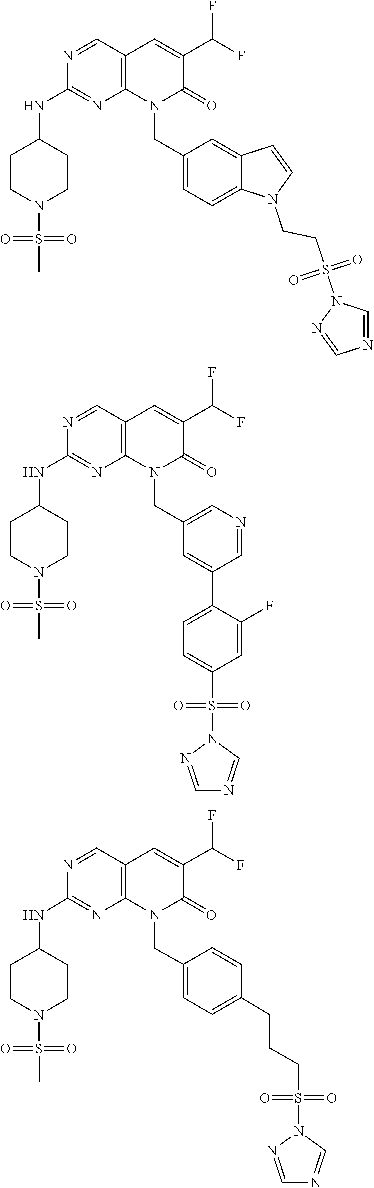

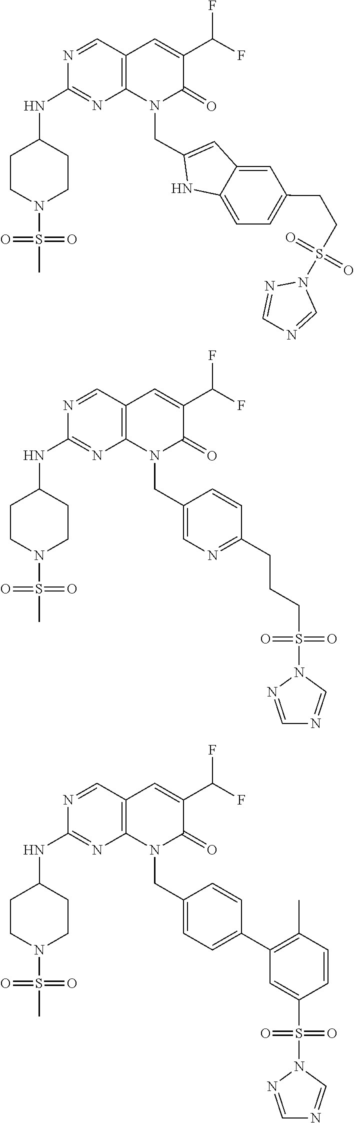 [SMILES: CS(=O)(=O)N1CCC(Nc2ncc3cc(C(F)F)c(=O)n(Cc4cc5cc(CCS(=O)(=O)n6cncn6)ccc5[nH]4)c3n2)CC1 ; CS(=O)(=O)N1CCC(Nc2ncc3cc(C(F)F)c(=O)n(Cc4ccc(CCCS(=O)(=O)n5cncn5)nc4)c3n2)CC1 ; Cc1ccc(S(=O)(=O)n2cncn2)cc1-c1ccc(Cn2c(=O)c(C(F)F)cc3cnc(NC4CCN(S(C)(=O)=O)CC4)nc32)cc1]

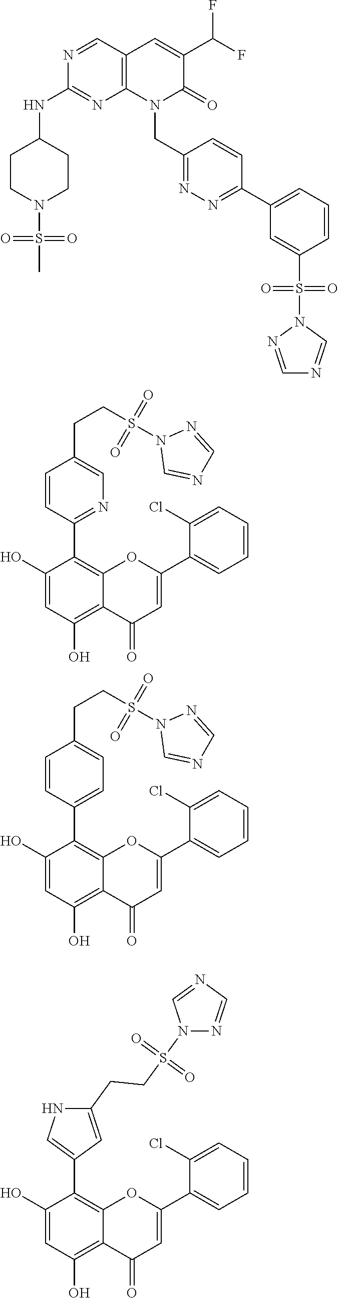

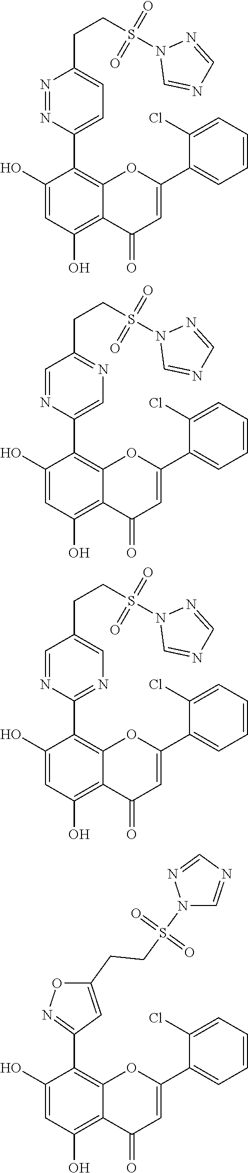

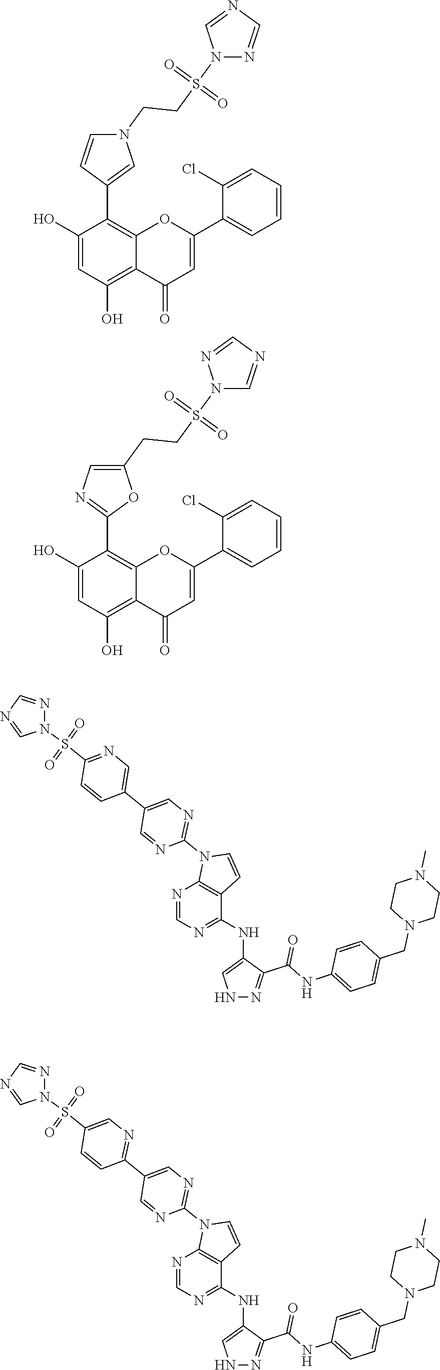 [SMILES: O=c1cc(-c2ccccc2Cl)oc2c(-c3ccn(CCS(=O)(=O)n4cncn4)c3)c(O)cc(O)c12 ; O=c1cc(-c2ccccc2Cl)oc2c(-c3ncc(CCS(=O)(=O)n4cncn4)o3)c(O)cc(O)c12 ; CN1CCN(Cc2ccc(NC(=O)c3n[nH]cc3Nc3ncnc4c3ccn4-c3ncc(-c4ccc(S(=O)(=O)n5cncn5)nc4)cn3)cc2)CC1 ; CN1CCN(Cc2ccc(NC(=O)c3n[nH]cc3Nc3ncnc4c3ccn4-c3ncc(-c4ccc(S(=O)(=O)n5cncn5)cn4)cn3)cc2)CC1]

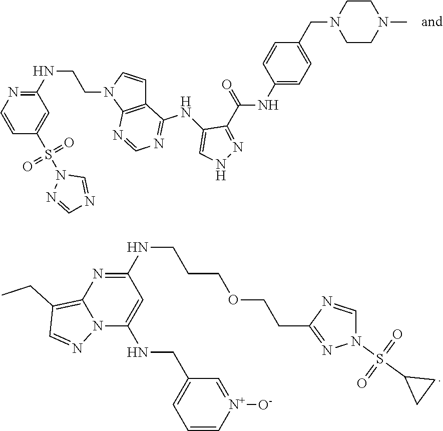

In certain embodiments the heteroaryl sulfonyl compound of the present invention is selected from

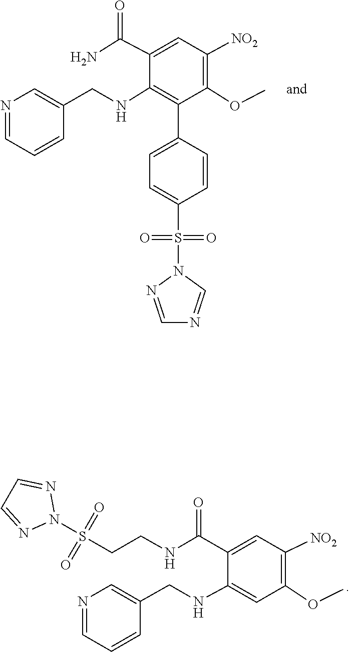 [SMILES: COc1c([N+](=O)[O-])cc(C(N)=O)c(NCc2cccnc2)c1-c1ccc(S(=O)(=O)n2cncn2)cc1 ; COc1cc(NCc2cccnc2)c(C(=O)NCCS(=O)(=O)n2nccn2)cc1[N+](=O)[O-]]

In certain embodiments the heteroaryl sulfonyl compound of the present invention is selected from

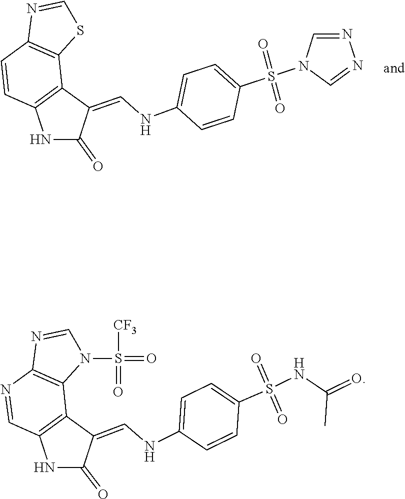 [SMILES: O=C1Nc2ccc3ncsc3c2/C1=C/Nc1ccc(S(=O)(=O)n2cnnc2)cc1 ; CC(=O)NS(=O)(=O)c1ccc(N/C=C2\C(=O)Nc3cnc4ncn(S(=O)(=O)C(F)(F)F)c4c32)cc1]

In certain embodiments the heteroaryl sulfonyl compound of the present invention is selected from

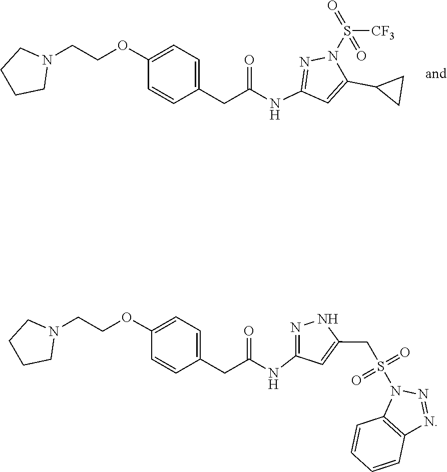 [SMILES: O=C(Cc1ccc(OCCN2CCCC2)cc1)Nc1cc(C2CC2)n(S(=O)(=O)C(F)(F)F)n1 ; O=C(Cc1ccc(OCCN2CCCC2)cc1)Nc1cc(CS(=O)(=O)n2nnc3ccccc32)[nH]n1]

In certain embodiments the heteroaryl sulfonyl compound of the present invention is selected from

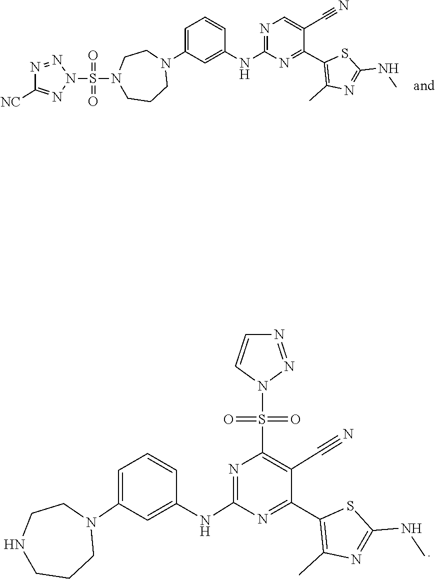 [SMILES: CNc1nc(C)c(-c2nc(Nc3cccc(N4CCCN(S(=O)(=O)n5nnc(C#N)n5)CC4)c3)ncc2C#N)s1 ; CNc1nc(C)c(-c2nc(Nc3cccc(N4CCCNCC4)c3)nc(S(=O)(=O)n3ccnn3)c2C#N)s1]

In certain embodiments the heteroaryl sulfonyl compound of the present invention is selected from

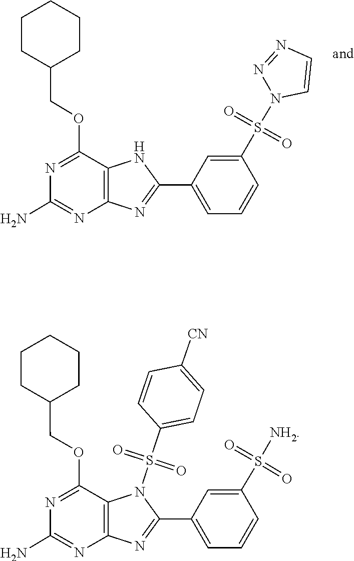 [SMILES: Nc1nc(OCC2CCCCC2)c2[nH]c(-c3cccc(S(=O)(=O)n4ccnn4)c3)nc2n1 ; N#Cc1ccc(S(=O)(=O)n2c(-c3cccc(S(N)(=O)=O)c3)nc3nc(N)nc(OCC4CCCCC4)c32)cc1]

In certain embodiments the heteroaryl sulfonyl compound of the present invention is selected from

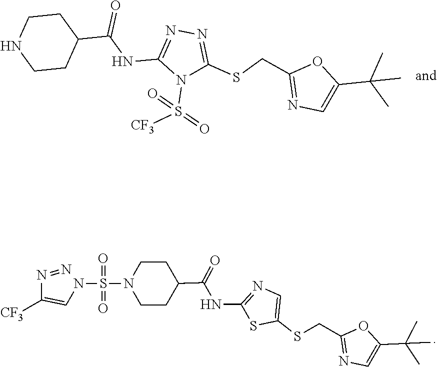

In certain embodiments the heteroaryl sulfonyl compound of the present invention is selected from

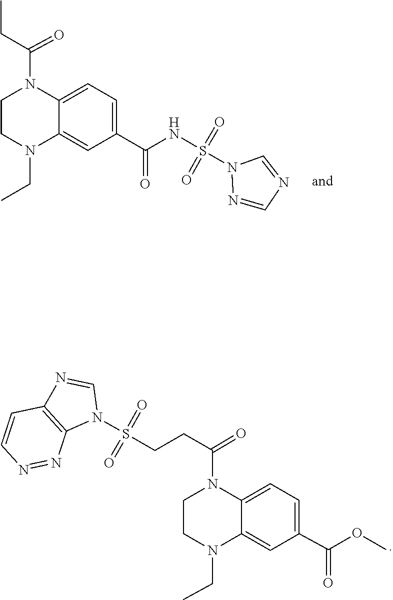 [SMILES: CCC(=O)N1CCN(CC)c2cc(C(=O)NS(=O)(=O)n3cncn3)ccc21 ; CCN1CCN(C(=O)CCS(=O)(=O)n2cnc3ccnnc32)c2ccc(C(=O)OC)cc21]

In certain embodiments the heteroaryl sulfonyl compound of the present invention is selected from

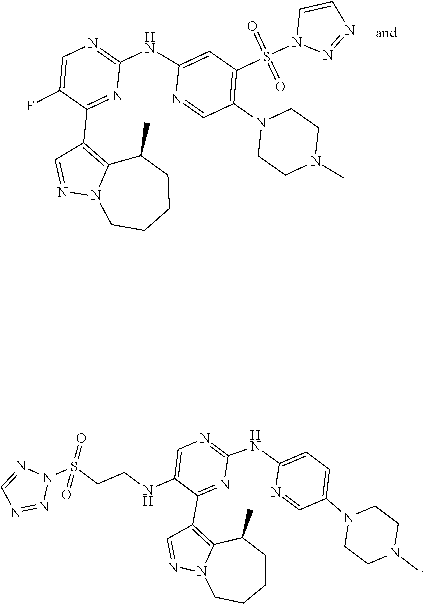 [SMILES: C[C@H]1CCCCn2ncc(-c3nc(Nc4cc(S(=O)(=O)n5ccnn5)c(N5CCN(C)CC5)cn4)ncc3F)c21 ; C[C@H]1CCCCn2ncc(-c3nc(Nc4ccc(N5CCN(C)CC5)cn4)ncc3NCCS(=O)(=O)n3ncnn3)c21]

In certain embodiments the heteroaryl sulfonyl compound of the present invention is selected from

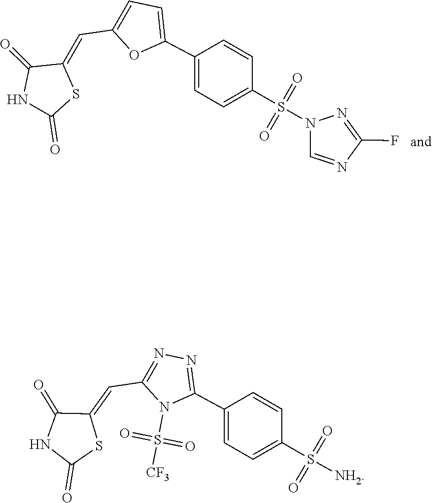 [SMILES: O=C1NC(=O)/C(=C/c2ccc(-c3ccc(S(=O)(=O)n4cnc(F)n4)cc3)o2)S1 ; NS(=O)(=O)c1ccc(-c2nnc(/C=C3\SC(=O)NC3=O)n2S(=O)(=O)C(F)(F)F)cc1]

In certain embodiments the heteroaryl sulfonyl compound of the present invention is selected from

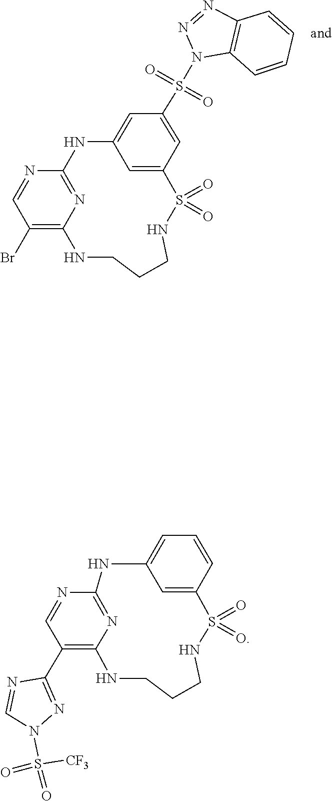 [SMILES: O=S1(=O)NCCCNc2nc(ncc2Br)Nc2cc1cc(S(=O)(=O)n1nnc3ccccc31)c2 ; O=S1(=O)NCCCNc2nc(ncc2-c2ncn(S(=O)(=O)C(F)(F)F)n2)Nc2cccc1c2]

In certain embodiments the heteroaryl sulfonyl compound of the present invention is selected from

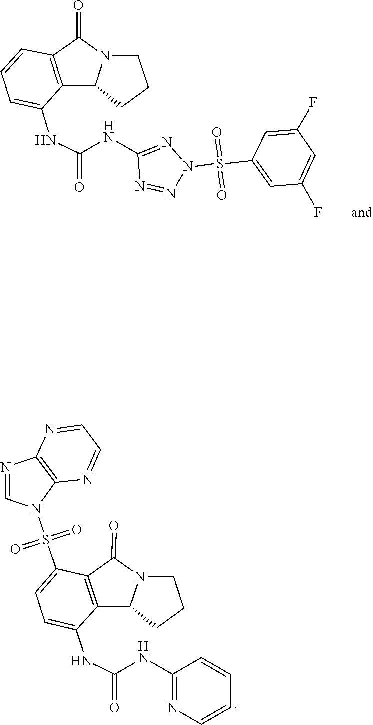 [SMILES: O=C(Nc1nnn(S(=O)(=O)c2cc(F)cc(F)c2)n1)Nc1cccc2c1[C@H]1CCCN1C2=O ; O=C(Nc1ccccn1)Nc1ccc(S(=O)(=O)n2cnc3nccnc32)c2c1[C@H]1CCCN1C2=O]

In certain embodiments the heteroaryl sulfonyl compound of the present invention is selected from

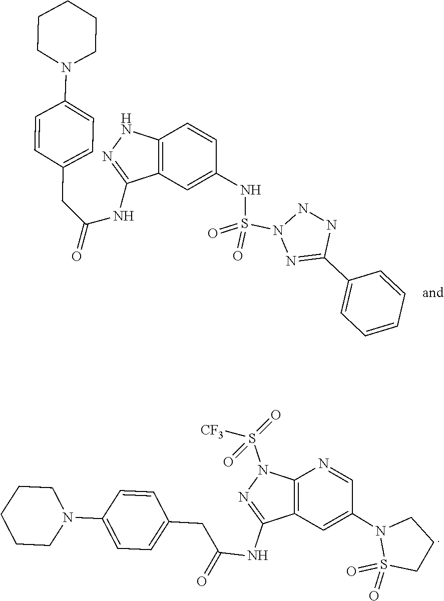 [SMILES: O=C(Cc1ccc(N2CCCCC2)cc1)Nc1n[nH]c2ccc(NS(=O)(=O)N3N=C(c4ccccc4)NN3)cc12 ; O=C(Cc1ccc(N2CCCCC2)cc1)Nc1nn(S(=O)(=O)C(F)(F)F)c2ncc(N3CCCS3(=O)=O)cc12]

In certain embodiments the heteroaryl sulfonyl compound of the present invention is selected from

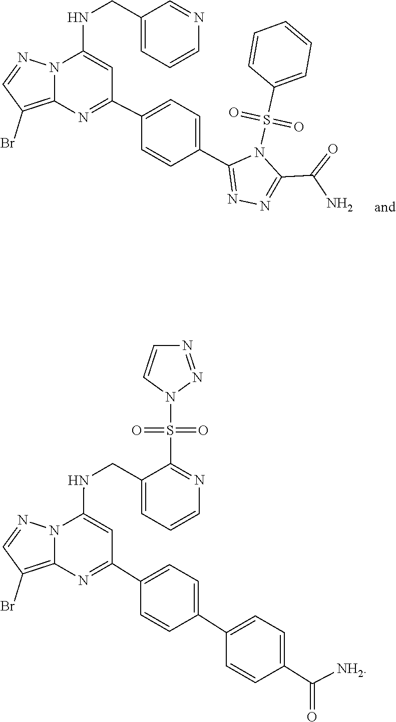 [SMILES: NC(=O)c1nnc(-c2ccc(-c3cc(NCc4cccnc4)n4ncc(Br)c4n3)cc2)n1S(=O)(=O)c1ccccc1 ; NC(=O)c1ccc(-c2ccc(-c3cc(NCc4cccnc4S(=O)(=O)n4ccnn4)n4ncc(Br)c4n3)cc2)cc1]

### Terminology

As used herein, Anchor Bond is defined as the chemical bond between the CDK2 Recognition Moiety and the rest of the molecule for example a bond to R³, R⁹, or R¹⁶, as appropriate. Non-limiting examples of Anchor Bonds are shown in bold in the following structures:

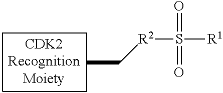

where R³ is methylene,

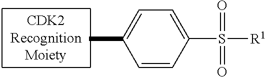

where R³ is bond and R² is phenyl,

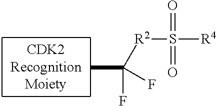

where R⁹ is difluoromethylene, and

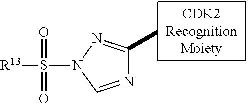

where R¹⁶ is 1,2,4-triazole.

Compounds are described using standard nomenclature. Unless defined otherwise, all technical and scientific terms used herein have the same meaning as is commonly understood by one of skill in the art to which this invention belongs.

The heteroaryl sulfonyl compounds in any of the Formulas described herein include enantiomers, mixtures of enantiomers, diastereomers, tautomers, racemates and other isomers, such as rotamers, as if each is specifically described, unless otherwise indicated or otherwise excluded by context.

The terms “a” and “an” do not denote a limitation of quantity, but rather denote the presence of at least one of the referenced item. The term “or” means “and/or”. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of examples, or exemplary language (e.g., “such as”), is intended merely to better illustrate the invention and does not pose a limitation on the scope of the invention unless otherwise claimed. Unless defined otherwise, technical and scientific terms used herein have the same meaning as is commonly understood by one of skill in the art to which this invention belongs.

In certain embodiments the present invention includes heteroaryl sulfonyl compounds with at least one desired isotopic substitution of an atom, at an amount above the natural abundance of the isotope, i.e., enriched. In certain embodiments the present invention includes heteroaryl sulfonyl compounds that are not isotopically labeled.

Examples of isotopes that can be incorporated into heteroaryl sulfonyl compounds of the invention include isotopes of hydrogen, carbon, nitrogen, oxygen, phosphorous, fluorine, and chlorine, such as ²H, ³H, ¹¹C, ¹³C, ¹⁴C, ¹⁵N, ¹⁷O, ¹⁸O, ¹⁸F ³¹P, ³²P, ³⁵S, ³⁶Cl, and ¹²⁵I respectively. In one embodiment, isotopically labelled heteroaryl sulfonyl compounds can be used in metabolic studies (with, for example ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. For example, a ¹⁸F labeled heteroaryl sulfonyl compound may be desirable for PET or SPECT studies. Isotopically labeled heteroaryl sulfonyl compounds of this invention and prodrugs thereof can generally be prepared by carrying out the procedures disclosed in the schemes or in the examples and preparations described below by substituting a readily available isotopically labeled reagent for a non-isotopically labeled reagent.

By way of general example and without limitation, isotopes of hydrogen, for example, deuterium (²H) and tritium (³H) may optionally be used anywhere in described structures that achieves the desired result. Alternatively, or in addition, isotopes of carbon, e.g., ¹³C and ¹⁴C, may be used. In one embodiment, the isotopic substitution is replacing hydrogen with a deuterium at one or more locations on the molecule to improve the performance of the drug, for example, the pharmacodynamics, pharmacokinetics, biodistribution, half-life, stability, AUC, Tmax, Cmax, etc. For example, the deuterium can be bound to carbon in a location of bond breakage during metabolism (an α-deuterium kinetic isotope effect) or next to or near the site of bond breakage (a β-deuterium kinetic isotope effect).

Isotopic substitutions, for example deuterium substitutions, can be partial or complete. Partial deuterium substitution means that at least one hydrogen is substituted with deuterium. In certain embodiments, the isotope is 80, 85, 90, 95 or 99% or more enriched in an isotope at any location of interest. In certain embodiments deuterium is 80, 85, 90, 95 or 99% enriched at a desired location. Unless otherwise stated, the enrichment at any point is above natural abundance, and in an embodiment is enough to alter a detectable property of the drug in a human.

In one embodiment, the substitution of a hydrogen atom for a deuterium atom occurs within any variable group. For example, when any variable group is, or contain for example through substitution, methyl, ethyl, or methoxy, the alkyl residue may be deuterated (in nonlimiting embodiments, CDH₂, CD₂H, CD₃, CD₂CD₃, CHDCH₂D, CH₂CD₃, CHDCHD₂, OCDH₂, OCD₂H, or OCD₃ etc.).

The heteroaryl sulfonyl compound of the present invention may form a solvate with solvents (including water). Therefore, in one embodiment, the invention includes a solvated form of the active heteroaryl sulfonyl compound. The term “solvate” refers to a molecular complex of a heteroaryl sulfonyl compound of the present invention (including a salt thereof) with one or more solvent molecules. Nonlimiting examples of solvents are water, ethanol, dimethyl sulfoxide, acetone and other common organic solvents. The term “hydrate” refers to a molecular complex comprising a heteroaryl sulfonyl compound of the invention and water. Pharmaceutically acceptable solvates in accordance with the invention include those wherein the solvent of crystallization may be isotopically substituted, e.g. D₂O, d₆-acetone, d₆-DMSO. A solvate can be in a liquid or solid form.

A dash (“-”) that is not between two letters or symbols is used to indicate a point of attachment for a substituent. For example, —(C═O)NH₂ is attached through carbon of the keto (C═O) group.

The term “substituted”, as used herein, means that any one or more hydrogens on the designated atom or group is replaced with a moiety selected from the indicated group, provided that the designated atom's normal valence is not exceeded and the resulting heteroaryl sulfonyl compound is stable. For example, when the substituent is oxo (i.e., ═O) then two hydrogens on the atom are replaced. For example a pyridyl group substituted by oxo is a pyridone. Combinations of substituents and/or variables are permissible only if such combinations result in stable heteroaryl sulfonyl compounds or useful synthetic intermediates.

“Alkyl” is a branched, straight chain, or cyclic saturated aliphatic hydrocarbon group. In one embodiment, the alkyl contains from 1 to about 12 carbon atoms, more generally from 1 to about 6 carbon atoms, from 1 to about 4 carbon atoms, or from 1 to 3 carbon atoms. In one embodiment, the alkyl contains from 1 to about 8 carbon atoms. In certain embodiments, the alkyl is C₁-C₂, C₁-C₃, C₁-C₄, C₁-C₅ or C₁-C₆. The specified ranges as used herein indicate an alkyl group which is considered to explicitly disclose as individual species each member of the range described as a unique species. For example, the term C₁-C₆ alkyl as used herein indicates a straight or branched alkyl group having from 1, 2, 3, 4, 5, or 6 carbon atoms and also a carbocyclic alkyl group of 3, 4, 5, or 6 carbon atoms and is intended to mean that each of these is described as an independent species. For example, the term C₁-C₄alkyl as used herein indicates a straight or branched alkyl group having from 1, 2, 3, or 4 carbon atoms and is intended to mean that each of these is described as an independent species. When C₀-Cₙ alkyl is used herein in conjunction with another group, for example, (C₃₋C₇cycloalkyl)C₀-C₄ alkyl, or —C₀-C₄alkyl(C₃-C₇cycloalkyl), the indicated group, in this case cycloalkyl, is either directly bound by a single covalent bond (C₀alkyl), or attached by an alkyl chain in this case 1, 2, 3, or 4 carbon atoms. Alkyls can also be attached via other groups such as heteroatoms as in —O—C₀-C₄alkyl(C₃-C₇cycloalkyl). Examples of alkyl include, but are not limited to, methyl, ethyl, n-propyl, isopropyl, n-butyl, isobutyl, sec-butyl, t-butyl, n-pentyl, isopentyl, tert-pentyl, neopentyl, n-hexyl, 2-methylpentane, 3-methylpentane, 2,2-dimethylbutane, 2,3-dimethylbutane, and hexyl.

When a term is used that includes “alk” it should be understood that “cycloalkyl” or “carbocyclic” can be considered part of the definition, unless unambiguously excluded by the context. For example and without limitation, the terms alkyl, alkenyl, alkynyl, alkoxy, alkanoyl, alkenloxy, haloalkyl, etc. can all be considered to include the cyclic forms of alkyl, unless unambiguously excluded by context.

“Alkenyl” is a branched or straight chain aliphatic hydrocarbon group having one or more carbon-carbon double bonds that may occur at a stable point along the chain. Nonlimiting examples are C₂-C₈alkenyl, C₂-C₇alkenyl, C₂-C₆alkenyl, C₂-C₅alkenyl and C₂-C₄alkenyl. The specified ranges as used herein indicate an alkenyl group having each member of the range described as an independent species, as described above for the alkyl moiety. Examples of alkenyl include, but are not limited to, ethenyl and propenyl.

“Alkynyl” is a branched or straight chain aliphatic hydrocarbon group having one or more carbon-carbon triple bonds that may occur at any stable point along the chain, for example, C₂-C₈alkynyl or C₂-C₆alkynyl. The specified ranges as used herein indicate an alkynyl group having each member of the range described as an independent species, as described above for the alkyl moiety. Examples of alkynyl include, but are not limited to, ethynyl, propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl and 5-hexynyl.

“Alkoxy” is an alkyl group as defined above covalently bound through an oxygen bridge (—O—). Examples of alkoxy include, but are not limited to, methoxy, ethoxy, n-propoxy, i-propoxy, n-butoxy, 2-butoxy, t-butoxy, n-pentoxy, 2-pentoxy, 3-pentoxy, isopentoxy, neopentoxy, n-hexoxy, 2-hexoxy, 3-hexoxy, and 3-methylpentoxy. Similarly an “alkylthio” or a “thioalkyl” group is an alkyl group as defined above with the indicated number of carbon atoms covalently bound through a sulfur bridge (—S—). In one embodiment, the alkoxy group is optionally substituted as described above.

“Haloalkyl” indicates both branched and straight-chain alkyl groups substituted with 1 or more halogen atoms, up to the maximum allowable number of halogen atoms. Examples of haloalkyl include, but are not limited to, trifluoromethyl, monofluoromethyl, difluoromethyl, 2-fluoroethyl, and penta-fluoroethyl.

“Aryl” indicates an aromatic group containing only carbon in the aromatic ring or rings. In one embodiment, the aryl group contains 1 to 3 separate or fused rings and is 6 to 14 or 18 ring atoms, without heteroatoms as ring members. The term “aryl” includes groups where a saturated or partially unsaturated carbocycle group is fused with an aromatic ring. The term “aryl” also includes groups where a saturated or partially unsaturated heterocycle group is fused with an aromatic ring so long as the attachment point is the aromatic ring. Such heteroaryl sulfonyl compounds may include aryl rings fused to a 4 to 7 or a 5 to 7-membered saturated or partially unsaturated cyclic group that optionally contains 1, 2 or 3 heteroatoms independently selected from N, O, B, P, Si and S, to form, for example, a 3,4-methylenedioxyphenyl group. Aryl groups include, for example, phenyl and naphthyl, including 1-naphthyl and 2-naphthyl. In one embodiment, aryl groups are pendant. An example of a pendant ring is a phenyl group substituted with a phenyl group.

The term “heterocycle” refers to saturated and partially saturated heteroatom-containing ring radicals, where the heteroatoms may be selected from N, S, and O. The term “heterocycle” includes monocyclic 3-12 membered rings, as well as bicyclic 5-16 membered ring systems (which can include fused, bridged, or spiro, bicyclic ring systems). It does not include rings containing —O—O— or —S—S— portions. Examples of saturated heterocycle groups include saturated 4- to 7-membered monocyclic groups containing 1 to 4 nitrogen atoms [e.g., pyrrolidinyl, imidazolidinyl, piperidinyl, pyrrolinyl, azetidinyl, piperazinyl, and pyrazolidinyl]; saturated 4 to 6-membered monocyclic groups containing 1 to 2 oxygen atoms and 1 to 3 nitrogen atoms [e.g., morpholinyl]; saturated 3 to 6-membered heteromonocyclic group containing 1 to 2 sulfur atoms and 1 to 3 nitrogen atoms [e.g., thiazolidinyl]. Examples of partially saturated heterocycle radicals include but are not limited to, dihydrothienyl, dihydropyranyl, dihydrofuryl, and dihydrothiazolyl. Examples of partially saturated and saturated heterocycle groups include but are not limited to, pyrrolidinyl, imidazolidinyl, piperidinyl, pyrrolinyl, pyrazolidinyl, piperazinyl, morpholinyl, tetrahydropyranyl, thiazolidinyl, dihydrothienyl, 2,3-dihydro-benzo[1,4]dioxanyl, indolinyl, isoindolinyl, dihydrobenzothienyl, dihydrobenzofuryl, isochromanyl, chromanyl, 1,2-dihydroquinolyl, 1,2,3,4-tetrahydro-isoquinolyl, 1,2,3,4-tetrahydro-quinolyl, 2,3,4,4a,9,9a-hexahydro-1H-3-aza-fluorenyl, 5,6,7-trihydro-1,2,4-triazolo[3,4-a]isoquinolyl, 3,4-dihydro-2H-benzo[1,4]oxazinyl, benzo[1,4]dioxanyl, 2,3-dihydro-1H-1λ′-benzo[d]isothiazol-6-yl, dihydropyranyl, dihydrofuryl and dihydrothiazolyl. “Bicyclic heterocycle” includes groups wherein the heterocyclic radical is fused with an aryl radical wherein the point of attachment is the heterocycle ring. “Bicyclic heterocycle” also includes heterocyclic radicals that are fused or bridged with a carbocycle radical. For example partially unsaturated condensed heterocyclic group containing 1 to 5 nitrogen atoms, for example, indoline, isoindoline, partially unsaturated condensed heterocyclic group containing 1 to 2 oxygen atoms and 1 to 3 nitrogen atoms, partially unsaturated condensed heterocyclic group containing 1 to 2 sulfur atoms and 1 to 3 nitrogen atoms, and saturated condensed heterocyclic group containing 1 to 2 oxygen or sulfur atoms.

Non-limiting examples of bicyclic heterocycles include:

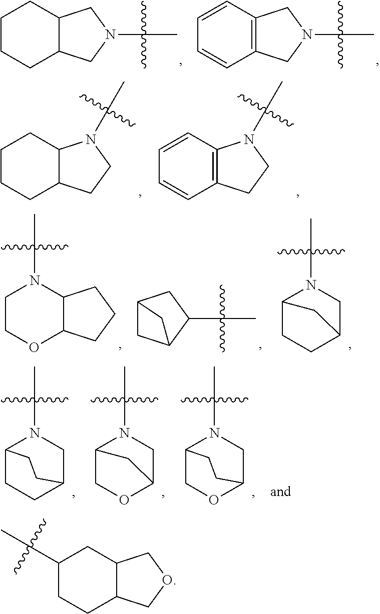

Unless otherwise drawn or clear from the context, the term “bicyclic heterocycle” includes cis and trans diastereomers. Non-limiting examples of chiral bicyclic heterocycles include:

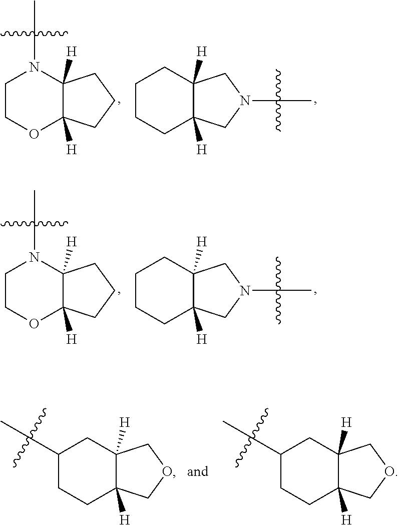

In certain alternative embodiments the term “heterocycle” refers to saturated and partially saturated heteroatom-containing ring radicals, where the heteroatoms may be selected from N, S, O, B, Si, and P.

The term “bicycle” refers to a ring system wherein two rings are fused together and each ring is independently selected from carbocycle, heterocycle, aryl, and heteroaryl. Non-limiting examples of bicycle groups include:

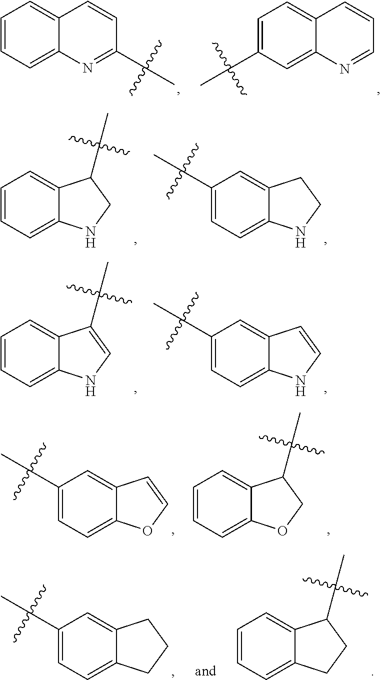

When the term “bicycle” is used in the context of a bivalent residue such as R², R³, or R⁵, the attachment points can be on separate rings or on the same ring. In certain embodiments both attachment points are on the same ring. In certain embodiments both attachment points are on different rings. Non-limiting examples of bivalent bicycle groups include:

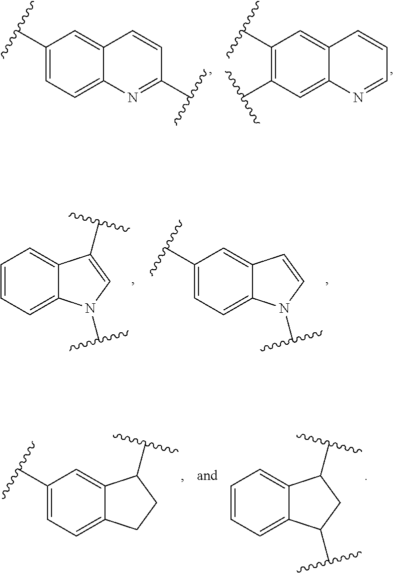 [SMILES: *c1ccc2nc(*)ccc2c1 ; *c1cc2cccnc2cc1* ; *c1cn(*)c2ccccc12 ; *c1ccc2c(ccn2*)c1 ; *c1ccc2c(c1)C(*)CC2 ; *C1CC(*)c2ccccc21]

The term “tricycle” refers to a ring system wherein three rings are fused together and each ring is independently selected from carbocycle, heterocycle, aryl, and heteroaryl. Non-limiting examples of bicycle groups include:

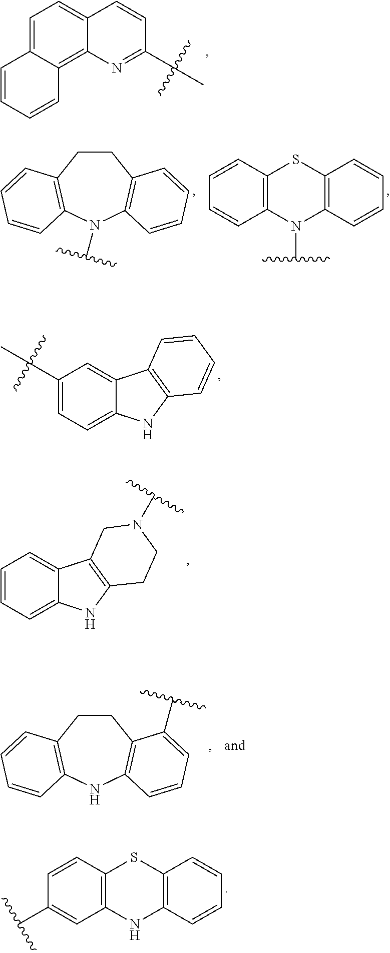

When the term “tricycle” is used in the context of a bivalent residue such as R², R³, or R⁵, the attachment points can be on separate rings or on the same ring. In certain embodiments both attachment points are on the same ring. In certain embodiments both attachment points are on different rings. Non-limiting examples of bivalent tricycle groups include:

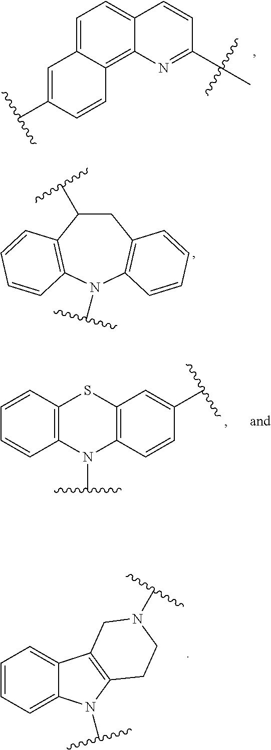 [SMILES: *c1ccc2c(ccc3ccc(*)nc32)c1 ; *C1Cc2ccccc2N(*)c2ccccc21 ; *c1ccc2c(c1)Sc1ccccc1N2* ; *N1CCc2c(c3ccccc3n2*)C1]

“Heteroaryl” refers to a stable monocyclic, bicyclic, or multicyclic aromatic ring which contains from 1 to 5, or in some embodiments from 1, 2, or 3 heteroatoms selected from N, O, S, B, and P (and typically selected from N, O, and S) with remaining ring atoms being carbon, or a stable bicyclic or tricyclic system containing at least one 5, 6, or 7 membered aromatic ring which contains from 1 to 3, or in some embodiments from 1 to 2, heteroatoms selected from N, O, S, B or P with remaining ring atoms being carbon. In one embodiment, the only heteroatom is nitrogen.

In one embodiment, the only heteroatom is oxygen. In one embodiment, the only heteroatom is sulfur. Monocyclic heteroaryl groups typically have from 5 or 6 ring atoms. In some embodiments bicyclic heteroaryl groups are 8- to 10-membered heteroaryl groups, that is, groups containing 8 or 10 ring atoms in which one 5, 6, or 7-member aromatic ring is fused to a second aromatic or non-aromatic ring wherein the point of attachment is the aromatic ring. When the total number of S and O atoms in the heteroaryl group exceeds 1, these heteroatoms are not adjacent to one another. In one embodiment, the total number of S and O atoms in the heteroaryl group is not more than 2. In another embodiment, the total number of S and O atoms in the aromatic heterocycle is not more than 1. Examples of heteroaryl groups include, but are not limited to, pyridinyl (including, for example, 2-hydroxypyridinyl), imidazolyl, imidazopyridinyl, pyrimidinyl (including, for example, 4-hydroxypyrimidinyl), pyrazolyl, triazolyl, pyrazinyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, triazolyl, thiadiazolyl, thiadiazolyl, furazanyl, benzofurazanyl, benzothiophenyl, benzothiazolyl, benzoxazolyl, quinazolinyl, quinoxalinyl, naphthyridinyl, tetrahydrofuranyl, and furopyridinyl. Heteroaryl groups are optionally substituted independently with one or more substituents described herein.

A “dosage form” means a unit of administration of an active agent. Examples of dosage forms include tablets, capsules, injections, suspensions, liquids, emulsions, implants, particles, spheres, creams, ointments, suppositories, inhalable forms, transdermal forms, buccal, sublingual, topical, gel, mucosal, and the like. A “dosage form” can also include an implant, for example an optical implant.

“Pharmaceutical compositions” are compositions comprising at least one active agent, and at least one other substance, such as a carrier. The present invention includes pharmaceutical compositions of the described compounds.

“Pharmaceutical combinations” are combinations of at least two active agents which may be combined in a single dosage form or provided together in separate dosage forms with instructions that the active agents are to be used together to treat any disorder described herein.

A “pharmaceutically acceptable salt” is a derivative of the disclosed heteroaryl sulfonyl compound in which the parent heteroaryl sulfonyl compound is modified by making inorganic and organic, pharmaceutically acceptable, acid or base addition salts thereof. The salts of the present heteroaryl sulfonyl compounds can be synthesized from a parent heteroaryl sulfonyl compound that contains a basic or acidic moiety by conventional chemical methods. Generally, such salts can be prepared by reacting free acid forms of these heteroaryl sulfonyl compounds with a stoichiometric amount of the appropriate base (such as Na, Ca, Mg, or K hydroxide, carbonate, bicarbonate, or the like), or by reacting free base forms of these heteroaryl sulfonyl compounds with a stoichiometric amount of the appropriate acid. Such reactions are typically carried out in water or in an organic solvent, or in a mixture of the two. Salts of the present heteroaryl sulfonyl compounds further include solvates of the heteroaryl sulfonyl compounds and of the heteroaryl sulfonyl compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include salts which are acceptable for human consumption and the quaternary ammonium salts of the parent heteroaryl sulfonyl compound formed, for example, from inorganic or organic acids. Examples, of such salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)₁₋₄—COOH, and the like, or using a different acid that produces the same counterion. Lists of additional suitable salts may be found, e.g., in Remington's Pharmaceutical Sciences, 17th ed., Mack Publishing Company, Easton, Pa., p. 1418 (1985).

The term “carrier” applied to pharmaceutical compositions/combinations of the invention refers to a diluent, excipient, or vehicle with which an active heteroaryl sulfonyl compound is provided.

A “pharmaceutically acceptable excipient” means an excipient that is useful in preparing a pharmaceutical composition/combination that is generally safe, acceptable for human consumption, and neither biologically nor otherwise inappropriate for administration to a host, typically a human. In one embodiment, an excipient is used that is acceptable for veterinary use.

A “patient” or “host” or “subject” is a human or non-human animal in need of treatment or prevention of any of the disorders as specifically described herein. Typically, the host is a human.

A “patient” or “host” or “subject” also refers to for example, a mammal, primate (e.g., human), cow, sheep, goat, horse, dog, cat, rabbit, rat, mice, bird and the like.

A “therapeutically effective amount” of a compound, pharmaceutical composition, or combination of this invention means an amount effective, when administered to a host, provides a therapeutic benefit such as an amelioration of symptoms or reduction or diminution of the disease itself.

### CDK2 Recognition Moiety

CDK2 recognition moiety is a molecule, for example a small molecule, peptide, protein, oligonucleotide, nucleotide, RNA, DNA, SiRNA, a biologic, an antibody, or a fragment thereof, which can bind to or otherwise interact with CDK2.

In non-limiting embodiments, the CDK2 Recognition Moiety is a protein binding domain of a drug or pharmaceutically active compound which modulates CDK2 (or the full drug or pharmaceutically active compound bound as part of the compound of the present invention).

Alternatively, the CDK2 Recognition Moiety is a peptide, protein, oligonucleotide, nucleotide, RNA, DNA, SiRNA, miRNA, a biologic, receptor binding domain, an antibody or a fragment thereof, which can bind to or otherwise interact with CDK2 to anchor the compound of the present invention to CDK2.

The CDK2 Recognition Moiety is typically a ligand or a portion of a ligand that binds to the CDK2. Non-limiting examples of CDK2 Recognition Moieties are provided below.

Additionally CDK2 Recognition Moieties are known in the art and where to link the moiety to provide the desired effect can readily be determined. For example, the crystal structure for CDK2 can be accessed on https://www.rcsb.org/ and then a list of ligands that bind that crystal structure can be generated. Where to attach the sulfur-heteroaryl group of the present invention can be determined based on the crystal structure provided which will allow identification of where in the binding pocket the sulfur-heteroaryl group can fit and which functional groups on the ligand are essential for activity.

As used herein codes referring to crystal structures correspond to the crystal structure available on the Protein Data Bank (PDB, https://www.rcsb.org). For example, non-limiting examples of crystal structures of CDK2 include 1B39; 1GU; 1GII; 1GIH 2C5Y; 1BUH; 1B38; 3PXY; 3PXR; 6INL; 2C50; 4EON; and 4E00 where these codes correspond to crystal structures in the PDB database and available online at https://www.rcsb.org.

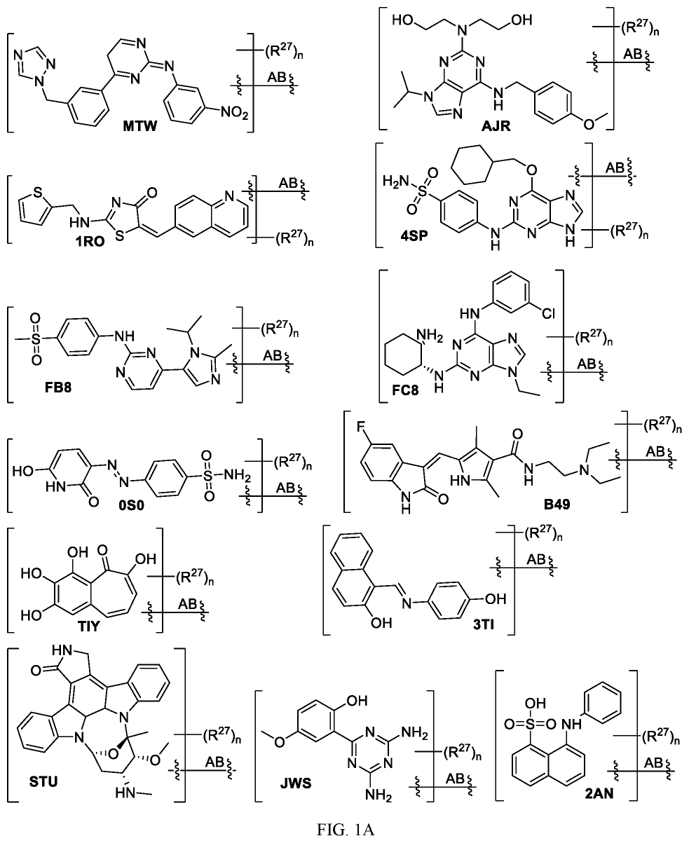

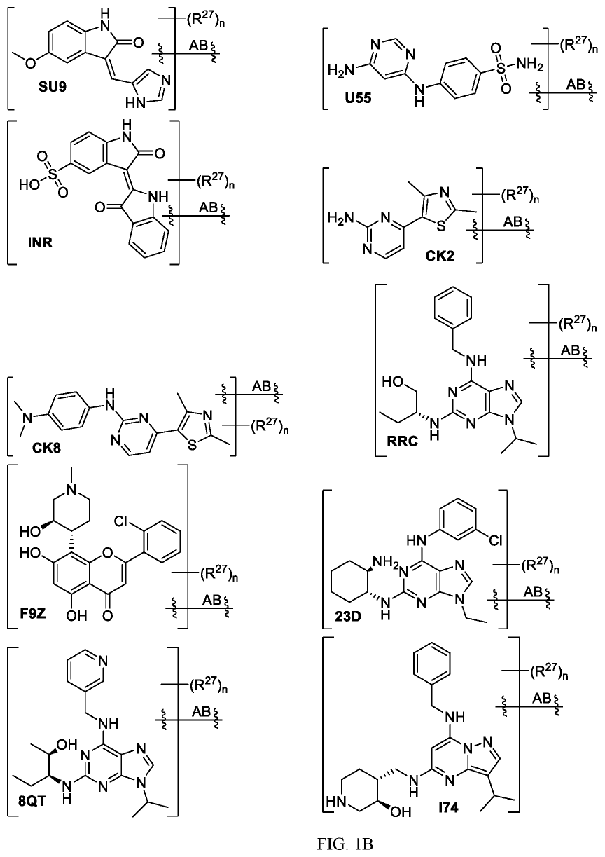

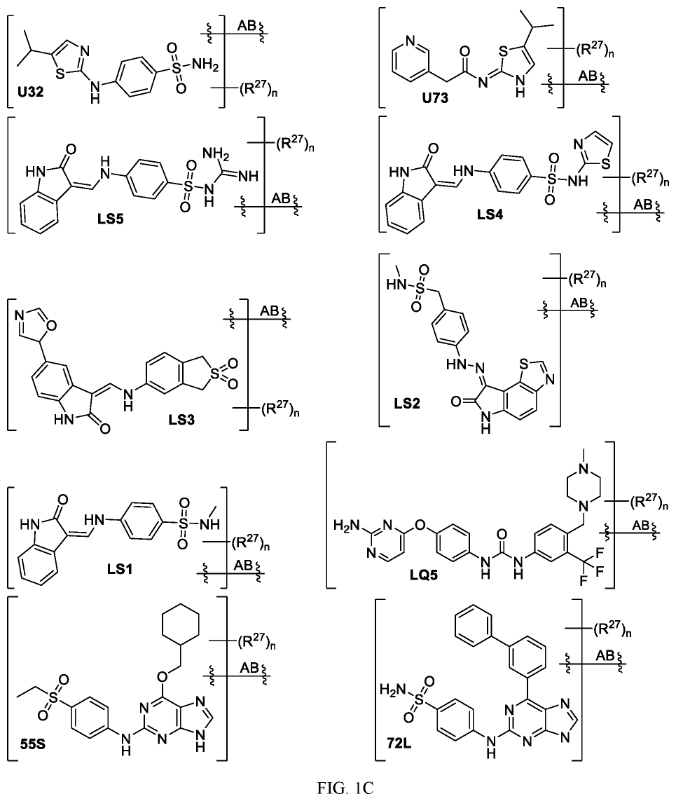

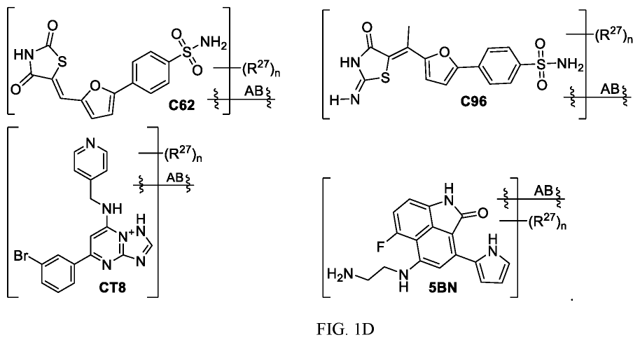

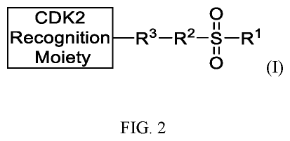

In certain embodiments, the CDK2 Recognition Moiety is a ligand that binds cyclin dependent kinase 2 (CDK2). Non-limiting examples of crystal structures of CDK2 with CDK2 Recognition Moieties include 2C5Y, 6INL, 4EON, 4EOR, 6GUH, 6GUK, 4EZ3, 3TI1, 3TIY, 3TIZ, 4ERW, 3PXY, 3PXZ, 3PY0, 1JSV, 1E9H, 2C5O, 2C5N, 2A4L, 6GUB, 6GUF, 5MHQ, 5JQ5, 2BTS, 2BTR, 1KE9, 1KE8, 1KE7, 1KE6, 1KE5, 5A14, 5CYI, 5NEV, 5LQF, 2UZO, 2UZN, 2C69 and 1P2A. Representative CDK2 Targeting Ligands are provided in FIGS. 1A, 1B, 1C, and ID.

As used herein R²⁷ is independently selected at each instance from the group consisting of hydrogen, halogen, alkyl, haloalkyl, alkenyl, cycloalkyl, heterocycle, aryl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, —S(O)R⁶, —S(O)₂R⁶, —S(O)OR⁶, —S(O)₂OR⁶, —S(O)N(R⁶)₂, S(O)₂N(R⁶)₂, ═O, and —SR⁶, wherein each alkyl, haloalkyl, alkenyl, cycloalkyl, heterocycle, aryl, and heteroaryl optionally substituted as allowed by valence with 1, 2, 3, or 4 substituents selected from R¹⁷;

As used herein n is 0, 1, 2, 3, or 4.

As used herein

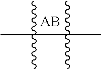 [SMILES: **]

is an Anchor Bond. Anchor Bond is the chemical bond between the CDK2 Recognition Moiety and the rest of the molecule for example a bond to R³, R⁹, or R¹⁶, as appropriate.

In certain embodiments the CDK2 Recognition Moiety is dinaciclib or a fragment or derivative thereof. For example, in certain embodiments, the CDK2 Recognition Moiety is selected from:

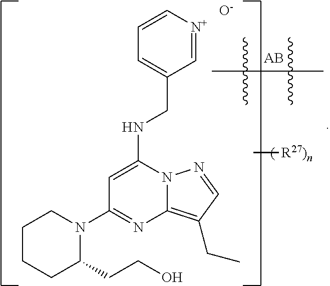

In certain embodiments the CDK2 Recognition Moiety is flavopiridol or a fragment or derivative thereof. For example, in certain embodiments, the CDK2 Recognition Moiety is selected from:

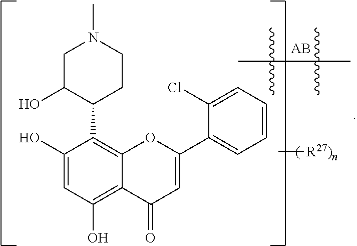

In certain embodiments the CDK2 Recognition Moiety is voruciclib or a fragment or derivative thereof. For example, in certain embodiments, the CDK2 Recognition Moiety is selected from:

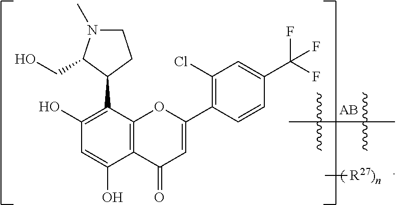

In certain embodiments the CDK2 Recognition Moiety is AZD5438 or a fragment or derivative thereof. For example, in certain embodiments, the CDK2 Recognition Moiety is selected from:

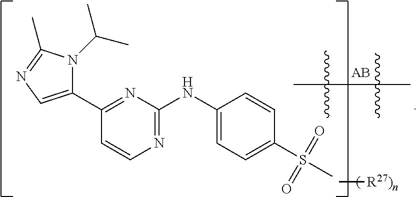

In certain embodiments the CDK2 Recognition Moiety is SNS-032 or a fragment or derivative thereof. For example, in certain embodiments, the CDK2 Recognition Moiety is selected from:

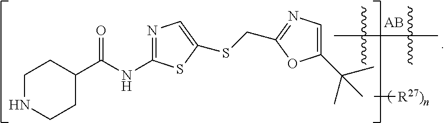

In certain embodiments the CDK2 Recognition Moiety is PHA-690509 or a fragment or derivative thereof. For example, in certain embodiments, the CDK2 Recognition Moiety is selected from:

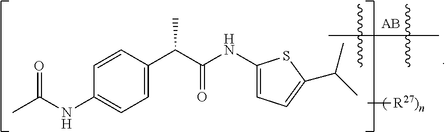

In certain embodiments the CDK2 Recognition Moiety is AT7519 or a fragment or derivative thereof. For example, in certain embodiments, the CDK2 Recognition Moiety is selected

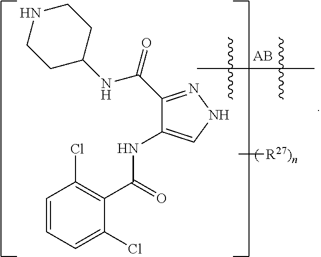

In certain embodiments the CDK2 Recognition Moiety is FN-1501 or a fragment or derivative thereof. For example, in certain embodiments, the CDK2 Recognition Moiety is selected

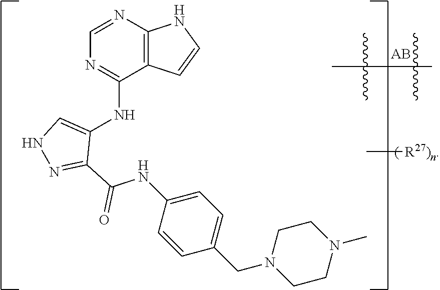

In certain embodiments the CDK2 Recognition Moiety is JNJ-7706621 or a fragment or derivative thereof. For example, in certain embodiments, the CDK2 Recognition Moiety is selected from:

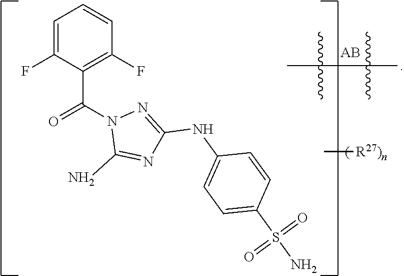

In certain embodiments the CDK2 Recognition Moiety is PF-00562271 or a fragment or derivative thereof. For example, in certain embodiments, the CDK2 Recognition Moiety is selected

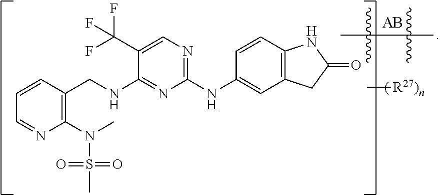

In certain embodiments the CDK2 Recognition Moiety is MK-8776 or a fragment or derivative thereof. For example, in certain embodiments, the CDK2 Recognition Moiety is

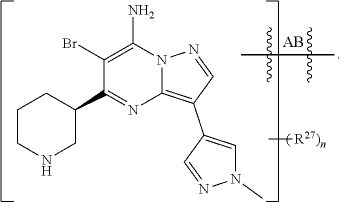

In certain embodiments the CDK2 Recognition Moiety is PHA-793887 or a fragment or derivative thereof. For example, in certain embodiments, the CDK2 Recognition Moiety is selected from:

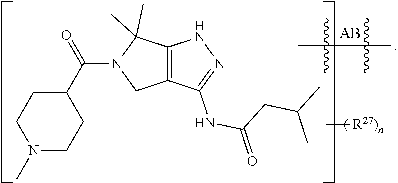

In certain embodiments the CDK2 Recognition Moiety is BMS-265246 or a fragment or derivative thereof. For example, in certain embodiments, the CDK2 Recognition Moiety is selected from:

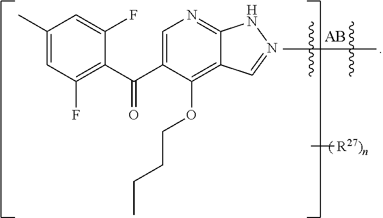

In certain embodiments the CDK2 Recognition Moiety is Milciclib or a fragment or derivative thereof. For example, in certain embodiments, the CDK2 Recognition Moiety is selected from:

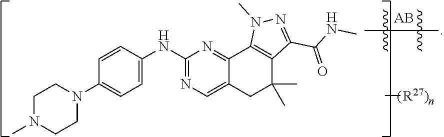

In certain embodiments the CDK2 Recognition Moiety is R547 or a fragment or derivative thereof. For example, in certain embodiments, the CDK2 Recognition Moiety is selected from:

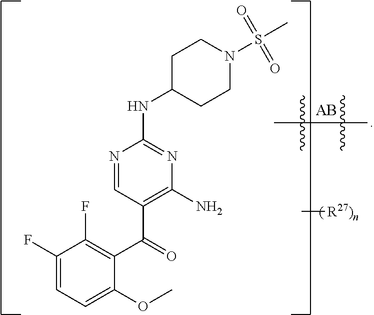

In certain embodiments the CDK2 Recognition Moiety is CDKI-73 or a fragment or derivative thereof. For example, in certain embodiments, the CDK2 Recognition Moiety is selected from:

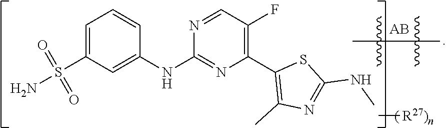

In certain embodiments the CDK2 Recognition Moiety is Purvalanol A or a fragment or derivative thereof. For example, in certain embodiments, the CDK2 Recognition Moiety is selected from:

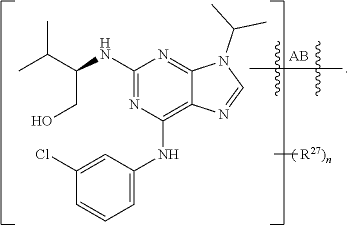

In certain embodiments the CDK2 Recognition Moiety is Purvalanol B or a fragment or derivative thereof. For example, in certain embodiments, the CDK2 Recognition Moiety is selected from:

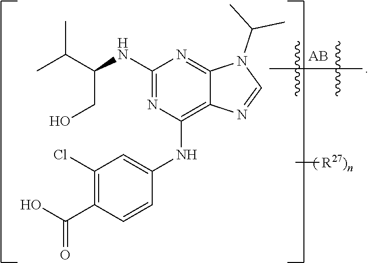

In certain embodiments the CDK2 Recognition Moiety is ZK-304709 or a fragment or derivative thereof. For example, in certain embodiments, the CDK2 Recognition Moiety is selected from:

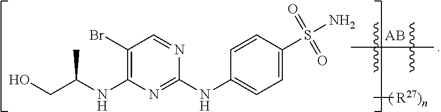

In certain embodiments the CDK2 Recognition Moiety is Roniciclib or a fragment or derivative thereof. For example, in certain embodiments, the CDK2 Recognition Moiety is selected from:

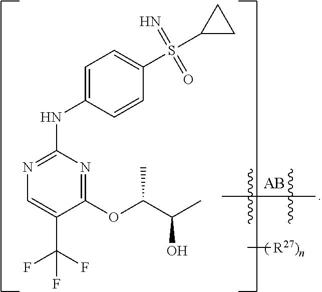

In certain embodiments the CDK2 Recognition Moiety is K03861 or a fragment or derivative thereof. For example, in certain embodiments, the CDK2 Recognition Moiety is selected from:

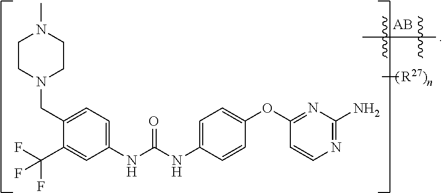

In certain embodiments the CDK2 Recognition Moiety is SU9516 or a fragment or derivative thereof. For example, in certain embodiments, the CDK2 Recognition Moiety is selected from:

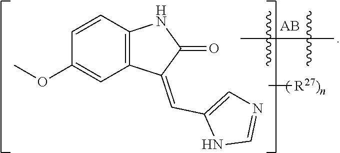

In certain embodiments the CDK2 Recognition Moiety is ANS or a fragment or derivative thereof. For example, in certain embodiments, the CDK2 Recognition Moiety is selected from:

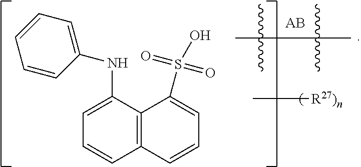

In certain embodiments the CDK2 Recognition Moiety is Meriolin 3 or a fragment or derivative thereof. For example, in certain embodiments, the CDK2 Recognition Moiety is selected

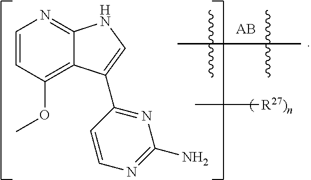

In certain embodiments the CDK2 Recognition Moiety is Fadraciclib or a fragment or derivative thereof. For example, in certain embodiments, the CDK2 Recognition Moiety is selected from:

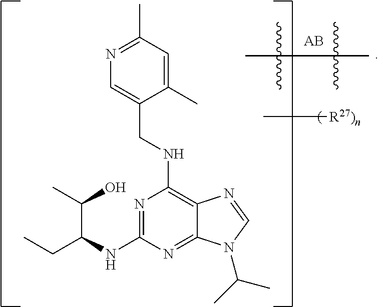

In certain embodiments the CDK2 Recognition Moiety is Seliciclib or a fragment or derivative thereof. For example, in certain embodiments, the CDK2 Recognition Moiety is selected from:

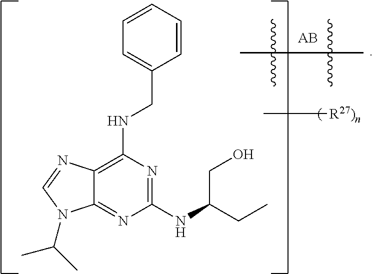

In certain embodiments the CDK2 Recognition Moiety is PF-06873600 or a fragment or derivative thereof. For example, in certain embodiments, the CDK2 Recognition Moiety is selected from:

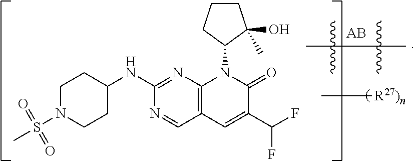

In certain embodiments the CDK2 Recognition Moiety is PF-07104091 or a fragment or derivative thereof.

In certain embodiments the CDK2 Recognition Moiety is 4-methoxy-5-nitro-2-((pyridin-3-ylmethyl)amino)benzamide or a fragment or derivative thereof. For example, in certain embodiments, the CDK2 Recognition Moiety is selected from:

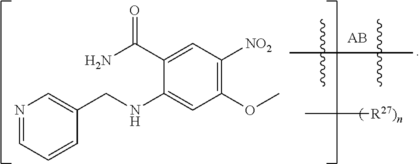

In certain embodiments the CDK2 Recognition Moiety is (Z)—N-((4-(((7-oxo-6,7-dihydro-8H-thiazolo[5,4-e]indol-8-ylidene)methyl)amino)phenyl)sulfonyl)acetamide or a fragment or derivative thereof. For example, in certain embodiments, the CDK2 Recognition Moiety is selected from:

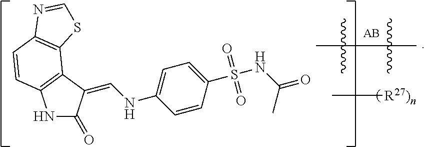

In certain embodiments the CDK2 Recognition Moiety is N-(5-cyclopropyl-1H-pyrazol-3-yl)-2-(4-(2-(pyrrolidin-1-yl)ethoxy)phenyl)acetamide or a fragment or derivative thereof. For example, in certain embodiments, the CDK2 Recognition Moiety is selected from:

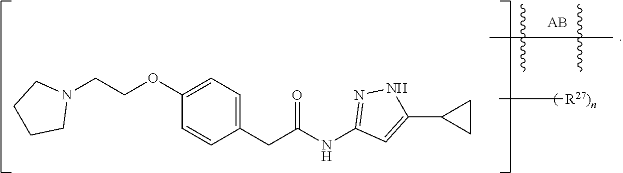

In certain embodiments the CDK2 Recognition Moiety is 2-((3-(1,4-diazepan-1-yl)phenyl)amino)-4-(4-methyl-2-(methylamino)thiazol-5-yl)pyrimidine-5-carbonitrile or a fragment or derivative thereof. For example, in certain embodiments, the CDK2 Recognition Moiety is selected from:

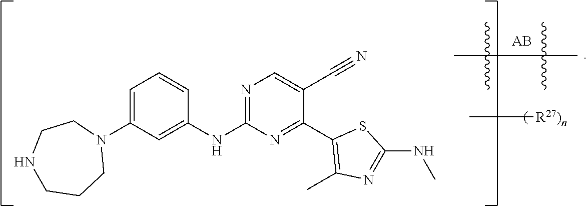

In certain embodiments the CDK2 Recognition Moiety is 3-(2-amino-6-(cyclohexylmethoxy)-7H-purin-8-yl)benzenesulfonamide or a fragment or derivative thereof. For example, in certain embodiments, the CDK2 Recognition Moiety is selected from:

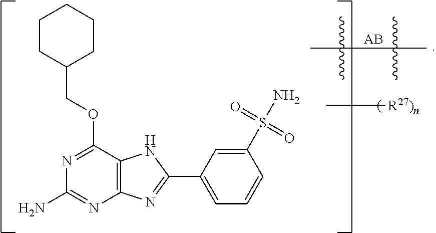

In certain embodiments the CDK2 Recognition Moiety is N-(5-(((5-(tert-butyl)oxazol-2-yl)methyl)thio)thiazol-2-yl)piperidine-4-carboxamide or a fragment or derivative thereof. For example, in certain embodiments, the CDK2 Recognition Moiety is selected from:

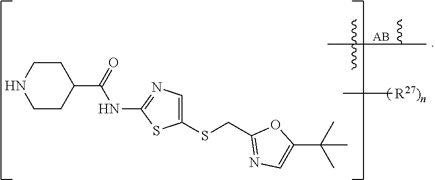

In certain embodiments the CDK2 Recognition Moiety is methyl 4-ethyl-1-propanoyl-2,3-dihydroquinoxaline-6-carboxylate or a fragment or derivative thereof. For example, in certain embodiments, the CDK2 Recognition Moiety is selected from:

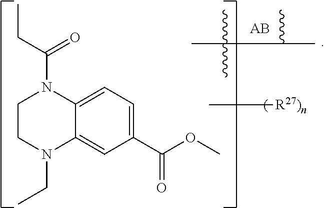

In certain embodiments the CDK2 Recognition Moiety is (S)-5-fluoro-4-(4-methyl-5,6,7,8-tetrahydro-4H-pyrazolo[1,5-a]azepin-3-yl)-N-(5-(4-methylpiperazin-1-yl)pyridin-2-yl)pyrimidin-2-amine or a fragment or derivative thereof. For example, in certain embodiments, the CDK2 Recognition Moiety is selected from:

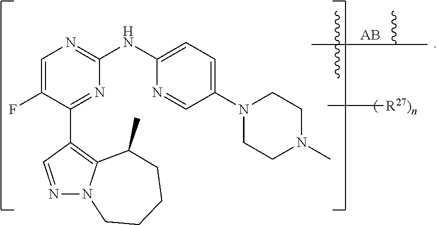

In certain embodiments the CDK2 Recognition Moiety is (Z)-4-(5-((2,4-dioxothiazolidin-5-ylidene)methyl)furan-2-yl)benzenesulfonamide or a fragment or derivative thereof. For example, in certain embodiments, the CDK2 Recognition Moiety is selected from:

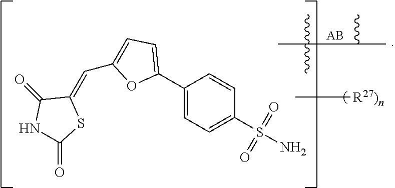

In certain embodiments the CDK2 Recognition Moiety is 15-bromo-4-thia-2,5,9-triaza-1(2,4)-pyrimidina-3(1,3)-benzenacyclononaphane 4,4-dioxide or a fragment or derivative thereof. For example, in certain embodiments, the CDK2 Recognition Moiety is selected from:

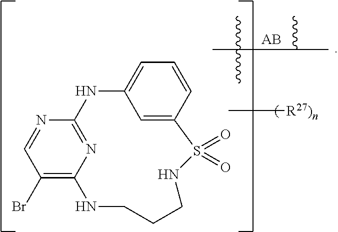

In certain embodiments the CDK2 Recognition Moiety is (R)-1-(5-oxo-2,3,5,9b-tetrahydro-1H-pyrrolo[2,1-a]isoindol-9-yl)-3-(pyridin-2-yl)urea or a fragment or derivative thereof. For example, in certain embodiments, the CDK2 Recognition Moiety is selected from:

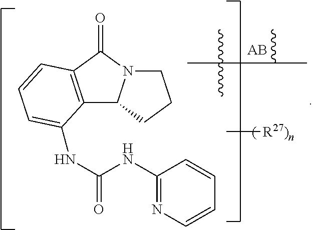

In certain embodiments the CDK2 Recognition Moiety is (N-(5-(1,1-dioxidoisothiazolidin-2-yl)-1H-indazol-3-yl)-2-(4-(piperidin-1-yl)phenyl)acetamide or a fragment or derivative thereof. For example, in certain embodiments, the CDK2 Recognition Moiety is selected from:

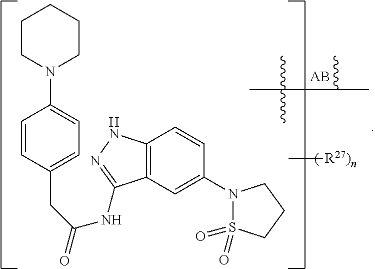

In certain embodiments the CDK2 Recognition Moiety is 4′-(3-bromo-7-((pyridin-3-ylmethyl)amino)pyrazolo[1,5-a]pyrimidin-5-yl)-[1,1′-biphenyl]-4-carboxamide or a fragment or derivative thereof. For example, in certain embodiments, the CDK2 Recognition Moiety is selected from:

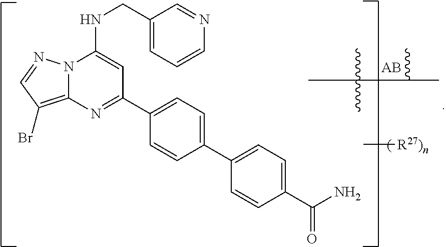

In certain embodiments the CDK2 Recognition Moiety is an RNA that binds CDK2. The RNA can be a fragment, SiRNA, a sequence of naturally occurring RNA, a sequence of unnatural RNA, or a combination thereof. In certain embodiments the RNA binds a viral target (see, for example, the paper by Bader Alhatlani, “In silico identification of conserved cis-acting RNA elements in the SARS COV-2 genome” Future Virology 15(7) 409-417). In another embodiment the RNA binds a protein that mediates a non-viral disorder such as a cancer or a tumor (see, for example, the paper by Xiangping Liang, et. al., “RNA-based pharmacotherapy for tumors: From bench to clinic and back” Biomedicine and Pharmacotherapy Volume 125, 2020, 109997).

In certain embodiments the CDK2 Recognition Moiety is a DNA that binds CDK2. The DNA can be a fragment, a sequence of naturally occurring DNA, a sequence of unnatural DNA, or a combination thereof (see, for example, the paper by Siddhesh D Patil, et al. “DNA-based therapeutics and DNA delivery systems: a comprehensive review” AAPS J. 2005 8; 7(1)).

In certain embodiments the CDK2 Recognition Moiety is

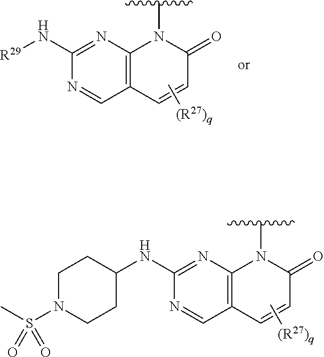

wherein

R²⁹ is independently selected at each instance from hydrogen, alkyl, haloalkyl, cycloalkyl, aryl, heterocycle, and heteroaryl each of which is optionally substituted with 1 or 2 substituents independently selected from R¹⁷ for example SO₂Me;

q is 0, 1, 2, or 3;

and wherein all other variables are as defined herein.

In certain embodiments q is 0. In certain embodiments q is 1.

In certain embodiments the CDK2 Recognition Moiety is

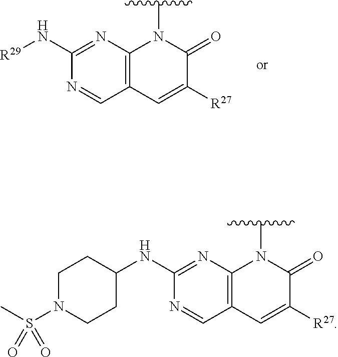

In certain embodiments the CDK2 Recognition Moiety is

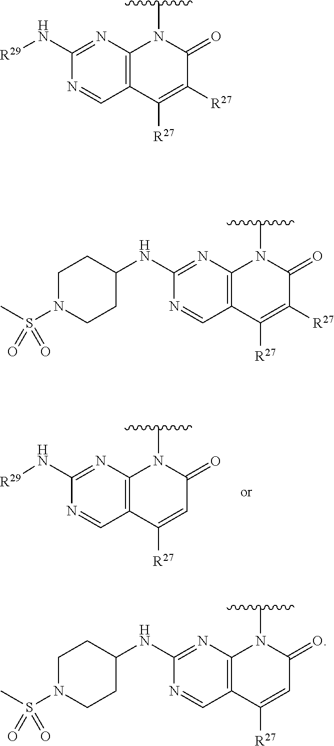

In certain embodiments CDK2 Recognition Moiety is selected from

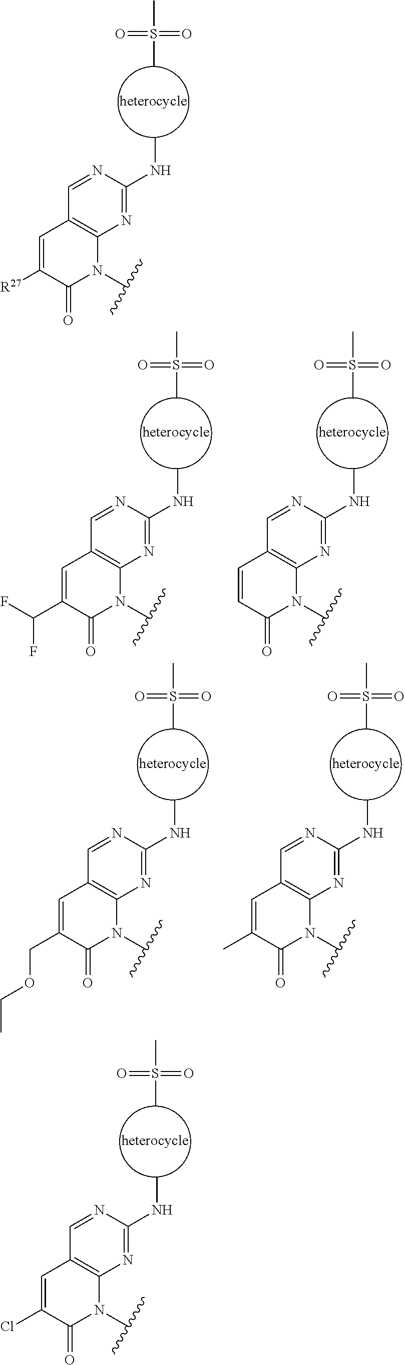

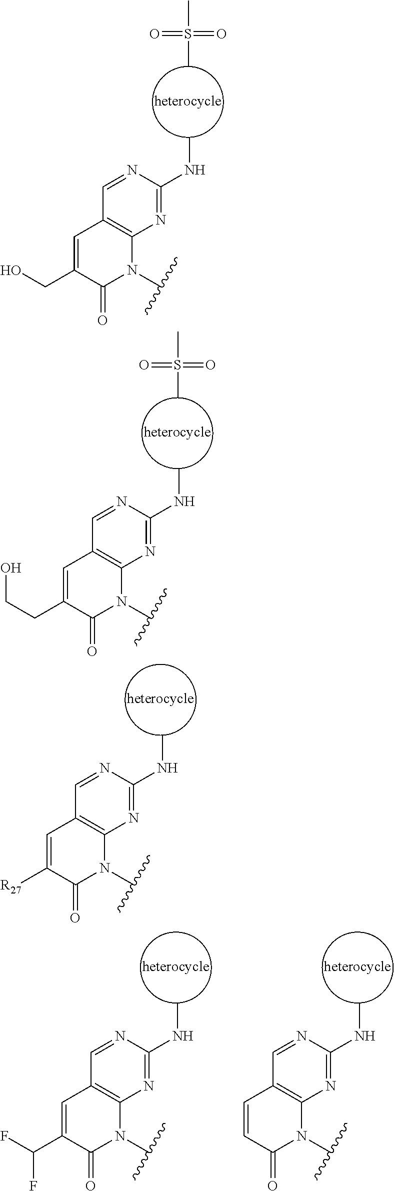

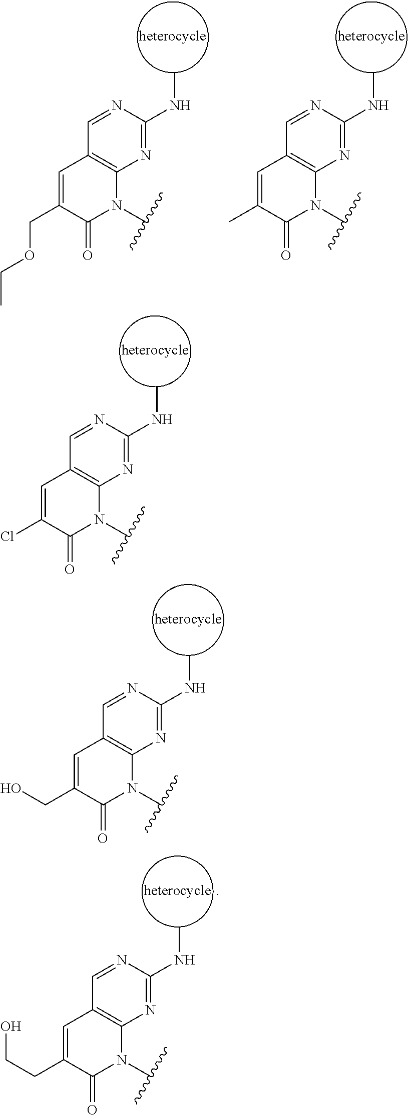

In certain embodiments CDK2 Recognition Moiety is selected from

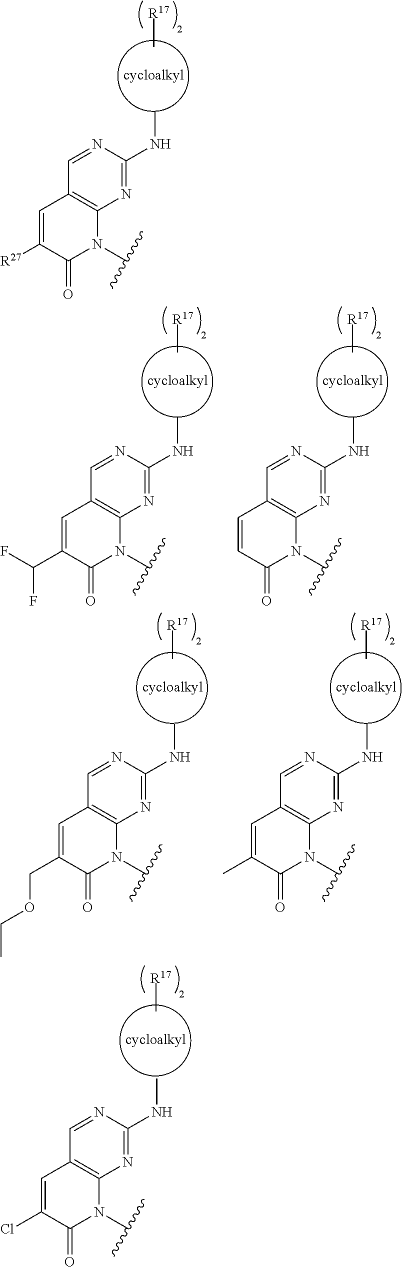

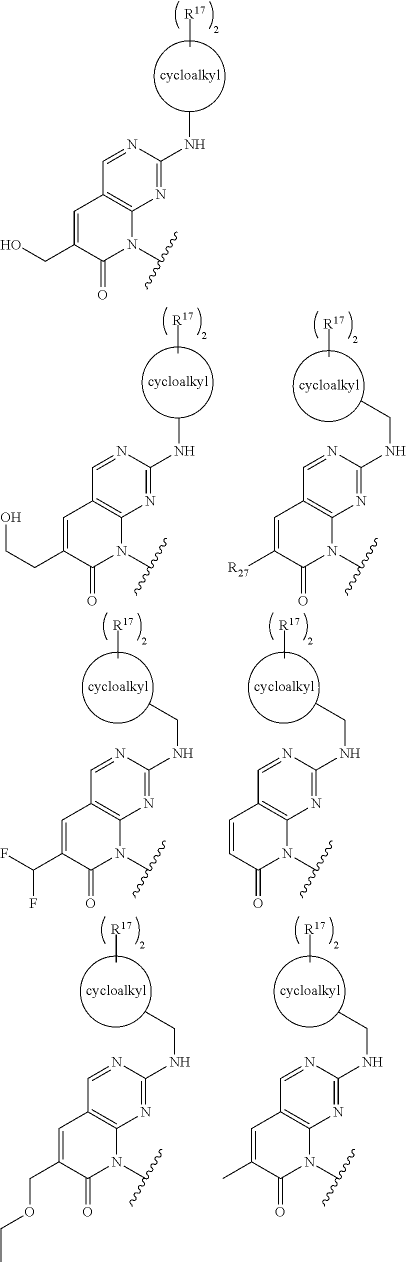

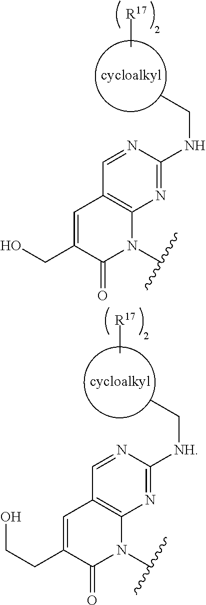

In certain embodiments CDK2 Recognition Moiety is selected from

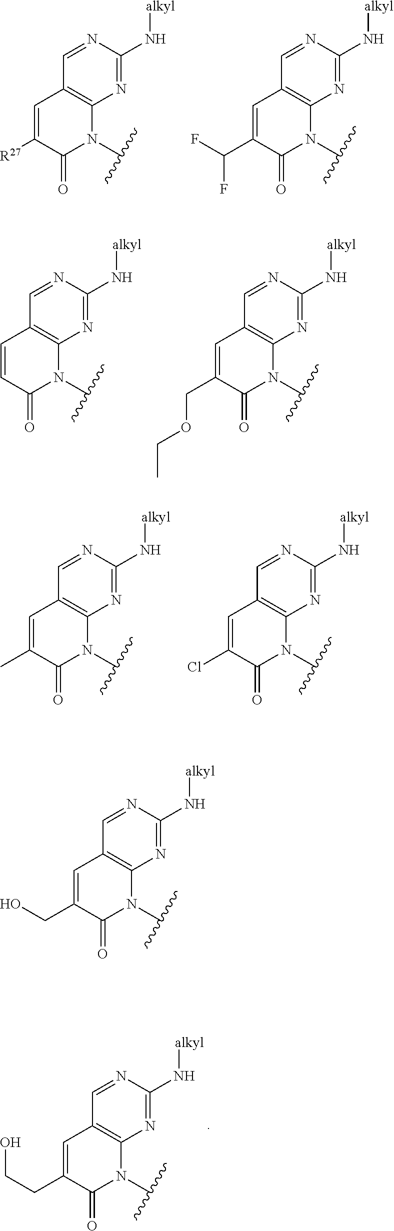

In certain embodiments CDK2 Recognition Moiety is

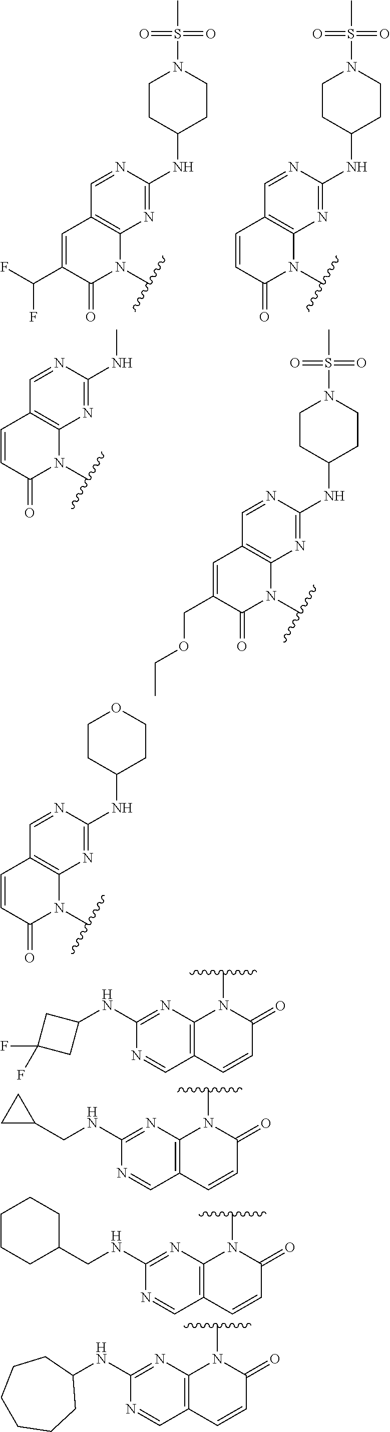

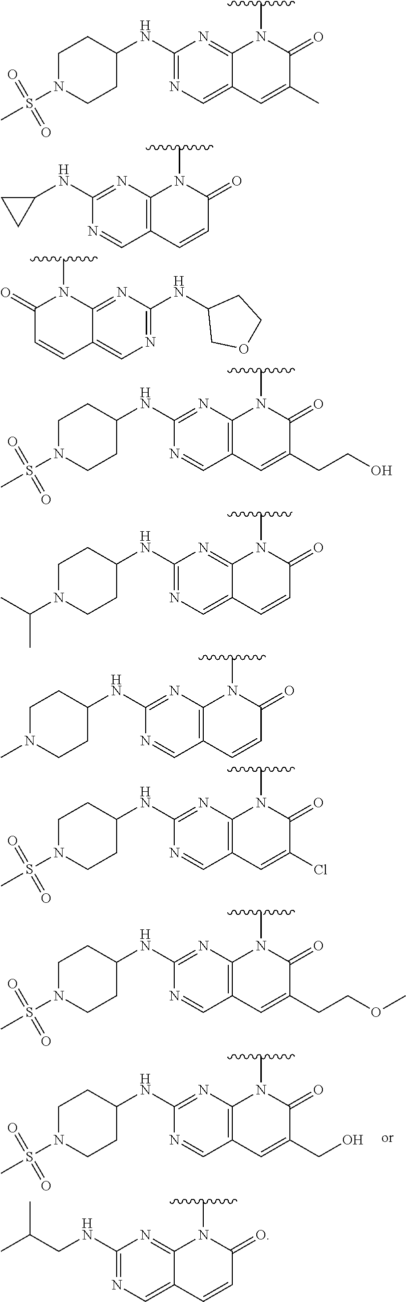

In certain embodiments the CDK2 Recognition Moiety binds fewer than 80, 70, 60, 50, 40, 30, 20, 15, 10, or 5 endogenous protein kinases with a KD50 of 10 μM or less.

In certain embodiments the CDK2 Recognition Moiety binds fewer than 80, 70, 60, 50, 40, 30, 20, 15, 10, or 5 endogenous protein kinases with a KD50 of 5 μM or less.

In certain embodiments the CDK2 Recognition Moiety binds fewer than 80, 70, 60, 50, 40, 30, 20, 15, 10, or 5 endogenous protein kinases with a KD50 of 2 μM or less.

In certain embodiments the CDK2 Recognition Moiety binds fewer than 80, 70, 60, 50, 40, 30, 20, 15, 10, or 5 endogenous protein kinases with a KD50 of 1 μM or less.

In certain embodiments the CDK2 Recognition Moiety binds fewer than 80, 70, 60, 50, 40, 30, 20, 15, 10, or 5 endogenous protein kinases with a KD50 of 0.5 μM or less.

### Exemplary Methods of Treatment of Diseases Mediated by CDK2

The present invention can be used to treat any disorder that is mediated by CDK2. Typically, the CDK2 Recognition Moiety is a targeting ligand or portion of a targeting ligand that binds or is bound by the CDK2 Recognition Moiety. Nonlimiting examples of disorders that can be treated with a heteroaryl sulfonyl compound of the present invention include abnormal cellular proliferation disorders such as a cancer or a tumor.

In certain embodiments a heteroaryl sulfonyl compound of the present invention is used to treat a leukemia, for example acute myeloid leukemia or relapsed/refractory chronic lymophocytic leukemia.

In certain embodiments a heteroaryl sulfonyl compound of the present invention is used to treat a myelodysplastic syndrome.

In certain embodiments a heteroaryl sulfonyl compound of the present invention is used to treat small cell lung cancer.

In certain embodiments a heteroaryl sulfonyl compound of the present invention is used to treat ovarian cancer.

In certain embodiments a heteroaryl sulfonyl compound of the present invention is used to treat triple negative breast cancer.

Non-limiting examples of cancers that can be treated according to the present invention include, but are not limited to, acoustic neuroma, adenocarcinoma, adrenal gland cancer, anal cancer, angiosarcoma (e.g., lymphangiosarcoma, lymphangioendotheliosarcoma, hemangiosarcoma), appendix cancer, benign monoclonal gammopathy, biliary cancer (e.g., cholangiocarcinoma), bladder cancer, breast cancer (e.g., adenocarcinoma of the breast, papillary carcinoma of the breast, mammary cancer, medullary carcinoma of the breast), brain cancer (e.g., meningioma; glioma, e.g., astrocytoma, oligodendroglioma; medulloblastoma), bronchus cancer, carcinoid tumor, cervical cancer (e.g., cervical adenocarcinoma), choriocarcinoma, chordoma, craniopharyngioma, colorectal cancer (e.g., colon cancer, rectal cancer, colorectal adenocarcinoma), epithelial carcinoma, ependymoma, endotheliosarcoma (e.g., Kaposi's sarcoma, multiple idiopathic hemorrhagic sarcoma), endometrial cancer (e.g., uterine cancer, uterine sarcoma), esophageal cancer (e.g., adenocarcinoma of the esophagus, Barrett's adenocarinoma), Ewing's sarcoma, eye cancer (e.g., intraocular melanoma, retinoblastoma), familiar hypereosinophilia, gall bladder cancer, gastric cancer (e.g., stomach adenocarcinoma), gastrointestinal stromal tumor (GIST), head and neck cancer (e.g., head and neck squamous cell carcinoma, oral cancer (e.g., oral squamous cell carcinoma (OSCC), throat cancer (e.g., laryngeal cancer, pharyngeal cancer, nasopharyngeal cancer, oropharyngeal cancer)), hematopoietic cancers (e.g., leukemia such as acute lymphocytic leukemia (ALL)—also known as acute lymphoblastic leukemia or acute lymphoid leukemia (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., “Waldenström's macroglobulinemia”), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma); a mixture of one or more leukemia/lymphoma as described above; and multiple myeloma (MM)), heavy chain disease (e.g., alpha chain disease, gamma chain disease, mu chain disease), hemangioblastoma, inflammatory myofibroblastic tumors, immunocytic amyloidosis, kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), leiomyosarcoma (LMS), mastocytosis (e.g., systemic mastocytosis), myelodysplastic syndrome (MDS), mesothelioma, myeloproliferative disorder (MPD) (e.g., polycythemia Vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)), neuroblastoma, neurofibroma (e.g., neurofibromatosis (NF) type 1 or type 2, schwannomatosis), neuroendocrine cancer (e.g., gastroenteropancreatic neuroendoctrine tumor (GEP-NET), carcinoid tumor), osteosarcoma, ovarian cancer (e.g., cystadenocarcinoma, ovarian embryonal carcinoma, ovarian adenocarcinoma), papillary adenocarcinoma, pancreatic cancer (e.g., pancreatic andenocarcinoma, intraductal papillary mucinous neoplasm (IPMN), Islet cell tumors), penile cancer (e.g., Paget's disease of the penis and scrotum), pinealoma, primitive neuroectodermal tumor (PNT), prostate cancer (e.g., prostate adenocarcinoma), rectal cancer, rhabdomyosarcoma, salivary gland cancer, skin cancer (e.g., squamous cell carcinoma (SCC), keratoacanthoma (KA), melanoma, basal cell carcinoma (BCC)), small bowel cancer (e.g., appendix cancer), soft tissue sarcoma (e.g., malignant fibrous histiocytoma (MFH), liposarcoma, malignant peripheral nerve sheath tumor (MPNST), chondrosarcoma, fibrosarcoma, myxosarcoma), sebaceous gland carcinoma, sweat gland carcinoma, synovioma, testicular cancer (e.g., seminoma, testicular embryonal carcinoma), thyroid cancer (e.g., papillary carcinoma of the thyroid, papillary thyroid carcinoma (PTC), medullary thyroid cancer), urethral cancer, vaginal cancer and vulvar cancer (e.g., Paget's disease of the vulva).

In certain embodiments, the cancer is a hematopoietic cancer. In certain embodiments, the hematopoietic cancer is a lymphoma. In certain embodiments, the hematopoietic cancer is a leukemia. In certain embodiments, the leukemia is acute myelocytic leukemia (AML).

In certain embodiments, the proliferative disorder is a myeloproliferative neoplasm. In certain embodiments, the myeloproliferative neoplasm (MPN) is primary myelofibrosis (PMF).

In certain embodiments, the cancer is a solid tumor. A solid tumor, as used herein, refers to an abnormal mass of tissue that usually does not contain cysts or liquid areas. Different types of solid tumors are named for the type of cells that form them. Examples of classes of solid tumors include, but are not limited to, sarcomas, carcinomas, and lymphomas, as described above herein. Additional examples of solid tumors include, but are not limited to, squamous cell carcinoma, colon cancer, breast cancer, prostate cancer, lung cancer, liver cancer, pancreatic cancer, and melanoma) (Wadler, Scott, “Perspectives for cancer therapies with cdk2 inhibitors”, Drug Resistance Updates, 2001, 4(6), 347-367; https://www.mycancergenome.org/content/gene/cdk2/; Tavakolian, S., Goudarzi, H. & Faghihloo, E. Cyclin-dependent kinases and CDK inhibitors in virus-associated cancers. Infect Agents Cancer, 2020, 15, 27).

Abnormal cellular proliferation, notably hyperproliferation, can occur as a result of a wide variety of factors, including genetic mutation, infection, exposure to toxins, autoimmune disorders, and benign or malignant tumor induction.

There are a number of skin disorders associated with cellular hyperproliferation. Psoriasis, for example, is a benign disease of human skin generally characterized by plaques covered by thickened scales. The disease is caused by increased proliferation of epidermal cells of unknown cause. Chronic eczema is also associated with significant hyperproliferation of the epidermis. Other diseases caused by hyperproliferation of skin cells include atopic dermatitis, lichen planus, warts, pemphigus vulgaris, actinic keratosis, basal cell carcinoma and squamous cell carcinoma (Lanza et al., “Evidence of key role of Cdk2 overexpression in pemphigus vulgaris”, J Biol Chem 2008 Mar. 28; 283(13):8736-45).

Other hyperproliferative cell disorders include blood vessel proliferation disorders, fibrotic disorders, autoimmune disorders, graft-versus-host rejection, tumors and cancers.

Blood vessel proliferative disorders include angiogenic and vasculogenic disorders. Proliferation of smooth muscle cells in the course of development of plaques in vascular tissue cause, for example, restenosis, retinopathies and atherosclerosis. Both cell migration and cell proliferation play a role in the formation of atherosclerotic lesions.

Fibrotic disorders are often due to the abnormal formation of an extracellular matrix. Examples of fibrotic disorders include hepatic cirrhosis and mesangial proliferative cell disorders. Hepatic cirrhosis is characterized by the increase in extracellular matrix constituents resulting in the formation of a hepatic scar. Hepatic cirrhosis can cause diseases such as cirrhosis of the liver. An increased extracellular matrix resulting in a hepatic scar can also be caused by viral infection such as hepatitis. Lipocytes appear to play a major role in hepatic cirrhosis.

Mesangial disorders are brought about by abnormal proliferation of mesangial cells. Mesangial hyperproliferative cell disorders include various human renal diseases, such as glomerulonephritis, diabetic nephropathy, malignant nephrosclerosis, thrombotic micro-angiopathy syndromes, transplant rejection, and glomerulopathies.

Another disease with a proliferative component is rheumatoid arthritis. Rheumatoid arthritis is generally considered an autoimmune disease that is thought to be associated with activity of autoreactive T cells, and to be caused by autoantibodies produced against collagen and IgE.

Other disorders that can include an abnormal cellular proliferative component include Bechet's syndrome, acute respiratory distress syndrome (ARDS), ischemic heart disease, post-dialysis syndrome, leukemia, acquired immune deficiency syndrome, vasculitis, lipid histiocytosis, septic shock and inflammation in general.

Exemplary cancers which may be treated by the disclosed heteroaryl sulfonyl compounds either alone or in combination with at least one additional anti-cancer agent include squamous-cell carcinoma, basal cell carcinoma, adenocarcinoma, hepatocellular carcinomas, and renal cell carcinomas, cancer of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's lymphoma; benign and malignant melanomas; myeloproliferative diseases; sarcomas, including Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas; bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, melanoma; carcinosarcoma, Hodgkin's disease, Wilms' tumor and teratocarcinomas. Additional cancers which may be treated using the disclosed heteroaryl sulfonyl compounds according to the present invention include, for example, acute granulocytic leukemia, acute lymphocytic leukemia (ALL), acute myelogenous leukemia (AML), adenocarcinoma, adenosarcoma, adrenal cancer, adrenocortical carcinoma, anal cancer, anaplastic astrocytoma, angiosarcoma, appendix cancer, astrocytoma, Basal cell carcinoma, B-Cell lymphoma, bile duct cancer, bladder cancer, bone cancer, bone marrow cancer, bowel cancer, brain cancer, brain stem glioma, breast cancer, triple (estrogen, progesterone and HER-2) negative breast cancer, double negative breast cancer (two of estrogen, progesterone and HER-2 are negative), single negative (one of estrogen, progesterone and HER-2 is negative), estrogen-receptor positive, HER2-negative breast cancer, estrogen receptor-negative breast cancer, estrogen receptor positive breast cancer, metastatic breast cancer, luminal A breast cancer, luminal B breast cancer, Her2-negative breast cancer, HER2-positive or negative breast cancer, progesterone receptor-negative breast cancer, progesterone receptor-positive breast cancer, recurrent breast cancer, carcinoid tumors, cervical cancer, cholangiocarcinoma, chondrosarcoma, chronic lymphocytic leukemia (CLL), chronic myelogenous leukemia (CML), colon cancer, colorectal cancer, craniopharyngioma, cutaneous lymphoma, cutaneous melanoma, diffuse astrocytoma, ductal carcinoma in situ (DCIS), endometrial cancer, ependymoma, epithelioid sarcoma, esophageal cancer, ewing sarcoma, extrahepatic bile duct cancer, eye cancer, fallopian tube cancer, fibrosarcoma, gallbladder cancer, gastric cancer, gastrointestinal cancer, gastrointestinal carcinoid cancer, gastrointestinal stromal tumors (GIST), germ cell tumor glioblastoma multiforme (GBM), glioblastoma, recurrent glioblastoma, glioma, hairy cell leukemia, head and neck cancer, hemangioendothelioma, Hodgkin lymphoma, hypopharyngeal cancer, infiltrating ductal carcinoma (IDC), infiltrating lobular carcinoma (ILC), inflammatory breast cancer (IBC), intestinal Cancer, intrahepatic bile duct cancer, invasive/infiltrating breast cancer, Islet cell cancer, jaw cancer, Kaposi sarcoma, kidney cancer, laryngeal cancer, leiomyosarcoma, leptomeningeal metastases, leukemia, lip cancer, liposarcoma, liver cancer, lobular carcinoma in situ, low-grade astrocytoma, lung cancer, lymph node cancer, lymphoma, male breast cancer, medullary carcinoma, medulloblastoma, melanoma, meningioma, Merkel cell carcinoma, mesenchymal chondrosarcoma, mesenchymous, mesothelioma metastatic breast cancer, metastatic melanoma metastatic squamous neck cancer, mixed gliomas, monodermal teratoma, mouth cancer mucinous carcinoma, mucosal melanoma, multiple myeloma, Mycosis Fungoides, myelodysplastic syndrome, nasal cavity cancer, nasopharyngeal cancer, neck cancer, neuroblastoma, neuroendocrine tumors (NETs), non-Hodgkin's lymphoma, non-small cell lung cancer (NSCLC), oat cell cancer, ocular cancer, ocular melanoma, oligodendroglioma, oral cancer, oral cavity cancer, oropharyngeal cancer, osteogenic sarcoma, osteosarcoma, ovarian cancer, ovarian epithelial cancer ovarian germ cell tumor, ovarian primary peritoneal carcinoma, ovarian sex cord stromal tumor, Paget's disease, pancreatic cancer, papillary carcinoma, paranasal sinus cancer, parathyroid cancer, pelvic cancer, penile cancer, peripheral nerve cancer, peritoneal cancer, pharyngeal cancer, pheochromocytoma, pilocytic astrocytoma, pineal region tumor, pineoblastoma, pituitary gland cancer, primary central nervous system (CNS) lymphoma, prostate cancer, rectal cancer, renal cell carcinoma, renal pelvis cancer, rhabdomyosarcoma, salivary gland cancer, soft tissue sarcoma, bone sarcoma, sarcoma, sinus cancer, skin cancer, small cell lung cancer (SCLC), small intestine cancer, spinal cancer, spinal column cancer, spinal cord cancer, squamous cell carcinoma, stomach cancer, synovial sarcoma, T-cell lymphoma, testicular cancer, throat cancer, thymoma/thymic carcinoma, thyroid cancer, tongue cancer, tonsil cancer, transitional cell cancer, tubal cancer, tubular carcinoma, undiagnosed cancer, ureteral cancer, urethral cancer, uterine adenocarcinoma, uterine cancer, uterine sarcoma, vaginal cancer, vulvar cancer, T-cell lineage acute lymphoblastic leukemia (T-ALL), T-cell lineage lymphoblastic lymphoma (T-LL), peripheral T-cell lymphoma, Adult T-cell leukemia, Pre-B ALL, Pre-B lymphomas, large B-cell lymphoma, Burkitts lymphoma, B-cell ALL, Philadelphia chromosome positive ALL, Philadelphia chromosome positive CML, juvenile myelomonocytic leukemia (JMIML), acute promyelocytic leukemia (a subtype of AML), large granular lymphocytic leukemia, Adult T-cell chronic leukemia, diffuse large B cell lymphoma, follicular lymphoma; Mucosa-Associated Lymphatic Tissue lymphoma (MALT), small cell lymphocytic lymphoma, mediastinal large B cell lymphoma, nodal marginal zone B cell lymphoma (NMZL); splenic marginal zone lymphoma (SMZL); intravascular large B-cell lymphoma; primary effusion lymphoma; or lymphomatoid granulomatosis; B-cell prolymphocytic leukemia; splenic lymphoma/leukemia, unclassifiable, splenic diffuse red pulp small B-cell lymphoma; lymphoplasmacytic lymphoma; heavy chain diseases, for example, Alpha heavy chain disease, Gamma heavy chain disease, Mu heavy chain disease, plasma cell myeloma, solitary plasmacytoma of bone; extraosseous plasmacytoma; primary cutaneous follicle center lymphoma, T cell/histocyte rich large B-cell lymphoma, DLBCL associated with chronic inflammation; Epstein-Barr virus (EBV)+DLBCL of the elderly; primary mediastinal (thymic) large B-cell lymphoma, primary cutaneous DLBCL, leg type, ALK+ large B-cell lymphoma, plasmablastic lymphoma; large B-cell lymphoma arising in HHV8-associated multicentric, Castleman disease; B-cell lymphoma, unclassifiable, with features intermediate between diffuse large B-cell lymphoma, or B-cell lymphoma, unclassifiable, with features intermediate between diffuse large B-cell lymphoma and classical Hodgkin lymphoma (Yu et al., “DNA damage induces cdk2 protein levels and histone H2B phosphorylation in SH-SY5Y neuroblastoma cells”, J Alzheimer's Dis., 2005 September; 8(1):7-21).

Other exemplary diseases which may be treated by the disclosed heteroaryl sulfonyl compounds either alone or in combination with at least one additional pharmaceutical agent include ocular disease, diabetes, kidney disease, liver disease, cancer-associated viruses (for example, Human papillomaviruses (HPV), Human T-lymphotropic viruses (HTLV), Human endogenous retroviruses (HERV), Epstein-Barr virus (EBV), Herpes virus (KSHV), Human hepatitis B virus (HBV), Hepatitis C virus (HCV)), autoimmune diseases (for example, Pemphigus vulgaris (PV), rheumatic arthritis, diabetes), neutrophil-mediated inflammation, and neurodegenerative disease (for example, Alzheimer disease, Parkinson's disease, and amyotrophic lateral sclerosis).

### Pharmaceutical Compositions

A heteroaryl sulfonyl compound of the present invention or a pharmaceutically acceptable salt, solvate or prodrug thereof as disclosed herein can be administered as a neat chemical, but is more typically administered as a pharmaceutical composition that includes an effective amount for a host, typically a human, in need of such treatment to treat a disorder mediated by the target extracellular protein, as described herein or otherwise well-known for that extracellular protein.

The heteroaryl sulfonyl compounds of the present invention can be administered in any manner that allows the heteroaryl sulfonyl compound to covalently modify CDK2. As such, examples of methods to deliver a heteroaryl sulfonyl compound of the present invention include, but are not limited to, oral, intravenous, sublingual, subcutaneous, parenteral, buccal, rectal, intra-aortal, intracranial, subdermal, transdermal, controlled drug delivery, intramuscular, or transnasal, or by other means, in dosage unit formulations containing one or more conventional pharmaceutically acceptable carriers, as appropriate. In certain embodiments, a heteroaryl sulfonyl compound of the present invention is provided in a liquid dosage form, a solid dosage form, a gel, particle, etc.

In certain embodiments the heteroaryl sulfonyl compound of the present invention is administered subcutaneously. Typically, the heteroaryl sulfonyl compound will be formulated in a liquid dosage form for subcutaneous injection, such as a buffered solution. Non-limiting examples of solutions for subcutaneous injection include phosphate buffered solution and saline buffered solution. In certain embodiments the solution is buffered with multiple salts.

In certain embodiments the heteroaryl sulfonyl compound of the present invention is administered intravenously. Typically, if administered intravenously, the heteroaryl sulfonyl compound will be formulated in a liquid dosage form for intravenous injection, such as a buffered solution. Non-limiting examples of solutions for intravenous injection include phosphate buffered solution and saline buffered solution. In certain embodiments the solution is buffered with multiple salts.

Therefore, the disclosure provides pharmaceutical compositions comprising an effective amount of heteroaryl sulfonyl compound or its pharmaceutically acceptable salt together with at least one pharmaceutically acceptable carrier for any appropriate use thereof. The pharmaceutical composition may contain a heteroaryl sulfonyl compound or salt as the only active agent, or, in an alternative embodiment, the heteroaryl sulfonyl compound and at least one additional active agent.

The term “pharmaceutically acceptable salt” as used herein refers to a salt of the described heteroaryl sulfonyl compound which is, within the scope of sound medical judgment, suitable for administration to a host such as a human without undue toxicity, irritation, allergic response, and the like, commensurate with a reasonable benefit/risk ratio, and effective for its intended use. Thus, the term “pharmaceutically acceptable salt” refers to the relatively non-toxic, inorganic and organic acid addition salts of the presently disclosed heteroaryl sulfonyl compounds. These salts can be prepared during the final isolation and purification of the heteroaryl sulfonyl compounds or by separately reacting the purified heteroaryl sulfonyl compound in its free form with a suitable organic or inorganic acid and then isolating the salt thus formed. Basic heteroaryl sulfonyl compounds are capable of forming a wide variety of different salts with various inorganic and organic acids. Acid addition salts of the basic heteroaryl sulfonyl compounds are prepared by contacting the free base form with a sufficient amount of the desired acid to produce the salt in the conventional manner. The free base form can be regenerated by contacting the salt form with a base and isolating the free base in the conventional manner. The free base forms may differ from their respective salt forms in certain physical properties such as solubility in polar solvents.

Pharmaceutically acceptable base addition salts may be formed with a metal or amine, such as alkali and alkaline earth metal hydroxide, or an organic amine. Examples of metals used as cations, include, but are not limited to, sodium, potassium, magnesium, calcium, and the like. Examples of suitable amines include, but are not limited to, N,N′-dibenzylethylenediamine, chloroprocaine, choline, diethanolamine, ethylenediamine, N-methylglucamine, and procaine. The base addition salts of acidic heteroaryl sulfonyl compounds are prepared by contacting the free acid form with a sufficient amount of the desired base to produce the salt in the conventional manner. The free acid form can be regenerated by contacting the salt form with an acid and isolating the free acid in a conventional manner. The free acid forms may differ from their respective salt forms somewhat in certain physical properties such as solubility in polar solvents.

Salts can be prepared from inorganic acids sulfate, pyrosulfate, bisulfate, sulfite, bisulfite, nitrate, phosphate, monohydrogenphosphate, dihydrogenphosphate, metaphosphate, pyrophosphate, chloride, bromide, iodide, nitric, phosphoric, sulfuric, hydrobromic, hydriodic, phosphorus, and the like. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, laurylsulphonate and isethionate salts, and the like. Salts can also be prepared from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. and the like. Representative salts include acetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Pharmaceutically acceptable salts can include cations based on the alkali and alkaline earth metals, such as sodium, lithium, potassium, calcium, magnesium and the like, as well as non-toxic ammonium, quaternary ammonium, and amine cations including, but not limited to, ammonium, tetramethylammonium, tetraethylammonium, methylamine, dimethylamine, trimethylamine, triethylamine, ethylamine, and the like. Also contemplated are the salts of amino acids such as arginate, gluconate, galacturonate, and the like. See, for example, Berge et al., J. Pharm. Sci., 1977, 66, 1-19, which is incorporated herein by reference.

Any dosage form can be used that achieves the desired results. In certain embodiments the pharmaceutical composition is in a dosage form that contains from about 0.1 mg to about 1500 mg, from about 10 mg to about 1000 mg, from about 100 mg to about 800 mg, or from about 200 mg to about 600 mg of the active heteroaryl sulfonyl compound and optionally from about 0.1 mg to about 1500 mg, from about 10 mg to about 1000 mg, from about 100 mg to about 800 mg, or from about 200 mg to about 600 mg of an additional active agent in a unit dosage form. Examples are dosage forms with at least 0.1, 1, 5, 10, 25, 50, 100, 200, 250, 300, 400, 500, 600, 700, or 750 mg of active heteroaryl sulfonyl compound, or its salt.

In certain embodiments the dose ranges from about 0.01-100 mg/kg of patient bodyweight, for example about 0.01 mg/kg, about 0.05 mg/kg, about 0.1 mg/kg, about 0.5 mg/kg, about 1 mg/kg, about 1.5 mg/kg, about 2 mg/kg, about 2.5 mg/kg, about 3 mg/kg, about 3.5 mg/kg, about 4 mg/kg, about 4.5 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, or about 100 mg/kg.

In some embodiments, heteroaryl sulfonyl compounds disclosed herein or used as described are administered once a day (QD), twice a day (BID), or three times a day (TID). In some embodiments, heteroaryl sulfonyl compounds disclosed herein or used as described are administered at least once a day for at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, at least 31 days, at least 35 days, at least 45 days, at least 60 days, at least 75 days, at least 90 days, at least 120 days, at least 150 days, at least 180 days, or longer.

In certain embodiments the heteroaryl sulfonyl compound of the present invention is administered once a day, twice a day, three times a day, or four times a day.

The pharmaceutical composition may be formulated as any pharmaceutically useful form, e.g., a pill, capsule, tablet, an injection or infusion solution, a syrup, an inhalation formulation, a suppository, a buccal or sublingual formulation, a parenteral formulation, or in a medical device.

Some dosage forms, such as tablets and capsules, can be subdivided into suitably sized unit doses containing appropriate quantities of the active components, e.g., an effective amount to achieve the desired purpose.

Carriers include excipients and diluents and must be of sufficiently high purity and sufficiently low toxicity to render them suitable for administration to the patient being treated. The carrier can be inert or it can possess pharmaceutical benefits of its own. The amount of carrier employed in conjunction with the heteroaryl sulfonyl compound is sufficient to provide a practical quantity of material for administration per unit dose of the heteroaryl sulfonyl compound. If provided as in a liquid, it can be a solution or a suspension.

Representative carriers include phosphate buffered saline, water, solvent(s), diluents, pH modifying agents, preservatives, antioxidants, suspending agents, wetting agent, viscosity agents, tonicity agents, stabilizing agents, and combinations thereof. In some embodiments, the carrier is an aqueous carrier. Examples of aqueous carries include, but are not limited to, an aqueous solution or suspension, such as saline, plasma, bone marrow aspirate, buffers, such as Hank's Buffered Salt Solution (HBSS), HEPES (4-(2-hydroxyethyl)-1-piperazineethanesulfonic acid), Ringers buffer, ProVisc®, diluted ProVisc®, Provisc® diluted with PBS, Krebs buffer, Dulbecco's PBS, normal PBS, sodium hyaluronate solution (HA, 5 mg/mL in PBS), citrate buffer, simulated body fluids, plasma platelet concentrate and tissue culture medium or an aqueous solution or suspension comprising an organic solvent. Acceptable solutions include, for example, water, Ringer's solution and isotonic sodium chloride solutions. The formulation may also be a sterile solution, suspension, or emulsion in a non-toxic diluent or solvent such as 1,3-butanediol.

Viscosity agents may be added to the pharmaceutical composition to increase the viscosity of the composition as desired. Examples of useful viscosity agents include, but are not limited to, hyaluronic acid, sodium hyaluronate, carbomers, polyacrylic acid, cellulosic derivatives, polycarbophil, polyvinylpyrrolidone, gelatin, dextin, polysaccharides, polyacrylamide, polyvinyl alcohol (including partially hydrolyzed polyvinyl acetate), polyvinyl acetate, derivatives thereof and mixtures thereof.

Solutions, suspensions, or emulsions for administration may be buffered with an effective amount necessary to maintain a pH suitable for the selected administration. Suitable buffers are well known by those skilled in the art. Some examples of useful buffers are acetate, borate, carbonate, citrate, and phosphate buffers. Solutions, suspensions, or emulsions for topical, for example, ocular administration may also contain one or more tonicity agents to adjust the isotonic range of the formulation. Suitable tonicity agents are well known in the art. Some examples include glycerin, mannitol, sorbitol, sodium chloride, and other electrolytes.

Classes of carriers include, but are not limited to binders, buffering agents, coloring agents, diluents, disintegrants, emulsifiers, flavorants, glidants, lubricants, preservatives, stabilizers, surfactants, tableting agents, and wetting agents. Some carriers may be listed in more than one class, for example vegetable oil may be used as a lubricant in some formulations and a diluent in others. Exemplary pharmaceutically acceptable carriers include sugars, starches, celluloses, powdered tragacanth, malt, gelatin; talc, and vegetable oils. Optional active agents may be included in a pharmaceutical composition, which do not substantially interfere with the activity of the heteroaryl sulfonyl compound of the present invention.

The pharmaceutical compositions/combinations can be formulated for oral administration. These compositions can contain any amount of active heteroaryl sulfonyl compound that achieves the desired result, for example between 0.1 and 99 weight % (wt. %) of the heteroaryl sulfonyl compound and usually at least about 1 wt. % of the heteroaryl sulfonyl compound. Some embodiments contain from about 25 wt. % to about 50 wt. % or from about 5 wt. % to about 75 wt. % of the heteroaryl sulfonyl compound. Enteric coated oral tablets may also be used to enhance bioavailability of the heteroaryl sulfonyl compounds for an oral route of administration.

Formulations suitable for rectal administration are typically presented as unit dose suppositories. These may be prepared by admixing the active heteroaryl sulfonyl compound with one or more conventional solid carriers, for example, cocoa butter, and then shaping the resulting mixture.

### Processes of Manufacture:

The heteroaryl sulfonyl compounds of the present invention can be manufactured according to routes described in the Working Examples below or as otherwise known in the patent or scientific literature and if appropriate supported by the knowledge of the ordinary worker or common general knowledge.

Some of the carbons in the heteroaryl sulfonyl compounds described herein are drawn with designated stereochemistry. Other carbons are drawn without stereochemical designation. When drawn without designated stereochemistry, that carbon can be in any desired stereochemical configuration that achieves the desired purpose. One skilled in the art will recognize that pure enantiomers, enantiomerically enriched heteroaryl sulfonyl compounds, racemates and diastereomers can be prepared by methods known in the art as guided by the information provided herein. Examples of methods to obtain optically active materials include at least the following:
i) chiral liquid chromatography—a technique whereby diastereomers are separated in a liquid mobile phase by virtue of their differing interactions with a stationary phase (including vial chiral HPLC). The stationary phase can be made of chiral material or the mobile phase can contain an additional chiral material to provoke the differing interactions;
ii) non-chiral chromatography of diastereomers-Often diastereomers can be separated using normal non-chiral column conditions;
iii) chiral gas chromatography—a technique whereby the racemate is volatilized and enantiomers are separated by virtue of their differing interactions in the gaseous mobile phase with a column containing a fixed non-racemic chiral adsorbent phase;
iv) simultaneous crystallization—a technique whereby the individual diastereomers are separately crystallized from a solution;
v) enzymatic resolutions—a technique whereby partial or complete separation of diastereomers are separated by virtue of differing rates of reaction with an enzyme;
vi) chemical asymmetric synthesis—a synthetic technique whereby the desired diastereomer is synthesized from an achiral precursor under conditions that produce asymmetry (i.e. chirality) in the product, which may be achieved by chiral catalysts or chiral auxiliaries;
vii) diastereomer separations—a technique whereby a racemic heteroaryl sulfonyl compound is reacted with an enantiomerically pure reagent (the chiral auxiliary) that converts the individual enantiomers to diastereomers. The resulting diastereomers are then separated by chromatography or crystallization by virtue of their now more distinct structural differences the chiral auxiliary later removed to obtain the desired enantiomer; and
viii) extraction with chiral solvents—a technique whereby diastereomers are separated by virtue of preferential dissolution of one over the others in a particular chiral solvent.

### Synthesis of Compound 101

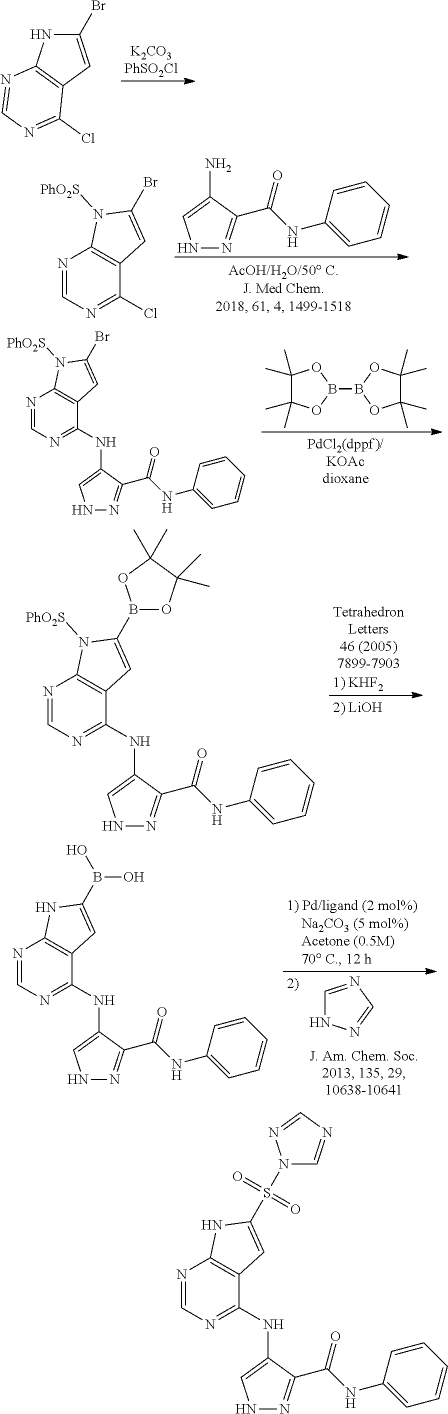

### Synthesis of Compound 102

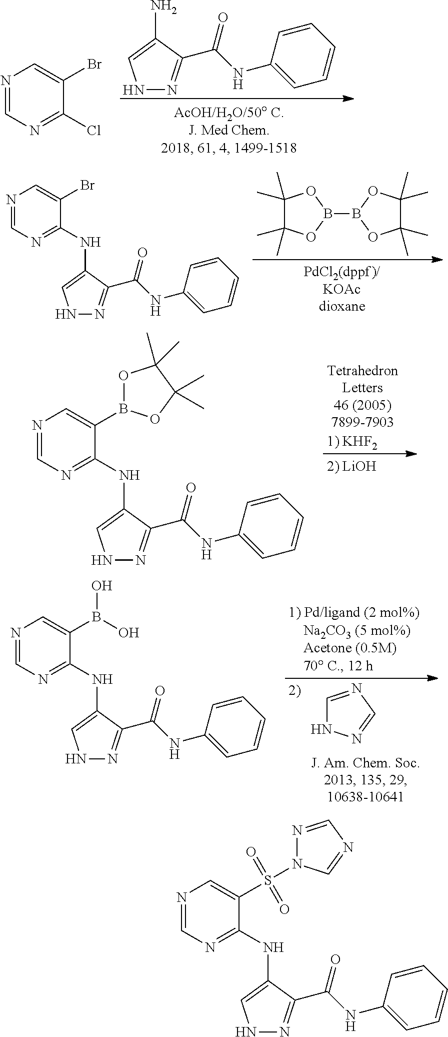

### Synthesis of Compound 103

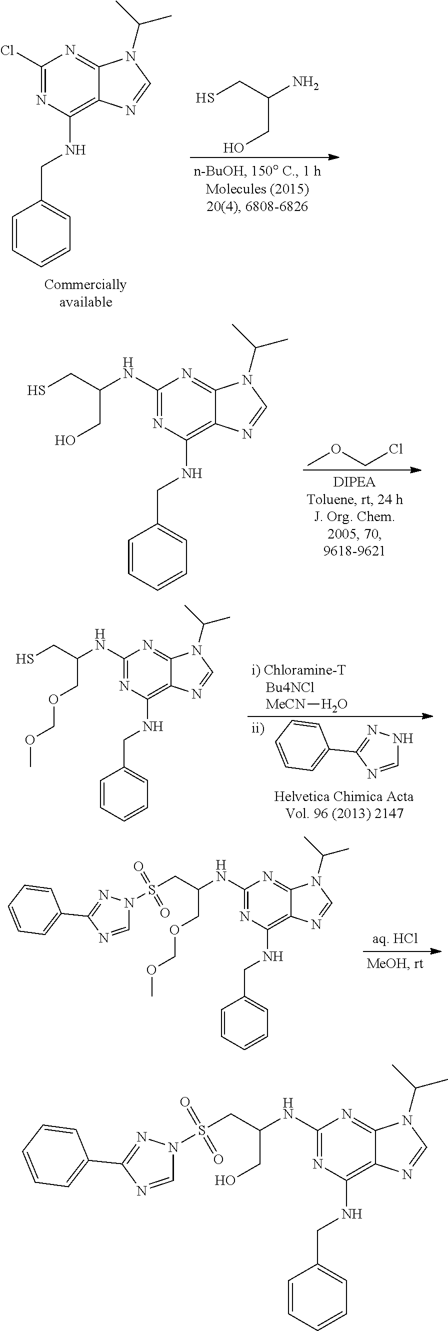

### Synthesis of Compound 104

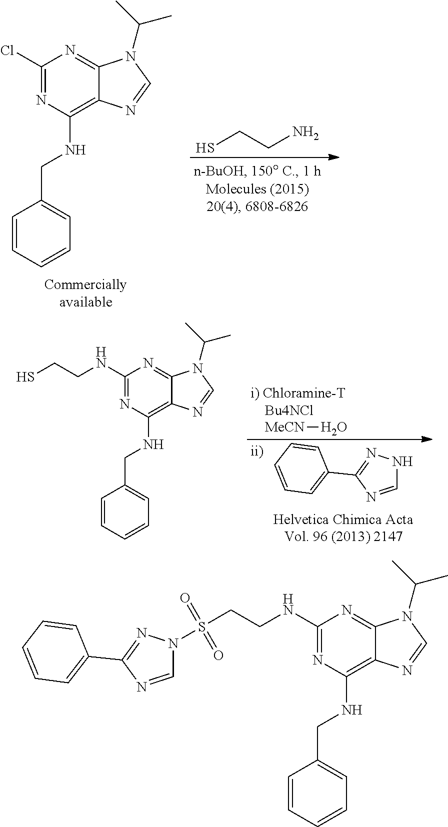

### Synthesis of Compound 105

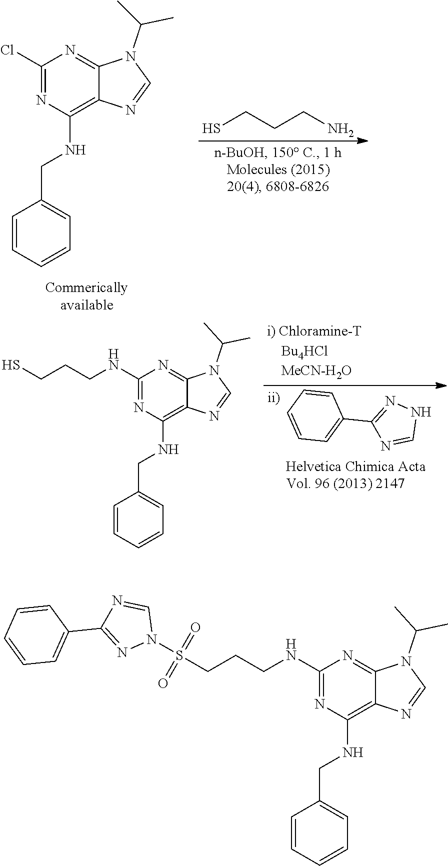 [SMILES: CC(C)n1cnc2c(NCc3ccccc3)nc(Cl)nc21 ; NCCCS ; CC(C)n1cnc2c(NCc3ccccc3)nc(NCCCS)nc21 ; c1ccc(-c2nc[nH]n2)cc1 ; CC(C)n1cnc2c(NCc3ccccc3)nc(NCCCS(=O)(=O)n3cnc(-c4ccccc4)n3)nc21]

### Synthesis of Compound 106

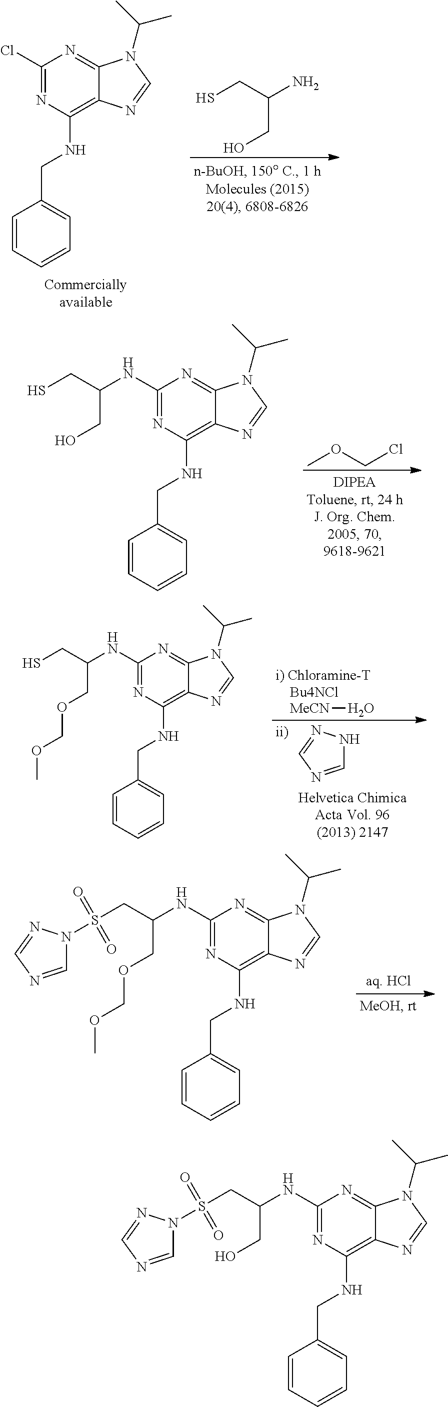

### Synthesis of Compound 107

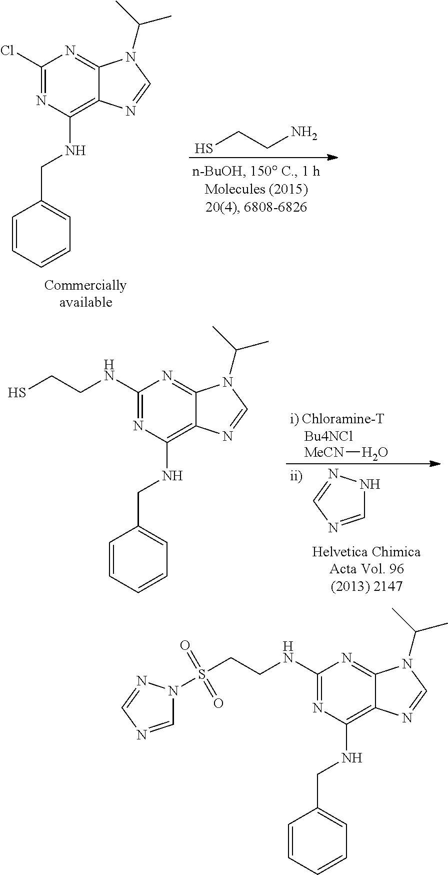

### Synthesis of Compound 108

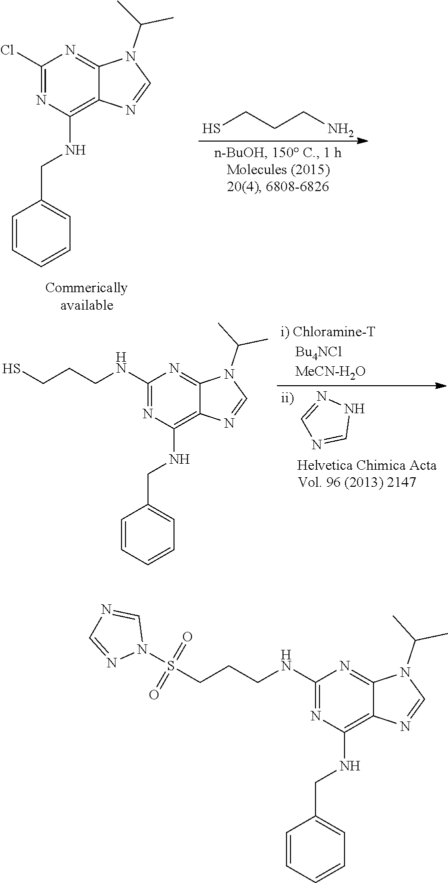 [SMILES: CC(C)n1cnc2c(NCc3ccccc3)nc(Cl)nc21 ; NCCCS ; CC(C)n1cnc2c(NCc3ccccc3)nc(NCCCS)nc21 ; c1nc[nH]n1 ; CC(C)n1cnc2c(NCc3ccccc3)nc(NCCCS(=O)(=O)n3cncn3)nc21]

### Synthesis of Compound 109

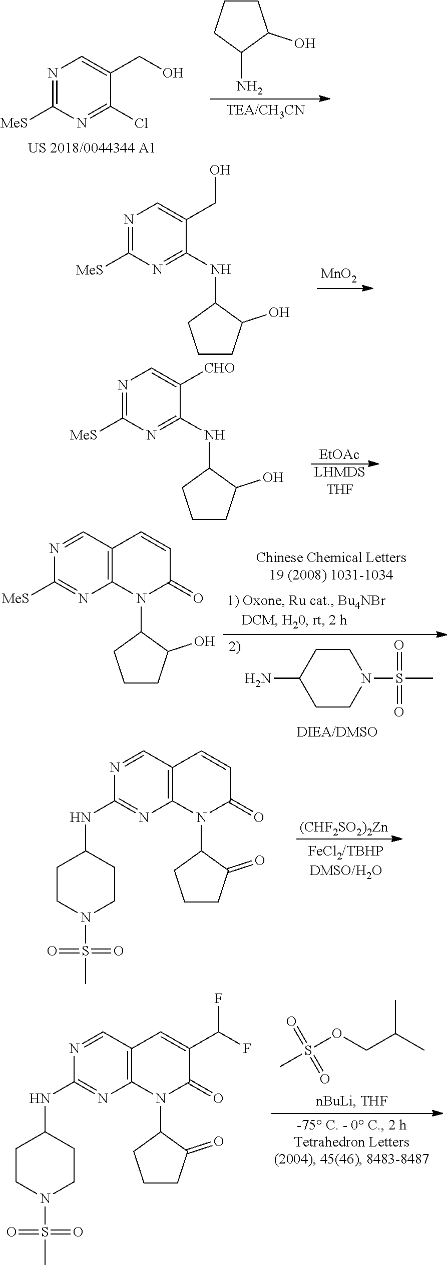

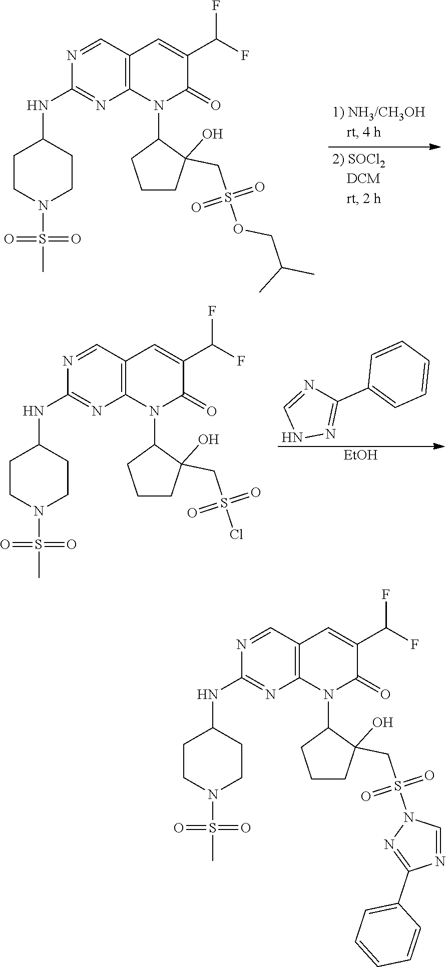

### Synthesis of Compound 110

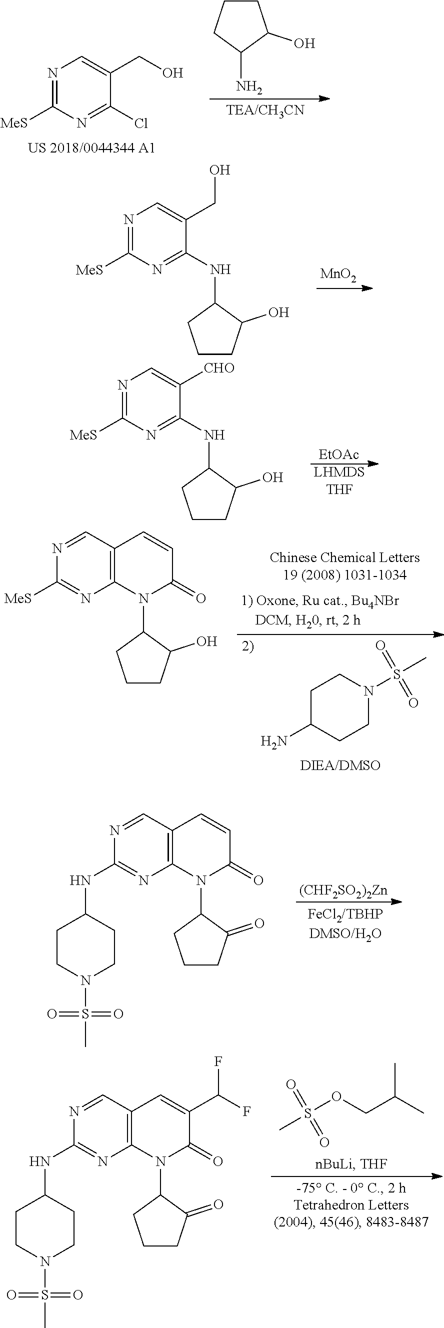

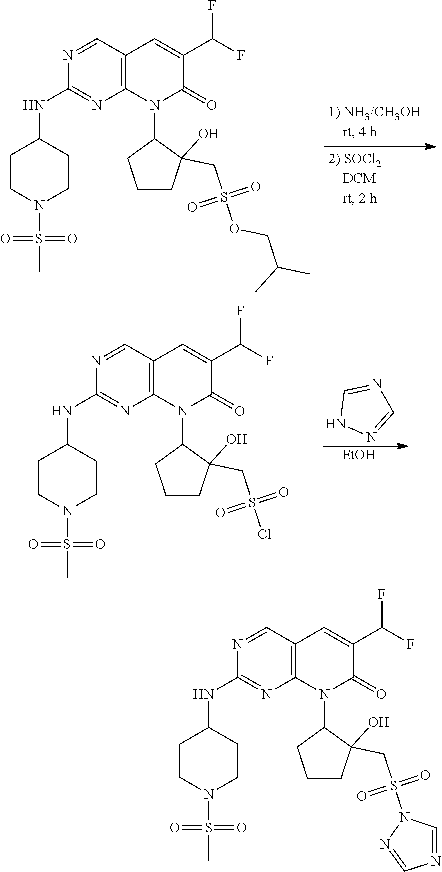

### Synthesis of Compound 111

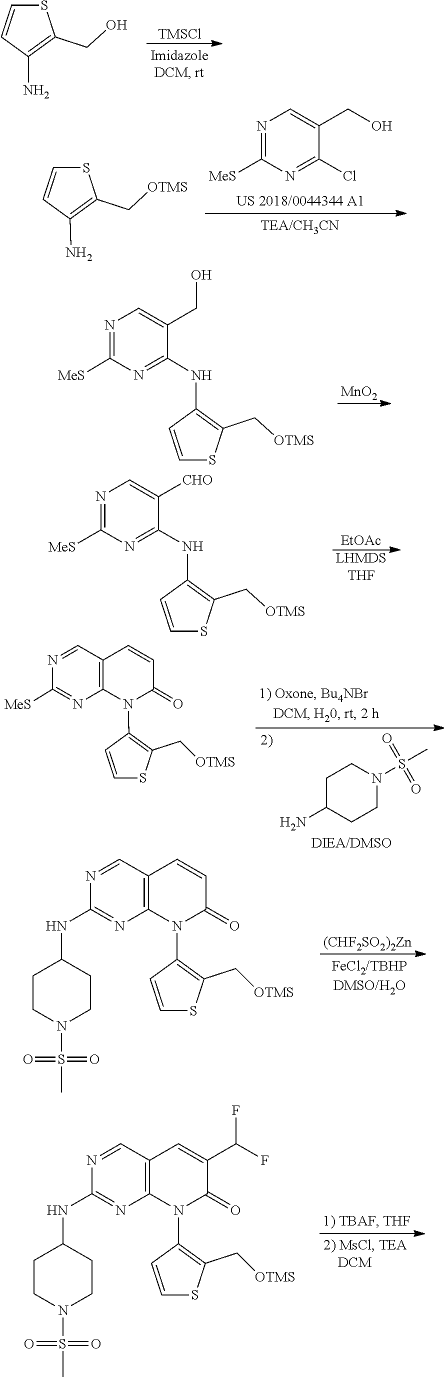

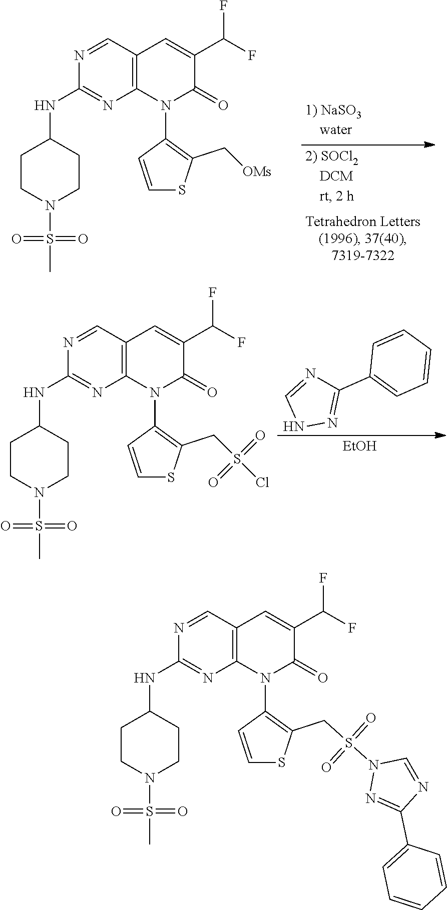

### Synthesis of Compound 112

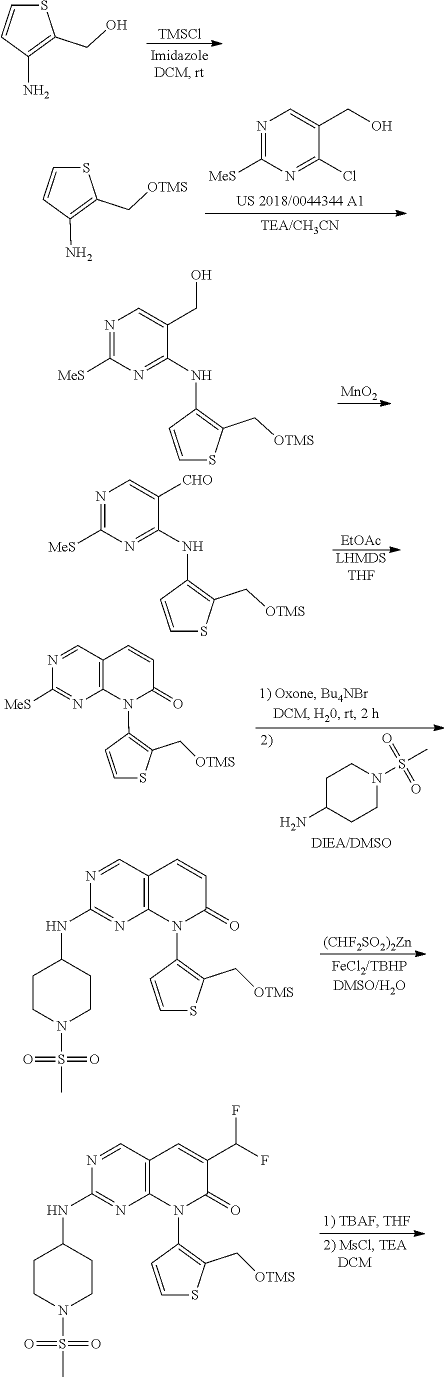

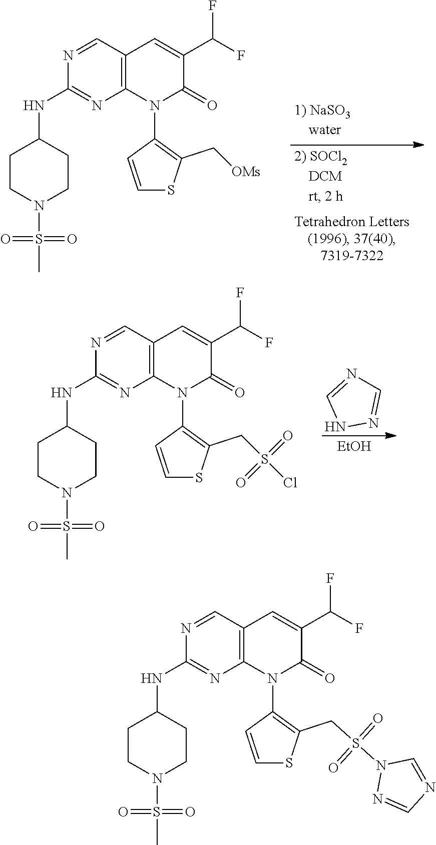

### Synthesis of Compound 113

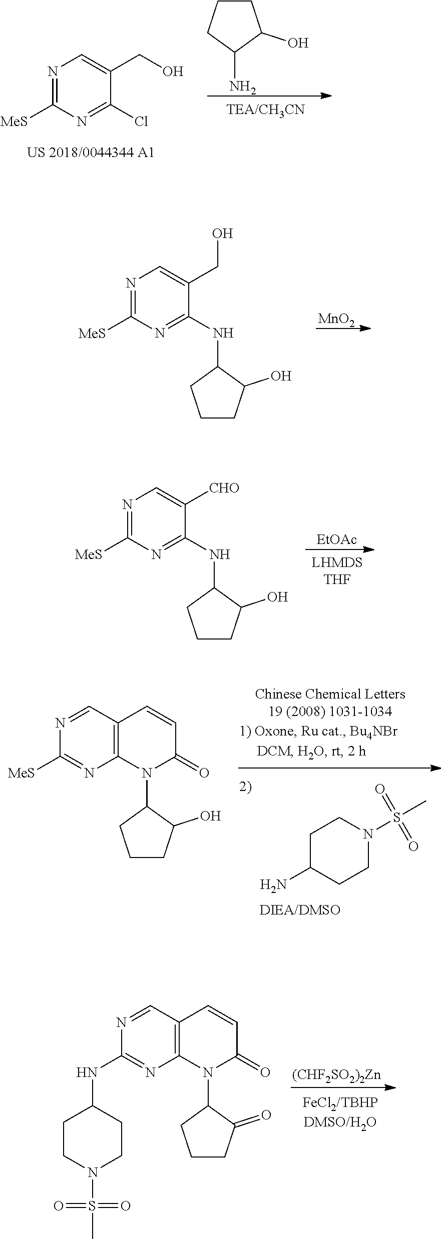

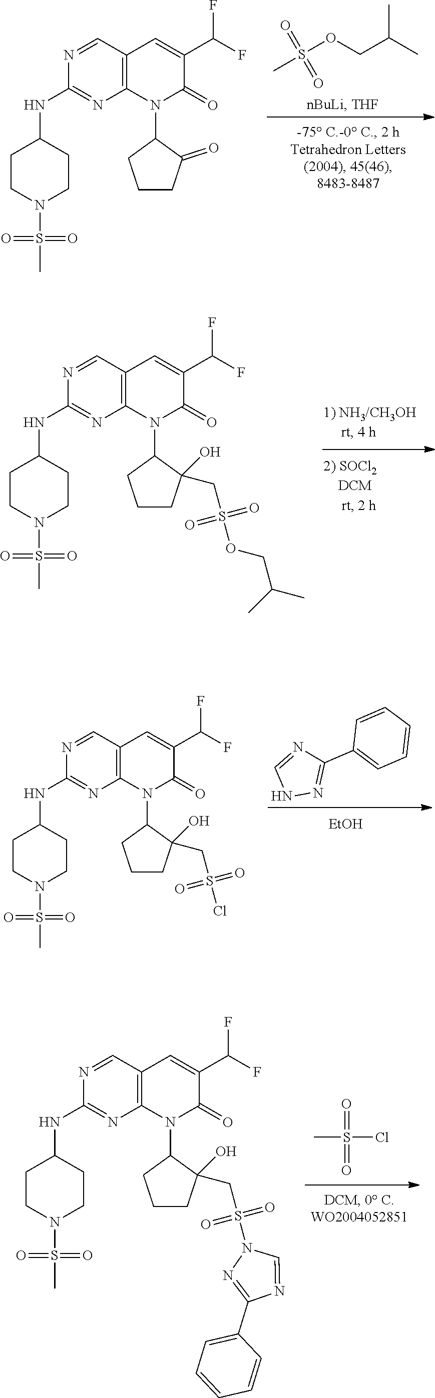

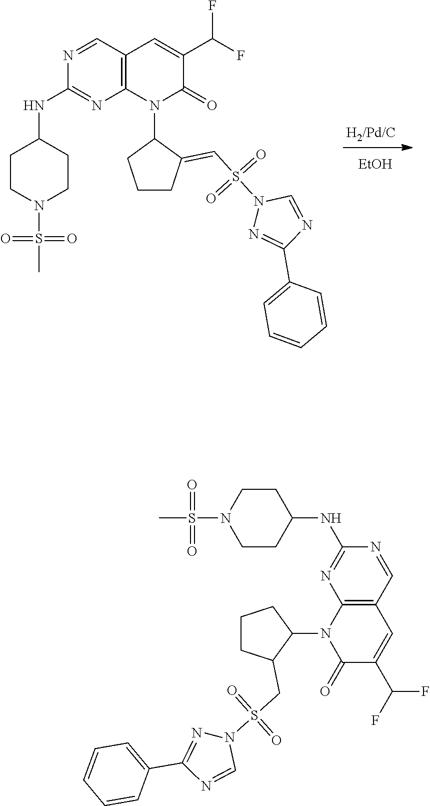 [SMILES: "EtOH" ; CS(=O)(=O)N1CCC(Nc2ncc3cc(C(F)F)c(=O)n(C4CCC/C4=C\S(=O)(=O)n4cnc(-c5ccccc5)n4)c3n2)CC1 ; "H2/Pd/C" ; CS(=O)(=O)N1CCC(Nc2ncc3cc(C(F)F)c(=O)n(C4CCCC4CS(=O)(=O)n4cnc(-c5ccccc5)n4)c3n2)CC1]

### Synthesis of Compound 114

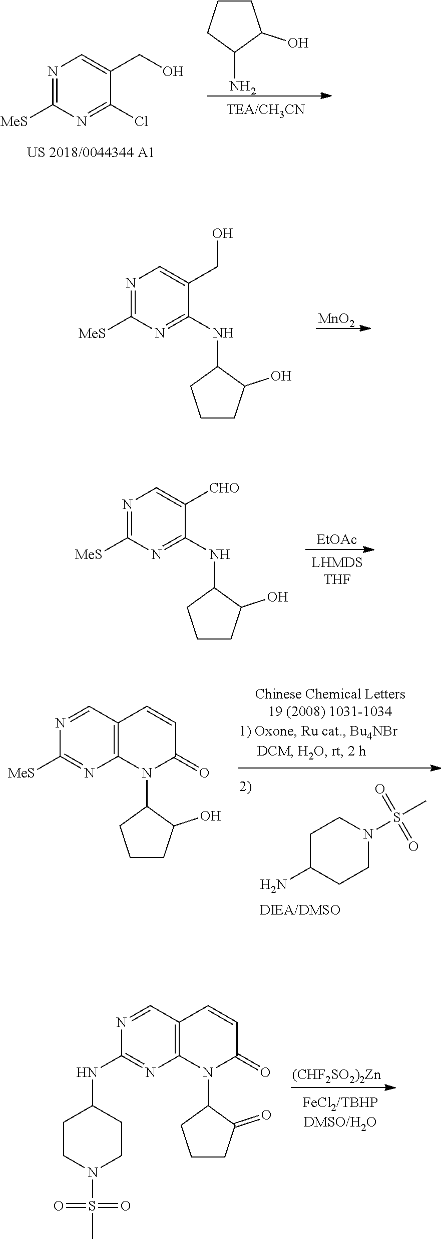

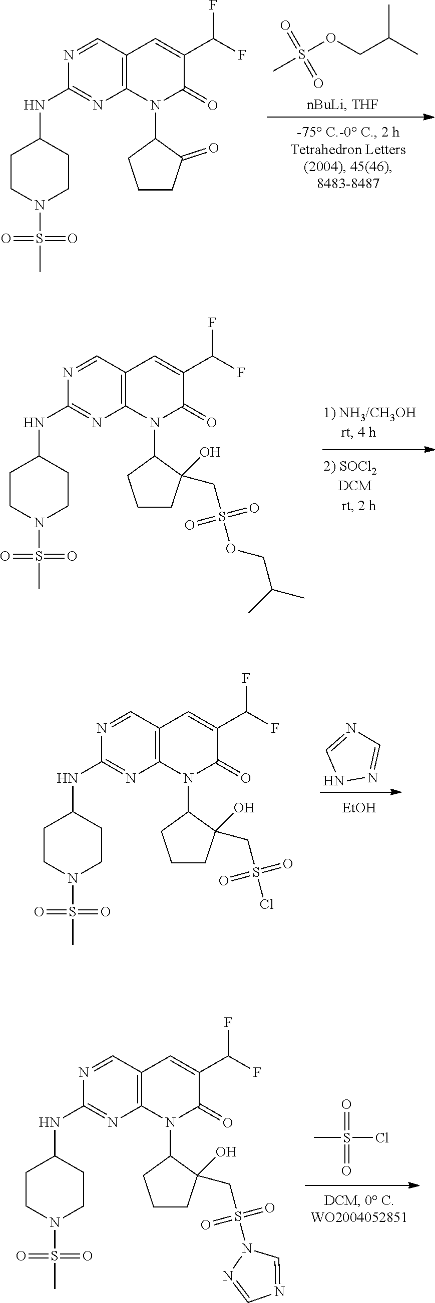

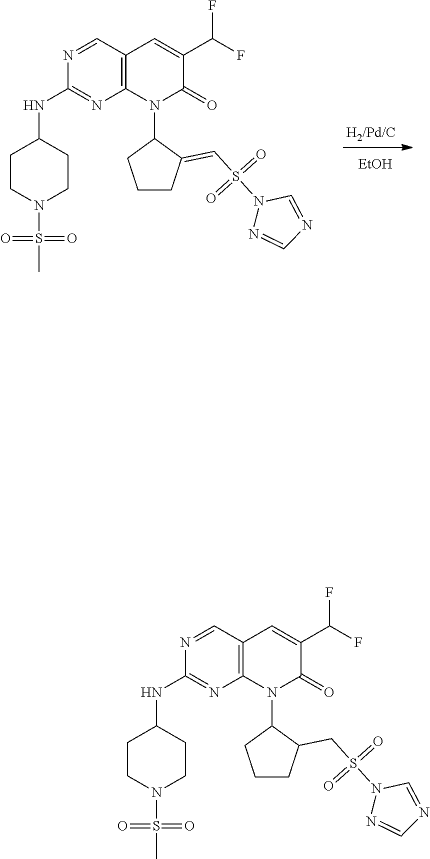 [SMILES: "EtOH" ; CS(=O)(=O)N1CCC(Nc2ncc3cc(C(F)F)c(=O)n(C4CCC/C4=C\S(=O)(=O)n4cncn4)c3n2)CC1 ; "H2/Pd/C" ; CS(=O)(=O)N1CCC(Nc2ncc3cc(C(F)F)c(=O)n(C4CCCC4CS(=O)(=O)n4cncn4)c3n2)CC1]

### Synthesis of Compound 115

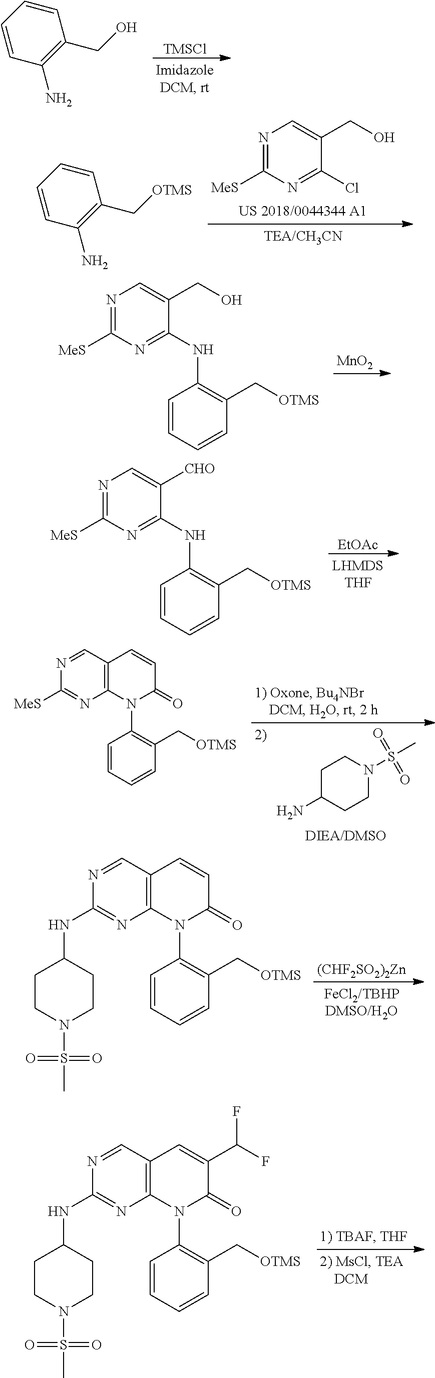

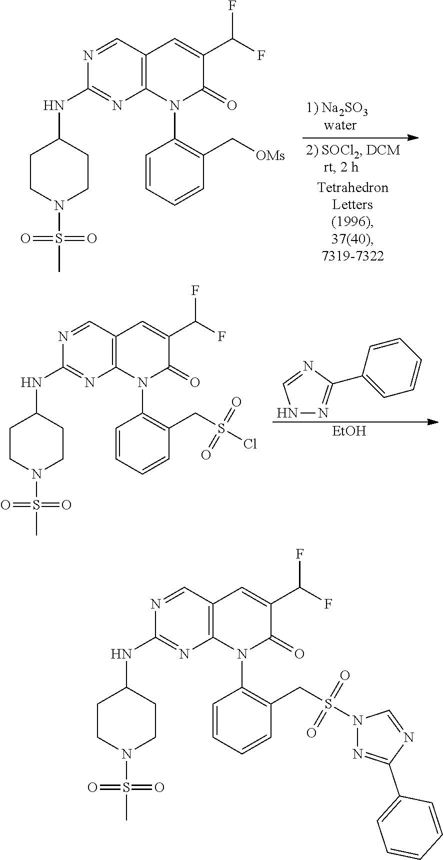 [SMILES: "EtOH" ; CS(=O)(=O)OCc1ccccc1-n1c(=O)c(C(F)F)cc2cnc(NC3CCN(S(C)(=O)=O)CC3)nc21 ; CS(=O)(=O)N1CCC(Nc2ncc3cc(C(F)F)c(=O)n(-c4ccccc4CS(=O)(=O)Cl)c3n2)CC1 ; c1ccc(-c2nc[nH]n2)cc1 ; CS(=O)(=O)N1CCC(Nc2ncc3cc(C(F)F)c(=O)n(-c4ccccc4CS(=O)(=O)n4cnc(-c5ccccc5)n4)c3n2)CC1]

### Synthesis of Compound 116

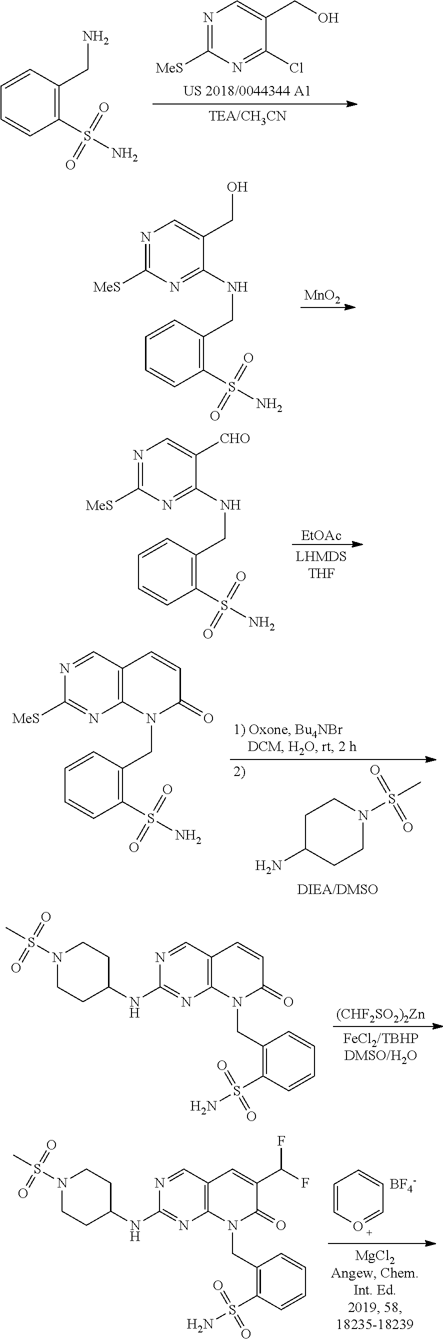

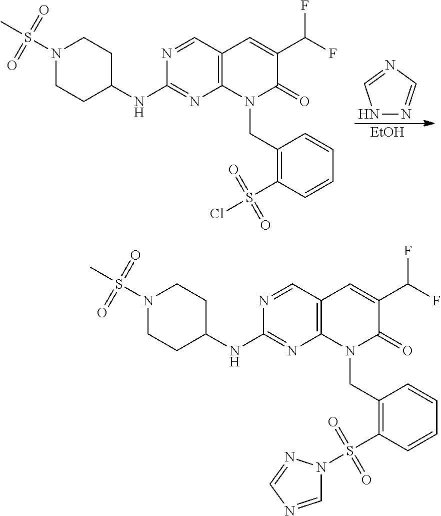

### Synthesis of Compound 117

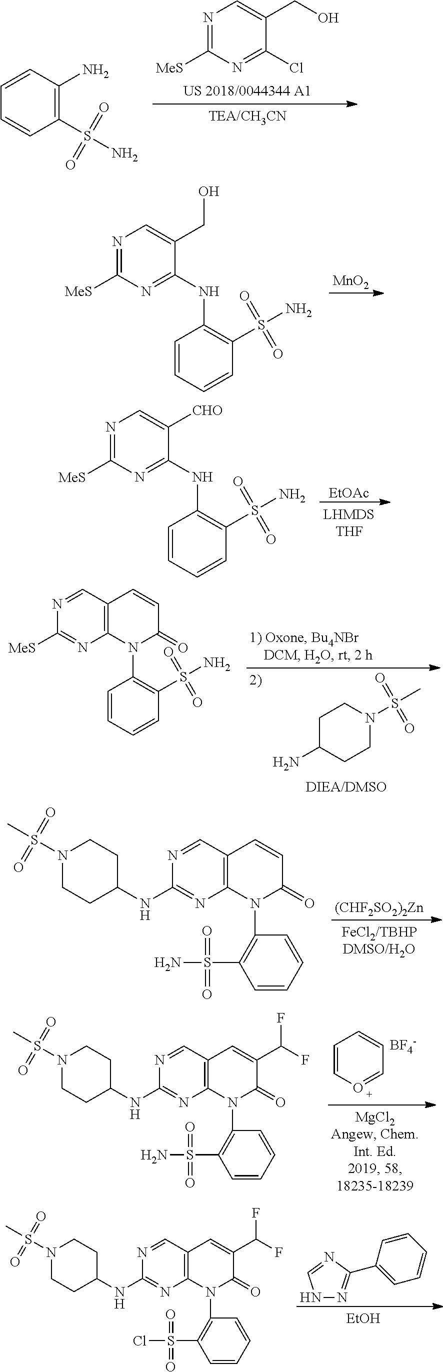

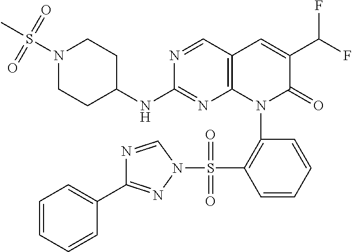 [SMILES: CS(=O)(=O)N1CCC(Nc2ncc3cc(C(F)F)c(=O)n(-c4ccccc4S(=O)(=O)n4cnc(-c5ccccc5)n4)c3n2)CC1]

### Synthesis of Compound 118

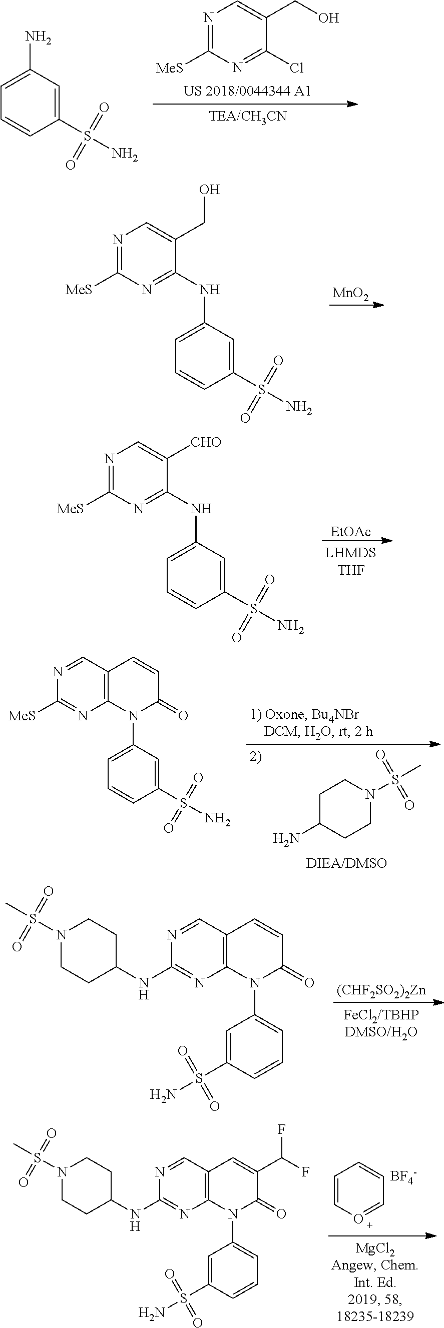

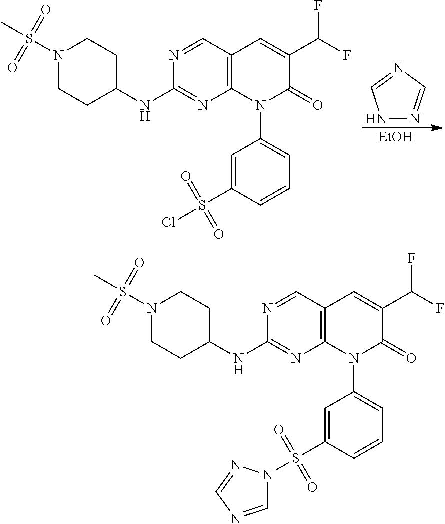

### Synthesis of Compound 119

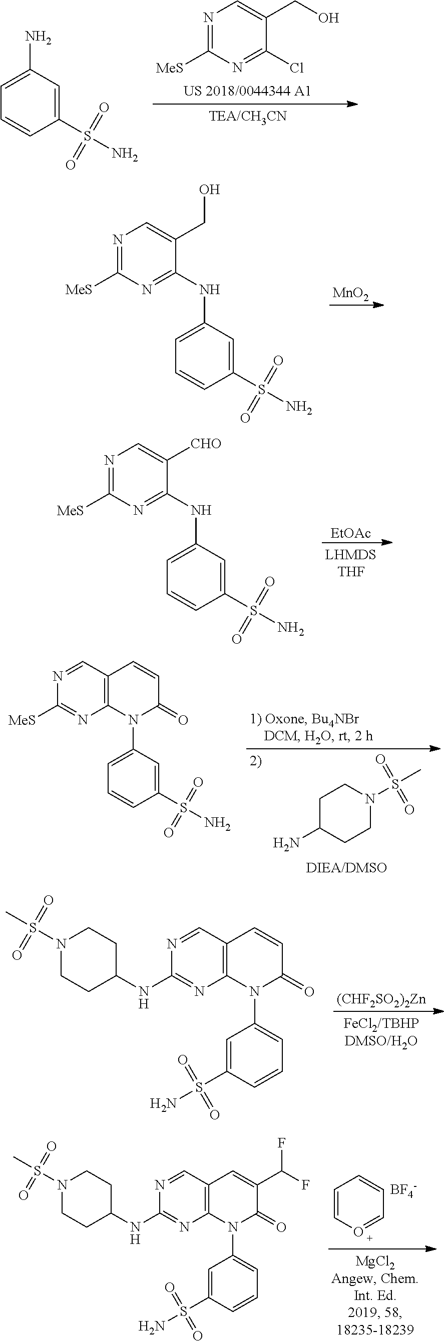

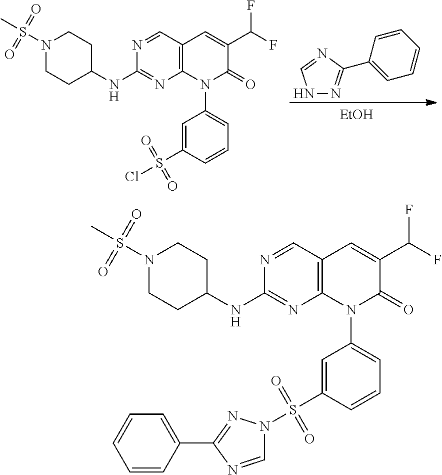

### Synthesis of Compound 120

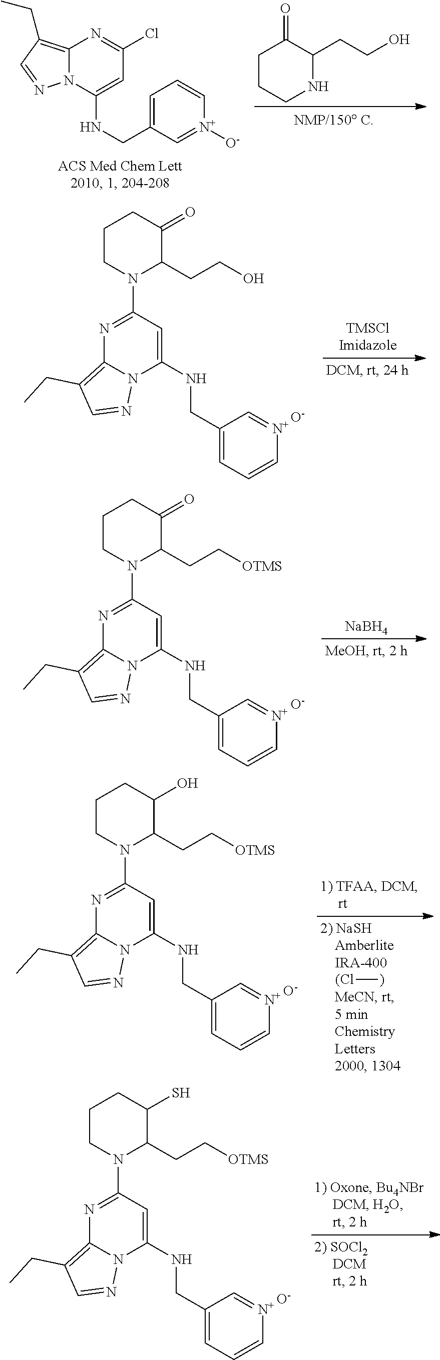

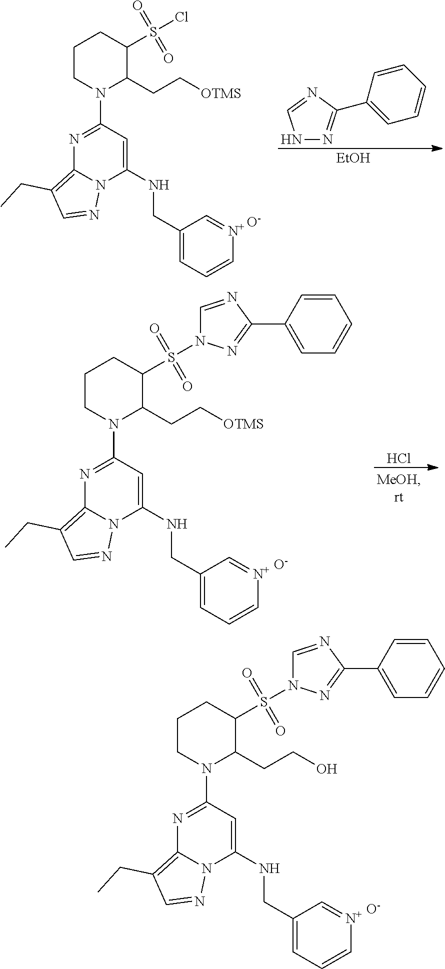

### Synthesis of Compound 121

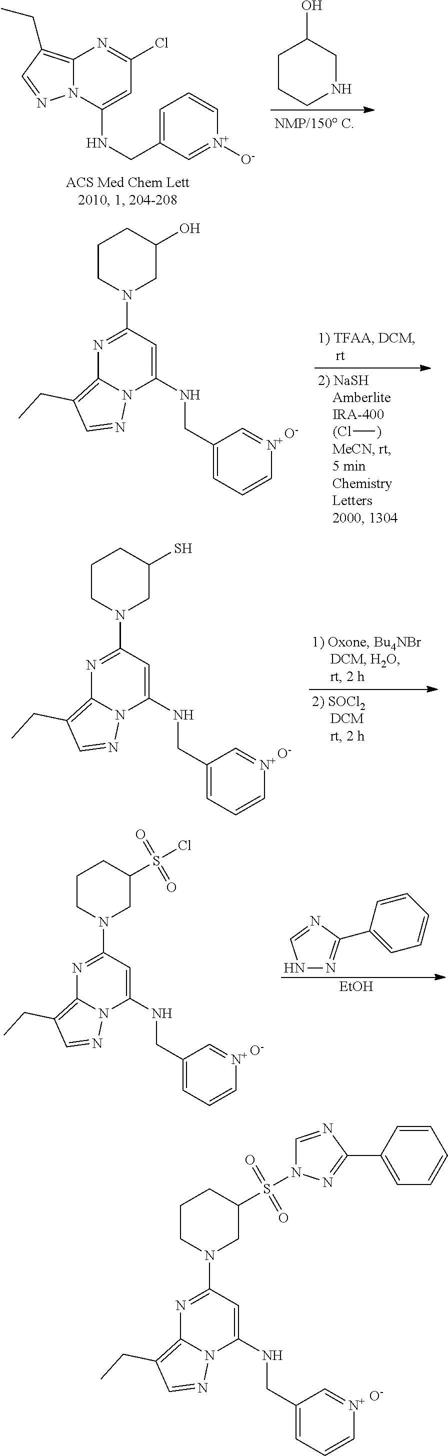

### Synthesis of Compound 122

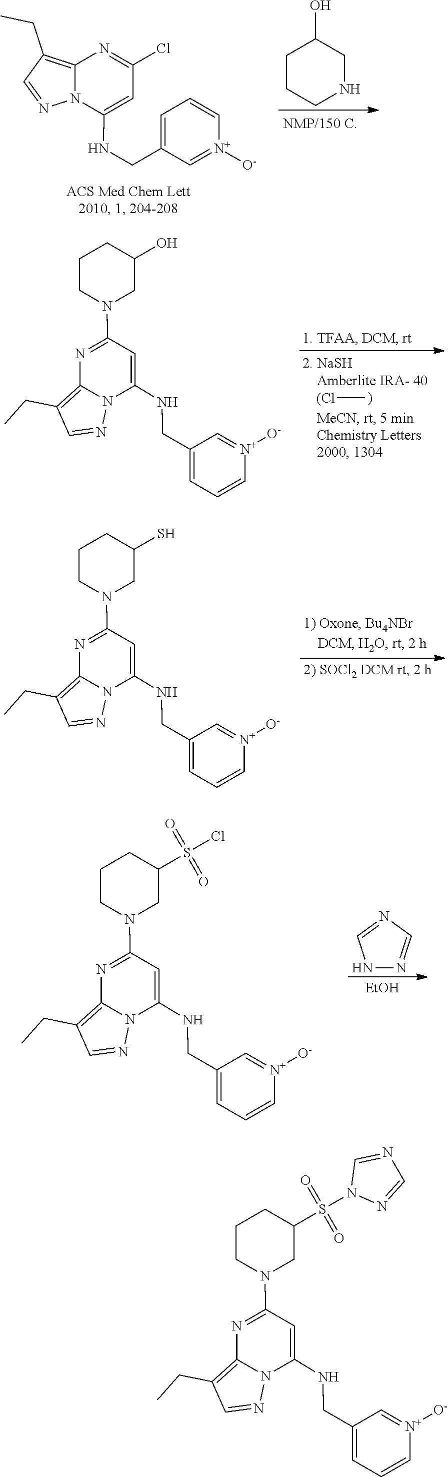

### Synthesis of Compound 123

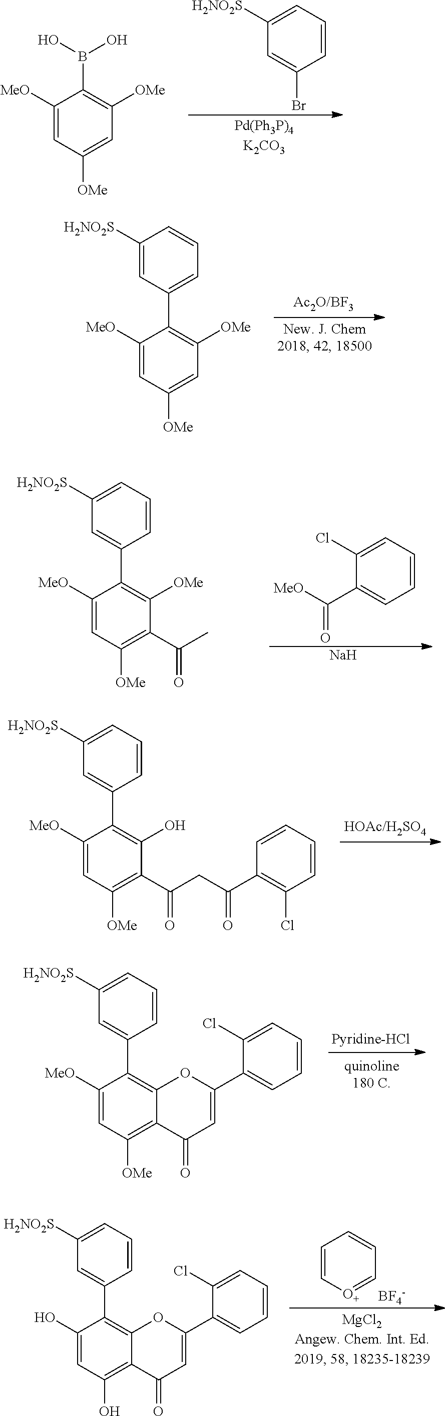

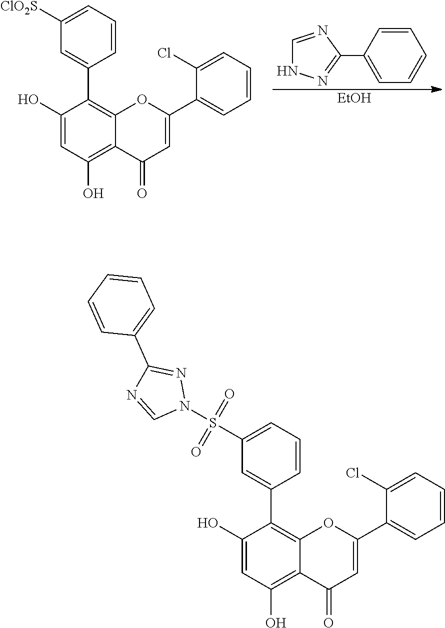

### Synthesis of Compound 124

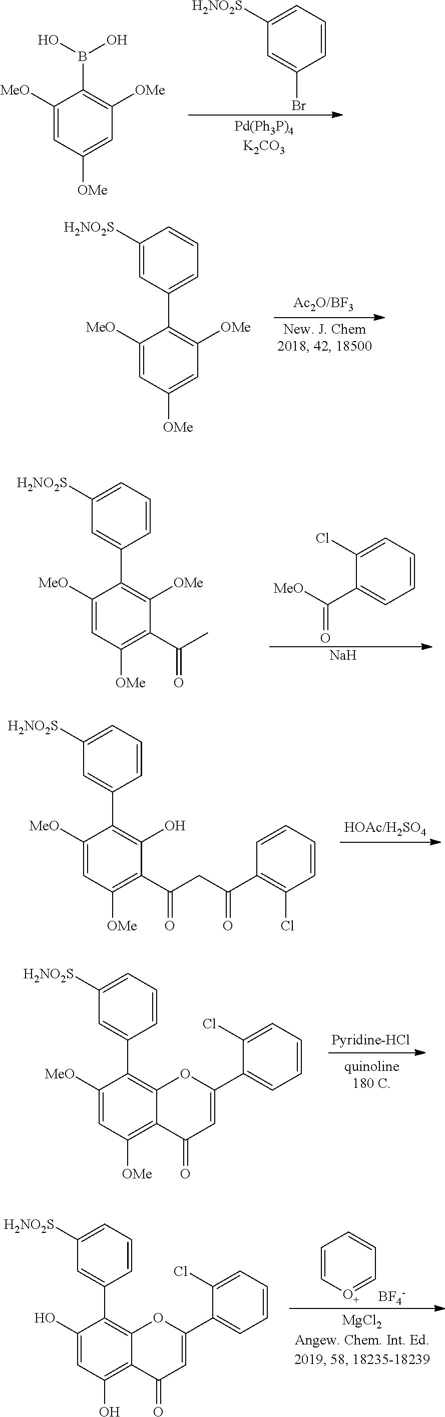

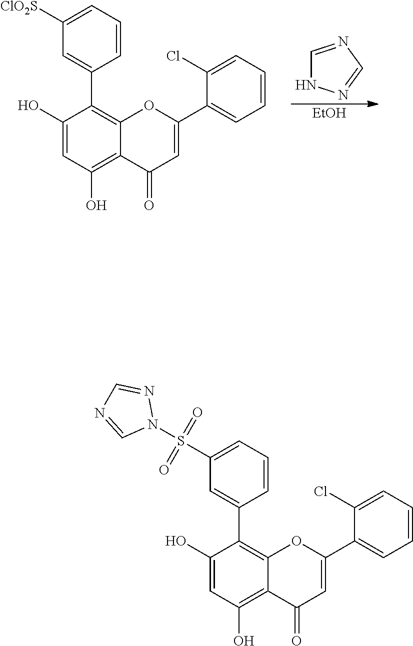 [SMILES: "EtOH" ; O=c1cc(-c2ccccc2Cl)oc2c(-c3cccc(S(=O)(=O)Cl)c3)c(O)cc(O)c12 ; c1nc[nH]n1 ; O=c1cc(-c2ccccc2Cl)oc2c(-c3cccc(S(=O)(=O)n4cncn4)c3)c(O)cc(O)c12]

### Synthesis of Compound 125

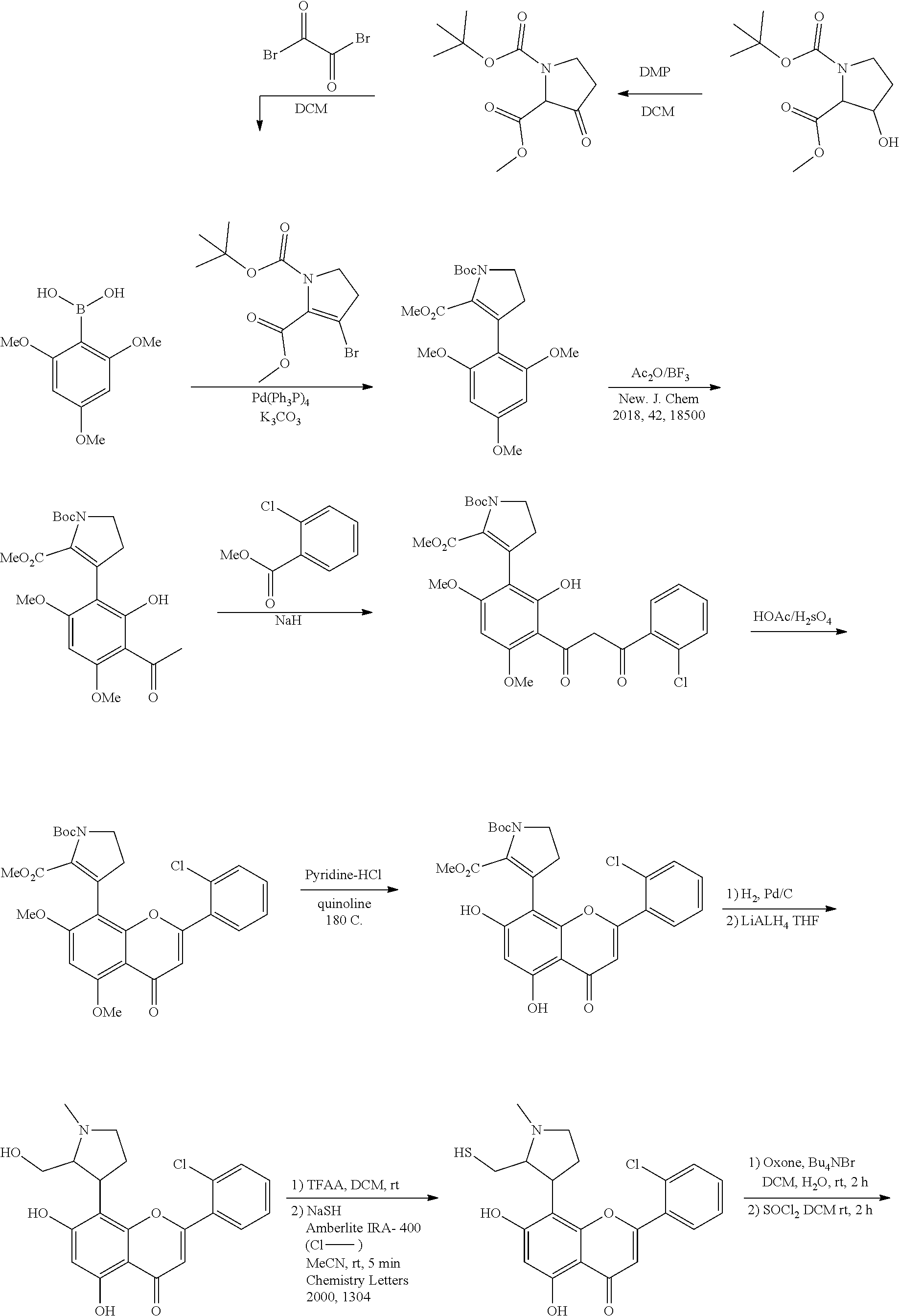

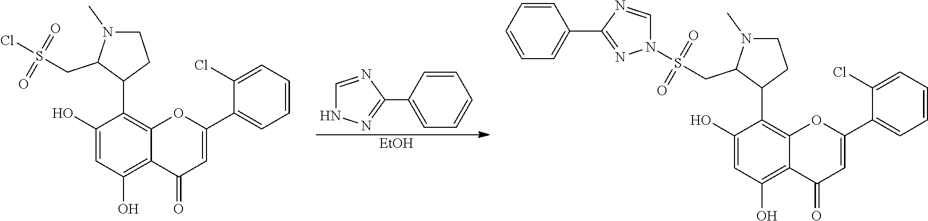 [SMILES: "EtOH" ; CN1CCC(c2c(O)cc(O)c3c(=O)cc(-c4ccccc4Cl)oc23)C1CS(=O)(=O)Cl ; c1ccc(-c2nc[nH]n2)cc1 ; CN1CCC(c2c(O)cc(O)c3c(=O)cc(-c4ccccc4Cl)oc23)C1CS(=O)(=O)n1cnc(-c2ccccc2)n1]

### Synthesis of Compound 126

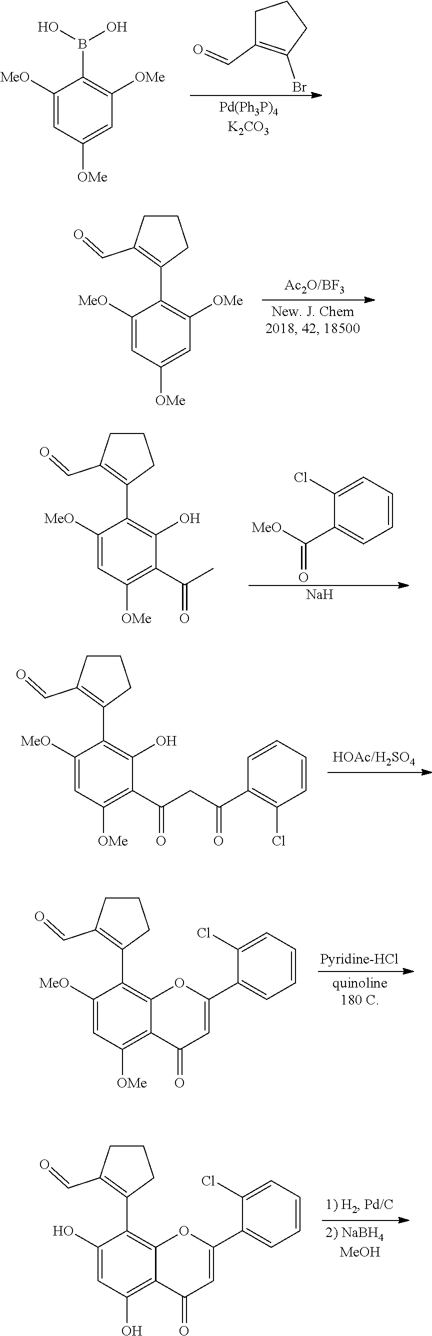

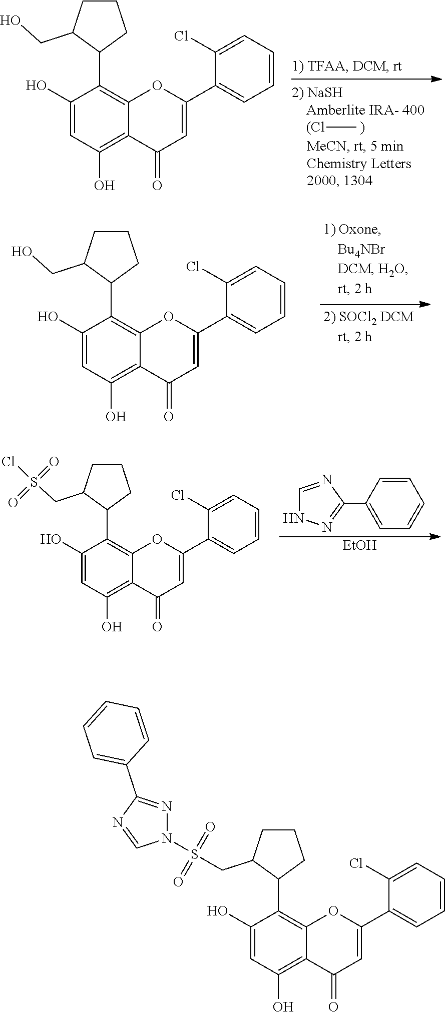

### Synthesis of Compound 127

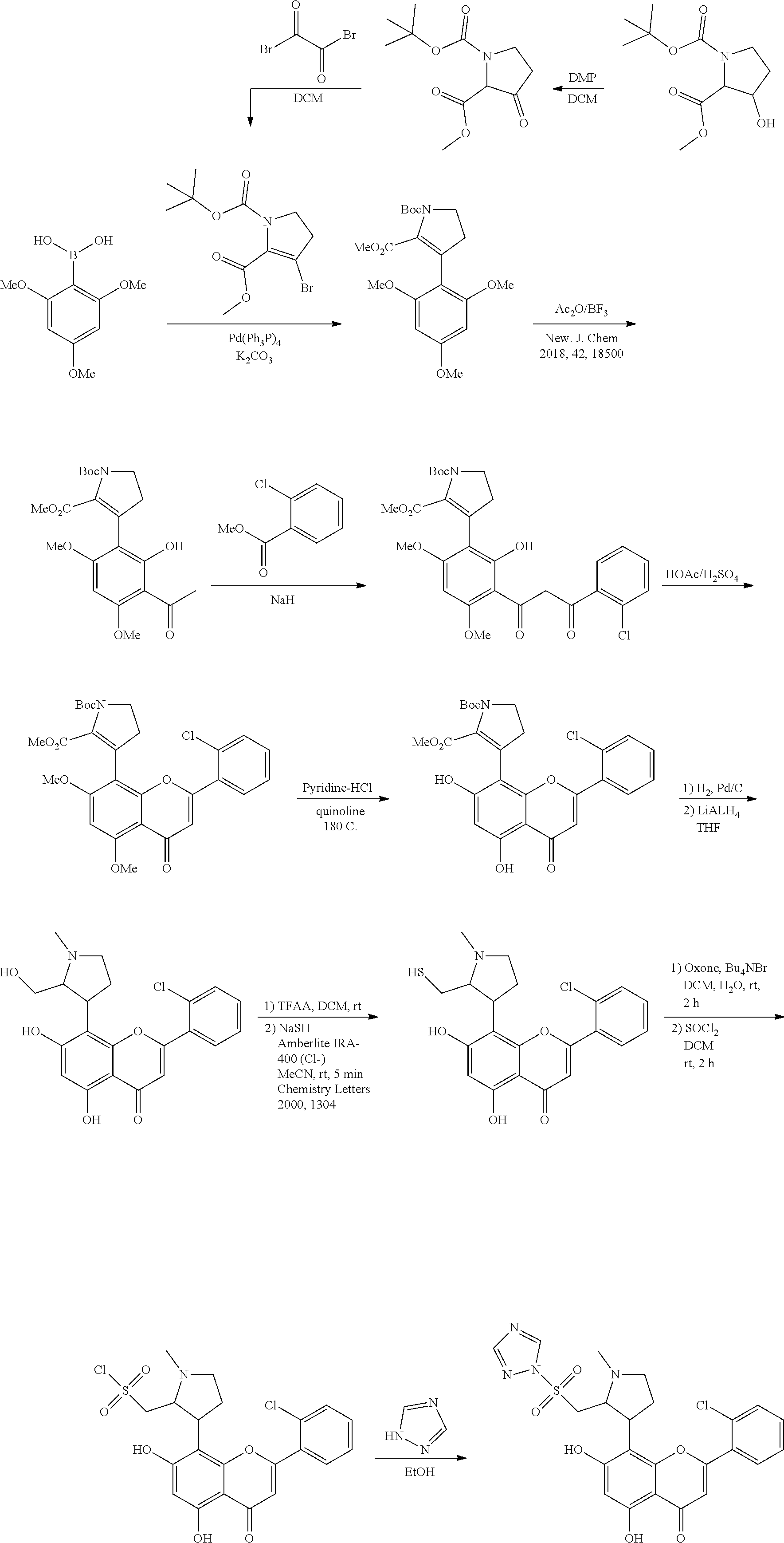

### Synthesis of Compound 128

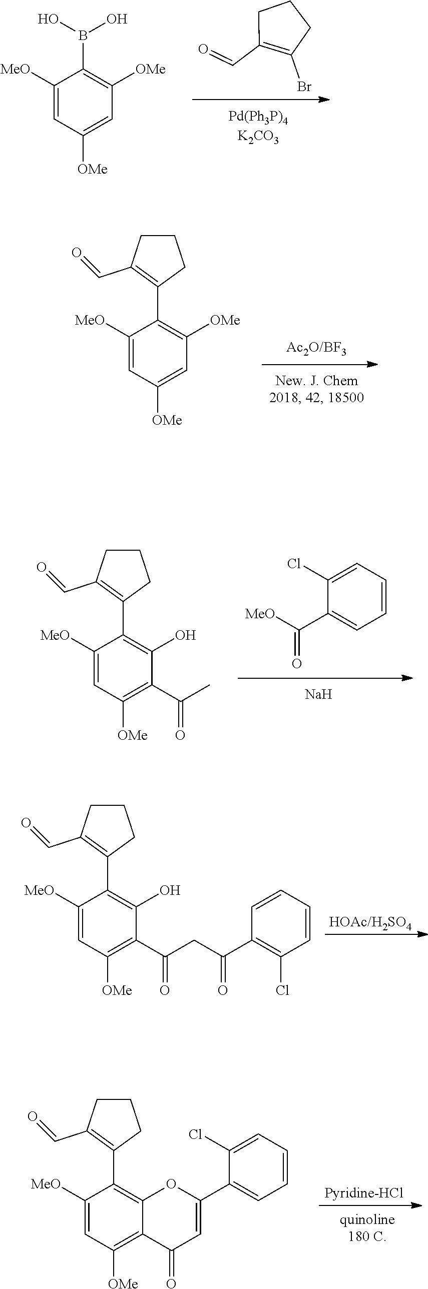

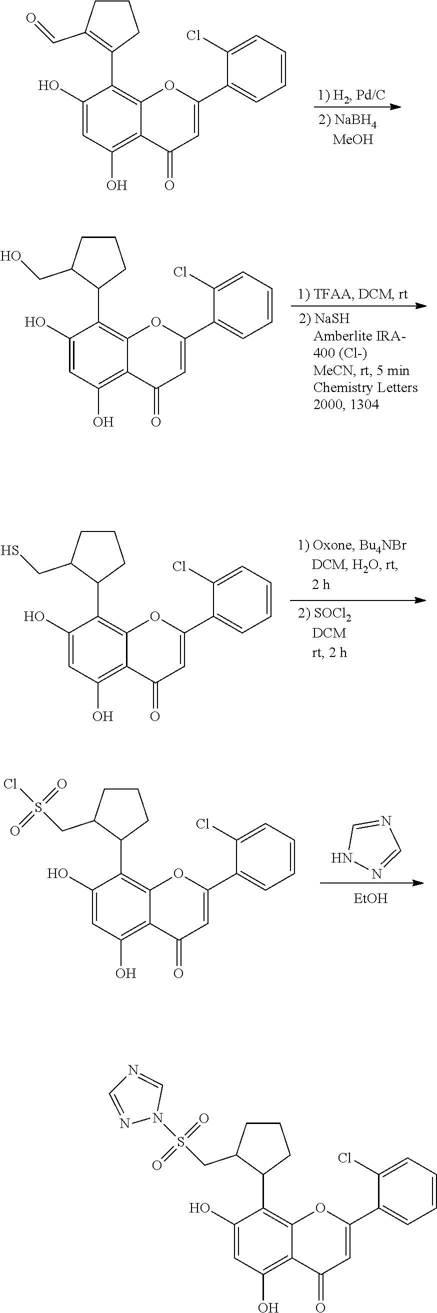

### Synthesis of Compound 129

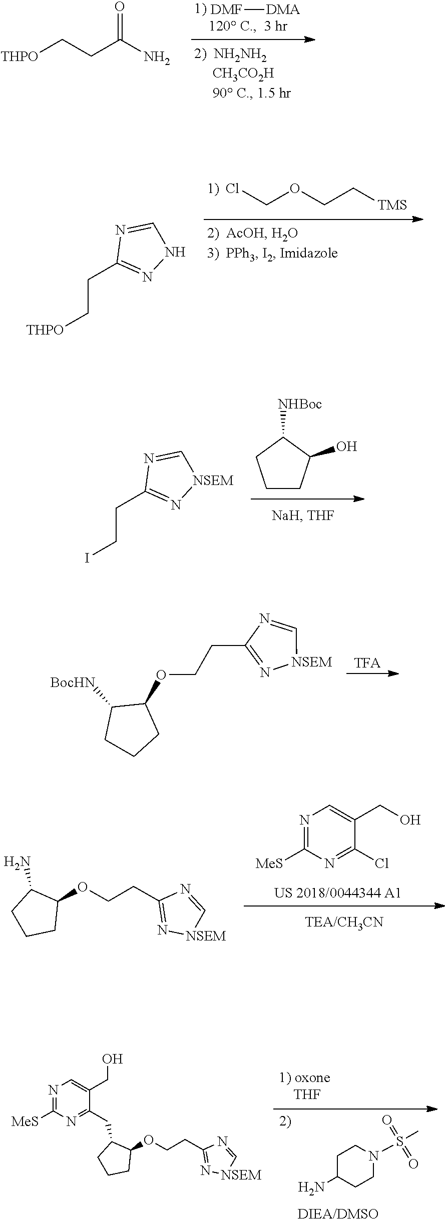

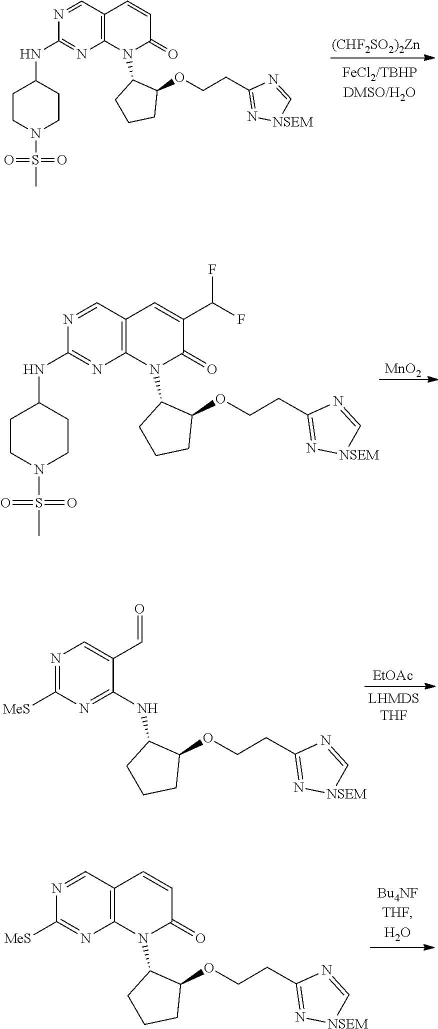

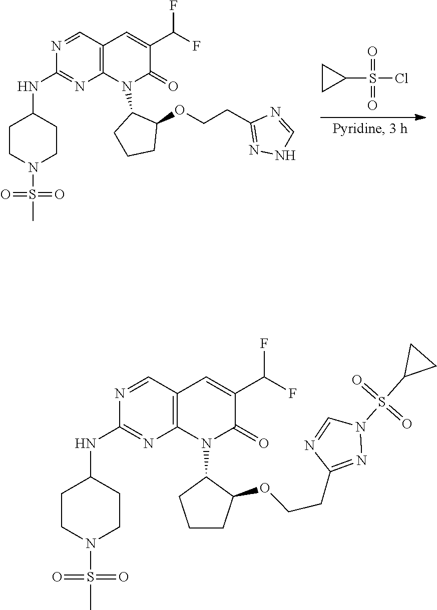

### Synthesis of Compound 130

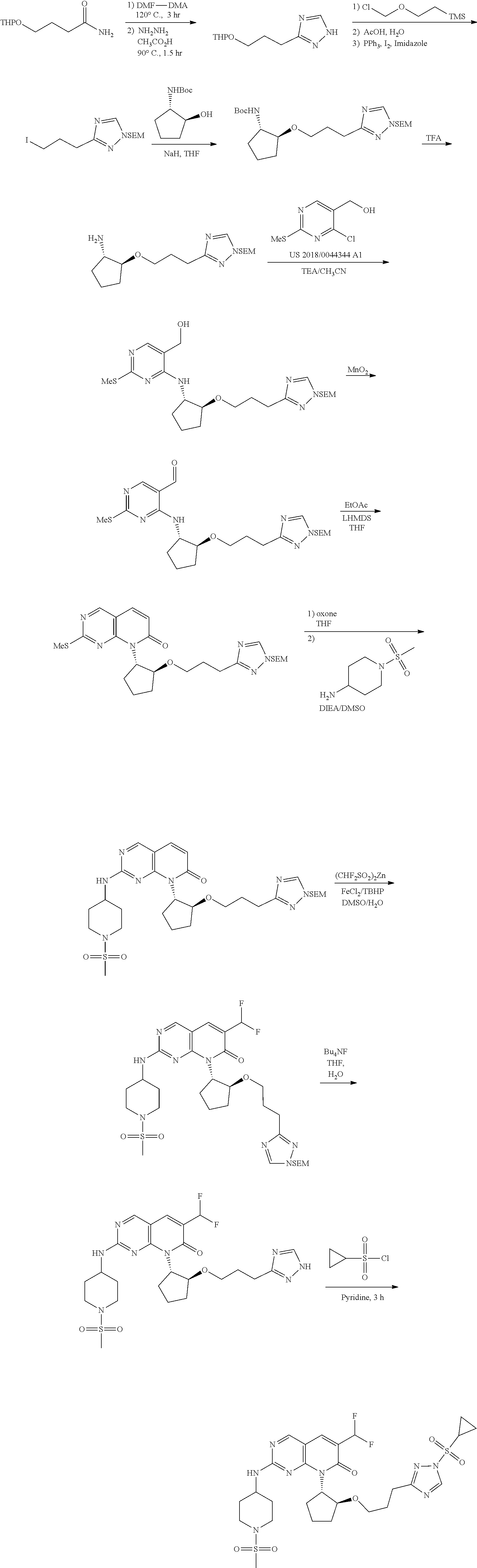

### Synthesis of Compound 131

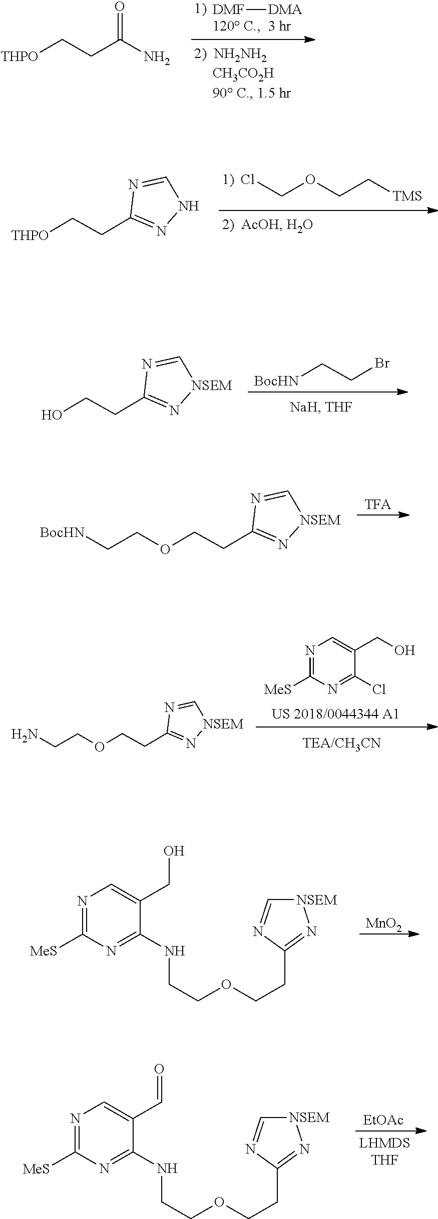

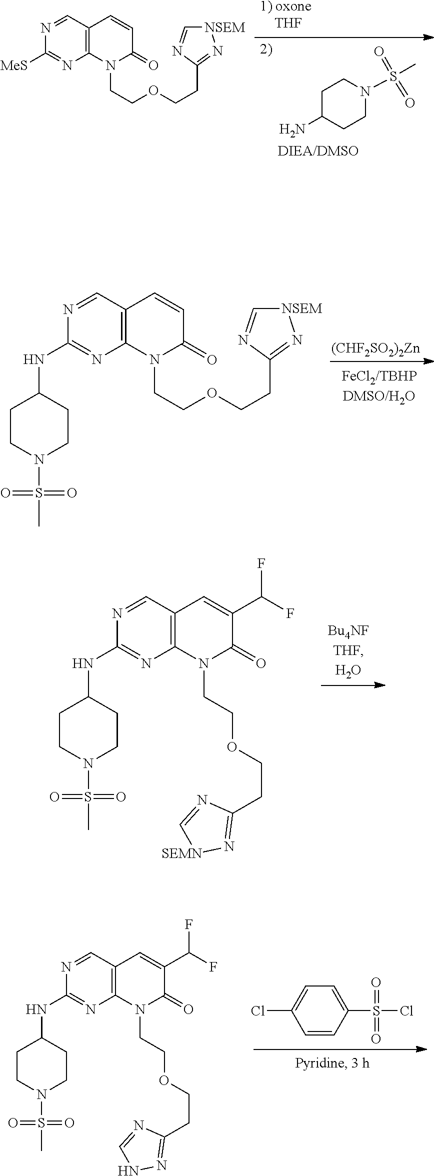

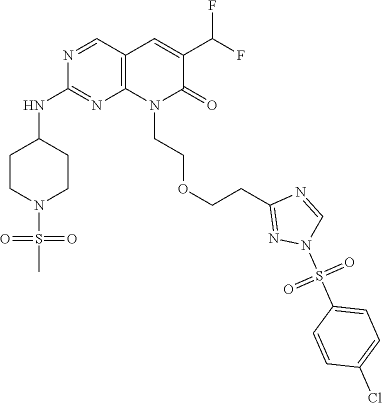 [SMILES: CS(=O)(=O)N1CCC(Nc2ncc3cc(C(F)F)c(=O)n(CCOCCc4ncn(S(=O)(=O)c5ccc(Cl)cc5)n4)c3n2)CC1]

### Synthesis of Compound 132

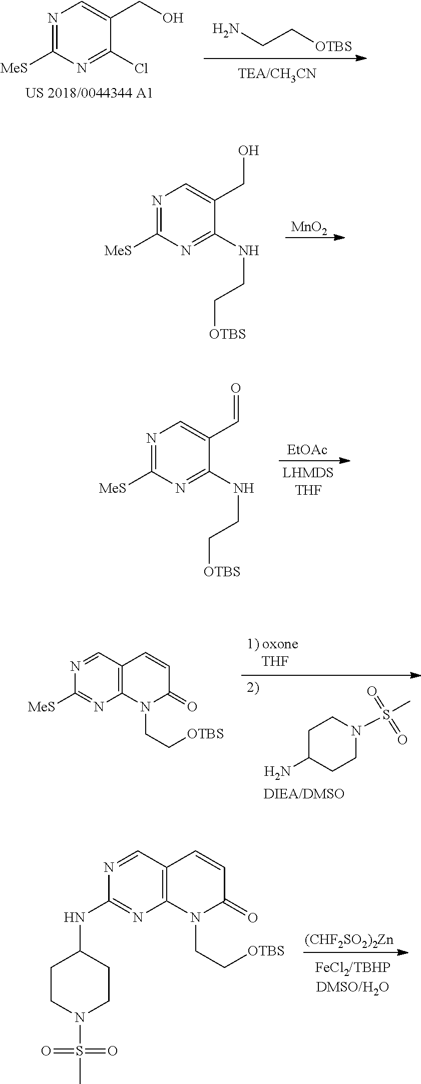

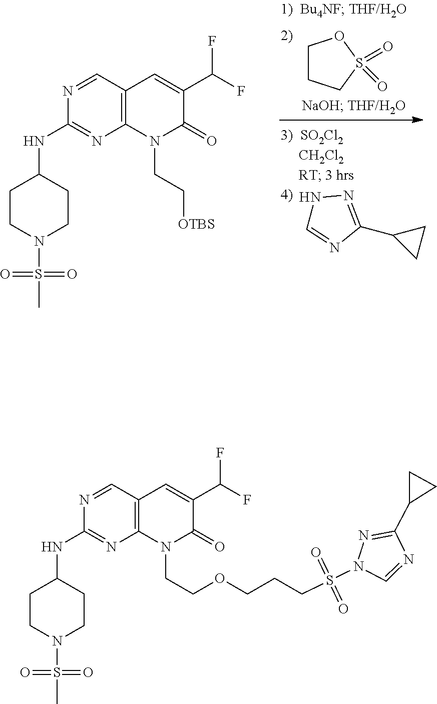 [SMILES: CC(C)(C)[Si](C)(C)OCCn1c(=O)c(C(F)F)cc2cnc(NC3CCN(S(C)(=O)=O)CC3)nc21 ; c1nc(C2CC2)n[nH]1 ; O=S1(=O)CCCO1 ; CS(=O)(=O)N1CCC(Nc2ncc3cc(C(F)F)c(=O)n(CCOCCCS(=O)(=O)n4cnc(C5CC5)n4)c3n2)CC1]

### Synthesis of Compound 133

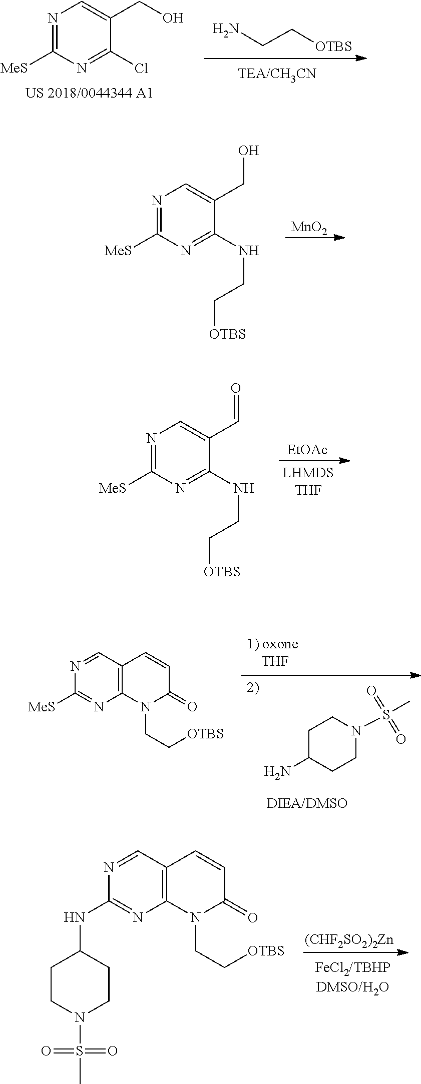

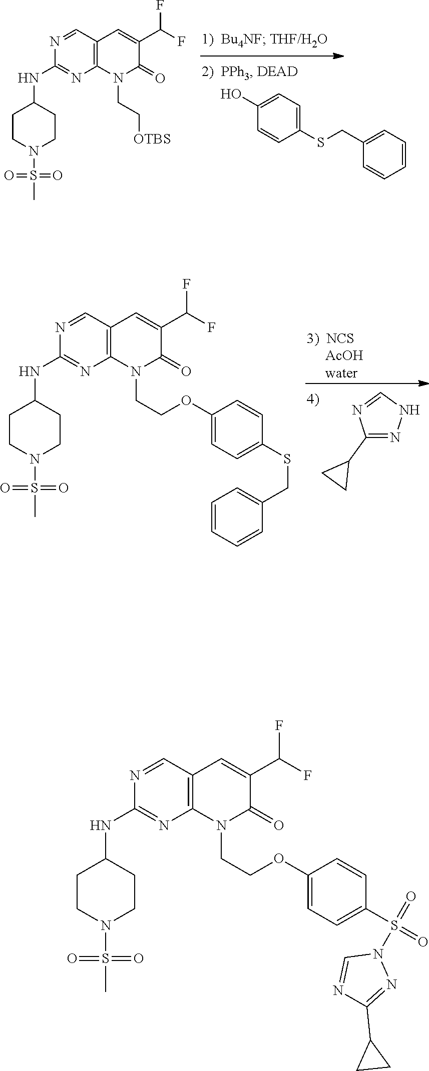

### Synthesis of Compound 134

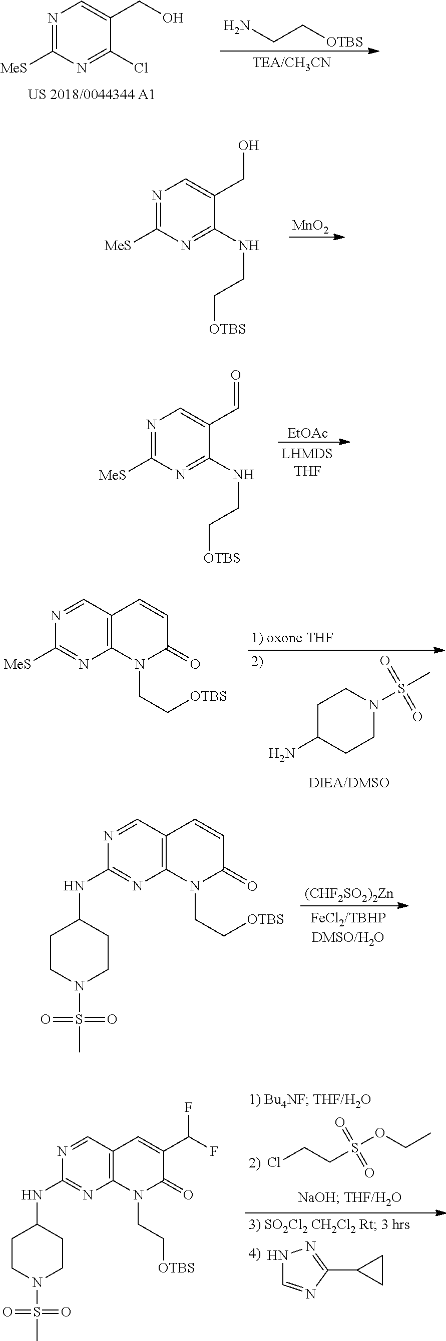

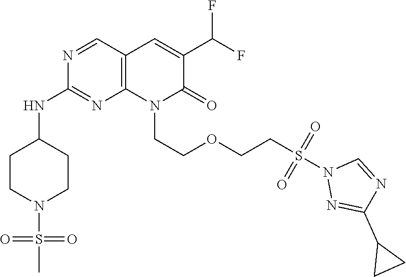 [SMILES: CS(=O)(=O)N1CCC(Nc2ncc3cc(C(F)F)c(=O)n(CCOCCS(=O)(=O)n4cnc(C5CC5)n4)c3n2)CC1]

### Synthesis of Compound 135

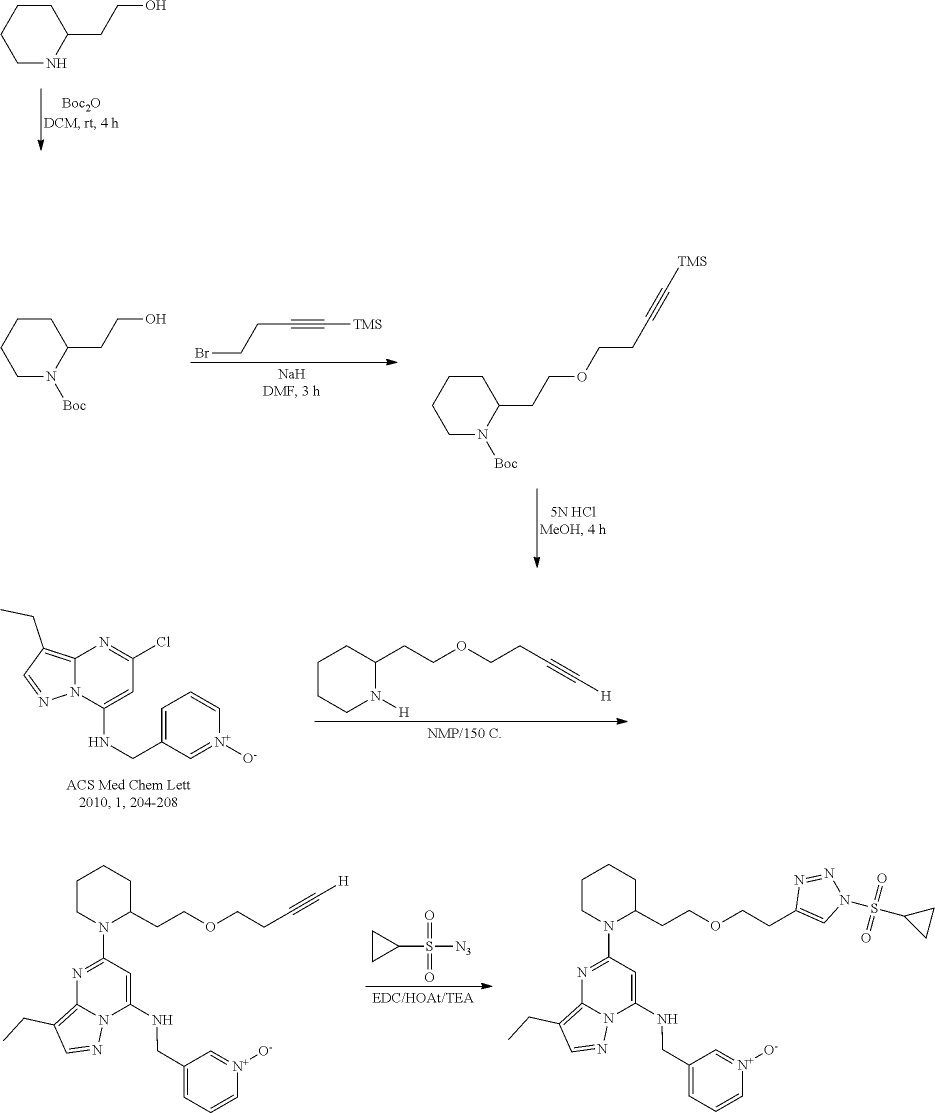

### Synthesis of Compound 136

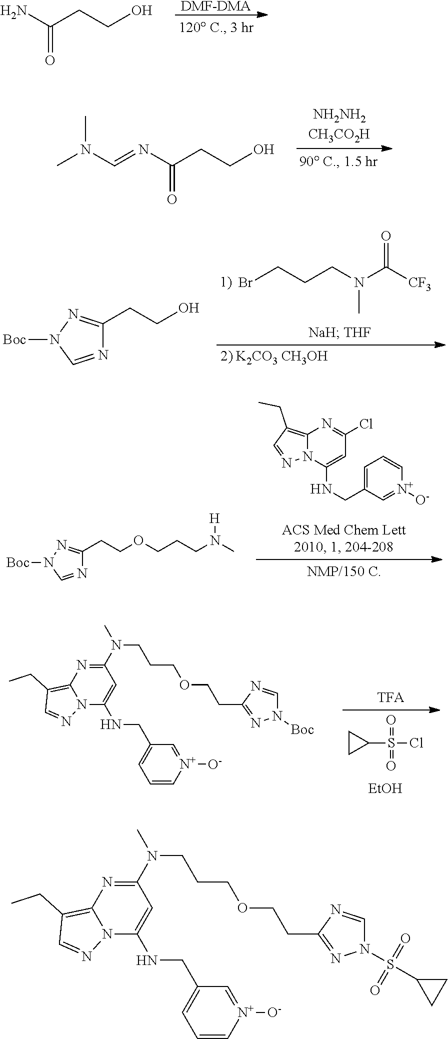

### Synthesis of Compound 137

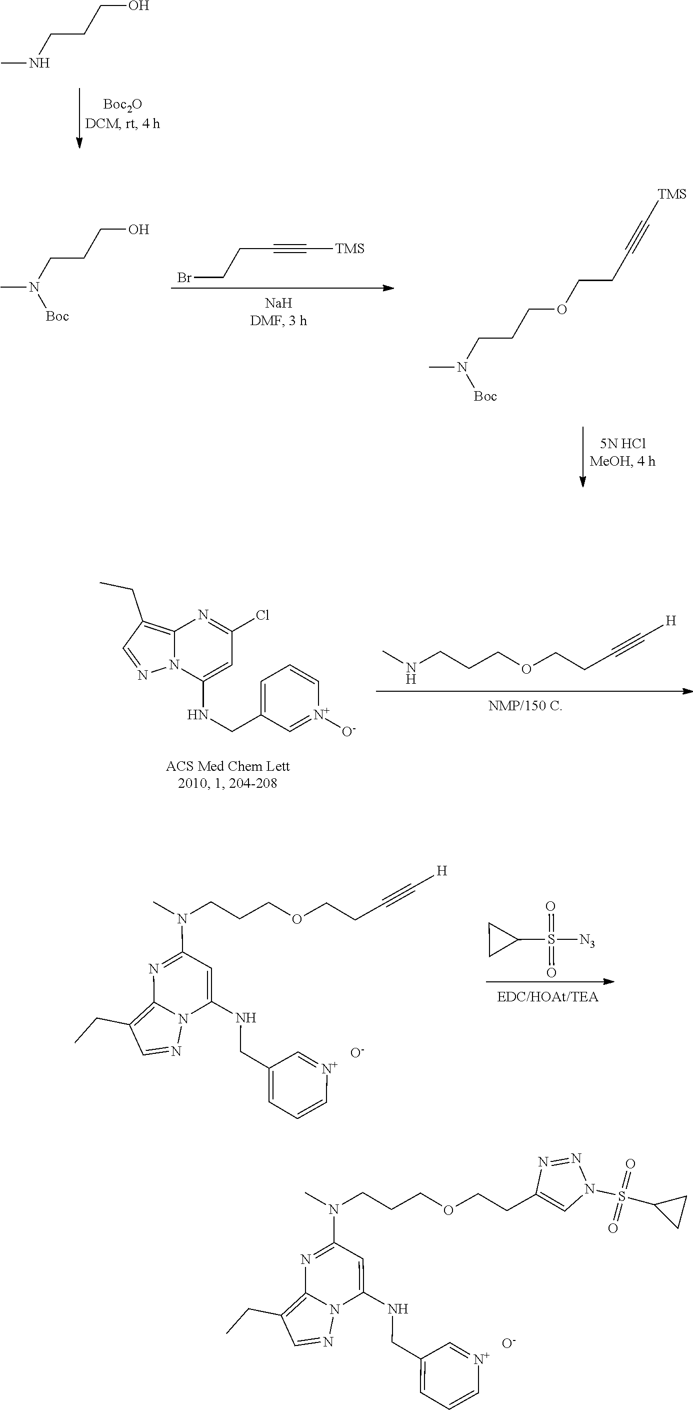

### Synthesis of Compound 138

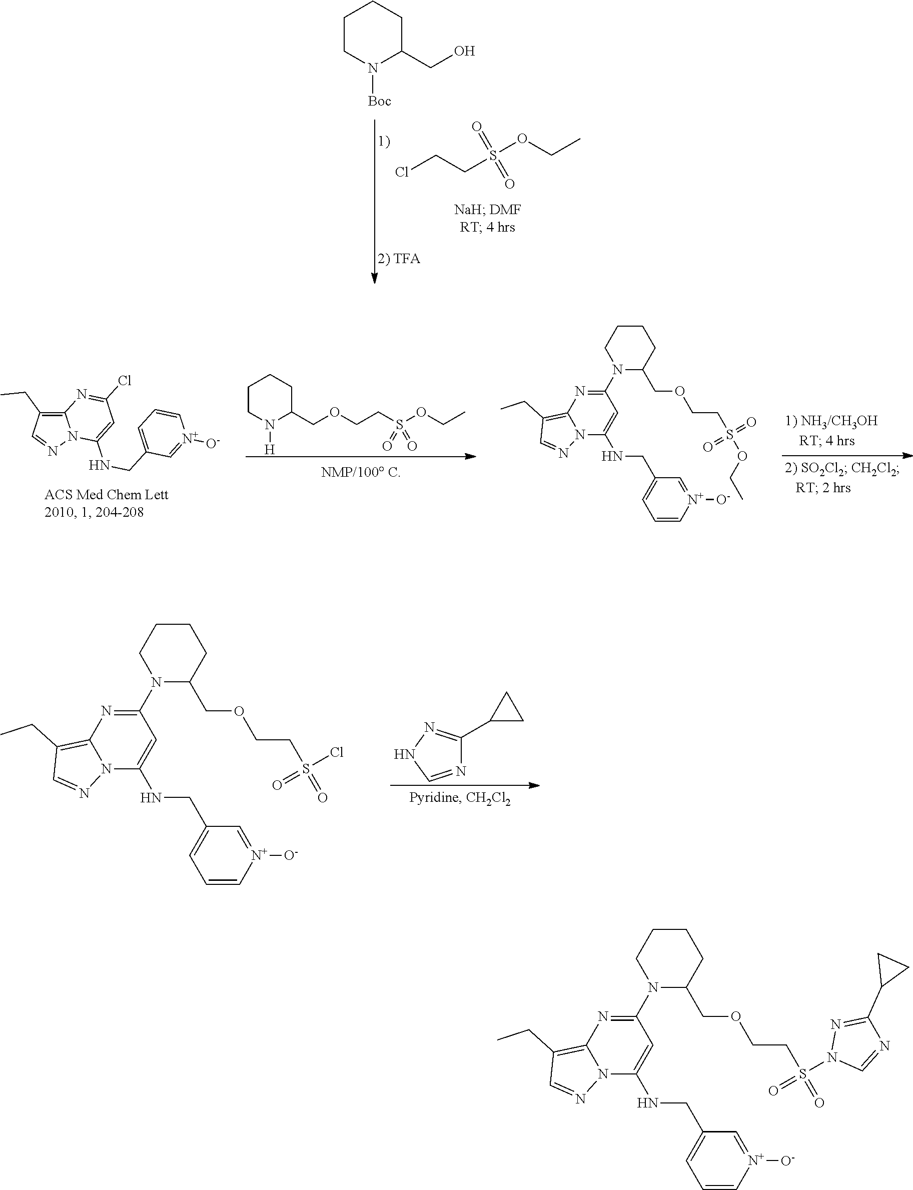

### Synthesis of Compound 139

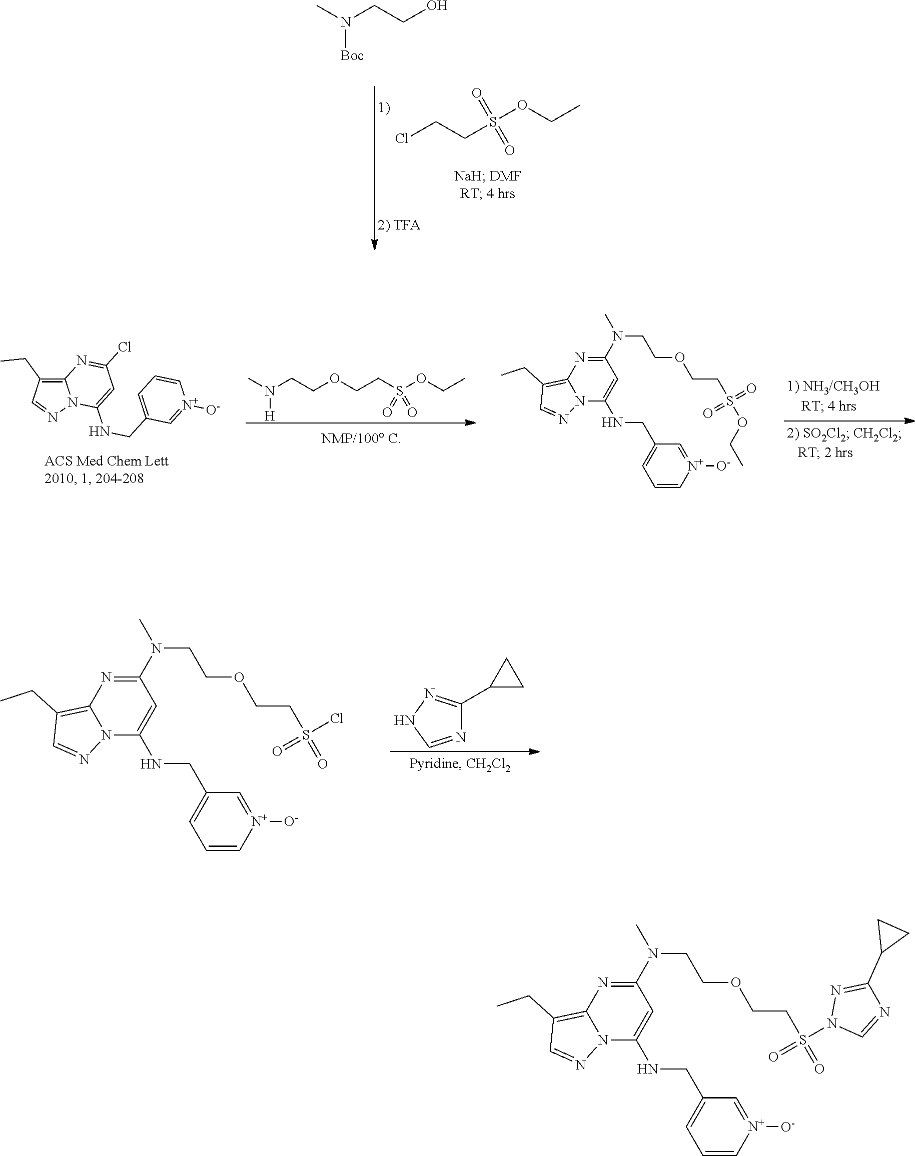

### Synthesis of Compound 140

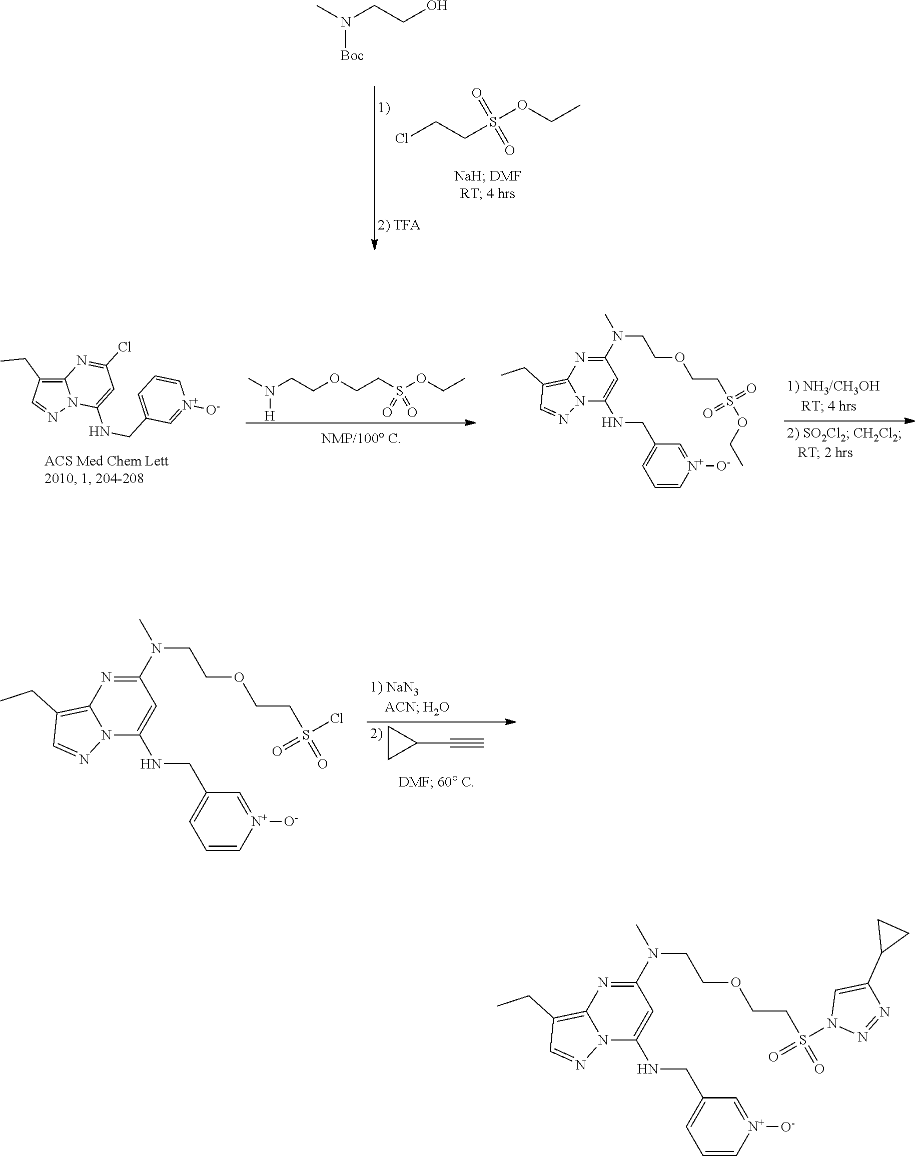

### Synthesis of Compound 141

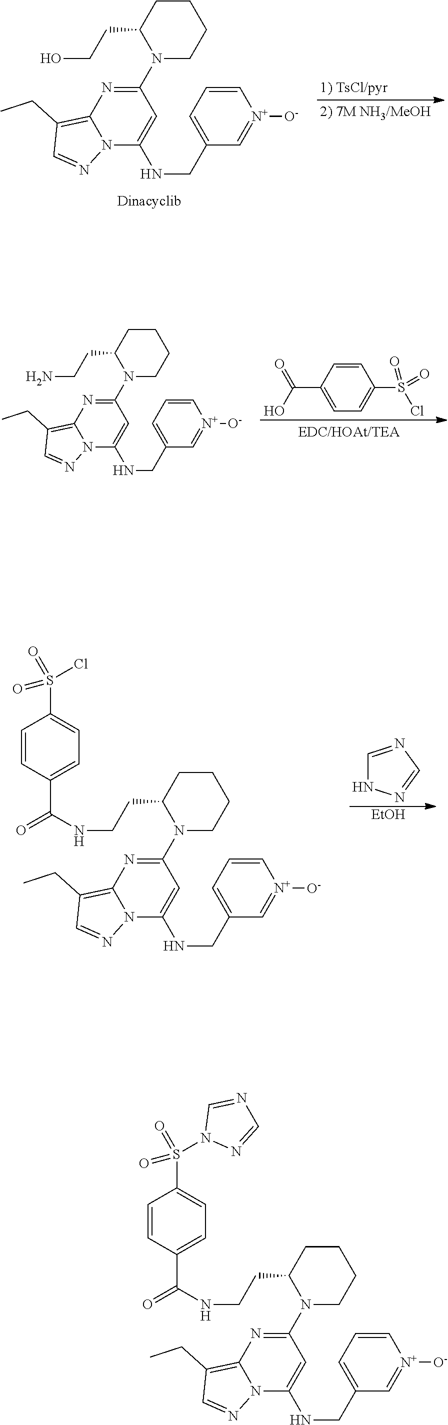

### Synthesis of Compound 142

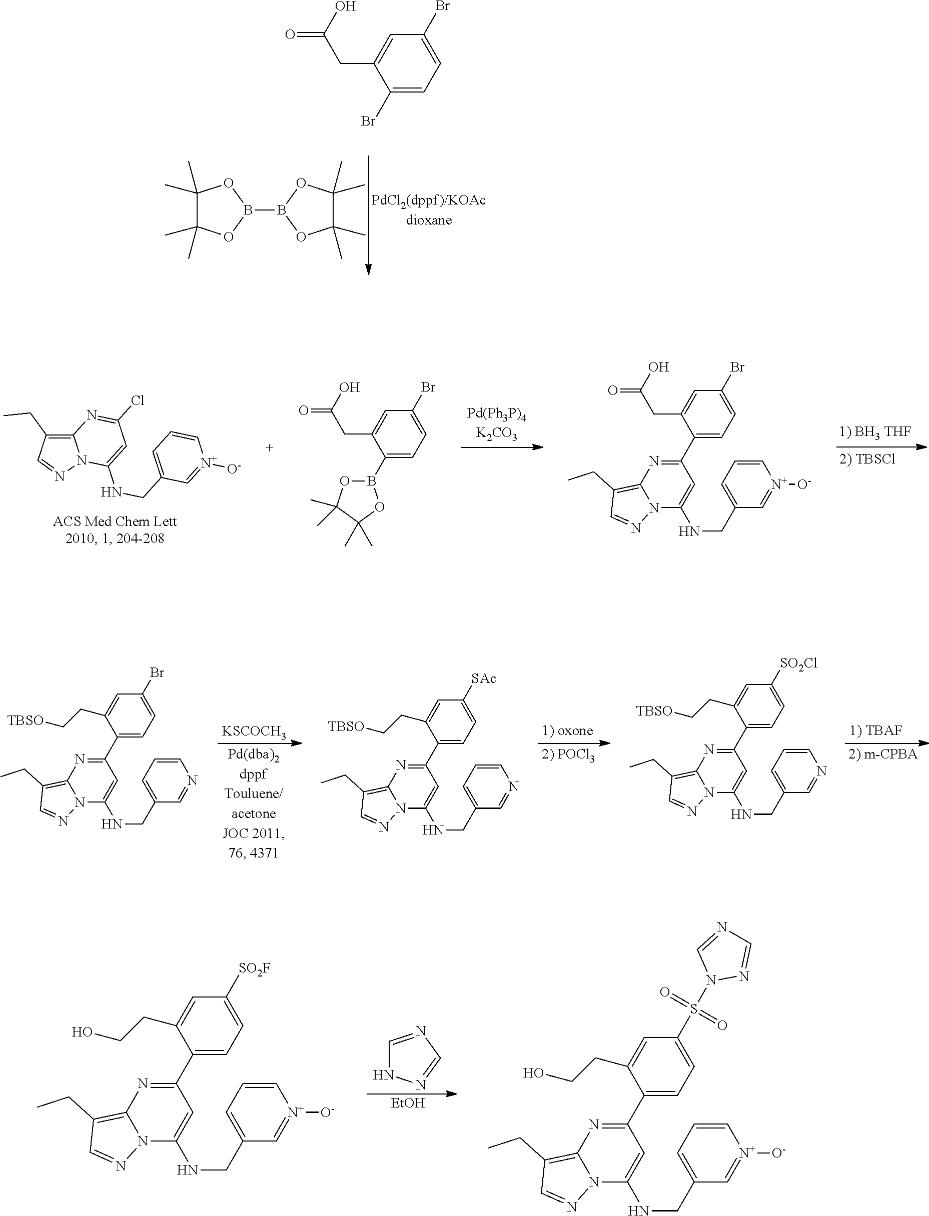

### Synthesis of Compound 143

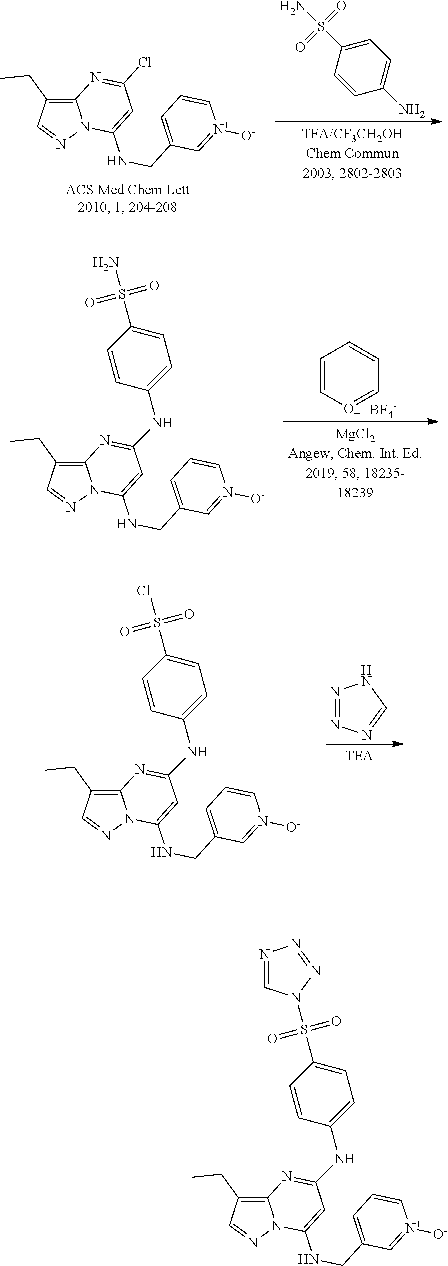

### Synthesis of Compound 144

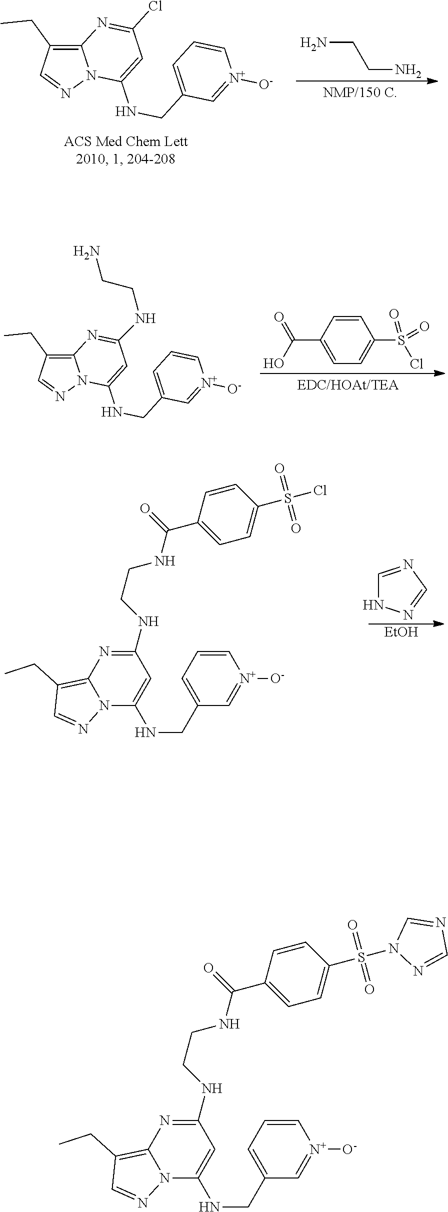

### Synthesis of Compound 145

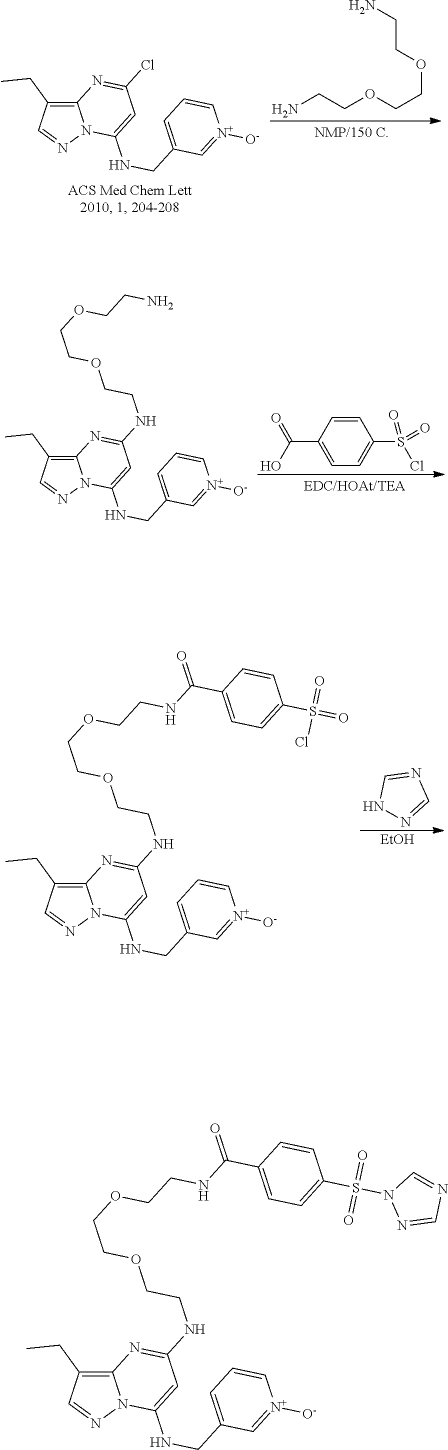

### Synthesis of Compound 146

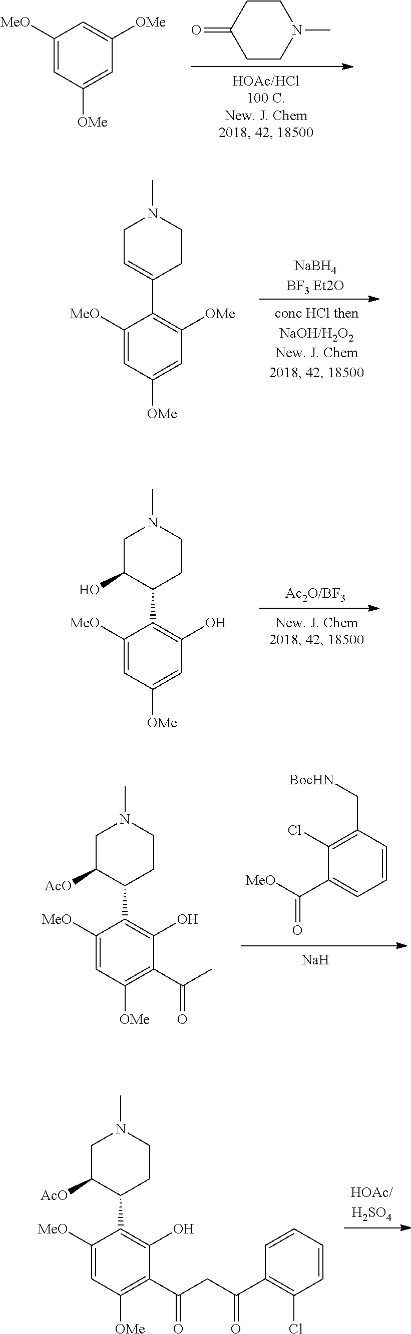

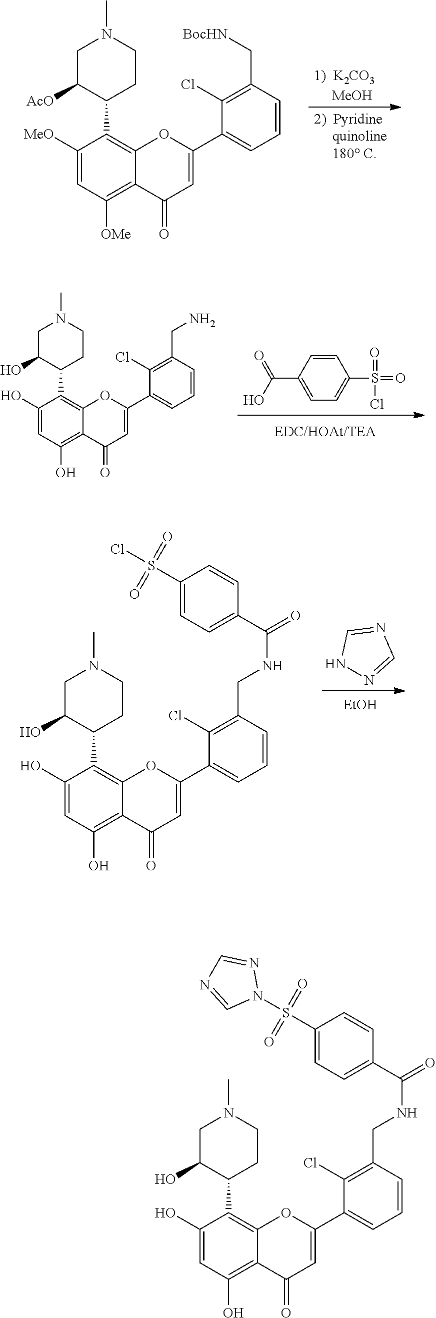

### Synthesis of Compound 147

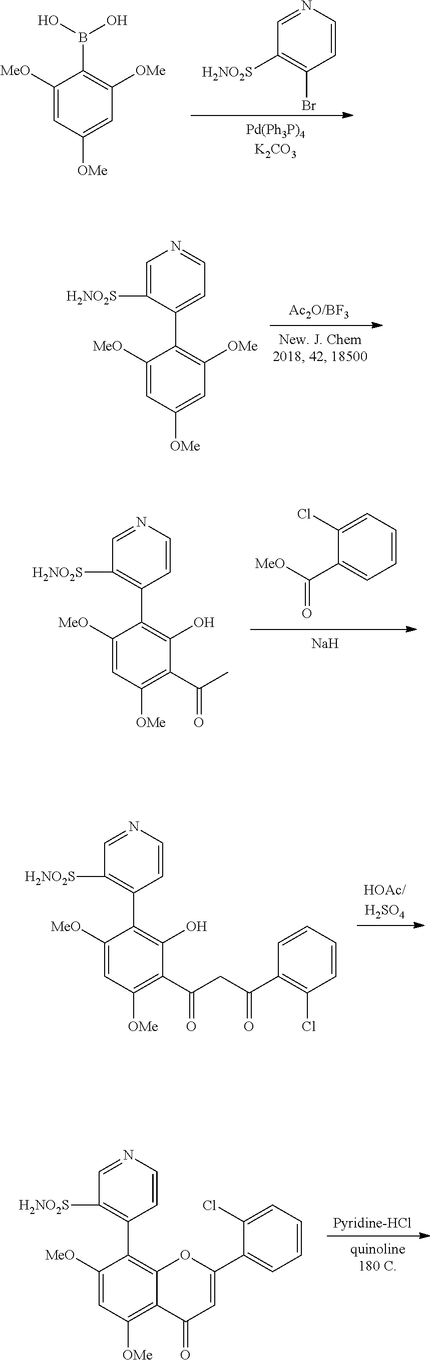

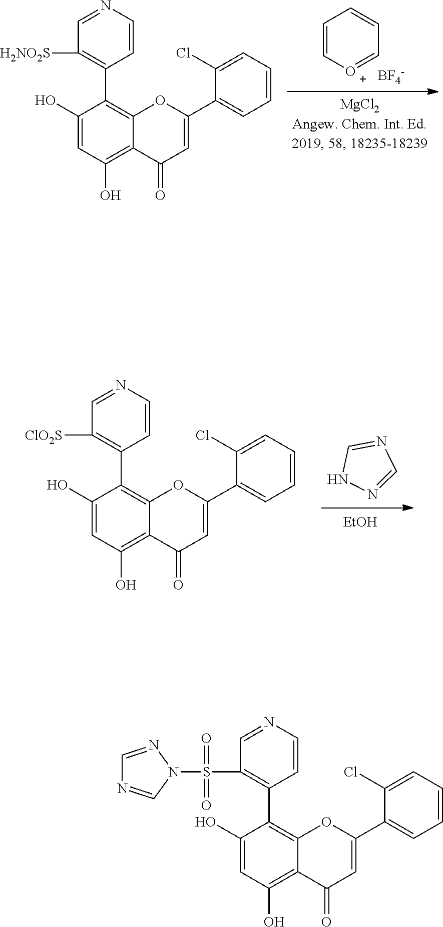

### Synthesis of Compound 148

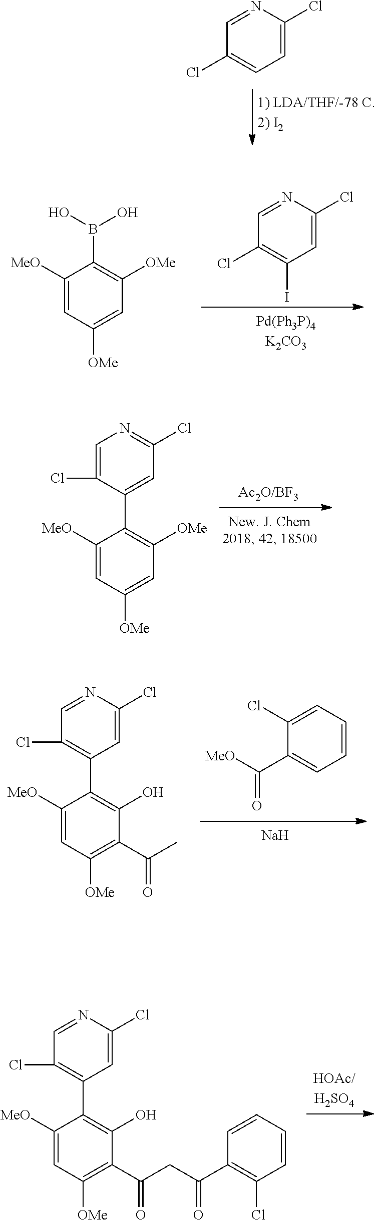

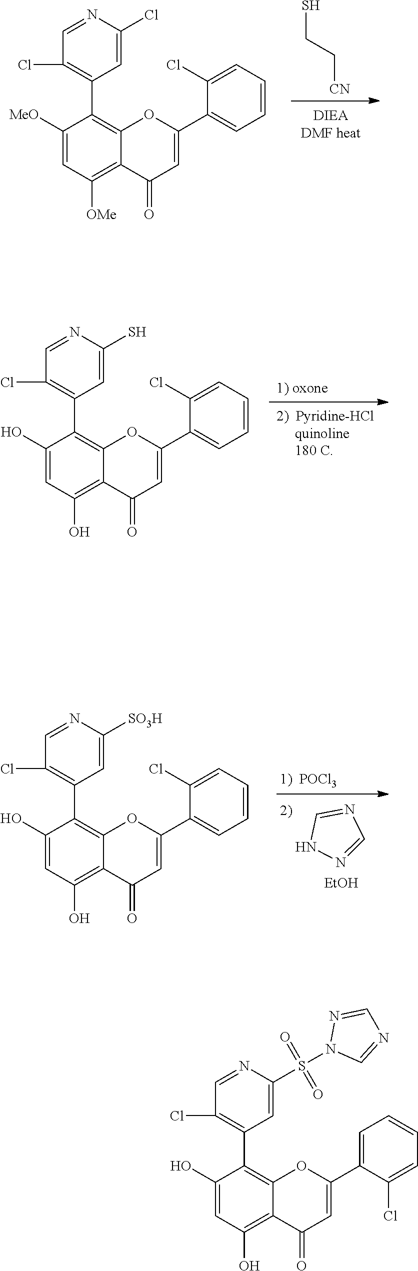

### Synthesis of Corn und 149

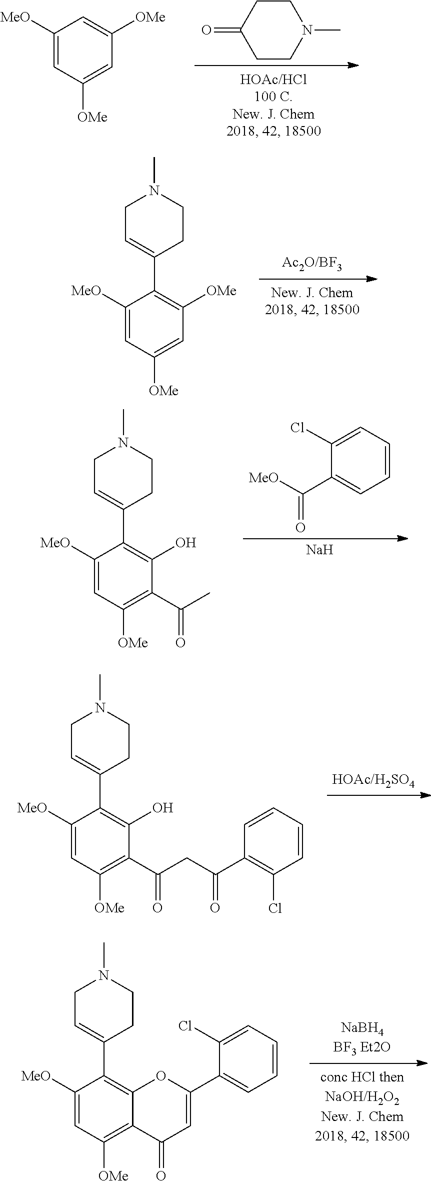

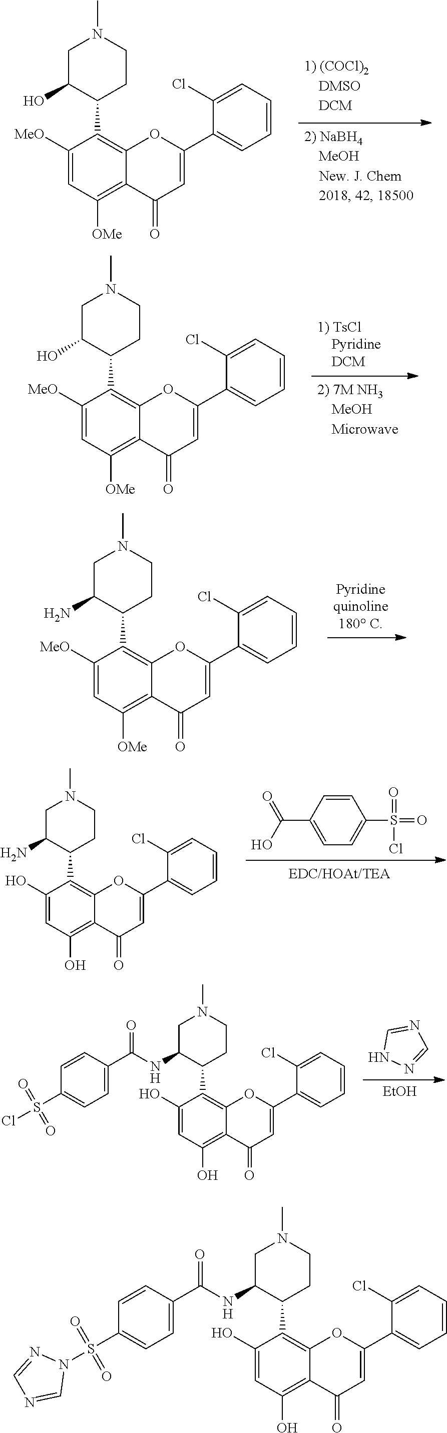

### Synthesis of Compound 150

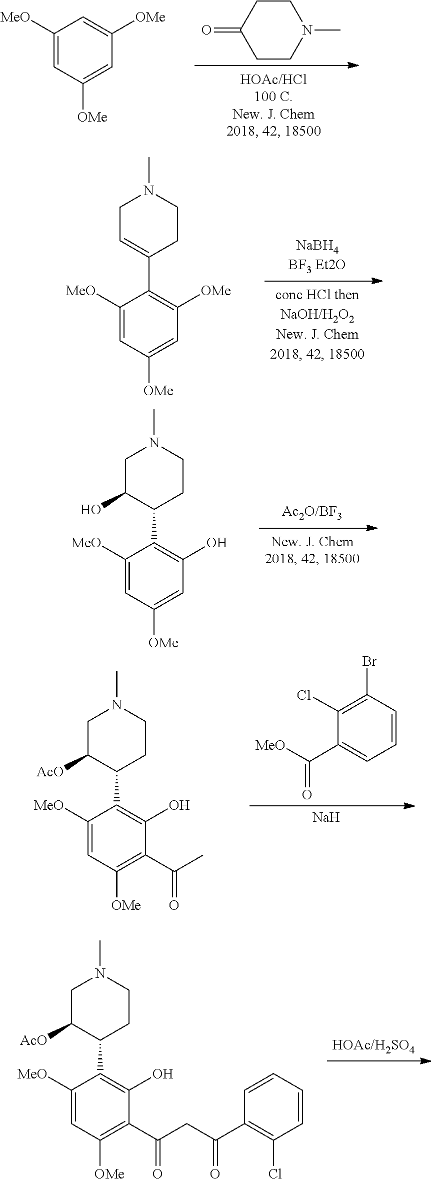

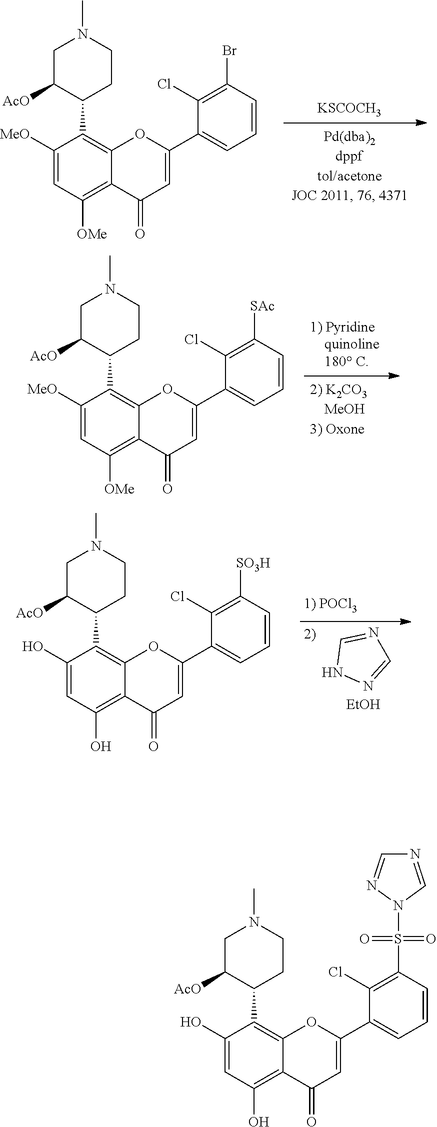

### Synthesis of Compound 151

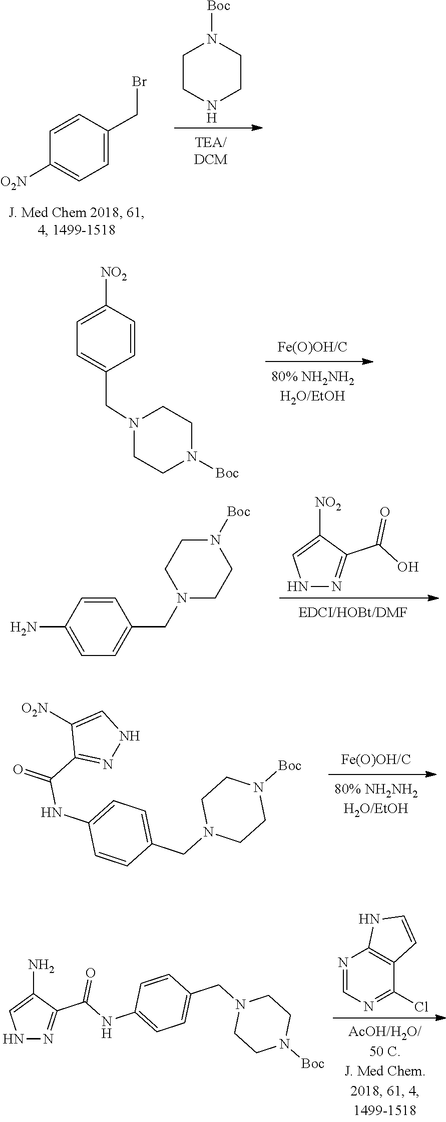

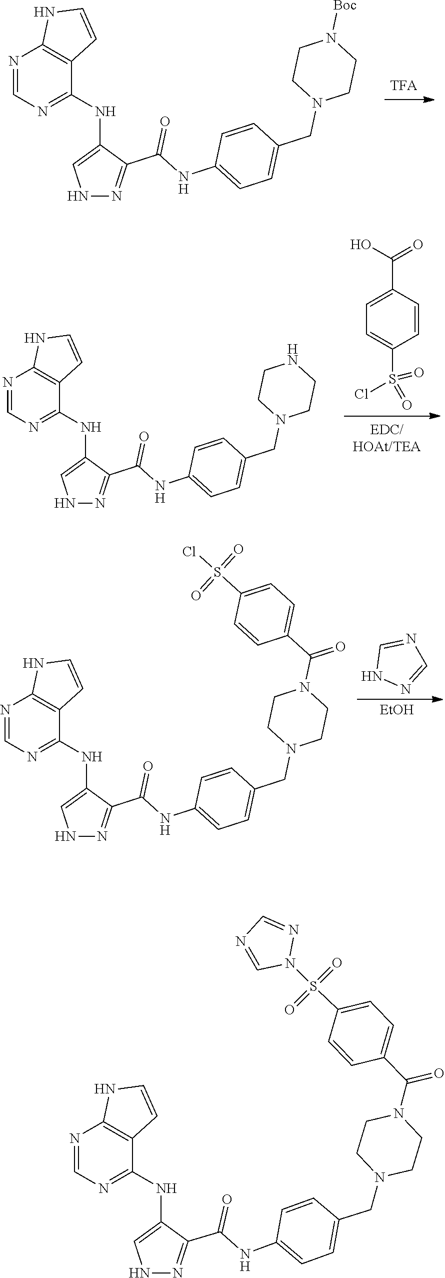

### Synthesis of Compound 152

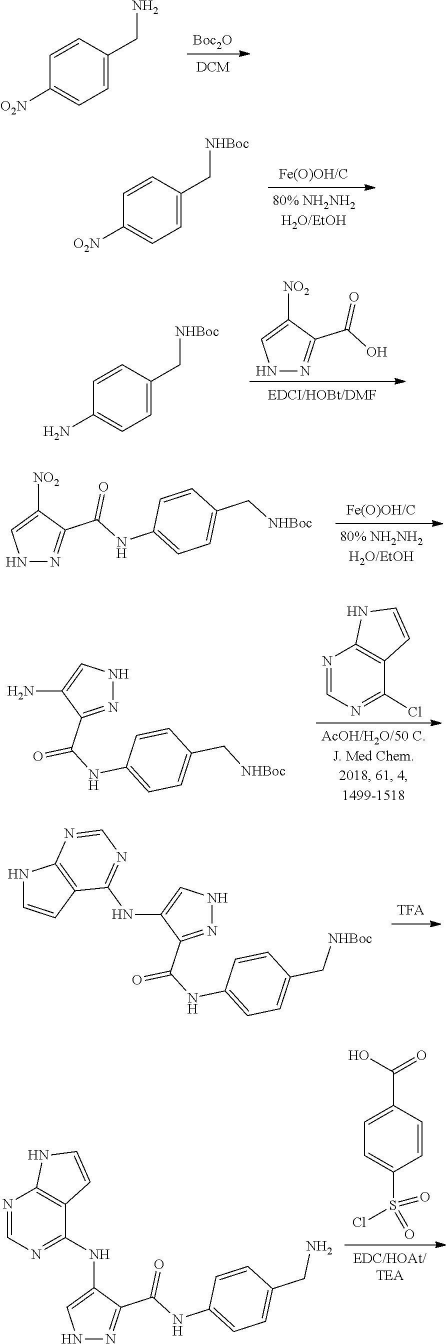

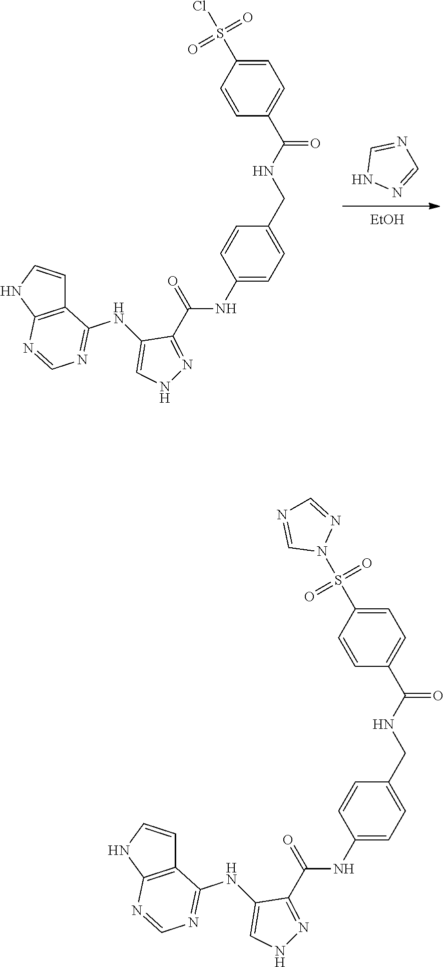 [SMILES: "EtOH" ; O=C(NCc1ccc(NC(=O)c2n[nH]cc2Nc2ncnc3[nH]ccc23)cc1)c1ccc(S(=O)(=O)Cl)cc1 ; c1nc[nH]n1 ; O=C(NCc1ccc(NC(=O)c2n[nH]cc2Nc2ncnc3[nH]ccc23)cc1)c1ccc(S(=O)(=O)n2cncn2)cc1]

### Synthesis of Compound 153

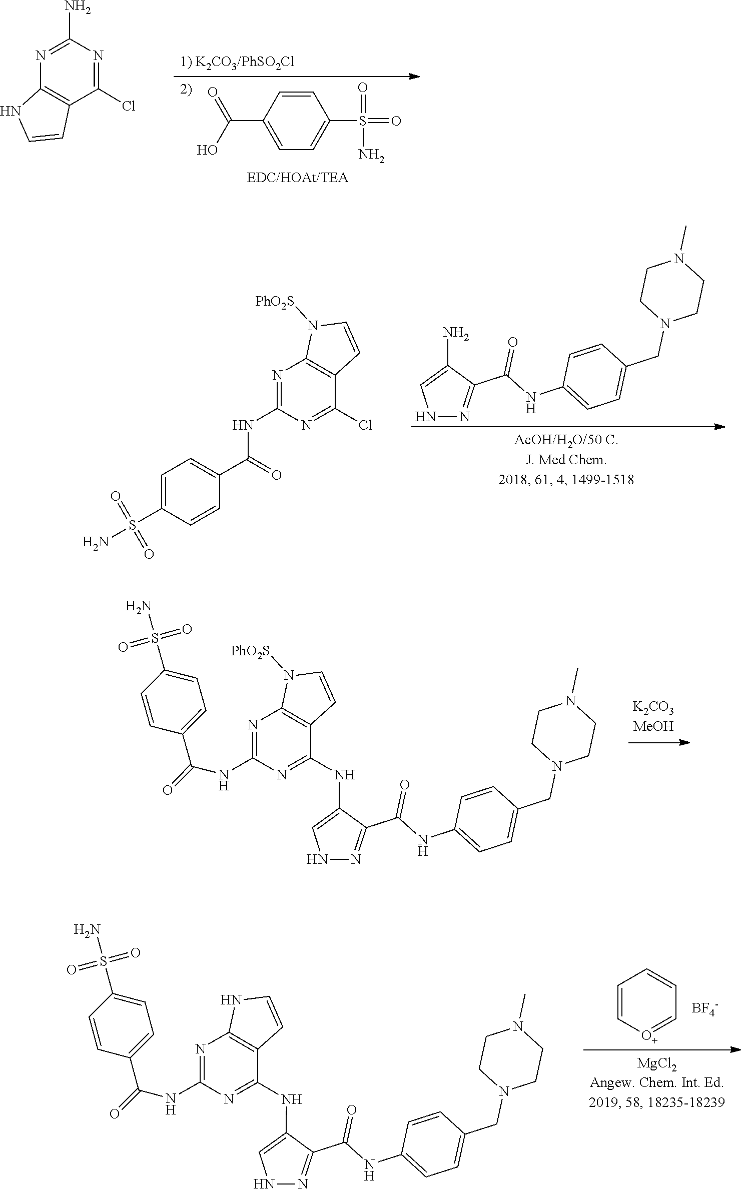

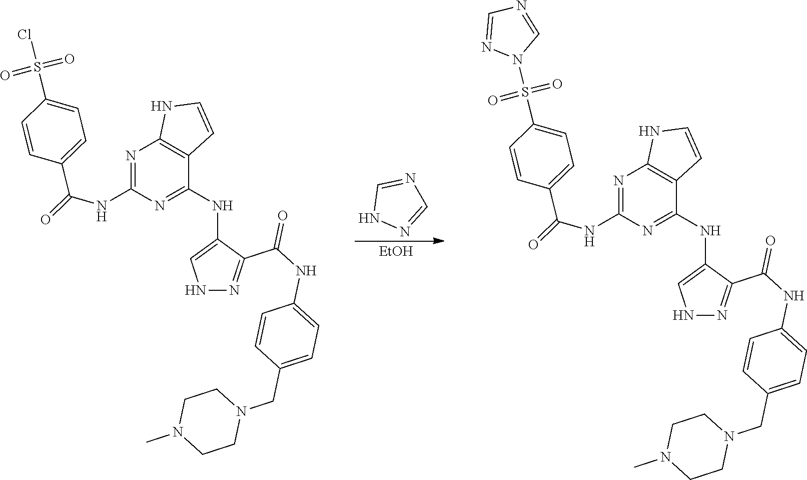 [SMILES: "EtOH" ; CN1CCN(Cc2ccc(NC(=O)c3n[nH]cc3Nc3nc(NC(=O)c4ccc(S(=O)(=O)Cl)cc4)nc4[nH]ccc34)cc2)CC1 ; c1nc[nH]n1 ; CN1CCN(Cc2ccc(NC(=O)c3n[nH]cc3Nc3nc(NC(=O)c4ccc(S(=O)(=O)n5cncn5)cc4)nc4[nH]ccc34)cc2)CC1]

### Synthesis of Compound 154

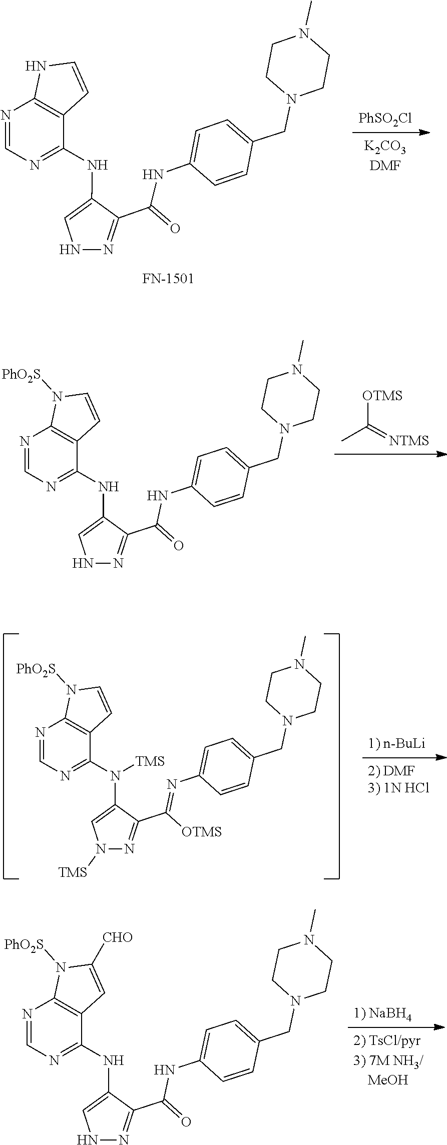

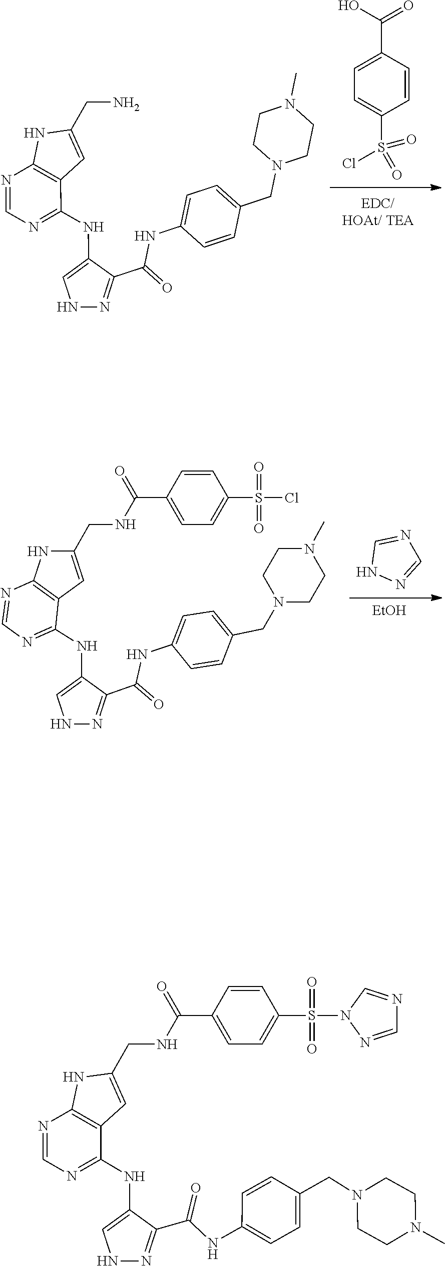

### Synthesis of Compound 155

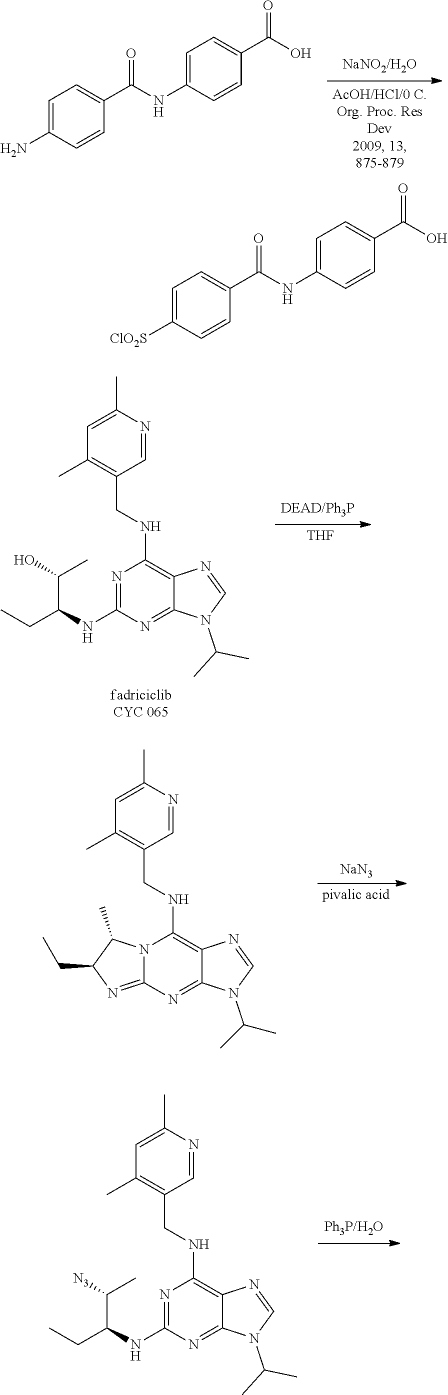

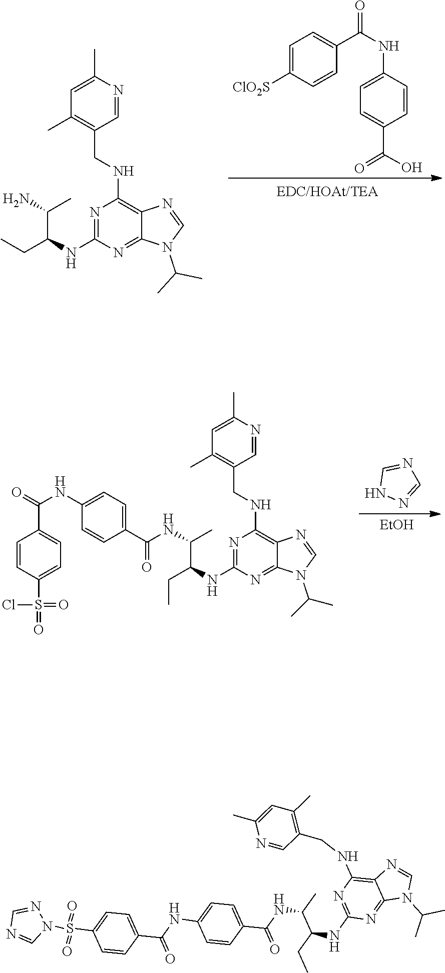

### Synthesis of Compound 156

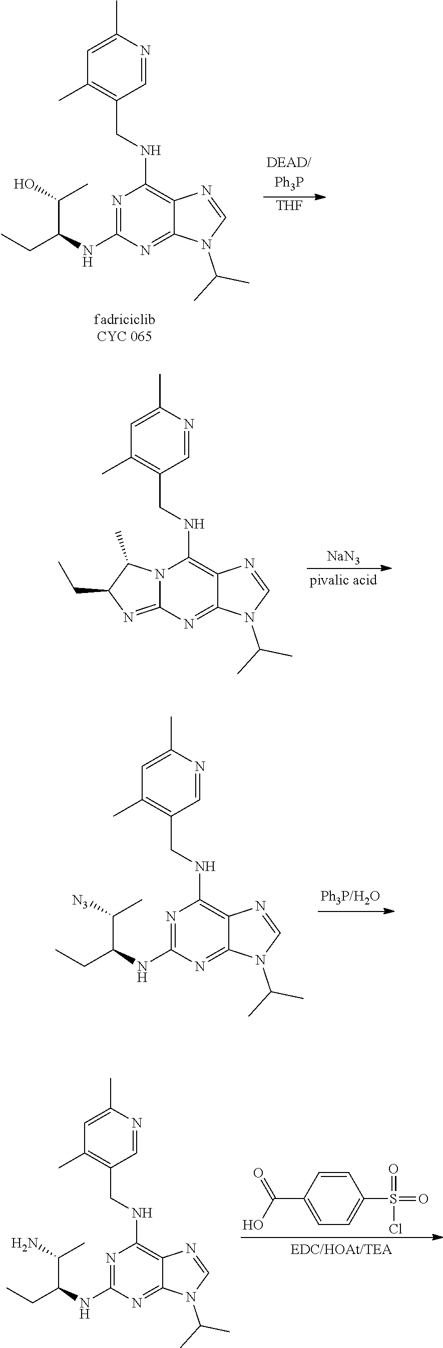

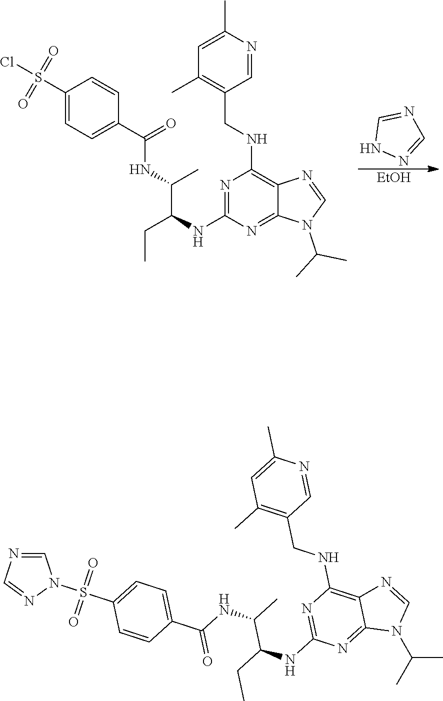 [SMILES: "EtOH" ; CC[C@H](Nc1nc(NCc2cnc(C)cc2C)c2ncn(C(C)C)c2n1)[C@@H](C)NC(=O)c1ccc(S(=O)(=O)Cl)cc1 ; c1nc[nH]n1 ; CC[C@H](Nc1nc(NCc2cnc(C)cc2C)c2ncn(C(C)C)c2n1)[C@@H](C)NC(=O)c1ccc(S(=O)(=O)n2cncn2)cc1]

### Synthesis of Compound 157

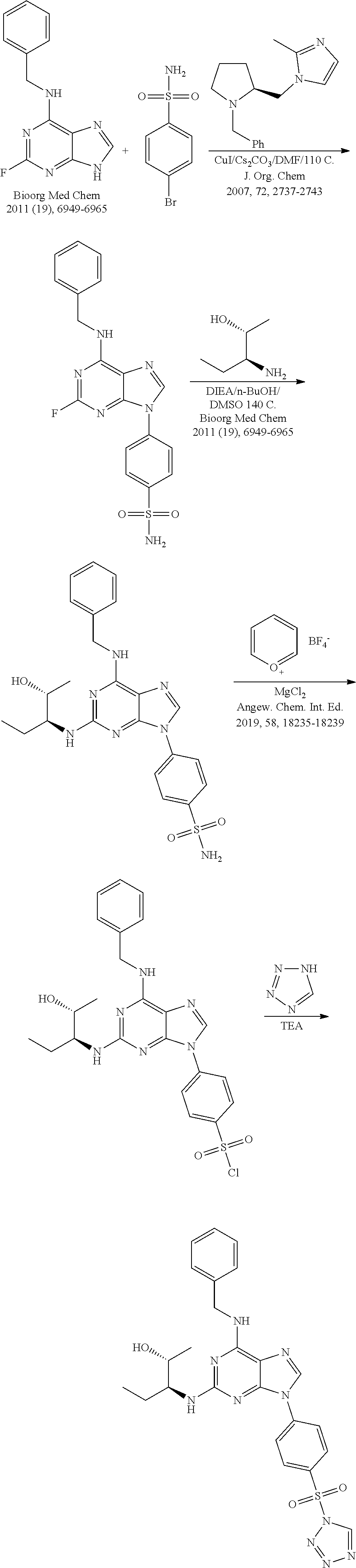

### Synthesis of Compound 158

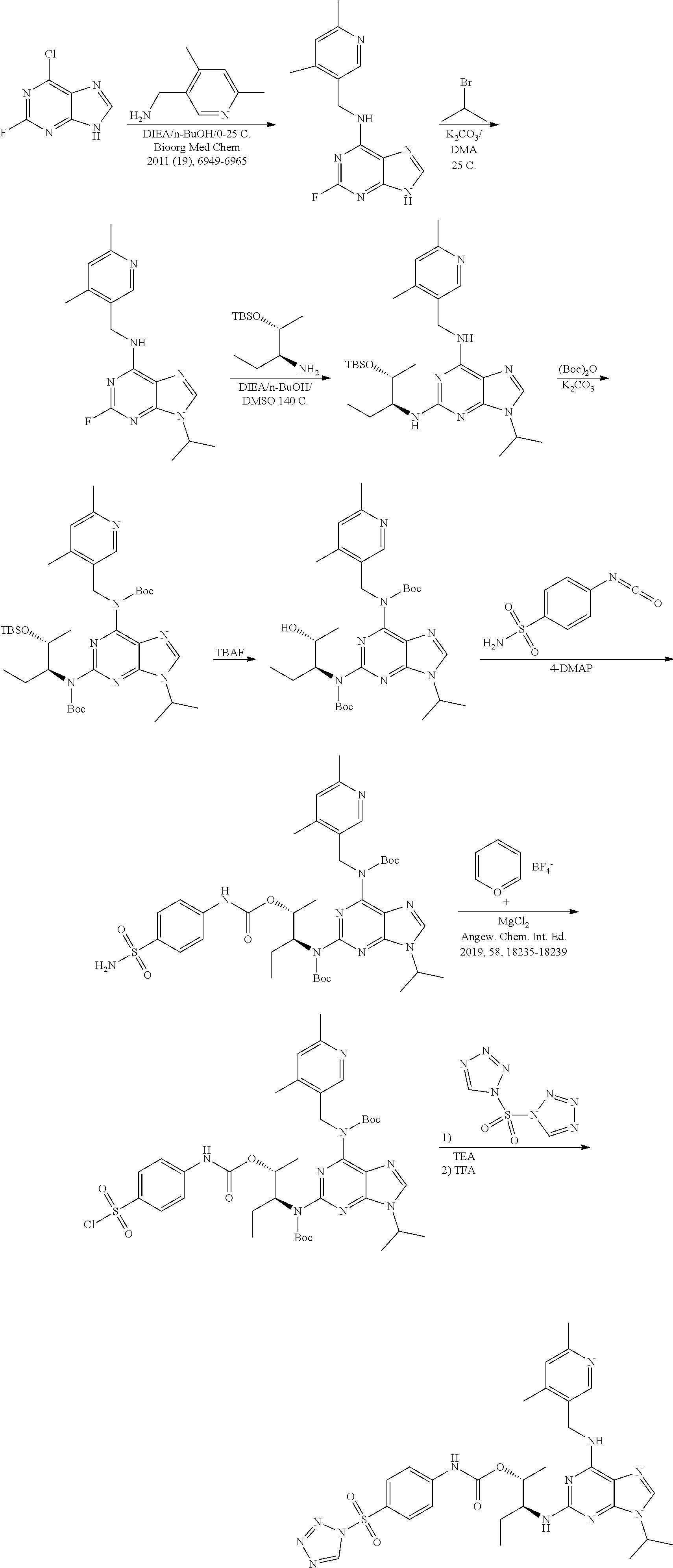

### Synthesis of Compound 159

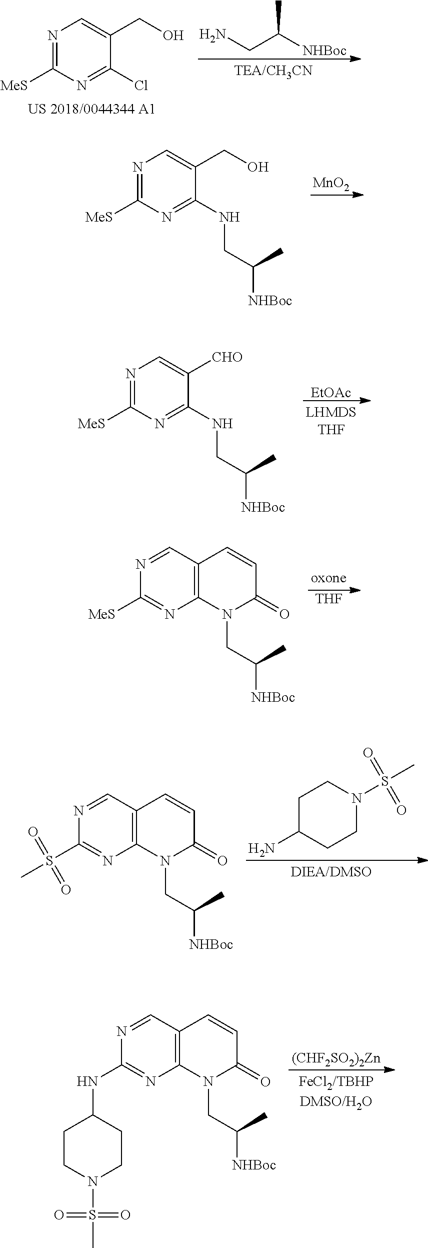

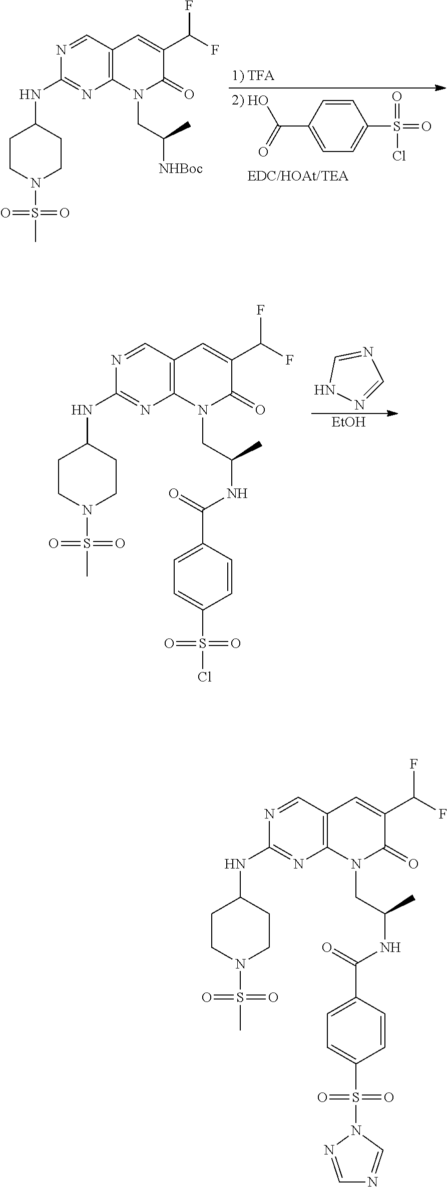

### Synthesis of Compound 160

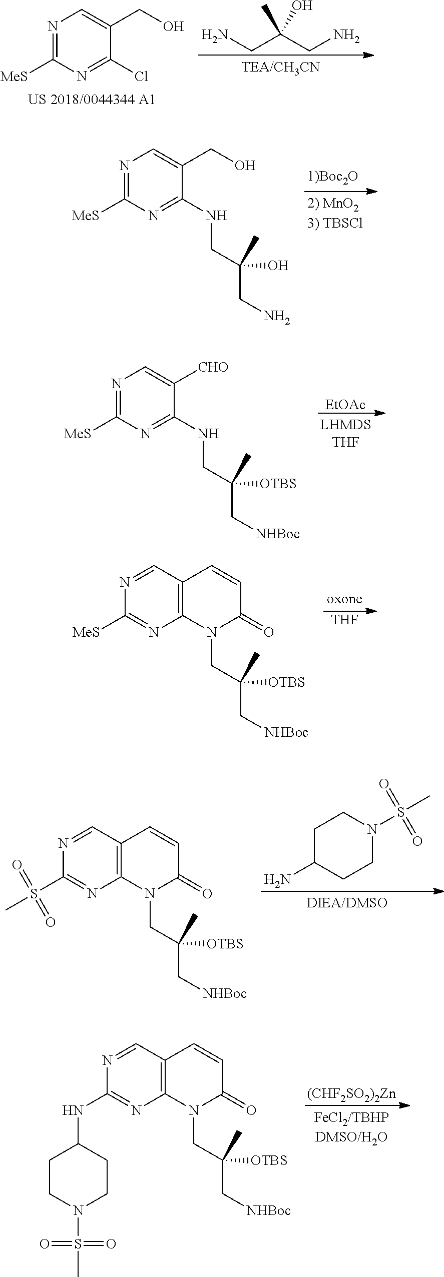

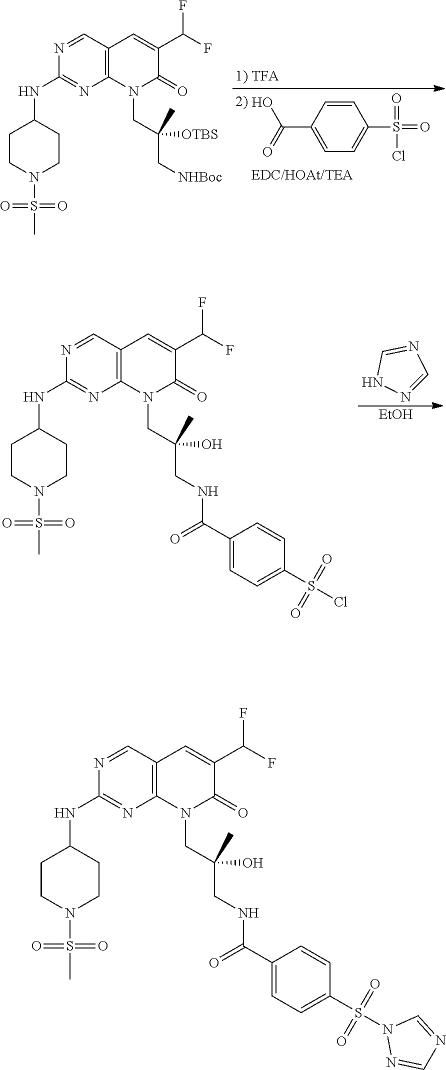

### Synthesis of Compound 161

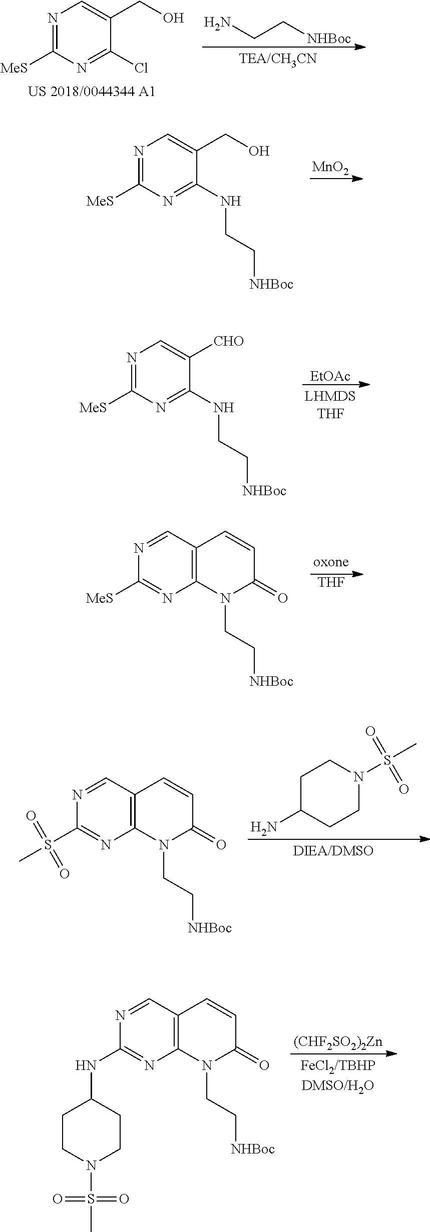

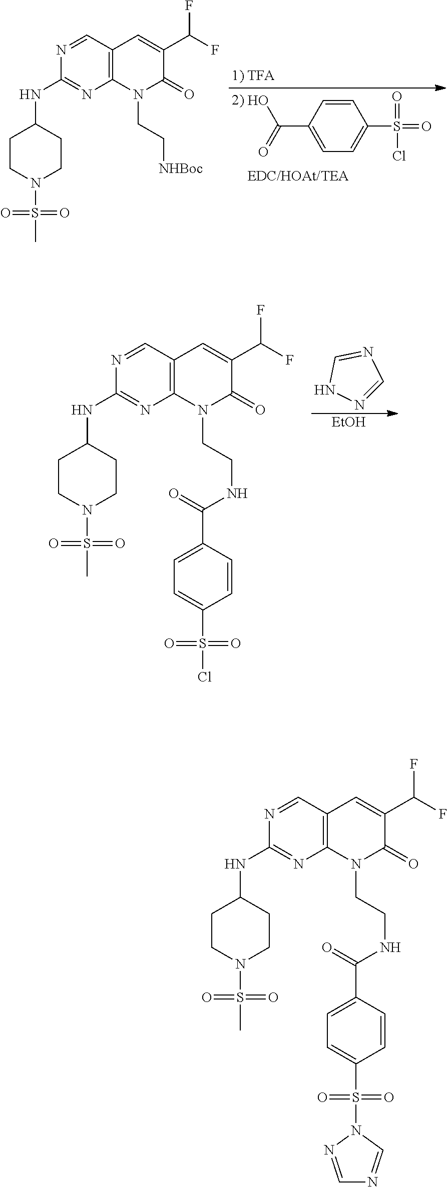

### Synthesis of Compound 162

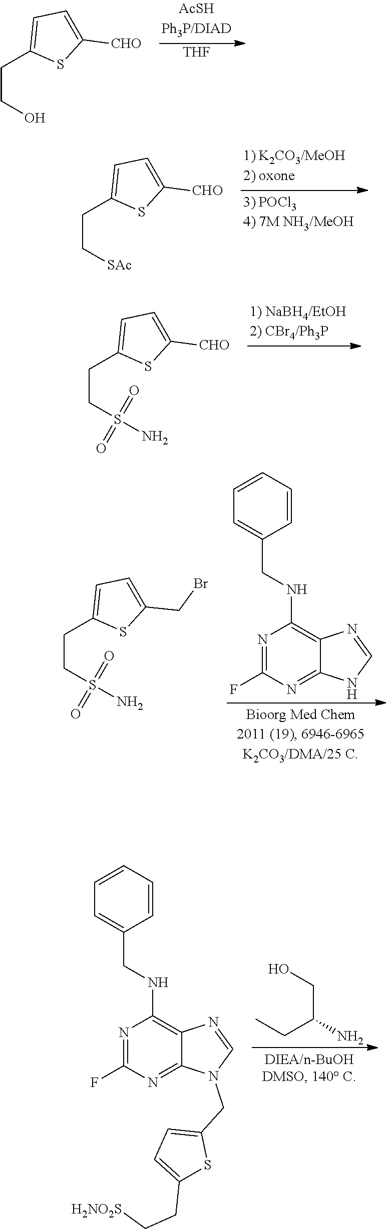

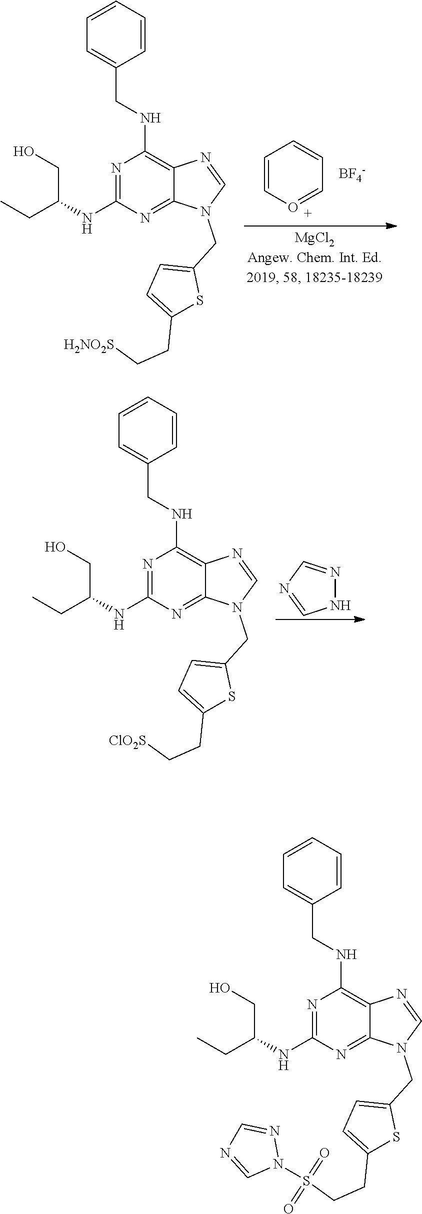

### Synthesis of Compound 163

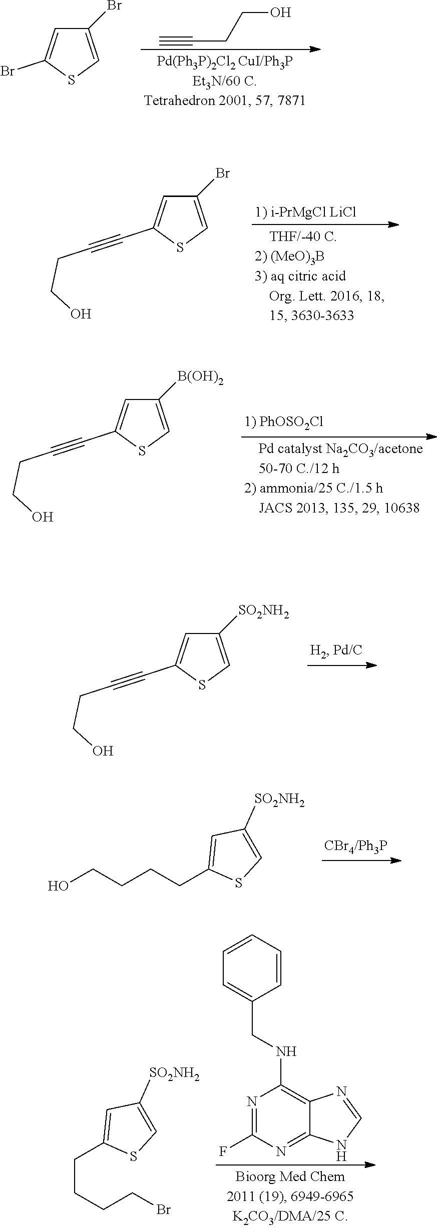

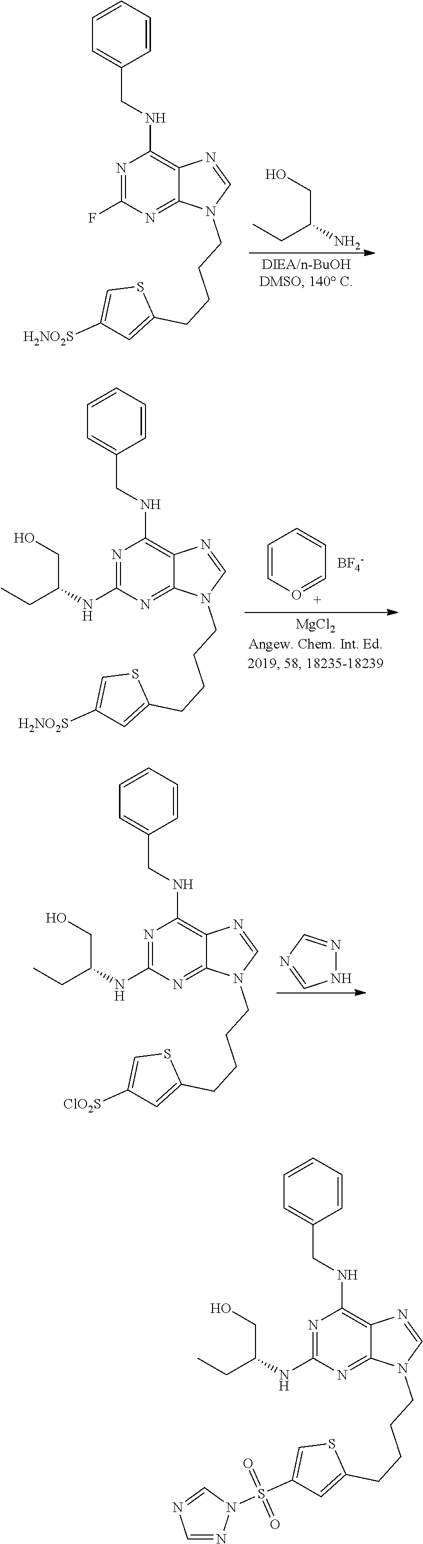

### Synthesis of Compound 164

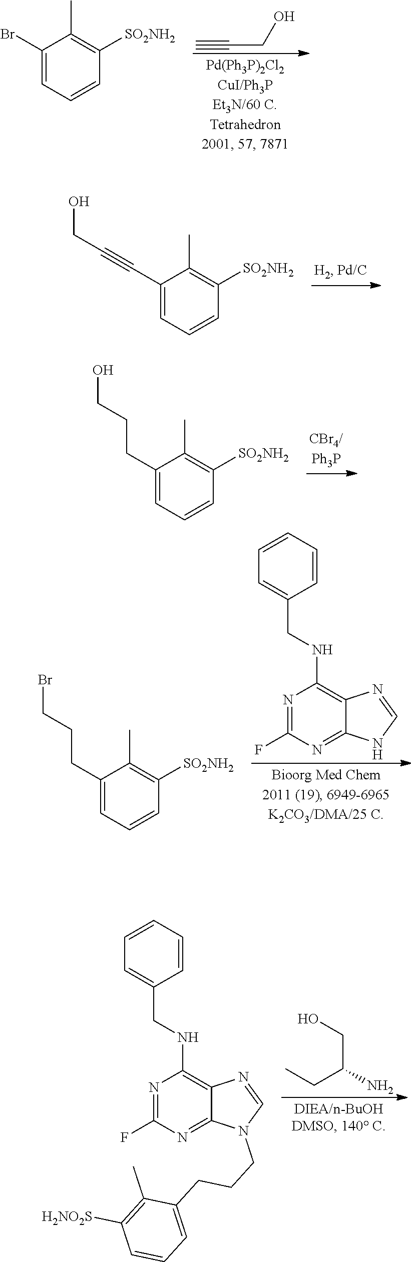

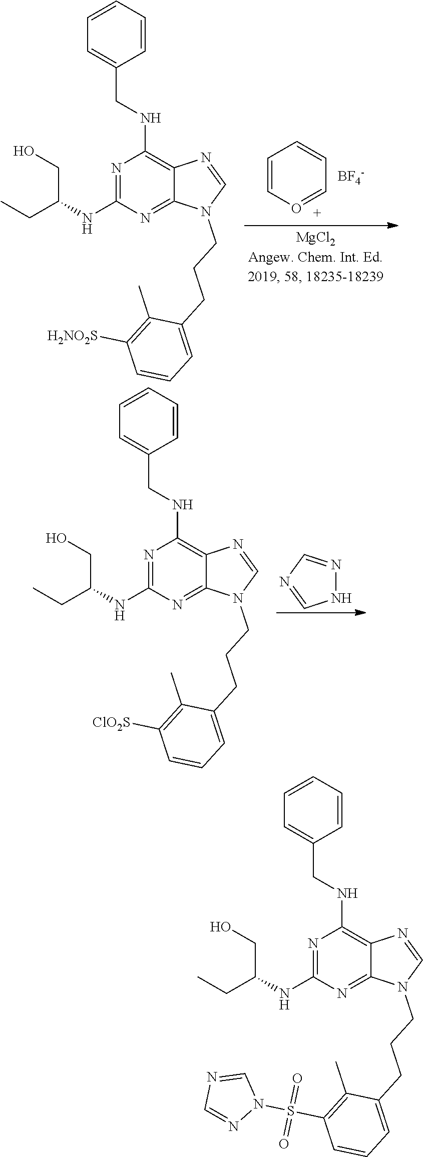

### Synthesis of Compound 165

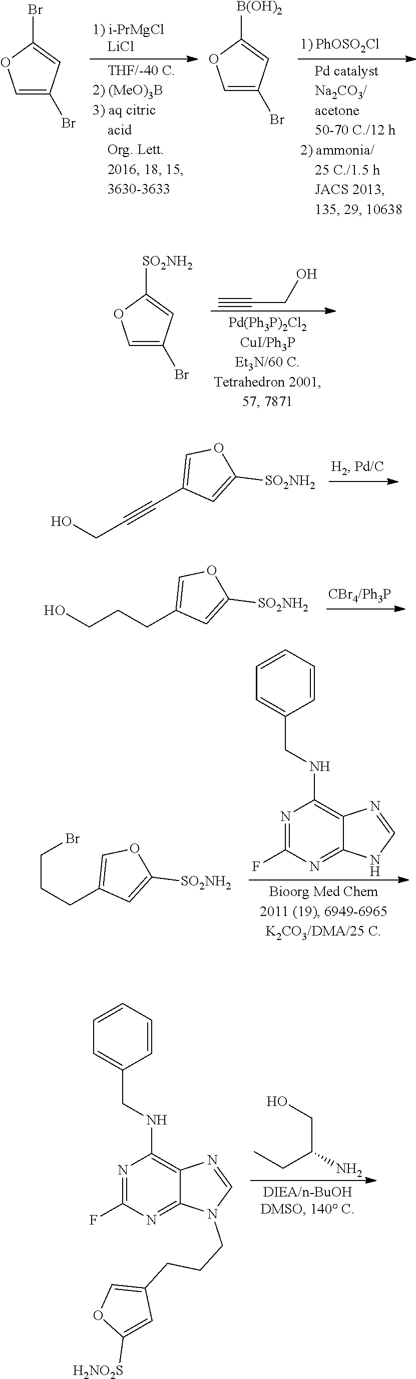

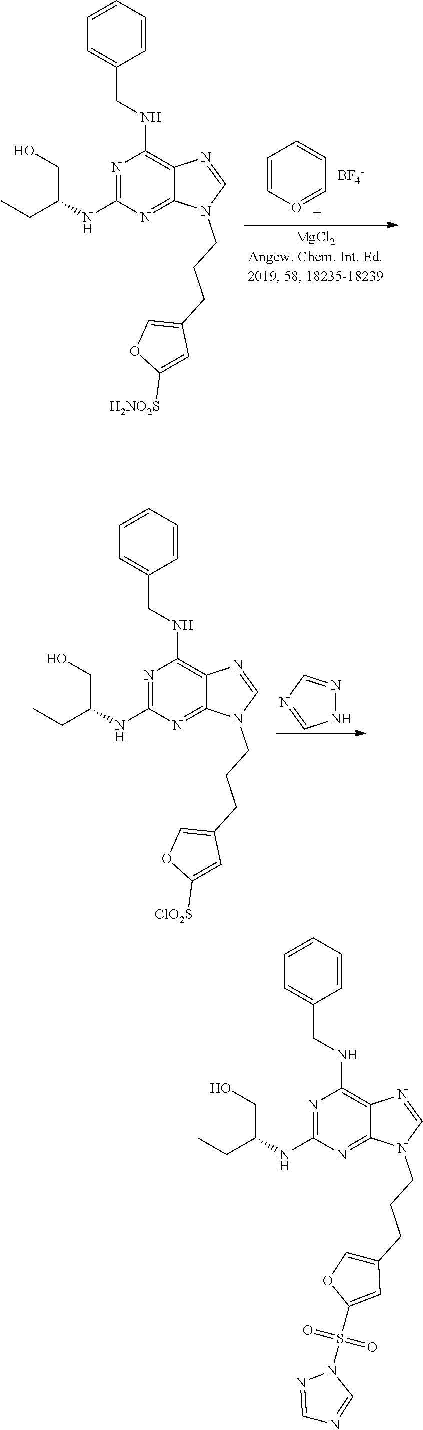

### Synthesis of Compound 166

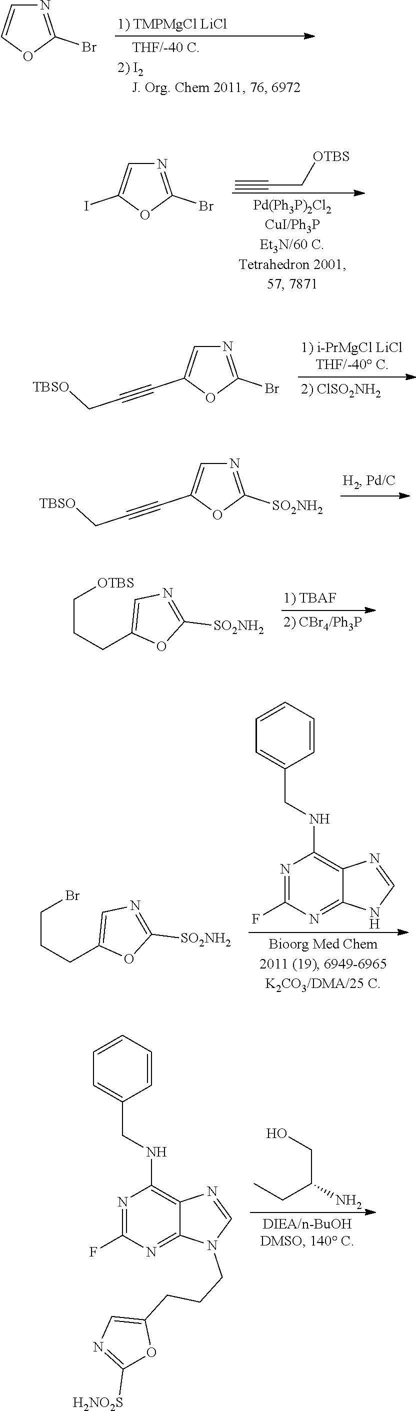

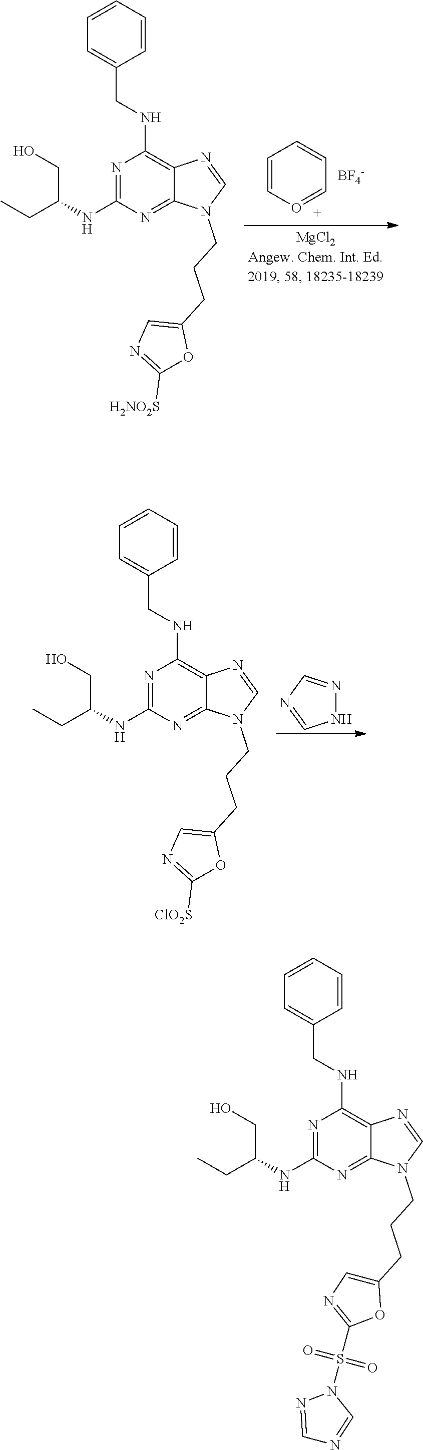

### Synthesis of Compound 167

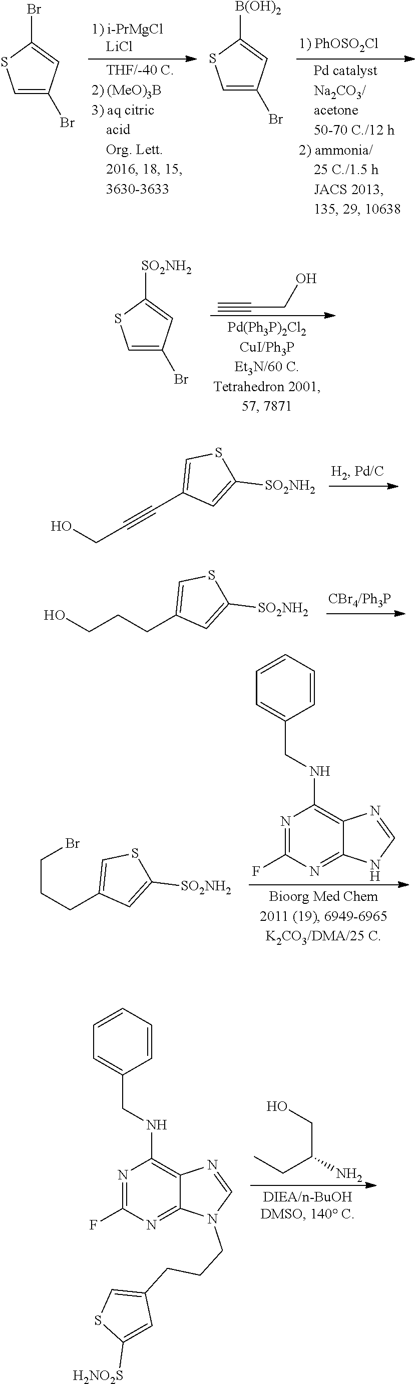

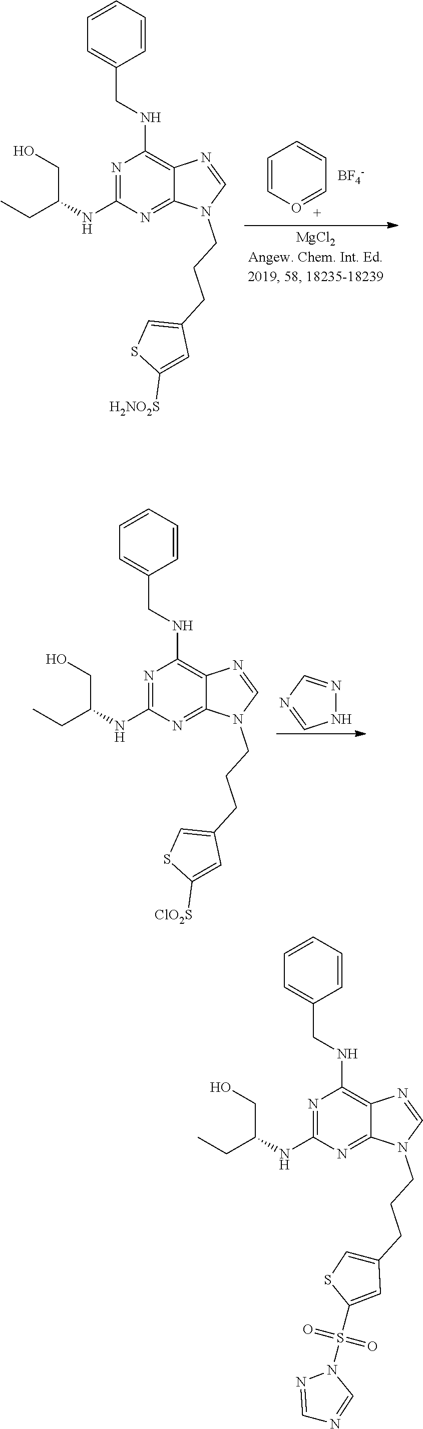

### Synthesis of Compound 168

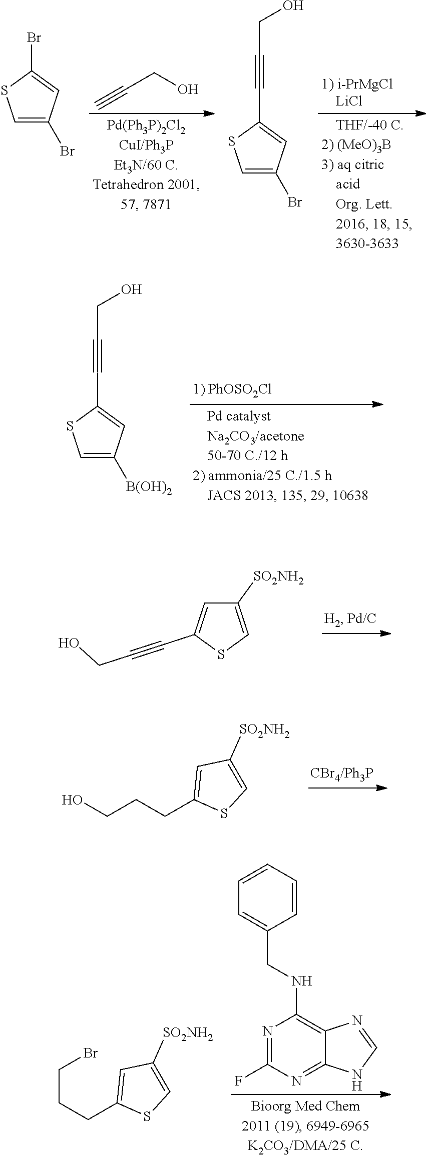

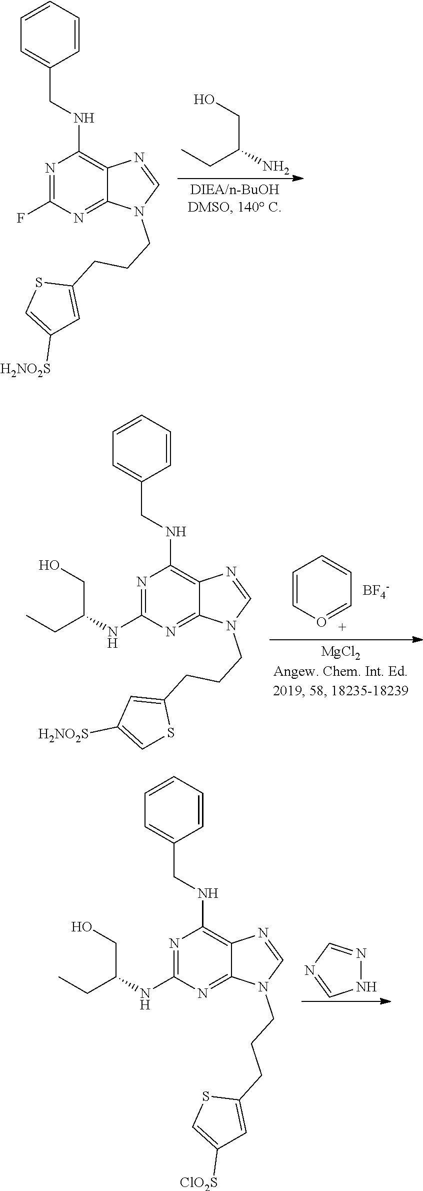

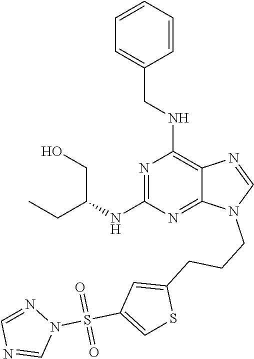 [SMILES: CC[C@H](CO)Nc1nc(NCc2ccccc2)c2ncn(CCCc3cc(S(=O)(=O)n4cncn4)cs3)c2n1]

### Synthesis of Compound 169

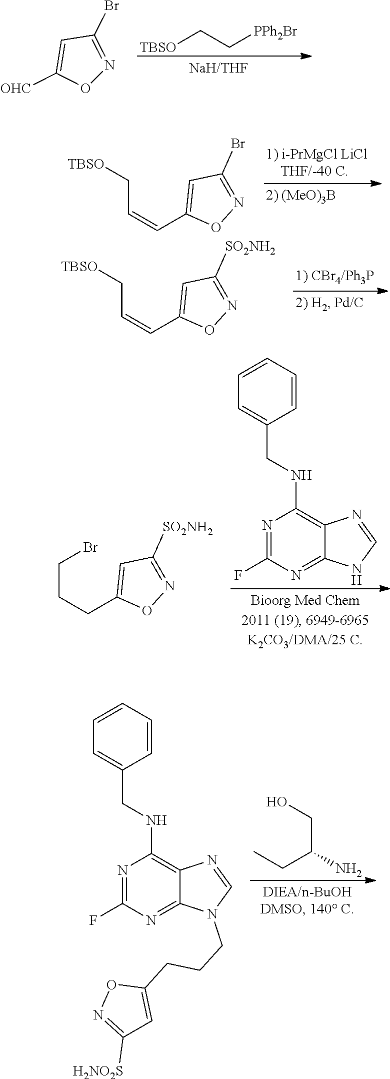

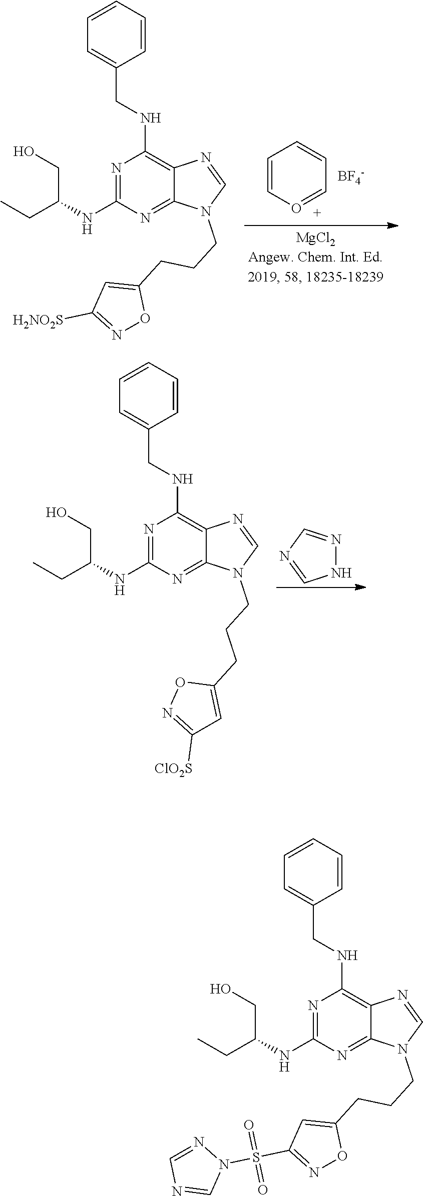

### Synthesis of Compound 170

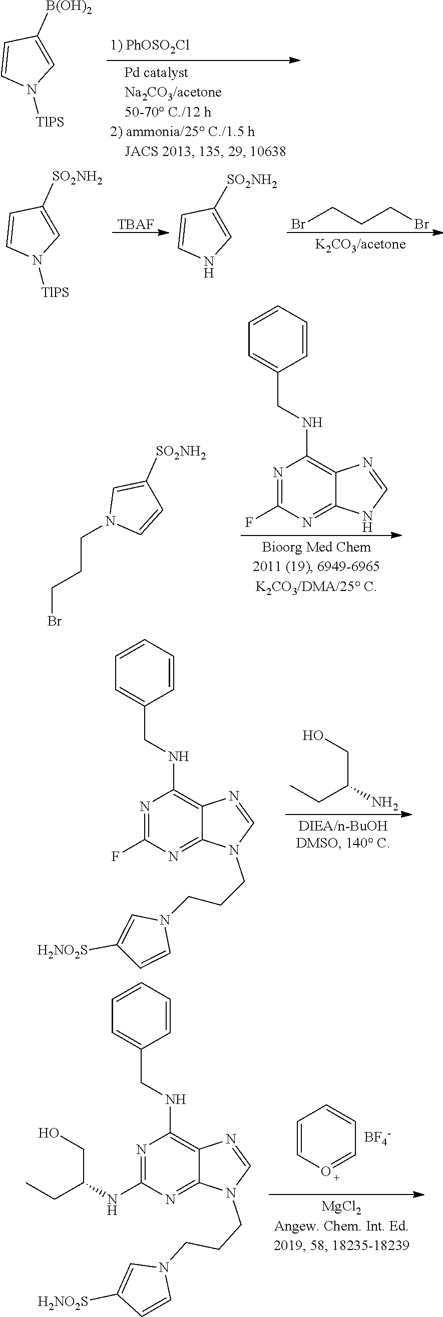

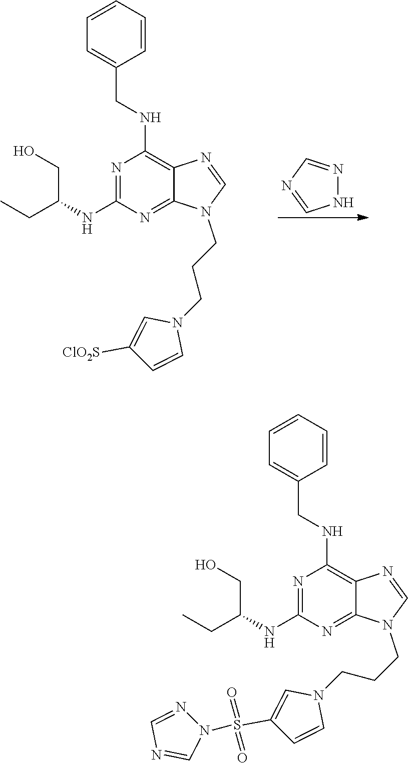 [SMILES: CC[C@H](CO)Nc1nc(NCc2ccccc2)c2ncn(CCCn3ccc(S(=O)(=O)Cl)c3)c2n1 ; c1nc[nH]n1 ; CC[C@H](CO)Nc1nc(NCc2ccccc2)c2ncn(CCCn3ccc(S(=O)(=O)n4cncn4)c3)c2n1]

### Synthesis of Compound 171

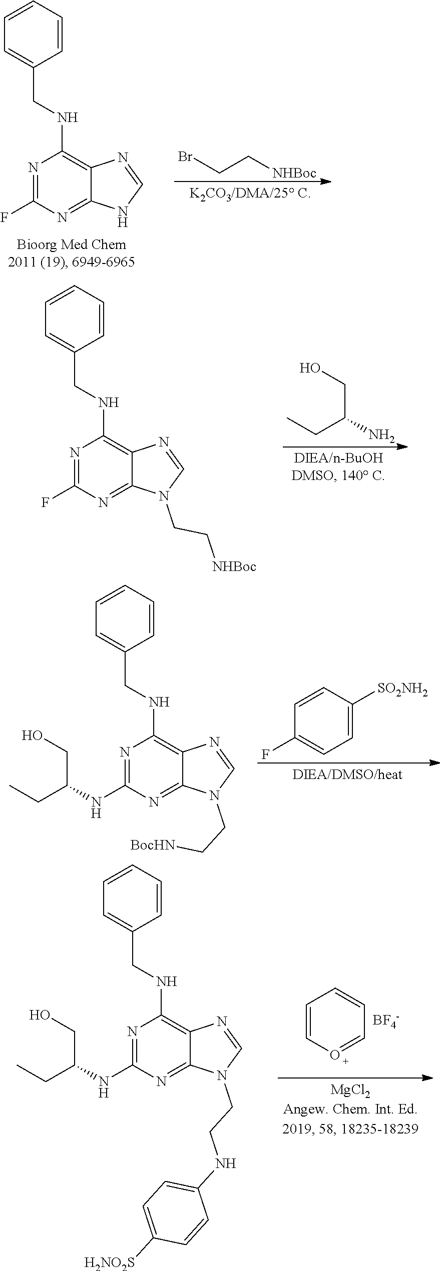

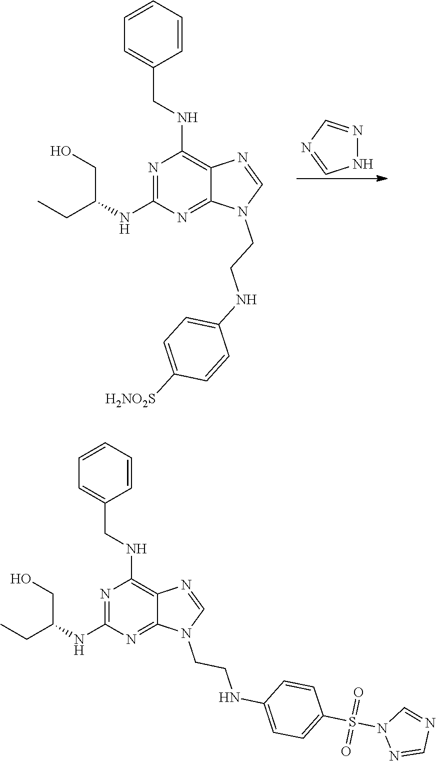

### Synthesis of Compound 172

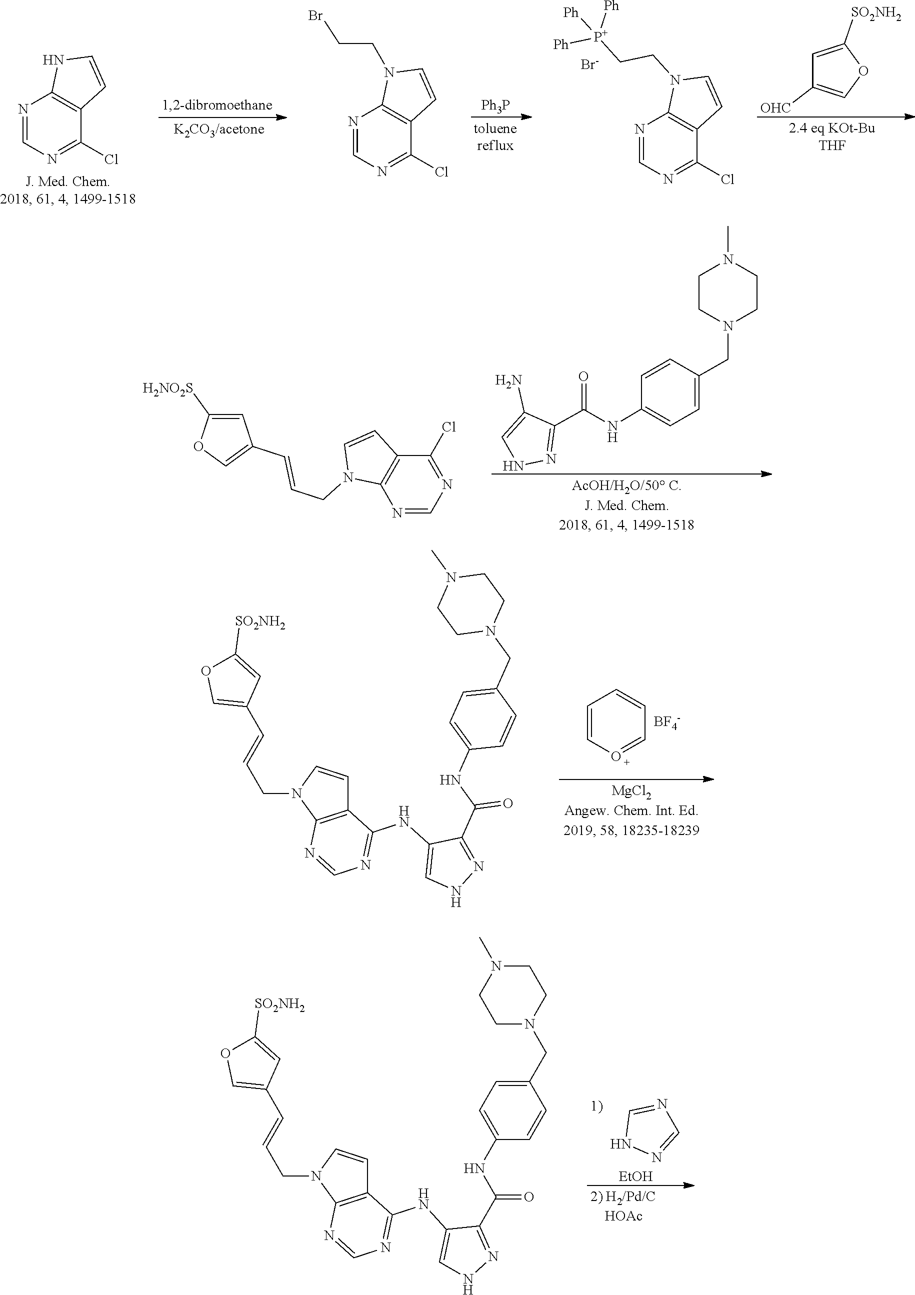

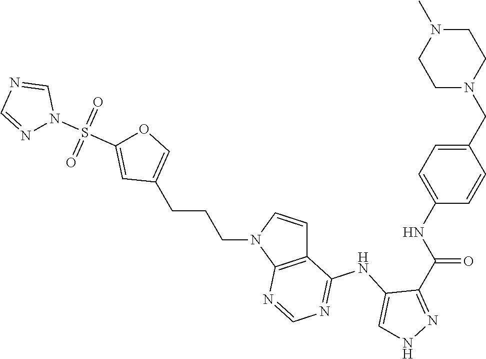 [SMILES: CN1CCN(Cc2ccc(NC(=O)c3n[nH]cc3Nc3ncnc4c3ccn4CCCc3coc(S(=O)(=O)n4cncn4)c3)cc2)CC1]

### Synthesis of Compound 173

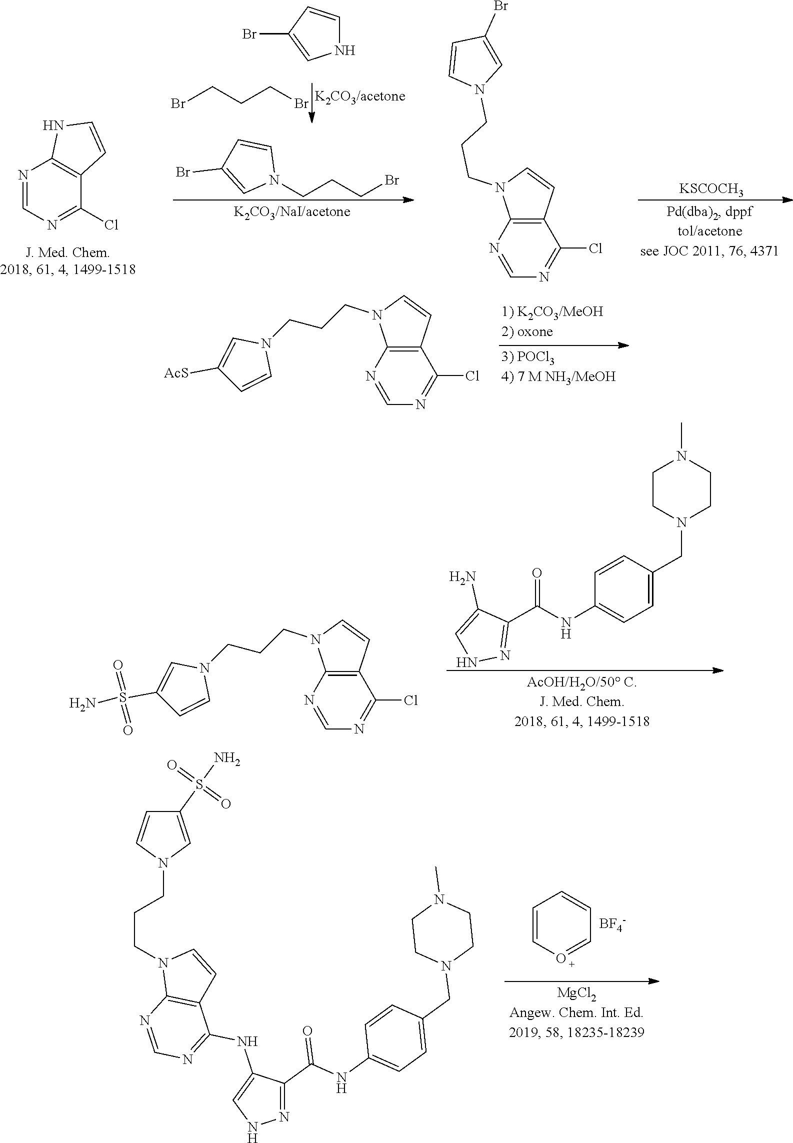

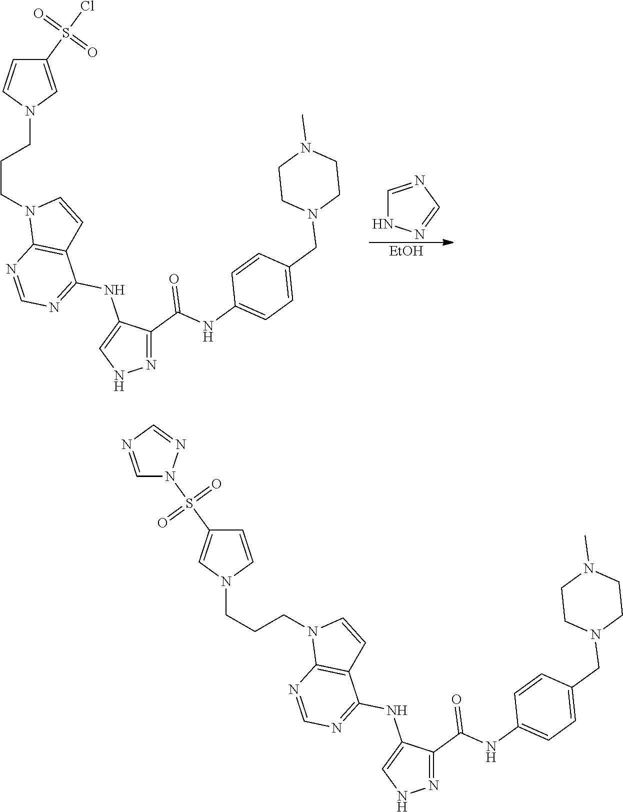 [SMILES: "EtOH" ; CN1CCN(Cc2ccc(NC(=O)c3n[nH]cc3Nc3ncnc4c3ccn4CCCn3ccc(S(=O)(=O)Cl)c3)cc2)CC1 ; c1nc[nH]n1 ; CN1CCN(Cc2ccc(NC(=O)c3n[nH]cc3Nc3ncnc4c3ccn4CCCn3ccc(S(=O)(=O)n4cncn4)c3)cc2)CC1]

### Synthesis of Compound 174

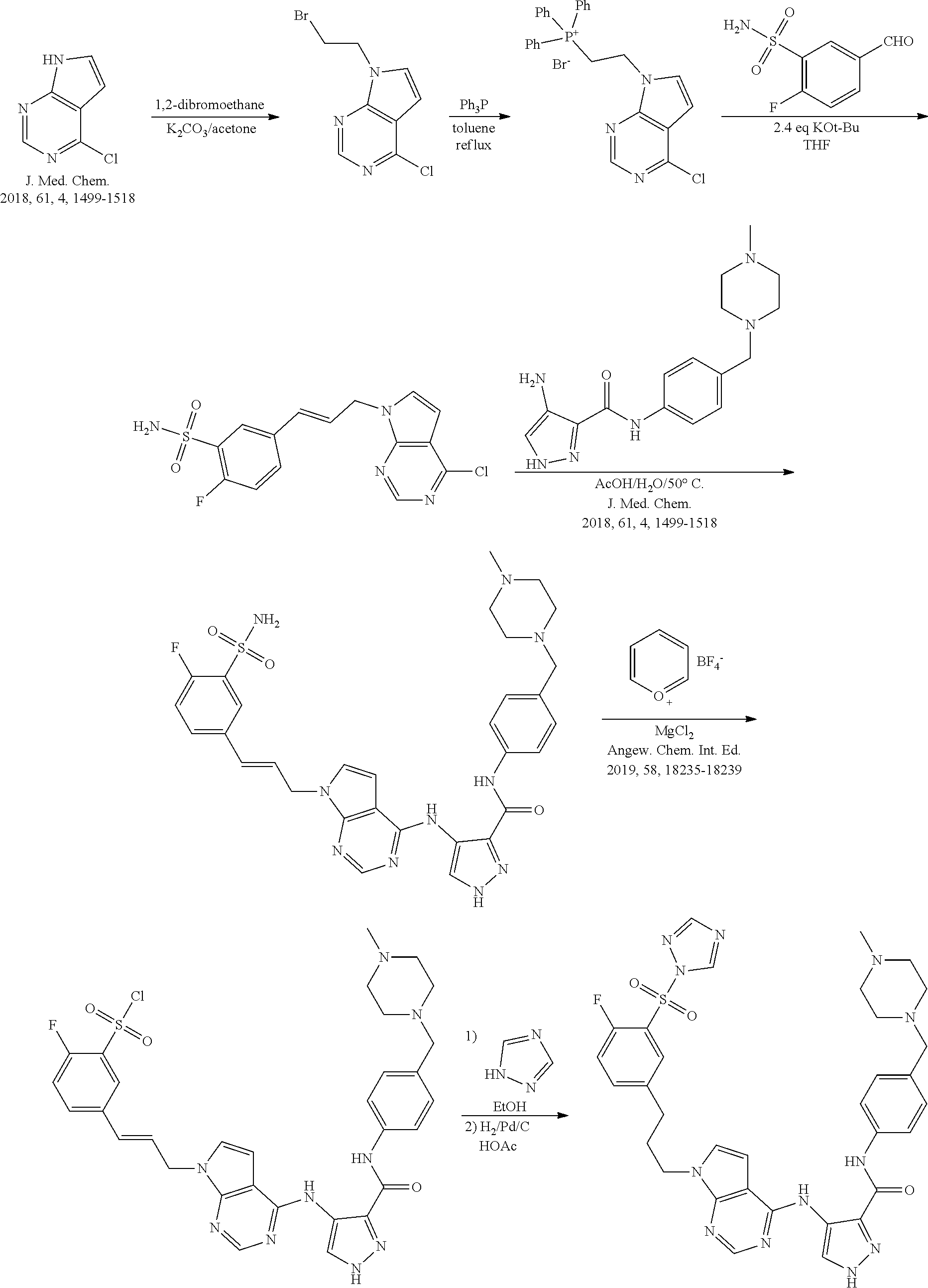

### Synthesis of Compound 175

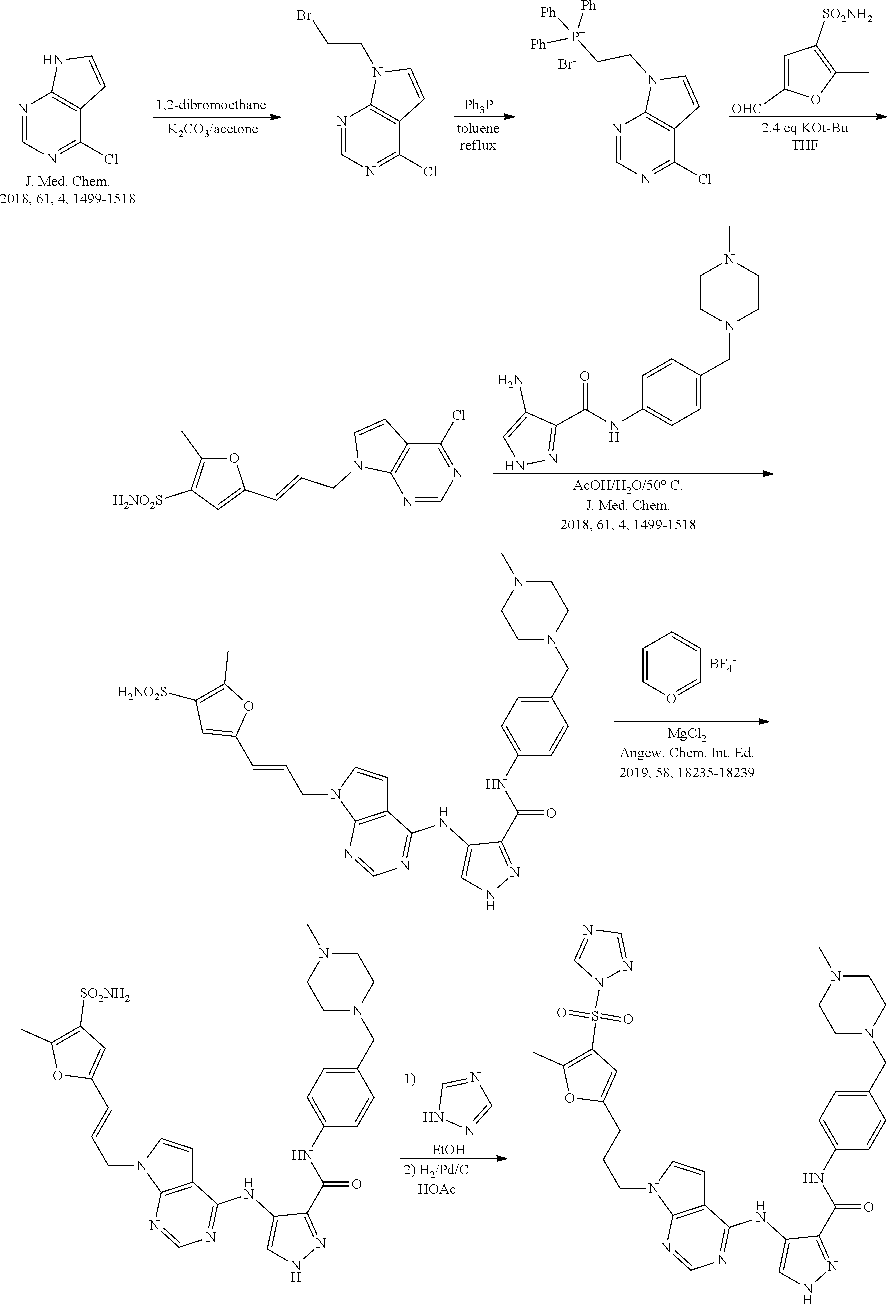

### Synthesis of Compound 176

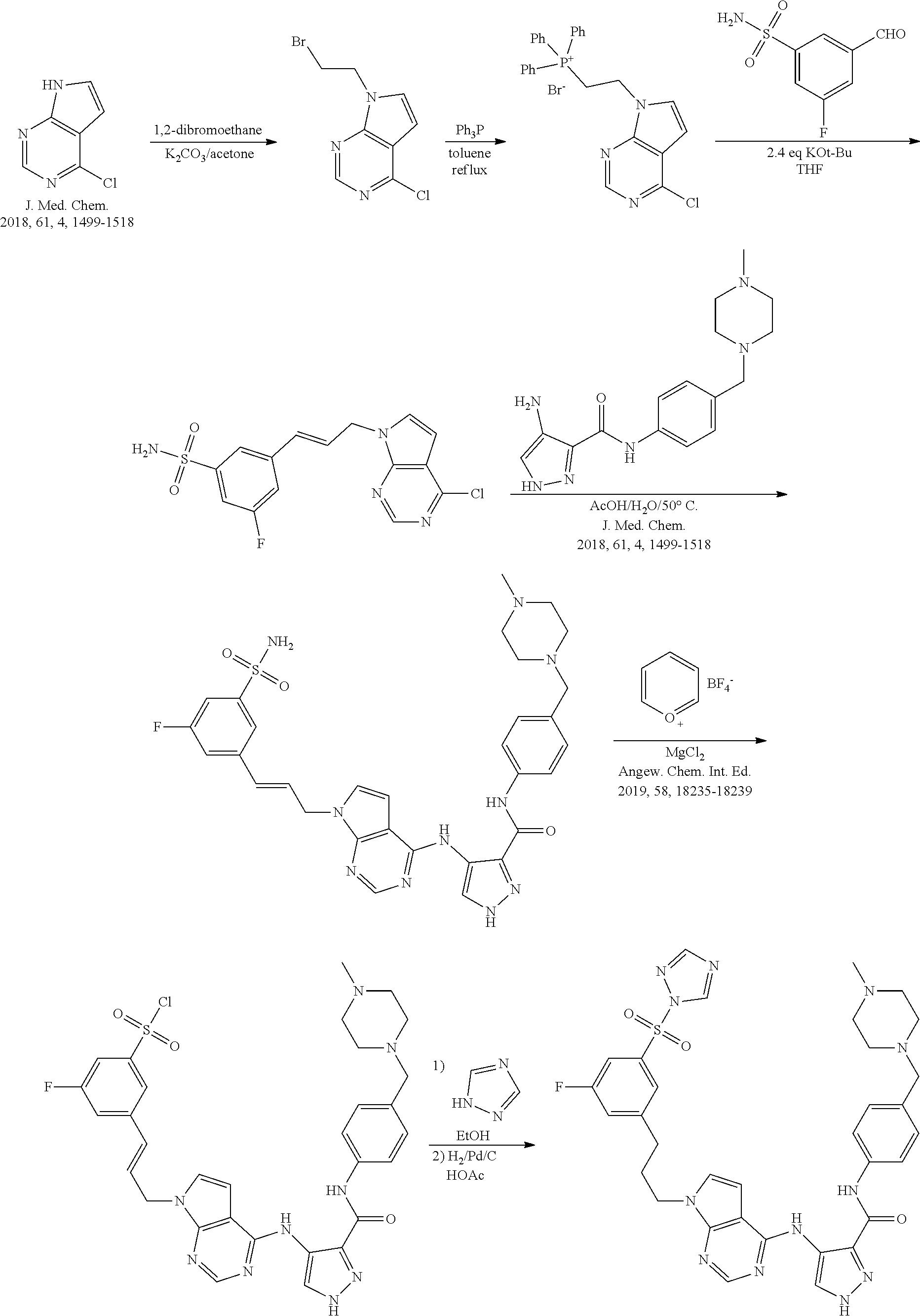

### Synthesis of Compound 177

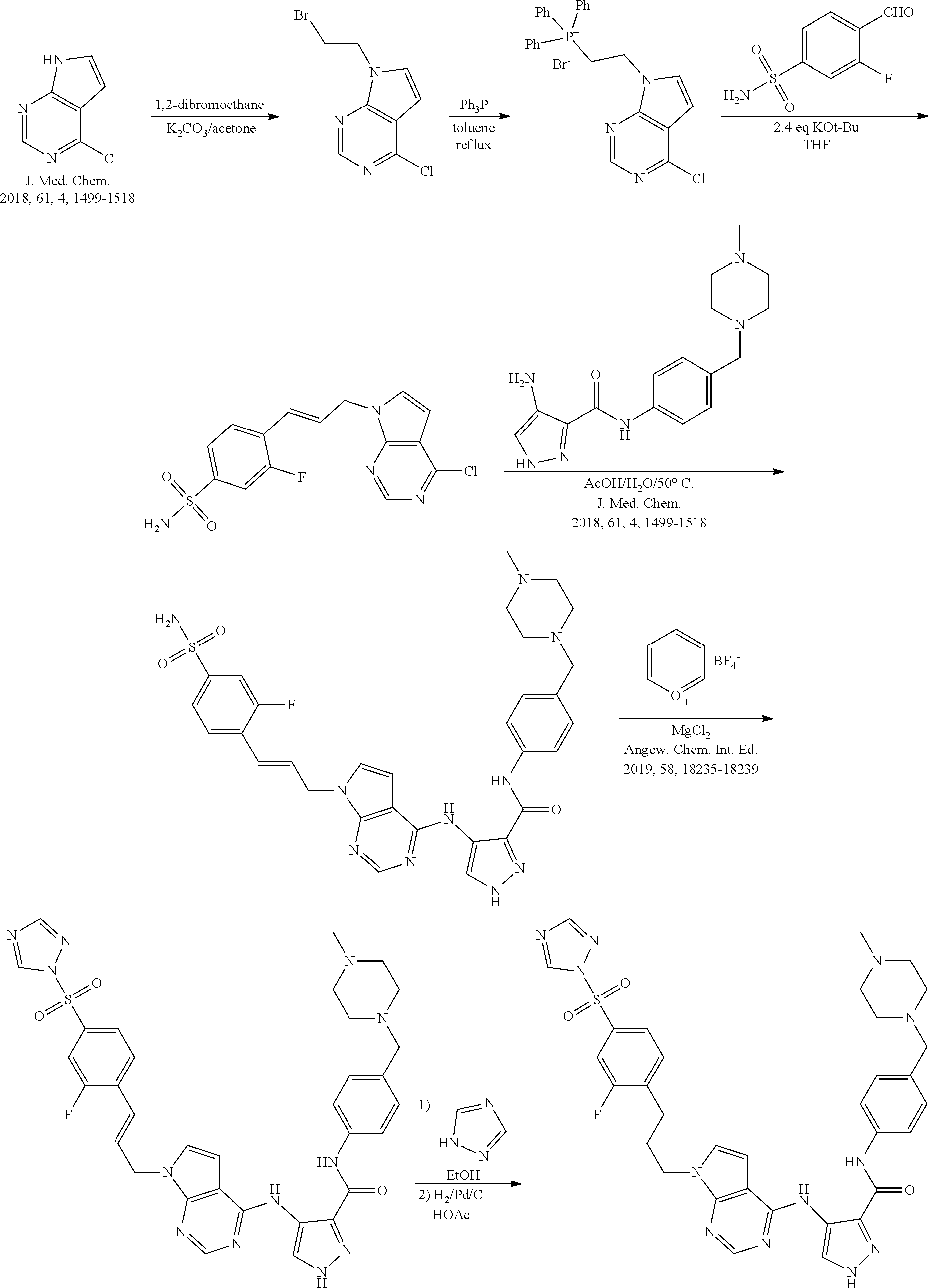

### Synthesis of Compound 178

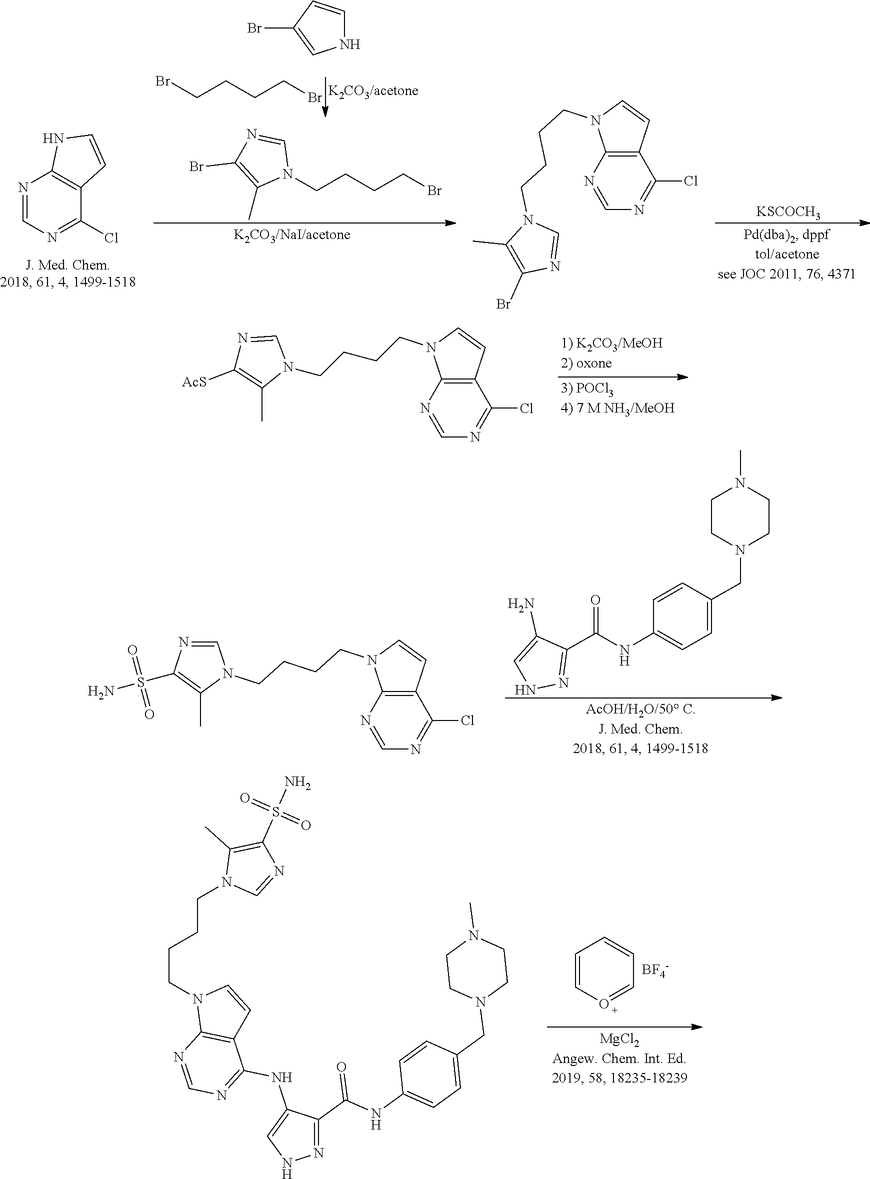

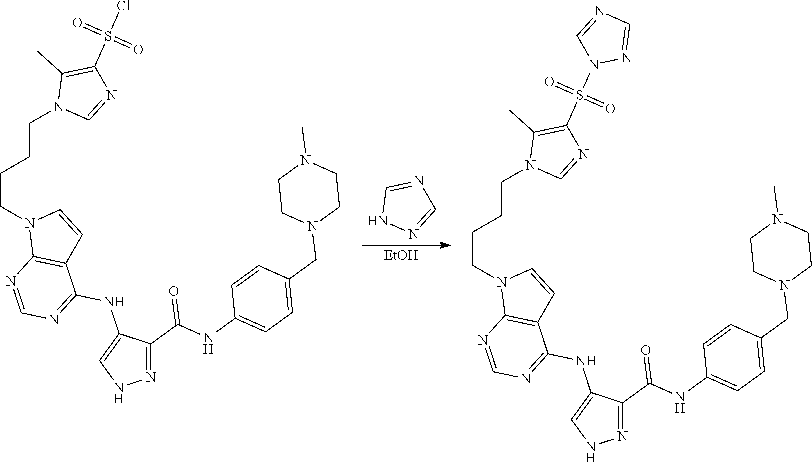

### Synthesis of Compound 179

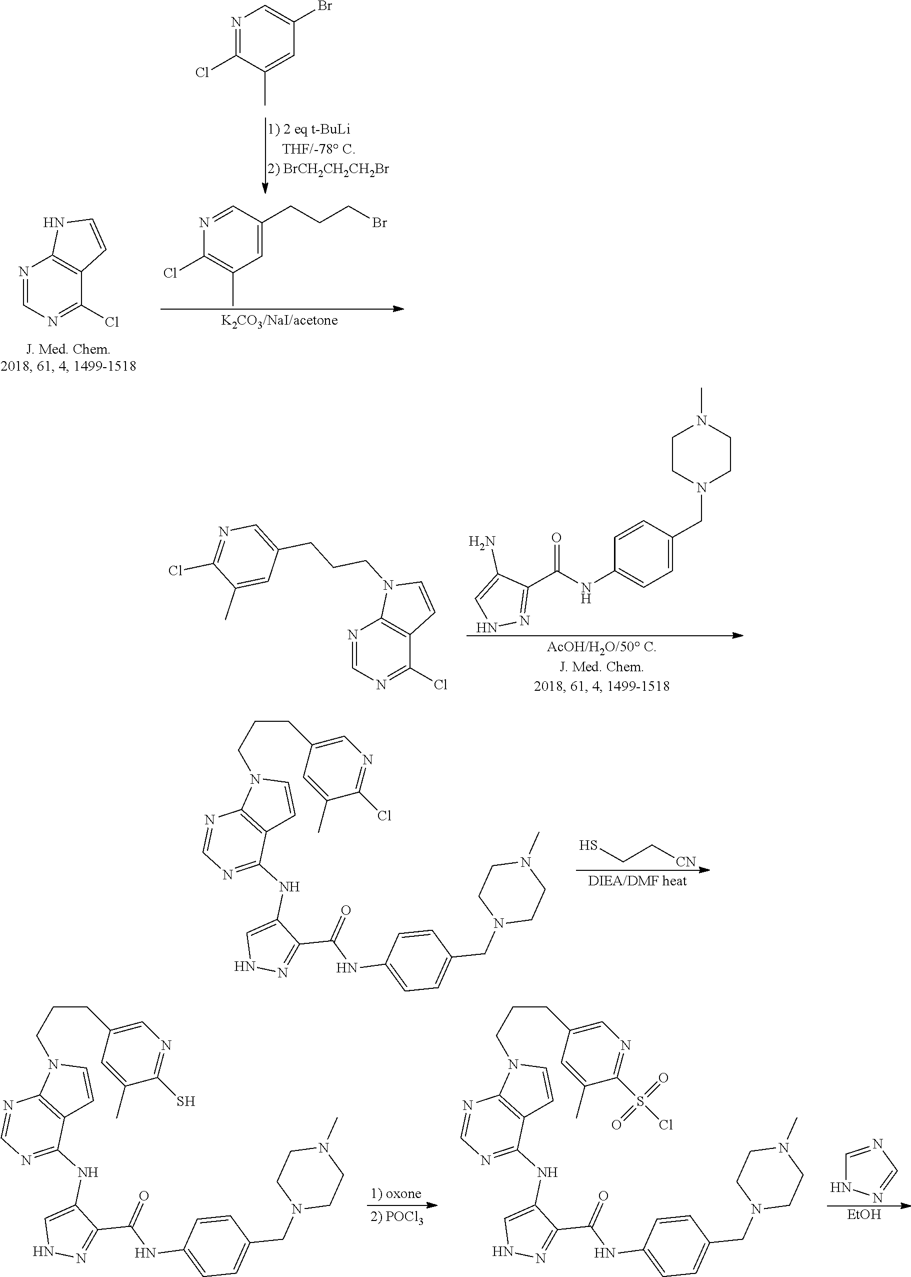

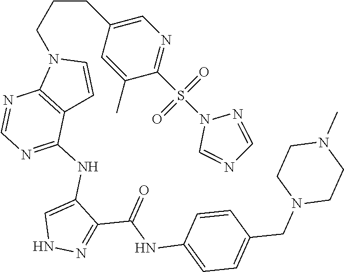 [SMILES: Cc1cc(CCCn2ccc3c(Nc4c[nH]nc4C(=O)Nc4ccc(CN5CCN(C)CC5)cc4)ncnc32)cnc1S(=O)(=O)n1cncn1]

### Synthesis of Compound 180

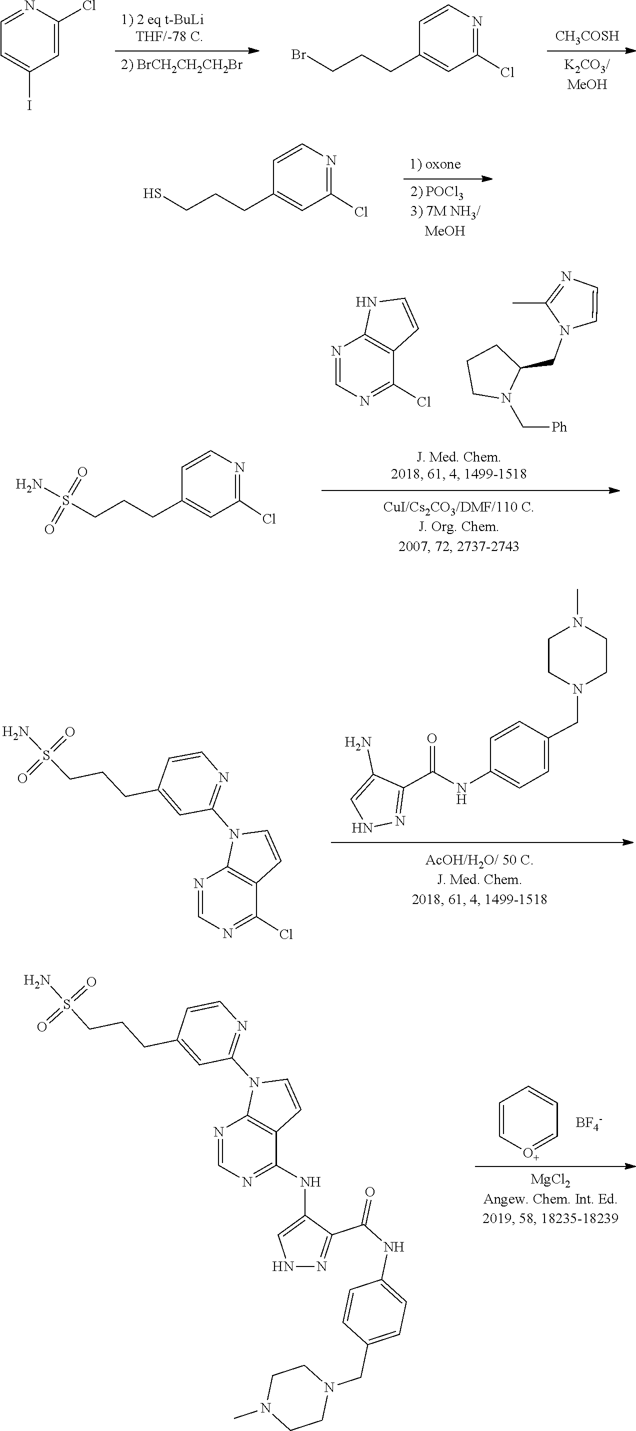

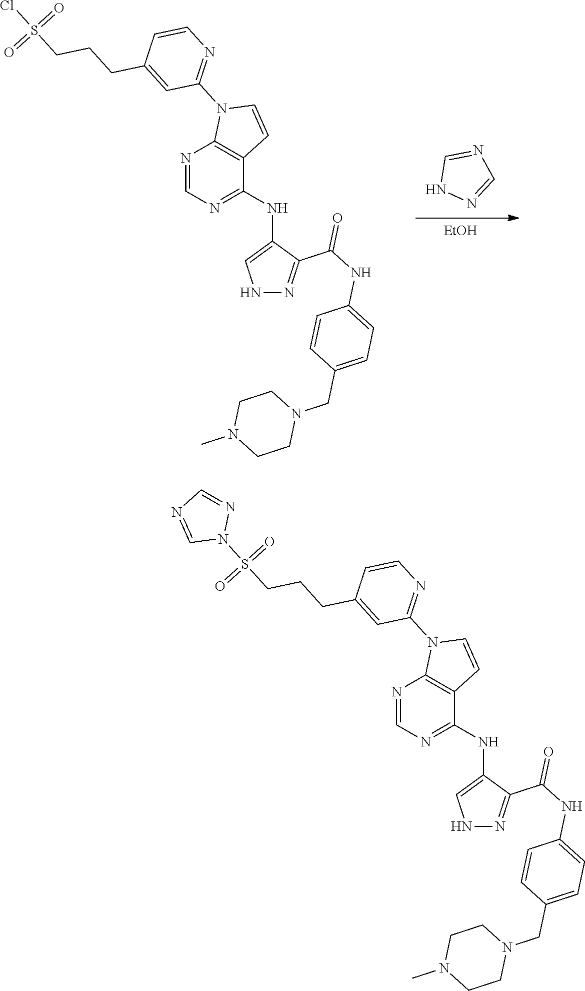

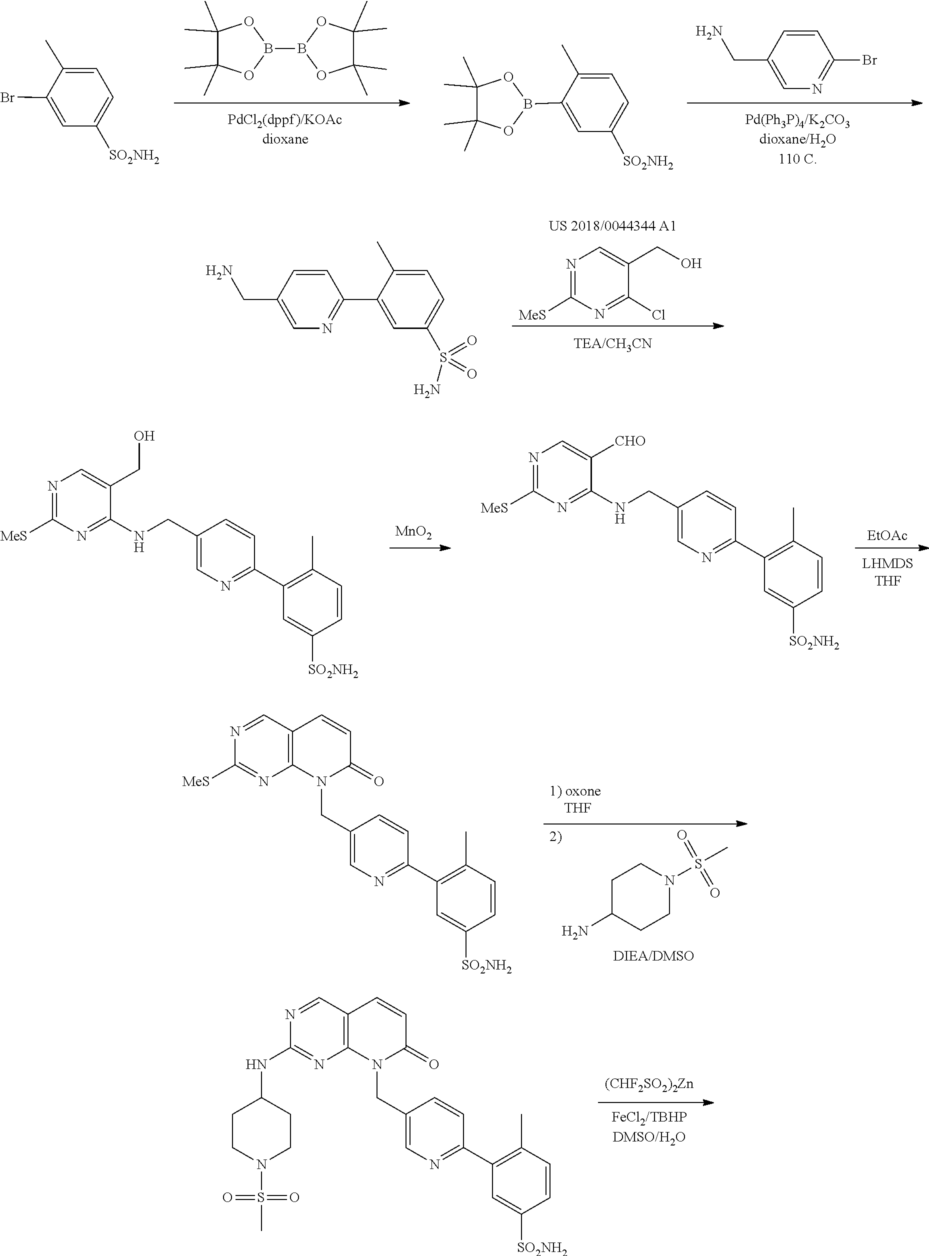

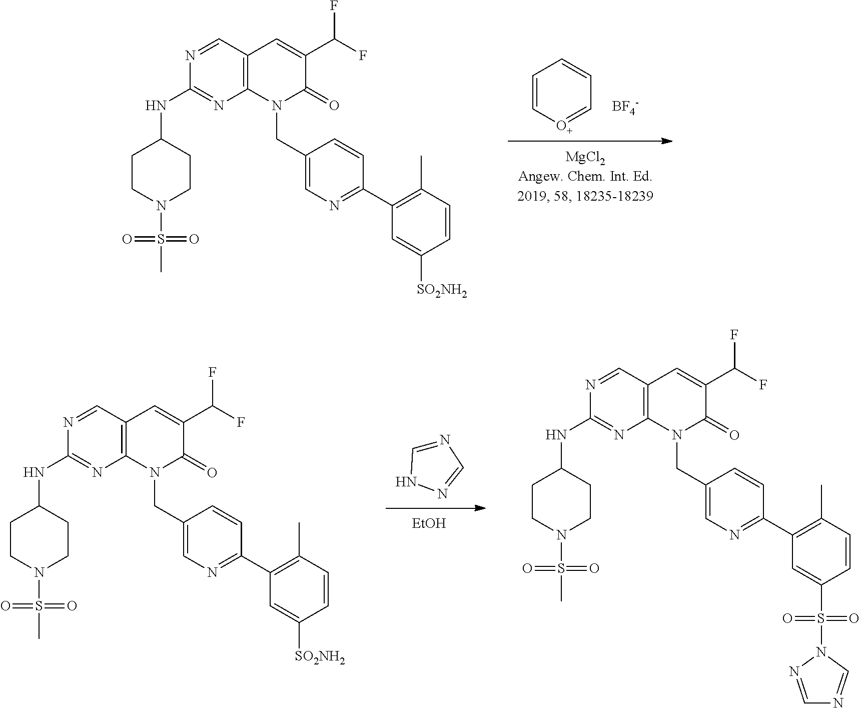

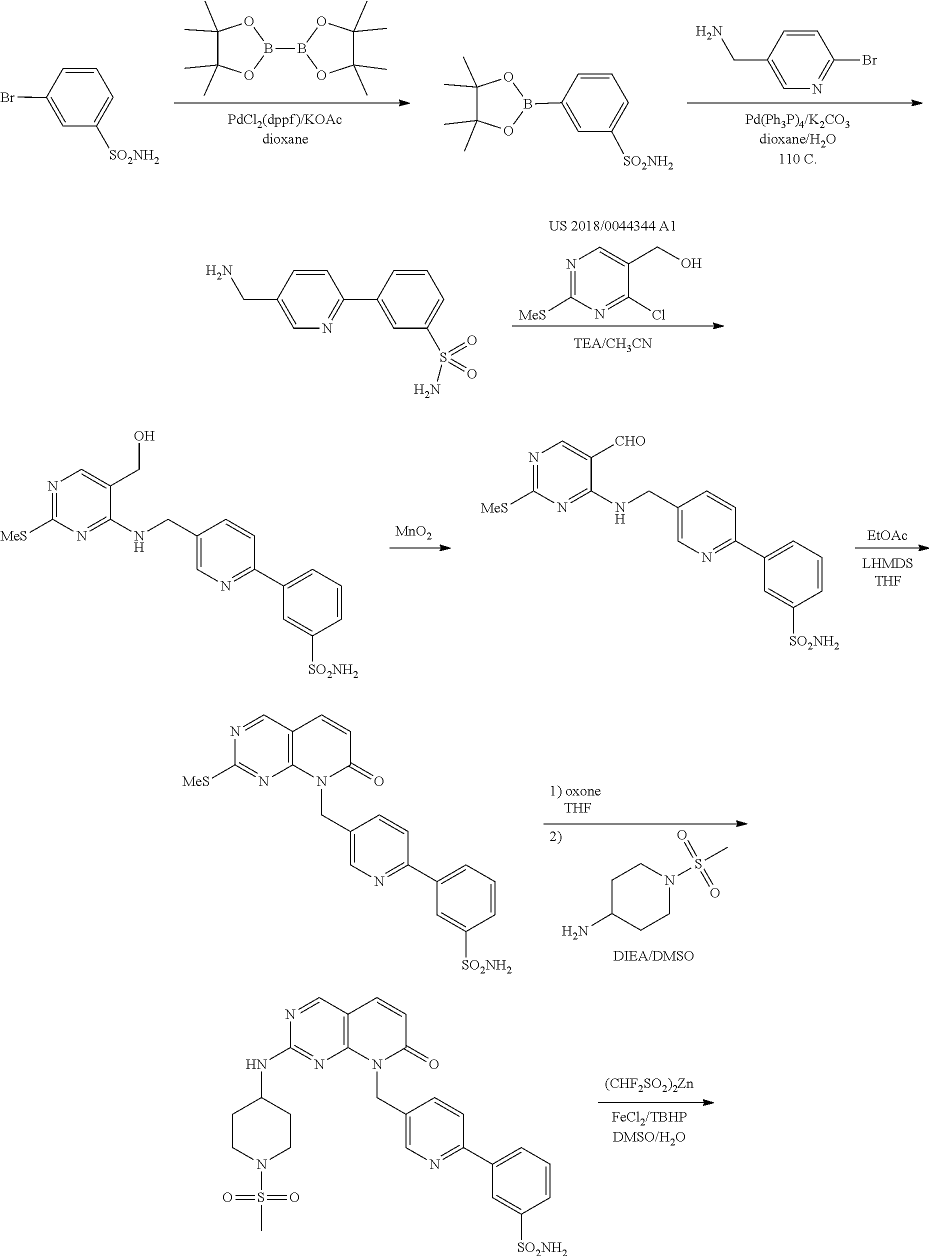

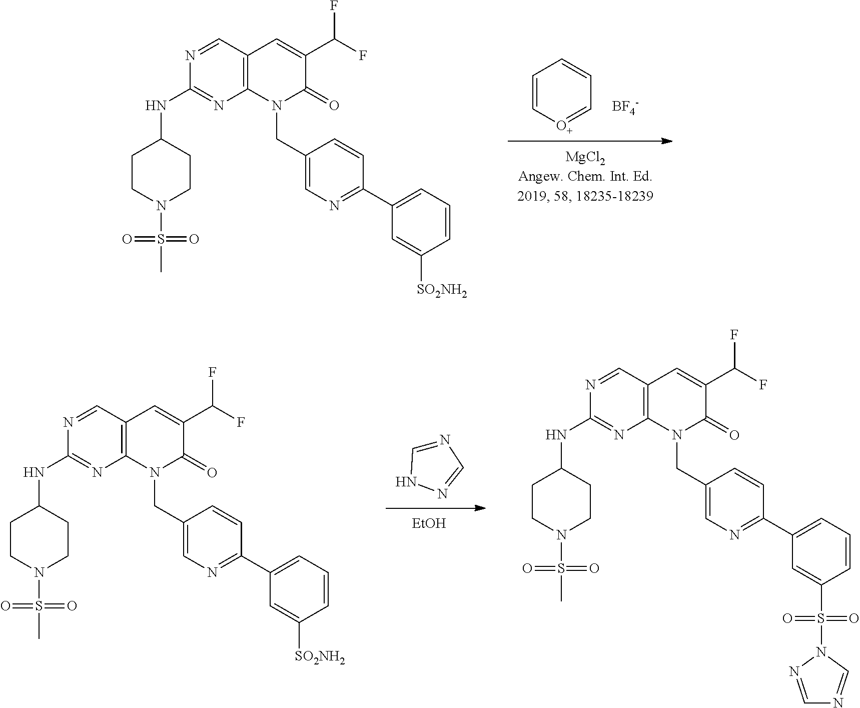

### Synthesis of Compound 183

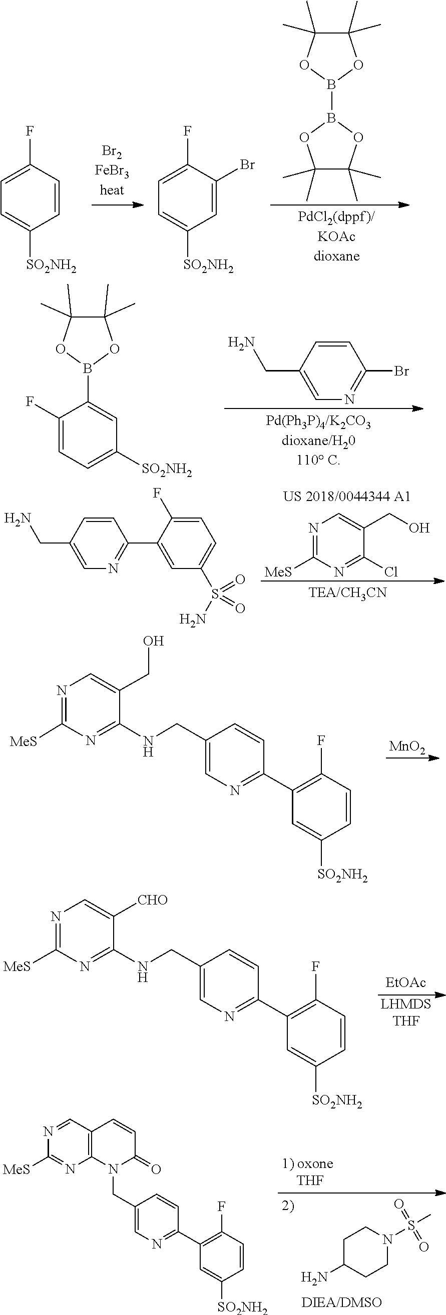

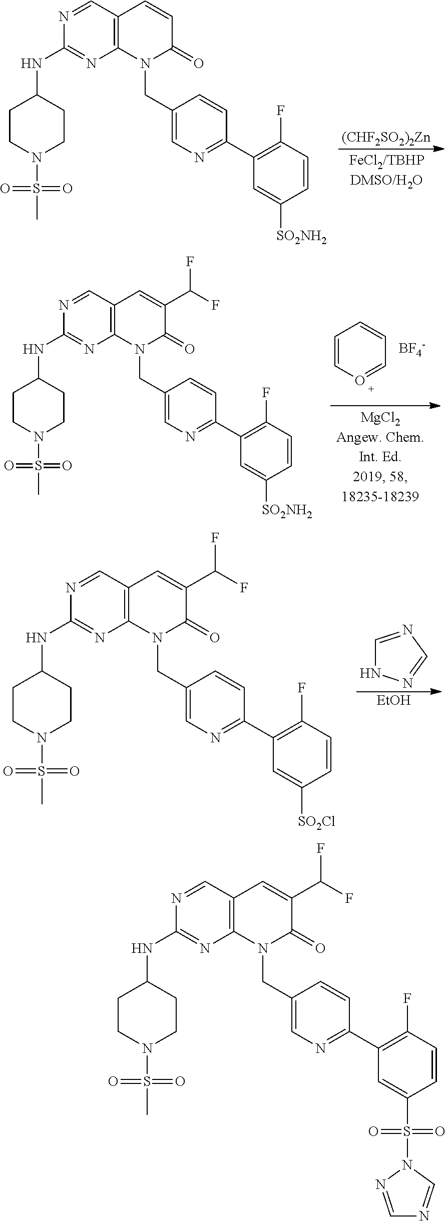

### Synthesis of Compound 184

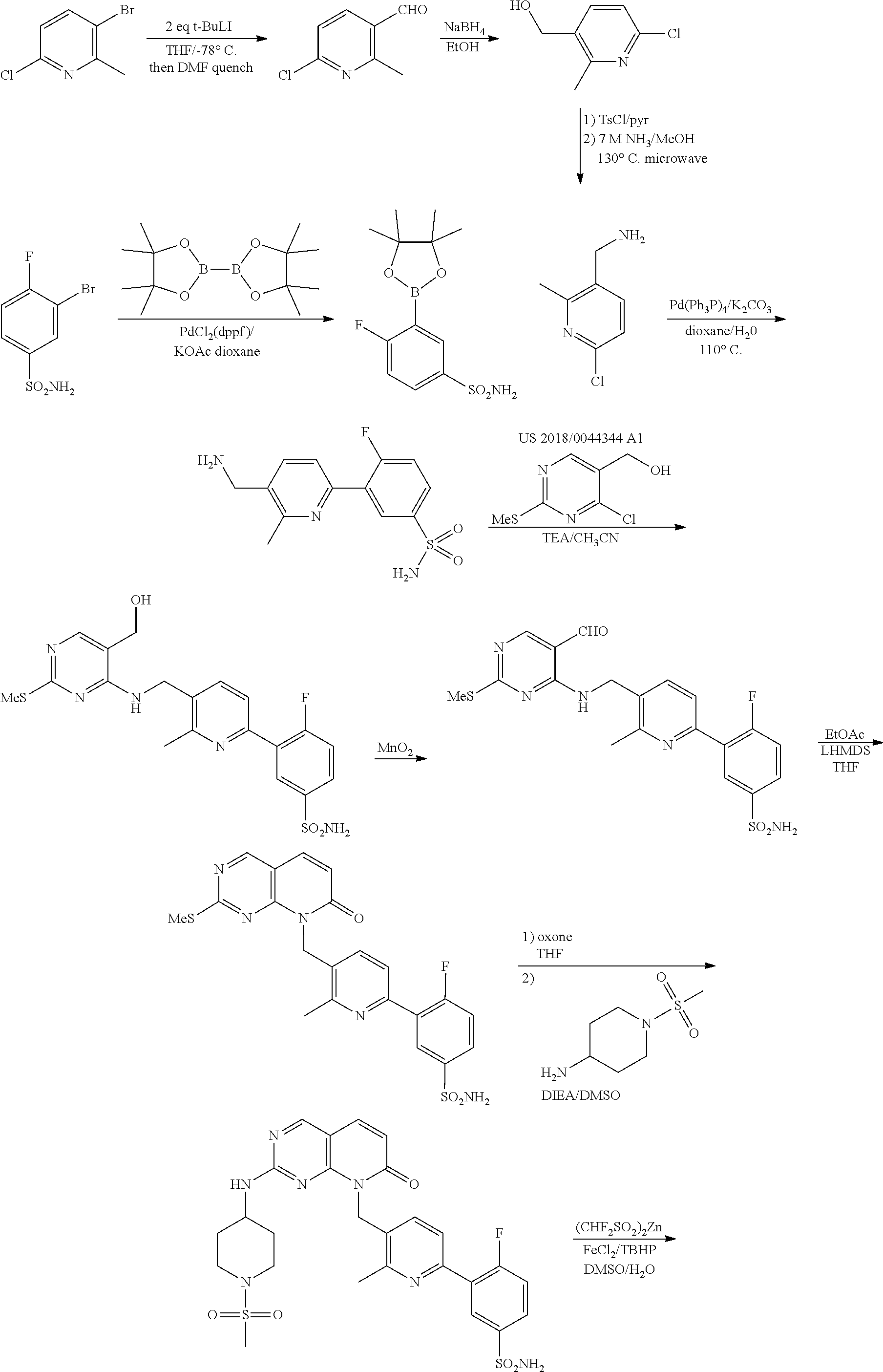

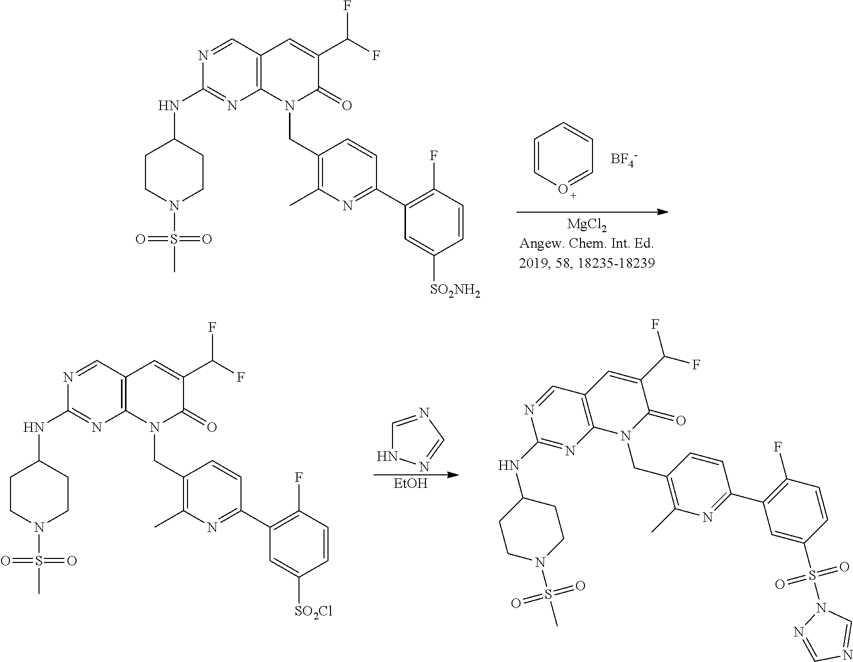

### Synthesis of Compound 185

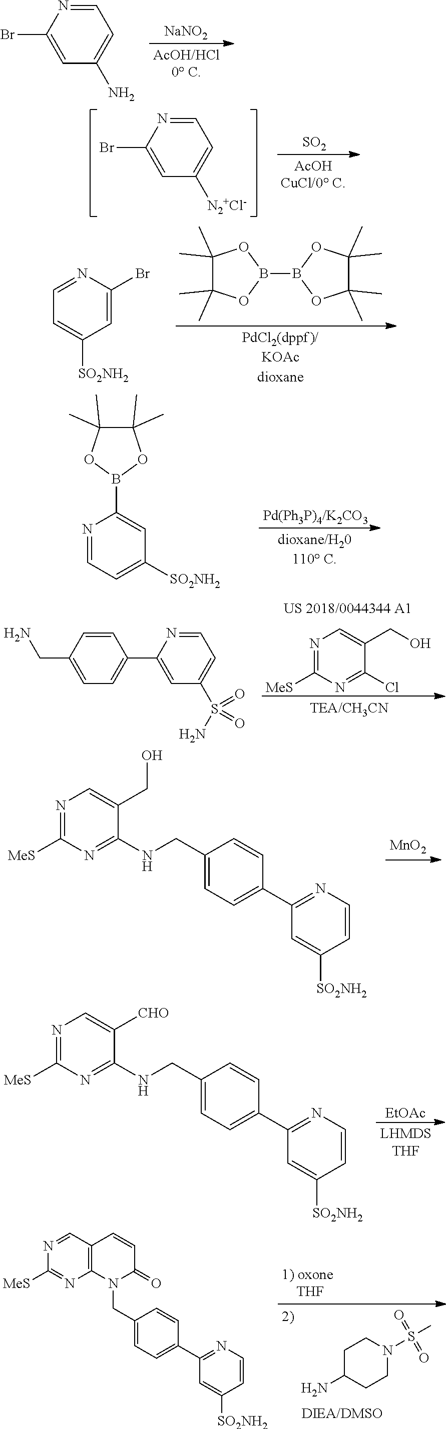

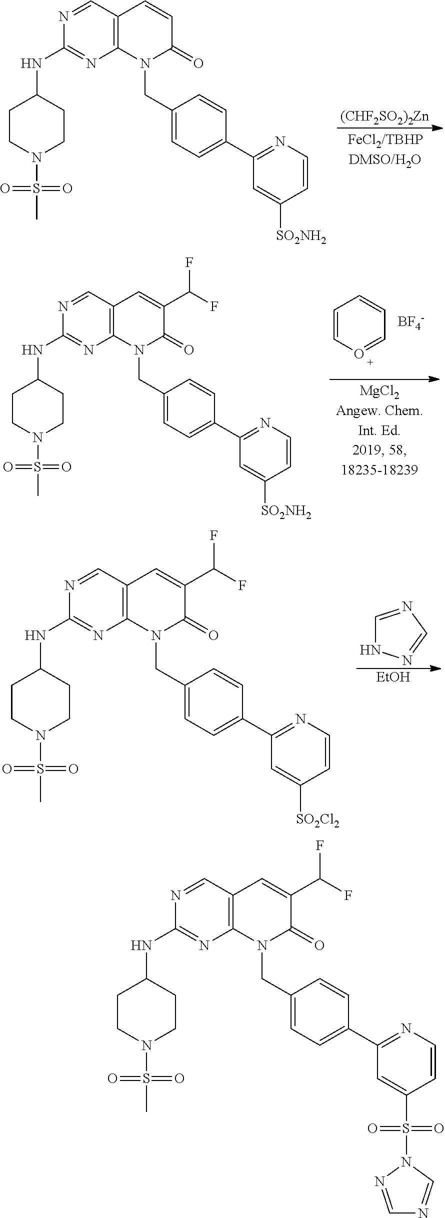

### Synthesis of Compound 186

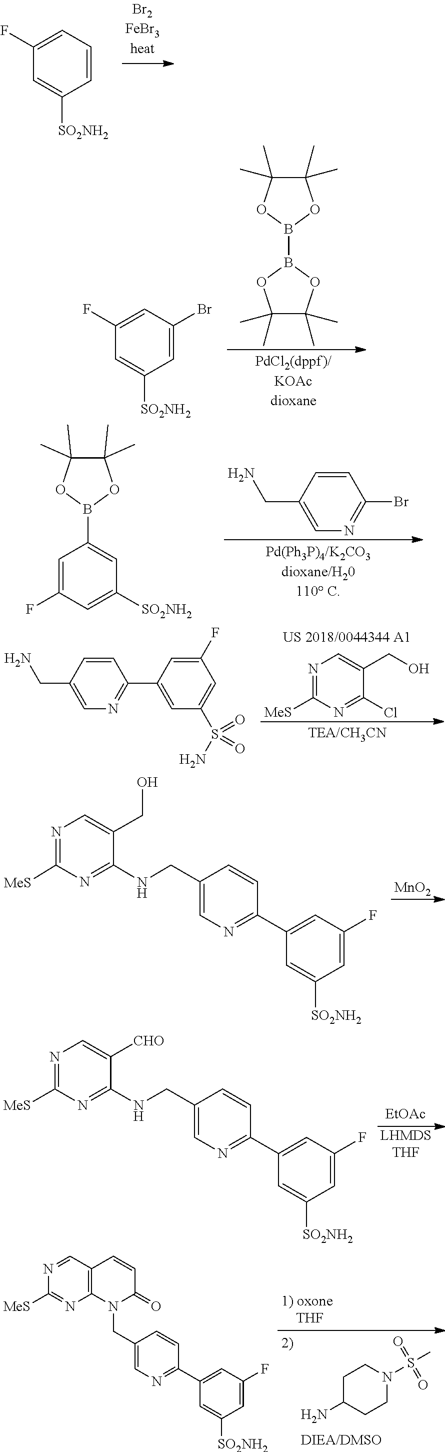

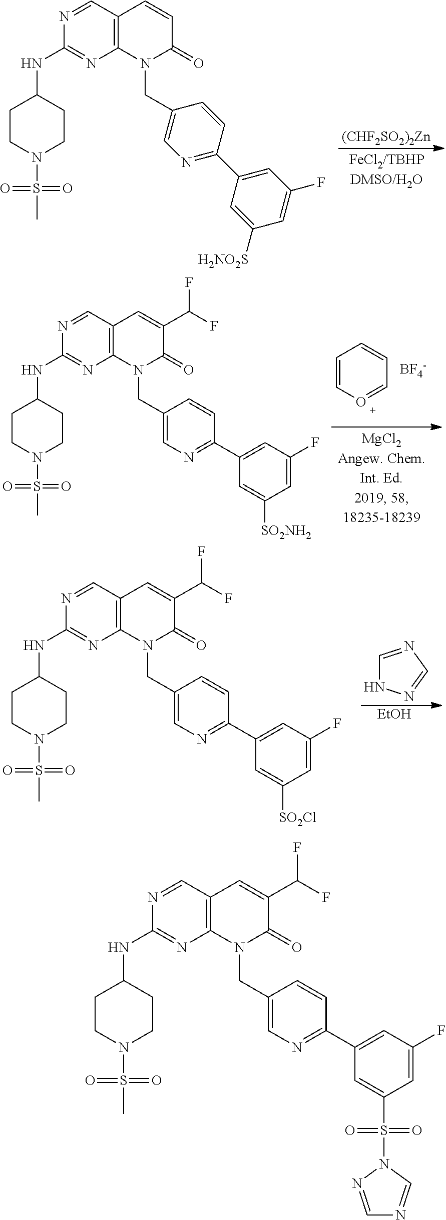

### Synthesis of Compound 187

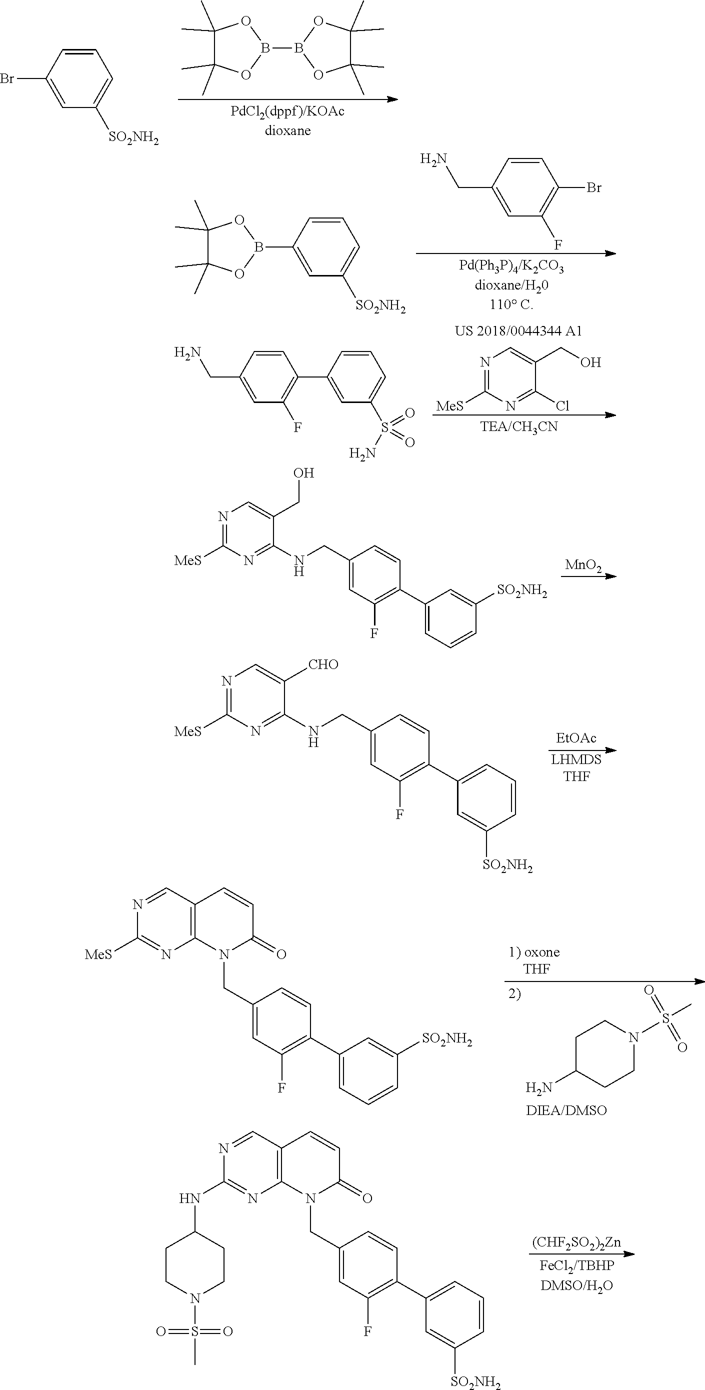

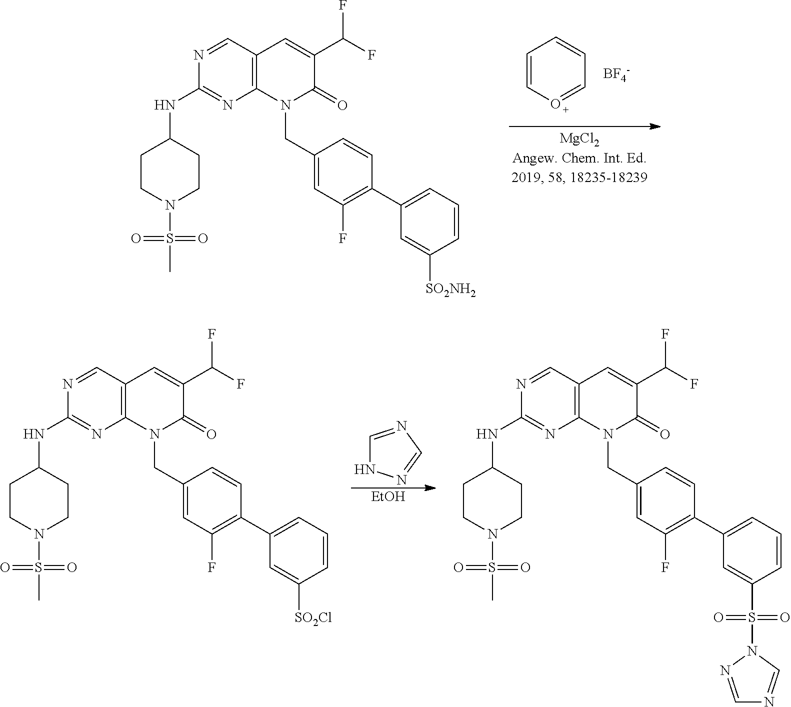

### Synthesis of Compound 188

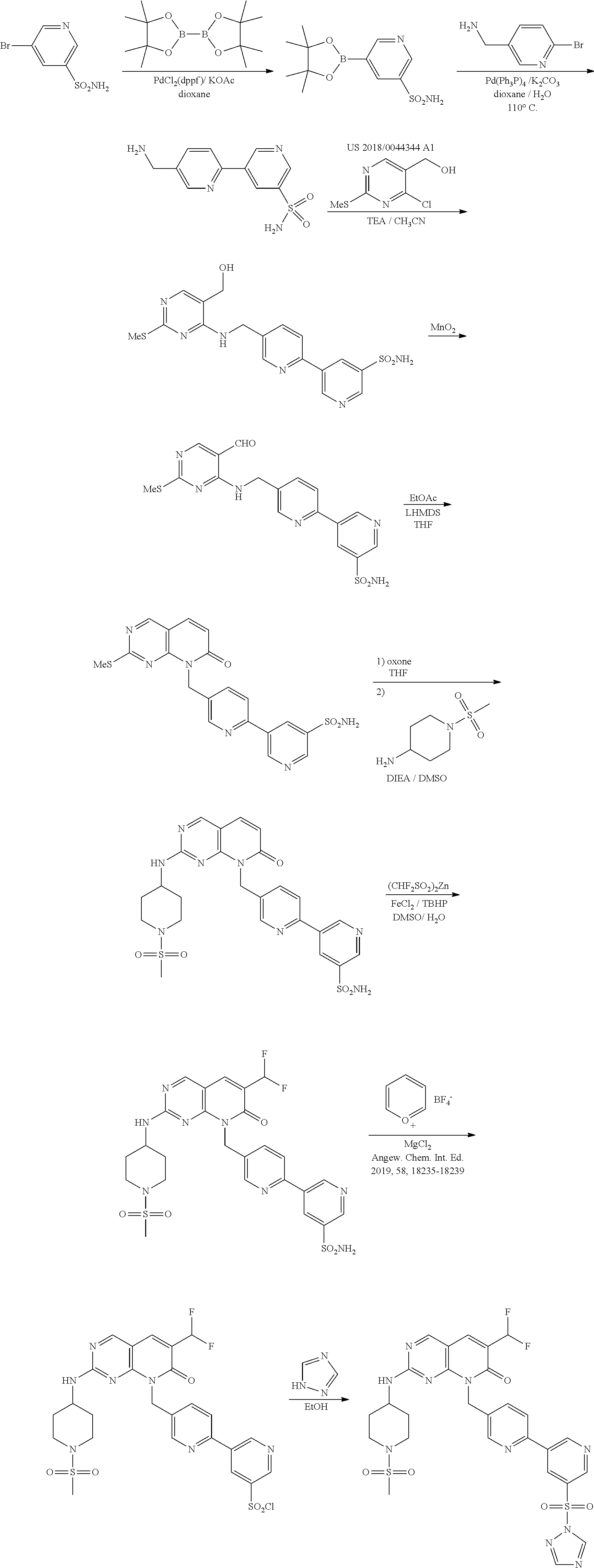

### Synthesis of Compound 189

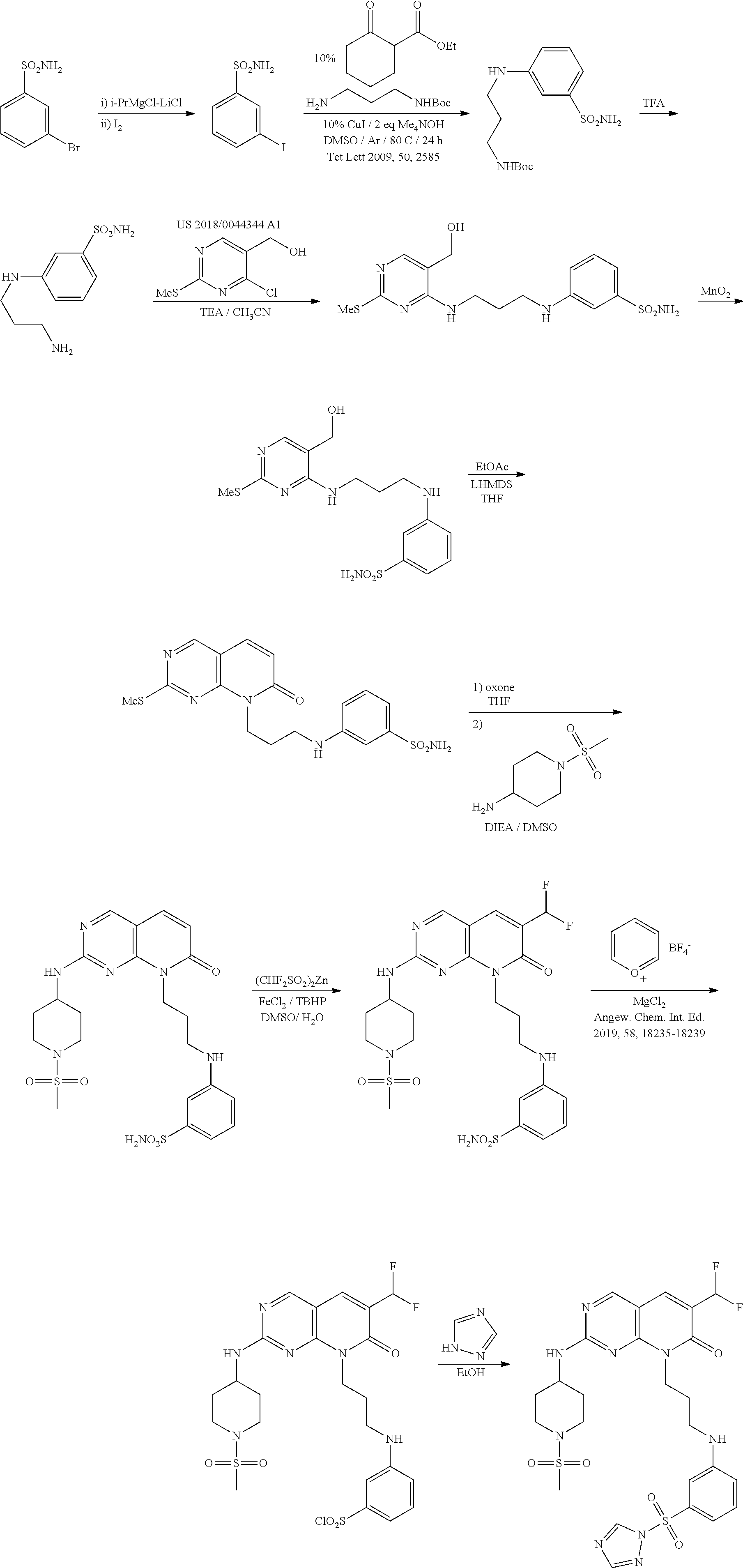

### Synthesis of Compound 190

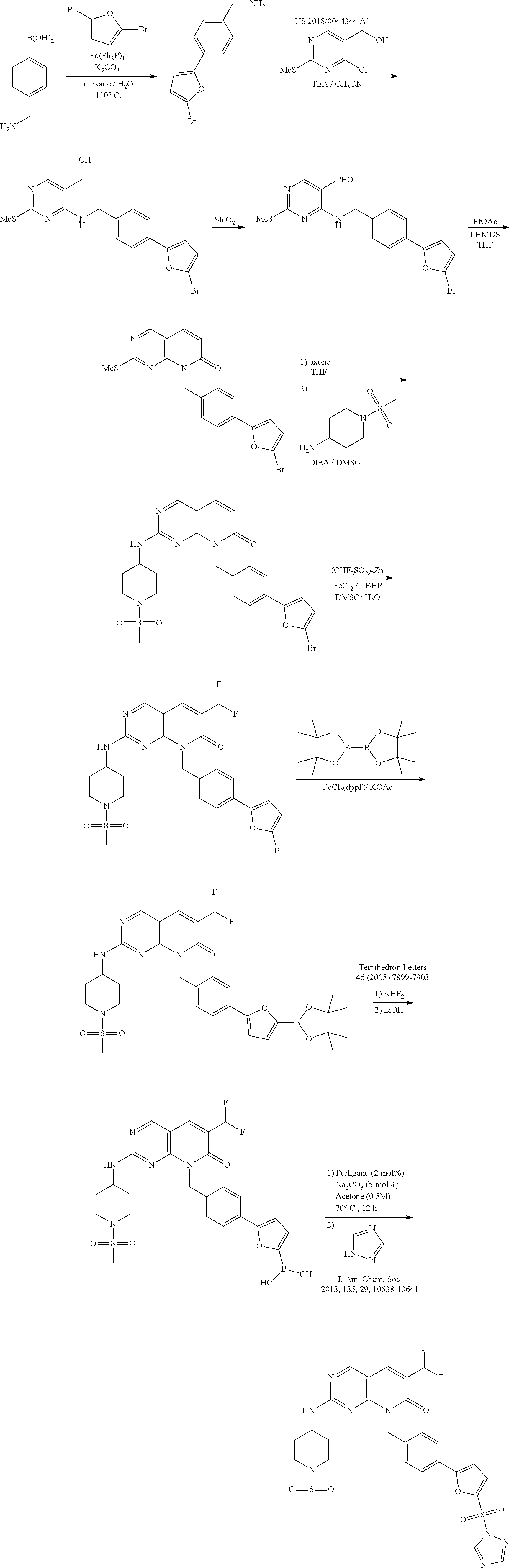

### Synthesis of Compound 191

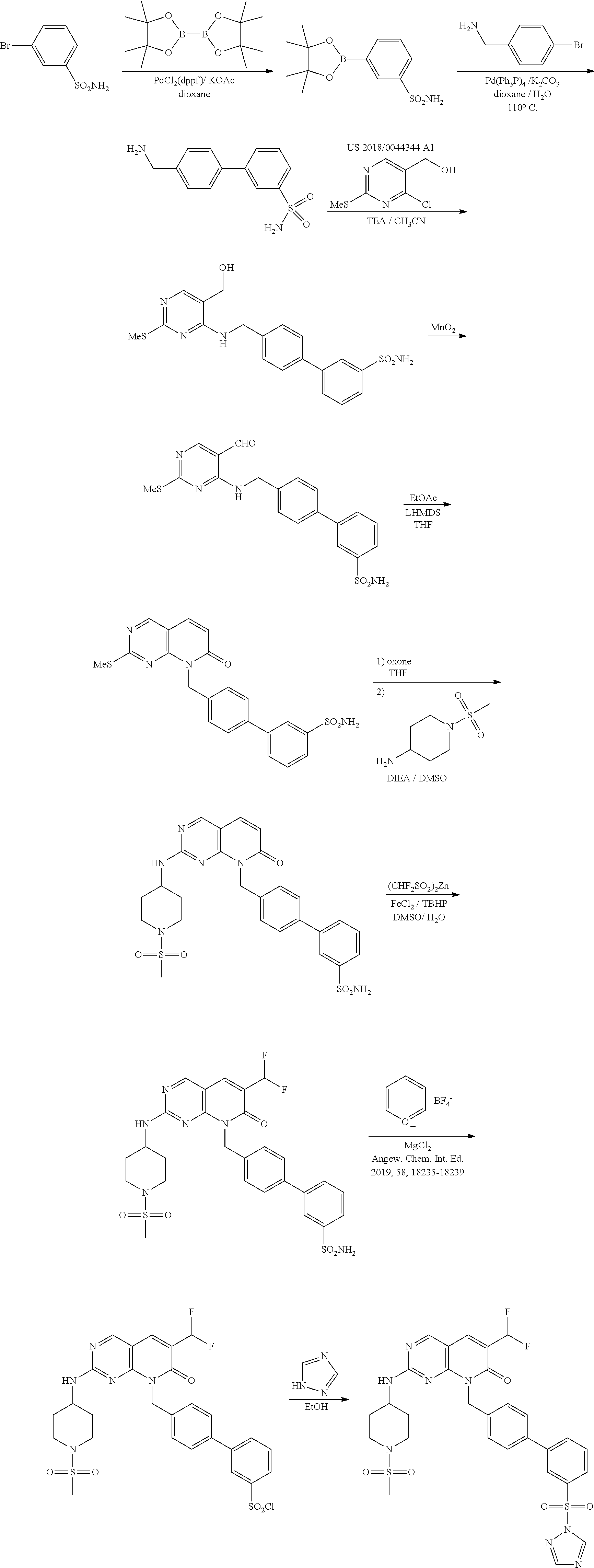

### Synthesis of Compound 192

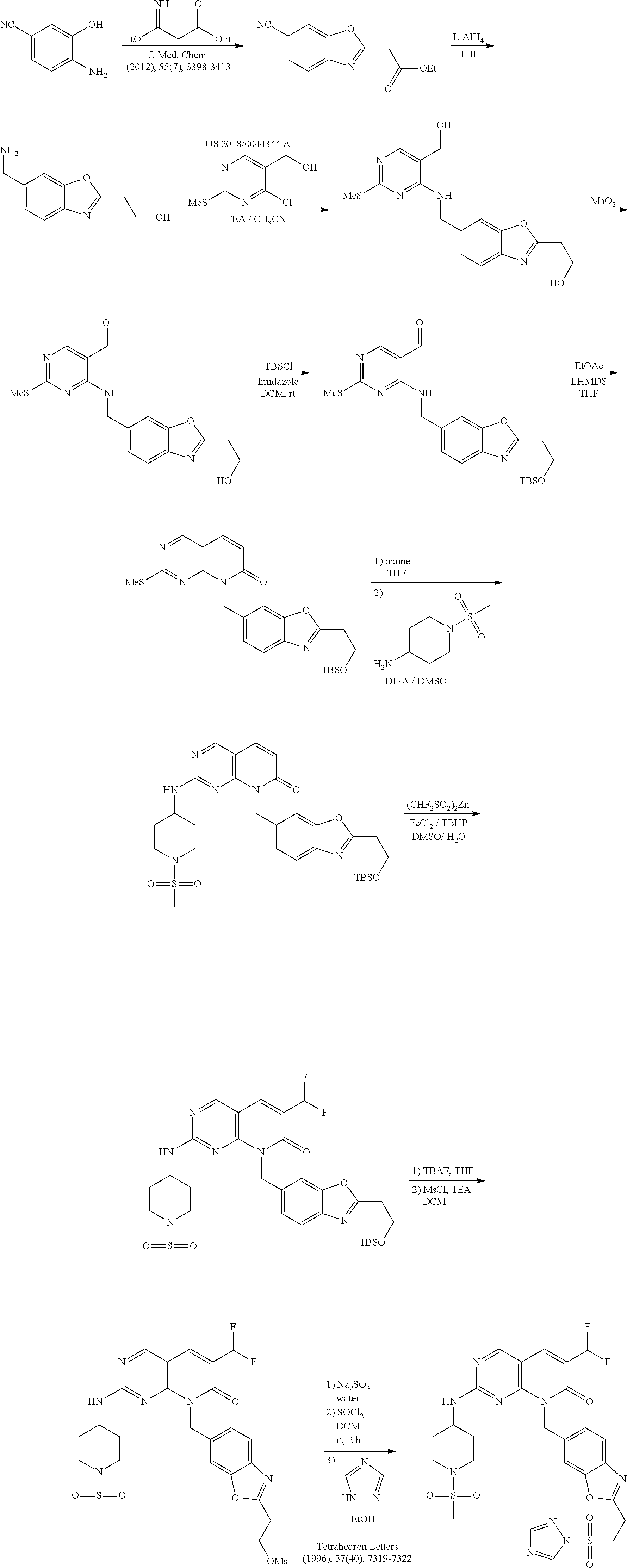

### Synthesis of Compound 193

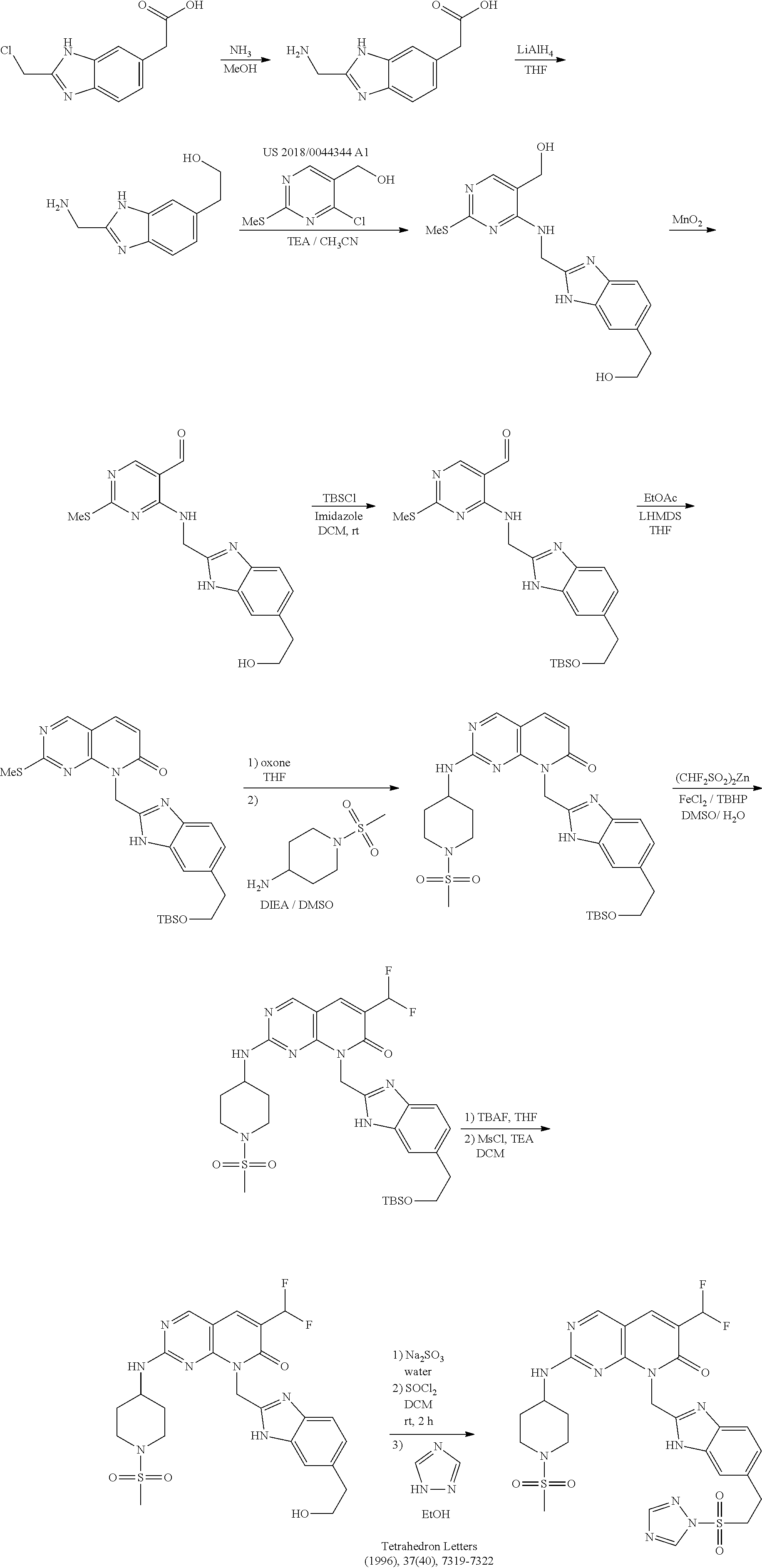

### Synthesis of Compound 194

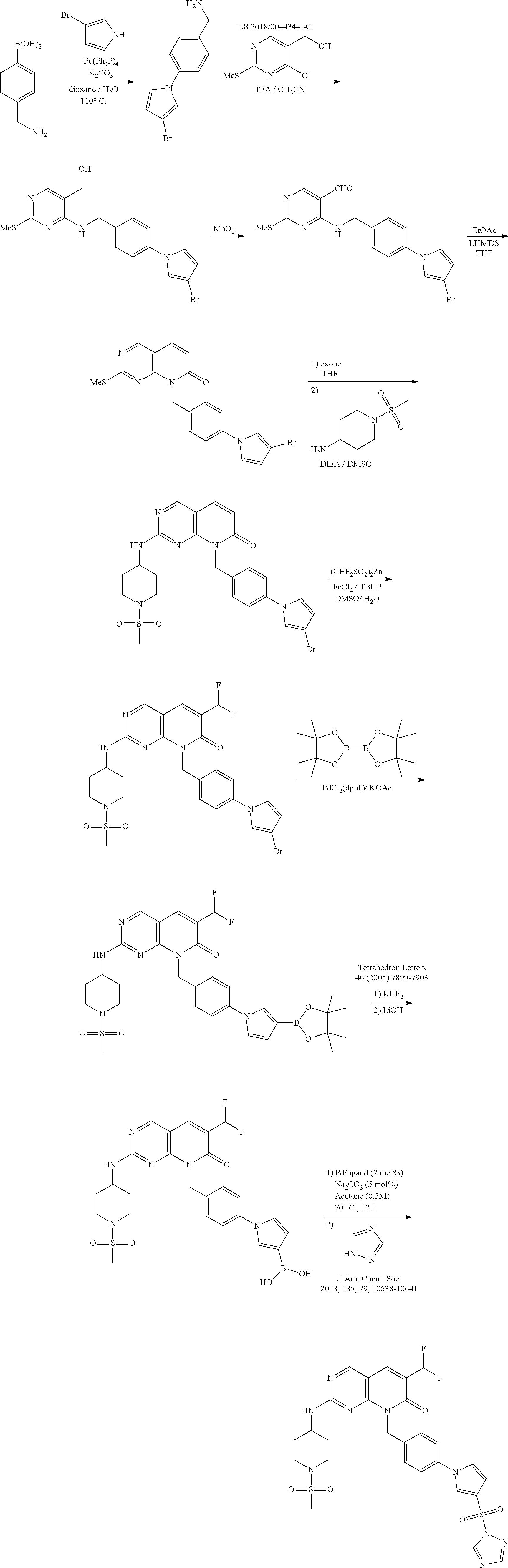

### Synthesis of Compound 195

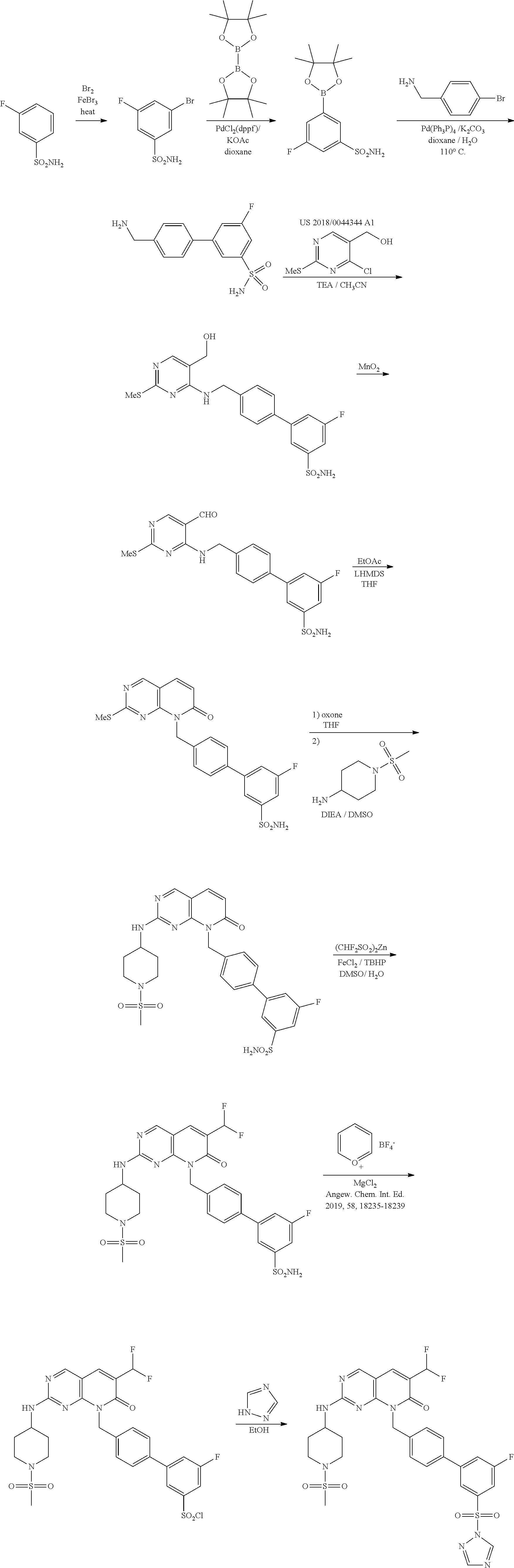

### Synthesis of Compound 196

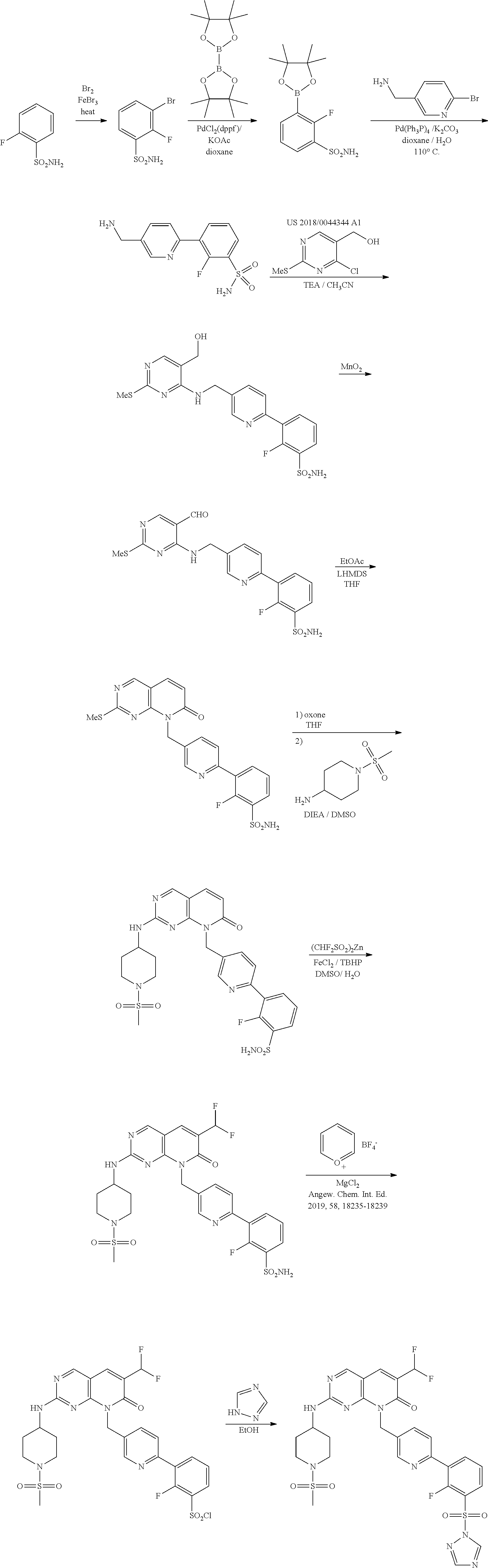

### Synthesis of Compound 197

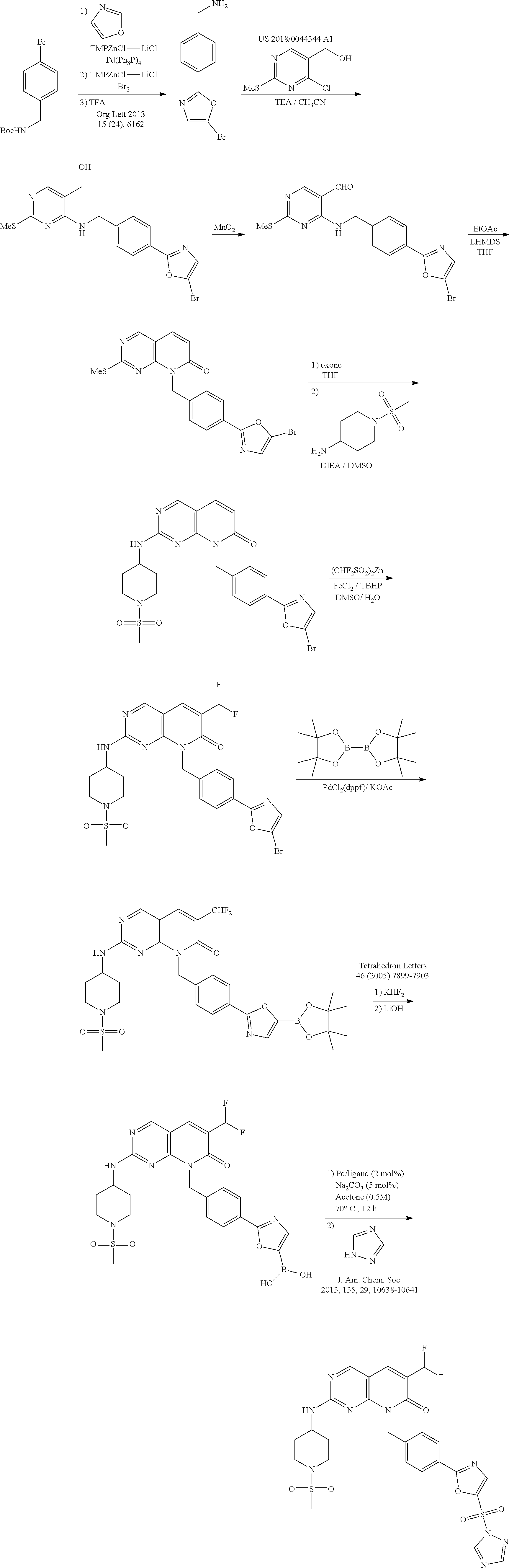

### Synthesis of Compound 198

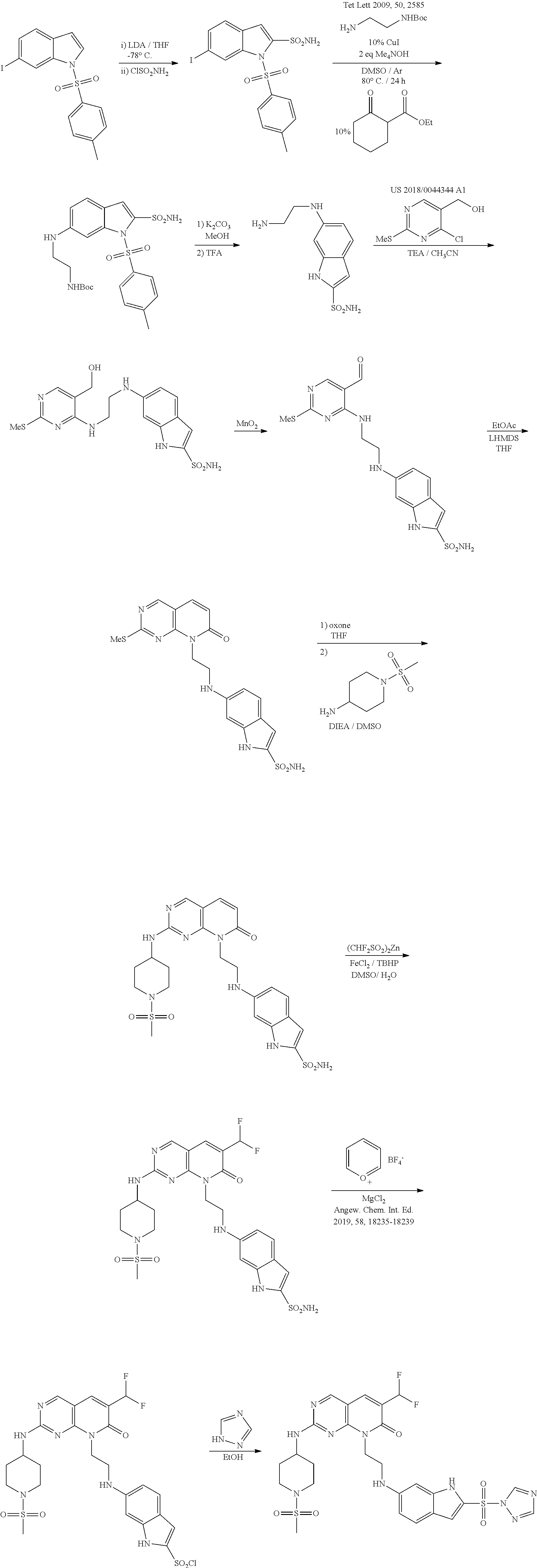

### Synthesis of Compound 199

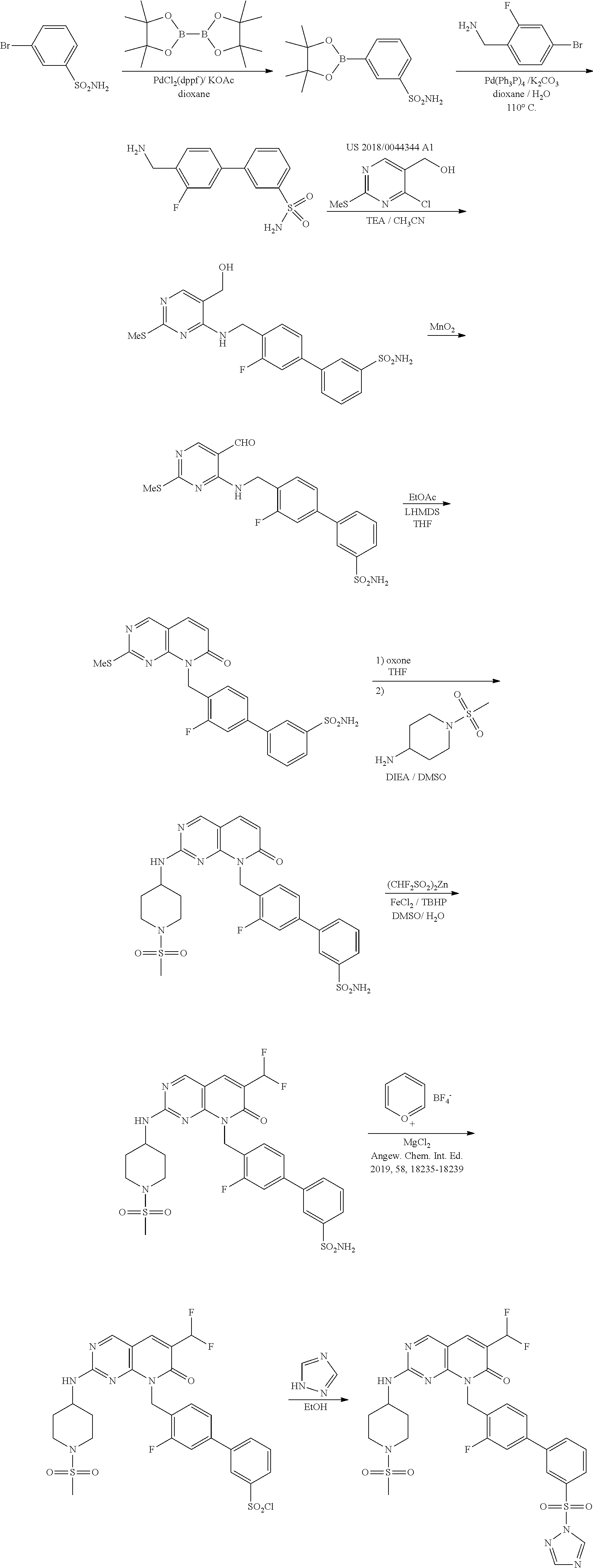

### Synthesis of Compound 200

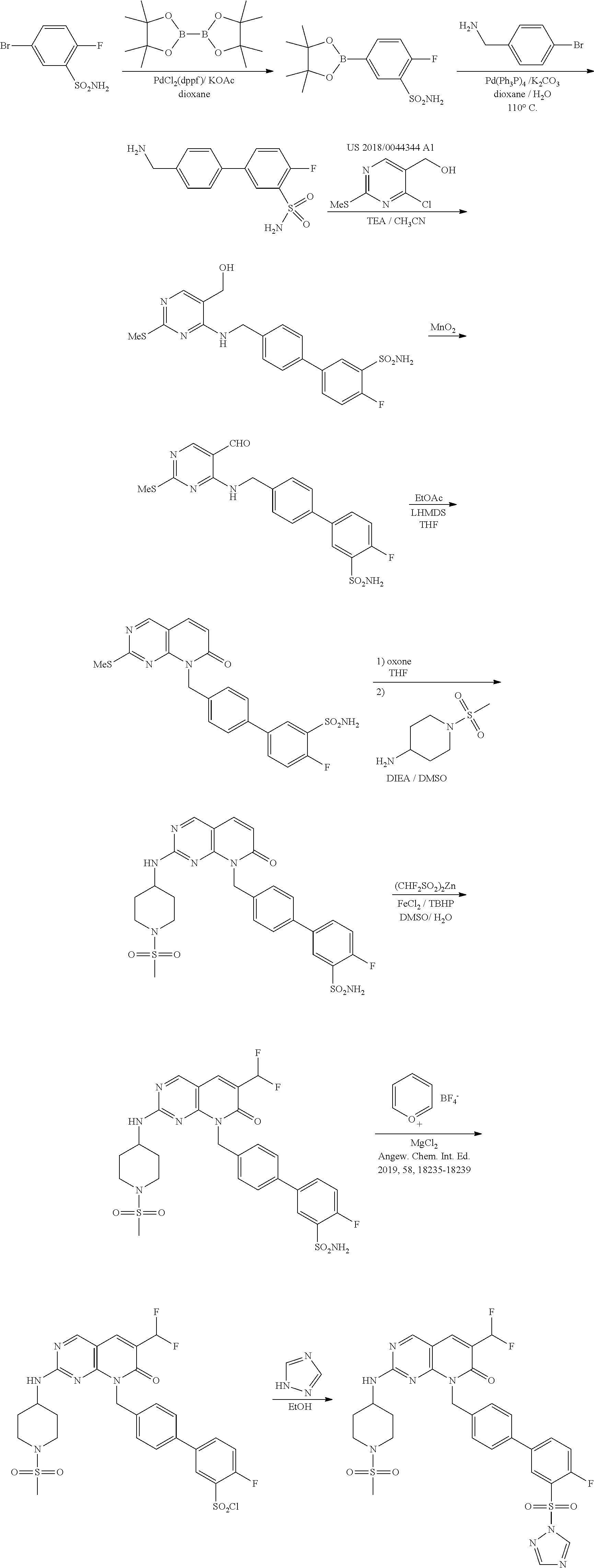

### Synthesis of Compound 201

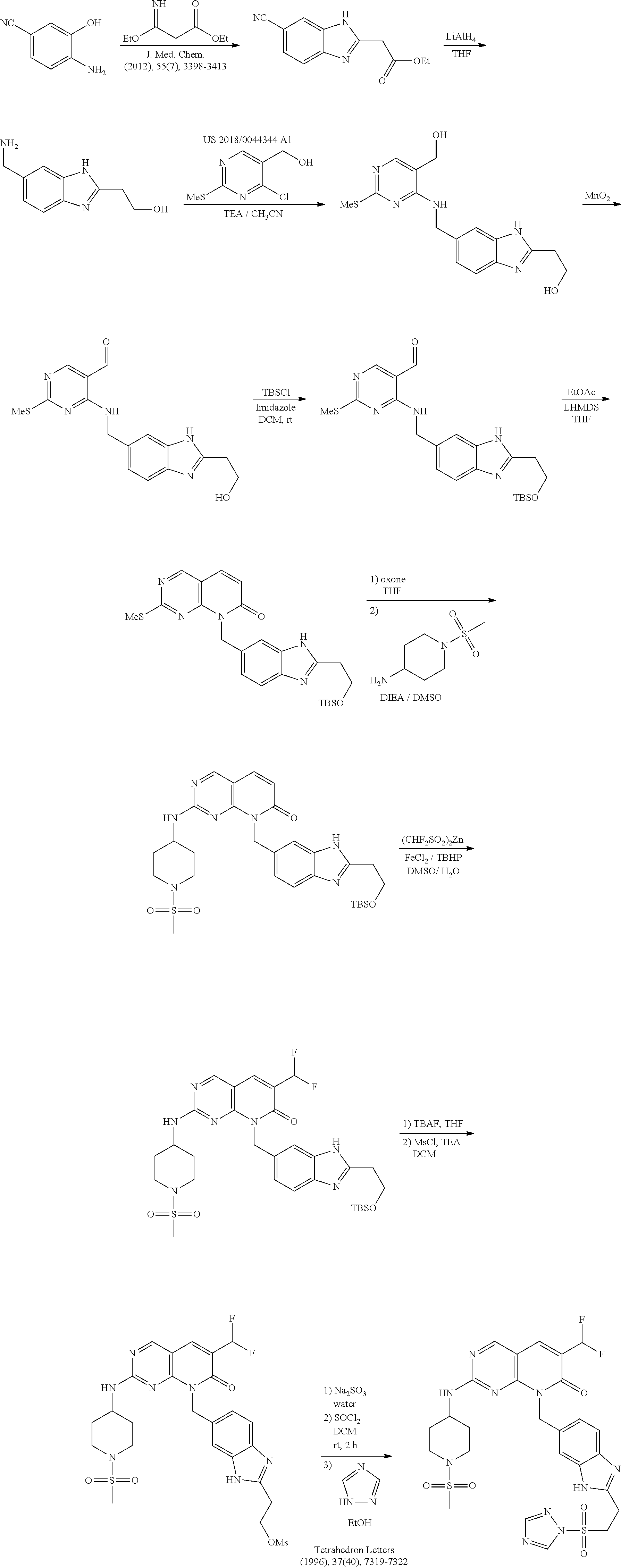

### Synthesis of Compound 202

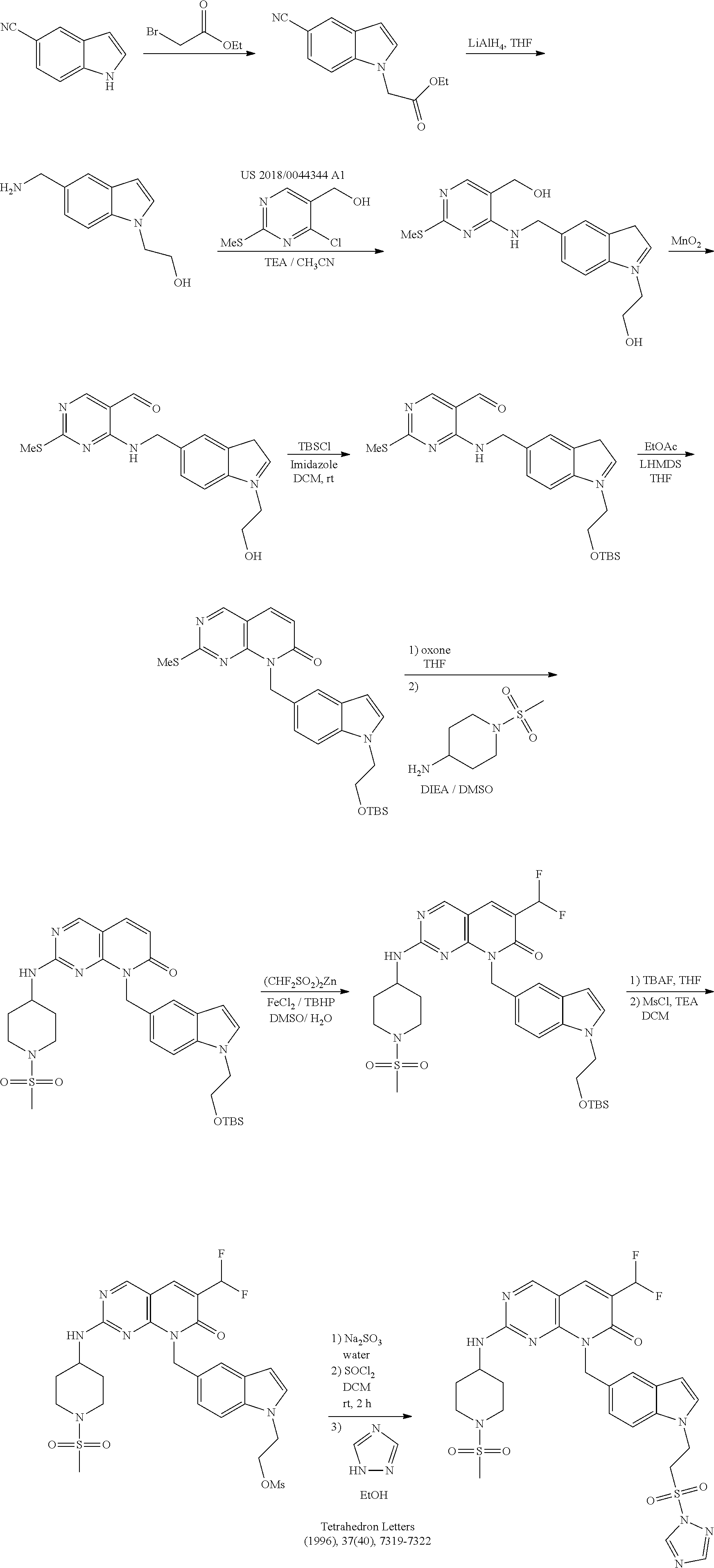

### Synthesis of Compound 203

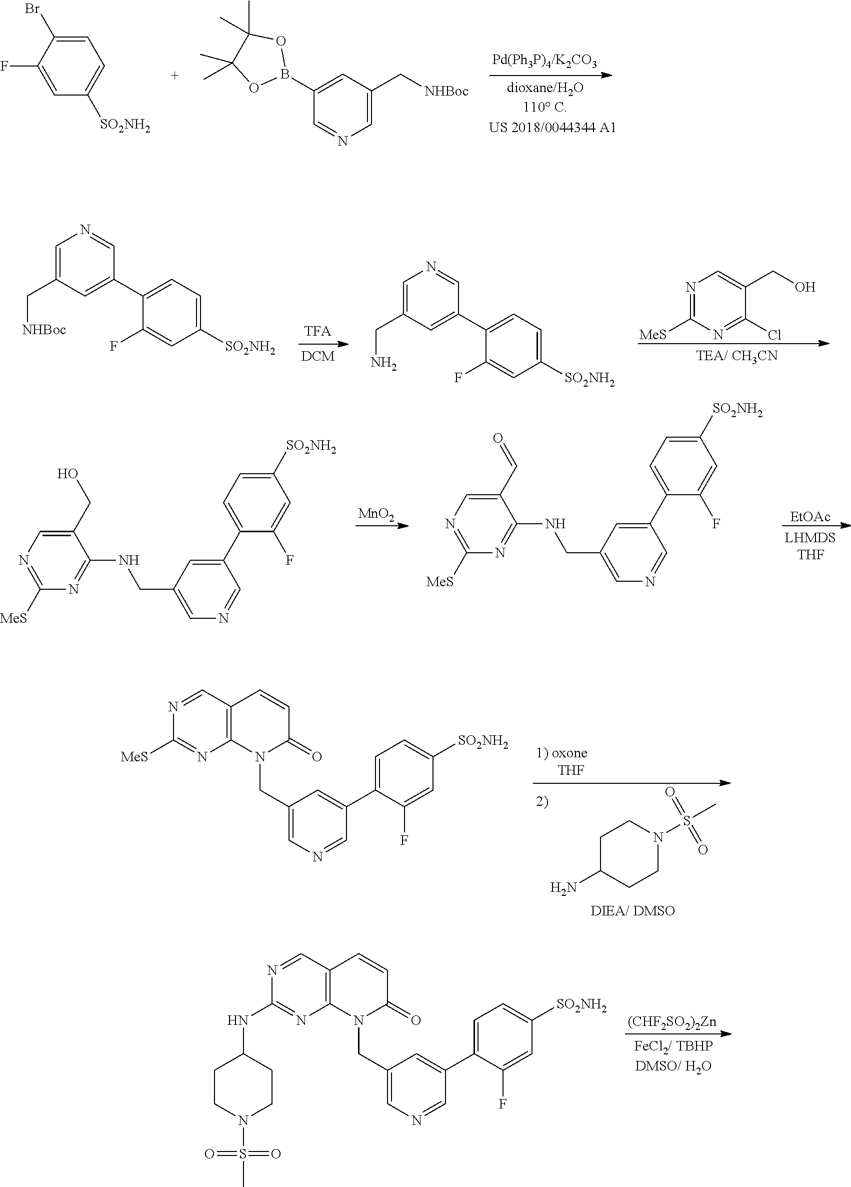

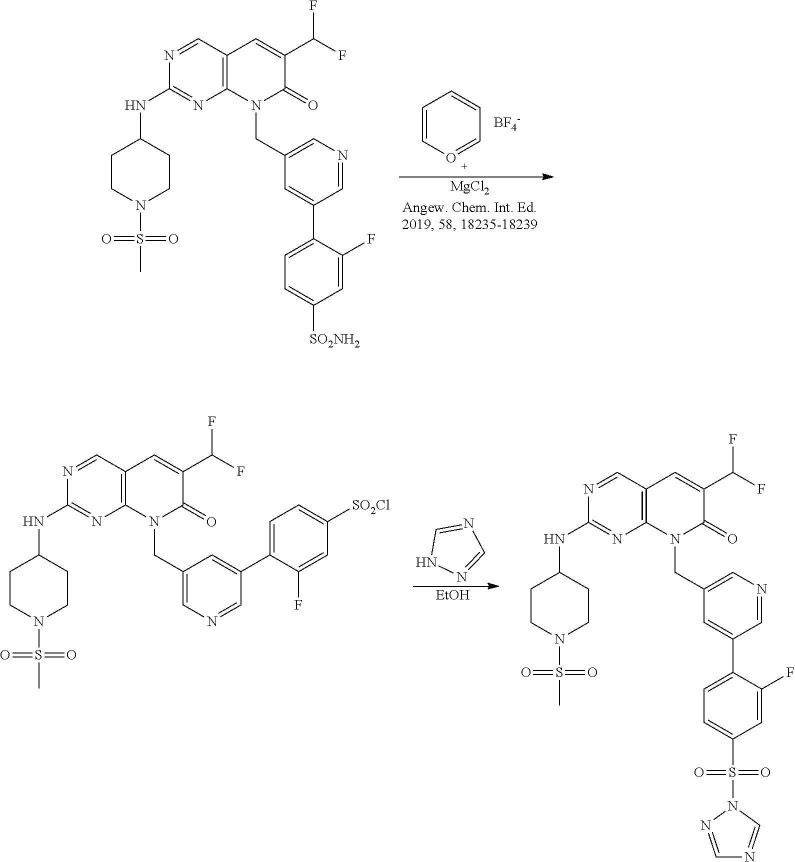

### Synthesis of Compound 204

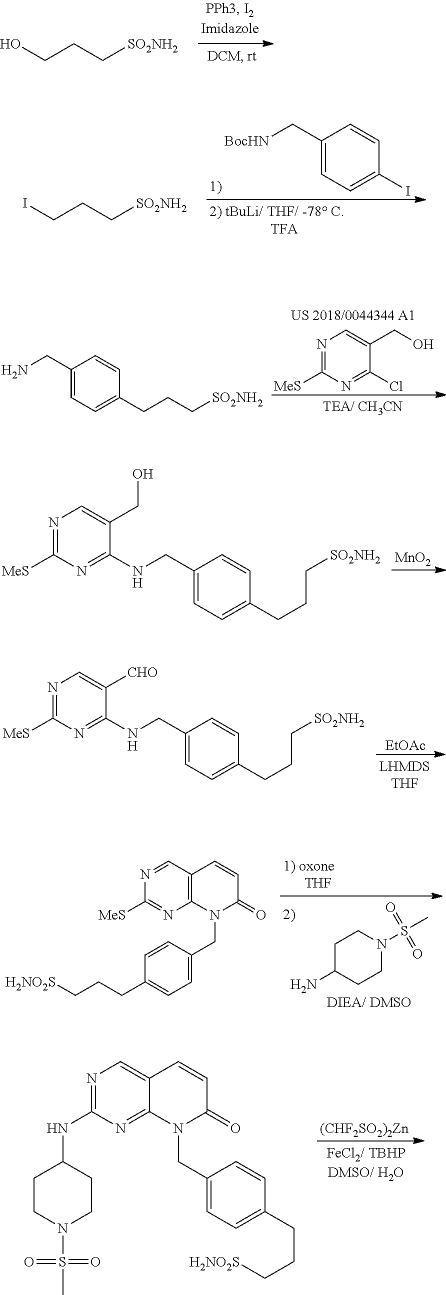

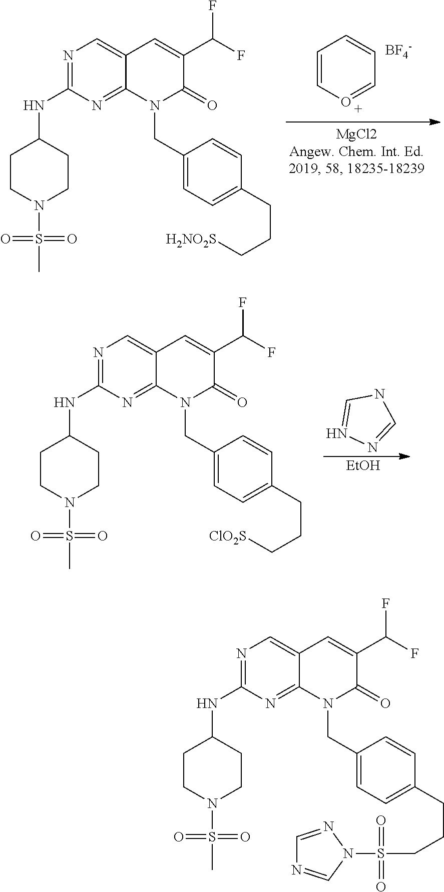

### Synthesis of Compound 205

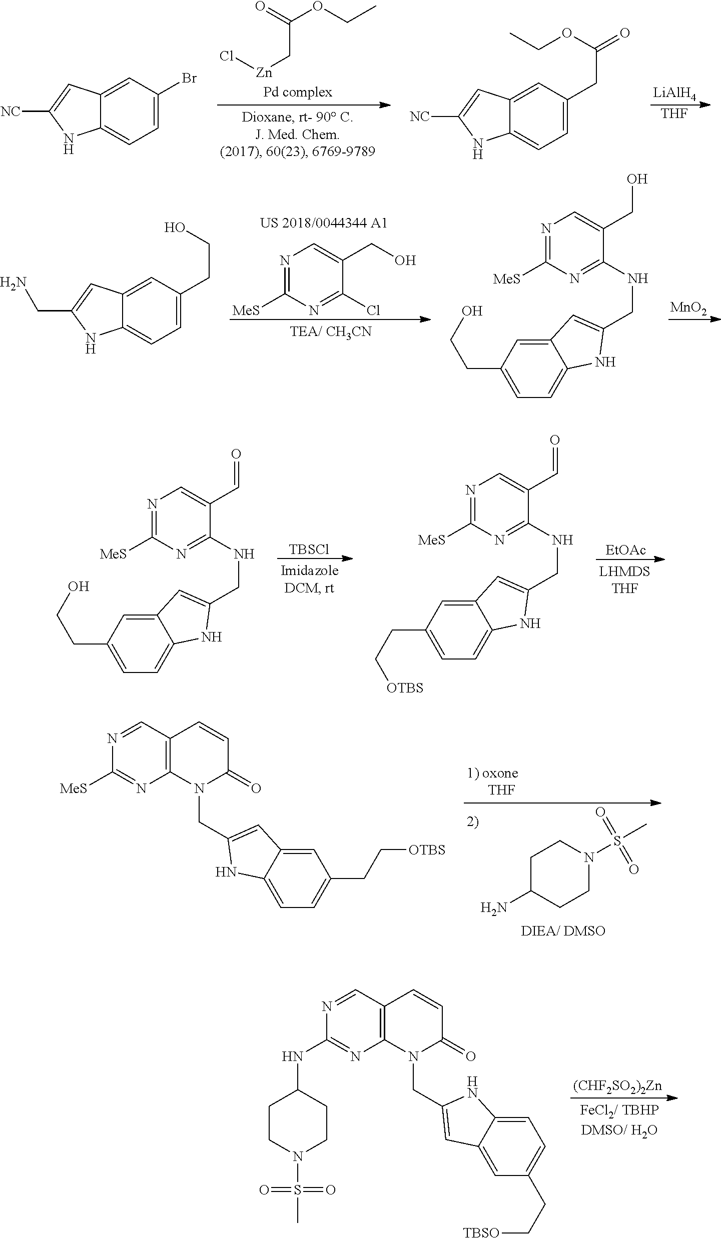

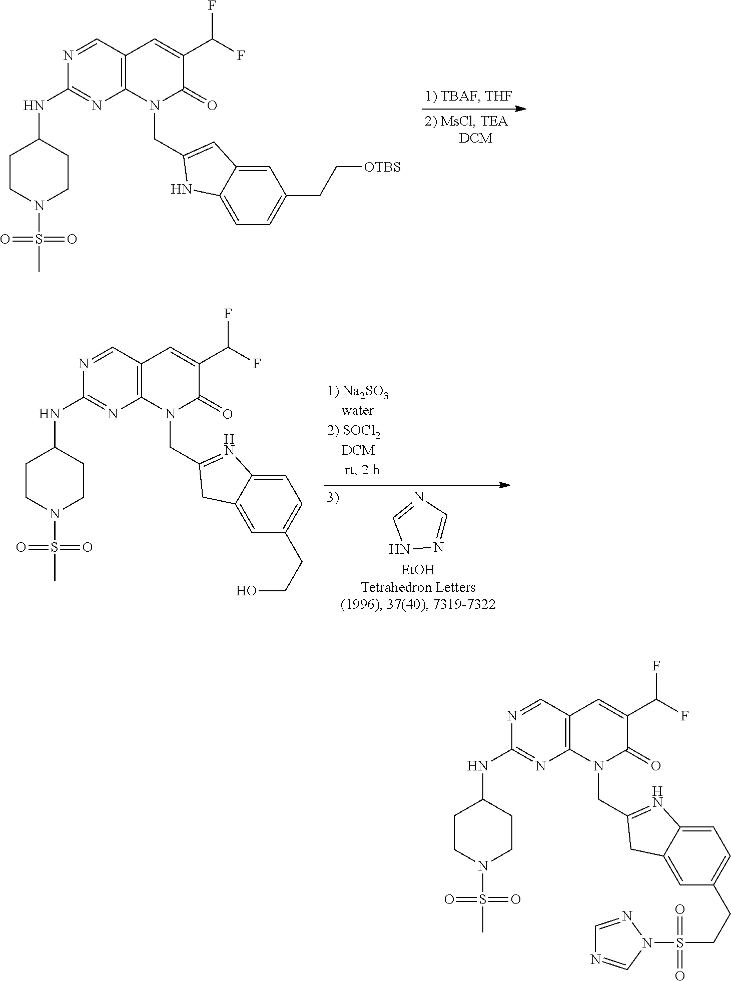

### Synthesis of Compound 206

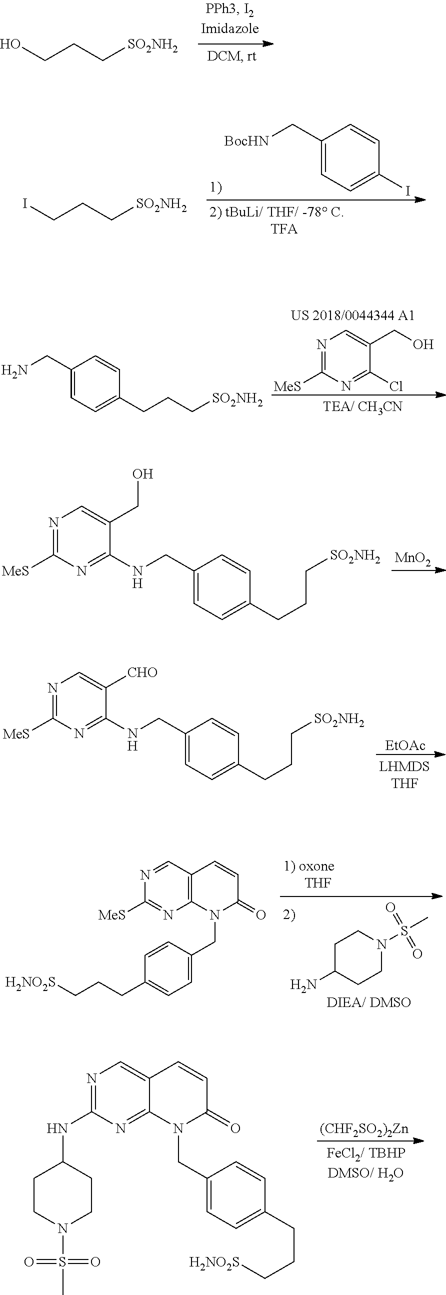

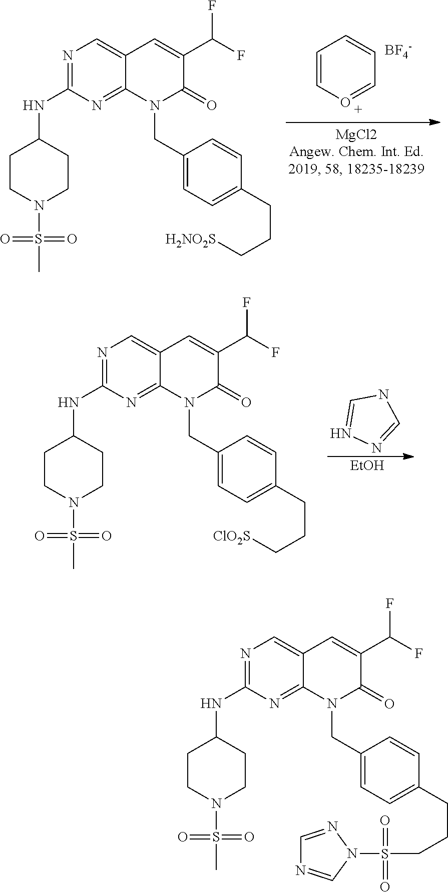

### Synthesis of Compound 207

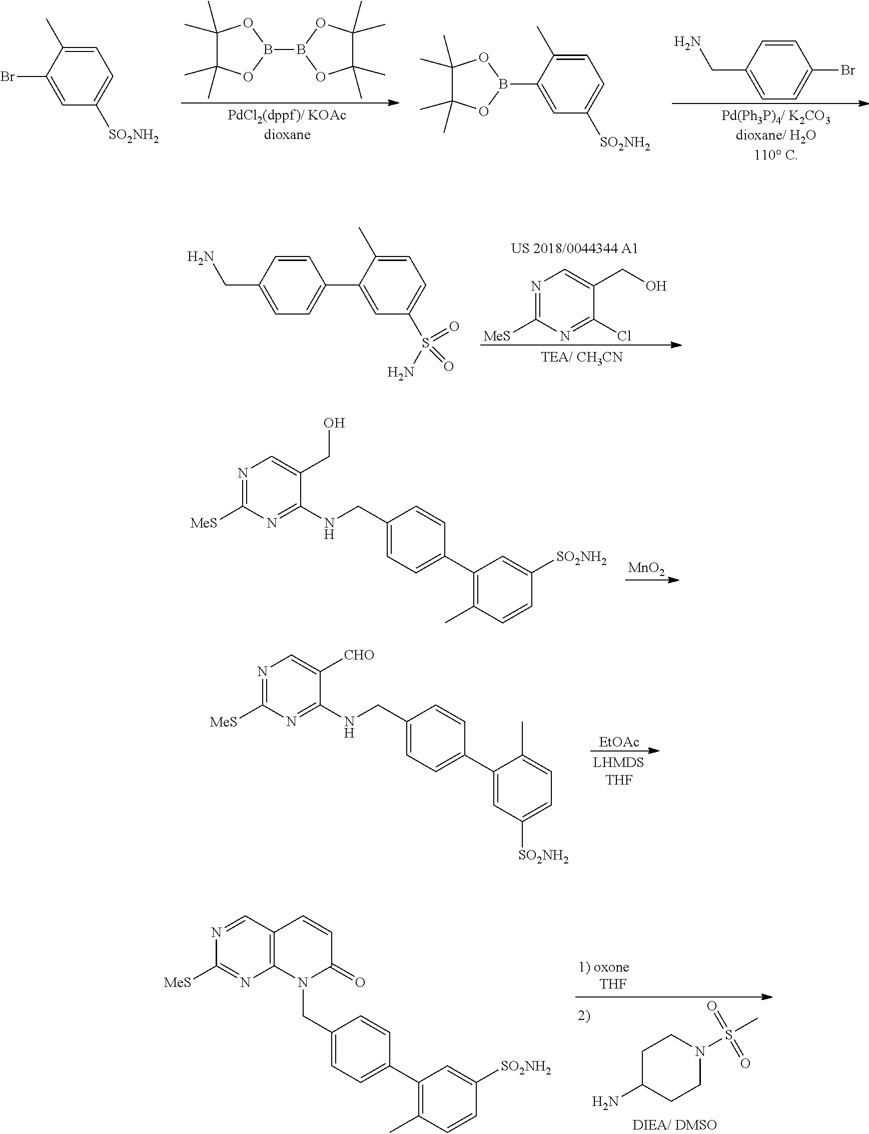

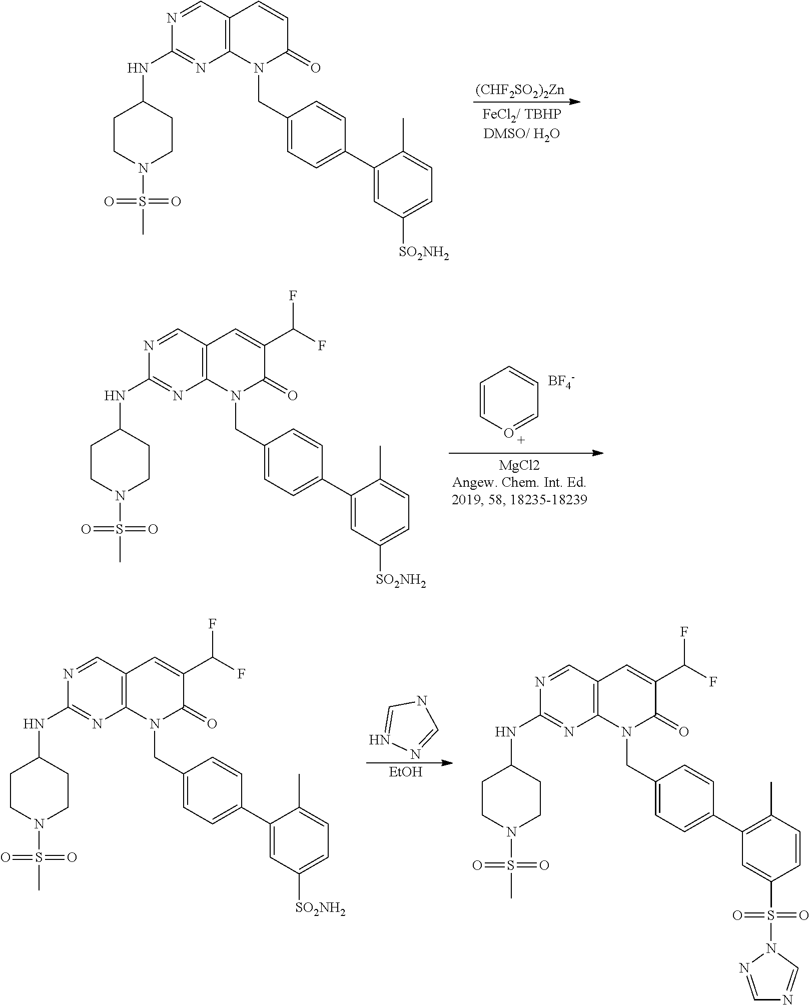

### Synthesis of Compound 208

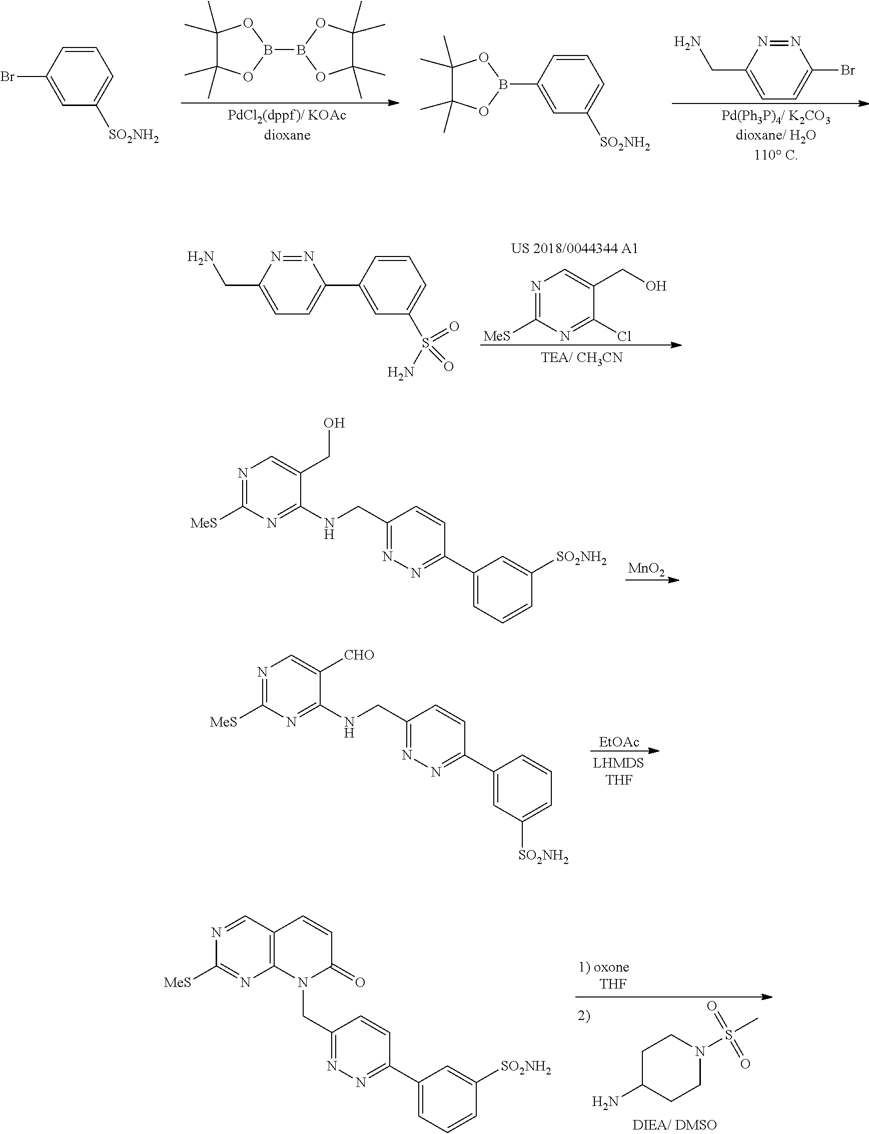

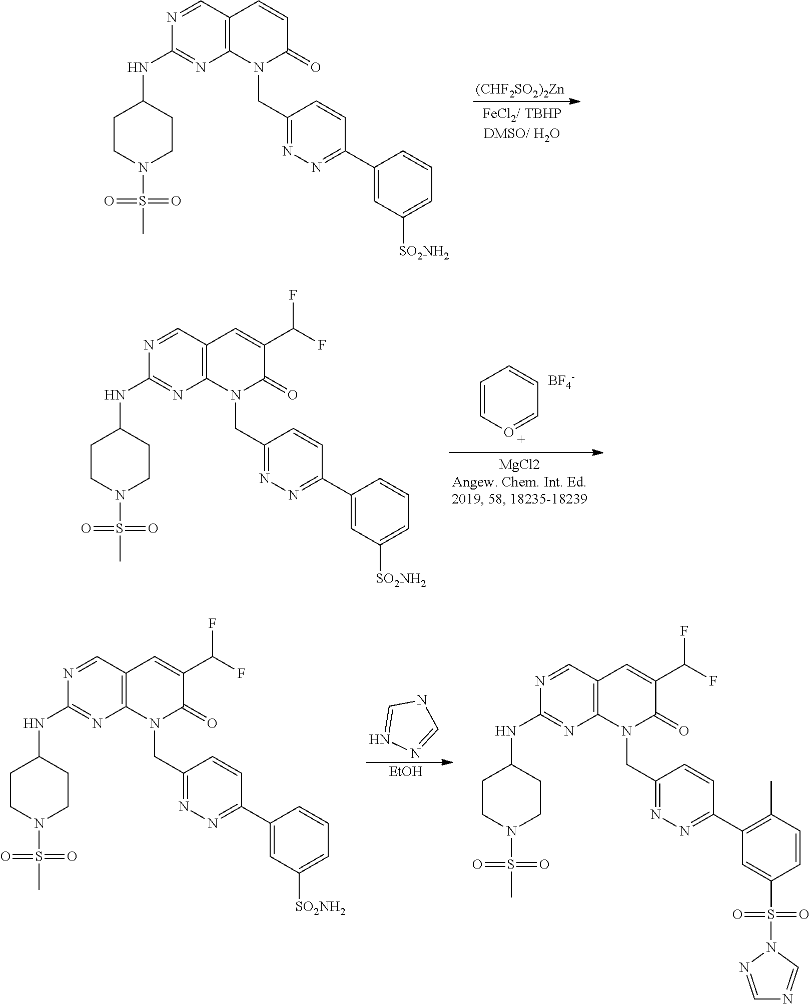

### Synthesis of Compound 209

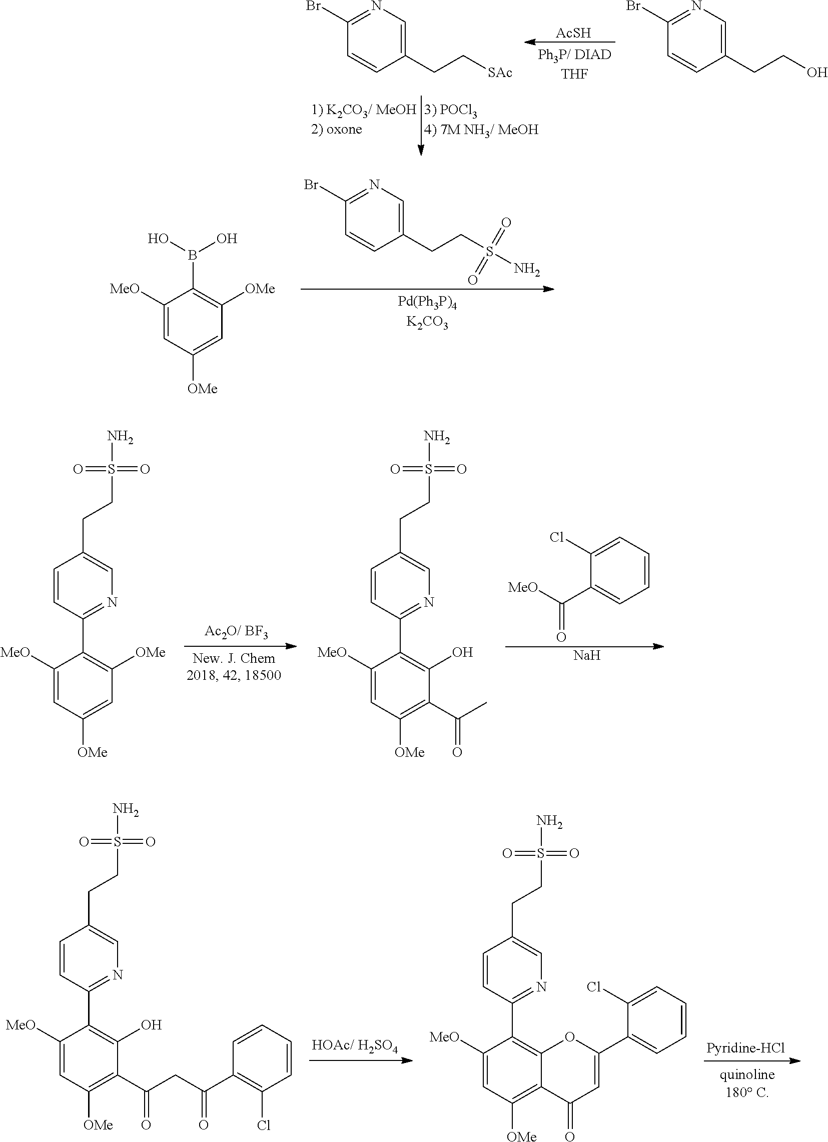

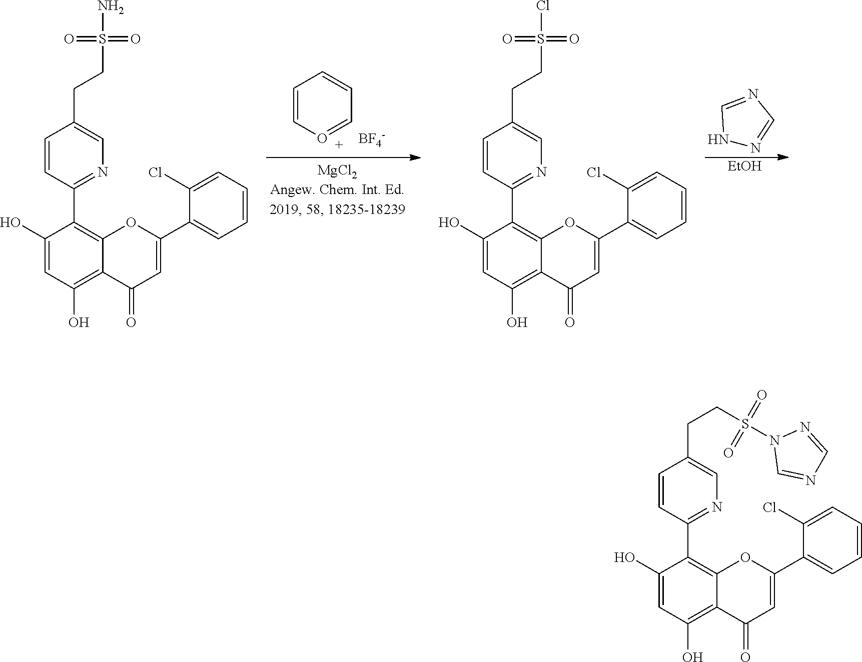

### Synthesis of Compound 210

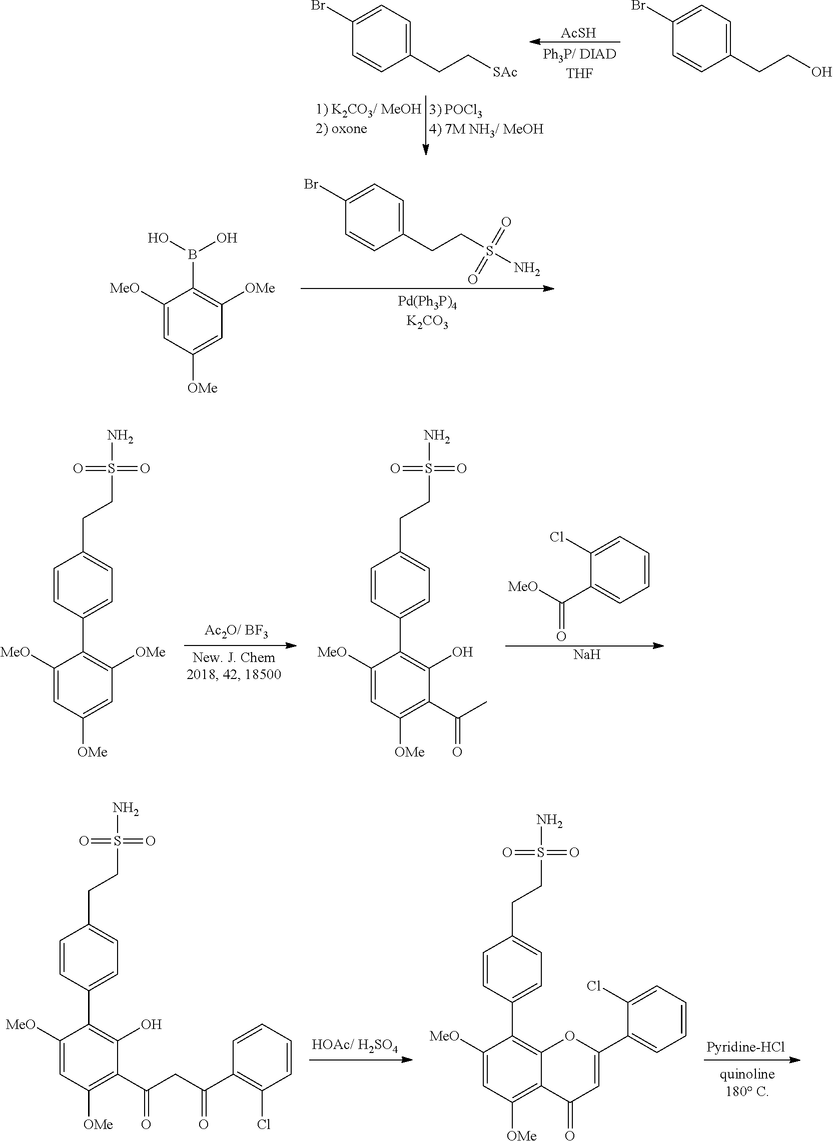

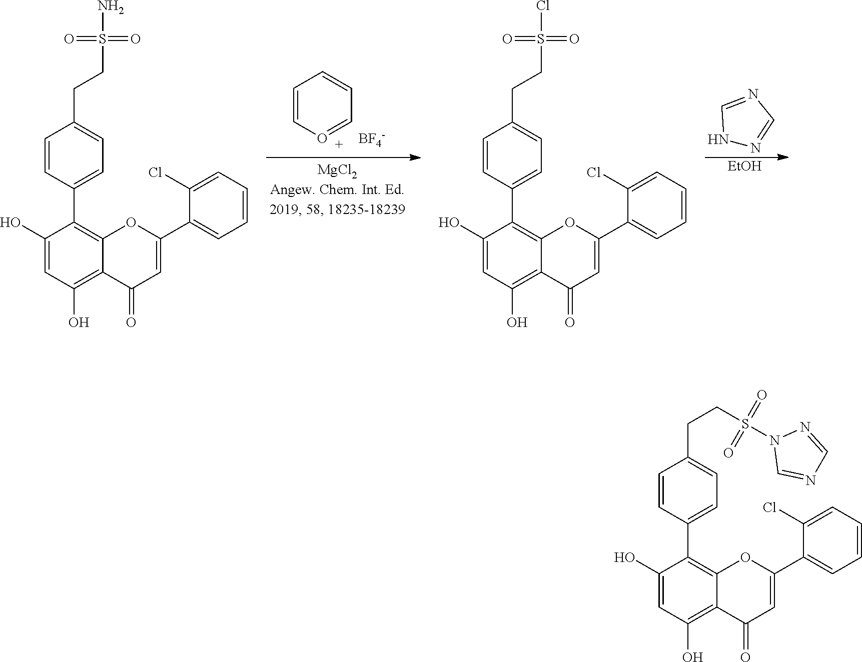

### Synthesis of Compound 211

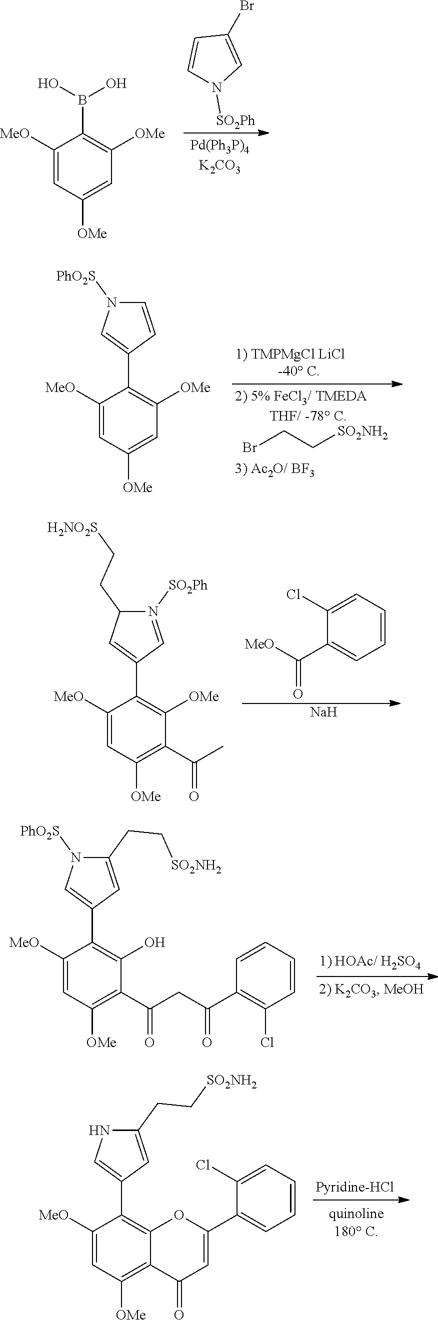

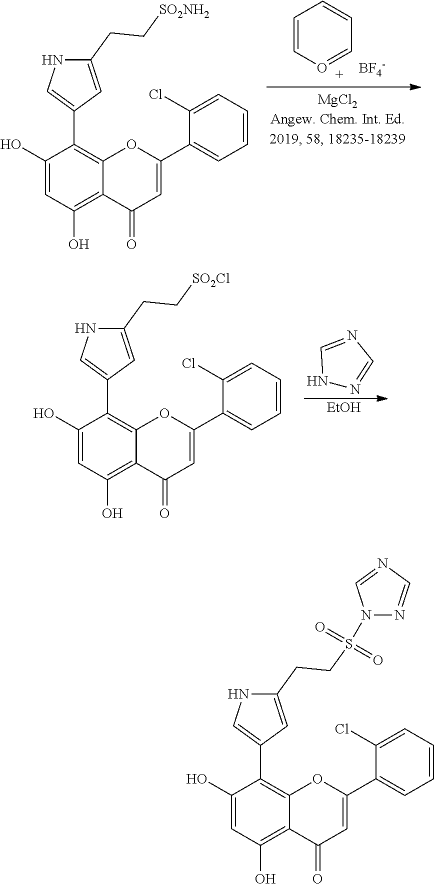

### Synthesis of Compound 212

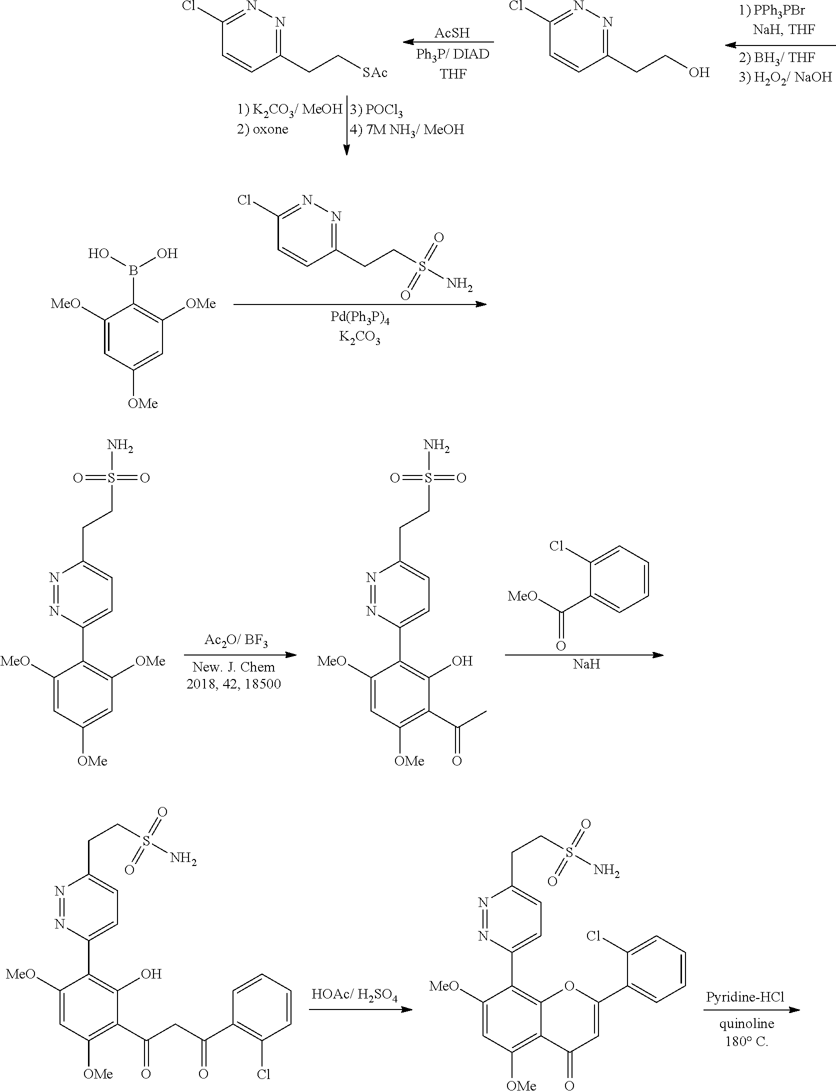

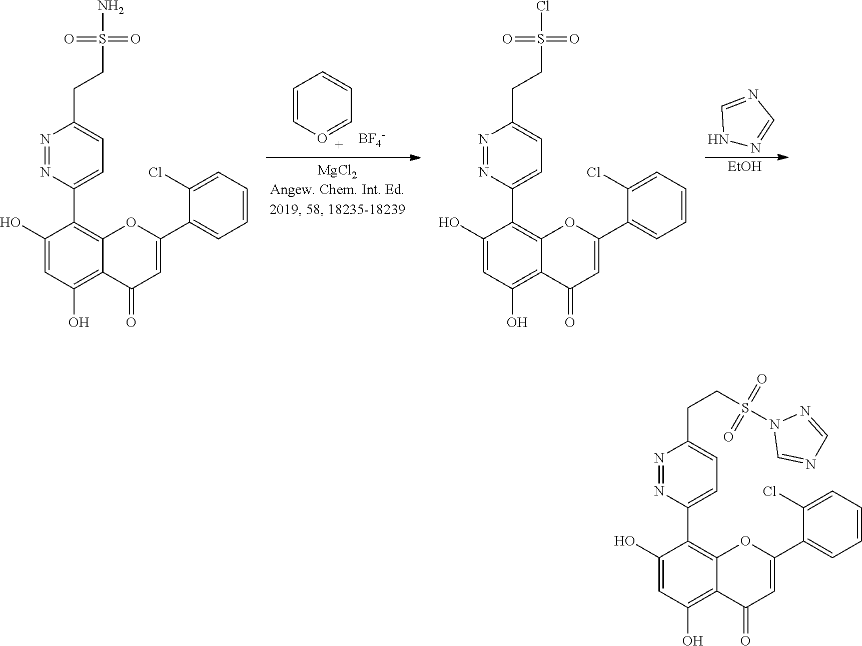

### Synthesis of Compound 213

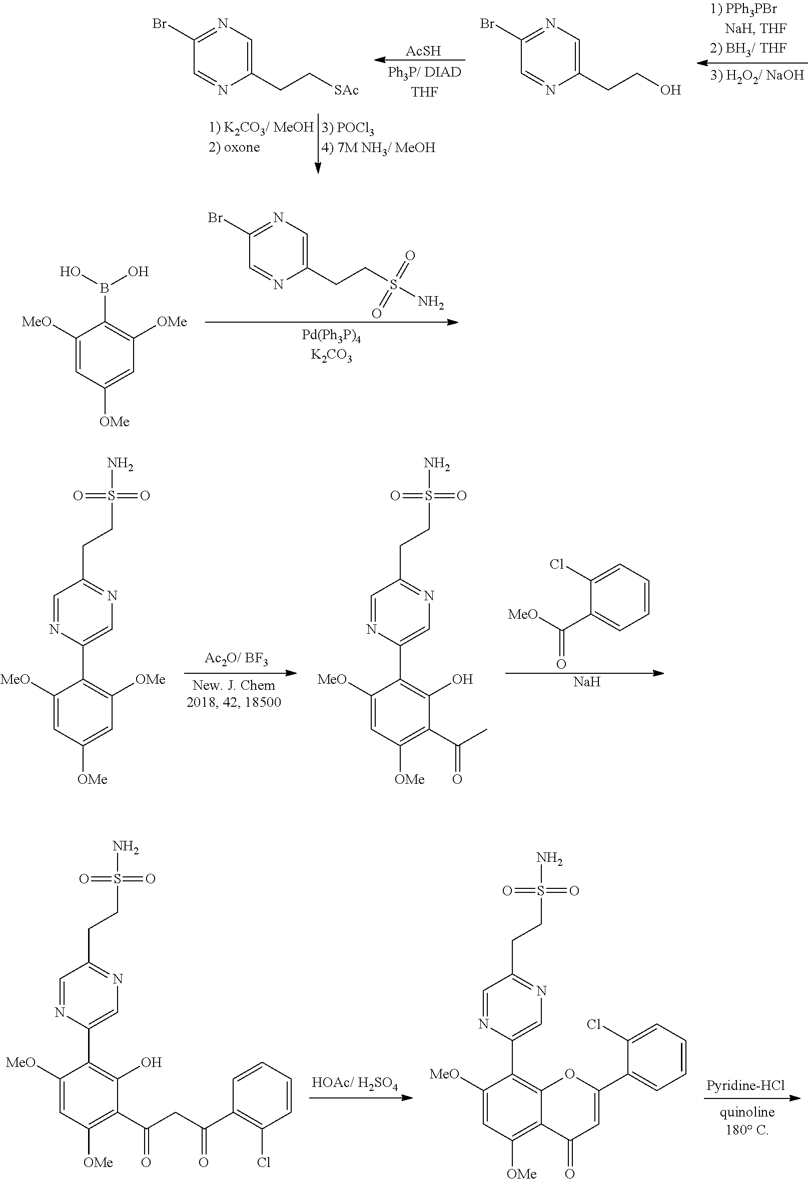

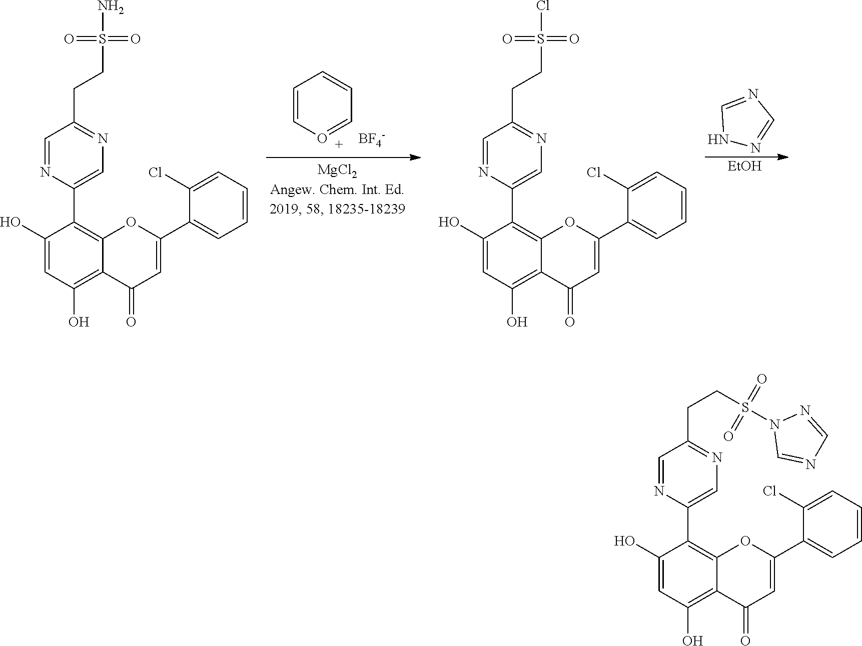

### Synthesis of Compound 214

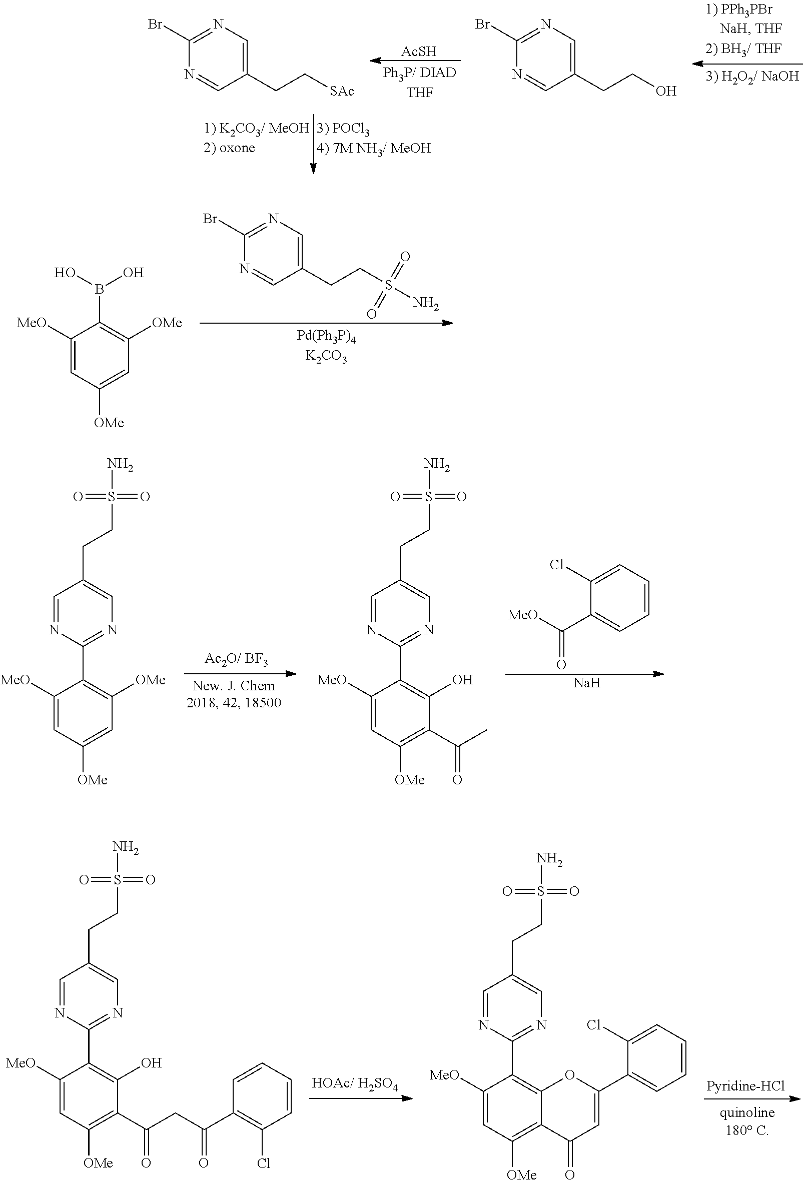

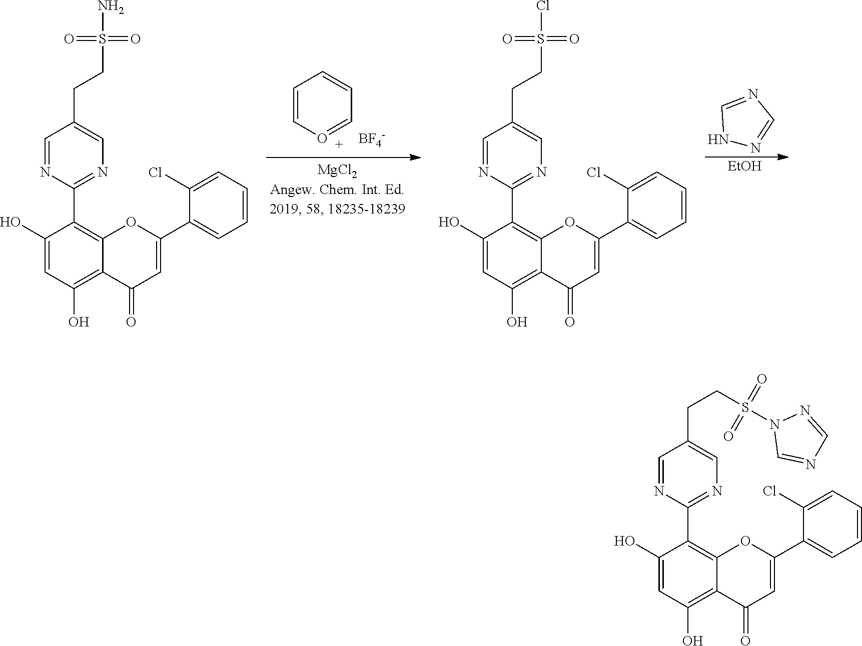

### Synthesis of Compound 215

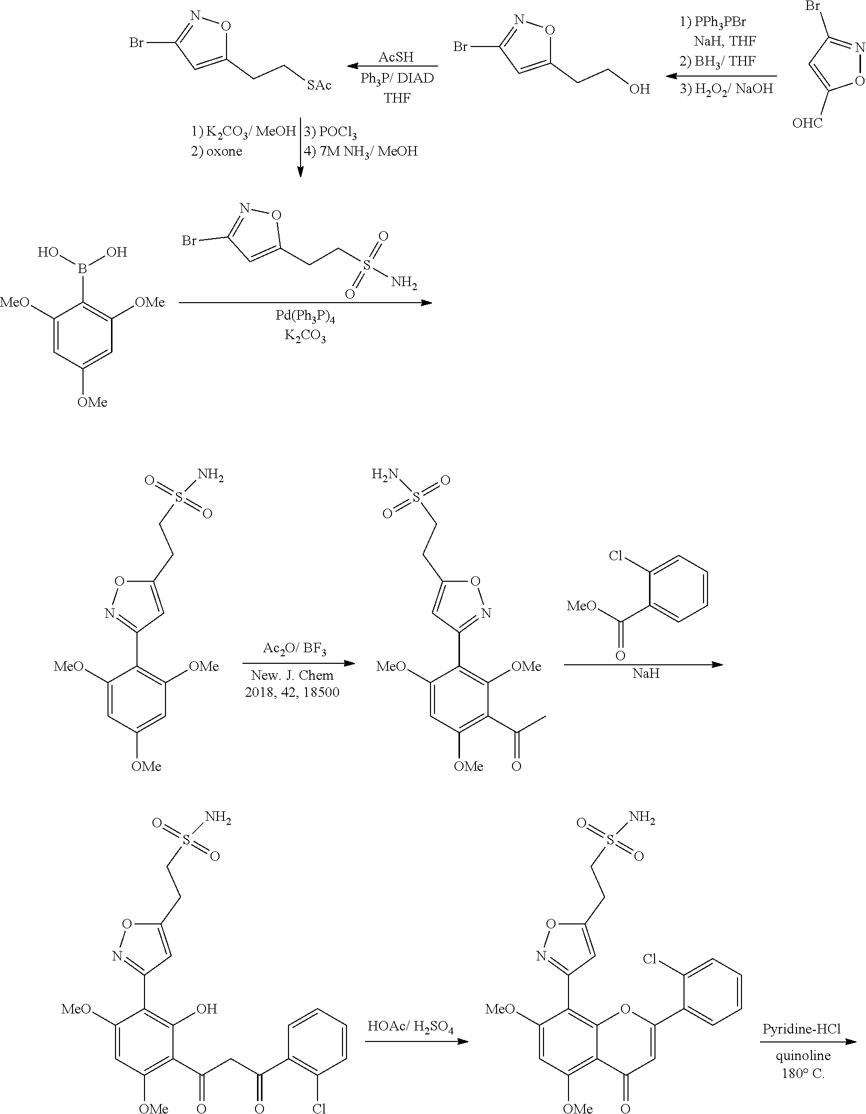

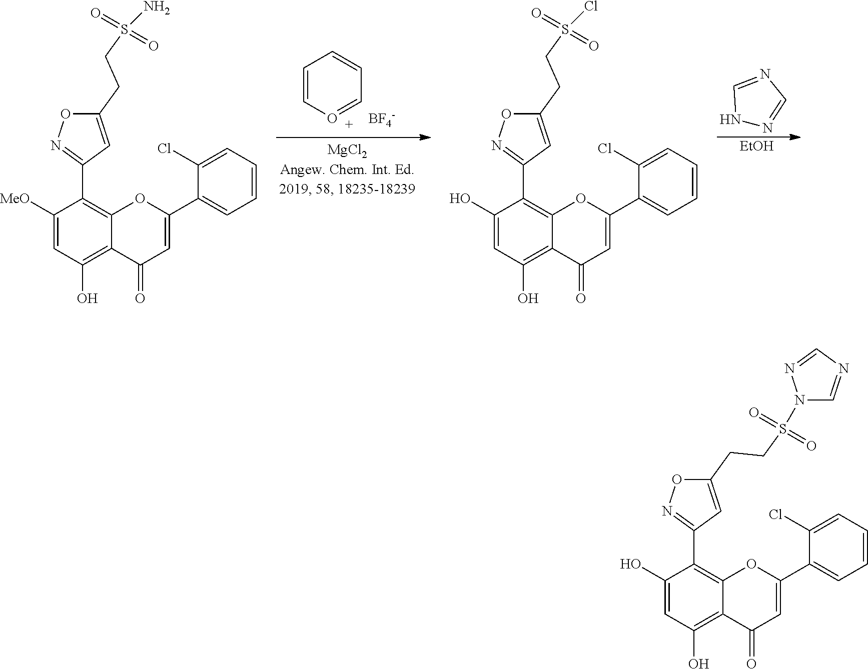

### Synthesis of Compound 216

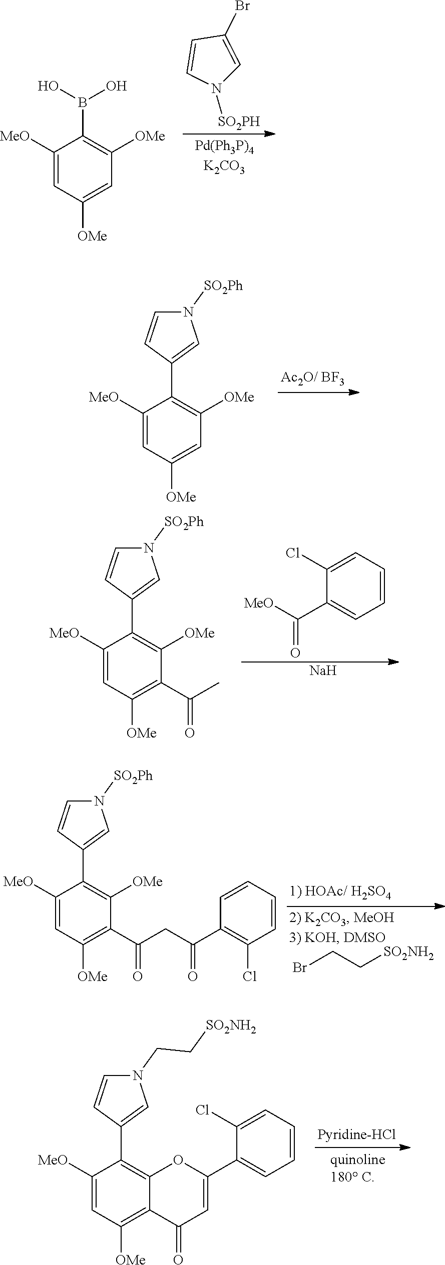

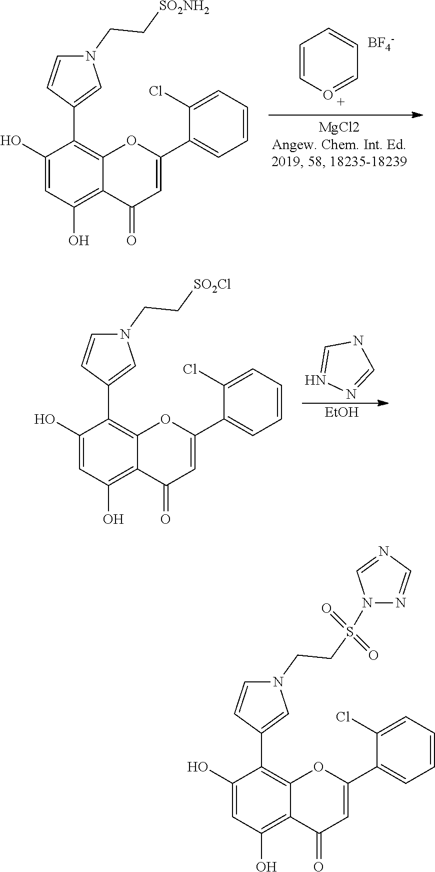

### Synthesis of Compound 217

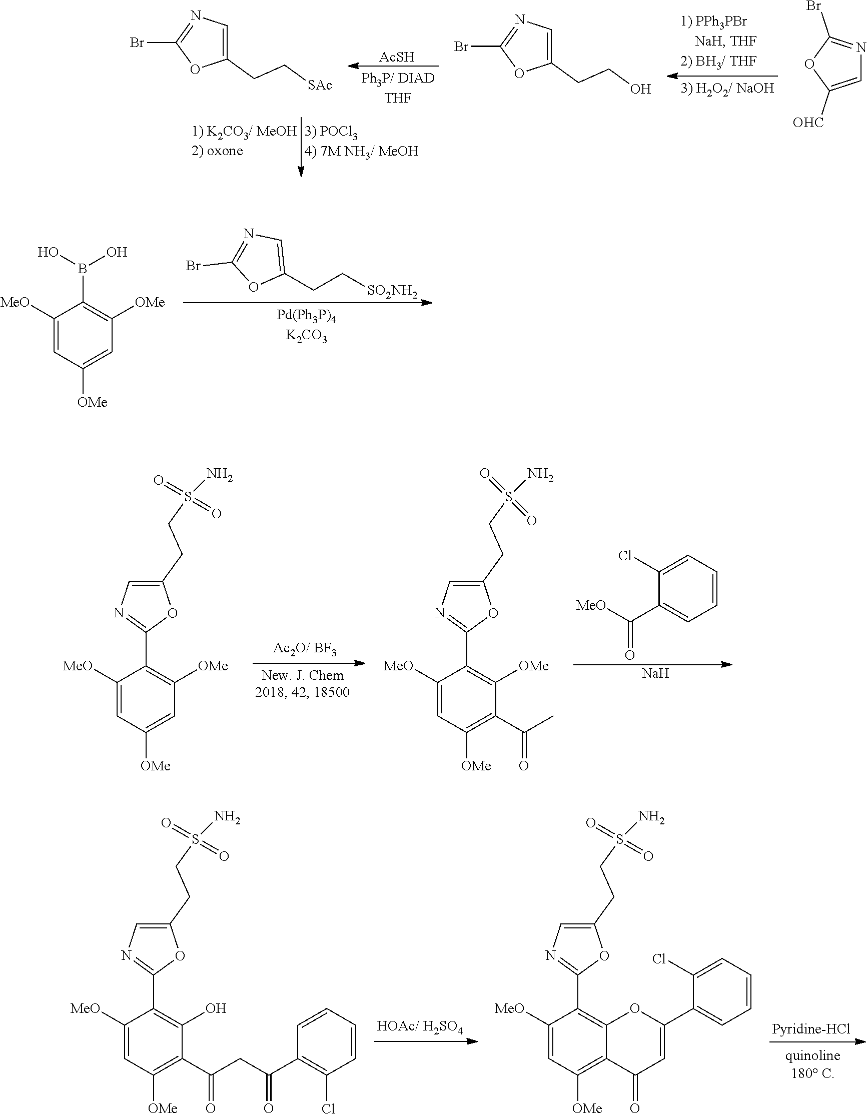

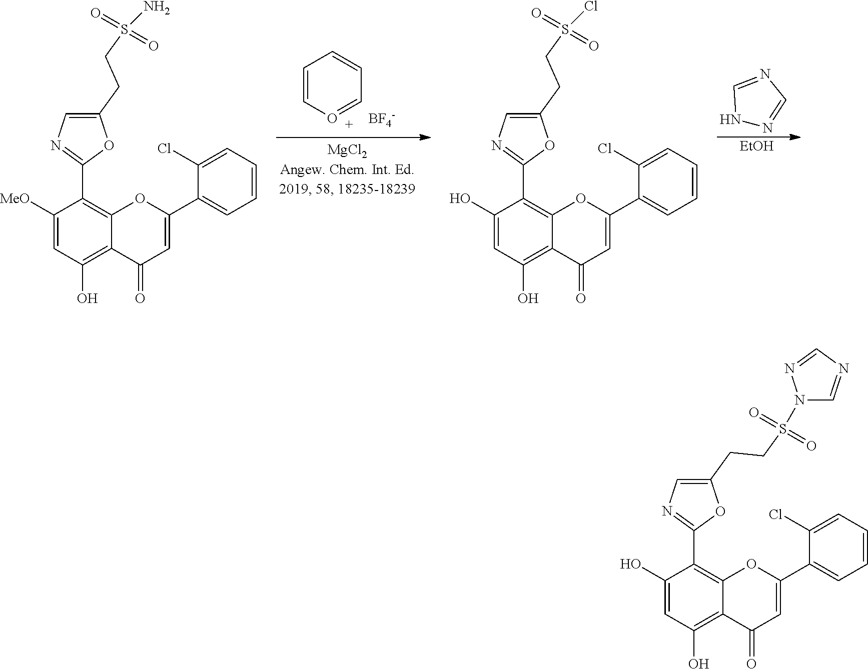

### Synthesis of Compound 218

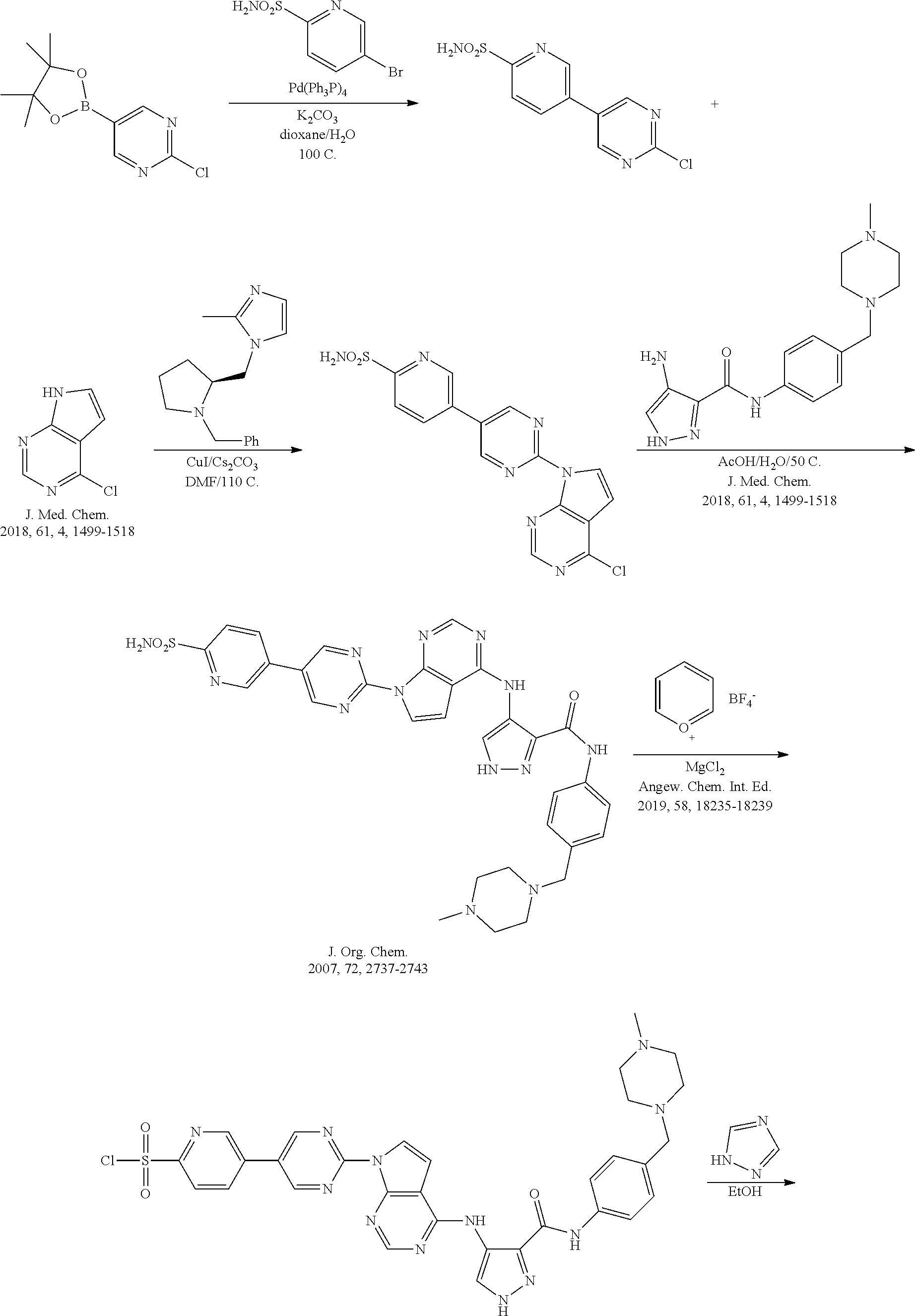

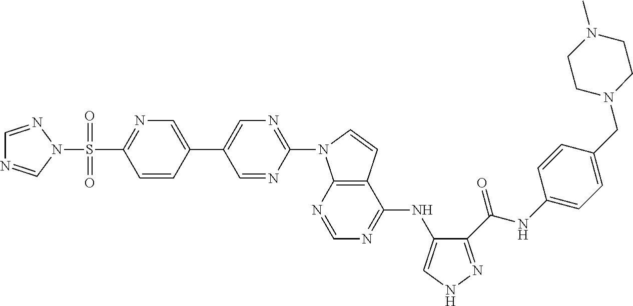 [SMILES: CN1CCN(Cc2ccc(NC(=O)c3n[nH]cc3Nc3ncnc4c3ccn4-c3ncc(-c4ccc(S(=O)(=O)n5cncn5)nc4)cn3)cc2)CC1]

### Synthesis of Compound 219

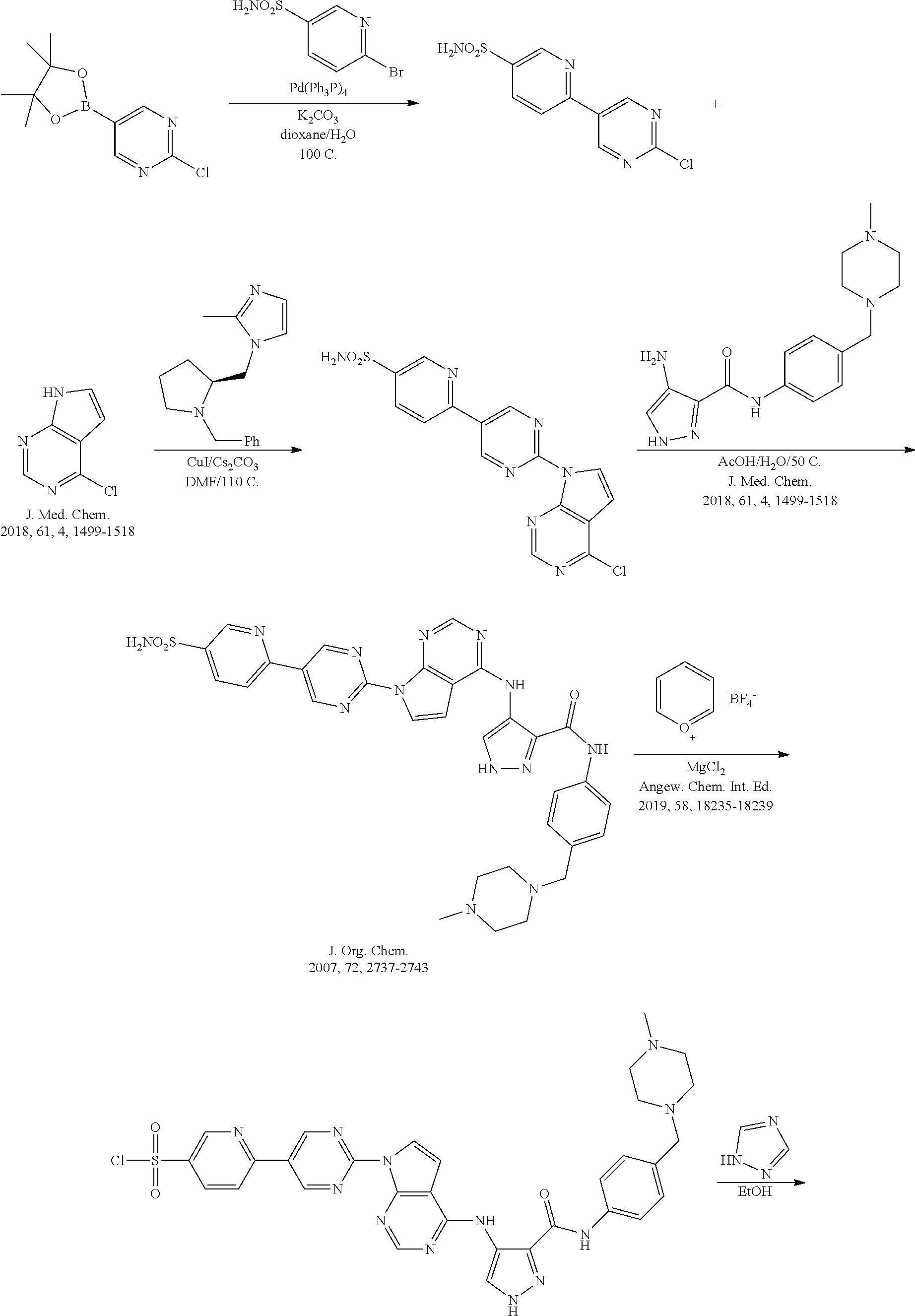

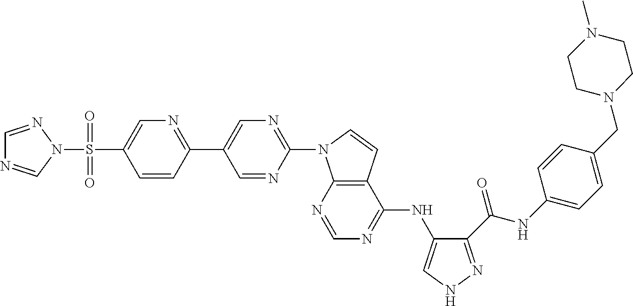 [SMILES: CN1CCN(Cc2ccc(NC(=O)c3n[nH]cc3Nc3ncnc4c3ccn4-c3ncc(-c4ccc(S(=O)(=O)n5cncn5)cn4)cn3)cc2)CC1]

### Synthesis of Compound 220

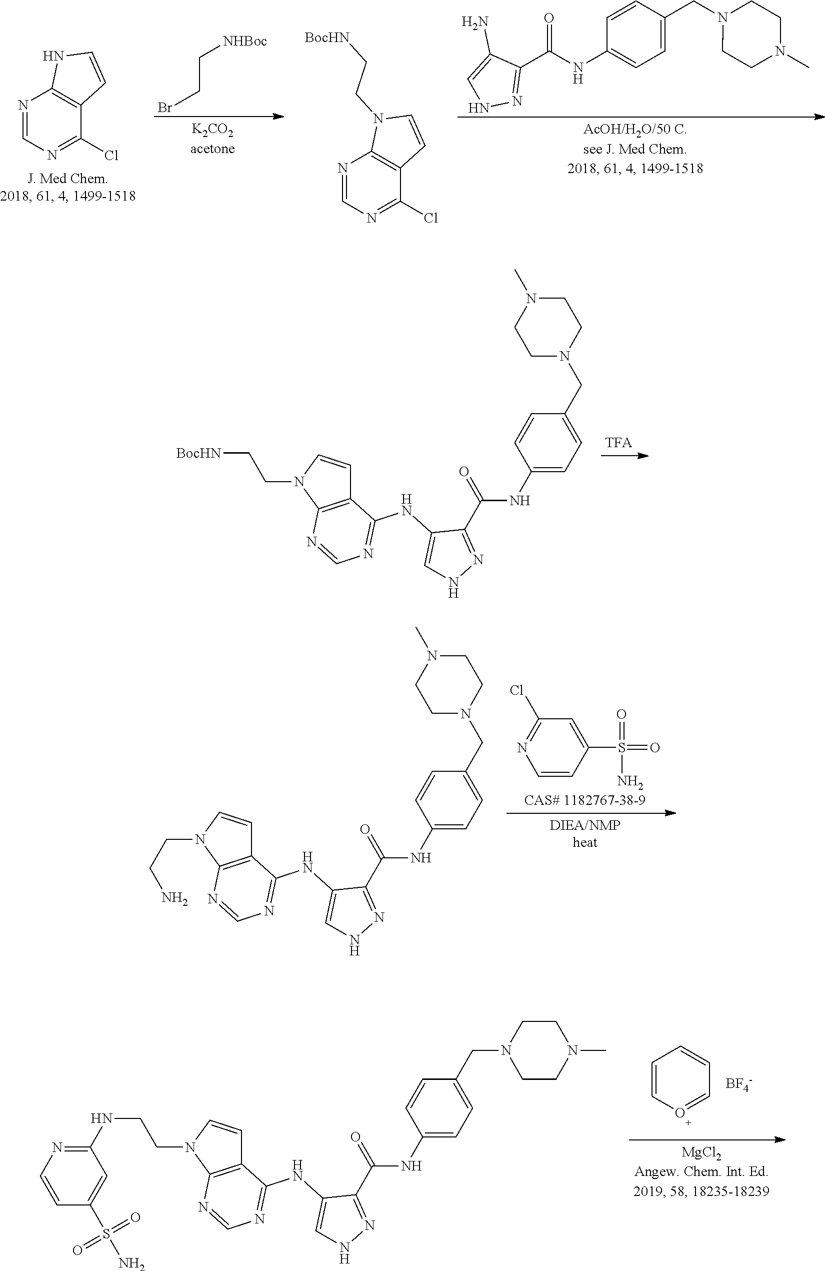

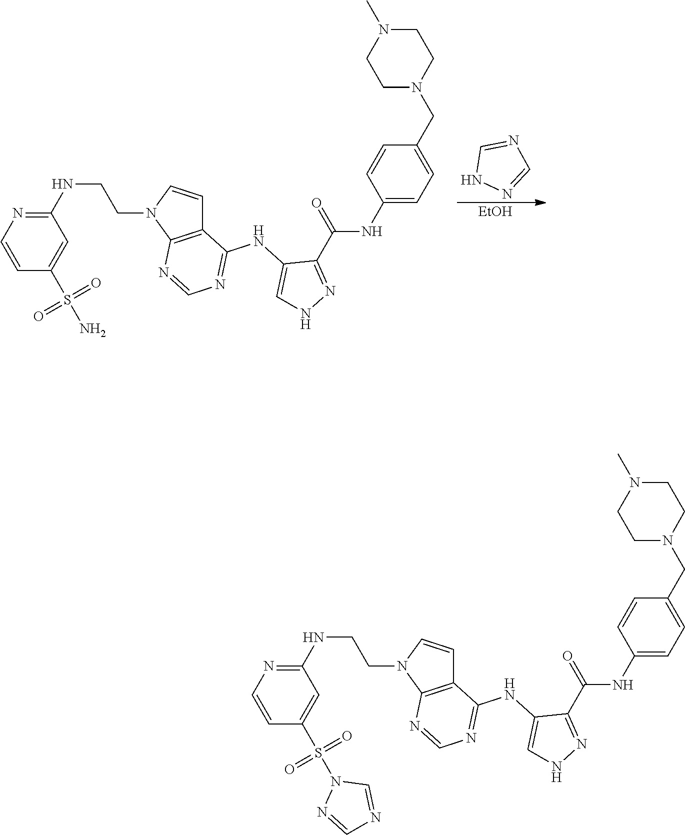

### Synthesis of Compound 221

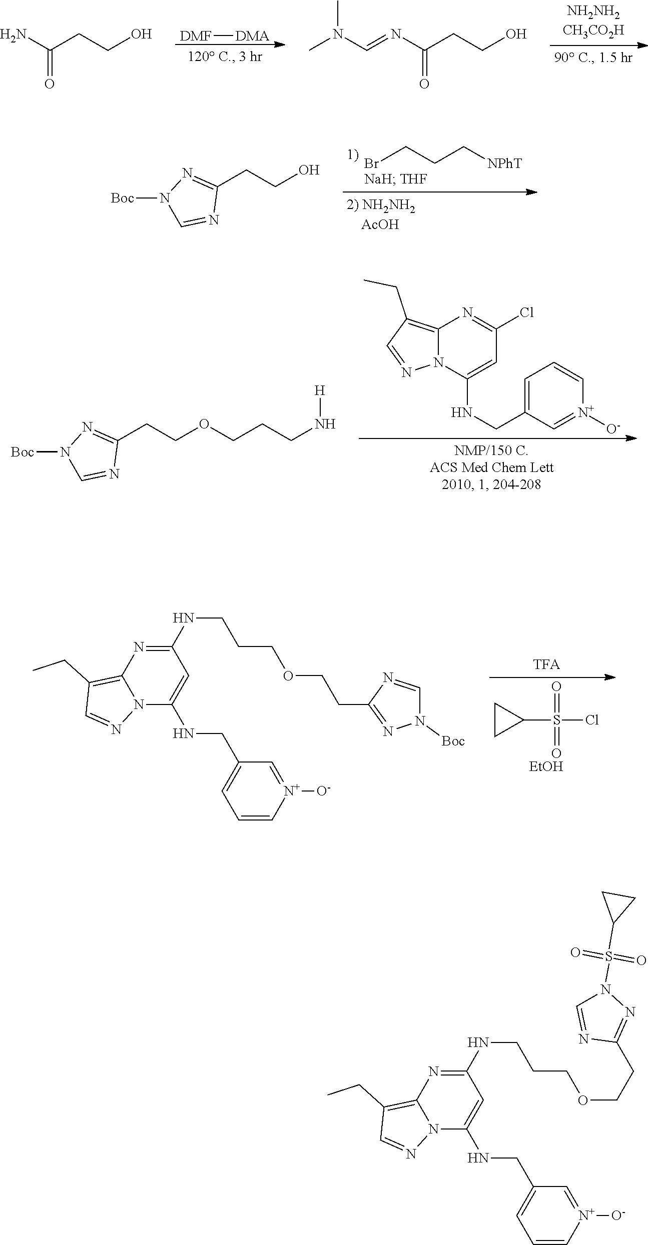

### Synthesis of Compound 222

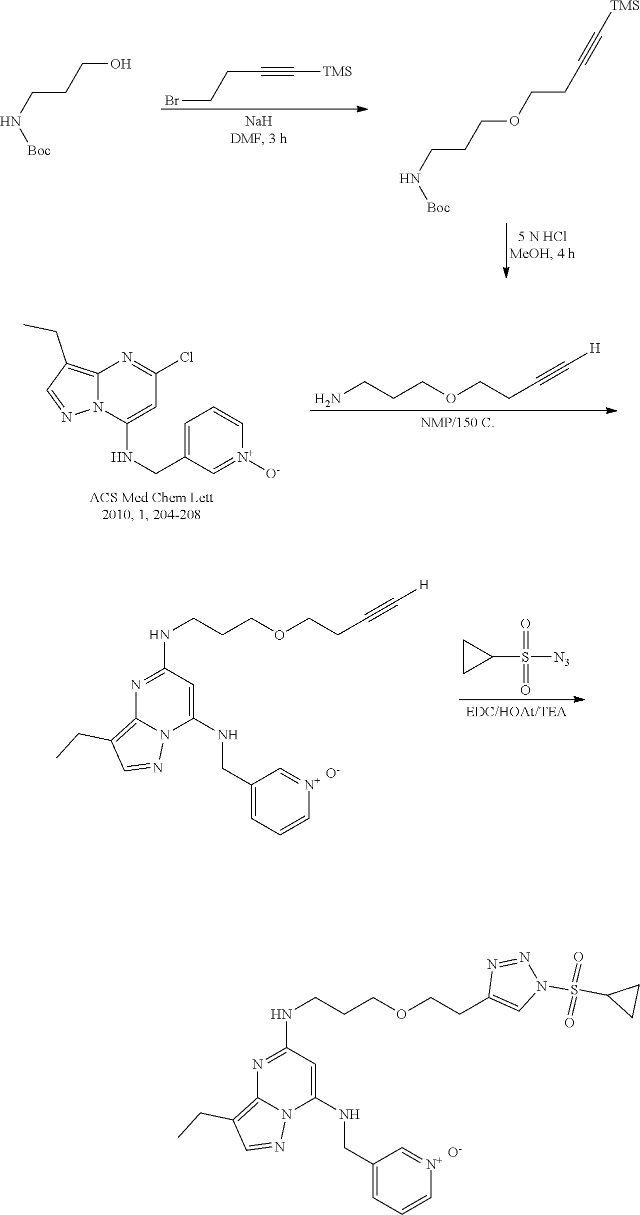

### Synthesis of Compound 223

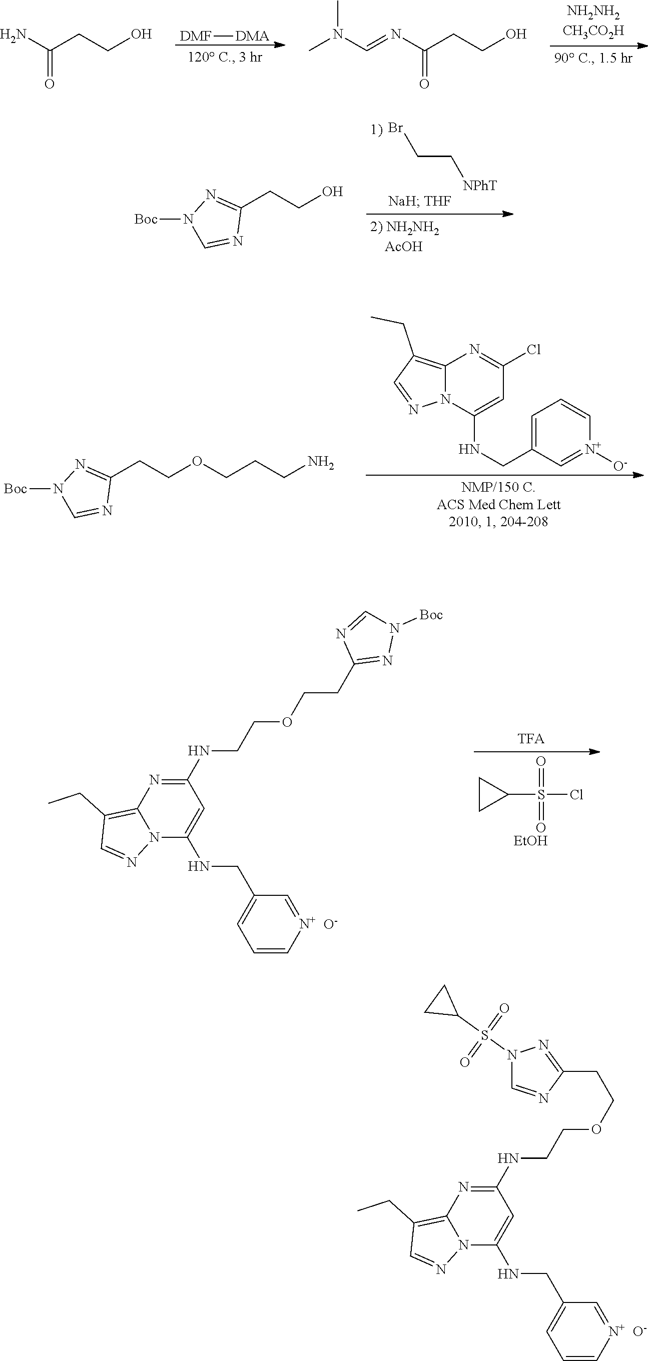

### Synthesis of Compound 224

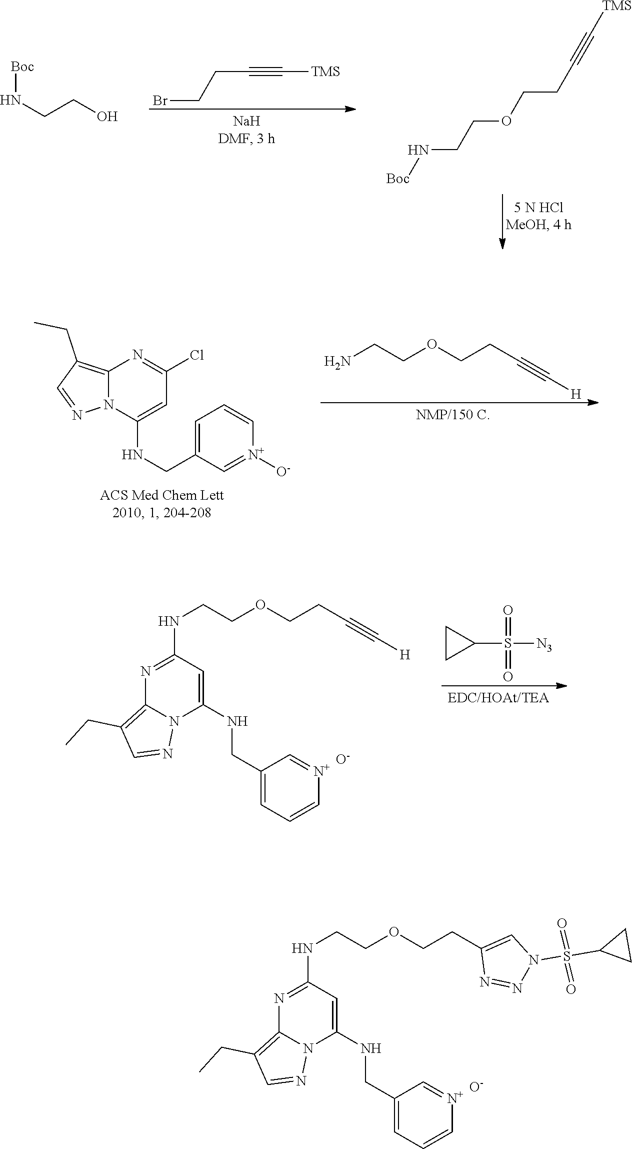

### Synthesis of Compound 225

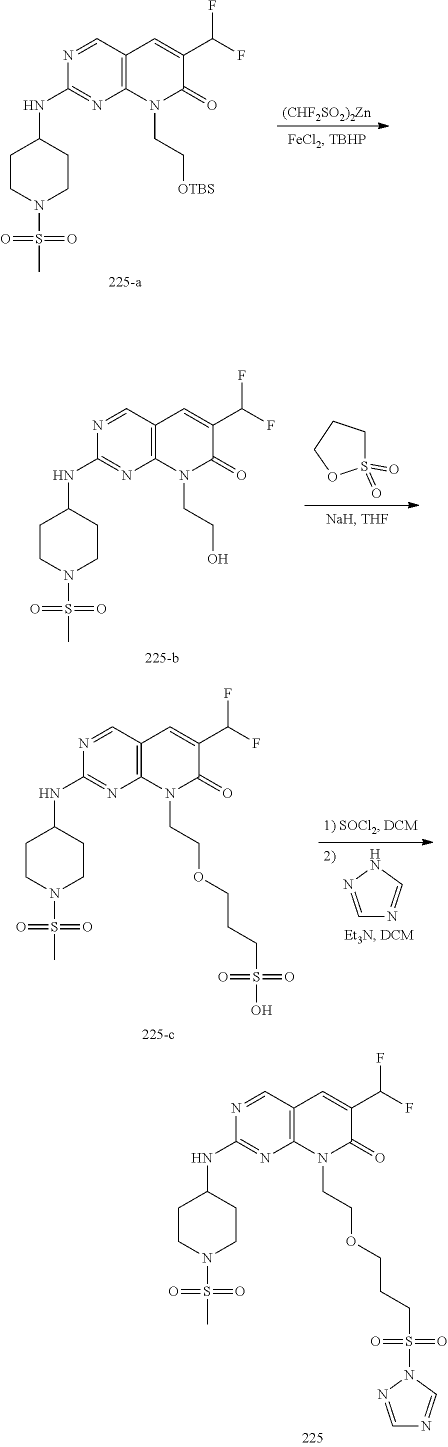

### Step 1

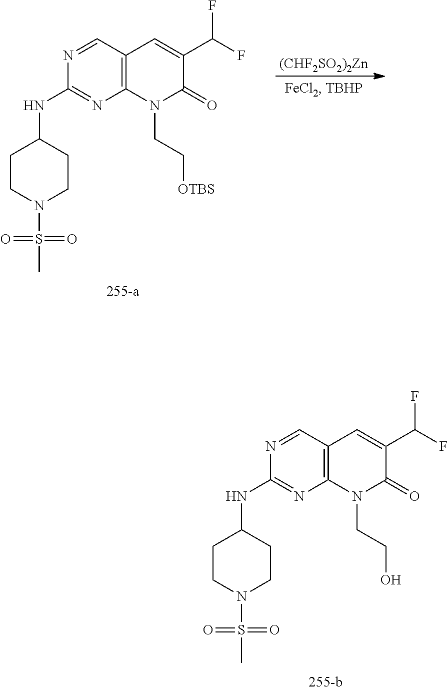

A solution of Zinc difluoromethanesulfinate (3 g, 10.15 mmol) and FeCl₂(0.1 g, 0.79 mmol) in H₂O (6 mL) was added portion-wise to a solution of 225-a (1.5 g, 3.11 mmol) in DMSO (5 mL) at rt. Then TBHP (0.28 g, 6.09 mmol) was added. After stirred at 25° C. for 18 h, a second portion of TBHP (0.14 g, 3.11 mmol) was added and stirring continued for 24 h at rt. The solution was slowly poured into water and extracted with EtOAc (30 mL×3). The organic layer was washed with brine (30 mL×2), dried over anhydrous Na₂SO₄, filtered and concentrated in vacuo. The residue was purified by silica gel flash chromatography (gradient 0%-10% MeOH in DCM) to afford 225-b (400 mg, 31% yield) as a white solid. m/z: [M+H]⁺ Calcd for C₁₆H₂₁F₂N₅O₄S 417.1; Found 417.8.

### Step 2

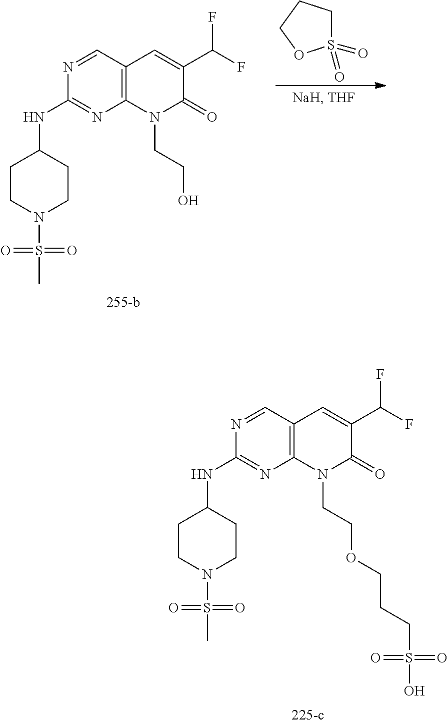 [SMILES: CS(=O)(=O)N1CCC(Nc2ncc3cc(C(F)F)c(=O)n(CCO)c3n2)CC1 ; O=S1(=O)CCCO1 ; CS(=O)(=O)N1CCC(Nc2ncc3cc(C(F)F)c(=O)n(CCOCCCS(=O)(=O)O)c3n2)CC1]

To a stirred solution of 225-b (50 mg, 0.12 mmol) in THF (10 mL) was added NaH (60% in oil) (10 mg, 0.25 mmol) under N2. After stirring at rt for 3 h, the solution was quenched by water and concentrated in vacuo to afford 225-c (70 mg, 35% yield) as a yellow solid. m/z: [M−H]⁻ Calcd for C₁₉H₂₇F₂N₅O₇S₂ 539.1; Found 537.8.

### Step 3

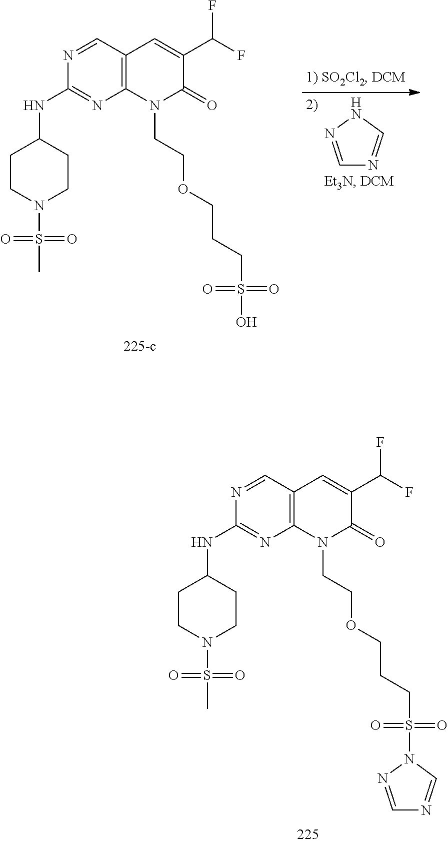 [SMILES: CS(=O)(=O)N1CCC(Nc2ncc3cc(C(F)F)c(=O)n(CCOCCCS(=O)(=O)O)c3n2)CC1 ; c1nc[nH]n1 ; CS(=O)(=O)N1CCC(Nc2ncc3cc(C(F)F)c(=O)n(CCOCCCS(=O)(=O)n4cncn4)c3n2)CC1]

To a solution of 225-c (50 mg, 0.028 mmol) in toluene (10 mL) was added SOCl₂(0.2 mL, 2.76 mmol) with stirring at rt. After stirred at 90° C. for 1 h, the reaction mixture was concentrated and the residue was dissolved into DCM (1 mL), a solution of 1H-1,2,4-triazole (0.002 mL, 0.028 mmol) and Et₃N (10 mg, 0.099 mmol) in DCM (6 ml) was added. Then the mixture was stirred for 1 h and purified by preparative thin-layer chromatography (70% EtOAc in hexanes) and reverse-phase chromatography (SilaSep™ C18 silica flash cartridge, 20%-60% MeCN in H₂O with 0.1% formic acid) to afford 225 (1.5 mg, 9% yield) as a white solid. m/z: [M+H]⁺ Calcd for C₂₁H₂₈F₂N₈O₆S₂ 590.1; Found 591.5. ¹H NMR (400 MHz, CDCl₃) δ 8.67 (s, 1H), 8.55 (s, 1H), 8.14 (s, 1H), 7.85 (s, 1H), 6.79 (t, J=55.4 Hz, 1H), 4.52 (d, J=35.2 Hz, 2H), 4.06 (d, J=44.6 Hz, 1H), 3.75 (s, 4H), 3.56 (s, 4H), 2.93 (d, J=68.9 Hz, 6H), 2.21 (d, J=11.0 Hz, 2H), 2.01-1.95 (m, 2H), 1.74 (s, 1H).

### Synthesis of Compound 226

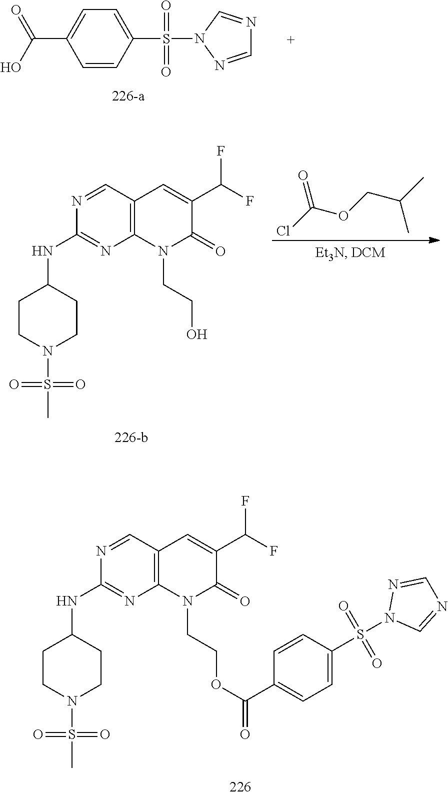

To a solution of 226-a (40 mg, 0.16 mmol) and Et₃N (70 mg, 0.69 mmol) in DCM (10 mL) was added chloro(2-methylpropoxy)methanone (0.029 mL, 0.22 mmol) with stirring at 0° C. After stirred at 0° C. for 1 h, 226-b (50 mg, 0.12 mmol) was added. Then the reaction mixture was stirred at rt for 1 h, concentrated and purified by silica gel flash chromatography (gradient 0%-100/0 EtOAc in hexanes) and reverse-phase chromatography (SilaSep™ C18 silica flash cartridge, 60%-90% MeCN in H₂O with 0.1% formic acid) to afford 226 (7 mg, 9% yield) as a yellow solid. m/z: [M+H]⁺ Calcd for C₂₅H₂₆F₂N₈O₇S₂ 652.1; Found 652.7. ¹H NMR (400 MHz, CDCl₃) δ 8.76 (s, 1H), 8.54 (s, 1H), 8.14-8.05 (m, 5H), 7.87 (s, 1H), 6.79 (t, J=55.6 Hz, 1H), 5.37 (d, J=117.0 Hz, 1H), 4.74 (d, J=29.2 Hz, 4H), 3.98 (s, 1H), 3.79 (d, J=22.0 Hz, 2H), 2.95 (s, 2H), 2.83 (s, 3H), 2.06 (d, J=11.0 Hz, 2H), 1.64 (s, 1H), 1.52 (s, 1H).

### Synthesis of Compound 227

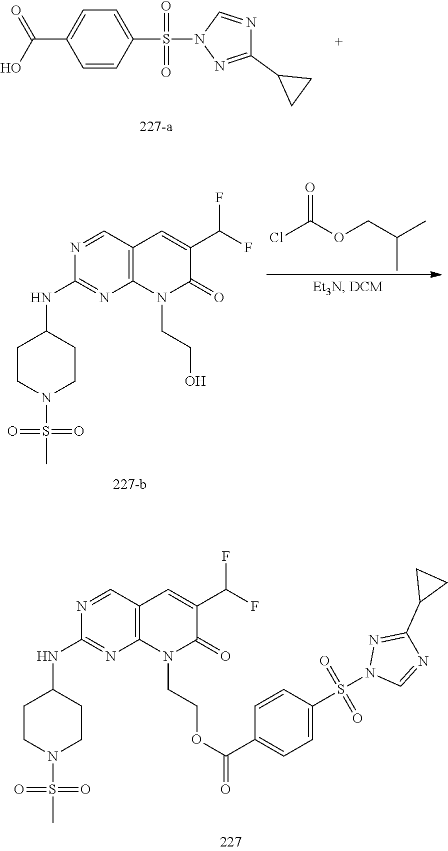

To a solution of 227-a (50 mg, 0.17 mmol) and Et₃N (30 mg, 0.30 mmol) in 10 mL of DCM was added chloro(2-methylpropoxy)methanone (30 mg, 0.22 mmol) with stirring at 0° C. After stirred at 0° C. for 1 h, 227-b (50 mg, 0.12 mmol) was added. Then the reaction mixture was stirred at rt for 1 h, concentrated and purified by silica gel flash chromatography (gradient 0%-100% EtOAc in hexanes) and reverse-phase chromatography (SilaSep™ C18 silica flash cartridge, 20%-60% MeCN in H₂O with 0.1% formic acid) to afford 227 (15 mg, 18% yield) as a yellow solid. m/z: [M+H]⁺ Calcd for C₂₈H₃₀F₂N₈O₇S₂ 692.1; Found 693.5. ¹H NMR (400 MHz, CDCl₃) δ 8.54 (d, J=5.6 Hz, 2H), 8.08 (s, 4H), 7.88 (s, 1H), 6.79 (t, J=55.3 Hz, 1H), 5.38 (d, J=111.3 Hz, 1H), 4.74 (d, J=27.8 Hz, 4H), 3.86 (m, 3H), 2.88 (m, 5H), 2.04 (d, J=6.1 Hz, 3H), 1.65 (m, 2H), 1.21 (d, J=22.0 Hz, 1H), 1.01-0.97 (m, 3H).

### Synthesis of Compound 228

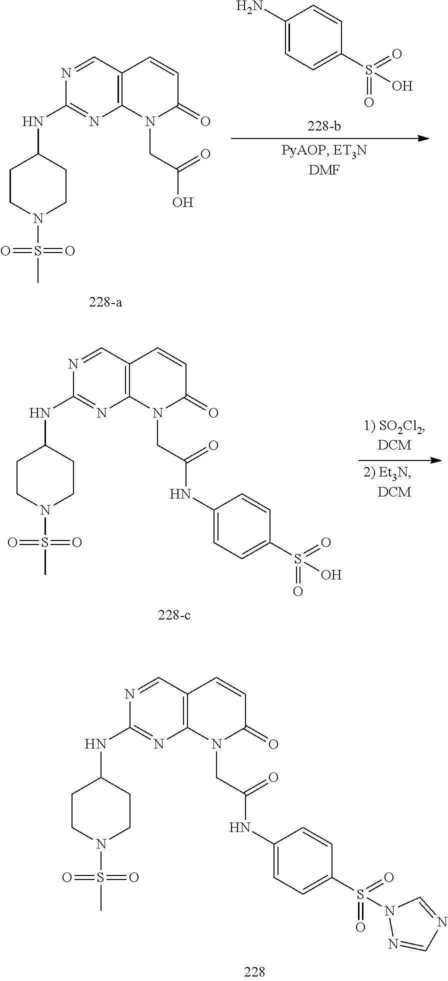 [SMILES: CS(=O)(=O)N1CCC(Nc2ncc3ccc(=O)n(CC(=O)O)c3n2)CC1 ; Nc1ccc(S(=O)(=O)O)cc1 ; CS(=O)(=O)N1CCC(Nc2ncc3ccc(=O)n(CC(=O)Nc4ccc(S(=O)(=O)O)cc4)c3n2)CC1 ; CS(=O)(=O)N1CCC(Nc2ncc3ccc(=O)n(CC(=O)Nc4ccc(S(=O)(=O)n5cncn5)cc4)c3n2)CC1]

### Step 1

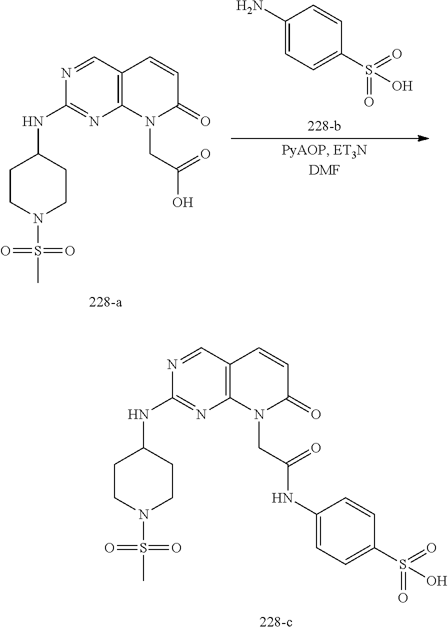 [SMILES: CS(=O)(=O)N1CCC(Nc2ncc3ccc(=O)n(CC(=O)O)c3n2)CC1 ; Nc1ccc(S(=O)(=O)O)cc1 ; CS(=O)(=O)N1CCC(Nc2ncc3ccc(=O)n(CC(=O)Nc4ccc(S(=O)(=O)O)cc4)c3n2)CC1]

To a solution of 228-a (80 mg, 0.21 mmol) and Et₃N (100 mg, 0.99 mmol) in DMF (5 mL) was added PyAOP (140 mg, 0.27 mmol) and 225-b (45 mg, 0.26 mmol) with stirring at rt. After stirred at rt for 1 h, the reaction mixture was concentrated in vacuo. The residue was purified by reverse-phase chromatography (SilaSep™ C18 silica flash cartridge, 10%-20% MeCN in H₂O with 0.1% formic acid) to afford 228-c (100 mg, 89% yield) as a yellow solid. m/z: [M+H]⁺ Calcd for C₂₁H₂₄N₆O₇S₂ 536.1; Found 537.4.

### Step 2

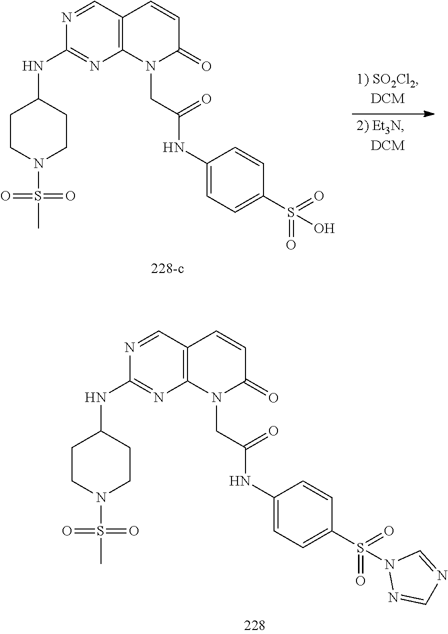 [SMILES: CS(=O)(=O)N1CCC(Nc2ncc3ccc(=O)n(CC(=O)Nc4ccc(S(=O)(=O)O)cc4)c3n2)CC1 ; CS(=O)(=O)N1CCC(Nc2ncc3ccc(=O)n(CC(=O)Nc4ccc(S(=O)(=O)n5cncn5)cc4)c3n2)CC1]

To a solution of 228-c (40 mg, 0.075 mmol) in toluene (10 mL) was added SOCl₂ (0.5 mL, 6.89 mmol) with stirring at 0° C. After stirred at 90° C. for 1 h, the reaction mixture was concentrated. Then the residue was dissolved in DCM (10 mL) and added to a solution of 1H-1,2,4-triazole (0.02 mL, 0.290 mmol) and Et₃N (100 mg, 0.99 mmol). After stirred at rt for 1 h, the reaction mixture was concentrated and purified by reverse-phase chromatography (SilaSep™ C18 silica flash cartridge, 20%-60% MeCN in H₂O with 0.1% formic acid) to afford 228 (10.5 mg, 24% yield) as a yellow solid. m/z: [M+H]⁺ Calcd for C₂₃H₂₅N₉O₆S₂ 587.1; Found 588.0. (M+H). ¹H NMR (400 MHz, DMSO) δ 11.05 (d, J=24.4 Hz, 1H), 9.35 (s, 1H), 8.63 (s, 1H), 8.31 (s, 1H), 8.06 (d, J=8.9 Hz, 2H), 7.91 (dd, J=17.7, 8.2 Hz, 3H), 7.81 (d, J=9.4 Hz, 1H), 6.33 (d, J=9.4 Hz, 1H), 5.05 (s, 2H), 3.69 (s, 1H), 3.24 (s, 2H), 2.69 (s, 3H), 2.37 (m, 2H), 1.92-1.74 (m, 2H), 1.41 (m, 2H).

### Synthesis of Compound 229

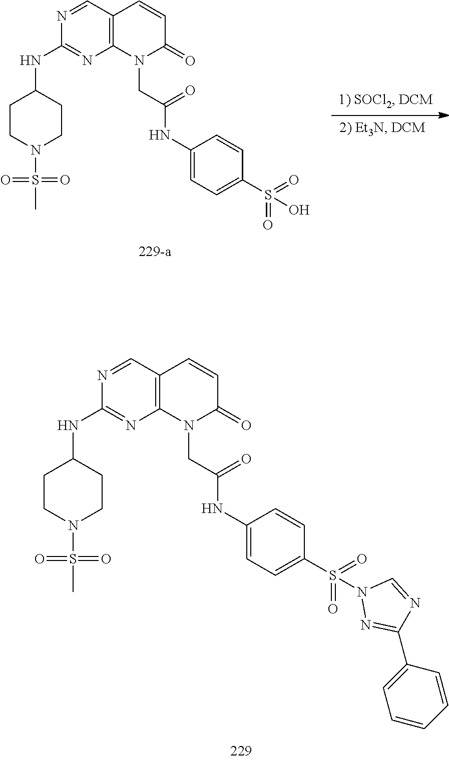

To a solution of 229-a (40 mg, 0.075 mmol) in toluene (10 mL) was added SOCl₂ (0.5 mL, 6.89 mmol) with stirring at 0° C. After stirred at 90° C. for 1 h, the reaction mixture was concentrated. Then the residue was dissolved in DCM (10 mL) and added to a solution of 3-phenyl-1H-1,2,4-triazole (20 mg, 0.14 mmol) and Et₃N (100 mg, 0.99 mmol). After stirred at rt for 1 h, the reaction mixture was concentrated and purified by reverse-phase chromatography (SilaSep™ C18 silica flash cartridge, 20%-60% MeCN in H₂O with 0.1% formic acid) to afford 229 (10.2 mg, 21% yield) as a yellow solid. m/z: [M+H]⁺ Calcd for C₂₉H₂₉N₉O₆S₂ 663.1; Found 663.7. ¹H NMR (400 MHz, DMSO) δ 11.08 (s, 1H), 9.41 (s, 1H), 8.65 (d, J=22.7 Hz, 1H), 8.12 (d, J=8.8 Hz, 2H), 8.01-7.97 (m, 2H), 7.90 (d, J=8.9 Hz, 3H), 7.80 (d, J=9.4 Hz, 1H), 7.50 (d, J=3.5 Hz, 3H), 6.33 (d, J=9.5 Hz, 1H), 5.05 (s, 2H), 3.64 (s, 1H), 3.24 (d, J=12.7 Hz, 2H), 2.66 (s, 3H), 2.40 (t, J=11.1 Hz, 2H), 1.77 (d, J=11.9 Hz, 2H), 1.38 (d, J=10.8 Hz, 2H).

### Synthesis of Compound 230

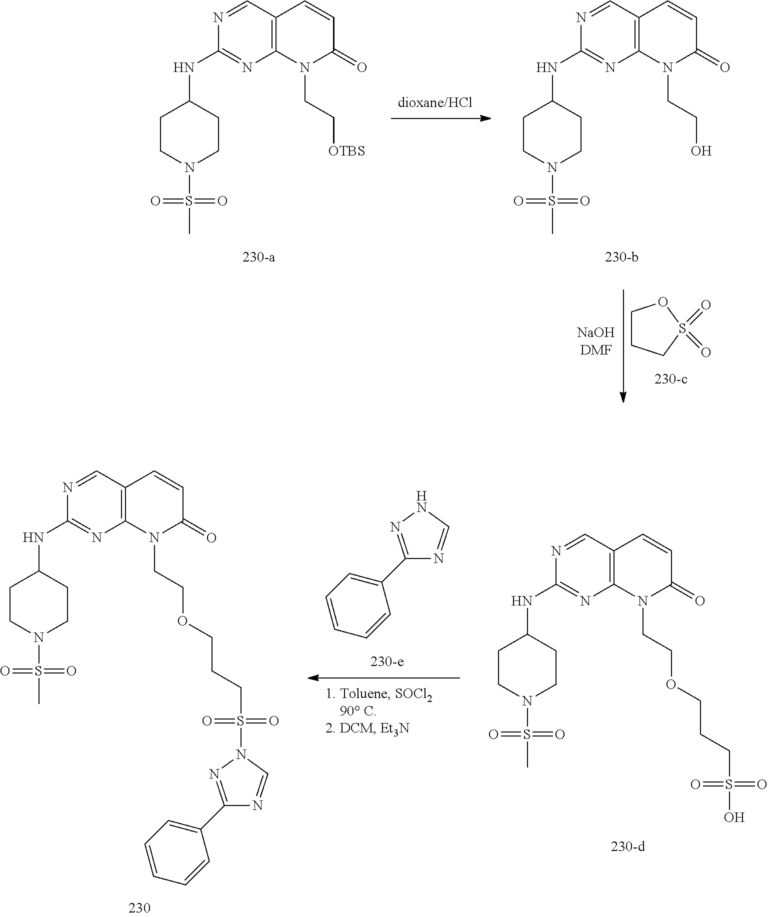 [SMILES: CC(C)(C)[Si](C)(C)OCCn1c(=O)ccc2cnc(NC3CCN(S(C)(=O)=O)CC3)nc21 ; CS(=O)(=O)N1CCC(Nc2ncc3ccc(=O)n(CCO)c3n2)CC1 ; O=S1(=O)CCCO1 ; CS(=O)(=O)N1CCC(Nc2ncc3ccc(=O)n(CCOCCCS(=O)(=O)n4cnc(-c5ccccc5)n4)c3n2)CC1 ; c1ccc(-c2nc[nH]n2)cc1 ; CS(=O)(=O)N1CCC(Nc2ncc3ccc(=O)n(CCOCCCS(=O)(=O)O)c3n2)CC1]

### Step 1

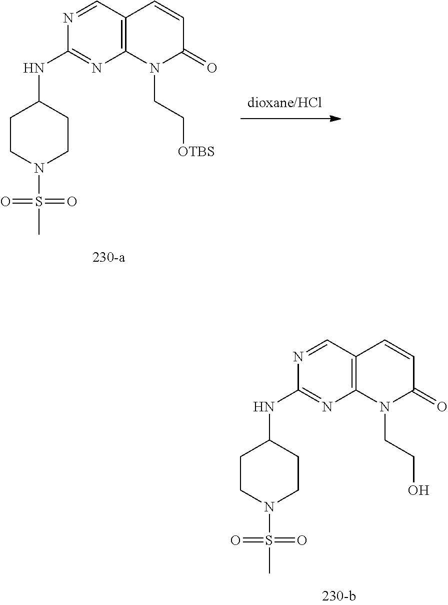 [SMILES: CC(C)(C)[Si](C)(C)OCCn1c(=O)ccc2cnc(NC3CCN(S(C)(=O)=O)CC3)nc21 ; CS(=O)(=O)N1CCC(Nc2ncc3ccc(=O)n(CCO)c3n2)CC1]

To a solution of 230-a (3 g, 6.23 mmol) in HCl/dioxane (20 mL) with stirring at rt. The solution was concentrated in vacuo to afford 230-b (2.2 g, 96% yield) as a white solid. m/z: [M+H]⁺ Calcd for C₁₅H₂₂N₅O₄S 368.1; Found 368.0.

### Step 2

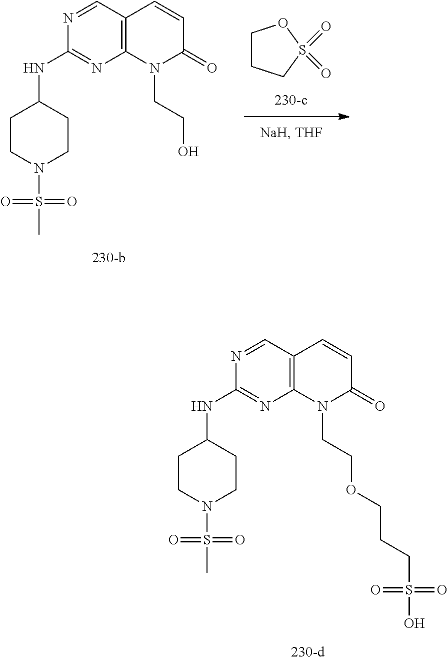

To a stirred solution of 230-b (100 mg, 0.27 mmol) and 230-c (0.02 mL, 0.25 mmol) in THF (10 mL) was added NaH (32.6 mg, 1.36 mmol) under N₂. After stirring at rt for 3 h, the solution was quenched by water and concentrated in vacuo to afford 230-d (43 mg, 32% yield) as a yellow solid. m/z: (M−H)⁻ Calcd for C₁₈H₂₆N₅O₇S₂ 488.1; Found 488.0.

### Step 3

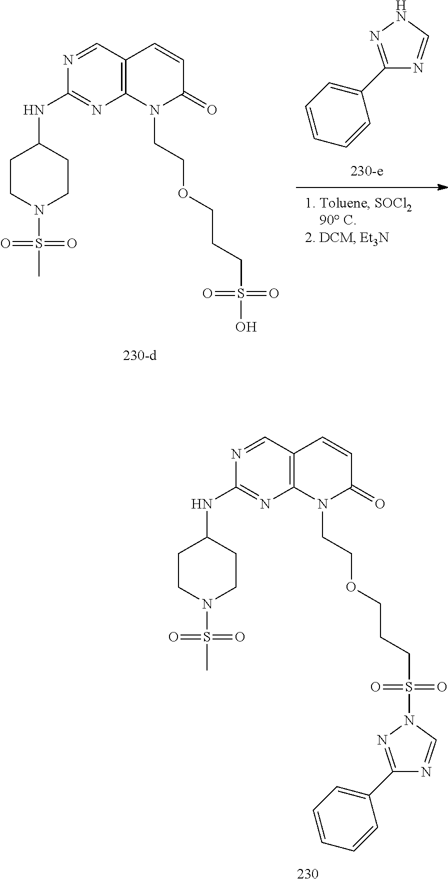 [SMILES: CS(=O)(=O)N1CCC(Nc2ncc3ccc(=O)n(CCOCCCS(=O)(=O)O)c3n2)CC1 ; c1ccc(-c2nc[nH]n2)cc1 ; CS(=O)(=O)N1CCC(Nc2ncc3ccc(=O)n(CCOCCCS(=O)(=O)n4cnc(-c5ccccc5)n4)c3n2)CC1]

To a solution of 230-d (100 mg, 0.20 mmol) in toluene (10 mL) was added SOCl₂(5 mL, 68.93 mmol) with stirring at rt. After stirred at 90° C. for 1 h, the reaction mixture was concentrated. The residue was dissolved in DCM (1 mL) and added to a solution of 230-e (0.002 mL, 0.028 mmol) and triethylamine (4 mL, 28.78 mmol) in DCM (6 mL). Then the mixture was stirred for 1 h and purified by preparative thin-layer chromatography (70% EtOAc in hexanes) and reverse-phase chromatography (SilaSep™ C18 silica flash cartridge, 20%-60% MeCN in H₂O with 0.1% formic acid) to afford 230 (8 mg, 6% yield). m/z: [M+H]⁺ Calcd for C₂₆H₃₃N₈O₆S₂ 617.2; Found 617.0. ¹H NMR (400 MHz, DMSO) δ 9.24 (s, 1H), 8.57 (s, 1H), 7.99 (dd, J=53.0, 5.4 Hz, 3H), 7.69 (d, J=9.4 Hz, 1H), 7.54 (s, 3H), 6.22 (d, J=9.3 Hz, 1H), 4.37 (d, J=6.0 Hz, 2H), 4.03-3.87 (m, 1H), 3.86-3.79 (m, 2H), 3.60-3.54 (m, 4H), 3.48 (s, 2H), 2.87 (s, 5H), 2.00 (d, J=13.5 Hz, 2H), 1.86 (s, 2H), 1.64-1.53 (m, 2H).

### Synthesis of Compound 231

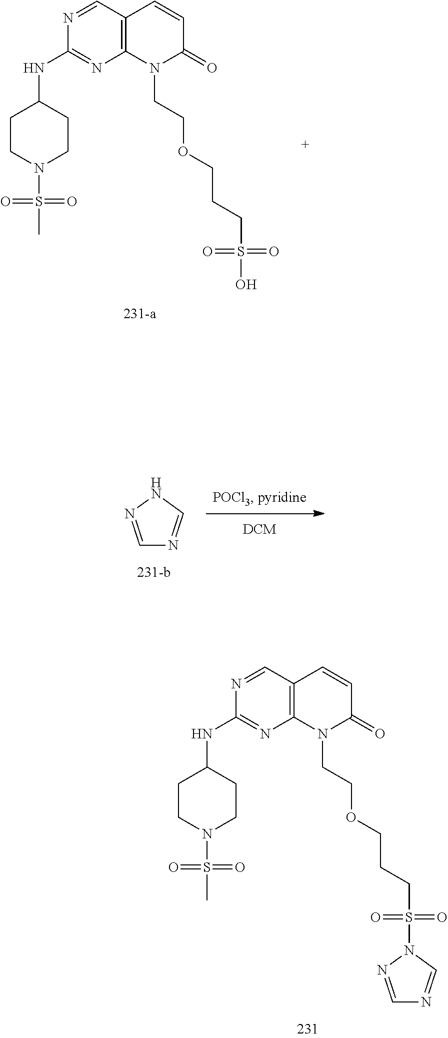 [SMILES: CS(=O)(=O)N1CCC(Nc2ncc3ccc(=O)n(CCOCCCS(=O)(=O)O)c3n2)CC1 ; c1nc[nH]n1 ; CS(=O)(=O)N1CCC(Nc2ncc3ccc(=O)n(CCOCCCS(=O)(=O)n4cncn4)c3n2)CC1]

To a solution of 231-a (50 mg, 0.10 mmol), pyridine (0.041 mL, 0.51 mmol) and 1H-1,2,4-triazole (10 mg, 0.15 mmol) in DCM (50 mL) was added POCl₃ (0.02 mL, 0.20 mmol) with stirring at 0 TC. After stirred at rt for 1 h, the reaction mixture was concentrated and purified by reverse-phase chromatography (SilaSep™ C18 silica flash cartridge, 10%-20% MeCN in H₂O with 0.1% formic acid) to afford 231 (3 mg, 5% yield) as a yellow solid. m/z: [M+H]⁺ Calcd for C₂₀H₂₈N₈O₆S₂ 540.1; Found 541.5. ¹H NMR (400 MHz, CDCl₃) δ 8.67 (s, 1H), 8.42 (s, 1H), 8.14 (s, 1H), 7.49 (d, J=9.4 Hz, 1H), 6.40 (d, J=9.4 Hz, 1H), 5.74 (d, J=190.2 Hz, 1H), 4.52 (s, 2H), 4.02 (s, 1H), 3.72 (s, 4H), 3.61-3.53 (m, 4H), 2.99 (s, 2H), 2.84 (s, 4H), 2.21 (d, J=12.8 Hz, 2H), 1.99-1.94 (m, 2H), 1.74 (s, 2H).

### Synthesis of Compound 232

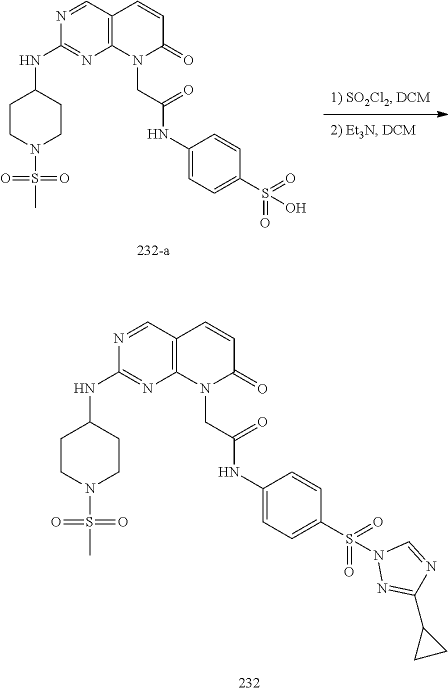 [SMILES: CS(=O)(=O)N1CCC(Nc2ncc3ccc(=O)n(CC(=O)Nc4ccc(S(=O)(=O)O)cc4)c3n2)CC1 ; CS(=O)(=O)N1CCC(Nc2ncc3ccc(=O)n(CC(=O)Nc4ccc(S(=O)(=O)n5cnc(C6CC6)n5)cc4)c3n2)CC1]

To a solution of 232-a (500 mg, 0.93 mmol) in DCM (10 mL) was added SOCl₂ (0.3 mL, 4.20 mmol) with stirring at 0° C. After stirred at 90° C. for 1 h, the reaction mixture was concentrated. Then the residue was dissolved in DCM (10 mL) and added to a solution of 3-cyclopropyl-1H-1,2,4-triazole (200 mg, 1.83 mmol) and Et₃N (150 mg, 1.49 mmol). After stirred at rt for 1 h, the reaction mixture was concentrated and purified by reverse-phase chromatography (SilaSep™ C18 silica flash cartridge, 20%-60% MeCN in H₂O with 0.1% formic acid) to afford 232 (80 mg, 14% yield) as a yellow solid. m/z: [M+H]⁺ Calcd for C₂₆H₂₉N₉O₆S₂ 627.1; Found 628.5. ¹H NMR (400 MHz, DMSO) δ 11.07 (s, 1H), 9.13 (s, 1H), 8.63 (s, 1H), 8.03 (d, J=8.6 Hz, 2H), 7.96-7.77 (m, 4H), 6.33 (d, J=9.5 Hz, 1H), 5.05 (s, 2H), 3.59 (d, J=57.7 Hz, 1H), 3.29-3.26 (m, 2H), 2.70 (s, 3H), 2.39 (t, J=10.8 Hz, 2H), 2.01 (s, 1H), 1.86 (m, 2H), 1.47 (m, 2H), 0.95 (m, 2H), 0.81 (m, 2H).

### Synthesis of Compound 233

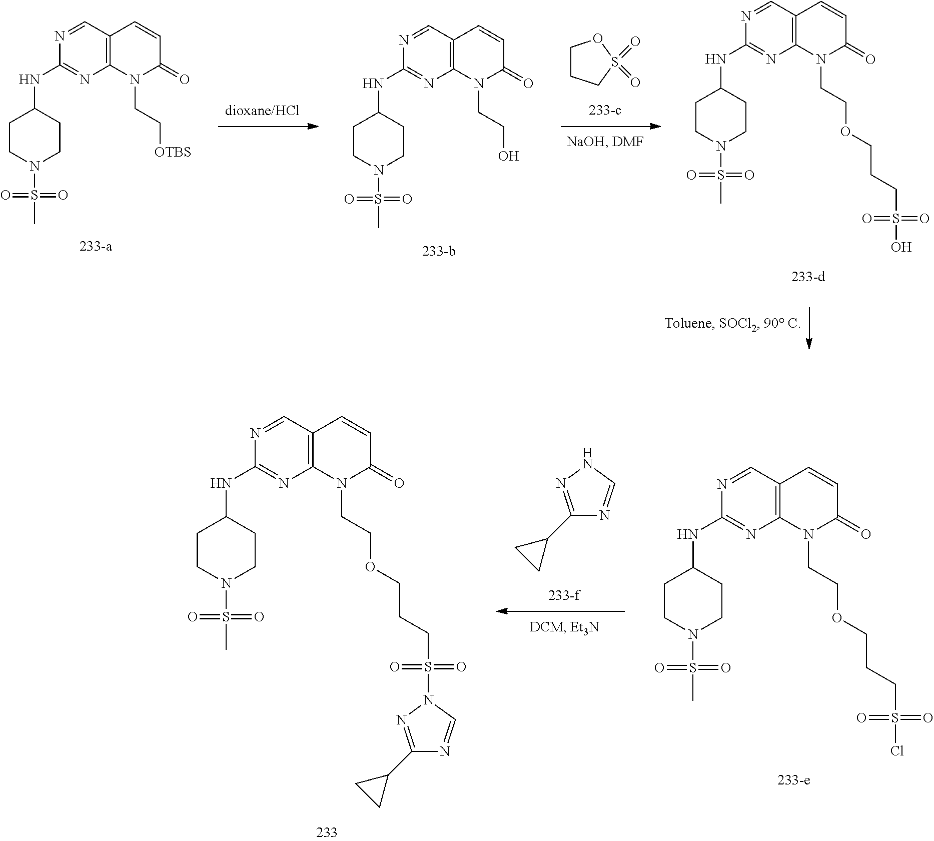

### Step 1

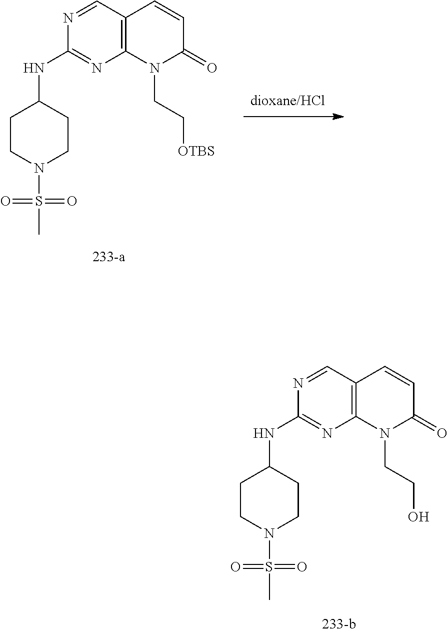 [SMILES: CC(C)(C)[Si](C)(C)OCCn1c(=O)ccc2cnc(NC3CCN(S(C)(=O)=O)CC3)nc21 ; CS(=O)(=O)N1CCC(Nc2ncc3ccc(=O)n(CCO)c3n2)CC1]

To a solution of 233-a (3 g, 6.23 mmol) in HCl/dioxane (20 mL) with stirring at rt. The solution was concentrated in vacuo to afford 233-b (2.2 g, 96% yield) as a white solid. m/z: [M+H]⁺ Calcd for C₁₅H₂₂N₅O₄S 368.1; Found 368.0.

### Step 2

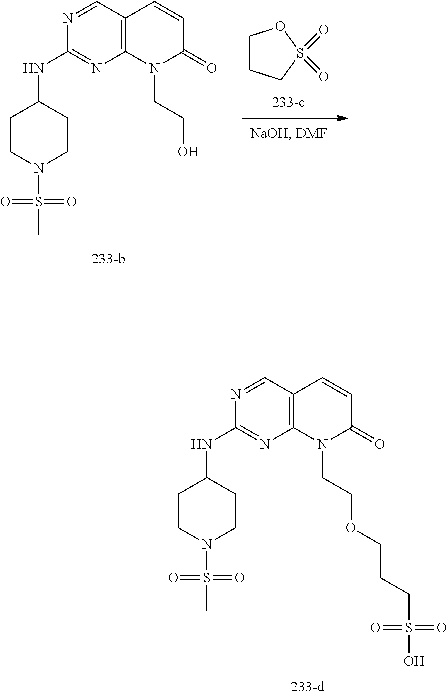

To a stirred solution of 233-b (300 mg, 0.82 mmol) and 233-c (0.07 mL, 0.83 mmol) in DMF (6 mL) was added NaOH (67 mg, 1.68 mmol) under N₂. After stirring at rt for 1 h, the solution was concentrated in vacuo and purified by reverse-phase chromatography (SilaSep™ C18 silica flash cartridge, 20%-60% MeCN in H₂O with 0.1% formic acid) to afford 233-d (80 mg, 15% yield) as a yellow solid. m/z: (M−H)⁻ Calcd for C₁₈H₂₆N₅O₇S₂ 488.1; Found 488.0.

### Step 3

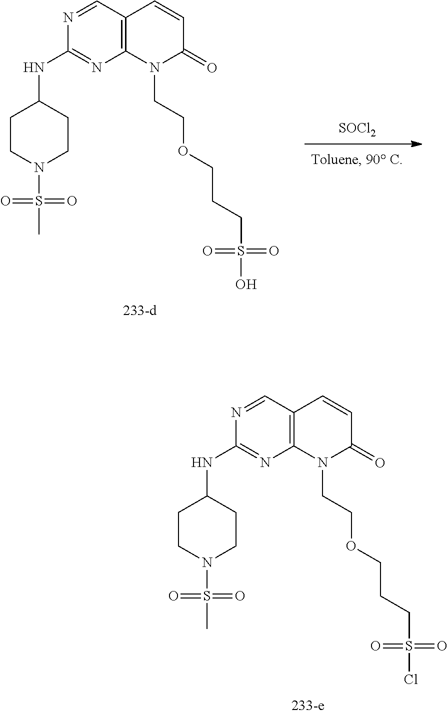 [SMILES: CS(=O)(=O)N1CCC(Nc2ncc3ccc(=O)n(CCOCCCS(=O)(=O)O)c3n2)CC1 ; "SOCl2" ; CS(=O)(=O)N1CCC(Nc2ncc3ccc(=O)n(CCOCCCS(=O)(=O)Cl)c3n2)CC1]

To a stirred solution of 233-d in toluene (10 mL) was added SOCl₂ (2 mL, 27.57 mmol) with stirring at rt. After stirred at 90° C. for 1 h, the reaction mixture was concentrated and purified by reverse-phase chromatography (SilaSep™ C18 silica flash cartridge, 20%-60% MeCN in H₂O with 0.1% formic acid) to afford 233-e (70 mg, 85% yield). m/z: [M+H]⁺ Calcd for C₁₈H₂₇ClN₅O₆S₂ 508.1; Found 508.0.

### Step 4

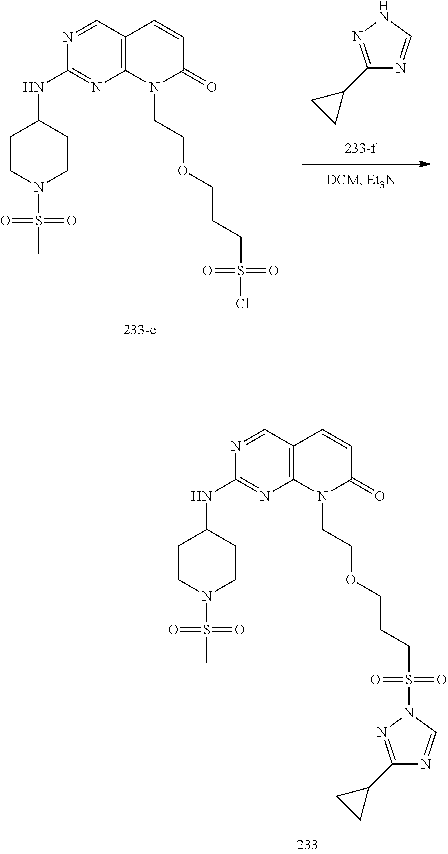 [SMILES: CS(=O)(=O)N1CCC(Nc2ncc3ccc(=O)n(CCOCCCS(=O)(=O)Cl)c3n2)CC1 ; c1nc(C2CC2)n[nH]1 ; CS(=O)(=O)N1CCC(Nc2ncc3ccc(=O)n(CCOCCCS(=O)(=O)n4cnc(C5CC5)n4)c3n2)CC1]

To a solution of 233-f in DCM (7 mL) was added triethylamine (0.08 mL, 0.58 mmol) and 233-e (100 mg, 0.2 mmol) with stirring at rt. Then the mixture was stirred for 1 h and purified by preparative thin-layer chromatography (70% EtOAc in hexanes) and reverse-phase chromatography (SilaSep™ C18 silica flash cartridge, 20%-60% MeCN in H₂O with 0.1% formic acid) to afford 233 (15 mg, 13% yield). m/z: [M+H]⁺ Calcd for C₂₃H₃₂N₈O₆S₂ 581.2; Found 581.0. ¹H NMR (400 MHz, DMSO) δ 8.96 (s, 1H), 8.61 (d, J=11.1 Hz, 1H), 8.13 (dd, J=164.8, 11.5 Hz, 1H), 7.73 (d, J=9.3 Hz, 1H), 6.25 (d, J=9.3 Hz, 1H), 4.40 (s, 2H), 3.94 (d, J=42.5 Hz, 1H), 3.69 (d, J=6.9 Hz, 2H), 3.60-3.54 (m, 6H), 2.88 (s, 5H), 2.08-1.96 (m, 3H), 1.76 (s, 2H), 1.60 (d, J=11.1 Hz, 2H), 1.00 (d, J=4.9 Hz, 2H), 0.87 (s, 2H).

### Synthesis of Compound 234

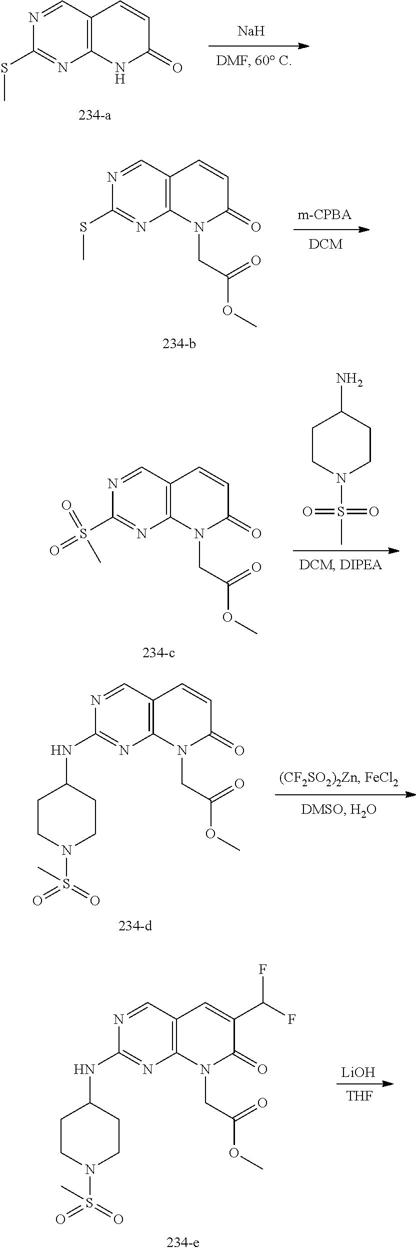

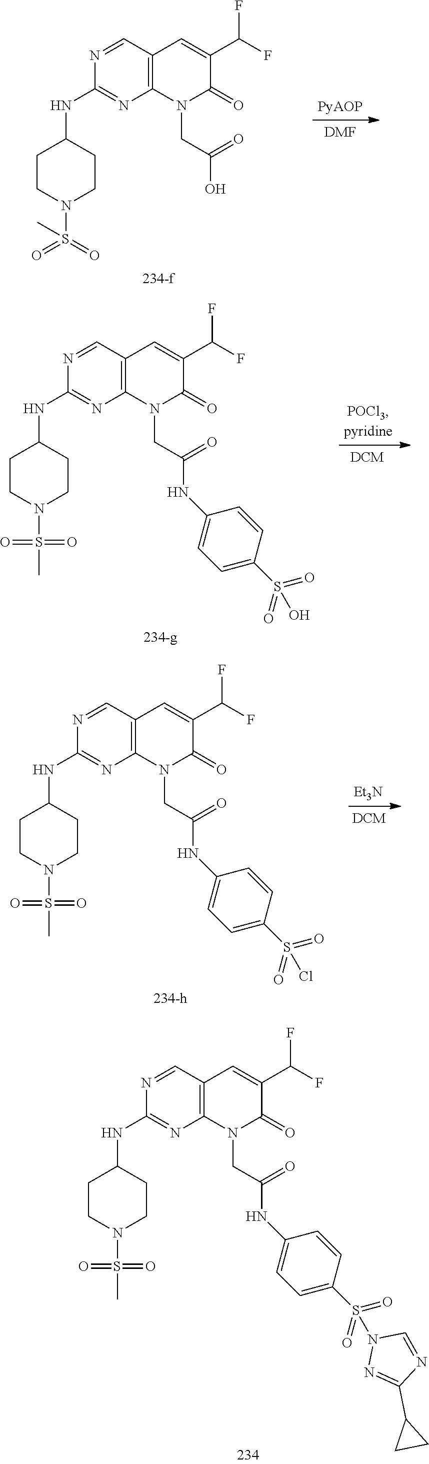

### Step 1

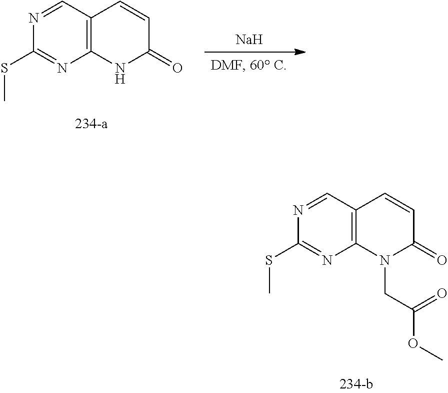

To a solution of 234-a (6 g, 31.05 mmol) in DMF (80 mL) was added NaH (60% in oil) (0.6 g, 15.00 mmol) with stirring at 0° C. After stirred at 0° C. for 30 min, methyl 2-bromoacetate (3.5 mL, 36.61 mmol) was added and stirred at 50° C. for 1 h. The reaction mixture was slowly poured into water and extracted with EtOAc (30 mL×2). The organic layer was washed with brine (30 mL), dried over anhydrous Na₂SO₄, filtered and concentrated in vacuo to afford 234-b (7.5 g, 91% yield) as a yellow solid. m/z: [M+H]⁺ Calcd for C₁₁H₁₁N₃O₃S 265.0; Found 265.8.

### Step 2

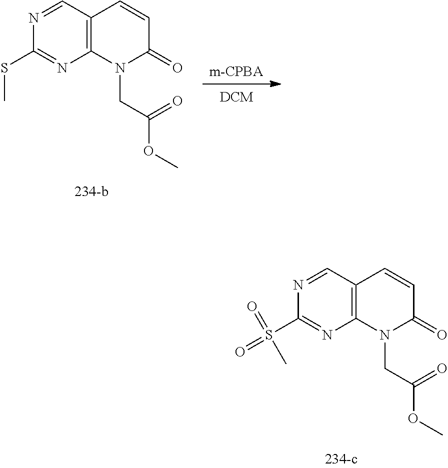

To a solution of 234-b (7.5 g, 28.27 mmol) in DCM (50 mL) was added m-CPBA (17.8 mL, 57.95 mmol) with stirring at rt. After stirred at rt for 1 h, the reaction mixture was concentrated and purified by silica gel flash chromatography (gradient 0%-50% EtOAc in hexanes) to afford 234-c (6.8 g, 81% yield) as a yellow solid. m/z: [M+H]⁺ Calcd for C₁₁H₁₁N₃O₅S 297.0; Found 297.8.

### Step 3

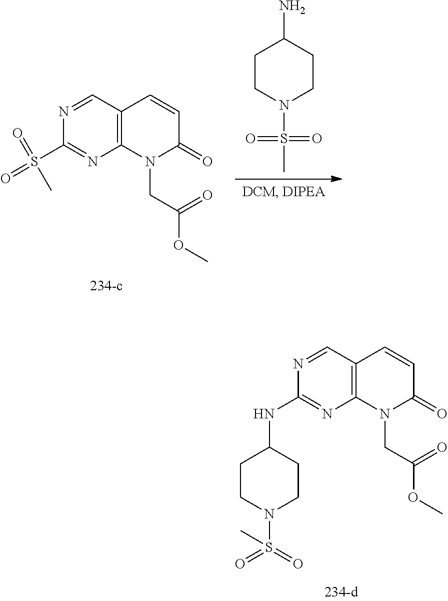

To a solution of 234-c (6.8 g, 22.87 mmol) and DIPEA (7.6 mL, 46.00 mmol) in DCM (50 mL) was added 1-methanesulfonylpiperidin-4-amine (4.5 g, 25.24 mmol) with stirring at rt. After stirred at rt for 16 h, the reaction mixture was filtered and dried to afford 234-d (5.8 g, 64% yield) as a white solid. m/z: [M+H]⁺ Calcd for C₁₆H₂₁N₅O₅S 395.1; Found 395.8.

### Step 4

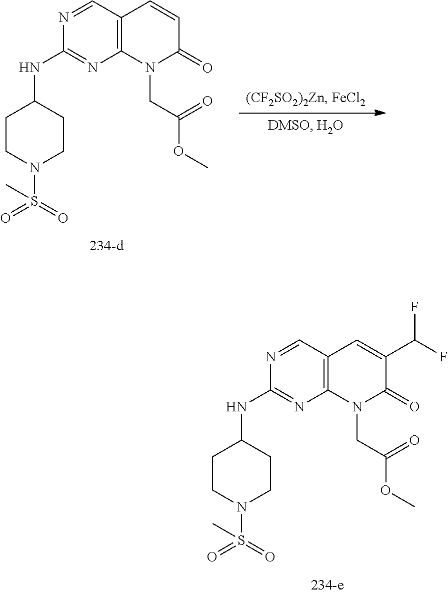

A solution of Zinc difluoromethanesulfinate (6 g, 20.30 mmol) and FeCl₂ (0.6 g, 4.73 mmol) in H₂O (10 mL) was added portion-wise to a solution of 234-d (4 g, 10.12 mmol) in DMSO (50 mL) at rt. Then, TBHP (0.97 g, 21.08 mmol) was added. After stirred at 25° C. for 18 h, a second portion of TBHP (0.47 g, 10.12 mmol) was added and stirring continued for 24 h at rt. The solution was slowly poured into water and extracted with EtOAc (30 mL×3). The organic layer was washed with brine (30 mL×2), dried over anhydrous Na₂SO₄, filtered and concentrated in vacuo. The residue was purified by silica gel flash chromatography (gradient 0%-10% MeOH in DCM) to afford 234-e (700 mg, 16% yield) as a white solid. m/z: [M+H]⁺ Calcd for C₁₇H₂₁F₂N₅O₅S 445.1; Found 446.4.

### Step 5

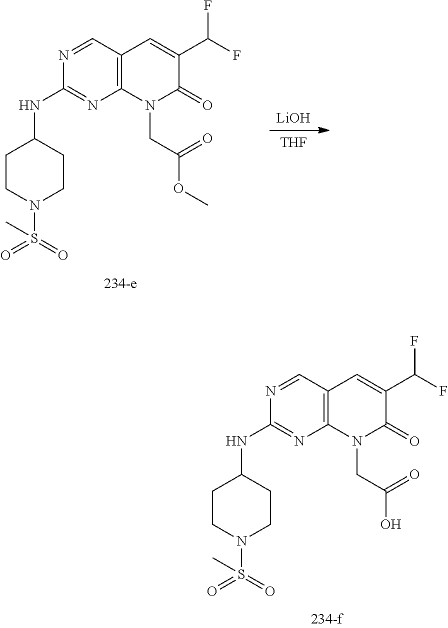 [SMILES: COC(=O)Cn1c(=O)c(C(F)F)cc2cnc(NC3CCN(S(C)(=O)=O)CC3)nc21 ; "LiOH" ; "THF" ; CS(=O)(=O)N1CCC(Nc2ncc3cc(C(F)F)c(=O)n(CC(=O)O)c3n2)CC1]

To a solution of 234-e (700 mg, 1.57 mmol) in THF (10 mL) was added aq. LiOH (130 mg, 3.10 mmol) with stirring at rt. After stirred at rt for 2 h, the reaction mixture was adjusted pH to 6 and extracted with EtOAc (30 mL×2). The organic layer was washed with brine (30 mL), dried over anhydrous Na₂SO₄, filtered and concentrated in vacuo to afford 234-f (200 mg, 30% yield) as a yellow solid. m/z: [M+H]⁺ Calcd for C₁₆H₁₉F₂N₅O₅S 431.1; Found 432.4.

### Step 6

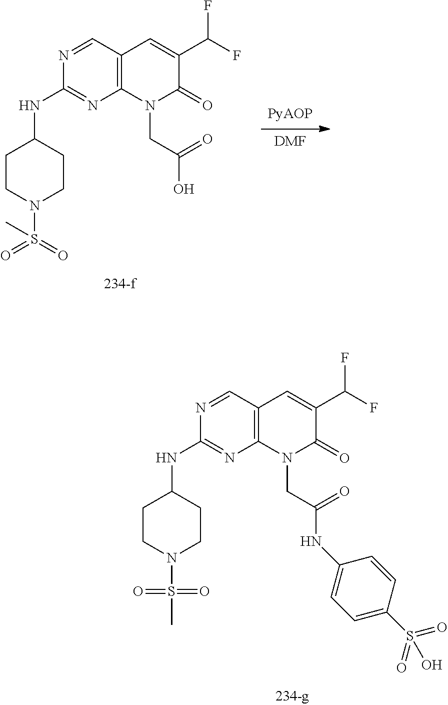

To a solution of 234-f (200 mg, 0.46 mmol), Et₃N (100 mg, 0.99 mmol) and 4-aminobenzene-1-sulfonic acid (0.05 mL, 0.46 mmol) in DMF (10 mL) was added PyAOP (300 mg, 0.58 mmol) with stirring at rt. After stirred at rt for 1 h, the reaction mixture was concentrated and purified by reverse-phase chromatography (SilaSep™ C18 silica flash cartridge, 40%-60% MeCN in H₂O with 0.1% formic acid) to afford 234-g (200 mg, 74% yield) as a yellow solid. m/z: [M+H]⁺ Calcd for C₂₂H₂₄F₂N₆O₇S₂ 586.1; Found 587.5.

### Step 7

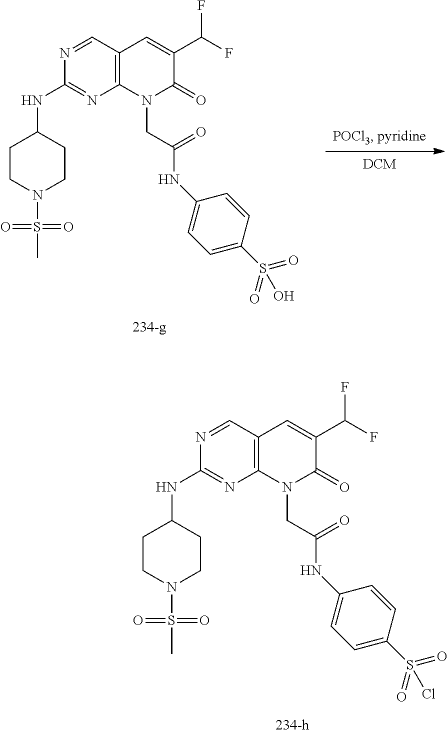

To a solution of 234-g (200 mg, 0.34 mmol) and pyridine (0.4 mL, 4.95 mmol) in DCM (10 mL) was added POCl₃ (0.24 mL, 2.61 mmol) with stirring at rt. After stirred at rt for 1 h, the reaction mixture was concentrated and purified by reverse-phase chromatography (SilaSep™ C18 silica flash cartridge, 60%-70% MeCN in H₂O with 0.1% formic acid) to afford 234-h (50 mg, 24% yield) as a yellow solid. m/z: [M+H]⁺ Calcd for C₂₂H₂₃ClF₂N₆O₆S₂ 604.1; Found 605.0.

### Step 8

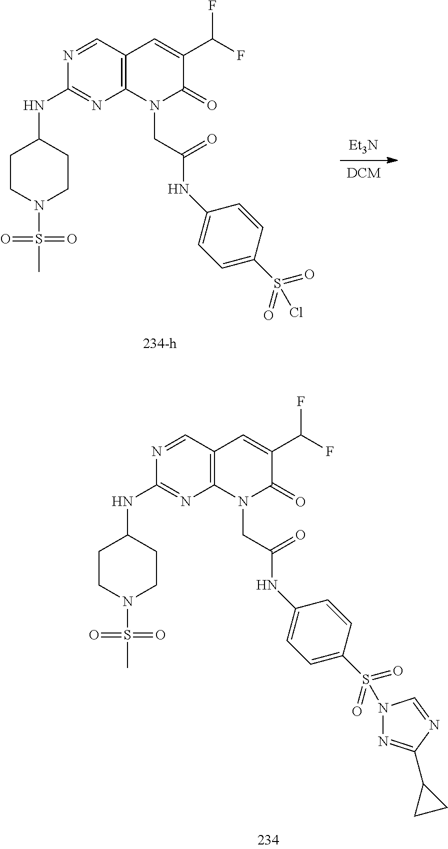

To a solution of 234-h (80 mg, 0.13 mmol) in DCM (10 mL) was added Et₃N (15 mg, 0.15 mmol) and 3-cyclopropyl-1H-1,2,4-triazole (30 mg, 0.28 mmol) with stirring at rt. After stirred at rt for 1 h, the reaction mixture was concentrated and purified by reverse-phase chromatography (SilaSep™ C18 silica flash cartridge, 40%-60% MeCN in H₂O with 0.1% formic acid) to afford 234 (15 mg, 17% yield) as a yellow solid. m/z: [M+H]⁺ Calcd for C₂₇H₂₉F₂N₉O₆S₂ 677.2; Found 678.5. ¹H NMR (400 MHz, DMSO) δ 11.10 (d, J=20.8 Hz, 1H), 9.13 (s, 1H), 8.82 (d, J=29.8 Hz, 1H), 8.23 (d, J=13.4 Hz, 2H), 8.03 (d, J=6.4 Hz, 2H), 7.89 (d, J=7.0 Hz, 2H), 6.94 (t, J=53.7 Hz, 1H), 5.08 (s, 2H), 3.81 (s, 1H), 2.70 (s, 5H), 2.38 (d, J=5.8 Hz, 2H), 2.01 (s, 1H), 1.84 (d, J=39.5 Hz, 2H), 1.47 (t, J=26.0 Hz, 2H), 0.97 (s, 2H), 0.81 (s, 2H).

### Synthesis of Compound 235

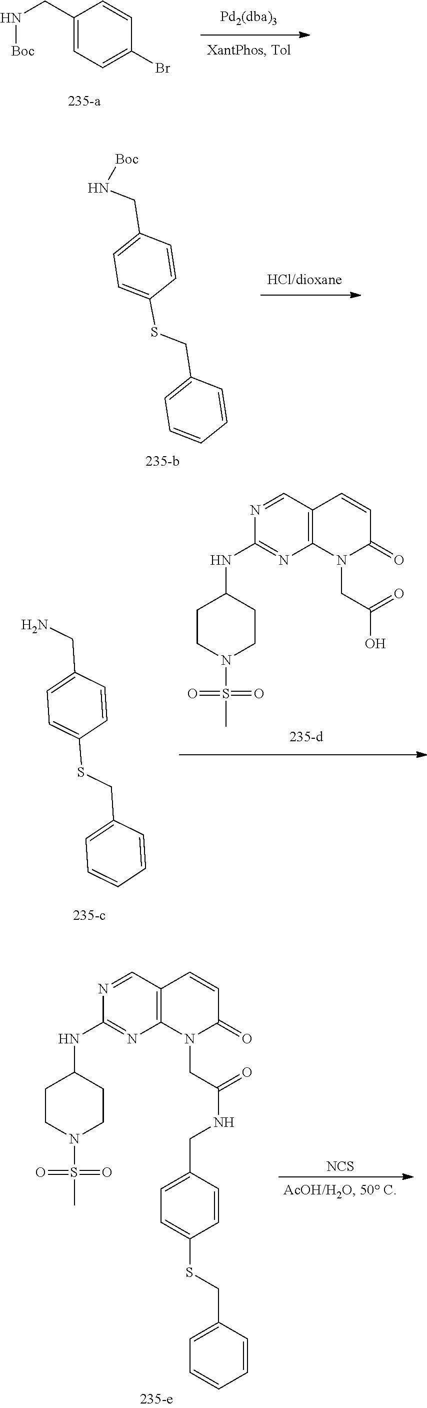 [SMILES: CC(C)(C)OC(=O)NCc1ccc(Br)cc1 ; CC(C)(C)OC(=O)NCc1ccc(SCc2ccccc2)cc1 ; NCc1ccc(SCc2ccccc2)cc1 ; CS(=O)(=O)N1CCC(Nc2ncc3ccc(=O)n(CC(=O)O)c3n2)CC1 ; CS(=O)(=O)N1CCC(Nc2ncc3ccc(=O)n(CC(=O)NCc4ccc(SCc5ccccc5)cc4)c3n2)CC1 ; "NCS"]

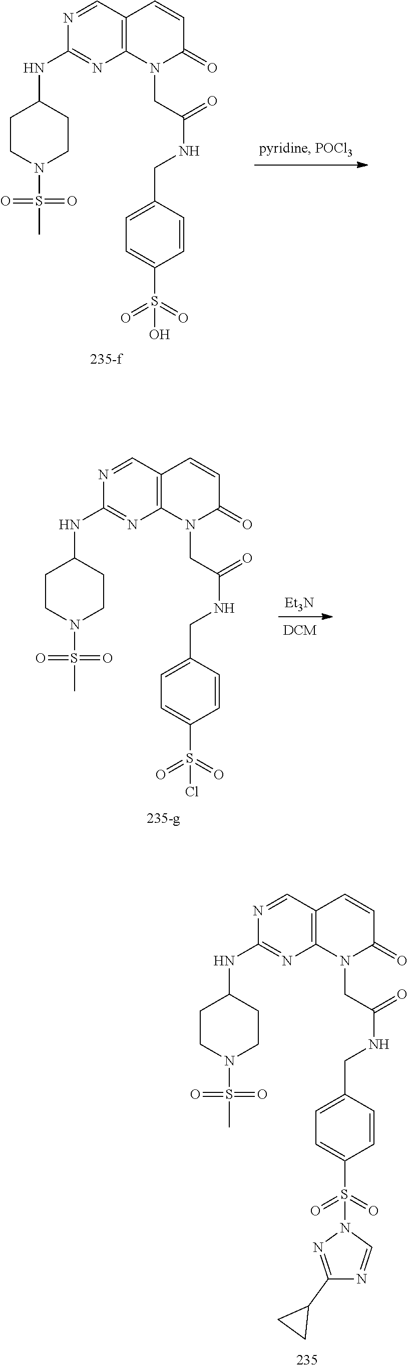 [SMILES: "Et3N" ; CS(=O)(=O)N1CCC(Nc2ncc3ccc(=O)n(CC(=O)NCc4ccc(S(=O)(=O)O)cc4)c3n2)CC1 ; CS(=O)(=O)N1CCC(Nc2ncc3ccc(=O)n(CC(=O)NCc4ccc(S(=O)(=O)Cl)cc4)c3n2)CC1 ; CS(=O)(=O)N1CCC(Nc2ncc3ccc(=O)n(CC(=O)NCc4ccc(S(=O)(=O)n5cnc(C6CC6)n5)cc4)c3n2)CC1]

### Step 1

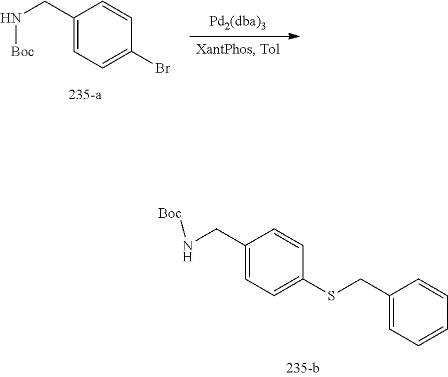 [SMILES: CC(C)(C)OC(=O)NCc1ccc(Br)cc1 ; "Pd2(dba)3" ; "XantPhos, Tol" ; CC(C)(C)OC(=O)NCc1ccc(SCc2ccccc2)cc1]

To a solution of 235-a (3 g, 10.48 mmol), phenyl methanethiol (1.3 mL, 11.27 mmol), XantPhos (0.6 g, 1.04 mmol) and Cs₂CO₃ (6.8 g, 20.87 mmol) in toluene (50 mL) was added Pd₂(dba)₃ (0.5 g, 0.55 mmol) with stirring at rt under N2. After stirred at 100° C. for 16 h, the reaction mixture was concentrated and purified by silica gel flash chromatography (gradient 0%-10% EtOAc in hexanes) to afford 235-b (2.2 g, 64% yield) as a yellow solid. m/z: [M+Na]⁺ Calcd for C₁₉H₂₃NO₂S 329.1; Found 352.5.

### Step 2

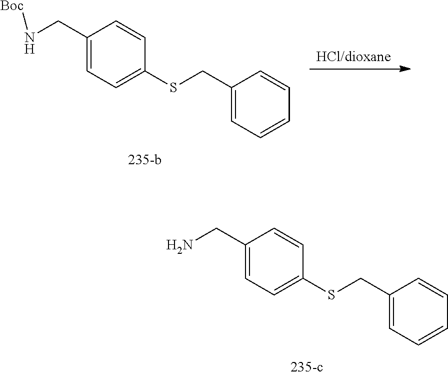 [SMILES: CC(C)(C)OC(=O)NCc1ccc(SCc2ccccc2)cc1 ; "HCl/dioxane" ; NCc1ccc(SCc2ccccc2)cc1]

The solution of 235-b (2.2 g, 6.68 mmol) in HCl/1,4-dioxane (15 mL) was stirred at rt for 1 h. Then the reaction mixture was concentrated to afford 235-c (1.5 g, 98% yield) as a yellow solid.

### Step 3

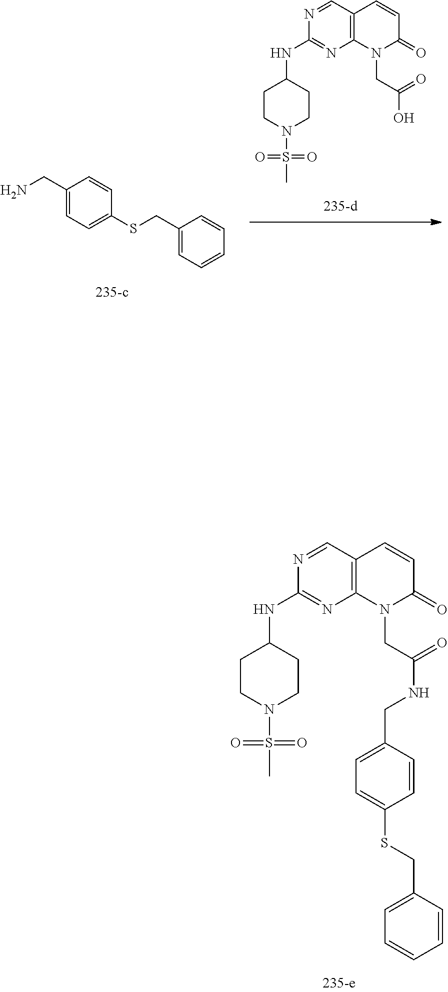 [SMILES: NCc1ccc(SCc2ccccc2)cc1 ; CS(=O)(=O)N1CCC(Nc2ncc3ccc(=O)n(CC(=O)O)c3n2)CC1 ; CS(=O)(=O)N1CCC(Nc2ncc3ccc(=O)n(CC(=O)NCc4ccc(SCc5ccccc5)cc4)c3n2)CC1]

To a solution of 235-d (0.8 g, 2.10 mmol) and HOBt (0.28 g, 2.10 mmol) in DMF (20 mL) was added 235-c (0.6 g, 2.62 mmol) and EDCI (0.40 g, 2.10 mmol) with stirring at rt. After stirred at rt for 16 h, the reaction mixture was poured into water and extracted with EtOAc (50 mL×2). The organic layer was washed with brine (30 mL), dried over anhydrous Na₂SO₄, filtered and concentrated in vacuo. The residue was purified by silica gel flash chromatography (gradient 0%-10% MeOH in DCM) to afford 235-e (1 g, 80% yield) as a brown solid. m/z: [M+H]⁺ Calcd for C₂₉H₃₂N₆O₄S₂ 592.2; Found 593.5.

### Step 4

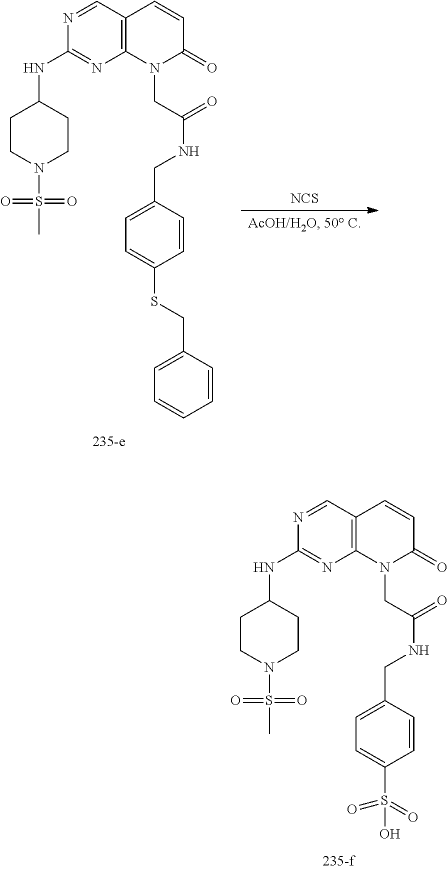 [SMILES: CS(=O)(=O)N1CCC(Nc2ncc3ccc(=O)n(CC(=O)NCc4ccc(SCc5ccccc5)cc4)c3n2)CC1 ; "AcOH/H2O, 50° C." ; "NCS" ; CS(=O)(=O)N1CCC(Nc2ncc3ccc(=O)n(CC(=O)NCc4ccc(S(=O)(=O)O)cc4)c3n2)CC1]

To a solution of 235-e (1 g, 1.69 mmol) in AcOH (10 mL) and H₂O (10 mL) was added NCS (0.46 g, 3.45 mmol) with stirring at 0° C. After stirred at 50° C. for 3 h, the reaction mixture was poured into water, filtered and dried to afford 235-f (450 mg, 48% yield) as a yellow solid. m/z: [M+H]⁺ Calcd for C₂₂H₂₆N₆O₇S₂ 550.1; Found 551.1.

### Step 5

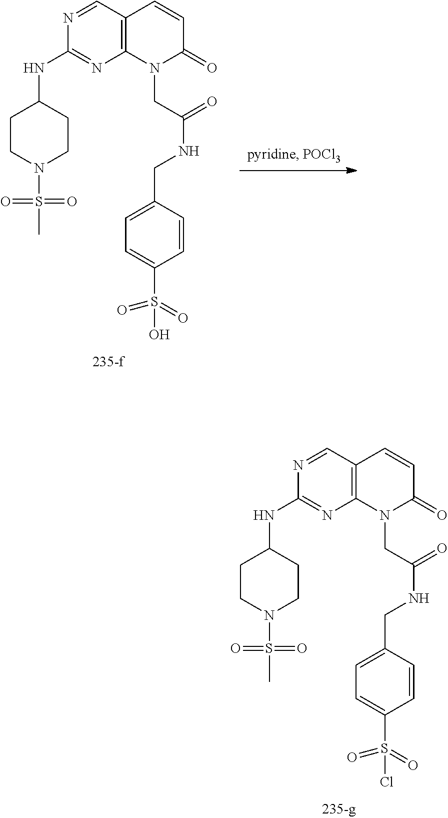 [SMILES: CS(=O)(=O)N1CCC(Nc2ncc3ccc(=O)n(CC(=O)NCc4ccc(S(=O)(=O)O)cc4)c3n2)CC1 ; "pyridine, POCl3" ; CS(=O)(=O)N1CCC(Nc2ncc3ccc(=O)n(CC(=O)NCc4ccc(S(=O)(=O)Cl)cc4)c3n2)CC1]

To a solution of 235-f (400 mg, 0.73 mmol) and pyridine (300 mg, 3.79 mmol) in DCM (10 mL) was added POCl₃ (400 mg, 2.61 mmol) with stirring at rt. After stirred at rt for 1 h, the reaction mixture was concentrated and purified by reverse-phase chromatography (SilaSep™ C18 silica flash cartridge, 60%-70% MeCN in H₂O with 0.1% formic acid) to afford 235-g (50 mg, 12% yield) as a yellow solid. m/z: [M+H]⁺ Calcd for C₂₂H₂₅ClN₆O₆S₂ 568.1; Found 569.1.

### Step 6

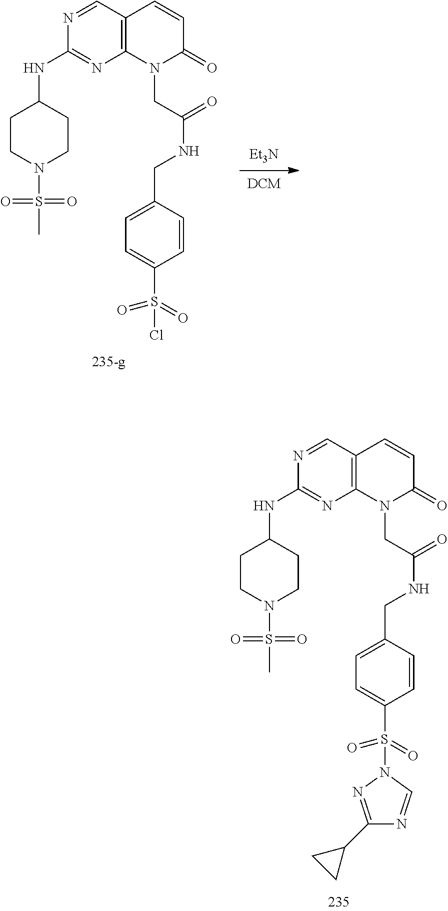

To a solution of 235-g (20 mg, 0.035 mmol) in DCM (50 mL) was added Et₃N (10 mg, 0.099 mmol) and 3-cyclopropyl-1H-1,2,4-triazole (10 mg, 0.092 mmol) with stirring at rt. After stirred at rt for 1 h, the reaction mixture was concentrated and purified by reverse-phase chromatography (SilaSep™ C18 silica flash cartridge, 40%-60% MeCN in H₂O with 0.1% formic acid) to afford 235 (5.9 mg, 26% yield) as a white solid. m/z: [M+H]⁺ Calcd for C₂₇H₃₁N₉O₆S₂ 641.2; Found 642.1. ¹H NMR (400 MHz, DMSO) δ 9.17 (s, 1H), 8.82-8.55 (m, 2H), 8.06-7.96 (m, 2H), 7.93 (d, J=6.6 Hz, 1H), 7.78 (d, J=9.4 Hz, 1H), 7.58 (d, J=8.0 Hz, 2H), 6.31 (d, J=9.4 Hz, 1H), 4.90 (s, 2H), 4.39 (s, 2H), 4.03 (s, 1H), 3.54 (s, 2H), 2.83 (m, 5H), 1.95 (m, 3H), 1.57 (m, 2H), 0.93 (m, 4H).

### Synthesis of Compound 236

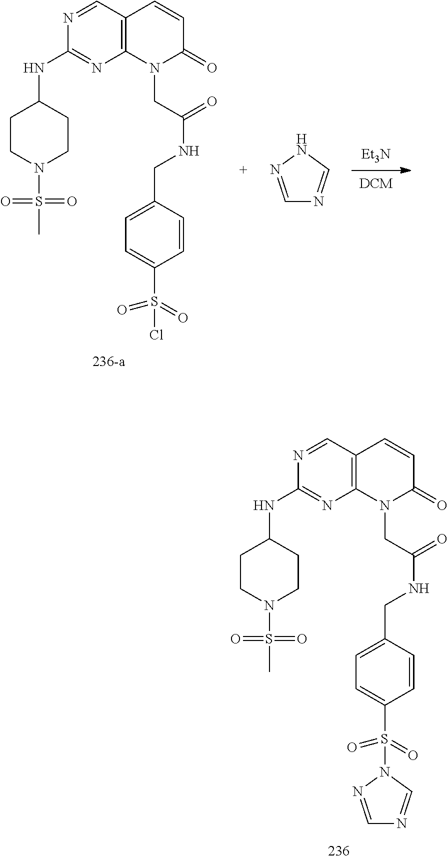

To a solution of 236-a (20 mg, 0.035 mmol) in DCM (10 mL) was added Et₃N (10 mg, 0.099 mmol) and 1H-1,2,4-triazole (0.009 mL, 0.15 mmol) with stirring at rt. After stirred at rt for 1 h, the reaction mixture was concentrated and purified by reverse-phase chromatography (SilaSep™ C18 silica flash cartridge, 40%-60% MeCN in H₂O with 0.1% formic acid) to afford 236 (1.2 mg, 6% yield) as a yellow solid. m/z: [M+H]⁺ Calcd for C₂₄H₂₇N₉O₆S₂ 601.1; Found 602.5. ¹H NMR (400 MHz, CD₃CN) δ 8.93 (s, 1H), 8.52 (s, 1H), 8.10 (s, 1H), 8.02 (d, J=8.4 Hz, 2H), 7.69 (d, J=9.6 Hz, 1H), 7.54 (d, J=7.1 Hz, 2H), 7.18 (s, 1H), 6.34 (d, J=9.4 Hz, 1H), 6.25 (s, 1H), 4.97 (s, 2H), 4.46 (s, 2H), 3.82 (s, 1H), 3.62 (s, 2H), 2.81 (s, 5H), 1.59 (s, 2H), 1.30 (s, 2H).

### Synthesis of Compound 237

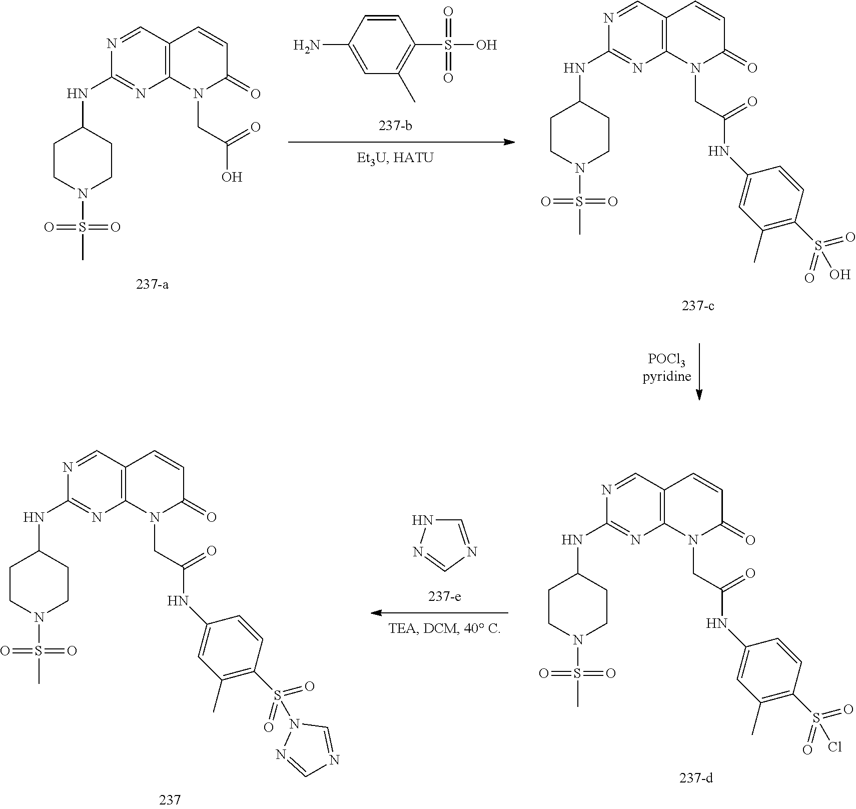 [SMILES: CS(=O)(=O)N1CCC(Nc2ncc3ccc(=O)n(CC(=O)O)c3n2)CC1 ; Cc1cc(N)ccc1S(=O)(=O)O ; Cc1cc(NC(=O)Cn2c(=O)ccc3cnc(NC4CCN(S(C)(=O)=O)CC4)nc32)ccc1S(=O)(=O)O ; Cc1cc(NC(=O)Cn2c(=O)ccc3cnc(NC4CCN(S(C)(=O)=O)CC4)nc32)ccc1S(=O)(=O)n1cncn1 ; c1nc[nH]n1 ; Cc1cc(NC(=O)Cn2c(=O)ccc3cnc(NC4CCN(S(C)(=O)=O)CC4)nc32)ccc1S(=O)(=O)Cl]

### Step 1

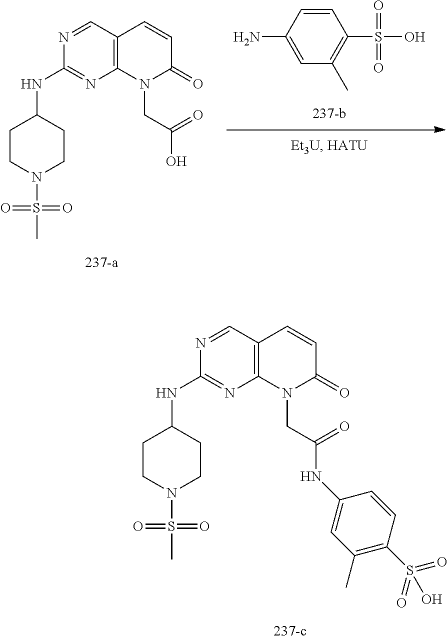 [SMILES: CS(=O)(=O)N1CCC(Nc2ncc3ccc(=O)n(CC(=O)O)c3n2)CC1 ; Cc1cc(N)ccc1S(=O)(=O)O ; Cc1cc(NC(=O)Cn2c(=O)ccc3cnc(NC4CCN(S(C)(=O)=O)CC4)nc32)ccc1S(=O)(=O)O]

To a solution of 237-a (500 mg, 1.31 mmol), Et₃N (280 mg, 2.77 mmol) and HATU (630 mg, 1.66 mmol) in DMF (50 mL) was added 237-b (0.2 mL, 1.60 mmol) with stirring at rt. After stirred at rt for 1 h, the reaction mixture was concentrated and purified by reverse-phase chromatography (SilaSep™ C18 silica flash cartridge, 40%-60% MeOH in H₂O with 0.1% formic acid) to afford 237-c (640 mg, 89% yield) as a yellow solid. m/z: [M+H]⁺ Calcd for C₂₂H₂₇N₆O₇S₂ 569.1; Found 551.1.

### Step 2

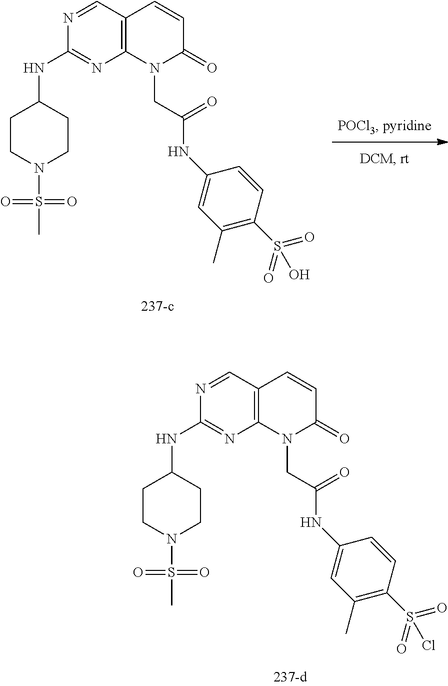 [SMILES: Cc1cc(NC(=O)Cn2c(=O)ccc3cnc(NC4CCN(S(C)(=O)=O)CC4)nc32)ccc1S(=O)(=O)O ; Cc1cc(NC(=O)Cn2c(=O)ccc3cnc(NC4CCN(S(C)(=O)=O)CC4)nc32)ccc1S(=O)(=O)Cl]

To a solution of 237-c (470 mg, 0.85 mmol) in dry DCM (20 mL) was added pyridine (0.35 mL, 4.27 mmol) and POCl₃ (0.6 mL, 5.98 mmol). The reaction was stirred at room temperature for 1 h. Ice-water was added thereto, the mixture was extracted with DCM (30 ml×2) and dried over anhydrous Na₂SO₄, filtered and concentrated in vacuo. The residue was dried to afford 237-d (70 mg, 14% yield). m/z: [M+H]⁺ Calcd for C₂₂H₂₆ClN₆O₆S₂ 569.1; Found 569.1.

### Step 3

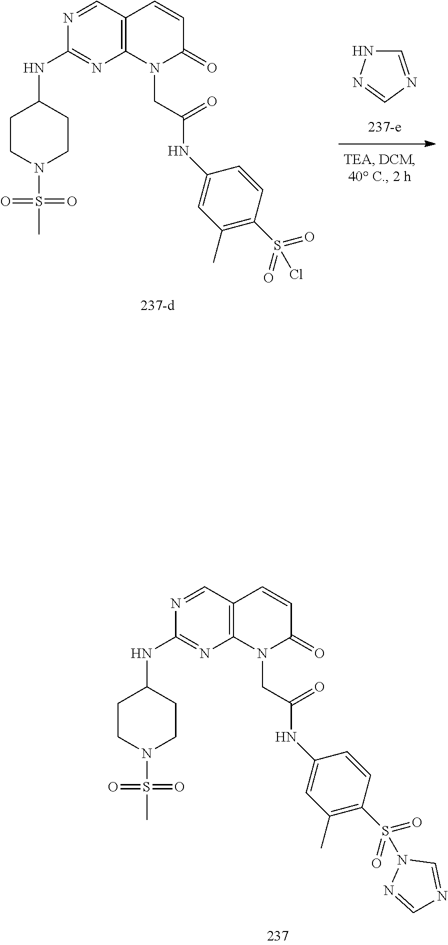 [SMILES: Cc1cc(NC(=O)Cn2c(=O)ccc3cnc(NC4CCN(S(C)(=O)=O)CC4)nc32)ccc1S(=O)(=O)Cl ; c1nc[nH]n1 ; Cc1cc(NC(=O)Cn2c(=O)ccc3cnc(NC4CCN(S(C)(=O)=O)CC4)nc32)ccc1S(=O)(=O)n1cncn1]

To a solution of 237-d (50 mg, 0.088 mmol) and TEA (0.01 mL, 0.088 mmol) in dry DCM (5 mL) was added 237-e (0.01 mL, 0.15 mmol). After stirred at 40 for 2 h, the reaction was concentrated in vacuo. The residue was purified by reverse phase chromatography (SilaSep™ C18 silica flash cartridge, 40%-60% MeCN in H₂O) to afford 237 (6.8 mg, 13% yield) as a white solid. m/z: [M+H]⁺ Calcd for C₂₄H₂₆N₉O₆S₂ 602.1; Found 602.1. ¹H NMR (400 MHz, CD₃CN) δ 9.16 (s, 1H), 8.96 (s, 1H), 8.15 (s, 3H), 8.09 (s, 1H), 7.71 (d, J=9.4 Hz, 2H), 6.37 (d, J=10.1 Hz, 1H), 5.09 (s, 2H), 3.81 (s, 1H), 3.40 (s, 2H), 2.65 (s, 3H), 2.53 (s, 3H), 2.45 (s, 2H), 1.80 (s, 2H), 1.49 (s, 2H).

### Synthesis of Compound 238

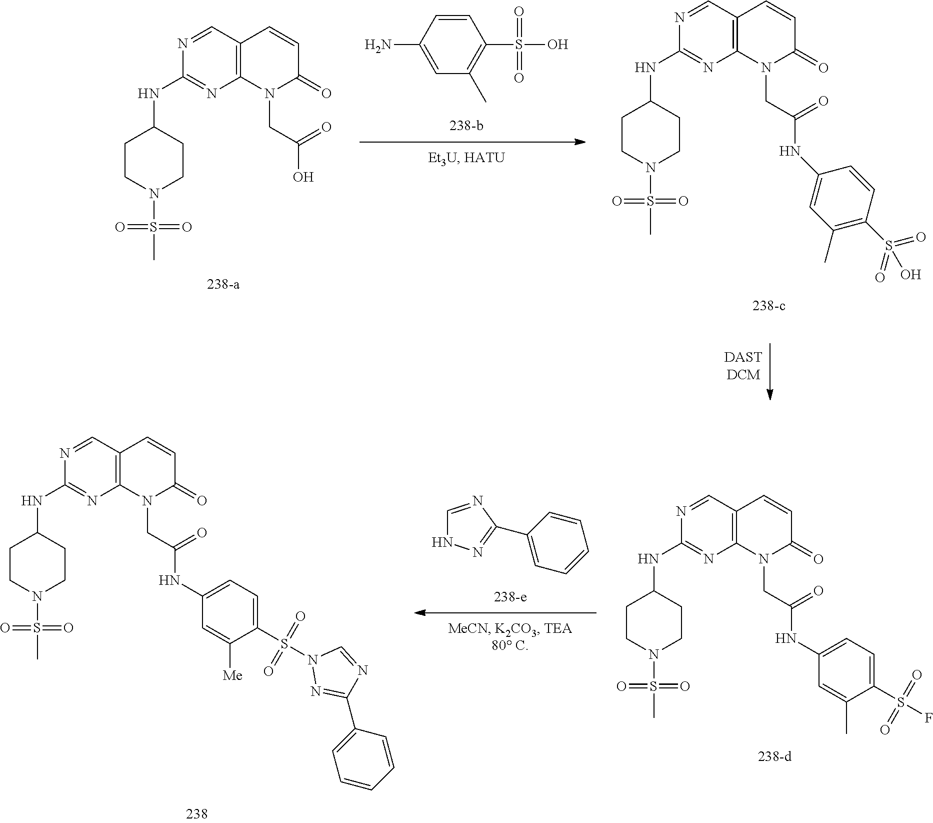 [SMILES: CS(=O)(=O)N1CCC(Nc2ncc3ccc(=O)n(CC(=O)O)c3n2)CC1 ; Cc1cc(N)ccc1S(=O)(=O)O ; Cc1cc(NC(=O)Cn2c(=O)ccc3cnc(NC4CCN(S(C)(=O)=O)CC4)nc32)ccc1S(=O)(=O)O ; Cc1cc(NC(=O)Cn2c(=O)ccc3cnc(NC4CCN(S(C)(=O)=O)CC4)nc32)ccc1S(=O)(=O)n1cnc(-c2ccccc2)n1 ; c1ccc(-c2nc[nH]n2)cc1 ; Cc1cc(NC(=O)Cn2c(=O)ccc3cnc(NC4CCN(S(C)(=O)=O)CC4)nc32)ccc1S(=O)(=O)F]

### Step 1

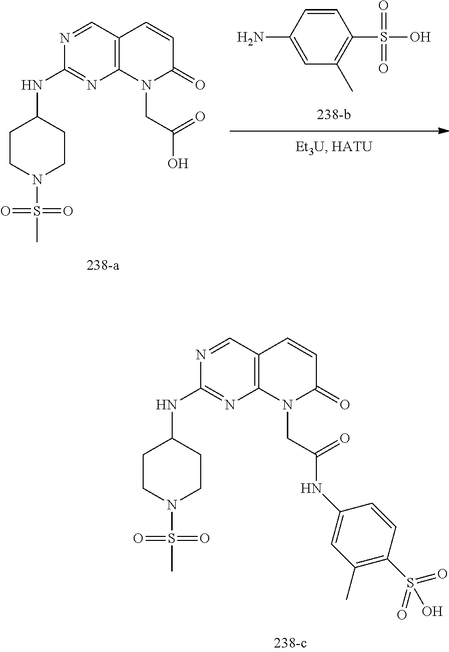 [SMILES: CS(=O)(=O)N1CCC(Nc2ncc3ccc(=O)n(CC(=O)O)c3n2)CC1 ; Cc1cc(N)ccc1S(=O)(=O)O ; Cc1cc(NC(=O)Cn2c(=O)ccc3cnc(NC4CCN(S(C)(=O)=O)CC4)nc32)ccc1S(=O)(=O)O]

To a solution of 238-a (500 mg, 1.31 mmol), Et₃N (280 mg, 2.77 mmol) and HATU (630 mg, 1.66 mmol) in DMF (50 mL) was added 238-b (0.2 mL, 1.60 mmol) with stirring at rt. After stirred at rt for 1 h, the reaction mixture was concentrated and purified by reverse-phase chromatography (SilaSep™ C18 silica flash cartridge, 40%-60% MeOH in H₂O with 0.1% formic acid) to afford 238-c (640 mg, 89% yield) as a yellow solid. m/z: [M+H]⁺ Calcd for C₂₂H₂₇N₆O₇S₂ 551.1; Found 551.1.

### Step 2

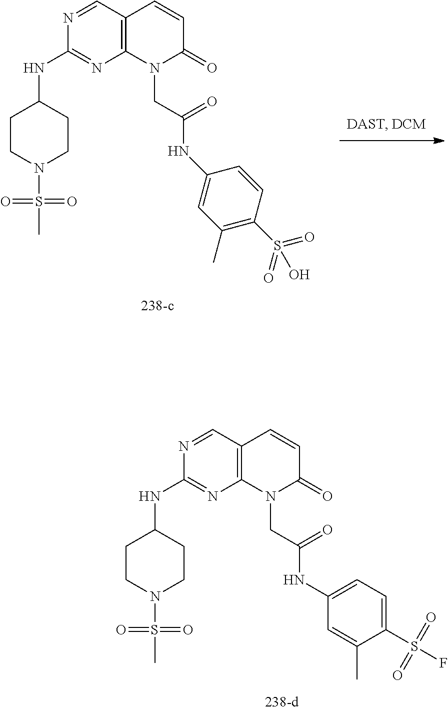 [SMILES: Cc1cc(NC(=O)Cn2c(=O)ccc3cnc(NC4CCN(S(C)(=O)=O)CC4)nc32)ccc1S(=O)(=O)O ; Cc1cc(NC(=O)Cn2c(=O)ccc3cnc(NC4CCN(S(C)(=O)=O)CC4)nc32)ccc1S(=O)(=O)F]

To a solution of 238-c (200 mg, 0.36 mmol) in dry DCM (30 mL) was added DAST (0.2 mL, 1.82 mmol) at 0° C. The reaction was stirred at room temperature for 2 h. Water was added thereto, the mixture was extracted with DCM (15 mL×2). The organic layer was extracted with THF (20 mL), and dried over anhydrous Na₂SO₄ filtered and concentrated in vacuo. The residue was purified by silica gel flash chromatography (gradient 0%-10% MeOH in DCM) to afford 238-d (80 mg, 40% yield) as a white solid. m/z: [M+H]⁺ Calcd for C₂₂H₂₆FN₆O₆S₂ 553.1; Found 553.0.

### Step 3

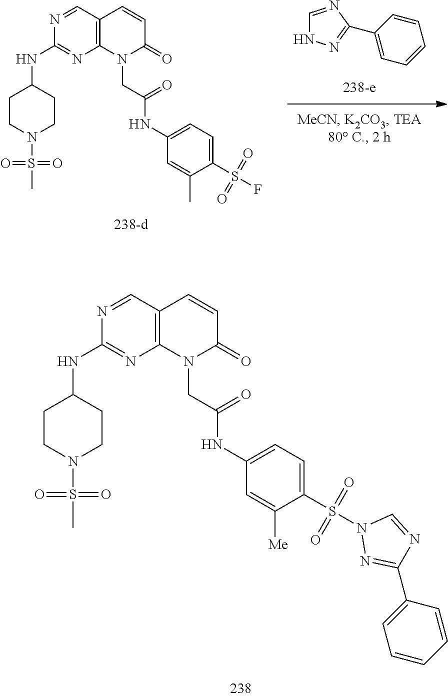 [SMILES: Cc1cc(NC(=O)Cn2c(=O)ccc3cnc(NC4CCN(S(C)(=O)=O)CC4)nc32)ccc1S(=O)(=O)F ; c1ccc(-c2nc[nH]n2)cc1 ; Cc1cc(NC(=O)Cn2c(=O)ccc3cnc(NC4CCN(S(C)(=O)=O)CC4)nc32)ccc1S(=O)(=O)n1cnc(-c2ccccc2)n1]

To a solution of 238-d (80 mg, 0.145 mmmol) in MeCN (6 mL) was added 238-e and K2CO₃ (20 mg, 0.15 mmol). After 2 h at 80° C., the reaction was concentrated in vacuo. The residue was purified by reverse-phase chromatography (SilaSep™ C18 silica flash cartridge, 40%-60% MeCN in H₂O) to afford 238 (10 mg, 10% yield) as a white solid. m/z: [M+H]⁺ Calcd for C₃₀H₃₂N₉O₆S₂ 678.2; Found 678.0. ¹H NMR (400 MHz, DMSO) δ 10.98 (d, J=18.2 Hz, 1H), 9.50 (s, 1H), 8.65 (d, J=22.2 Hz, 1H), 8.17 (d, J=9.0 Hz, 1H), 7.95 (dd, J=20.9, 5.3 Hz, 3H), 7.79 (t, J=10.3 Hz, 2H), 7.69 (s, 1H), 7.50 (s, 3H), 6.33 (d, J=9.1 Hz, 1H), 5.05 (s, 2H), 3.68 (s, 1H), 3.51 (s, 2H), 2.77 (d, J=65.3 Hz, 3H), 2.54 (s, 3H), 1.84 (d, J=46.8 Hz, 2H), 1.40 (d, J=9.0 Hz, 2H).

### Synthesis of Compound 239

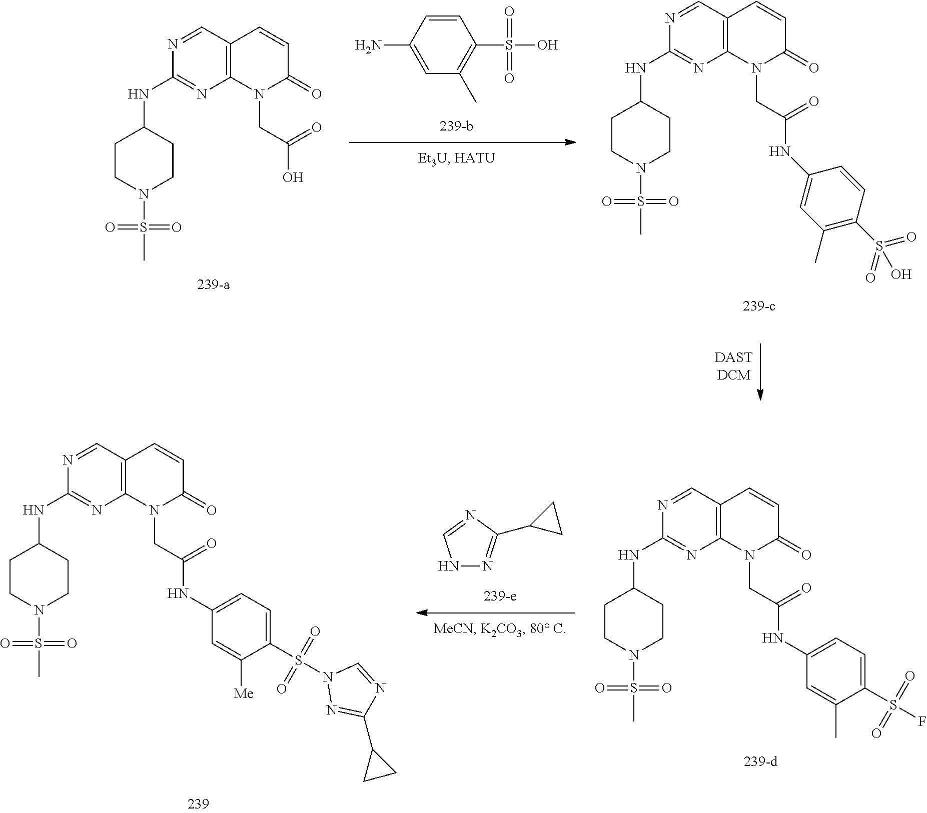 [SMILES: CS(=O)(=O)N1CCC(Nc2ncc3ccc(=O)n(CC(=O)O)c3n2)CC1 ; Cc1cc(N)ccc1S(=O)(=O)O ; Cc1cc(NC(=O)Cn2c(=O)ccc3cnc(NC4CCN(S(C)(=O)=O)CC4)nc32)ccc1S(=O)(=O)O ; Cc1cc(NC(=O)Cn2c(=O)ccc3cnc(NC4CCN(S(C)(=O)=O)CC4)nc32)ccc1S(=O)(=O)n1cnc(C2CC2)n1 ; c1nc(C2CC2)n[nH]1 ; Cc1cc(NC(=O)Cn2c(=O)ccc3cnc(NC4CCN(S(C)(=O)=O)CC4)nc32)ccc1S(=O)(=O)F]

### Step 1

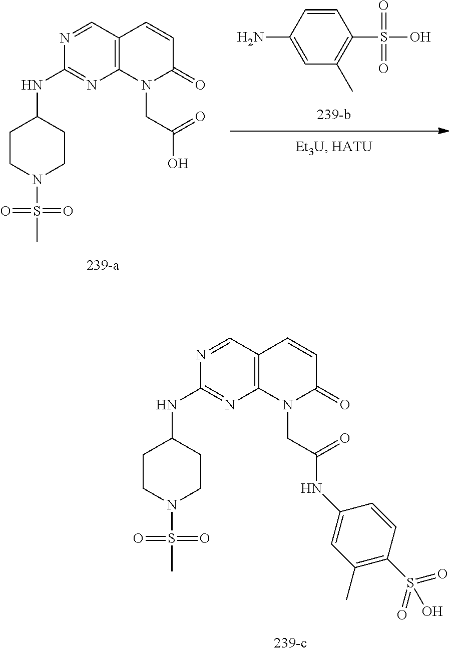 [SMILES: CS(=O)(=O)N1CCC(Nc2ncc3ccc(=O)n(CC(=O)O)c3n2)CC1 ; Cc1cc(N)ccc1S(=O)(=O)O ; Cc1cc(NC(=O)Cn2c(=O)ccc3cnc(NC4CCN(S(C)(=O)=O)CC4)nc32)ccc1S(=O)(=O)O]

To a solution of 239-a (500 mg, 1.31 mmol), Et₃N (280 mg, 2.77 mmol) and HATU (630 mg, 1.66 mmol) in DMF (50 mL) was added 239-b (0.2 mL, 1.60 mmol) with stirring at rt. After stirred at rt for 1 h, the reaction mixture was concentrated and purified by reverse-phase chromatography (SilaSep™ C18 silica flash cartridge, 40%-60% MeOH in H₂O with 0.1% formic acid) to afford 239-c (640 mg, 89% yield) as a yellow solid. m/z: [M+H]⁺ Calcd for C₂₂H₂₇N₆O₇S₂ 551.1; Found 551.1.

### Step 2

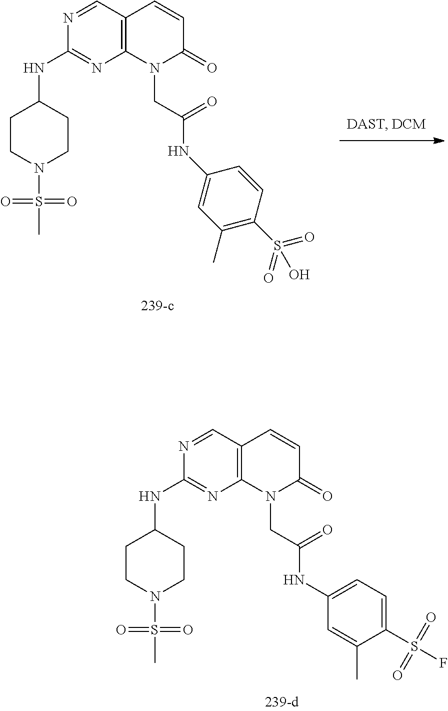 [SMILES: Cc1cc(NC(=O)Cn2c(=O)ccc3cnc(NC4CCN(S(C)(=O)=O)CC4)nc32)ccc1S(=O)(=O)O ; Cc1cc(NC(=O)Cn2c(=O)ccc3cnc(NC4CCN(S(C)(=O)=O)CC4)nc32)ccc1S(=O)(=O)F]

To a solution of 239-c (200 mg, 0.36 mmol) in dry DCM (30 mL) was added DAST (0.2 mL, 1.82 mmol) at 0° C. The reaction was stirred at room temperature for 2 h. Water was added thereto, the mixture was extracted with DCM (15 mL×2). The organic layer was extracted with THF (20 mL), and dried over anhydrous Na₂SO₄ filtered and concentrated in vacuo. The residue was purified by silica gel flash chromatography (gradient 0%-10% MeOH in DCM) to afford 239-d (80 mg, 40% yield) as a white solid. m/z: [M+H]⁺ Calcd for C₂₂H₂₆FN₆O₆S₂ 553.1; Found 553.0.

### Step 3

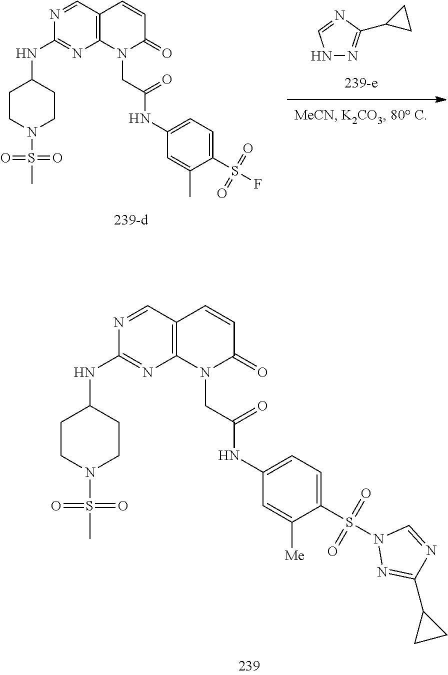 [SMILES: Cc1cc(NC(=O)Cn2c(=O)ccc3cnc(NC4CCN(S(C)(=O)=O)CC4)nc32)ccc1S(=O)(=O)F ; c1nc(C2CC2)n[nH]1 ; Cc1cc(NC(=O)Cn2c(=O)ccc3cnc(NC4CCN(S(C)(=O)=O)CC4)nc32)ccc1S(=O)(=O)n1cnc(C2CC2)n1]

To a solution of 239-d in MeCN (15 mL) was added 239-e and K2CO₃ (20 mg, 0.145 mmol). After 2 h at 80° C., the reaction was concentrated in vacuo. The residue was purified by reverse-phase chromatography (SilaSep™ C18 silica flash cartridge, 40%-60% MeCN in H₂O) to afford 239 (6.4 mg, 14% yield) as a white solid. m/z: [M+H]⁺ Calcd for C₂₇H₃₂N₉O₆S₂ 642.2; Found 642.2. ¹H NMR (400 MHz, DMSO) δ 10.97 (d, J=20.4 Hz, 1H), 9.23 (s, 1H), 8.66 (d, J=21.7 Hz, 1H), 8.09 (d, J=8.8 Hz, 1H), 7.98-7.66 (m, 4H), 6.33 (d, J=9.5 Hz, 1H), 5.05 (s, 2H), 3.85 (d, J=134.3 Hz, 1H), 3.52 (s, 2H), 2.79 (d, J=58.3 Hz, 3H), 2.45 (s, 5H), 2.01 (s, 1H), 1.81 (d, J=10.7 Hz, 2H), 1.48 (t, J=30.5 Hz, 2H), 0.95 (d, J=5.6 Hz, 2H), 0.82 (s, 2H).

### Synthesis of Compound 240

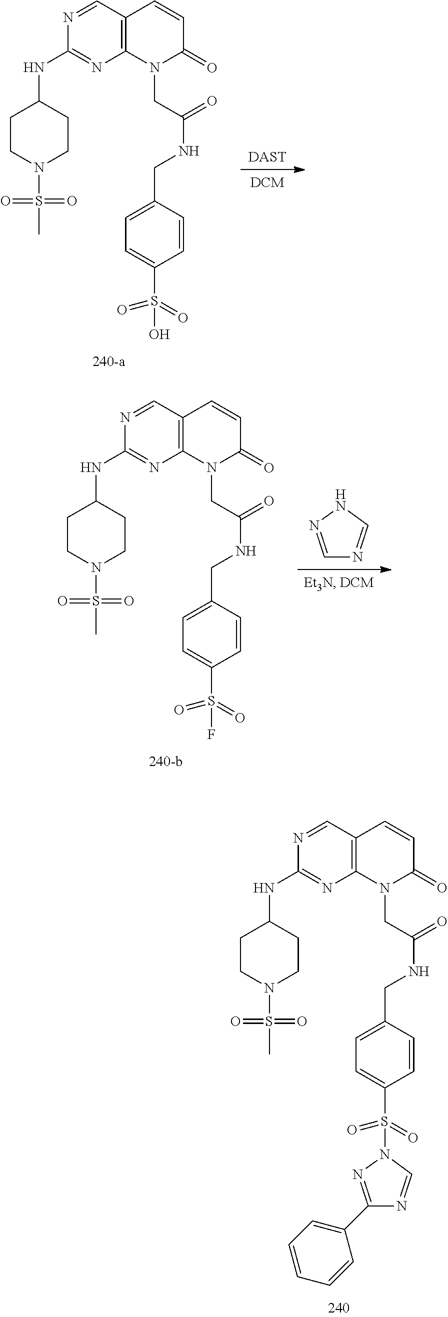

### Step 1

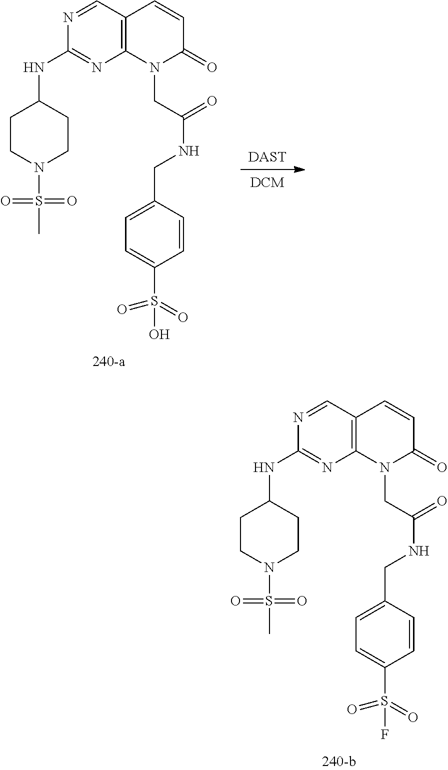

To a solution of 240-a (200 mg, 0.36 mmol) in DCM (50 mL) was added DAST (300 mg, 1.86 mmol) with stirring at 0° C. After stirred at rt for 16 h, the reaction mixture was concentrated and purified by silica gel flash chromatography (gradient 0%-10% MeOH in DCM) to afford 240-b (80 mg, 40% yield) as a yellow solid. m/z: [M+H]⁺ Calcd for C₂₂H₂₅FN₆O₆S₂ 552.1; Found 553.1.

### Step 2

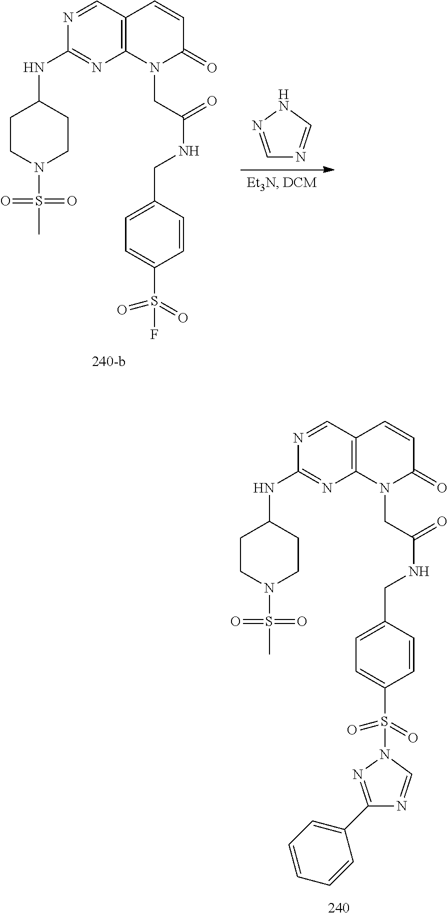

To a solution of 240-b (40 mg, 0.072 mmol) in THF (20 mL) and DMF (2 mL) was added K2CO₃ (30 mg, 0.22 mmol) and 3-phenyl-1H-1,2,4-triazole (20 mg, 0.14 mmol) with stirring at rt. After stirred at 60° C. for 5 h, the reaction mixture was concentrated and purified by reverse-phase chromatography (SilaSep™ C18 silica flash cartridge, 40%-60% MeCN in H₂O with 0.1% formic acid) to afford 240 (6.6 mg, 14% yield) as a white solid. m/z: [M+H]⁺ Calcd for C₃₀H₃₁N₉O₆S₂ 677.2; Found 678.5. ¹H NMR (400 MHz, DMSO) δ 9.45 (s, 1H), 8.81-8.58 (m, 2H), 8.09 (d, J=8.1 Hz, 2H), 7.99 (d, J=3.7 Hz, 2H), 7.84 (dd, J=64.7, 8.2 Hz, 2H), 7.60 (d, J=8.4 Hz, 2H), 7.51 (s, 3H), 6.29 (d, J=9.2 Hz, 1H), 4.88 (s, 2H), 4.38 (s, 2H), 3.85 (t, J=59.3 Hz, 1H), 3.54 (dd, J=35.9, 9.6 Hz, 2H), 2.83 (t, J=22.8 Hz, 5H), 1.91 (s, 2H), 1.56 (d, J=33.8 Hz, 2H).

### Synthesis of Compound 241

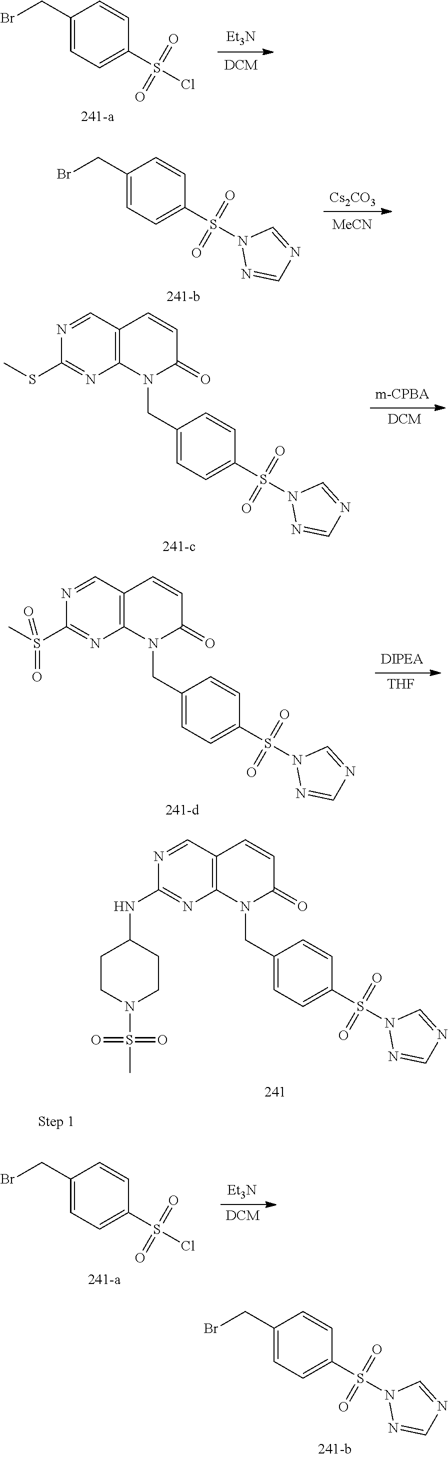

To a solution of 1H-1,2,4-triazole (0.06 mL, 1.00 mmol) and Et₃N (200 mg, 1.98 mmol) in DCM (10 mL) was added 241-a (300 mg, 1.11 mmol) with stirring at rt. After stirring at rt for 1 h, the reaction mixture was concentrated and purified by silica gel flash chromatography (gradient 0%-10% EtOAc in hexanes) to afford 241-b (260 mg, 86% yield) as a white solid. m/z: [M+H]⁺ Calcd for C₉H₈BrN₃O₂S 300.9; Found 302.0, 304.0.

### Step 2

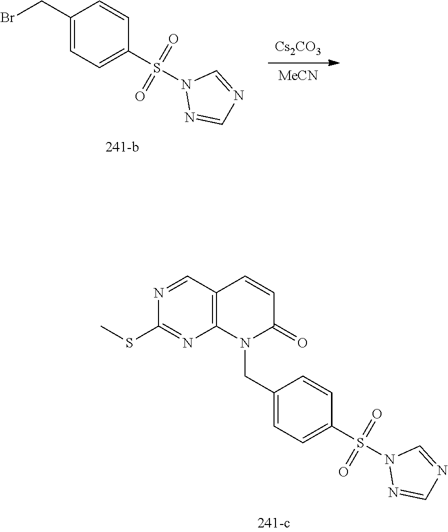

To a solution of 241-b (60 mg, 0.31 mmol) and K₂CO₃ (90 mg, 0.65 mmol) in MeCN (20 mL) was added 1-[4-(bromomethyl)benzenesulfonyl]-1H-1,2,4-triazole (100 mg, 0.33 mmol) with stirring at rt. After stirred at 80° C. for 1 h, the reaction mixture was concentrated and purified by silica gel flash chromatography (gradient 0%-50% EtOAc in hexanes) to afford 241-C (90 mg, 70% yield) as a white solid. m/z: [M+H]⁺ Calcd for C₁₇H₁₄N₆O₃S₂ 414.0; Found 415.1.

### Step 3

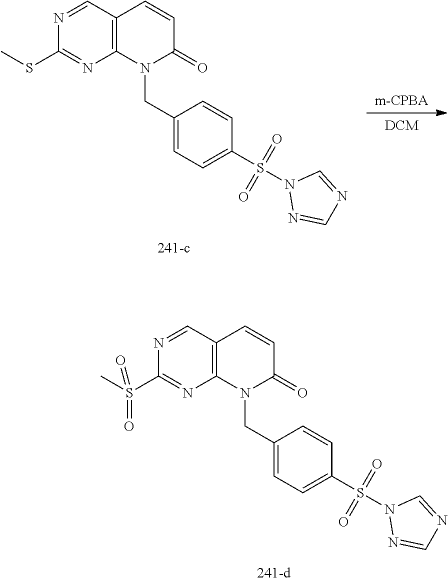

To a solution of 241-c (90 mg, 0.22 mmol) in DCM (20 mL) was added m-CPBA (100 mg, 0.58 mmol) with stirring at rt. After stirring 1 h, the reaction mixture was concentrated and purified by silica gel flash chromatography (gradient 0%-50% EtOAc in hexanes) to afford 241-d (85 mg, 88% yield) as a yellow solid. m/z: [M+H]⁺ Calcd for C₁₇H₁₄N₆O₅S₂ 446.0; Found 447.0.

### Step 4

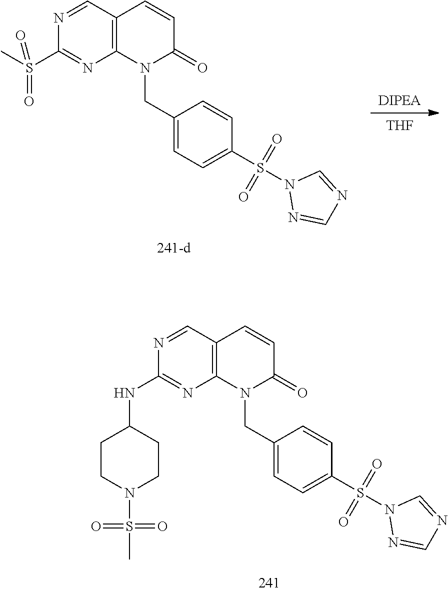 [SMILES: CS(=O)(=O)c1ncc2ccc(=O)n(Cc3ccc(S(=O)(=O)n4cncn4)cc3)c2n1 ; "DIPEA" ; CS(=O)(=O)N1CCC(Nc2ncc3ccc(=O)n(Cc4ccc(S(=O)(=O)n5cncn5)cc4)c3n2)CC1 ; "THF"]

To a solution of 241-d (85 mg, 0.19 mmol) and DIPEA (0.06 mL, 0.39 mmol) in THF (20 mL) was added 1-methanesulfonylpiperidin-4-amine (35 mg, 0.20 mmol) with stirring at rt. After stirred at 80° C. for 1 h, the reaction mixture was concentrated and purified by reverse-phase chromatography (SilaSep™ C18 silica flash cartridge, 40%-60% MeCN in H₂O with 0.1% formic acid) to afford 241 (10 mg, 10% yield) as a white solid. m/z: [M+H]⁺ Calcd for C₂₂H₂₄N₈O₅S₂ 544.1; Found 545.5. ¹H NMR (400 MHz, DMSO) δ 9.38 (s, 1H), 8.66 (d, J=30.3 Hz, 1H), 8.33 (d, J=8.2 Hz, 1H), 8.04 (d, J=8.4 Hz, 2H), 7.87 (dd, J=30.7, 8.5 Hz, 2H), 7.55 (t, J=10.1 Hz, 2H), 6.35 (dd, J=22.8, 9.2 Hz, 1H), 5.51 (d, J=10.3 Hz, 2H), 3.57 (s, 1H), 3.44 (s, 2H), 2.92-2.68 (m, 5H), 1.56 (m, 4H).

### Synthesis of Compound 242

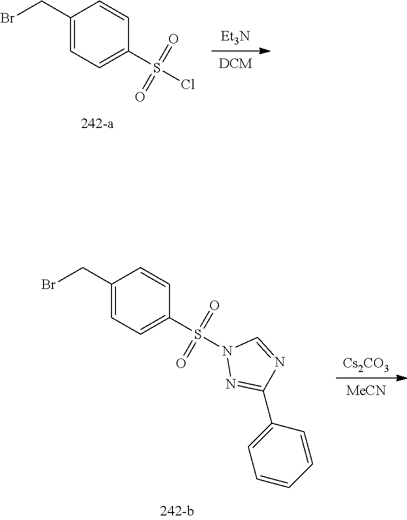

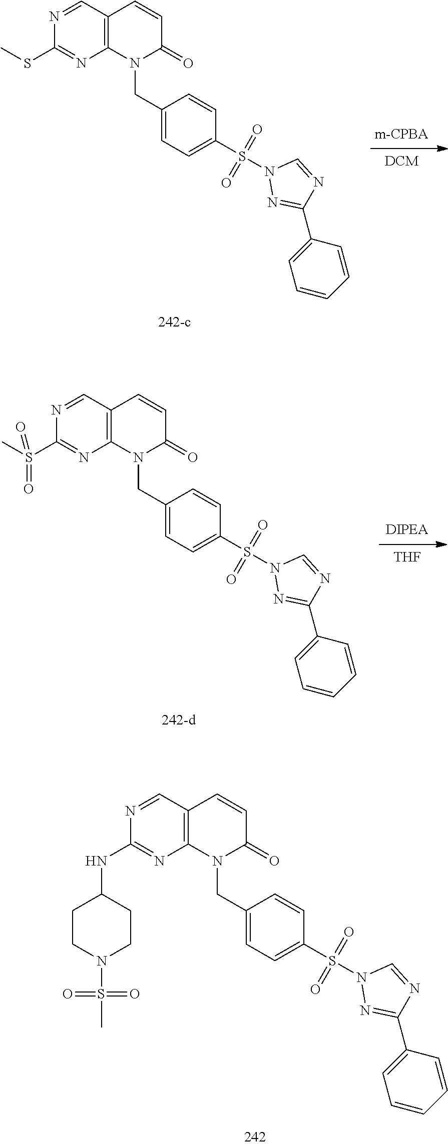

### Step 1

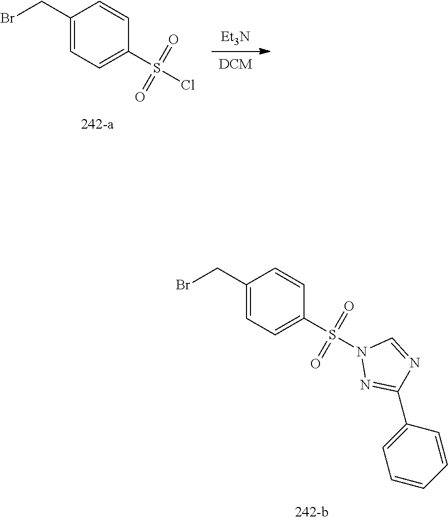

To a solution of 3-phenyl-1H-1,2,4-triazole (120 mg, 0.83 mmol) and Et₃N (200 mg, 1.98 mmol) in DCM (20 mL) was added 242-a (250 mg, 0.93 mmol) with stirring at rt. After stirring for 1 h, the reaction mixture was concentrated and purified by silica gel flash chromatography (gradient 0%-10% EtOAc in hexanes) to afford 242-b (280 mg, 90% yield) as a white solid. m/z: [M+H]⁺ Calcd for C₁₅H₁₂BrN₃O₂S 376.9; Found 378.0, 380.0.

### Step 2

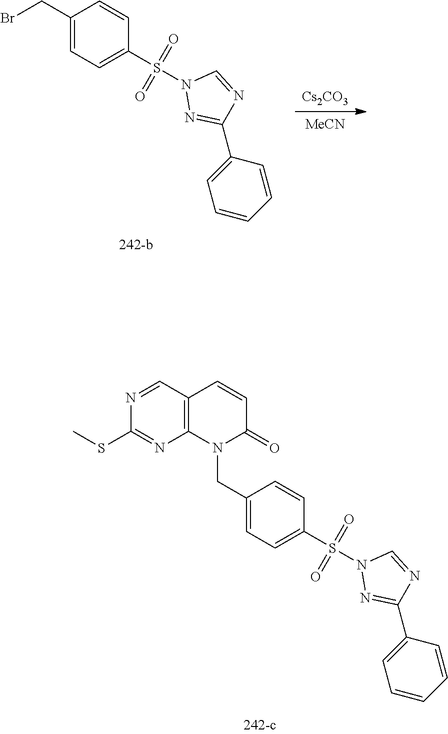

To a solution of 2-(methylsulfanyl)-7H,8H-pyrido[2,3-d]pyrimidin-7-one (140 mg, 0.73 mmol) and K2CO₃ (200 mg, 1.45 mmol) in MeCN (20 mL) was added 242-b (300 mg, 0.79 mmol) with stirring at rt. After stirred at 80° C. for 1 h, the reaction mixture was concentrated and purified by silica gel flash chromatography (gradient 0%-50% EtOAc in hexanes) to afford 242-c (260 mg, 73% yield) as a white solid. m/z: [M+H]⁺ Calcd for C₂₃H₁₈N₆O₃S₂490.1; Found 491.1.

### Step 3

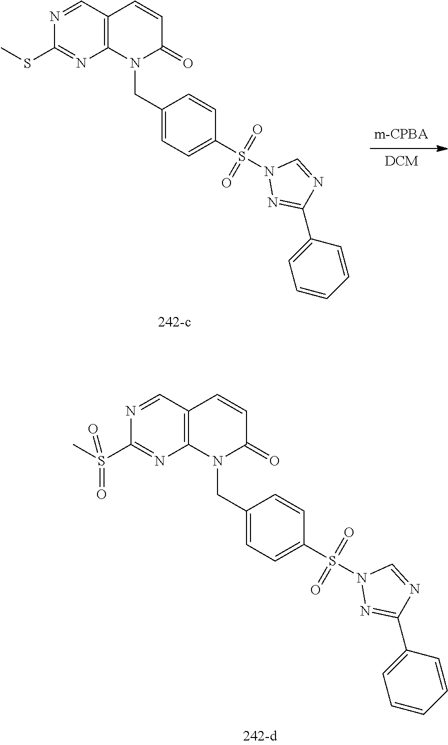

To a solution of 242-c (260 mg, 0.53 mmol) in DCM (20 mL) was added m-CPBA (300 mg, 1.74 mmol) with stirring at rt. After stirring 1 h, the reaction mixture was filtered to afford 242-d (230 mg, 83% yield) as a yellow solid. m/z: [M+H]⁺ Calcd for C₂₃H₂₀N₆O₅S₂ 524.0; Found 525.1.

### Step 4

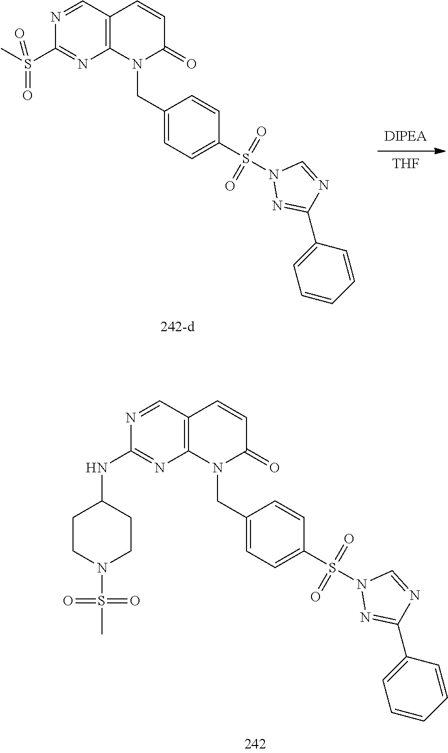 [SMILES: CS(=O)(=O)c1ncc2ccc(=O)n(Cc3ccc(S(=O)(=O)n4cnc(-c5ccccc5)n4)cc3)c2n1 ; "THF" ; CS(=O)(=O)N1CCC(Nc2ncc3ccc(=O)n(Cc4ccc(S(=O)(=O)n5cnc(-c6ccccc6)n5)cc4)c3n2)CC1]

To a solution of 242-d (230 mg, 0.44 mmol) and DIPEA (120 mg, 0.93 mmol) in THF (50 mL) was added 1-methanesulfonylpiperidin-4-amine (80 mg, 0.45 mmol) with stirring at rt. After stirred at 80° C. for 1 h, the reaction mixture was concentrated and purified by reverse-phase chromatography (SilaSep™ C18 silica flash cartridge, 40%-60% MeCN in H₂O with 0.1% formic acid) to afford 242 (110 mg, 40% yield) as a white solid. m/z: [M+H]⁺ Calcd for C₂₈H₂₈N₈O₅S₂ 620.1; Found 621.5. ¹H NMR (400 MHz, DMSO) δ 9.44 (s, 1H), 8.64 (d, J=37.2 Hz, 1H), 8.10 (t, J=8.1 Hz, 2H), 8.03-7.95 (m, 2H), 7.83 (dd, J=14.8, 8.5 Hz, 2H), 7.55 (d, J=8.4 Hz, 2H), 7.53-7.45 (m, 3H), 6.36 (d, J=9.3 Hz, 1H), 5.50 (d, J=11.0 Hz, 2H), 3.71 (t, J=77.5 Hz, 1H), 3.43 (dd, J=15.9, 7.4 Hz, 2H), 2.93-2.67 (m, 5H), 1.92-1.20 (m, 4H).

### Synthesis of Compound 243

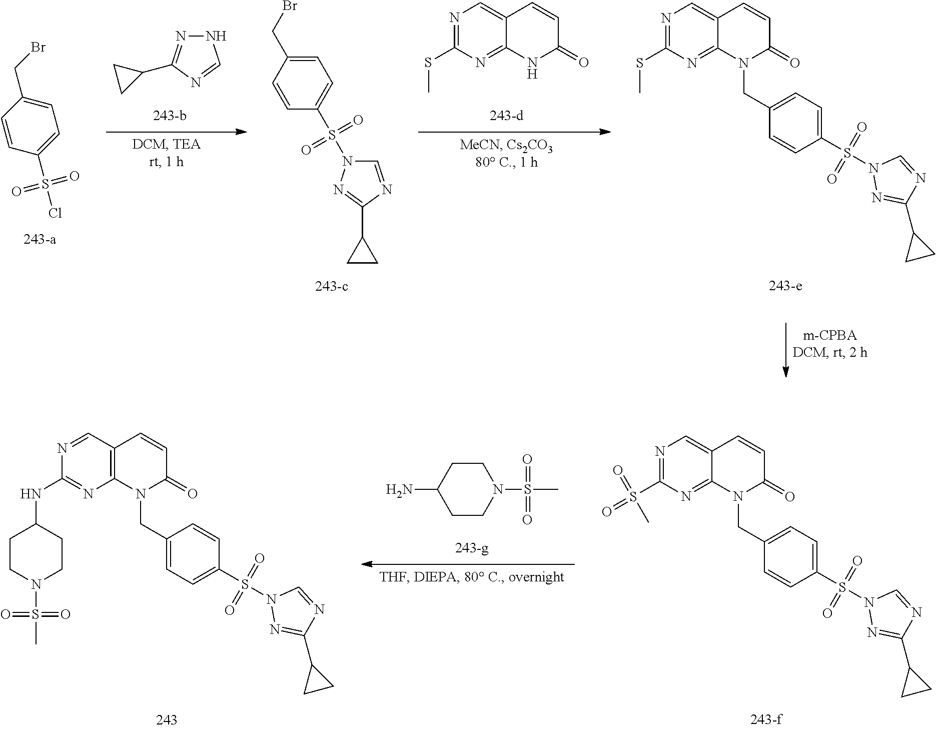

### Step 1

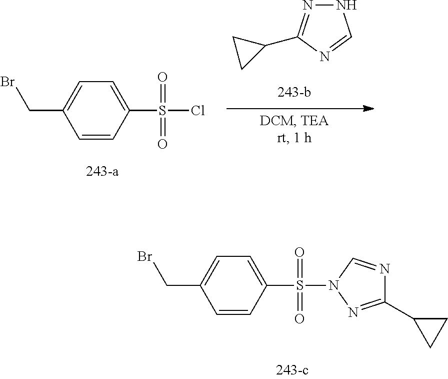 [SMILES: c1nc(C2CC2)n[nH]1 ; O=S(=O)(Cl)c1ccc(CBr)cc1 ; "DCM, TEArt, 1 h" ; O=S(=O)(c1ccc(CBr)cc1)n1cnc(C2CC2)n1]

To a solution of 243-a (110 mg, 1.008 mmol) in DCM (15 mL) was added 243-b (326.0 mg, 1.21 mmol) and TEA (0.28 mL, 2.02 mmol)). The reaction was stirred at rt for 1 h. And then the suspension was filtered and the filtrate is concentered in vacuo. The residue was purified by silica gel flash chromatography (gradient 0%-20% EtOAc in hexanes and was added 5% DCM) to afford 243-c (166 mg, 48.30%) as a white solid. m/z: [M+H]⁺ Calcd for C₁₇H₁₄N₆O₅S₂ 341.0; Found 341.9.

### Step 2

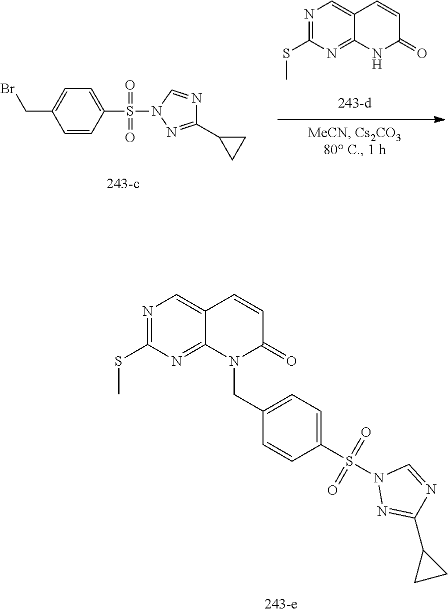 [SMILES: CSc1ncc2ccc(=O)[nH]c2n1 ; O=S(=O)(c1ccc(CBr)cc1)n1cnc(C2CC2)n1 ; "MeCN, Cs2CO380° C., 1 h" ; CSc1ncc2ccc(=O)n(Cc3ccc(S(=O)(=O)n4cnc(C5CC5)n4)cc3)c2n1]

To a solution of 243-c (166 mg, 0.49 mmol) in MeCN (15 mL) was added 243-d (93.7 mg, 0.485 mmol) and Cs₂CO₃ (284.5 mg, 0.87 mmol)). The reaction was stirred at 80° C. for 1 h. And then the suspension is filtered and the filtra is concentered in vacuo. The residue was purified by silica gel flash chromatography (gradient 0%-20% EtOAc in DCM) to afford 243-e (200 mg, 90.71%) as a white solid. m/z: [M+H]⁺ Calcd for C₂₀H₁₈N₆O₃S₂ 454.1; Found 455.0.

### Step 3

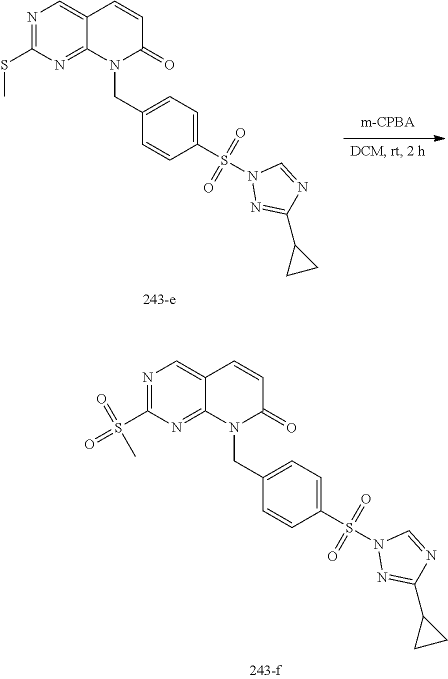 [SMILES: CSc1ncc2ccc(=O)n(Cc3ccc(S(=O)(=O)n4cnc(C5CC5)n4)cc3)c2n1 ; "DCM, rt, 2 h" ; CS(=O)(=O)c1ncc2ccc(=O)n(Cc3ccc(S(=O)(=O)n4cnc(C5CC5)n4)cc3)c2n1]

To a solution of 243-e (243 mg, 0.54 mmol) was added m-CPBA (0.41 g, 1.34 mmol) at 0° C. The reaction was stirred at rt for 2 h. And then the suspension is filtered and the filtrate is concentered in vacuo. The residue was purified by silica gel flash chromatography (gradient 0%-35% EtOAc in DCM) to afford 243-f (169 mg, 64.97%) as a yellow oil. m/z: [M+H]+ Calcd for C₂₀H₁₈N₆O₅S₂ 486.1; Found 487.1.

### Step 4

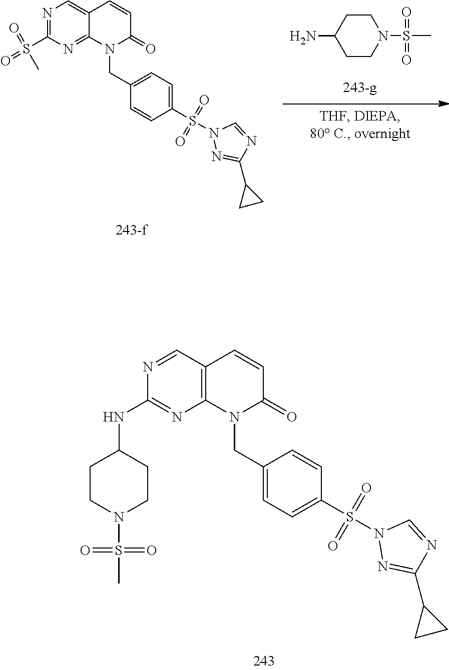 [SMILES: CS(=O)(=O)c1ncc2ccc(=O)n(Cc3ccc(S(=O)(=O)n4cnc(C5CC5)n4)cc3)c2n1 ; CS(=O)(=O)N1CCC(N)CC1 ; "THF, DIEPA,80° C., overnight" ; CS(=O)(=O)N1CCC(Nc2ncc3ccc(=O)n(Cc4ccc(S(=O)(=O)n5cnc(C6CC6)n5)cc4)c3n2)CC1]

To a solution of 243-f (169 mg, 0.35 mmol) in THF (10 mL) was added DIPEA (89.6 mg, 0.69 mmol) and 243-g (74.3 mg, 0.41 mmol). The reaction was stirred at 80° C. overnight. And then the suspension is filtered and the filtrate was concentered in vacuo.

The residue was purified by reverse phase chromatography (SilaSep™ C18 silica flash cartridge, 40%-60% MeCN in H₂O) to afford 243 (95.7 mg, 47.12%) as a white solid. m/z: [M+H]⁺ Calcd for C₂₅H₂₈N₈O₅S₂ 584.2; Found 585.5. 1H NMR (400 MHz, DMSO) δ 9.16 (d, J=7.8 Hz, 1H), 8.62 (s, 1H), 8.01 (d, J=8.3 Hz, 2H), 7.86 (dd, J=29.2, 8.5 Hz, 2H), 7.53 (d, J=8.4 Hz, 2H), 6.38 (d, J=9.3 Hz, 1H), 5.52 (s, 2H), 3.72 (t, J=76.9 Hz, 1H), 3.46 (d, J=12.7 Hz, 2H), 2.89 (d, J=19.3 Hz, 3H), 2.81-2.66 (m, 2H), 2.03-1.96 (m, 1H), 1.48 (dd, J=75.1, 9.8 Hz, 4H), 0.94 (dd, J=8.1, 2.6 Hz, 2H), 0.85-0.64 (m, 2H).

### Synthesis of Compound 244

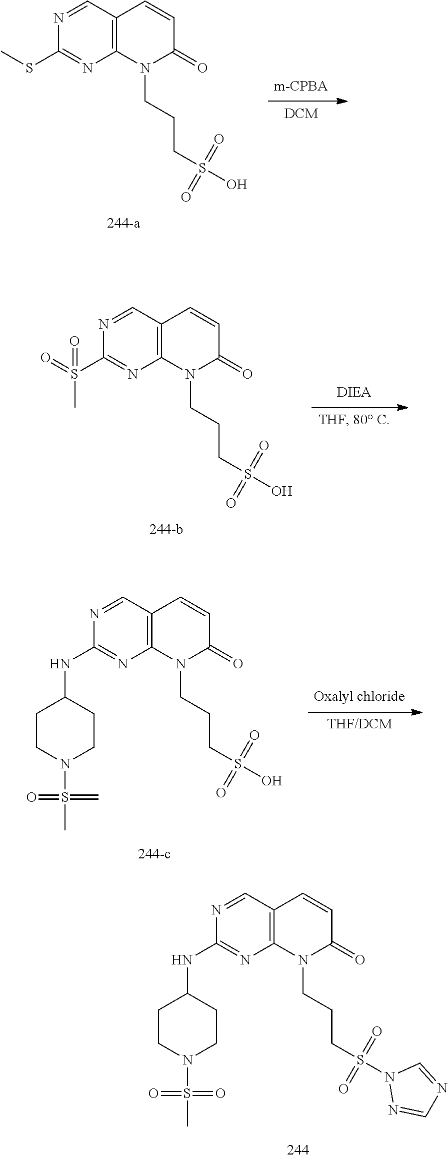

Step 1

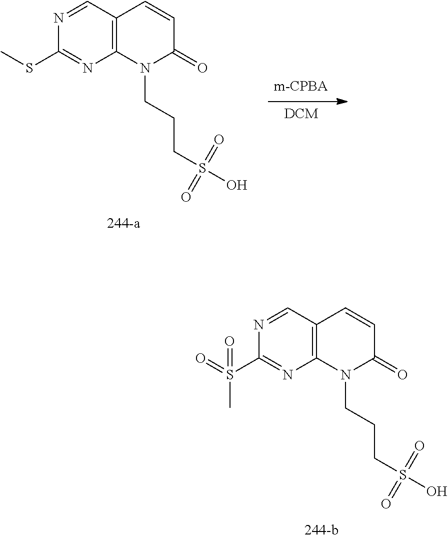

To a solution of 244-a (600 mg, 1.90 mmol) in DCM (30 mL) was added m-CPBA (980 mg, 5.68 mmol) with stirring at rt. After stirring for 16 h, the reaction mixture was filtered and dried to afford 244-b (500 mg, 75.66%) as a white solid. m/z: [M+H]⁺ Calcd for C₁₁H₁₃N₃O₆S₂ 347.0; Found 348.1.

### Step 2

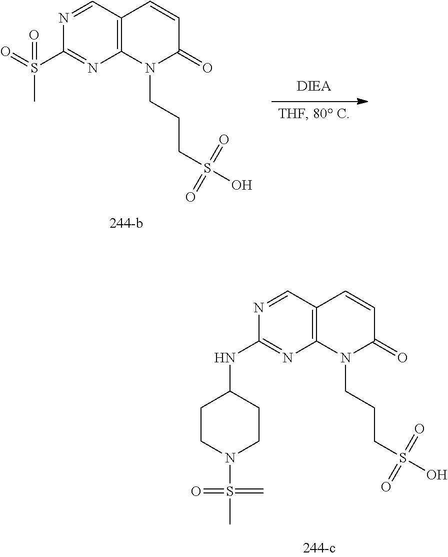 [SMILES: CS(=O)(=O)c1ncc2ccc(=O)n(CCCS(=O)(=O)O)c2n1 ; "THF, 80° C." ; C=S(C)(=O)N1CCC(Nc2ncc3ccc(=O)n(CCCS(=O)(=O)O)c3n2)CC1]

To a solution of 244-b (500 mg, 1.44 mmol) and 1-methanesulfonylpiperidin-4-amine (256.5 mg, 1.44 mmol) in THF (20 mL) was added DIEA (400 mg, 3.09 mmol) with stirring at rt. After stirred at 80° C. for 16 h, the reaction mixture was concentrated and purified by reverse-phase chromatography (SilaSep™ C18 silica flash cartridge, 40%-60% MeOH in H₂O with 0.1% formic acid) to afford 244-c (180 mg, 28.07%) as a yellow solid. m/z: [M+H]⁺ Calcd for C₁₆H₂₃N₅O₆S₂ 445.1; Found 446.1.

### Step 3

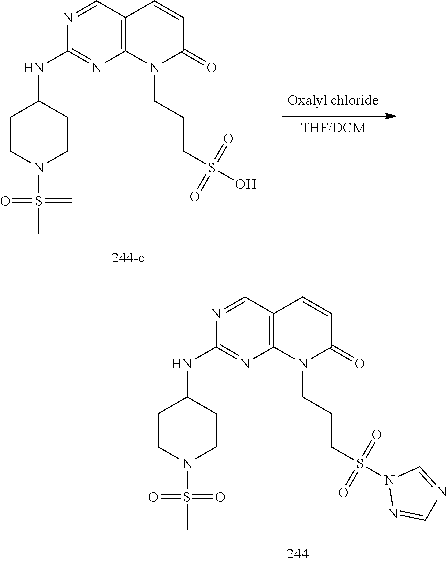 [SMILES: C=S(C)(=O)N1CCC(Nc2ncc3ccc(=O)n(CCCS(=O)(=O)O)c3n2)CC1 ; "THF/DCM" ; CS(=O)(=O)N1CCC(Nc2ncc3ccc(=O)n(CCCS(=O)(=O)n4cncn4)c3n2)CC1]

To a solution of 244-c (90 mg, 0.20 mmol) in THF (5 mL) and DCM (5 mL) was added oxalyl chloride (55 mg, 0.43 mmol) with stirring at rt. After 1 h, the reaction mixture was concentrated. The residue was added to the solution of 1H-1,2,4-triazole (30 mg, 0.43 mmol) and Et₃N (40 mg, 0.4 mmol) in DCM (5 mL). After 1 h, the reaction mixture was concentrated and purified by reverse-phase chromatography (SilaSep™ C18 silica flash cartridge, 40%-60% MeOH in H₂O with 0.1% formic acid) to afford 244 (15.1 mg, 0.030 mmol, 15.05%) as a white solid. m/z: [M+H]⁺ Calcd for C₁₈H₂₄N₈O₅S₂ 496.1; Found 497.1. ¹H NMR (400 MHz, DMSO) δ 9.19 (s, 1H), 8.60 (s, 1H), 8.39 (s, 1H), 8.04-7.62 (m, 2H), 6.26 (t, J=9.3 Hz, 1H), 4.28 (m, 2H), 4.11-3.85 (m, 3H), 3.57 (d, J=12.2 Hz, 2H), 2.94-2.76 (m, 5H), 1.98 (s, 4H), 1.66-1.53 (m, 2H).

### Synthesis of Compound 246

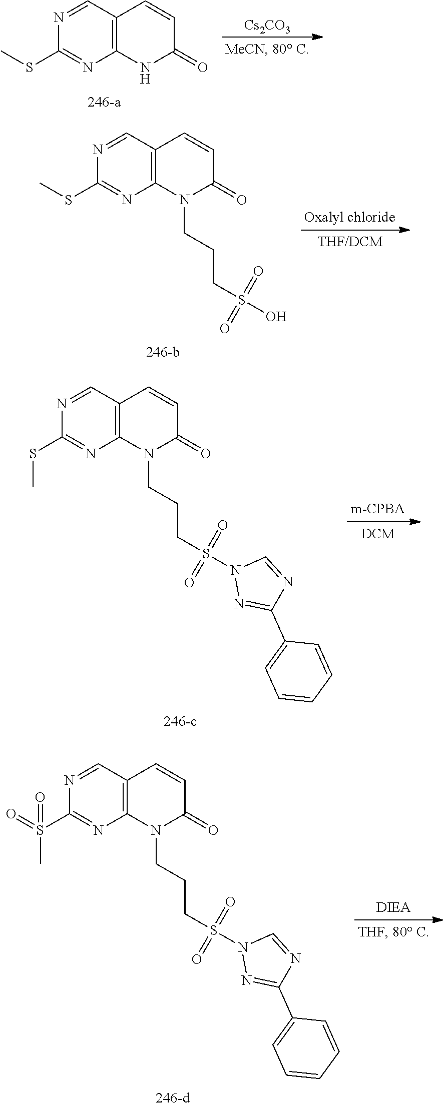

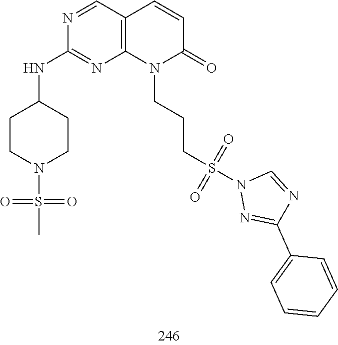 [SMILES: CS(=O)(=O)N1CCC(Nc2ncc3ccc(=O)n(CCCS(=O)(=O)n4cnc(-c5ccccc5)n4)c3n2)CC1]

### Step 1

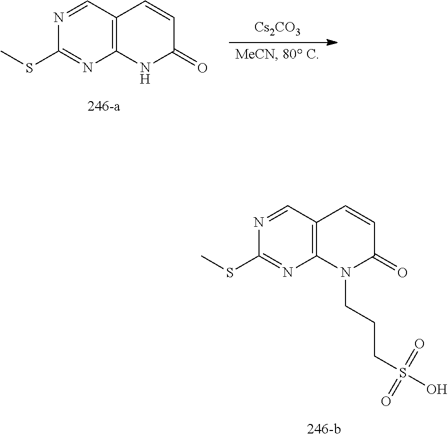

To a solution of 246-a (600 mg, 3.11 mmol) and sodium 3-bromopropane-1-sulfonate (750 mg, 3.69 mmol) in MeCN (20 mL) was added Cs₂CO₃ (1.5 g, 4.60 mmol) with stirring at rt. After stirred at 80° C. for 2 h, the reaction mixture was concentrated and purified by reverse-phase chromatography (SilaSep™ C18 silica flash cartridge, 40%-60% MeOH in H₂O with 0.1% formic acid) to afford 246-b (880 mg, 89.86%) as a yellow solid. m/z: [M+H]⁺ Calcd for C₁₁H₁₃N₃O₄S₂ 315.0; Found 316.1.

### Step 2

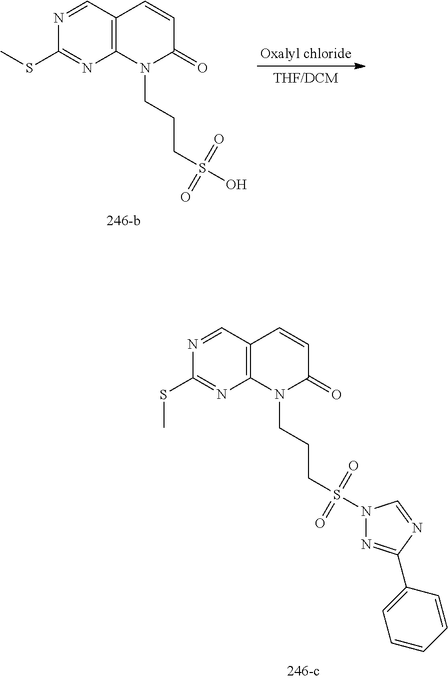 [SMILES: CSc1ncc2ccc(=O)n(CCCS(=O)(=O)O)c2n1 ; CSc1ncc2ccc(=O)n(CCCS(=O)(=O)n3cnc(-c4ccccc4)n3)c2n1]

To a solution of 246-b (200 mg, 0.63 mmol) and DMF (46 mg, 0.63 mmol) in DCM (10 mL) and THF (10 mL) was added oxalyl chloride (160 mg, 1.26 mmol) with stirring at rt. After 1 h, the reaction mixture was concentrated and purified by silica gel flash chromatography (gradient 0%-60% EtOAc in hexanes) to afford 246-c (150 mg, 53.45%) as a light yellow solid. [M+Na]⁺ m/z: Calcd for C₁₉H₁₈N₆O₃S₂ 442.1; Found 464.7.

### Step 3

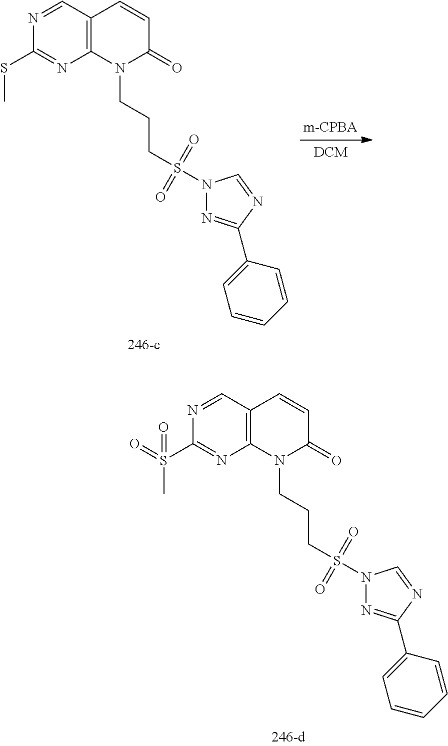 [SMILES: CSc1ncc2ccc(=O)n(CCCS(=O)(=O)n3cnc(-c4ccccc4)n3)c2n1 ; CS(=O)(=O)c1ncc2ccc(=O)n(CCCS(=O)(=O)n3cnc(-c4ccccc4)n3)c2n1]

To a solution of 246-c (150 mg, 0.34 mmol) in DCM (10 mL) was added m-CPBA (180 mg, 1.04 mmol) with stirring at rt. After stirring for 1 h, the reaction mixture was concentrated and purified by silica gel flash chromatography (gradient 0%-50% EtOAc in hexanes) to afford 246-d (90 mg, 55.96%) as a yellow solid. m/z: [M+H]⁺ Calcd for C₁₉H₁₈N₆O₅S₂ 474.1; Found 475.0.

### Step 4

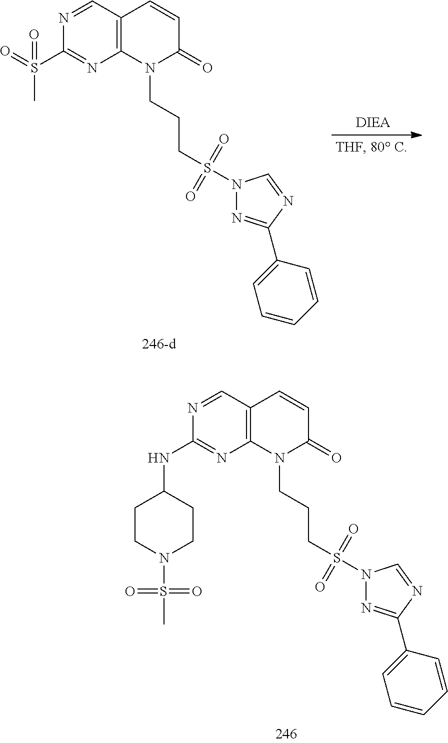 [SMILES: CS(=O)(=O)c1ncc2ccc(=O)n(CCCS(=O)(=O)n3cnc(-c4ccccc4)n3)c2n1 ; CS(=O)(=O)N1CCC(Nc2ncc3ccc(=O)n(CCCS(=O)(=O)n4cnc(-c5ccccc5)n4)c3n2)CC1]

To a solution of 246-d (90 mg, 0.19 mmol) and DIEA (60 mg, 0.46 mmol) in THF (10 mL) was added 1-methanesulfonylpiperidin-4-amine (50 mg, 0.28 mmol) with stirring at rt. After stirred at 80° C. for 1 h, the reaction mixture was concentrated and purified by reverse-phase chromatography (SilaSep™ C18 silica flash cartridge, 40%-60% MeCN in H₂O with 0.1% formic acid) to afford 246 (19.6 mg, 18.05%) as a white solid. m/z: [M+H]⁺ Calcd for C₂₄H₂₈N₈O₅S₂ 572.2; Found 573.5. ¹H NMR (400 MHz, DMSO) δ 9.24 (s, 1H), 8.51 (s, 1H), 8.00 (dd, J=6.6, 3.1 Hz, 2H), 7.79 (dd, J=99.1, 8.5 Hz, 2H), 7.59-7.48 (m, 3H), 6.22 (d, J=9.3 Hz, 1H), 4.31 (dd, J=21.8, 15.5 Hz, 2H), 4.01 (dd, J=24.5, 17.0 Hz, 3H), 3.55 (d, J=12.1 Hz, 2H), 2.98-2.85 (m, 5H), 2.08 (dd, J=9.0, 5.7 Hz, 2H), 1.92 (d, J=10.1 Hz, 2H), 1.54 (d, J=10.6 Hz, 2H)

### Synthesis of Compound 249

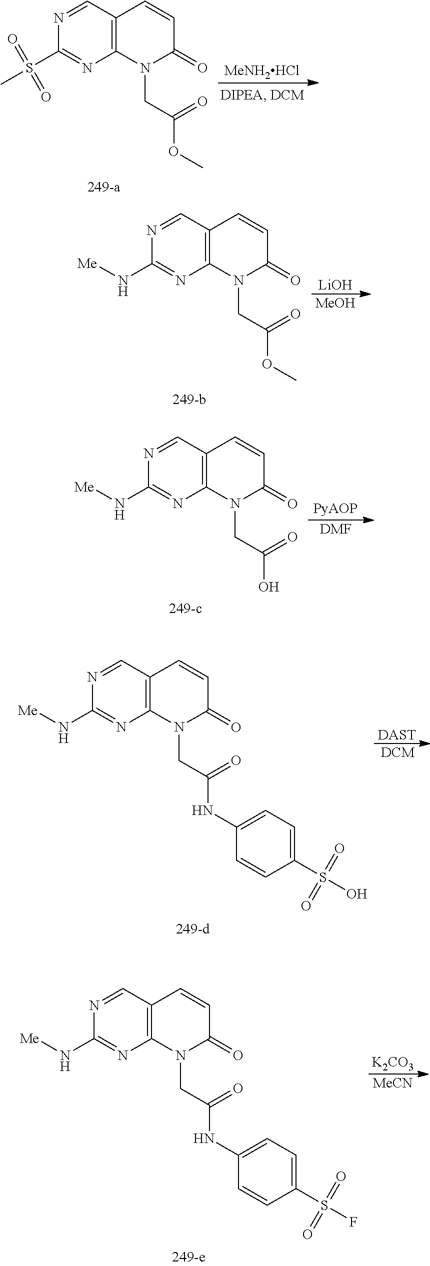

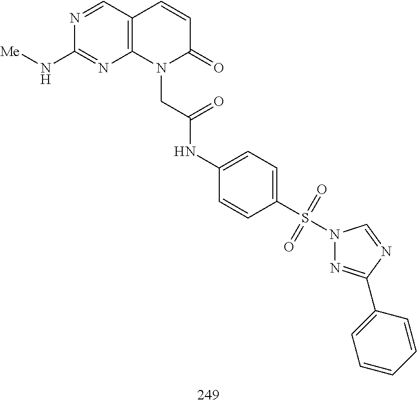 [SMILES: CNc1ncc2ccc(=O)n(CC(=O)Nc3ccc(S(=O)(=O)n4cnc(-c5ccccc5)n4)cc3)c2n1]

### Step 1

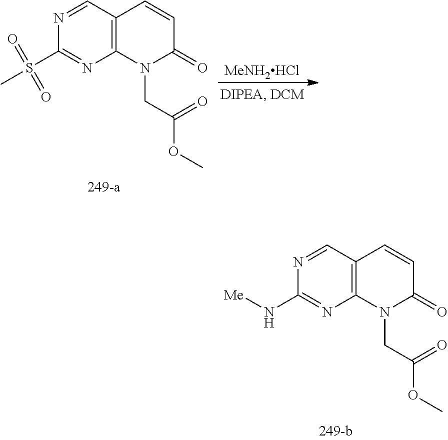

To a solution of 249-a (480 mg, 1.62 mmol) and DIEA (600 mg, 4.64 mmol) in DCM (20 mL) was added methylamine hydrochloride (150 mg, 2.22 mmol) with stirring at rt. After stirring for 16 h, the reaction mixture was concentrated and purified by silica gel flash chromatography (gradient 0%-80% EtOAc in hexanes) to afford 249-b (350 mg, 87.32%) as a yellow solid. m/z: [M+H]⁺ Calcd for C₁₁H₁₂N₄O₃ 248.1; Found 249.2.

### Step 2

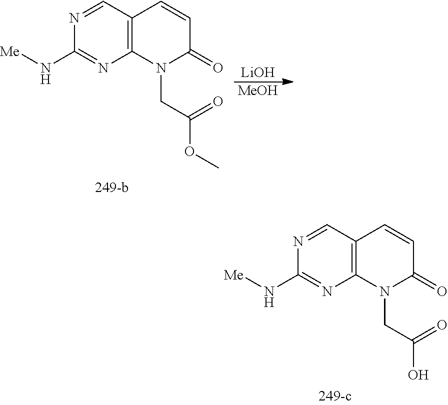

To a solution of 249-b (350 mg, 1.41 mmol) in MeOH (20 mL) was added aq. NaOH (200 mg, 5.00 mmol) with stirring at rt. After stirring for 2 h, the reaction mixture was concentrated and adjusted pH to 6. The mixture was purified by reverse-phase chromatography (SilaSep™ C18 silica flash cartridge, 40%-60% MeOH in H₂O with 0.1% formic acid) to afford 249-c (300 mg, 90.84%) as a white solid. m/z: [M+H]⁺ Calcd for C₁₀H₁₀N₄O₃ 234.1; Found 235.2.

### Step 3

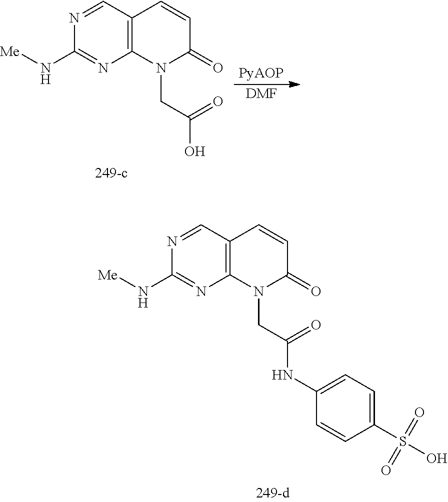

To a solution of 249-c (300 mg, 1.28 mmol), Et₃N (260 mg, 2.57 mmol) and 4-aminobenzene-1-sulfonic acid (250 mg, 1.44 mmol) in DMF (8 mL) was added PyAOP (820 mg, 1.57 mmol) with stirring at rt. After stirred at rt for 1 h, the reaction mixture was concentrated and purified by reverse-phase chromatography (SilaSep™ C18 silica flash cartridge, 40%-60% MeCN in H₂O with 0.1% formic acid) to afford 249-d (230 mg, 46.12%) as a yellow solid. m/z: [M+H]⁺ Calcd for C₁₆H₁₅N₅O₅S 389.1; Found 390.4.

### Step 4

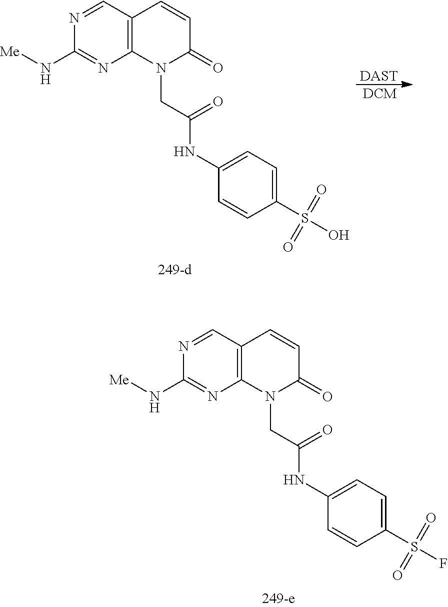

To a solution of 249-d (230 mg, 0.59 mmol) in DCM (50 mL) was added DAST (0.5 mL, 3.78 mmol) with stirring at 0° C. After stirred at rt for 2 h, the reaction mixture was poured into water and extracted with DCM (30 mL×2). The organic layer was washed with brine (30 mL), dried over anhydrous Na₂SO₄, filtered and concentrated in vacuo. The residue was purified by silicagel flash chromatography (gradient 0%-10% EtOAc in hexanes) to afford 249-e (150 mg, 64.89%) as a yellow solid. m/z: [M+H]⁺ Calcd for C₁₆H₁₄FN₅O₄S 391.1; Found 392.4.

### Step 5

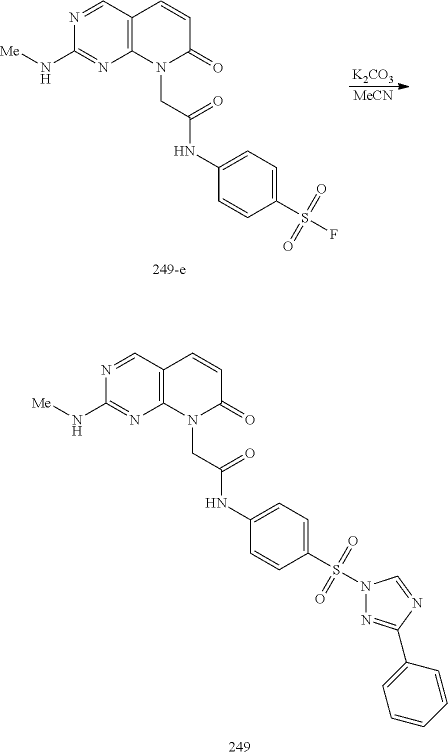 [SMILES: "MeCN" ; CNc1ncc2ccc(=O)n(CC(=O)Nc3ccc(S(=O)(=O)F)cc3)c2n1 ; "K2CO3" ; CNc1ncc2ccc(=O)n(CC(=O)Nc3ccc(S(=O)(=O)n4cnc(-c5ccccc5)n4)cc3)c2n1]

To a solution of 249-e (40 mg, 0.10 mmol) and 3-phenyl-1H-1,2,4-triazole (20 mg, 0.14 mmol) in MeCN (10 mL) was added K₂CO₃ (30 mg, 0.22 mmol) with stirring at rt. After stirred at 80° C. for 24 h, the reaction mixture was concentrated and purified by reverse-phase chromatography (SilaSep™ C18 silica flash cartridge, 40%-60% MeCN in H₂O with 0.1% formic acid) to afford 249 (5.5 mg, 10.42%) as a white solid. m/z: [M+H]⁺ Calcd for C₂₄H₂₀N₈O₄S 516.1; Found 517.0. ¹H NMR (400 MHz, DMSO) δ 11.05 (s, 1H), 9.42 (s, 1H), 8.59 (s, 1H), 8.11 (d, J=9.0 Hz, 2H), 7.99 (dd, J=6.7, 3.0 Hz, 2H), 7.87 (d, J=9.0 Hz, 2H), 7.79 (t, J=8.1 Hz, 2H), 7.52-7.49 (m, 3H), 6.29 (d, J=9.3 Hz, 1H), 5.05 (m, 2H), 2.74 (in, 3H).

### Synthesis of Compound 252

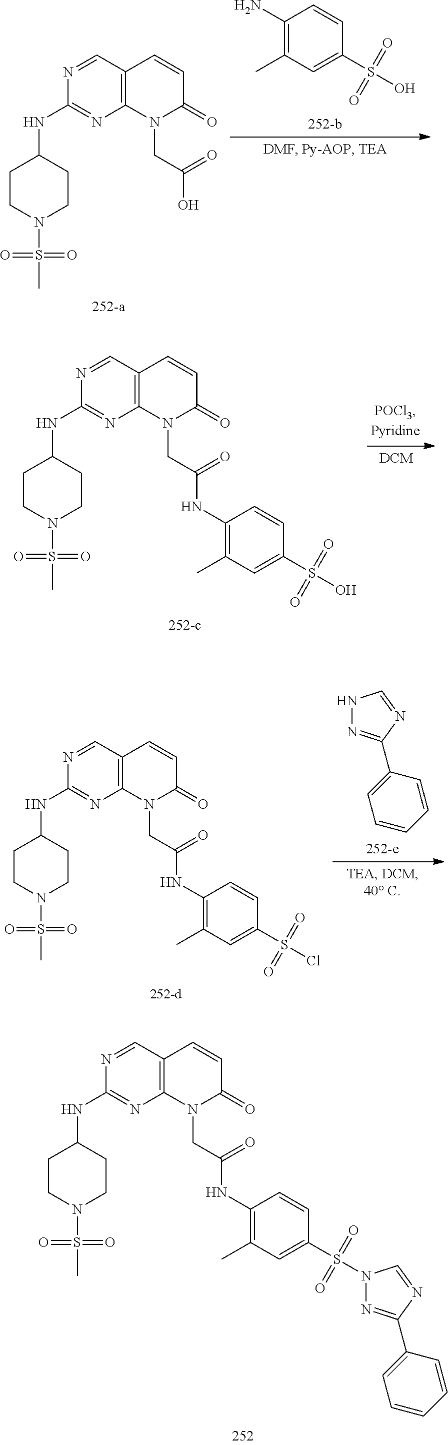 [SMILES: CS(=O)(=O)N1CCC(Nc2ncc3ccc(=O)n(CC(=O)O)c3n2)CC1 ; Cc1cc(S(=O)(=O)O)ccc1N ; Cc1cc(S(=O)(=O)O)ccc1NC(=O)Cn1c(=O)ccc2cnc(NC3CCN(S(C)(=O)=O)CC3)nc21 ; Cc1cc(S(=O)(=O)Cl)ccc1NC(=O)Cn1c(=O)ccc2cnc(NC3CCN(S(C)(=O)=O)CC3)nc21 ; c1ccc(-c2nc[nH]n2)cc1 ; Cc1cc(S(=O)(=O)n2cnc(-c3ccccc3)n2)ccc1NC(=O)Cn1c(=O)ccc2cnc(NC3CCN(S(C)(=O)=O)CC3)nc21]

### Step 1

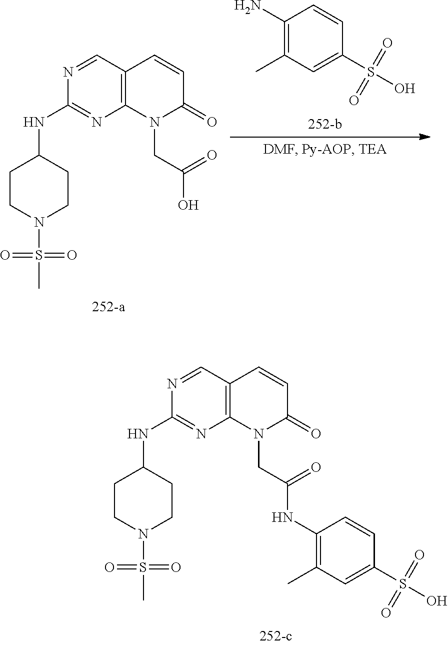 [SMILES: CS(=O)(=O)N1CCC(Nc2ncc3ccc(=O)n(CC(=O)O)c3n2)CC1 ; Cc1cc(S(=O)(=O)O)ccc1N ; Cc1cc(S(=O)(=O)O)ccc1NC(=O)Cn1c(=O)ccc2cnc(NC3CCN(S(C)(=O)=O)CC3)nc21]

To a solution of 252-a (300.0 mg, 0.78 mmol) in DMF (10.0 mL), 252-b (176.0 mg, 0.94 mmol), Py-AOP (614.0 mg, 1.18 mmol), TEA (158.0 mg, 1.57 mmol) was added. The resulting mixture stirred at rt for 3 h. extracted with DCM (2×50 mL), the organic layer was concentrated under reduced pressure to afford the crude product, which was purified by flash chromatography on silica gel (DCM/MeOH=1/0 to 4/1) to give 252-c (400.0 mg, 92.4%) as a white solid. m/z: [M+H]⁺ Calcd for C₂₂H₂₆N₆O₇S 2549.1; Found 549.1.

### Step 2

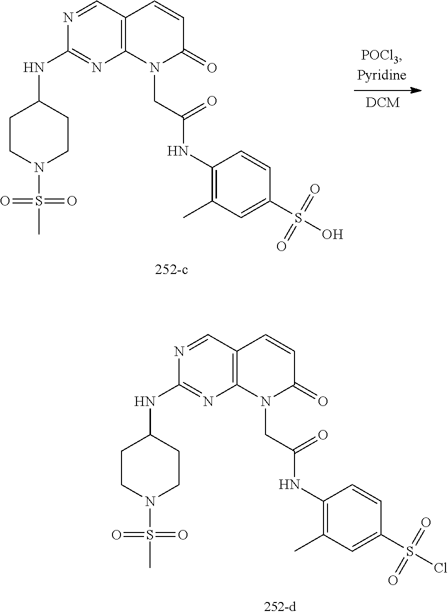 [SMILES: Cc1cc(S(=O)(=O)O)ccc1NC(=O)Cn1c(=O)ccc2cnc(NC3CCN(S(C)(=O)=O)CC3)nc21 ; Cc1cc(S(=O)(=O)Cl)ccc1NC(=O)Cn1c(=O)ccc2cnc(NC3CCN(S(C)(=O)=O)CC3)nc21]

To a solution of 252-c (300.0 mg, 0.54 mmol) in DCM (10.0 mL), POCl₃ (1.0 ml), pyridine (0.5 ml) was added. The resulting mixture stirred at rt for 1 h. The mixture was concentrated under reduced pressure, then extracted with DCM (2×50 mL). The organic layer was concentrated under reduced pressure to afford the crude product, which was purified by flash chromatography on silica gel (DCM/MeOH=1/1 to 10/1) to give 252-d (300.0 mg, 97.1%) as an oil. m/z: [M+H]⁺ Calcd for C₂₂H₂₅ClN₆O₆S₂ 568.1; Found 569.1.

### Step 3

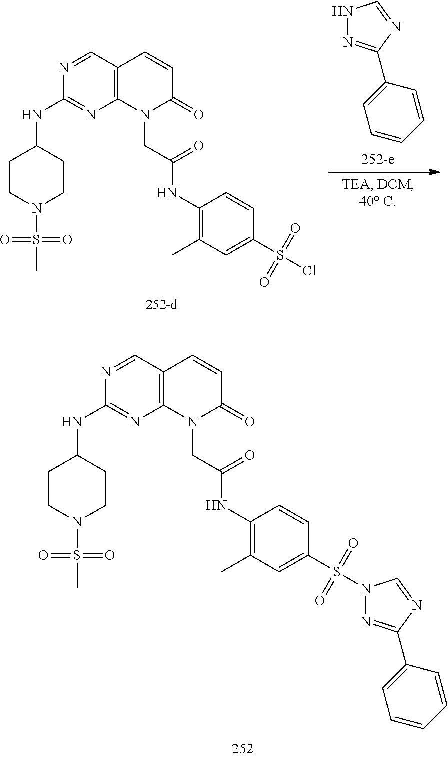 [SMILES: Cc1cc(S(=O)(=O)Cl)ccc1NC(=O)Cn1c(=O)ccc2cnc(NC3CCN(S(C)(=O)=O)CC3)nc21 ; c1ccc(-c2nc[nH]n2)cc1 ; Cc1cc(S(=O)(=O)n2cnc(-c3ccccc3)n2)ccc1NC(=O)Cn1c(=O)ccc2cnc(NC3CCN(S(C)(=O)=O)CC3)nc21]

To a solution of 252-d (100.0 mg, 0.17 mmol) in DCM (3.0 mL), 252-e (26.0 mg, 0.17 mol), TEA (36.0 mg, 0.35 mmol) was added. The resulting mixture stirred at rt for 1 h. The mixture was extracted with DCM (2×50 mL) and the organic layer was concentrated under reduced pressure to afford the crude product, which was purified TLC (DCM/MeOH=10/1) to give 252(5.0 mg, 4.2%) as white solid. m/z: [M+H]⁺ Calcd for C₃₀H₃₁N₉O₆S₂ 677.1; Found 678.1. ¹H NMR (400 MHz, DMSO) δ 9.95 (s, 1H), 9.41 (s, 1H), 8.63 (s, 1H), 8.10-8.02 (m, 2H), 8.01-7.92 (m, 4H), 7.80 (d, J=9.4 Hz, 1H), 7.53-7.47 (m, 3H), 6.32 (d, J=9.3 Hz, 1H), 5.15 (d, J=18.8 Hz, 2H), 2.86 (s, 1H), 2.65 (d, J=28.1 Hz, 3H), 2.43 (s, 3H), 2.40 (s, 2H), 2.00-1.76 (m, 3H), 1.44 (d, J=10.0 Hz, 3H).

### Synthesis of Compound 254

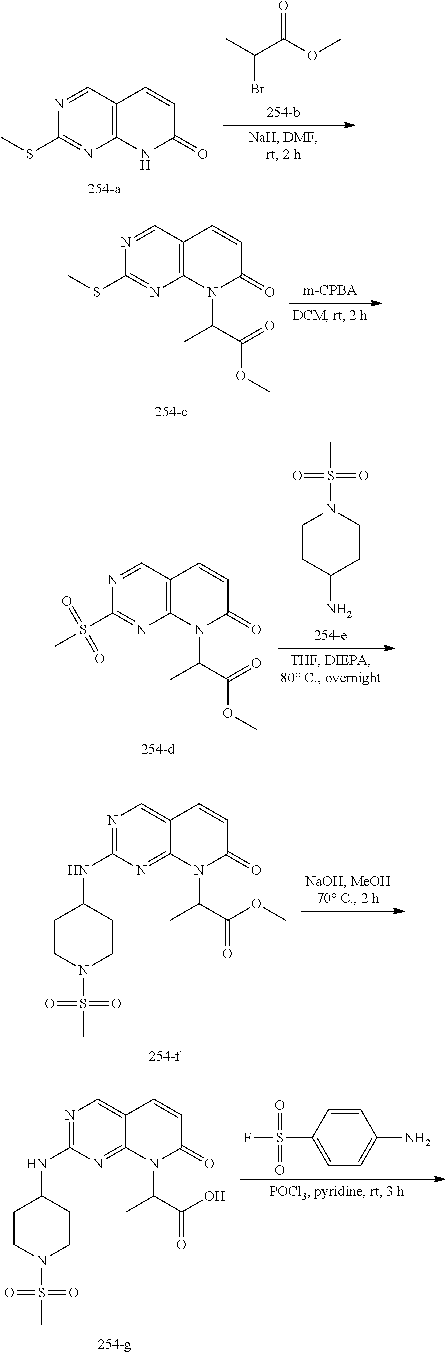

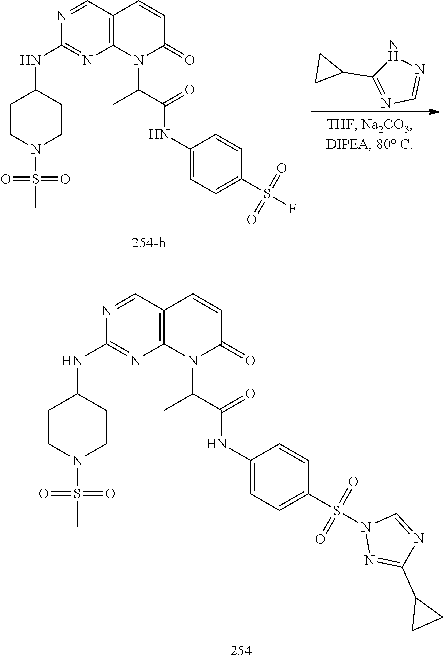 [SMILES: CC(C(=O)Nc1ccc(S(=O)(=O)F)cc1)n1c(=O)ccc2cnc(NC3CCN(S(C)(=O)=O)CC3)nc21 ; c1n[nH]c(C2CC2)n1 ; CC(C(=O)Nc1ccc(S(=O)(=O)n2cnc(C3CC3)n2)cc1)n1c(=O)ccc2cnc(NC3CCN(S(C)(=O)=O)CC3)nc21]

### Step 1

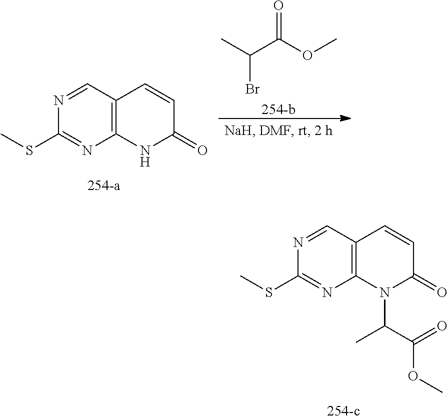

To a solution of 254-a (500 mg, 2.58 mmol) in dry DMF (15 mL) was added 254-b (0.4 mL, 3.88 mmol) under nitrogen atmosphere at 0° C. After 20 min, NaH (207.0 mg, 5.17 mmol) was added and stirred at rt for 2 h. The reaction was stirred at rt for 1 h. Ice-water was added thereto, the mixture was extracted with ethyl acetate (20 ml×2). The organic layer was washed with brine (20 ml), dried over anhydrous Na₂SO₄, filtered and concentrated in vacuo. The residue was purified by silica gel flash chromatography (gradient 0%-30% EtOAc in hexanes) to afford 254-c (260 mg, 35.97%) as a yellow oil. m/z: [M+H]⁺ Calcd for C₁₂H₁₃N₃O₃S 279.1; Found 280.1.

### Step 2

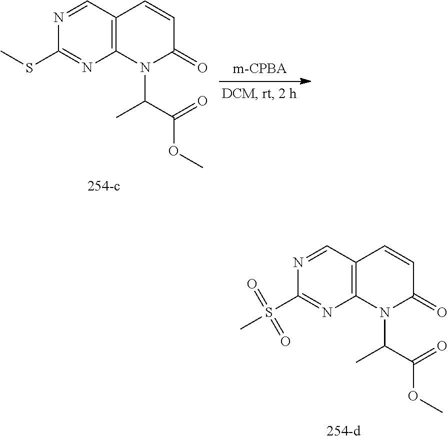 [SMILES: COC(=O)C(C)n1c(=O)ccc2cnc(SC)nc21 ; COC(=O)C(C)n1c(=O)ccc2cnc(S(C)(=O)=O)nc21]

To a solution of 254-c (258 mg, 0.92 mmol) in DCM (15 mL) was added m-CPBA (0.7 mL, 2.31 mmol) at 0° C. The reaction was stirred at rt for 2 h. Then the suspension is filtered and the filtrate was concentered in vacuo. The residue was purified by silica gel flash chromatography (gradient 0%-40% in EtOAc in DCM) to afford 254-d (185 mg, 63.79%) as a white solid. m/z: [M+H]⁺ Calcd for C₁₂H₁₃N₃O₅S 311.1; Found 312.1.

### Step 3

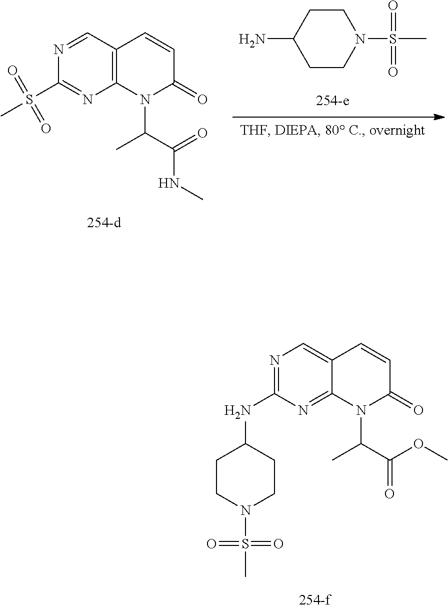

To a solution of 254-d (185 mg, 0.59 mmol) in DCM (10 mL) was added DIPEA (153.6 mg, 1.18 mmol) and 254-e (127.1 mg, 0.71 mmol)). The reaction was stirred at rt overnight. Then additional 254-d (185 mg, 0.59 mmol) was added THF (5 mL) and was stirred at 80° C. overnight. Then the suspension is filtered and the filtra is concentered in vacuo. The residue was purified by silica gel flash chromatography (gradient 0%-10% MeOH in DCM) to afford 254-f (194 mg, 79.73%) as a yellow solid. m/z: [M+H]⁺ Calcd for C₁₇H₂₃N₅O₅S 409.1; Found 410.2.

### Step 4

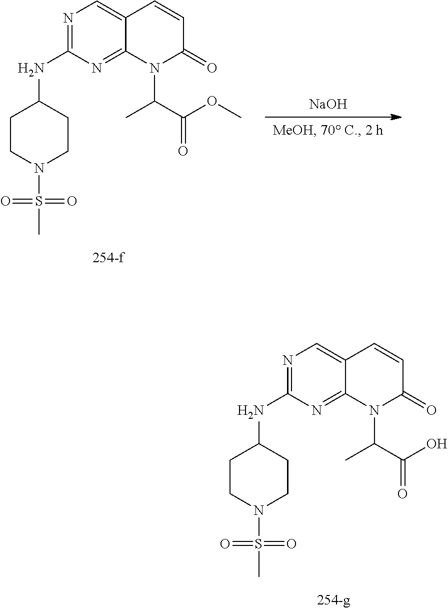 [SMILES: COC(=O)C(C)n1c(=O)ccc2cnc(NC3CCN(S(C)(=O)=O)CC3)nc21 ; "NaOH" ; "MeOH, 70° C., 2 h" ; CC(C(=O)O)n1c(=O)ccc2cnc(NC3CCN(S(C)(=O)=O)CC3)nc21]

To a solution of 254-f (194 mg, 0.474 mmol) in MeOH (10 mL) was added NaOH (94.76 mg, 2.369 mmol). The reaction was stirred at 70° C. for 2 h. Then the suspension is filtered and the filtra is concentered in vacuo. The residue was adjusted PH to 5 with 1 mol/L dilute hydrochloric acid to afford 254-g (89 mg, 0.225 mmol, 47.50%) as a yellow solid. m/z: [M+H]⁺ Calcd for C₁₆H₂₅N₅O₅S 395.1; Found 396.2.

### Step 5

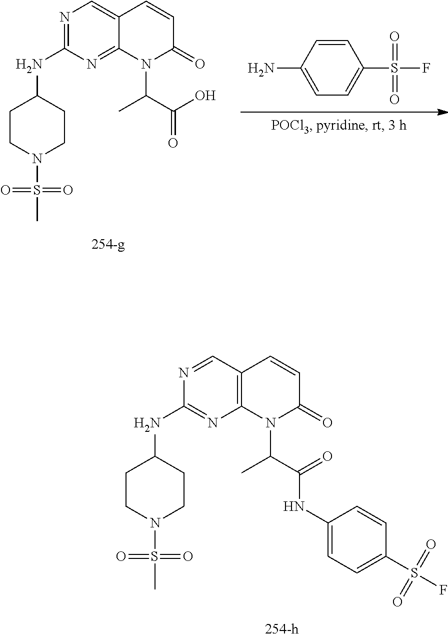

To a solution of 254-g (280 mg, 0.708 mmol) in dry DCM (20 mL) was added POCl3 (0.198 mL, 2.124 mmol) and dry Pyridine (0.286 mL, 3.540 mmol). The reaction was stirred at rt for 3 h. Then the suspension is filtered and the filtra is concentered in vacuo. The residue was purified by reverse phase chromatography (SilaSep™ C18 silica flash cartridge, 40%-60% MeCN in H₂O) to afford 254-h (60 mg, 0.109 mmol, 15.33%) as a white solid. m/z: [M+H]⁺ Calcd for C₂₂H₂₅FN₆O₆S₂552.1; Found 553.0.

### Step 6

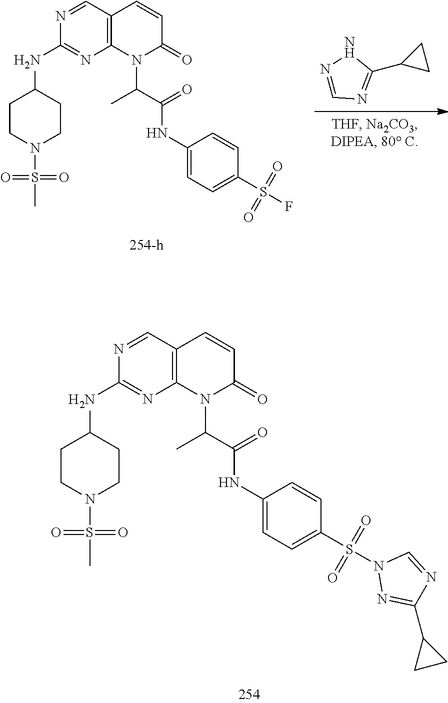

To a solution of 254-h (50 mg, 0.090 mmol) in dry THF (25 mL) was added dry DIPEA (0.030 mL, 0.181 mmol) and Na₂CO₃ (9.59 mg, 0.090 mmol). The reaction was stirred at 80° C. for overnight. Then the suspension is filtered and the filtra is concentered in vacuo. The residue was purified by reverse-phase chromatography (SilaSep™ C18 silica flash cartridge, 40%-60% MeCN in H₂O) to afford 254 (16.5 mg, 0.026 mmol, 28.42%) as a white solid. m/z: [M+H]+ Calcd for C₂₇H₃₁N₉O₆S₂ 641.2; Found 642.5. 1H NMR (400 MHz, DMSO) δ 10.06 (s, 1H), 9.11 (s, 1H), 8.60 (s, 1H), 7.98 (d, J=9.1 Hz, 2H), 7.90-7.74 (m, 4H), 6.33 (d, J=9.3 Hz, 1H), 5.84 (d, J=6.7 Hz, 1H), 3.68-3.37 (m, 3H), 2.92-2.81 (m, 5H), 2.02-1.96 (m, 2H).

### Synthesis of Compound 255

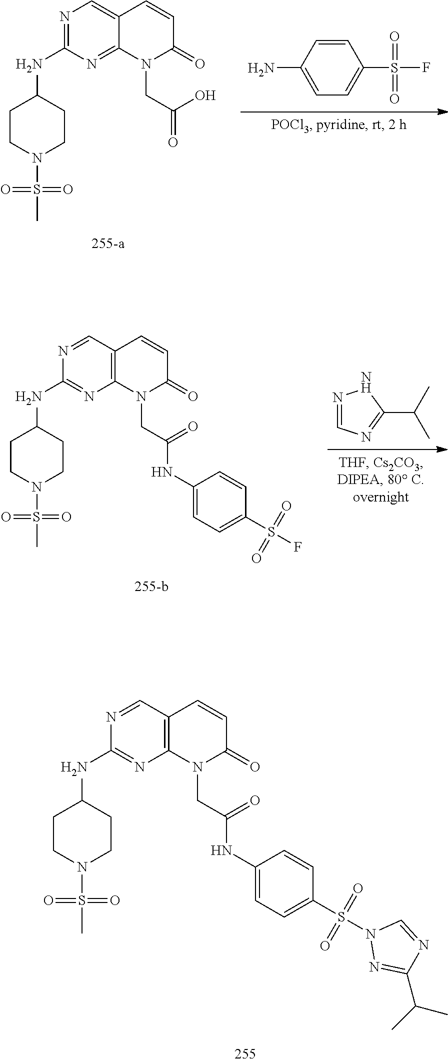

### Step 1

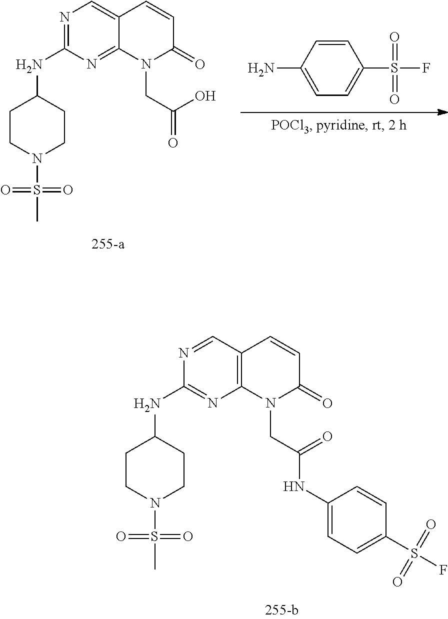

To a solution of 255-a (550 mg, 1.44 mmol) in dry DCM (20 mL) was added POCl₃ (1.1 g, 7.21 mmol) and dry pyridine (0.58 mL, 7.21 mmol). The reaction was stirred at rt for 2 h. Water was added thereto, the mixture was extracted with DCM (15 mL×2). The organic layer dried over anhydrous Na₂SO₄, filtered and concentrated in vacuo. The residue was purified by silica gel flash chromatography (gradient 0%-10% MeOH in DCM) to afford 255-b (280 mg, 36.05%) as a yellow solid. m/z: [M+H]+ Calcd for C₂₁H₂₃FN₆O₆S₂ 538.1; Found 539.1.

### Step 2

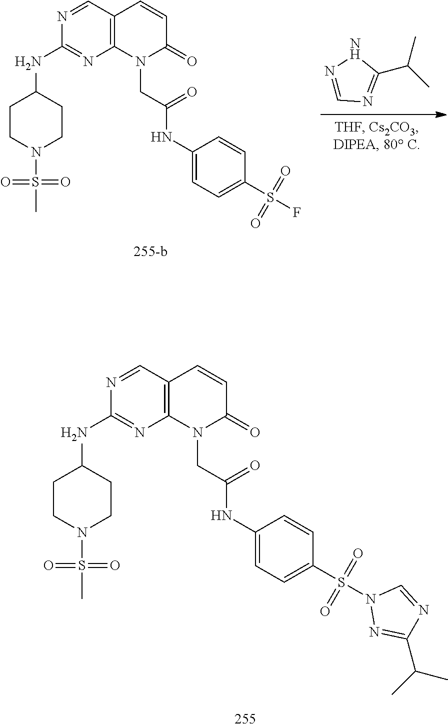

To a solution of 255-b (50 mg, 0.093 mmol) in dry THF (20 mL) was added Cs₂CO₃ (60.60 mg, 0.19 mmol), 3-(propan-2-yl)-1H-1,2,4-triazole (12.38 mg, 0.11 mmol) and DIPEA (0.03 mL, 0.19 mmol). The reaction was stirred at 80° C. for overnight. Then the suspension is filtered and the filtrate is concentered in vacuo. The residue was purified by reverse phase chromatography (SilaSep™ C18 silica lash cartridge, 40%-60% MeCN in H₂O) to afford 255 (7.8 mg, 13.34%) as a white solid. m/z: [M+H]₊ Calcd for C₂₆H₃₁N₉O₆S₂ 629.2; Found 630.4. 1. H NMR (400 MHz, DMSO) δ 11.04 (d, J=23.2 Hz, 1H), 9.13 (s, 1H), 8.66 (d, J=22.7 Hz, 1H), 8.03 (d, J=8.9 Hz, 2H), 7.95-7.76 (m, 4H), 6.33 (d, J=9.5 Hz, 1H), 5.05 (s, 2H), 3.60 (d, J=44.4 Hz, 1H), 3.29-3.25 (m, 2H), 2.78 (d, J=64.9 Hz, 3H), 2.38 (dd, J=22.6, 12.4 Hz, 2H), 2.01 (s, 1H), 1.85 (d, J=50.0 Hz, 2H), 1.42 (d, J=9.1 Hz, 2H), 0.95 (dd, J=8.2, 2.6 Hz, 2H), 0.80 (d, J=2.4 Hz, 2H).

### Synthesis of Compound 256

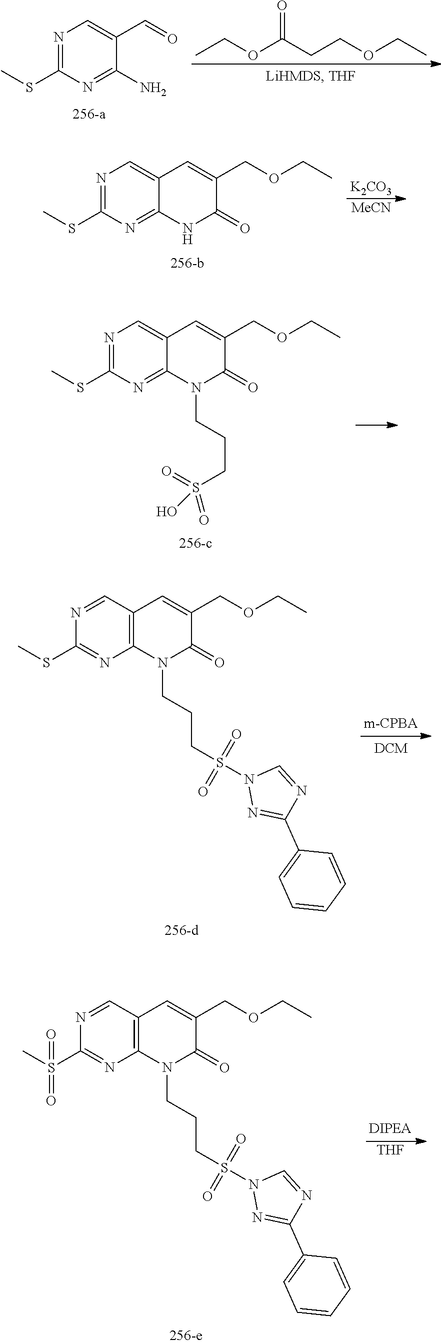

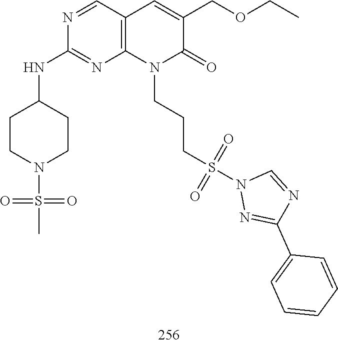 [SMILES: CCOCc1cc2cnc(NC3CCN(S(C)(=O)=O)CC3)nc2n(CCCS(=O)(=O)n2cnc(-c3ccccc3)n2)c1=O]

### Step 1

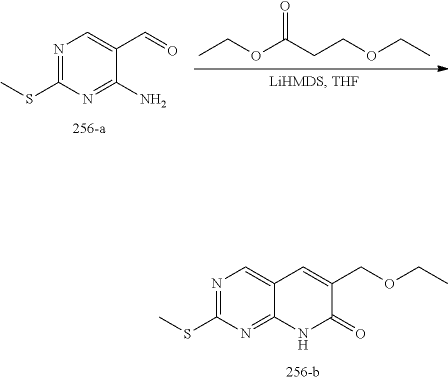

To a solution of ethyl 3-ethoxypropanoate (2.7 mL, 17.79 mmol) in THF (20 mL) was added Lithiumbis(trimethylsilyl)amide (1M solution in THF) (19 mL) with stirring at −70 TC. After 20 min, 256-a (1.0 mL, 8.87 mmol) was added and stirred at rt overnight. The reaction mixture was concentrated and purified by silica gel flash chromatography (gradient 0%-100% EtOAc in hexanes) to afford 256-b (700 mg, 31.42%) as a yellow solid. m/z: [M+H]⁺ Calcd for C₁₁H₁₃N₃O₂S 251.1; Found 252.2.

### Step 2

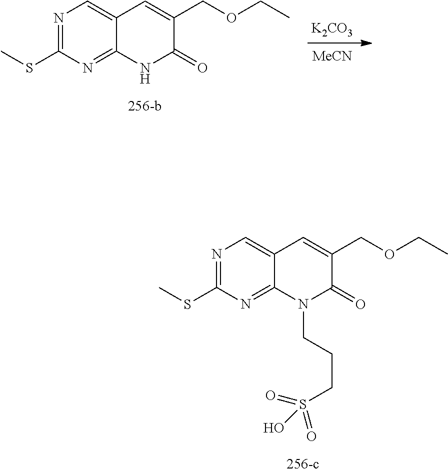

To a solution of 256-b (700 mg, 2.79 mmol) and sodium 3-bromopropane-1-sulfonate (900 mg, 4.00 mmol) in MeCN (20 mL) was added Cs₂CO₃ (1.8 g, 5.53 mmol) with stirring at rt. After stirred at 80° C. for 2 h, the reaction mixture was concentrated and purified by reverse-phase chromatography (SilaSep™ C18 silica flash cartridge, 40%-60% MeOH in H₂O with 0.1% formic acid) to afford 256-c (850 mg, 81.71%) as a yellow solid. m/z: [M+H]⁺ Calcd for C₁₄H₁₉N₃O₅S₂ 373.1; Found 373.8.

### Step 3

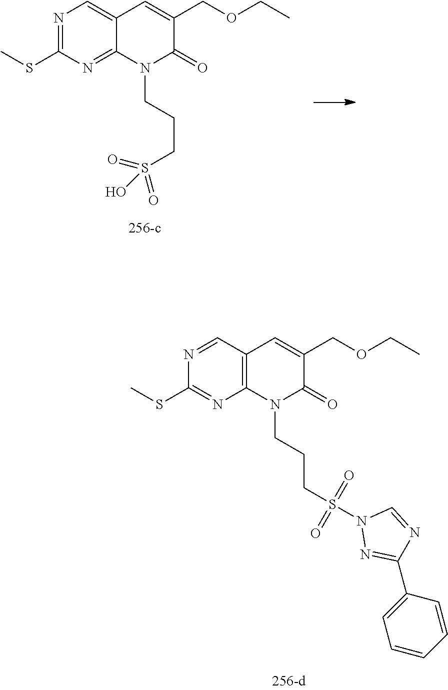

To a solution of 256-c (100 mg, 0.27 mmol) in THF (10 mL) and DCM (10 mL) was added oxalyl chloride (68 mg, 0.54 mmol) with stirring at rt. After 1 h, the reaction mixture was concentrated. The residue was added to the solution of 3-phenyl-1H-1,2,4-triazole (50 mg, 0.34 mmol) and DIEA (70 mg, 0.54 mmol) in DCM (10 mL). After 1 h, the reaction mixture was concentrated and purified by preparative thin-layer chromatography (50% EtOAc in hexanes) to afford 256-d (60 mg, 44.76%) as a white solid. m/z: [M+H]⁺ Calcd for C₂₂H₂₄N₆O₄S₂ 500.1; Found 500.8.

### Step 4

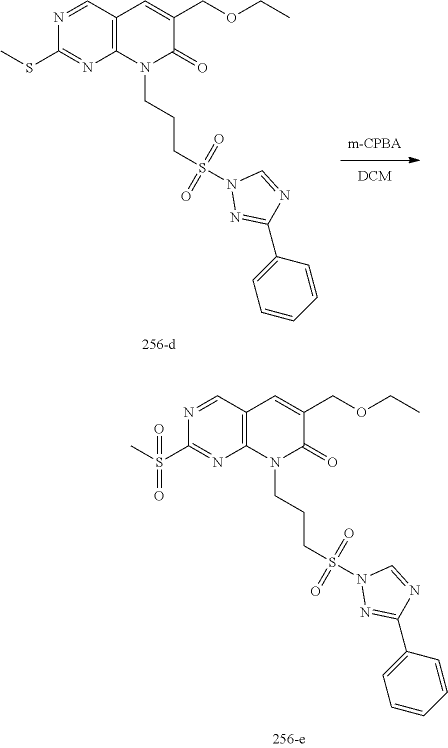

To a solution of 256-d (60 mg, 0.12 mmol) in DCM (10 mL) was added m-CPBA (70 mg, 0.41 mmol) with stirring at rt. After stirring at rt for 1 h, the reaction mixture was concentrated and purified by silica gel flash chromatography (gradient 0%-100% EtOAc in hexanes) to afford 256-e (60 mg, 93.99%) as a yellow solid. m/z: [M+H]⁺ Calcd for C₂₂H₂₄N₆O₆S₂ 532.1; Found 532.7.

### Step 5

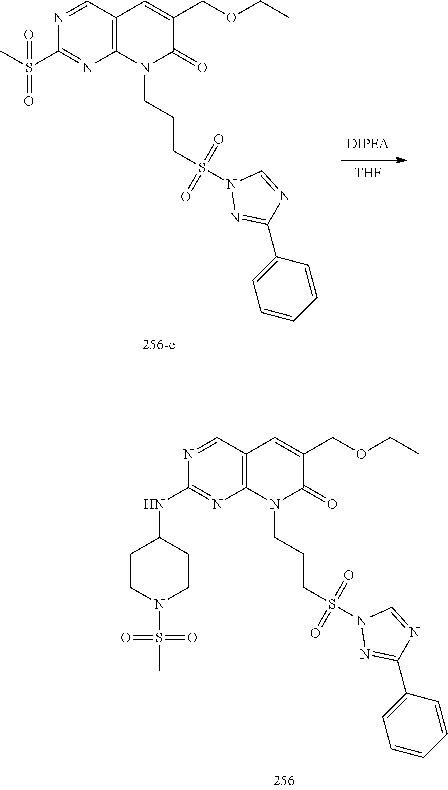 [SMILES: CCOCc1cc2cnc(S(C)(=O)=O)nc2n(CCCS(=O)(=O)n2cnc(-c3ccccc3)n2)c1=O ; CCOCc1cc2cnc(NC3CCN(S(C)(=O)=O)CC3)nc2n(CCCS(=O)(=O)n2cnc(-c3ccccc3)n2)c1=O ; "THF"]

To a solution of 256-e (60 mg, 0.11 mmol) and DIEA (50 mg, 0.39 mmol) in THF (10 mL) was added 1-methanesulfonylpiperidin-4-amine (40 mg, 0.22 mmol) with stirring at rt. After stirred at 80° C. for 1 h, the reaction mixture was concentrated and purified by reverse-phase chromatography (SilaSep™ C18 silica flash cartridge, 50%-70% MeCN in H₂O with 0.1% formic acid) to 256 (5 mg, 7.04%) as a white solid. m/z: [M+H]⁺ Calcd for C₂₇H₃₄N₈O₆S₂ 630.2; Found 631.1. ¹H NMR (400 MHz, DMSO) δ 9.24 (s, 1H), 8.58 (s, 1H), 8.05-7.85 (m, 3H), 7.62 (s, 1H), 7.55-7.50 (m, 3H), 4.34 (m, 2H), 4.27 (s, 2H), 4.00 (m, 3H), 3.58-3.52 (m, 4H), 2.95-2.86 (m, 5H), 2.09 (s, 2H), 1.93 (m, 2H), 1.55 (m, 2H), 1.18 (t, J=7.0 Hz, 3H)

### Synthesis of Compound 258

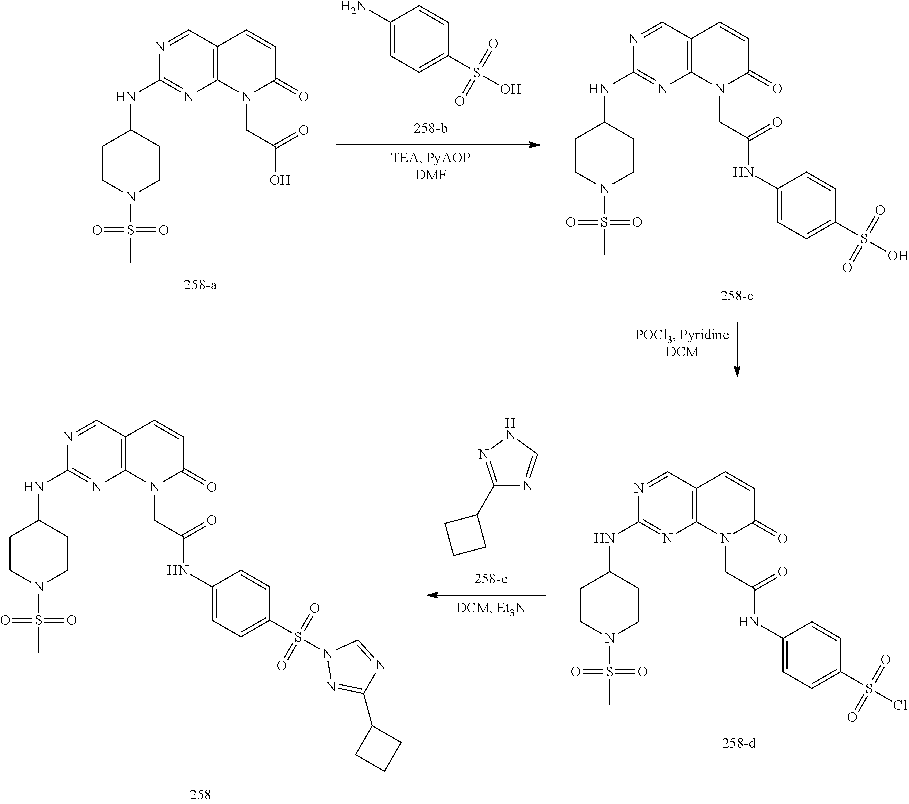

### Step 1

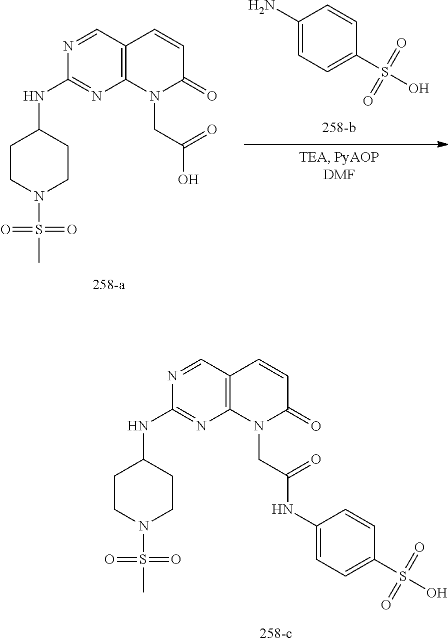 [SMILES: CS(=O)(=O)N1CCC(Nc2ncc3ccc(=O)n(CC(=O)O)c3n2)CC1 ; Nc1ccc(S(=O)(=O)O)cc1 ; "TEA, PyAOPDMF" ; CS(=O)(=O)N1CCC(Nc2ncc3ccc(=O)n(CC(=O)Nc4ccc(S(=O)(=O)O)cc4)c3n2)CC1]

To a stirred solution of 258-a (800 mg, 2.10 mmol) and 258-b (0.25 mL, 2.10 mmol) in DMF (10 mL) was added TEA (0.44 mL, 3.15 mmol) and PyAOP (1.2 g, 2.31 mmol) under N2. After stirring at rt for 2 h. The reaction mixture was concentrated was purified by silica gel flash chromatography (gradient 0%-40% MeOH in DCM) to afford 258-c (1.08 g, 95.96%) as a yellow solid. LC/MS (ESI) m/z: 537 (M+H)⁺.

### Step 2

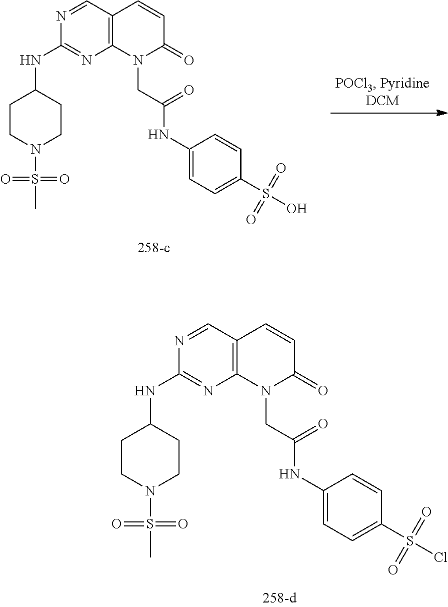 [SMILES: CS(=O)(=O)N1CCC(Nc2ncc3ccc(=O)n(CC(=O)Nc4ccc(S(=O)(=O)O)cc4)c3n2)CC1 ; "POCl3, PyridineDCM" ; CS(=O)(=O)N1CCC(Nc2ncc3ccc(=O)n(CC(=O)Nc4ccc(S(=O)(=O)Cl)cc4)c3n2)CC1]

To a stirred solution of 258-c (1.08 g, 2.01 mmol) and pyridine (0.25 mL, 3.02 mmol) in DCM (10 mL) was added POCl₃ (0.6 mL, 6.04 mmol) under N2. After stirring at rt for 2 h. The reaction mixture was concentrated and residue was extract with DCM to afford 258-d (150 mg, 13.43%) as a yellow solid. LC/MS(ESI) m/z: 555 (M+H)⁺.

### Step 3

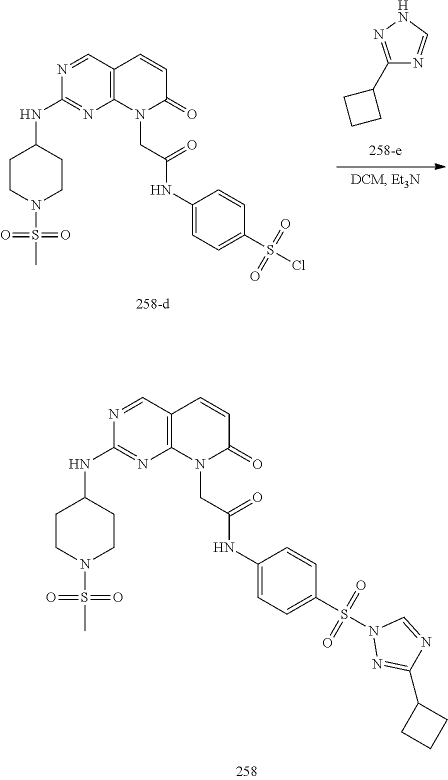

To a stirred solution of 258-d (100 mg, 0.18 mmol) and 258-e in DCM (5 mL) was added TEA (0.03 mL, 0.18 mmol) under N2. After stirring at rt for 2 h. The reaction mixture was purified by reverse-phase chromatography (SilaSep™ C18 silica flash cartridge, 40%-60% MeCN in H₂O with 0.1% formic acid) to afford 258 (7.5 mg, 6.49%) as a white solid. LC/MS (ESI) m/z: 642 (M+H)⁺. ¹H NMR (400 MHz, DMSO) δ 11.04 (m, J=24.6 Hz, 1H), 9.21 (s, 1H), 8.66 (m, J=22.4 Hz, 1H), 8.04 (d, J=8.9 Hz, 2H), 7.95-7.70 (m, 4H), 6.33 (d, J=9.5 Hz, 1H), 5.05 (s, 2H), 3.58 (m, 2H), 3.27 (m, 2H), 2.77 (m, 3H), 2.39 (m, 2H), 2.33-2.15 (m, 4H), 2.01-1.86 (m, 2H), 1.79 (m, 2H), 1.45 (m, 2H).

### Synthesis of Compound 261

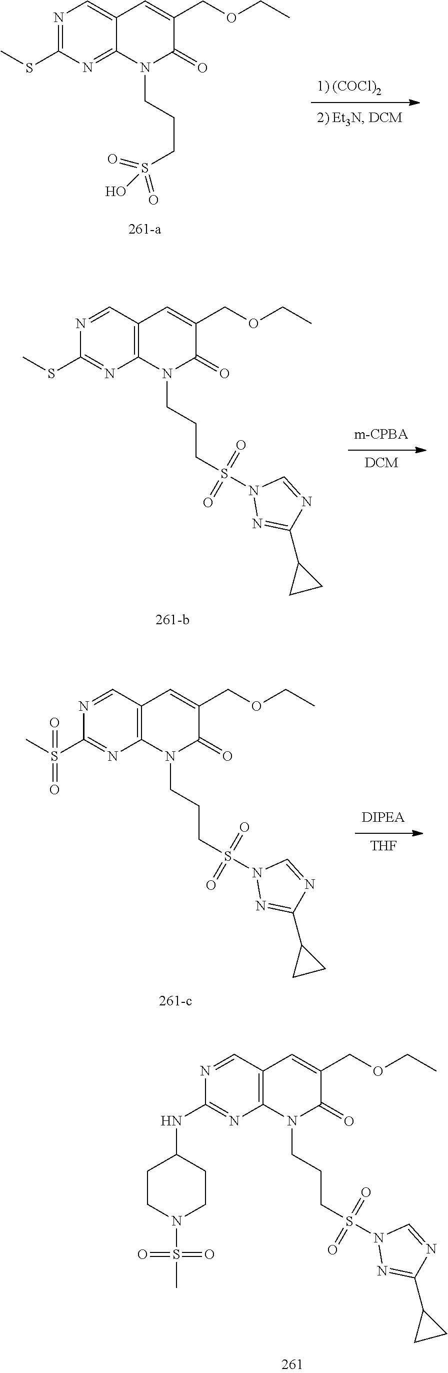

### Step 1

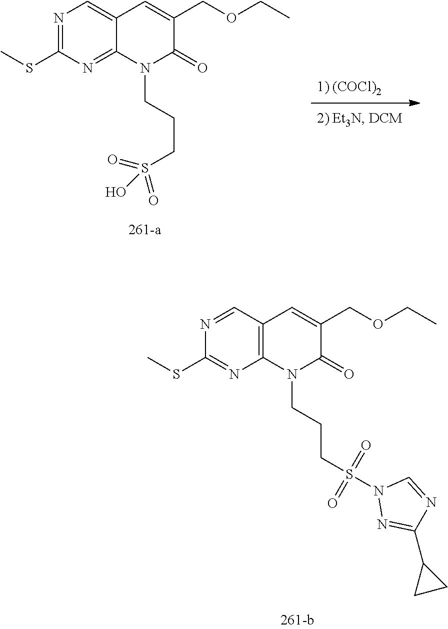

To a solution of 261-a (150 mg, 0.40 mmol) and 10 drops of DMF in DCM (10 mL) and THF (10 mL) was added oxalyl chloride (110 mg, 0.87 mmol) at 0° C. Then stirred at rt for 1 h. with stirring at rt, the mixture was concentrated. The solid was added to 3-cyclopropyl-1H-1,2,4-triazole (50 mg, 0.46 mmol) and Et₃N (100 mg, 0.99 mmol) in DCM (10 mL). After stirring at rt for 1 h, the reaction mixture was concentrated and purified by silica gel flash chromatography (gradient 0%-100% EtOAc in hexanes) to afford 261-b (60 mg, 32.16%) as a yellow solid. m/z: [M+H]⁺ Calcd for C₁₉H₂₄N₆O₄S₂ 464.1; Found 464.8.

### Step 2

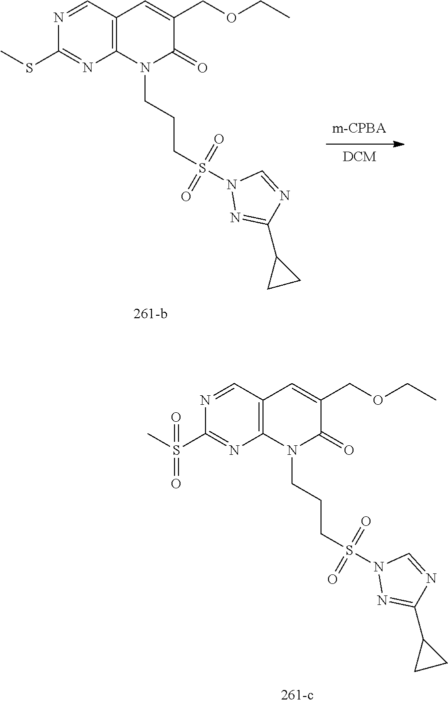

To a solution of 261-b (60 mg, 0.13 mmol) in DCM (10 mL) was added m-CPBA (0.04 mL, 0.13 mmol) with stirring at rt. After stirring at rt for 1 h, the reaction mixture was concentrated and purified by silica gel flash chromatography (gradient 0%-100% EtOAc in hexanes) to afford 261-c (60 mg, 93.56%) as a yellow solid. m/z: [M+H]⁺ Calcd for C₁₉H₂₄N₆O₆S₂ 496.1; Found 496.7.

### Step 3

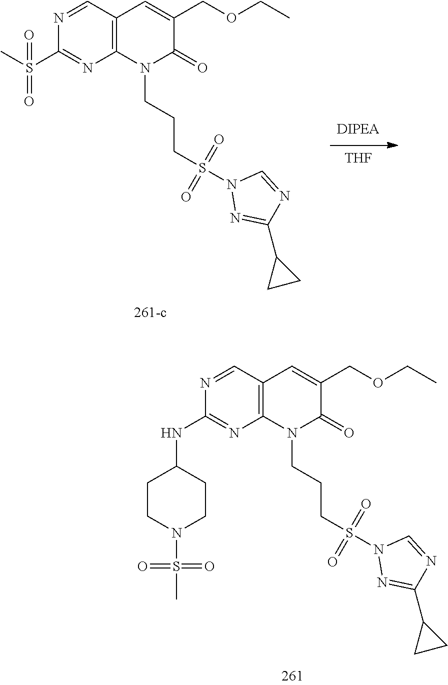 [SMILES: CCOCc1cc2cnc(S(C)(=O)=O)nc2n(CCCS(=O)(=O)n2cnc(C3CC3)n2)c1=O ; "THF" ; CCOCc1cc2cnc(NC3CCN(S(C)(=O)=O)CC3)nc2n(CCCS(=O)(=O)n2cnc(C3CC3)n2)c1=O]

To a solution of 261-c (60 mg, 0.12 mmol) and DIEA (60 mg, 0.46 mmol) in THF (10 mL) was added 1-methanesulfonylpiperidin-4-amine (40 mg, 0.22 mmol) with stirring at rt. After stirred at 80° C. for 1 h, the reaction mixture was concentrated and purified by reverse-phase chromatography (SilaSep™ C18 silica flash cartridge, 50%-70% MeCN in H₂O with 0.1% formic acid) to afford 261 (26.7 mg, 37.16%) as a white solid. m/z: [M+H]⁺ Calcd for C₂₄H₃₄N₈O₆S₂ 594.2; Found 595.5. ¹H NMR (400 MHz, DMSO) δ 8.96 (s, 1H), 8.67 (s, 1H), 7.95-7.62 (m, 2H), 4.32 (d, J=11.6 Hz, 4H), 3.93 (m, 3H), 3.57 (q, J=7.0 Hz, 4H), 2.97-2.86 (m, 5H), 2.07-1.93 (m, 5H), 1.59 (m, 2H), 1.20 (t, J=7.0 Hz, 3H), 1.03-0.97 (m, 2H), 0.93-0.77 (m, 2H)

### Synthesis of Compound 264

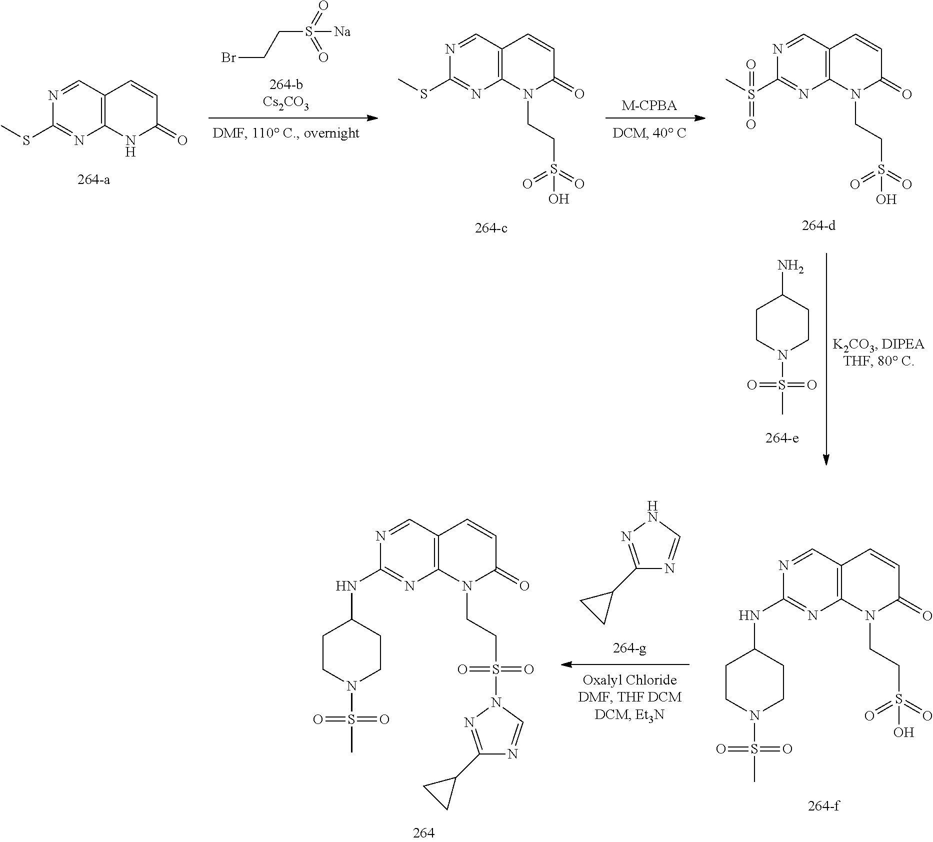

### Step 1

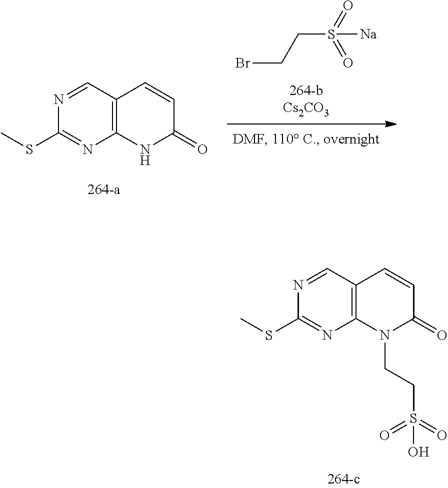

To a solution of 264-a (0.65 g, 3.36 mmol) and 264-b (0.64 g, 3.36 mmol) in DMF (20 mL) was added Cs₂CO₃ (2.19 g, 6.73 mmol) with stirring at rt. After stirred at 110° C. overnight, the reaction mixture was concentrated and purified by reverse-phase chromatography (SilaSep™ C18 silica flash cartridge, 40%-60% MeOH in H₂O with 0.1% formic acid) to afford 264-c (0.75 g, 73.99%) as a yellow solid. LC/MS (ESI) m/z: 302 (M+H)⁺.

### Step 2

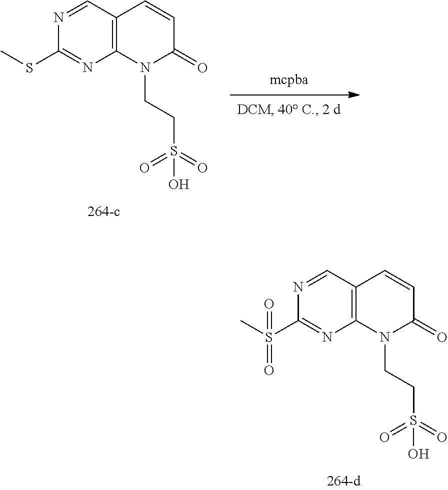 [SMILES: CSc1ncc2ccc(=O)n(CCS(=O)(=O)O)c2n1 ; CS(=O)(=O)c1ncc2ccc(=O)n(CCS(=O)(=O)O)c2n1]

To a stirred solution of 264-c (0.75 g, 2.49 mmol) and m-CPBA (7.67 g, 24.89 mmol) in DCM (20 mL) under N2. After stirring at 40° C. for 2 days. The reaction mixture was concentrated and washed with water to afford 264-d (500 mg, 60.27%) as a yellow solid. LC/MS (ESI) m/z: 332 (M−H)⁻.

### Step 3

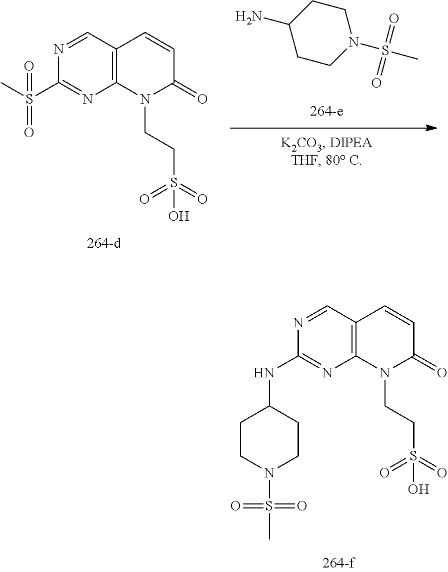 [SMILES: CS(=O)(=O)c1ncc2ccc(=O)n(CCS(=O)(=O)O)c2n1 ; CS(=O)(=O)N1CCC(N)CC1 ; CS(=O)(=O)N1CCC(Nc2ncc3ccc(=O)n(CCS(=O)(=O)O)c3n2)CC1]

To a stirred solution of 264-d (800 mg, 2.40 mmol) and 264-e (427.8 mg, 2.40 mmol) in THF (40 mL) was added DIPEA (0.8 mL, 4.80 mmol) and K2CO₃ (331.7 mg, 2.40 mmol) under N2. After stirring at 80° C. for 3 days. The reaction mixture was purified by reverse-phase chromatography (SilaSep™ C18 silica flash cartridge, 0%-60% MeCN in H₂O with 0.1% formic acid) to afford 264-f (430 mg, 41.52%) as a white solid. LC/MS (ESI) m/z: 432 (M+H)⁺.

### Step 4

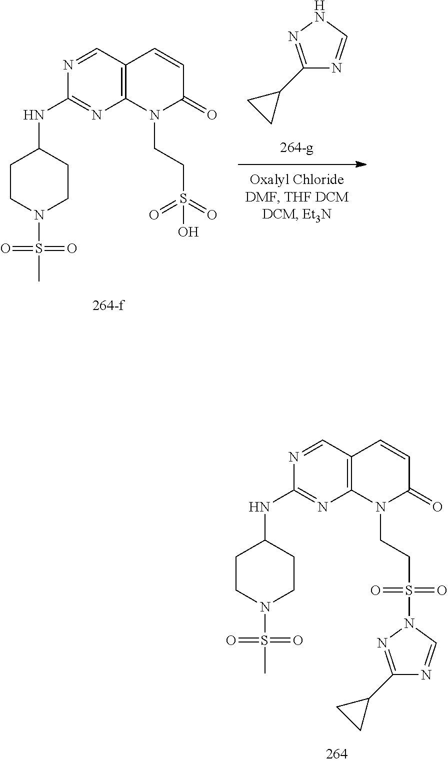

To a solution of 264-f (90 mg, 0.21 mmol) and 10 drops of DMF in THF (5 mL) and DCM (5 mL) was added oxalyl chloride (0.04 mL, 0.42 mmol) at 0° C. Then stirred at rt for 1 h, the mixture was concentrated. The solid was added to 264-g (34.1 mg, 0.31 mmol) and TEA (0.04 mL, 0.31 mmol) in DCM (7 mL). After stirring at rt for 1 h, the reaction mixture was concentrated and purified by silica gel flash chromatography (gradient 50%-100% EtOAc in hexanes) to afford 264 (4.4 mg, 4.04%) as a white solid. LC/MS (ESI) m/z: 523 (M+H)⁺. ¹H NMR (400 MHz, DMSO) δ 8.84 (s, 1H), 8.60 (s, 1H), 8.10-8.00 (m, 1H), 7.72 (d, J=9.7 Hz, 1H), 6.22 (d, J=9.3 Hz, 1H), 4.61 (m, 2H), 4.26 (m, 2H), 3.99 (m, 1H), 3.58 (m, 2H), 2.89 (m, 5H), 1.99 (m, 3H), 1.60 (m, 2H), 1.03-0.96 (m, 2H), 0.80 (m, 2H).

### Synthesis of Compound 265

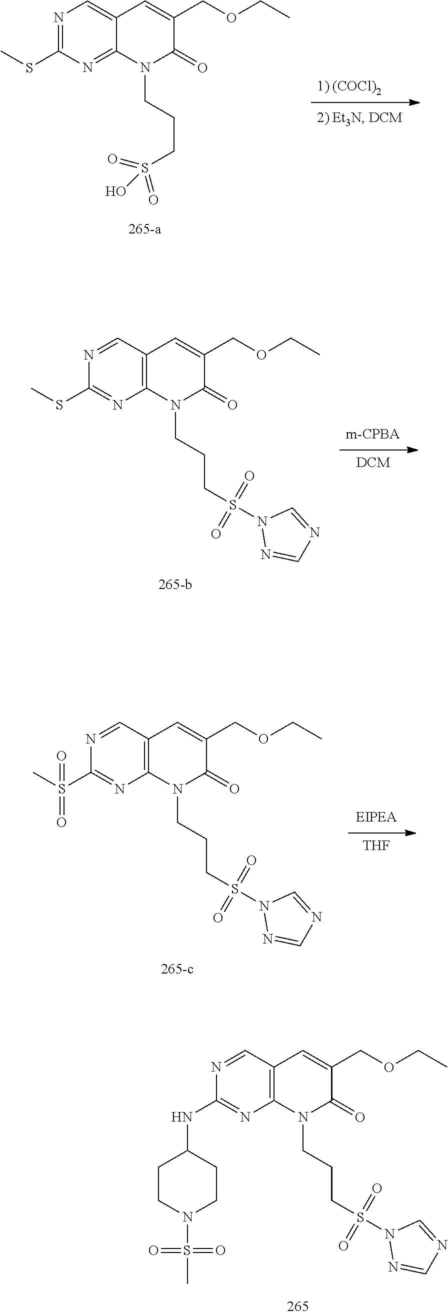

### Step 1

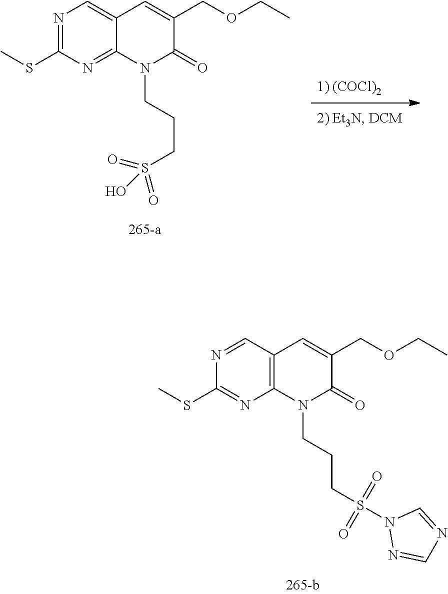

To a solution of 265-a (250 mg, 0.69 mmol) and 10 drops of DMF in DCM (10 mL) and THF (10 mL) was added oxalyl chloride (170 mg, 1.34 mmol) at 0° C. Then stirred at rt for 1 h. After stirring at rt, the mixture was concentrated. The solid was added to 1H-1,2,4-triazole (0.070 mL, 1.16 mmol) and Et₃N (150 mg, 1.49 mmol) in DCM (10 mL). After stirring at rt for 1 h, the reaction mixture was concentrated and purified by silica gel flash chromatography (gradient 0%-100% EtOAc in hexanes) to afford 265-b (140 mg, 49.27%) as a yellow solid. m/z: [M+H]⁺ Calcd for C₁₆H₂₀N₆O₄S₂ 424. 1; Found 424.8.

### Step 2

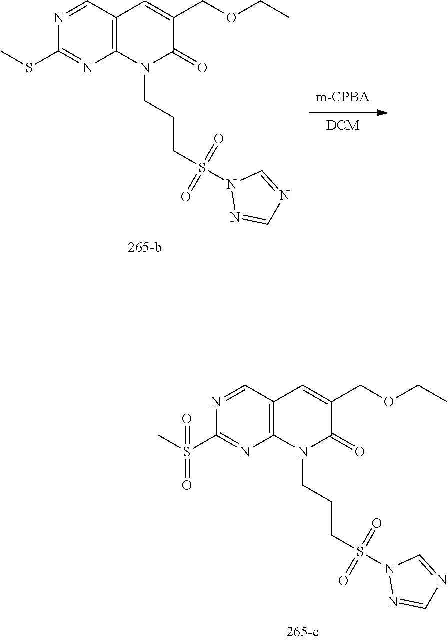

To a solution of 265-b (140 mg, 0.33 mmol) in DCM (10 mL) was added m-CPBA (180 mg, 1.04 mmol) with stirring at rt. After stirring at rt for 1 h, the reaction mixture was concentrated and purified by silica gel flash chromatography (gradient 0%-100% EtOAc in hexanes) to afford 265-c (140 mg, 92.99%) as a yellow solid. m/z: [M+H]⁺ Calcd for C₁₆H₂₀N₆O₆S₂ 456.1; Found 456.8.

### Step 3

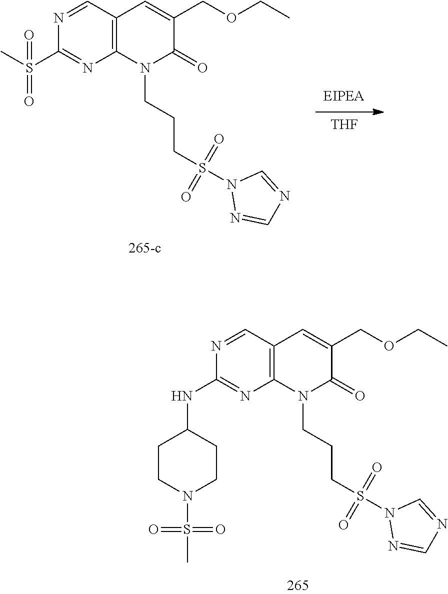 [SMILES: CCOCc1cc2cnc(S(C)(=O)=O)nc2n(CCCS(=O)(=O)n2cncn2)c1=O ; "THF" ; CCOCc1cc2cnc(NC3CCN(S(C)(=O)=O)CC3)nc2n(CCCS(=O)(=O)n2cncn2)c1=O]

To a solution of 265-c (140 mg, 0.31 mmol) and DIEA (100 mg, 0.77 mmol) in THF (10 mL) was added 1-methanesulfonylpiperidin-4-amine (100 mg, 0.56 mmol) with stirring at rt. After stirred at 80° C. for 1 h, the reaction mixture was concentrated and purified by reverse-phase chromatography (SilaSep™ C18 silica flash cartridge, 40%-60% MeCN in H₂O with 0.1% formic acid) to afford 265 (18.4 mg, 10.82%) as a white solid. m/z: [M+H]⁺ Calcd for C₂₁H₃₀N₈O₆S₂ 554.2; Found 555.0. ¹H NMR (400 MHz, DMSO) δ 9.20 (s, 1H), 8.67 (m, 1H), 8.40 (s, 1H), 7.93-7.63 (m, 2H), 4.34-4.24 (m, 4H), 4.04-3.91 (m, 3H), 3.56 (q, J=7.0 Hz, 4H), 2.97-2.87 (m, 5H), 1.98 (s, 4H), 1.62-1.53 (m, 2H), 1.19 (t, J=7.0 Hz, 3H)

### Synthesis of Compound 268

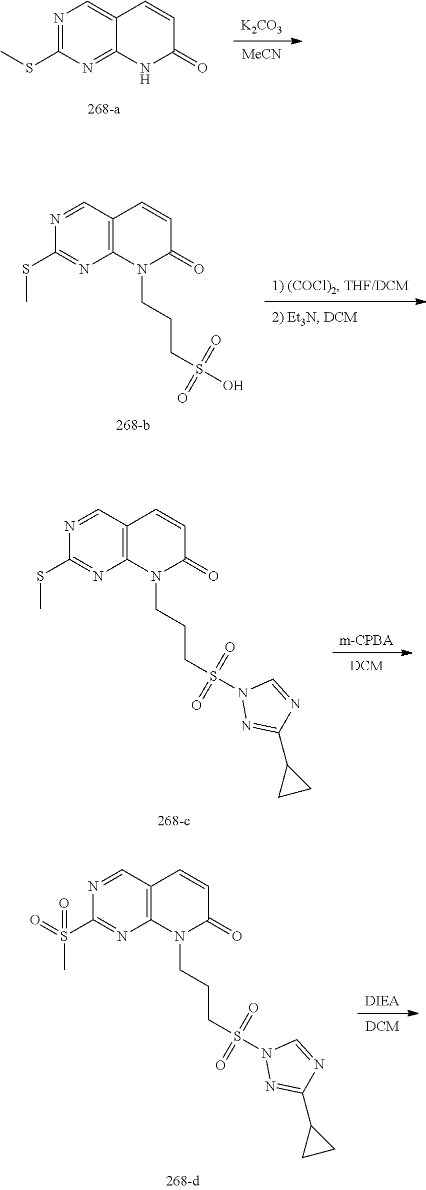

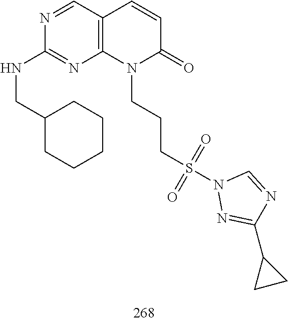 [SMILES: O=c1ccc2cnc(NCC3CCCCC3)nc2n1CCCS(=O)(=O)n1cnc(C2CC2)n1]

### Step 1

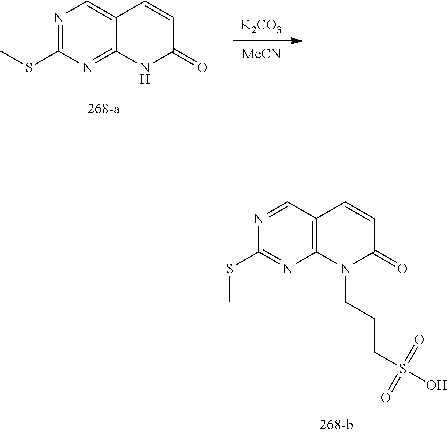

To a solution of 268-a (1 g, 5.18 mmol) and sodium 3-bromopropane-1-sulfonate (1.6 g, 7.11 mmol) in MeCN (30 mL) was added Cs₂CO₃ (3.3 g, 10.13 mmol) with stirring at rt. After stirred at 80° C. for 2 h, the reaction mixture was concentrated and purified by reverse-phase chromatography (SilaSep™ C18 silica flash cartridge, 40%-60% MeOH in H₂O with 0.1% formic acid) to afford 268-b (1.45 g, 88.84%) as a yellow solid. m/z: [M+H]⁺ Calcd for C₁₁H₁₃N₃O₄S₂ 315.0; Found 315.8.

### Step 2

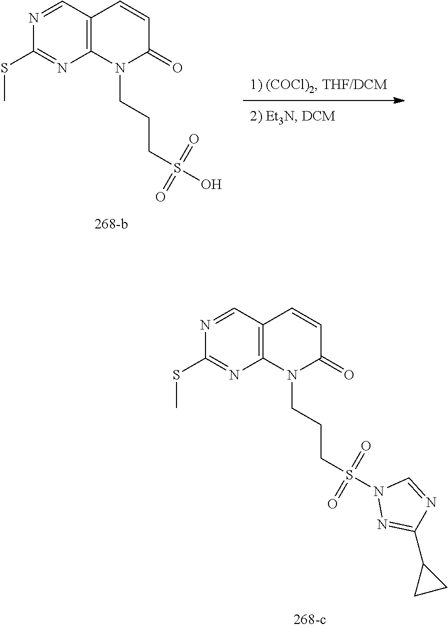 [SMILES: CSc1ncc2ccc(=O)n(CCCS(=O)(=O)O)c2n1 ; CSc1ncc2ccc(=O)n(CCCS(=O)(=O)n3cnc(C4CC4)n3)c2n1]

To a solution of 268-b (2.8 g, 8.88 mmol) and 10 drops of DMF in DCM (30 mL) and THF (30 mL) was added oxalyl chloride (1.7 mL, 19.69 mmol) at 0° C. Then stirred at rt for 1 h. After stirring at rt, the mixture was concentrated. The solid was added to 3-cyclopropyl-1H-1,2,4-triazole (1.2 g, 10.99 mmol) and Et₃N (2 g, 19.80 mmol) in DCM (30 mL). After stirring at rt for 1 h, the reaction mixture was concentrated and purified by silica gel flash chromatography (gradient 0%-100% EtOAc in hexanes) to afford 268-c (1.2 g, 33.25%) as a white solid. m/z: [M+H]⁺ Calcd for C₁₆H₁₈N₆O₃S₂ 406.1; Found 406.8.

### Step 3

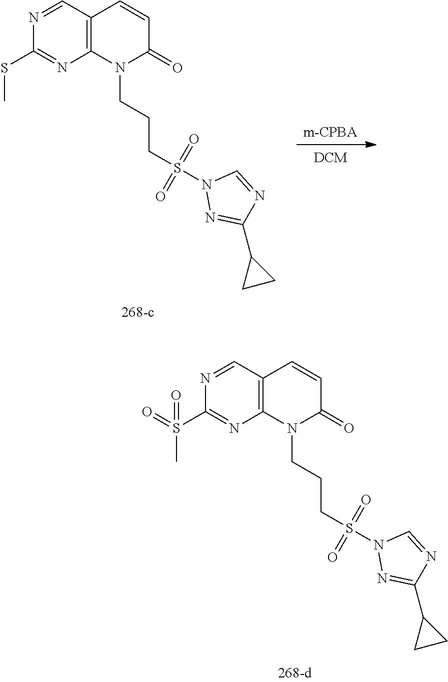

To a solution of 268-c (1.2 g, 2.95 mmol) in DCM (60 mL) was added m-CPBA (1.7 g, 9.85 mmol) with stirring at rt. After stirring at rt for 1 h, the reaction mixture was concentrated and purified by silica gel flash chromatography (gradient 0%-50% EtOAc in hexanes) to afford 268-d (1.2 g, 92.70%) as a white solid. m/z: [M+H]⁺ Calcd for C₁₆H₁₈N₆O₅S₂ 438.1; Found 438.7.

### Step 4

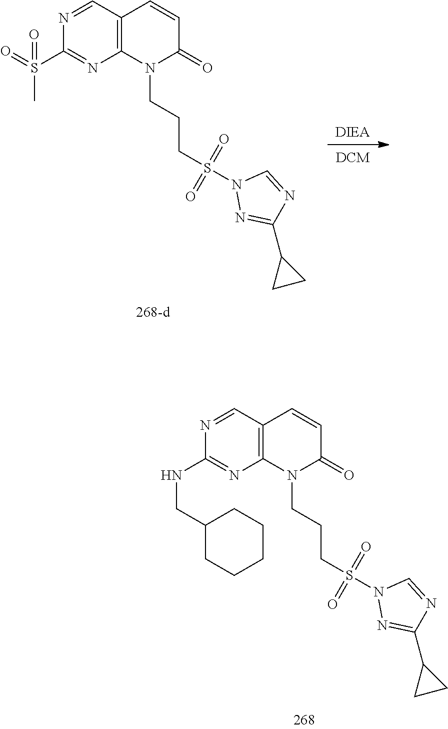

To a solution of 268-d (70 mg, 0.16 mmol) and DIEA (60 mg, 0.46 mmol) in DCM (10 mL) was added cyclohexylmethanamine (0.02 mL, 0.17 mmol) with stirring at 0° C. After stirred at rt for 1 h, the reaction mixture was concentrated and purified by reverse-phase chromatography (SilaSep™ C18 silica flash cartridge, 40%-60% MeCN in H₂O with 0.1% formic acid) to afford 268 (37 mg, 49.15%) as a white solid. m/z: [M+H]⁺ Calcd for C₂₂H₂₉N₇O₃S 471.2; Found 472.5. ¹H NMR (400 MHz, DMSO) δ 8.95 m, 1H), 8.58 (m, 1H), 7.95 (t, J=6.0 Hz, 1H), 7.72 (d, J=9.3 Hz, 1H), 6.22 (d, J=9.3 Hz, 1H), 4.28 (m, 2H), 3.95-3.78 (m, 2H), 3.19 (m, 2H), 2.06-1.90 (m, 3H), 1.79-1.66 (m, 4H), 1.64-1.54 (m, 2H), 1.22-0.82 (m, 9H).

### Synthesis of Compound 274

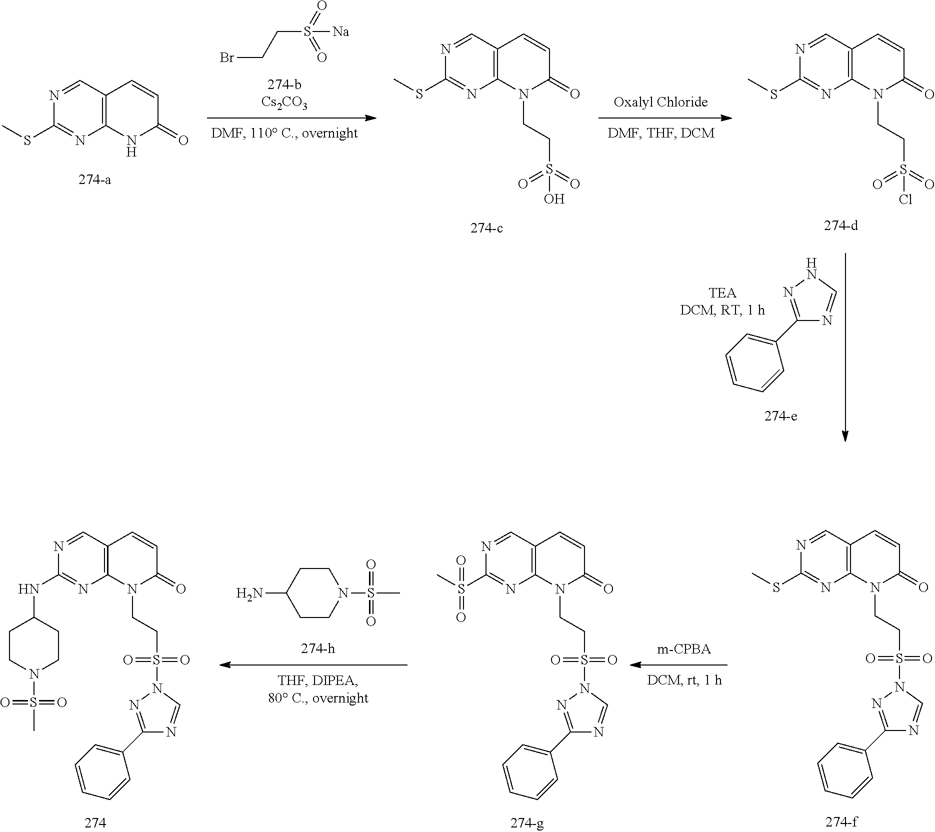

### Step 1

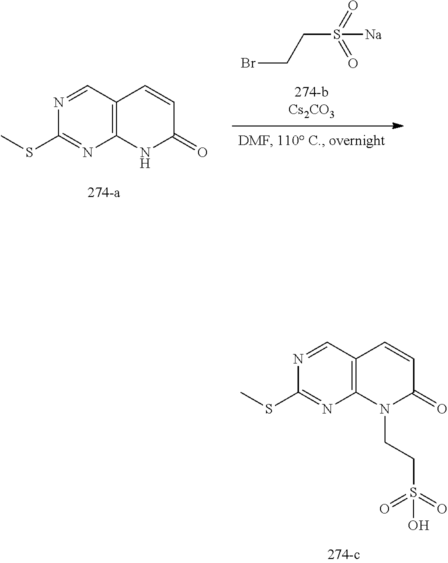

To a solution of 274-a (650 mg, 3.36 mmol) and 274-b (699.4 mg, 3.70 mmol) in DMF (20 mL) was added Cs₂CO₃ (1.64 g, 5.05 mmol) with stirring at rt. After stirred at 110° C. overnight, the reaction mixture was concentrated and purified by reverse-phase chromatography (SilaSep™ C18 silica flash cartridge, 40%-60% MeOH in H₂O with 0.1% formic acid) to afford 274-c (650 mg, 64.12%) as a yellow solid. LC/MS (ESI) m/z: 302 (M+H)⁺.

### Step 2

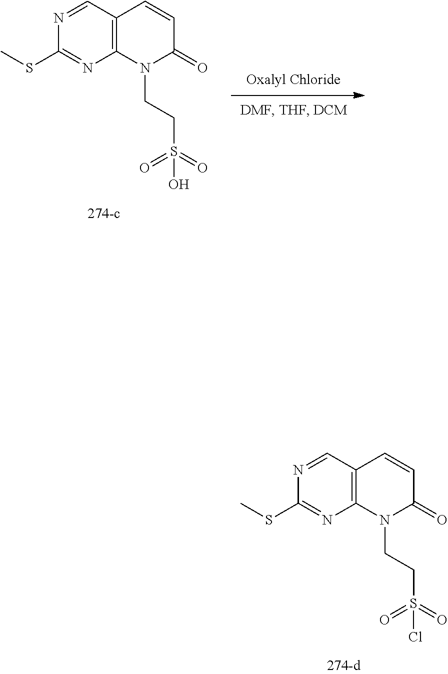 [SMILES: CSc1ncc2ccc(=O)n(CCS(=O)(=O)O)c2n1 ; CSc1ncc2ccc(=O)n(CCS(=O)(=O)Cl)c2n1]

To a stirred solution of 274-c (200 mg, 0.66 mmol) in THF (5 mL) and DCM (5 mL) and DMF (0.2 mL) was added oxalyl chloride (0.1 mL, 1.33 mmol) under N2. After stirring at rt for 2 h, the reaction mixture was concentrated to afford 274-d (35 mg, 16.51%) as a yellow solid. LC/MS (ESI) m/z: 319.9 (M+H)⁺.

### Step 3

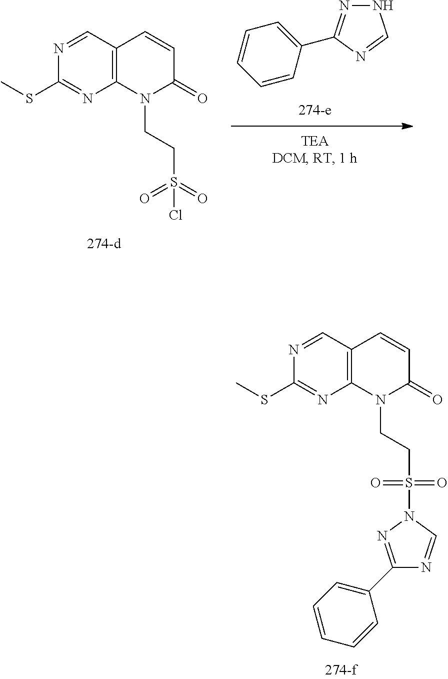 [SMILES: CSc1ncc2ccc(=O)n(CCS(=O)(=O)Cl)c2n1 ; c1ccc(-c2nc[nH]n2)cc1 ; CSc1ncc2ccc(=O)n(CCS(=O)(=O)n3cnc(-c4ccccc4)n3)c2n1]

To a stirred solution of 274-d (35 mg, 0.11 mmol) and 274-e (19.1 mg, 0.13 mmol) in DCM (7 mL) was added TEA (0.03 mL, 0.22 mmol) under N2. After stirring at rt for 1 h. The reaction mixture was purified by silica gel flash chromatography (gradient 0%-10% MeOH in DCM) to afford 274-f (40 mg, 85.29%) as a yellow solid. LC/MS (ESI) m/z: 429 (M+H)⁺.

### Step 4

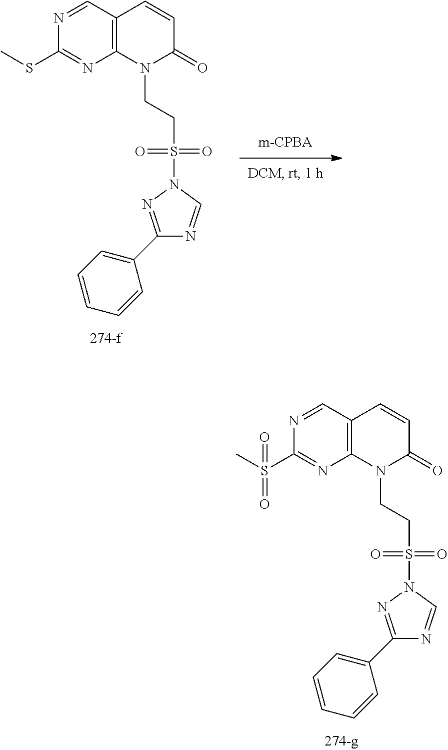 [SMILES: CSc1ncc2ccc(=O)n(CCS(=O)(=O)n3cnc(-c4ccccc4)n3)c2n1 ; CS(=O)(=O)c1ncc2ccc(=O)n(CCS(=O)(=O)n3cnc(-c4ccccc4)n3)c2n1]

To a stirred solution of 274-f (40 mg, 0.093 mmol) in DCM (7 mL) was added m-CPBA (0.03 mL, 0.093 mmol). After stirring at rt for 1 h, the residue was purified by silica gel flash chromatography (gradient 0%-10% MeOH in DCM) to afford 274-g (40 mg, 93.05%) as a white solid. LC/MS (ESI) m/z: 461 (M+H)⁺.

### Step 5

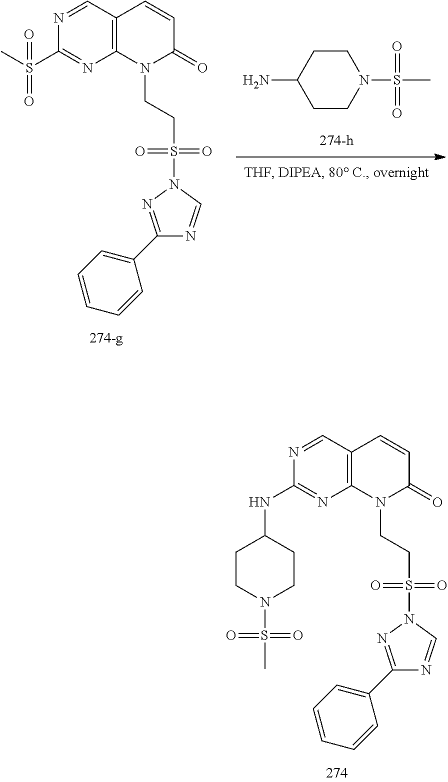 [SMILES: CS(=O)(=O)c1ncc2ccc(=O)n(CCS(=O)(=O)n3cnc(-c4ccccc4)n3)c2n1 ; CS(=O)(=O)N1CCC(N)CC1 ; CS(=O)(=O)N1CCC(Nc2ncc3ccc(=O)n(CCS(=O)(=O)n4cnc(-c5ccccc5)n4)c3n2)CC1]

To a stirred solution of 274-g (40 mg, 0.087 mmol) and 274-h (15.5 mg, 0.087 mmol) in THF (10 mL) was added DIPEA (0.03 mL, 0.17 mmol) under N2. After stirring at 80° C. overnight, the reaction mixture was purified by silica gel flash chromatography (gradient 0%-10% MeOH in DCM) to afford 274 (9.9 mg, 20.40%) as a yellow solid. LC/MS (ESI) m/z: 559.1 (M+H)⁺. ¹H NMR (400 MHz, DMSO) δ 9.13 (m, 1H), 8.47 (m, 1H), 8.05-7.68 (m, 3H), 7.56 (m, 4H), 6.21 (m, 1H), 4.70 (m, 2H), 4.40 (m, 2H), 3.97 (s, 1H), 3.56 (m, 2H), 2.91-2.85 (m, 5H), 2.02 (m, 2H), 1.59 (in, 2H).

### Synthesis of Compound 275

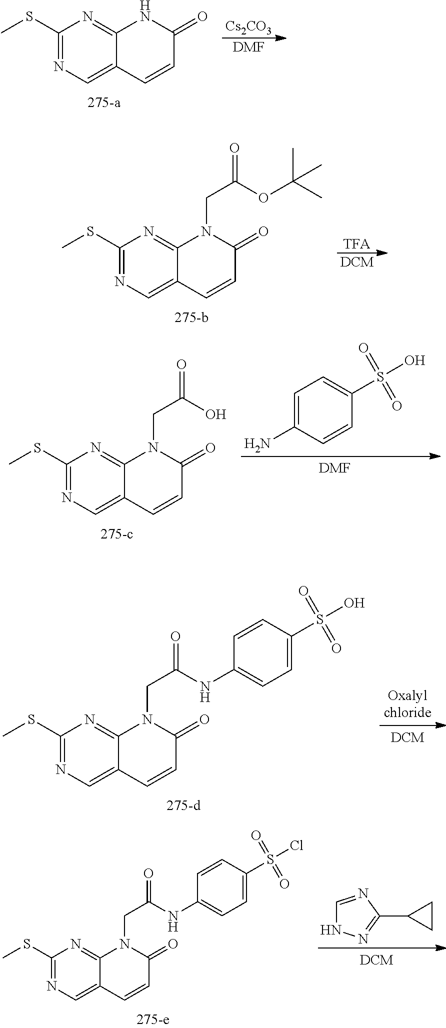

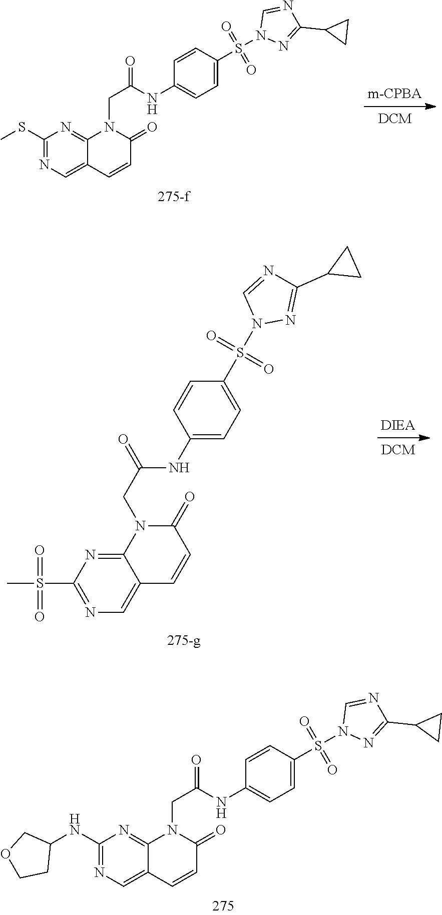 [SMILES: CSc1ncc2ccc(=O)n(CC(=O)Nc3ccc(S(=O)(=O)n4cnc(C5CC5)n4)cc3)c2n1 ; CS(=O)(=O)c1ncc2ccc(=O)n(CC(=O)Nc3ccc(S(=O)(=O)n4cnc(C5CC5)n4)cc3)c2n1 ; O=C(Cn1c(=O)ccc2cnc(NC3CCOC3)nc21)Nc1ccc(S(=O)(=O)n2cnc(C3CC3)n2)cc1]

### Step 1

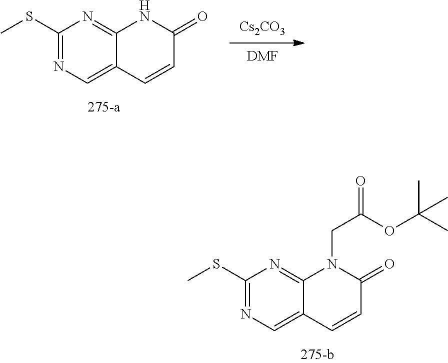

A mixture of 275-a (5.0 g, 25.88 mmol), tert-butyl 2-bromoacetate (5.7 mL, 38.81 mmol) and Cs₂CO₃ (16.86 g, 51.75 mmol) in DMF (30 mL) was stirred at 80° C. for 10 h. The mixture was partitioned between EtOAc (400 mL) and H₂O (300 mL). The organic layer was washed with H₂O (100 mL), dried (Na₂SO₄), filtered and evaporated to dryness. The residue was purified by silica gel flash chromatography (gradient 0%-50% EtOAc in hexanes) to afford 275-b (5.1 g, 64.12%) as a yellow oil. m/z: [M+H]⁺ Calcd for C₁₄H₁₇N₃O₃S 307.1; Found 308.2.

### Step 2

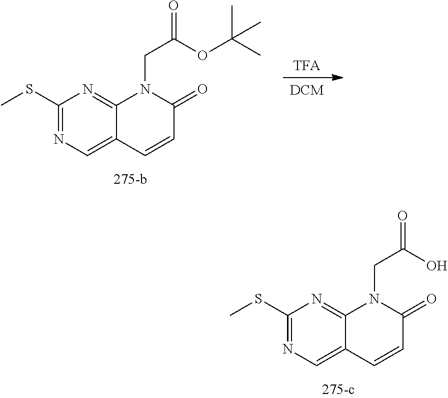

A mixture of 275-b (5.1 g, 16.59 mmol) in TFA (20 mL)/DCM (40 mL) was stirred at 20° C. for 10 h. After remove the solvent, 100 mL H₂O was added to the mixture. The residue was filtered to give 275-c (3.7 g, 88.75%) as a yellow solid. The crude product was used for the next step directly and without further purification. m/z: [M+H]⁺ Calcd for C₁₀H₉N₃O₃S 251.0; Found 252.1.

### Step 3

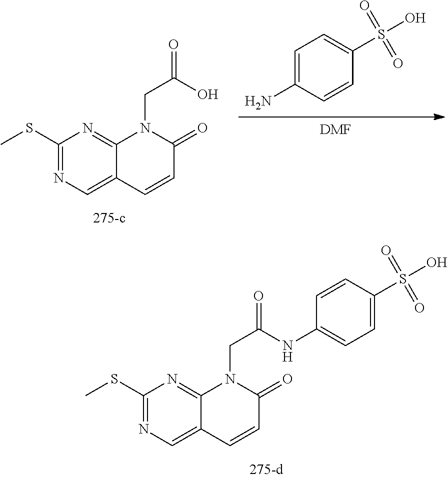

To a solution of 275-c (3.7 g, 14.72 mmol) and 4-aminobenzene-1-sulfonic acid (3.32 g, 19.14 mmol), DIPEA (12.2 mL, 73.63 mmol) in DCM (30 mL), DMF (30 mL) was added HATU (8.40 g, 22.09 mmol) under N2 with stirring. The mixture was stirred at 20° C. for 5 h. The reaction was purified by Prep-HPLC (C18, 0˜50% MeOH in H₂O with FA) to afford 275-d (3.5 g, 58.48%) as a yellow solid. m/z: [M+H]⁺ Calcd for C₁₆H₁₄N₄O₅S₂ 406.0; Found 407.1.

### Step 4

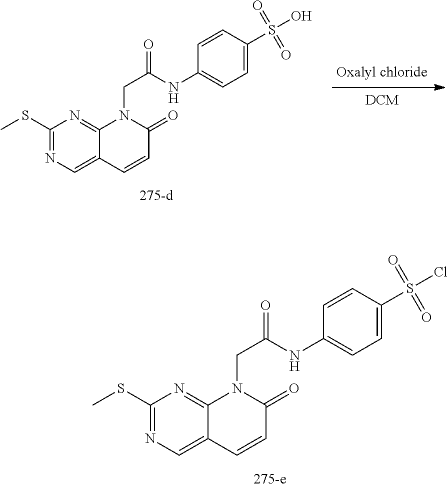

To a solution of 275-d (3.5 g, 8.61 mmol) in DMF (1 mL), DCM (30 mL) was added oxalyl chloride (13.0 mL, 25.83 mmol) under N2 with stirring. The mixture was stirred at 20° C. for 3 h. The suspension was filtered to give the filter cake 275-e (2.8 g, 76.53%) as a yellow solid. The crude product was used for the next step directly without further purification. m/z: [M+H]⁺ Calcd for C₁₆H₁₃ClN₄O₄S₂ 424.0; Found 425.1.

### Step 5

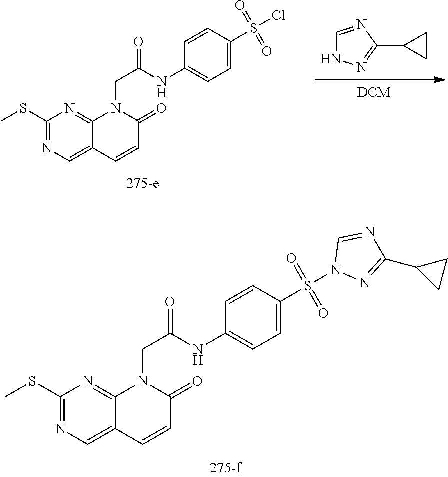

To a solution of 275-e (3.4 g, 8.37 mmol) and 3-cyclopropyl-1H-1,2,4-triazole (0.91 g, 8.36 mmol) in DCM (30 mL) was added DIPEA (4.2 mL, 25.09 mmol) under N₂ with stirring. The mixture was stirred at 20° C. for 8 h. The product was detected by LCMS. The residue was triturated with DCM and EtOAc, the suspension was filtered and give the filter cake 275-f (1.3 g, 31.23%) as a white solid. m/z: [M+H]⁺ Calcd for C₂₁H₁₉N₇O₄S₂ 497.1; Found 498.1. ¹H NMR (400 MHz, DMSO) δ 11.10 (s, 1H), 9.14 (s, 1H), 8.93 (s, 1H), 8.02 (dd, J=9.3, 2.2 Hz, 3H), 7.85 (d, J=9.0 Hz, 2H), 6.71 (d, J=9.5 Hz, 1H), 5.16 (s, 2H), 2.45 (s, 3H), 1.99 (td, J=8.4, 4.3 Hz, 1H), 0.95 (dd, J=8.3, 2.7 Hz, 2H), 0.84-0.77 (m, 2H).

### Step 6

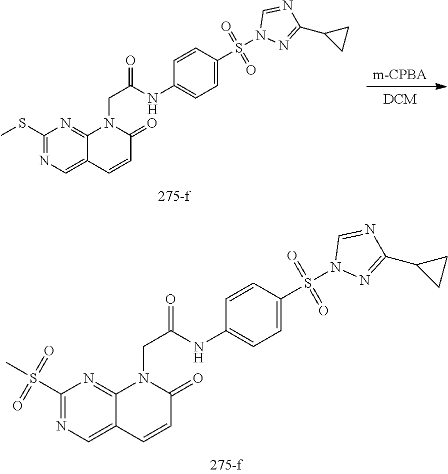

To a solution of 275-f (1.8 g, 3.62 mmol) in DCM (30 mL) was added m-CPBA (1.84 g, 9.04 mmol) under N₂ with stirring. The mixture was stirred at 20° C. for 10 h. The reaction was complete detected by LCMS. The residue was purified by silica gel flash chromatography (gradient 30%-100% EtOAc in DCM) to afford 275-g (1.2 g, 62.64%) as a white solid. m/z: [M+H]⁺ Calcd for C₂₁H₁₉N₇O₆S₂ 529.1; Found 530.1. ¹H NMR (400 MHz, DMSO) δ 11.11 (s, 1H), 9.37 (s, 1H), 9.14 (s, 1H), 8.22 (d, J=9.6 Hz, 1H), 8.02 (d, J=9.0 Hz, 2H), 7.84 (d, J=9.0 Hz, 2H), 7.01 (d, J=9.6 Hz, 1H), 5.21 (s, 2H), 3.40 (s, 3H), 2.02-1.99 (m, 1H), 0.98-0.91 (m, 2H), 0.83-0.76 (m, 2H).

### Step 7

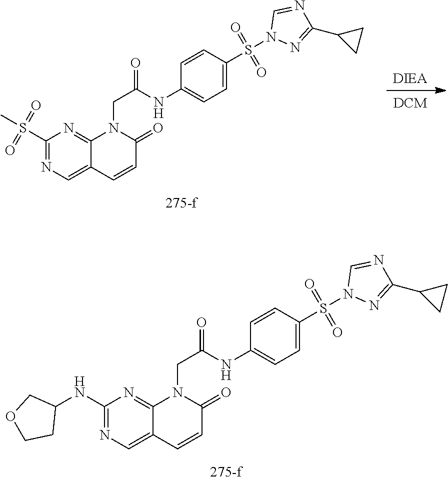

To a solution of 275-g (45 mg, 0.085 mmol) and oxolan-3-amine (8.2 mg, 0.093 mmol) in THF (5 mL) was added DIPEA (0.04 mL, 0.25 mmol) under N2 with stirring. The mixture was stirred at 20° C. for 1 h. The reaction mixture was purified by reverse-phase chromatography (SilaSep™ C18 silica flash cartridge, 40%-60% MeCN in H₂O with 0.1% formic acid) to afford 275 (25 mg, 54.83%) as a white solid. m/z: [M+H]⁺ Calcd for C₂₄H₂₄N₈O₅ 537.2; Found 538.1. ¹H NMR (400 MHz, DMSO) δ 11.06 (s, 1H), 9.13 (s, 1H), 8.67 (d, J=24.2 Hz, 1H), 8.15-7.98 (m, 3H), 7.88-7.80 (m, 3H), 6.34 (d, J=9.3 Hz, 1H), 5.18-4.96 (m, 2H), 4.36 (d, J=112.8 Hz, 1H), 3.73 (t, J=31.7 Hz, 2H), 3.46 (dd, J=27.1, 19.0 Hz, 2H), 1.99 (td, J=8.3, 4.3 Hz, 1H), 1.92-1.69 (m, 2H), 0.95 (dt, J=6.3, 4.0 Hz, 2H), 0.85-0.75 (m, 2H).

### Synthesis of Comp und 276

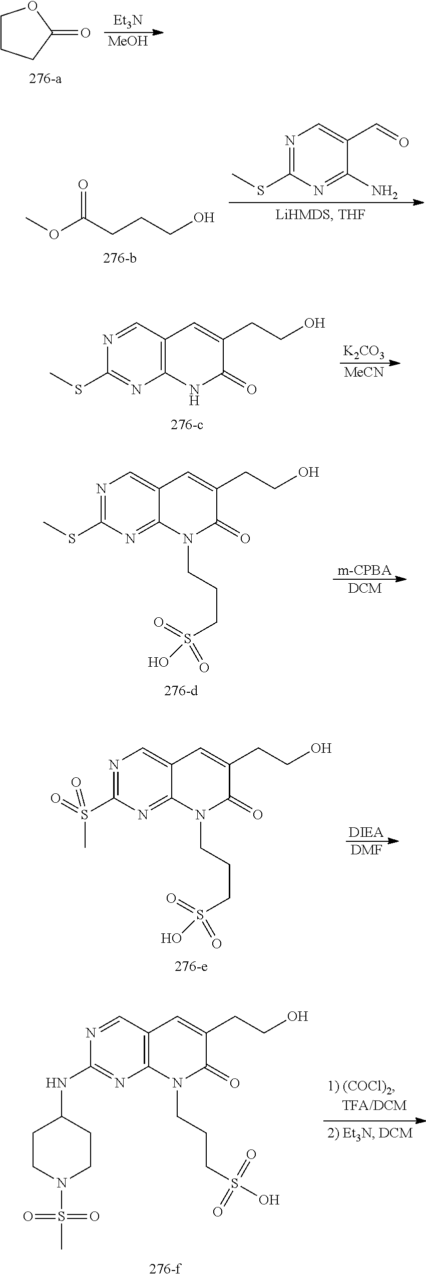

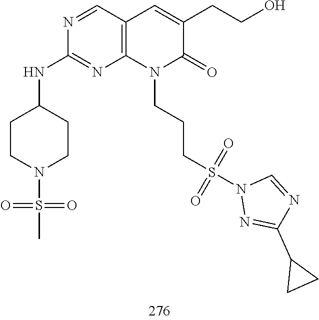 [SMILES: CS(=O)(=O)N1CCC(Nc2ncc3cc(CCO)c(=O)n(CCCS(=O)(=O)n4cnc(C5CC5)n4)c3n2)CC1]

### Step 1

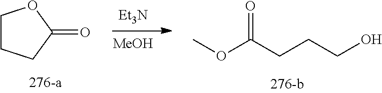

To a solution of 276-a (3 g, 34.85 mmol) in MeOH (10 mL) was added Et₃N (10 g, 99.01 mmol) with stirring at rt. After stirring at 60° C. for 16 h, the reaction mixture was concentrated and purified by silica gel flash chromatography (gradient 0%-30% EtOAc in hexanes) to afford 276-b (3 g, 72.88%) as a colorless oil. ¹H NMR (400 MHz, DMSO) δ 4.50 (t, J=5.2 Hz, 1H), 3.58 (s, 3H), 3.41-3.36 (m, 2H), 2.33 (t, J=7.5 Hz, 2H), 1.71-1.60 (m, 2H)

### Step 2

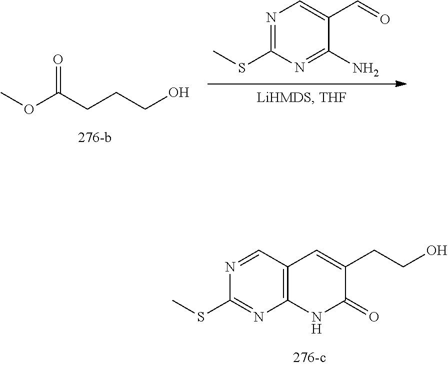

To a solution of 276-b (2.2 g, 18.62 mmol) in THF (20 mL) was added LiHMDS (1 M in THF) (7 mL) with stirring at −70° C. After stirring at rt for 10 min, 4-amino-2-(methylsulfanyl)pyrimidine-5-carbaldehyde (1.5 g, 8.86 mmol) was added and stirred at rt overnight. The reaction mixture was concentrated and purified by silica gel flash chromatography (gradient 0%-100% EtOAc in hexanes) to afford 276-c (1.2 g, 57.05%) as a yellow solid. m/z: [M+H]⁺ Calcd for C₁₀H₁₁N₃O₂S 237.1; Found 238.1.

### Step 3

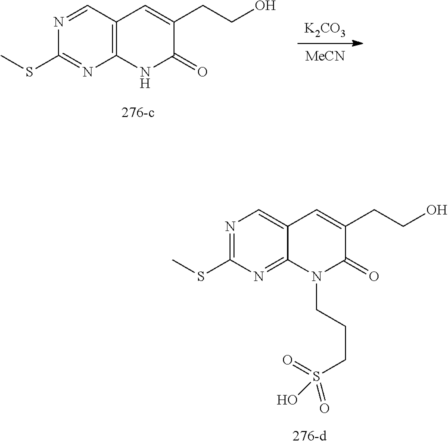

To a solution of 276-c (1.2 g, 5.06 mmol) and sodium 3-bromopropane-1-sulfonate (1.5 g, 6.66 mmol) in MeCN (20 mL) was added K2CO₃ (1.4 g, 10.13 mmol) with stirring at rt. After stirred at 80° C. for 2 h, the reaction mixture was concentrated and purified by reverse-phasechromatography (SilaSep™ C18 silica flash cartridge, 0%-30% MeOH in H₂O with 0.1% formic acid) to afford 276-d (1.3 g, 71.52%) as a yellow solid. m/z: [M+H]⁺ Calcd for C₁₃H₁₇N₃O₅S₂ 359.1; Found 360.1.

### Step 4

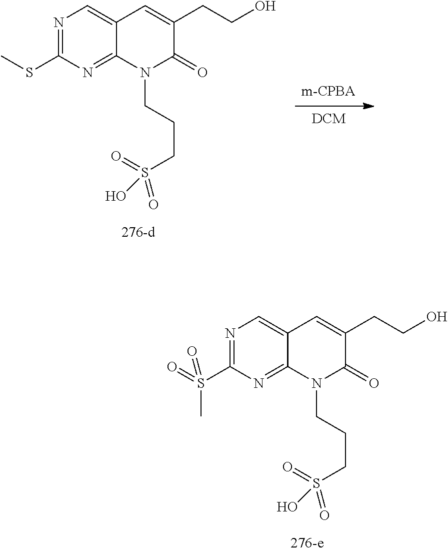

To a solution of 276-d (700 mg, 1.95 mmol) in DCM (50 mL) was added m-CPBA (1.1 g, 6.37 mmol) with stirring at rt. After stirring at rt for 1 h, the reaction mixture was concentrated and purified by reverse-phase chromatography (SilaSep™ C18 silica flash cartridge, 0%-10% MeOH in H₂O with 0.1% formic acid) to afford 276-e (700 mg, 91.82%) as a white solid. m/z: [M+H]⁺ Calcd for C₁₃H₁₇N₃O₇S₂ 391.1; Found 392.1.

### Step 5

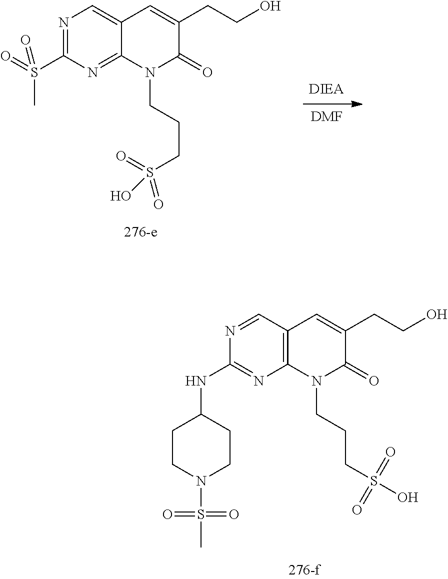

To a solution of 276-e (700 mg, 1.79 mmol) and 3-cyclopropyl-1H-1,2,4-triazole (23 mg, 0.21 mmol) in DMF (20 mL) was added K2CO₃ (50 mg, 0.36 mmol) and DIEA (500 mg, 3.87 mmol) with stirring at rt. After stirred at 80° C. for 16 h, the reaction mixture was concentrated and purified by reverse-phase chromatography (SilaSep™ C18 silica flash cartridge, 20%-40% MeCN in H₂O with 0.1% formic acid) to afford 276-f (500 mg, 57.11%) as a white solid. m/z: [M+H]⁺ Calcd for C₁₈H₂₇N₅O₇S₂ 489.1; Found 490.2.

### Step 6

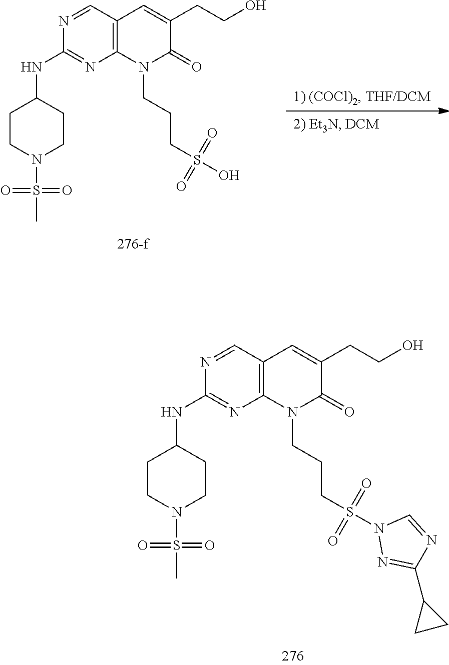

To a solution of 276-f (200 mg, 0.41 mmol) and 10 drops of DMF in DCM (10 mL) and THF (10 mL) was added oxalyl chloride (120 mg, 0.94 mmol) at 0° C., Then stirred at rt for 1 h. After stirring at rt, the mixture was concentrated. The mixture was added to 3-cyclopropyl-1H-1,2,4-triazole (60 mg, 0.55 mmol) and Et₃N (100 mg, 0.99 mmol) in DCM (10 mL). After stirring at rt for 1 h, the reaction mixture was concentrated and purified by reverse-phase chromatography (SilaSep™ C18 silica flash cartridge, 30%-50% MeCN in H₂O with 0.1% formic acid) to afford 276 (5 mg, 0.009 mmol, 2.11%) as a yellow solid. m/z: [M+H]⁺ Calcd for C₂₃H₃₂N₈O₆S₂ 580.2; Found 581.2. ¹H NMR (400 MHz, DMSO) δ 8.97 (s, 1H), 8.58 (s, 1H), 7.85-7.47 (m, 2H), 4.58 (t, J=5.4 Hz, 1H), 4.34 (s, 2H), 3.92 (s, 3H), 3.59 (m, 4H), 2.97-2.87 (m, 5H), 2.60 (t, J=6.6 Hz, 2H), 2.00 (m, 5H), 1.58 (m, 2H), 1.04-0.97 (m, 2H), 0.84 (m, 2H).

### Synthesis of Compound 285

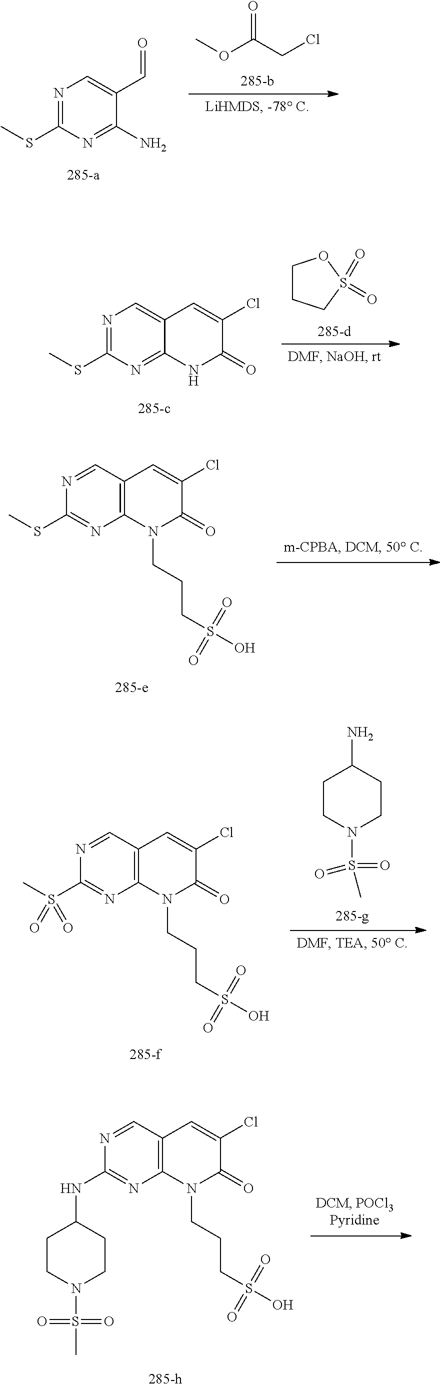

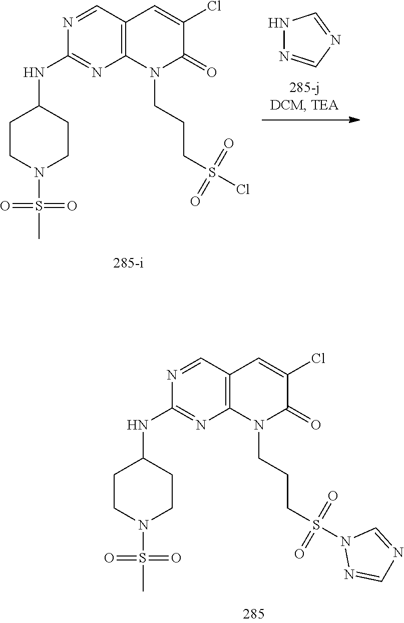

### Step 1

To a solution of 285-a (2.5 g, 14.8 mmol) in dried THF (30.0 mL), LiHMDS (29.6 ml, 29.6 mmol) added at −78° C. with N₂ protected. The resulting mixture stirred at −78° C. for 0.5 h. 285-b (1.76 g, 16.3 mmol) added. The resulting mixture stirred at −78° C. to rt for overnight. The reaction was quenched with ice water, then extracted with EA (2×200 mL) the organic layer was concentrated under reduced pressure to afford the crude product, which was purified by prep-TLC (PE/EA=1/1) to give 285-c (500.0 mg, 14.9% yield) as white solid. m/z: [M+H]⁺ Calcd for C₈H₆ClN₃OS 226.9; Found 227.9.

### Step 2

To a solution of 285-c (500.0 mg, 2.20 mmol) in DMF (10.0 mL), 285-d (296.0 mg, 2.42 mmol), NaOH (440.0 mg, 11.0 mmol) was added at 0° C. The resulting mixture stirred at rt for 3 h. The reaction was quenched with brine, extracted with EA (2×200 mL) the organic layer was concentrated under reduced pressure to afford the crude product, which was purified by flash chromatography on silica gel (DCM/MeOH=1/0 to 4/1) to give 285-e (500.0 mg, 65.2% yield) as oiled. m/z: [M+H]⁺ Calcd for C₁₁H₁₂ClN₃O₄S₂ 349.0; Found 348.0.

### Step 3

To a solution of 285-e (500.0 mg, 1.43 mmol) in DCM (10.0 mL), m-CPBA (494.5 mg, 2.87 mmol) added at 0° C. The resulting mixture stirred at 50° C. overnight. extracted with DCM (2×100 mL) the organic layer was concentrated under reduced pressure to afford the crude product, which was purified by flash chromatography on silica gel (DCM/MeOH=1/0 to 4/1) to give 285-f (490.0 mg, 90.0% yield). m/z: [M+H]⁺ Calcd for C₁₁H₁₂ClN₃O₆S₂ 380.9; Found 381.9.

### Step 4

To a solution of 285-f (490.0 mg, 1.29 mmol) in DMF (10.0 mL), 285-g (229.6 mg, 1.29 mmol), TEA (260.5 mg, 2.58 mmol) was added at 0° C. The resulting mixture stirred at 50° C. for 2 h, extracted with EA (2×100 mL) the organic layer was concentrated under reduced pressure to afford the crude product, which was purified by flash chromatography on silica gel (DCM/MeOH=1/0 to 4/1) to give 285-h (450.0 mg, 72.8% yield). m/z: [M+H]⁺ Calcd for C₁₆H₂₂ClN₅SO₆S₂ 479.07; Found 480.07.

### Step 5

To a solution of 285-h (120.0 mg, 0.25 mmol) in POCl₃ (0.5 mL), pyridine (0.2 mL) added at 0° C. The resulting mixture stirred at rt for 1 h, concentrated under reduced pressure to afford the crude product, extracted with DCM (2×50 mL) the organic layer was concentrated under reduced pressure to afford 285-i (80.0 mg, 64.5% yield). m/z: [M+H]⁺ Calcd for C₁₆H₂₁Cl₂N₅O₅S₂ 497.04; Found 498.04.

### Step 6

To a solution of 285-i (80.0 mg, 0.16 mmol) in DCM (3.0 mL), 285-j (11.0 mg, 0.16 mol), TEA (32.3 mg, 0.32 mmol) was added. The resulting mixture stirred at rt for 1 h, then extracted with DCM (2×50 mL) the organic layer was concentrated under reduced pressure to afford the crude product, which was purified by Prep-TLC (DCM/MeOH=10/1) to give 285 (6.0 mg, 7.1% yield) as white solid. m/z: [M+H]⁺ Calcd for C₁₈H₂₃ClN₈O₅S₂ 530.09; Found 531.09. ¹H NMR (400 MHz, DMSO) δ 9.20 (s, 1H), 8.61 (s, 1H), 8.39 (d, J=11.6 Hz, 1H), 8.09 (d, J=8.5 Hz, 2H), 4.32 (d, J=29.8 Hz, 2H), 4.00 (d, J=7.4 Hz, 3H), 3.57 (d, J=11.6 Hz, 2H), 2.88 (d, J=6.9 Hz, 4H), 1.98 (dd, J=26.9, 8.5 Hz, 5H), 1.58 (d, J=10.3 Hz, 2H).

### Synthesis of Compound 288

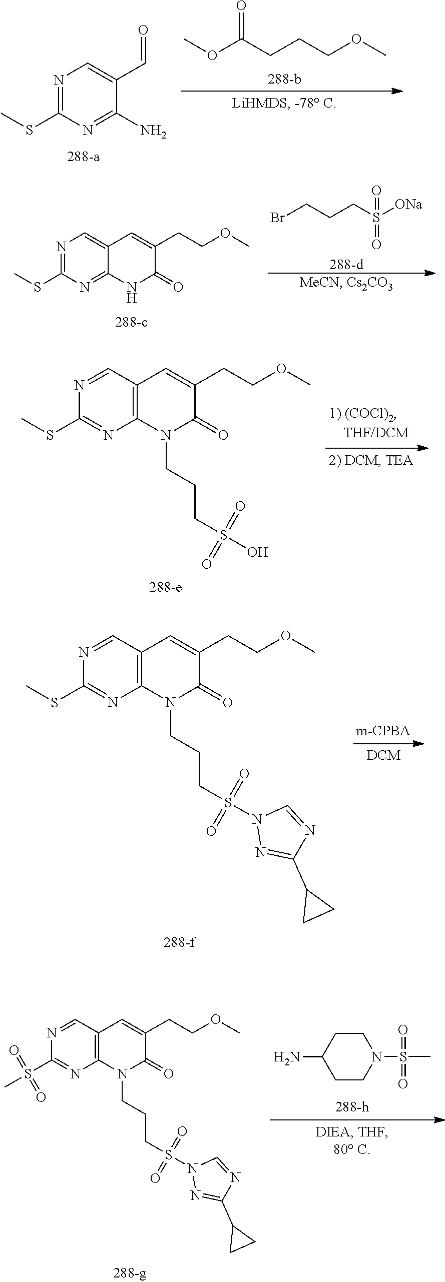

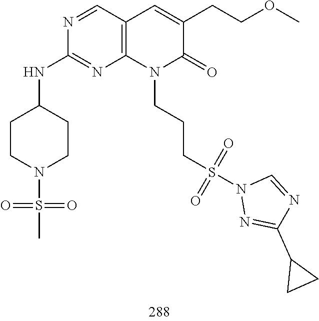 [SMILES: COCCc1cc2cnc(NC3CCN(S(C)(=O)=O)CC3)nc2n(CCCS(=O)(=O)n2cnc(C3CC3)n2)c1=O]

### Step 1

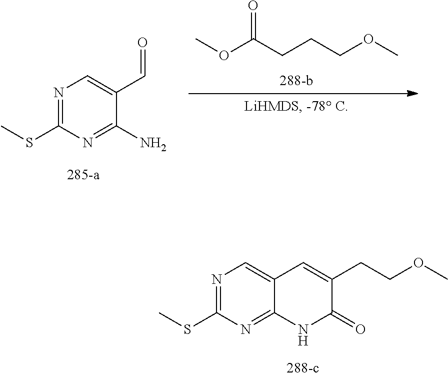

To a solution of 288-a (1.8 mL, 14.2 mmol) in dry THF (50 mL) was added 288-b (3.8 g, 28.4 mmol) and LiHMDS (12 mL, 56.7 mmol). The reaction was stirred at −78° C. for overnight. Ice-water was added thereto, the mixture was extracted with ethyl acetate (30 mL×2). The organic layer was washed with brine (30 ml), dried over anhydrous Na₂SO₄, filtered and concentrated in vacuo to afford 288-c (1.8 mL, 14.2 mmol). The water was adjusted PH=6 with 1 mol/L diluted hydrochloric acid, and then the mixture was extracted with ethyl acetate (30 ml×2). The organic layer was washed with brine (30 ml), dried over anhydrous Na₂SO₄, filtered and concentrated in vacuo. The residue was purified by silica gel flash chromatography (gradient 0%-50% EtOAc in DCM) to afford 288-c (700 mg, 19.7%) as a white solid. m/z: [M+H]⁺ Calcd for C₁₁H₁₃N₃O₂S 251.1; Found 251.1.

### Step 2

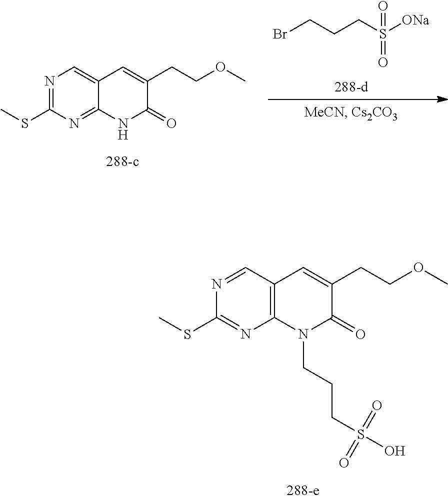

To a solution of 288-c (700 mg, 2.8 mmol) in MeCN (15 mL) was added 288-d (752.2 mg, 3.3 mmol) and Cs₂CO₃ (1.8 g, 5.56 mmol). The reaction was stirred at 80° C. overnight. Then the suspension is filtered and the filtrate is concentrated in vacuo.

The residue was purified by reverse phase chromatography (SilaSep™ C18 silica flash cartridge, 40%-60% MeCN in H₂O) to afford 288-e (800 mg, 76.9%) as a yellow oil. m/z: [M+H]⁺ Calcd for C₁₄H₁₉N₃O₅S₂ 373.1; Found 374.1.

### Step 3

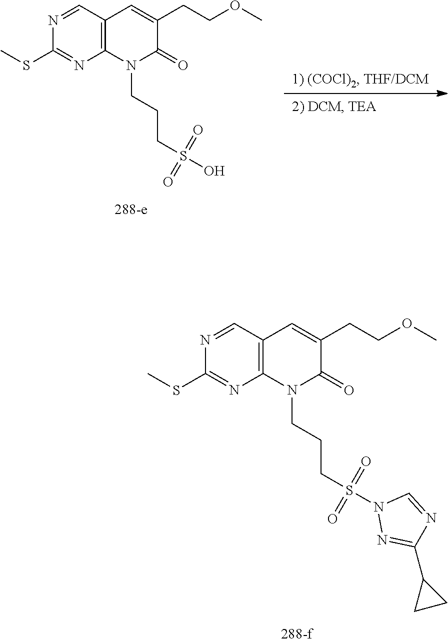

To a solution of 288-e (190 mg, 0.5 mmol) in dry THF (3 mL) and DCM (3 mL) was added (COCl)₂ (0.7 mL). The reaction was stirred at rt for 1 h. And then the residue was concentrated in vacuo and TEA (0.2 mL, 1.5 mmol) in dry DCM (5 mL) was added. The reaction was stirred at rt for 1 h. Then the residue was concentrated in vacuo. The residue was purified by silica gel flash chromatography (gradient 0%-50% EtOAc in hexanes) to afford 288-f (150 mg, 43.6%) as a colourless oil. m/z: [M+H]⁺ Calcd for C₁₉H₂₄N₆O₄S₂ 464.1; Found 465.2.

### Step 4

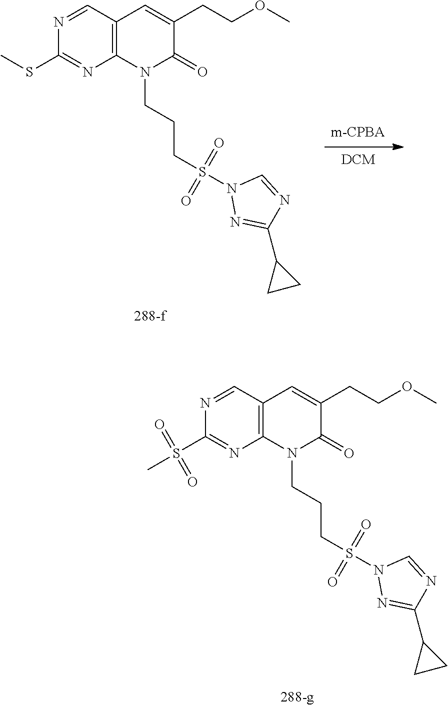

To a solution of 288-f (150 mg, 0.3 mmol) in DCM (7 mL) was added m-CPBA (0.2 mL, 0.8 mmol). The reaction was stirred at rt for 1 h. And then the residue was concentrated in vacuo. The residue was purified by silica gel flash chromatography (gradient 0%-20% EtOAc in DCM) to afford 288-g (109 mg, 68%) as a colourless oil. m/z: [M+H]⁺ Calcd for C₁₉H₂₄N₆O₆S₂ 496.1; Found 497.1.

### Step 5

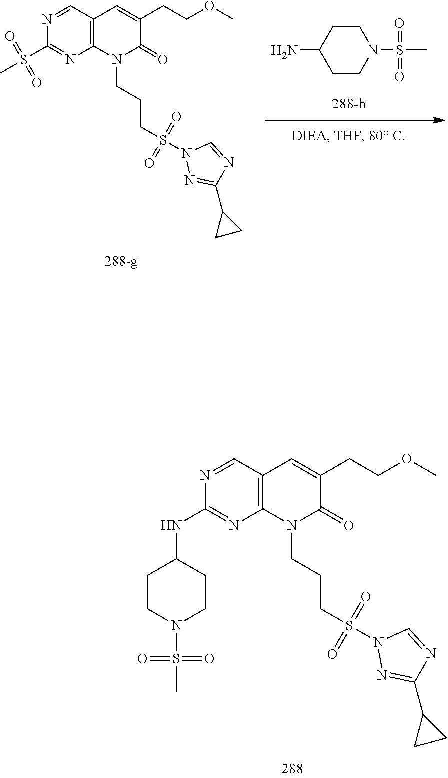 [SMILES: COCCc1cc2cnc(S(C)(=O)=O)nc2n(CCCS(=O)(=O)n2cnc(C3CC3)n2)c1=O ; CS(=O)(=O)N1CCC(N)CC1 ; COCCc1cc2cnc(NC3CCN(S(C)(=O)=O)CC3)nc2n(CCCS(=O)(=O)n2cnc(C3CC3)n2)c1=O]

To a solution of 288-g (90 mg, 0.2 mmol) in dry THF (5 mL) was added 288-h (36 mg, 0.2 mmol) and DIEA (0.1 mL, 0.2 mmol). The reaction was stirred at rt for 1 h. And then the residue was concentrated in vacuo. The residue was purified by reverse-phase chromatography (SilaSep™ C18 silica flash cartridge, 30%-60% MeCN in H₂O) to afford 288 (4.6 mg, 4.3%). m/z: [M+H]⁺ Calcd for C₂₄H₃₄N₈O₆S₂ 594.2; Found 595.3. 1H NMR (400 MHz, DMSO) δ 8.96 (s, 1H), 8.57 (s, 1H), 7.83 (d, J=7.4 Hz, 1H), 7.61 (s, 1H), 4.33 (s, 2H), 3.92 (s, 3H), 3.58-3.47 (m, 4H), 3.24 (s, 3H), 2.91 (d, J=17.3 Hz, 5H), 2.68 (t, J=6.4 Hz, 2H), 2.04-1.91 (m, 5H), 1.57 (d, J=10.9 Hz, 2H), 1.02-0.96 (m, 2H), 0.83 (s, 2H).

### Synthesis of Compound 290

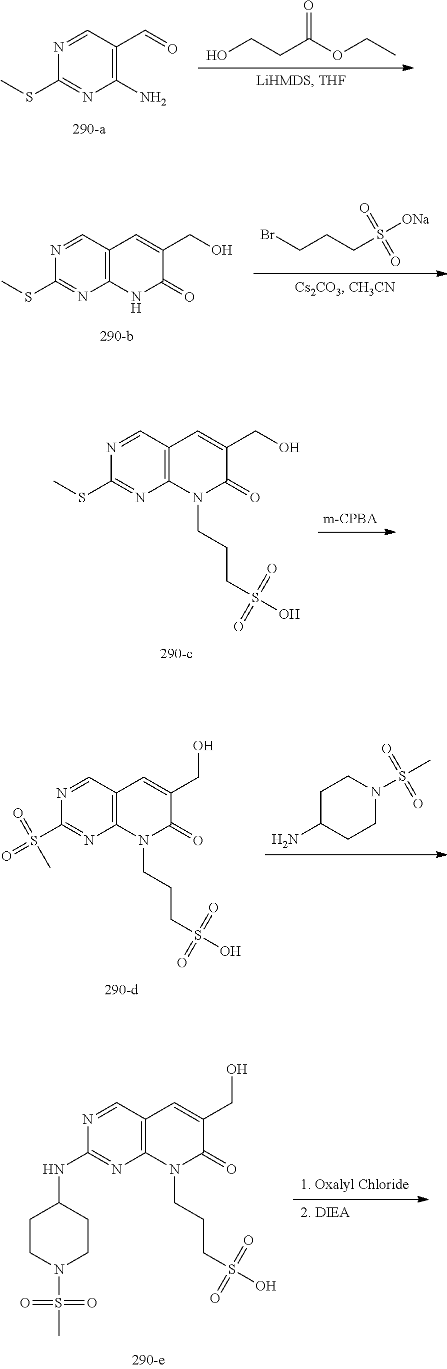

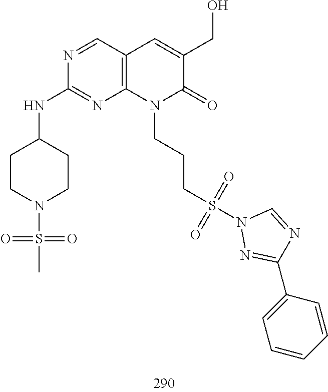 [SMILES: CS(=O)(=O)N1CCC(Nc2ncc3cc(CO)c(=O)n(CCCS(=O)(=O)n4cnc(-c5ccccc5)n4)c3n2)CC1]

### Step 1

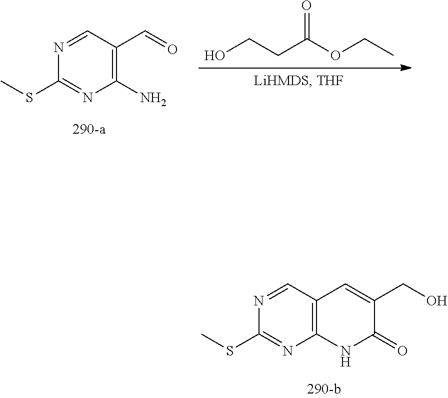

To a solution of ethyl 3-hydroxypropanoate (0.6 mL, 4.88 mmol) in THF (10 mL) was added Lithium bis(trimethylsilyl)amide, 1M solution in THF (6.6 mL) under N₂ with stirring at −78′° C. After stirring at −78° C. for 0.5 h, 290-a (750 mg, 4.43 mmol) was added to the reaction. The mixture was stirred at 20° C. for another 10 h. The reaction was complete detected by LCMS. The reaction mixture was quenched by saturated NH₄Cl solution (50 mL). The residue was partitioned between EtOAc (30 mL×3) and H₂O (30 mL). The organic layer was washed with H₂O (30 mL), dried over (MgSO₄), filtered and evaporated to dryness. The residue was purified by silica gel flash chromatography (gradient 0%-100% EtOAc in hexanes) to afford 290-b (320 mg, 1.433 mmol, 32.34%) as a yellow solid. m/z: [M+H]⁺ Calcd for C₉H₉N₃O₂S 223.0; Found 224.1.

### Step 2

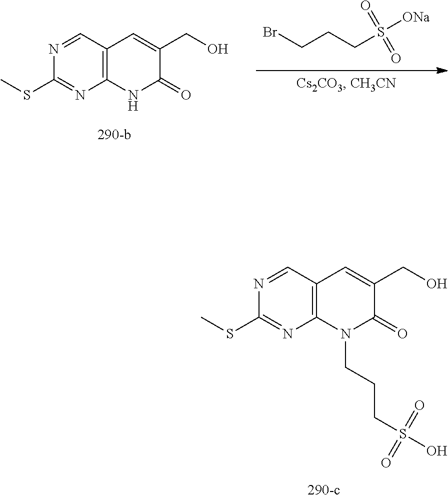

A mixture of 290-b (0.8 g, 3.58 mmol), sodium 3-bromopropane-1-sulfonate (1.21 g, 5.38 mmol) and Cs₂CO₃ (2.33 g, 7.17 mmol) in CH₃CN (20 mL) was stirred at 80° C. for 10 h. The reaction was purified by reverse-phase chromatography (SilaSep™ C18 silica flash cartridge, 0˜50% MeOH in H₂O with FA) to afford 290-c (1.05 g, 84.84%) as a yellow solid. m/z: [M+H]⁺ Calcd for C₁₂H₁₅N₃O₅S₂ 345.0; Found 346.1.

### Step 3

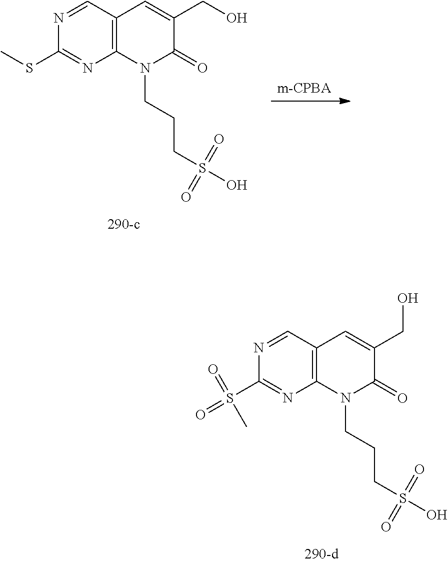 [SMILES: CSc1ncc2cc(CO)c(=O)n(CCCS(=O)(=O)O)c2n1 ; "m-CPBA" ; CS(=O)(=O)c1ncc2cc(CO)c(=O)n(CCCS(=O)(=O)O)c2n1]

To a solution of 290-c (1.3 g, 3.76 mmol) in DCM (30 mL) was added m-CPBA (1.53 g, 7.53 mmol) under N₂ with stirring. The mixture was stirred at 20° C. for 10 h. The reaction was purified by reverse-phase chromatography (SilaSep™ C18 silica flash cartridge, 0˜50% MeOH in H₂O with FA) to afford 290-d (1.05 g, 73.92%) as a yellow solid. m/z: [M+H]⁺ Calcd for C₁₂H₁₅N₃O₇S₂ 377.0; Found 378.1.

### Step 4

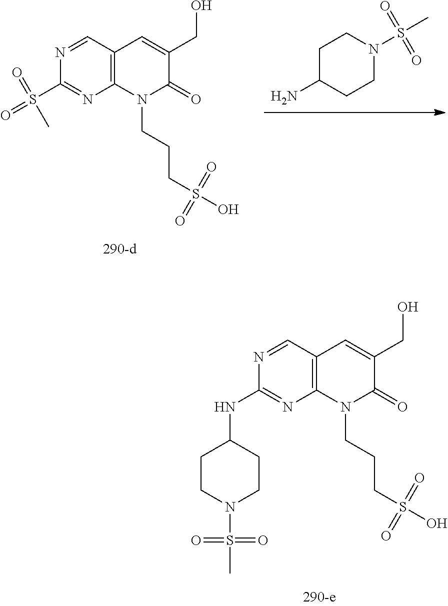 [SMILES: CS(=O)(=O)c1ncc2cc(CO)c(=O)n(CCCS(=O)(=O)O)c2n1 ; CS(=O)(=O)N1CCC(N)CC1 ; CS(=O)(=O)N1CCC(Nc2ncc3cc(CO)c(=O)n(CCCS(=O)(=O)O)c3n2)CC1]

A mixture of 290-d (1.4 g, 3.71 mmol), 1-methanesulfonylpiperidin-4-amine (0.99 g, 5.56 mmol) and THF (15 mL), DMF (15 mL) was stirred at 80° C. for 10 h. The reaction was purified by reverse-phase chromatography (SilaSep™ C18 silica flash cartridge, 0˜50% MeOH in H₂O with FA) to afford 290-e (720 mg, 40.81%) as a yellow solid. m/z: [M+H]⁺ Calcd for C₁₇H₂₅N₅O₇S₂ 475.1; Found 476.1. ¹H NMR (400 MHz, DMSO) δ 8.65 (s, 1H), 7.81-7.63 (m, 2H), 5.20 (s, 1H), 4.35 (s, 2H), 4.29 (s, 2H), 4.01 (d, J=7.1 Hz, 1H), 3.54-3.38 (m, 2H), 3.05-2.98 (m, 2H), 2.89 (d, J=5.4 Hz, 5H), 1.98-1.72 (m, 4H), 1.55-1.41 (in, 2H).

### Step 5

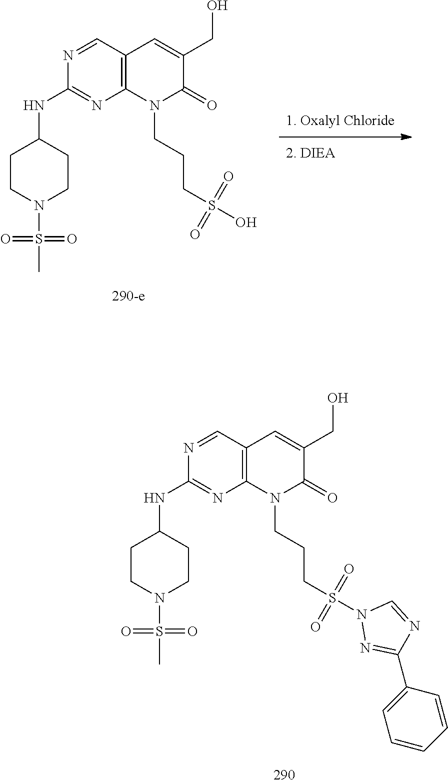 [SMILES: CS(=O)(=O)N1CCC(Nc2ncc3cc(CO)c(=O)n(CCCS(=O)(=O)O)c3n2)CC1 ; CS(=O)(=O)N1CCC(Nc2ncc3cc(CO)c(=O)n(CCCS(=O)(=O)n4cnc(-c5ccccc5)n4)c3n2)CC1]

To a solution of 290-e (120 mg, 0.25 mmol) in DCM (5 mL), DMF (0.3 mL) was added oxalyl chloride (0.38 mL, 0.76 mmol) under N₂ with stirring. The mixture was stirred at 20° C. for 1 h. After removal of the solvent, 3-phenyl-1H-1,2,4-triazole (55 mg, 0.38 mmol) DCM (5 mL) was added to the mixture, then DIPEA (0.2 mL, 1.26 mmol) was added and stirred at 20° C. for 3 h.

The reaction mixture was purified by reverse-phase chromatography (SilaSep™ C18 silica flash cartridge, 40%-60% MeCN in H₂O with 0.1% formic acid) to afford 290 (5.8 mg, 3.66%) as a white solid. m/z: [M+H]⁺ Calcd for C₂₅H₃₀N₈O₆S₂ 602.2; Found 603.1. ¹H NMR (400 MHz, DMSO) δ 9.24 (s, 1H), 8.58 (s, 1H), 8.04-7.98 (m, 2H), 7.81 (d, J=7.6 Hz, 1H), 7.65 (s, 1H), 7.54 (d, J=4.9 Hz, 3H), 5.18 (t, J=5.3 Hz, 1H), 4.42-4.23 (m, 4H), 3.99-3.78 (m, 3H), 3.55-3.35 (m, 2H), 2.95-2.86 (m, 5H), 2.09 (d, J=5.6 Hz, 2H), 1.93-1.71 (m, 2H), 1.54-1.32 (in, 2H).

### Synthesis of Compound 291

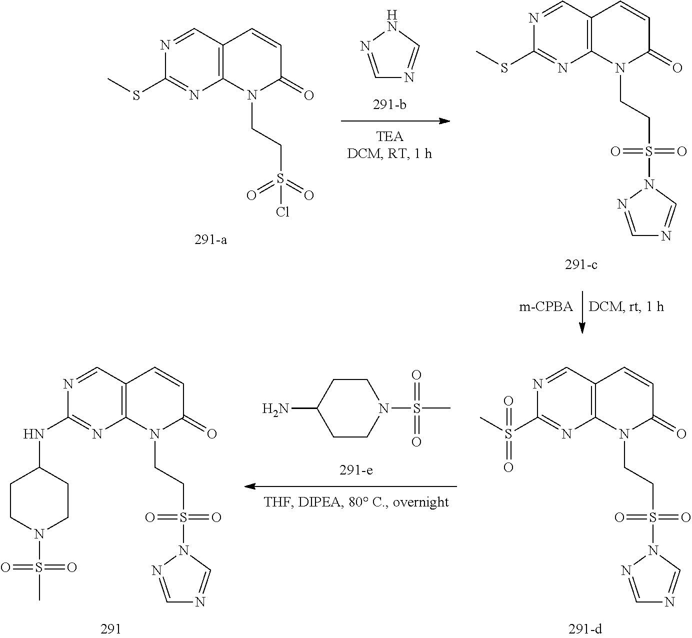

### Step 1

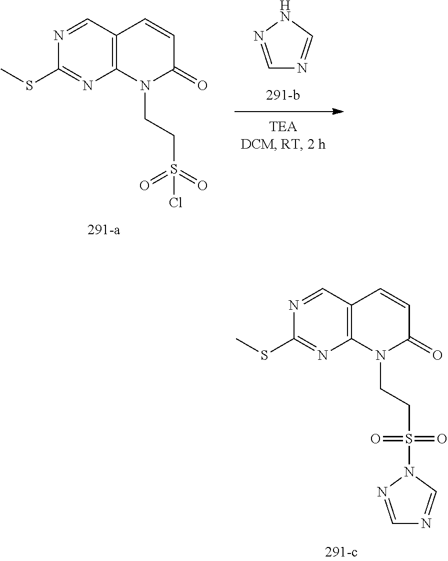 [SMILES: CSc1ncc2ccc(=O)n(CCS(=O)(=O)Cl)c2n1 ; c1nc[nH]n1 ; CSc1ncc2ccc(=O)n(CCS(=O)(=O)n3cncn3)c2n1]

To a stirred solution of 291-a (50 mg, 0.16 mmol) and 291-b (0.01 mL, 0.16 mmol) in DCM (7 mL) was added TEA (0.05 mL, 0.31 mmol) under N2. After stirring at rt for 2 h. The residue was purified by silica gel flash chromatography (gradient 0%-10% MeOH in DCM) to afford 291-c (45 mg, 81.67%) as a yellow solid. LC/MS (ESI) m/z: 353 (M+H)⁺.

### Step 2

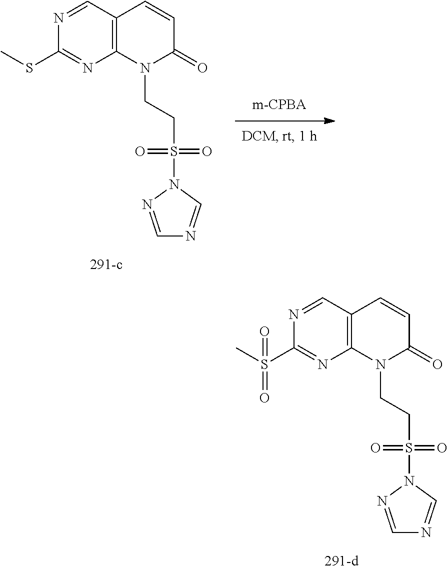 [SMILES: CSc1ncc2ccc(=O)n(CCS(=O)(=O)n3cncn3)c2n1 ; CS(=O)(=O)c1ncc2ccc(=O)n(CCS(=O)(=O)n3cncn3)c2n1]

To a stirred solution of 291-c (43 mg, 0.12 mmol) and m-CPBA (0.19 mL, 0.610 mmol) in DCM (5 mL). After stirring at rt for 1 h. The reaction mixture was purified by silica gel flash chromatography (gradient 0%-10% MeOH in DCM) to afford 291-d (40 mg, 85.28%) as a yellow solid. LC/MS (ESI) m/z: 385 (M+H)⁺.

### Step 3

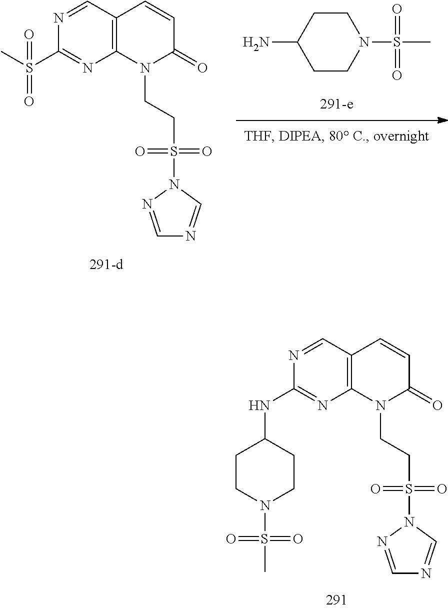 [SMILES: CS(=O)(=O)c1ncc2ccc(=O)n(CCS(=O)(=O)n3cncn3)c2n1 ; CS(=O)(=O)N1CCC(N)CC1 ; CS(=O)(=O)N1CCC(Nc2ncc3ccc(=O)n(CCS(=O)(=O)n4cncn4)c3n2)CC1]

To a stirred solution of 291-d (40 mg, 0.10 mmol) and 291-e (18.6 mg, 0.10 mmol) in THF (6 mL) was added DIEA (0.02 mL, 0.10 mmol) under N₂. After stirring at 80° C. overnight. The residue was purified by reverse-phase chromatography (SilaSep™ C18 silica flash cartridge, 40%-60% MeCN in H₂O with 0.1% formic acid)) to afford 291 (2.4 mg, 4.78%) as a white solid. LC/MS (ESI) ml/z: 483 (M+H)+. ¹H NMR (400 MHz, DMSO) δ 9.17 (s, 1H), 8.60 (d, J=13.8 Hz, 1H), 8.39 (d, J=29.3 Hz, 1H), 8.04 (d, J=7.8 Hz, 1H), 7.74 (d, J=9.4 Hz, 1H), 6.24 (d, J=9.4 Hz, 1H), 4.64 (m, 2H), 4.29 (m, 2H), 3.96 (m, 1H), 3.59 (m, 2H), 2.95-2.85 (in, 5H), 2.03 (m, 2H), 1.67-1.53 (in, 2H).

### Synthesis of Corn und 298

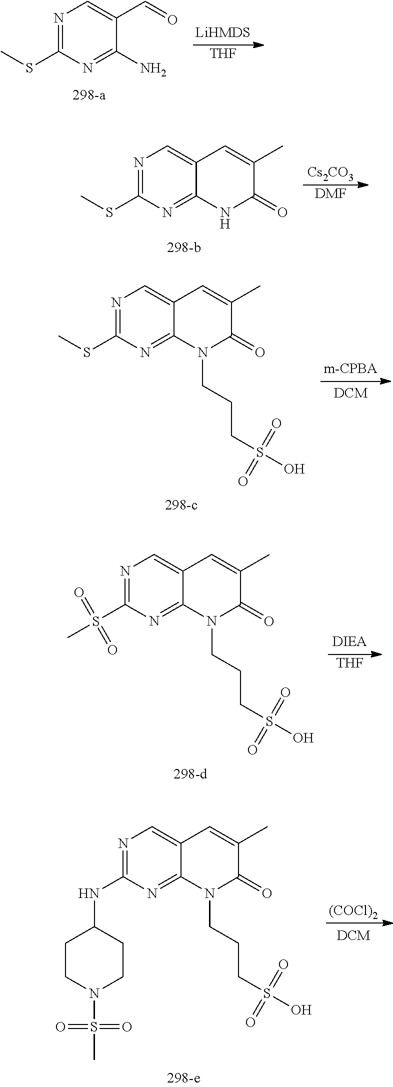

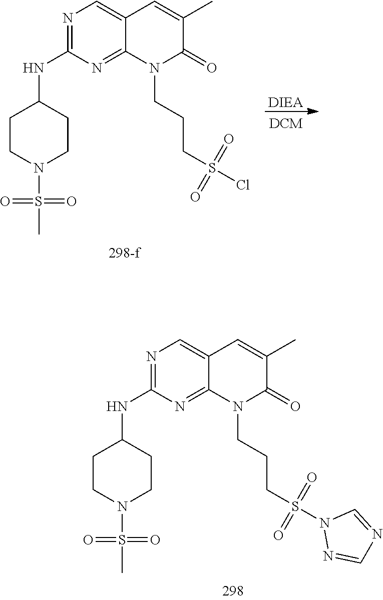 [SMILES: Cc1cc2cnc(NC3CCN(S(C)(=O)=O)CC3)nc2n(CCCS(=O)(=O)Cl)c1=O ; "DIEA" ; Cc1cc2cnc(NC3CCN(S(C)(=O)=O)CC3)nc2n(CCCS(=O)(=O)n2cncn2)c1=O]

### Step 1

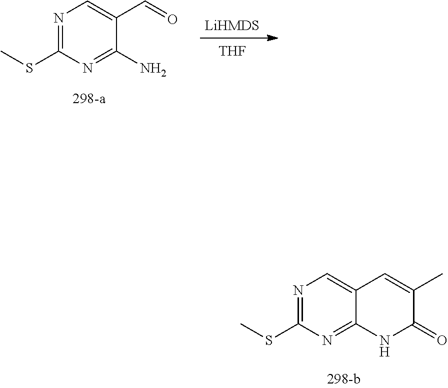

To a solution of methyl propionate (1.87 g, 21.25 mmol) in THF (20 mL) at −78° C., LiHDMS (26.6 mL, 1M) was added dropwise. the mixture was stirred at −78° C. for 30 min, 298-a (3 g, 17.73 mmol) was added. the reaction stirred at 20° C. overnight. The reaction mixture was quenched by saturated NH₄Cl solution, the residue was partitioned between EtOAc (30 mL×3) and NH₄Cl solution (50 mL). The organic layer was washed with H₂O, dried with Na₂SO₄. The residue was purified by column chromatography on silica gel eluted with PE/EtOAc (1:0-1:1) to afford 298-b (572 mg, 15.57%). as a white solid. m/z: [M+H]⁺ Calcd for C₉H₉N₃OS 207.0; Found 208.0.

### Step 2

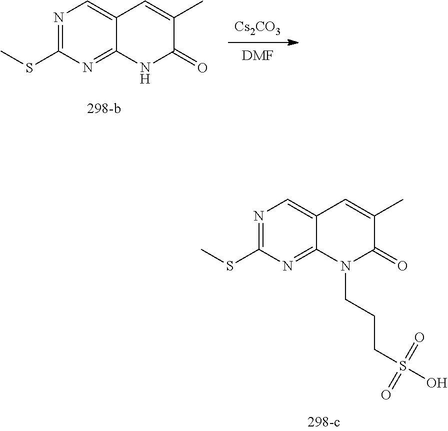

To a solution of 298-b (572 mg, 2.76 mmol) and sodium 3-bromopropanesulfonate (931.7 mg, 4.14 mmol) in DMF (10 mL). Cs₂CO₃ (1.8 g, 5.53 mmol) was added. The mixture was stirred at 80° C. overnight. The reaction was purified by reverse-phase chromatography (SilaSep™ C18 silica flash cartridge, 0-50% MeOH in H₂O with FA) to afford 298-c (564 mg, 62.04%) as a white solid. m/z: [M+H]⁺ Calcd for C₁₂H₁₅N₃O₄S₂ 329.0; Found 330.0.

### Step 3

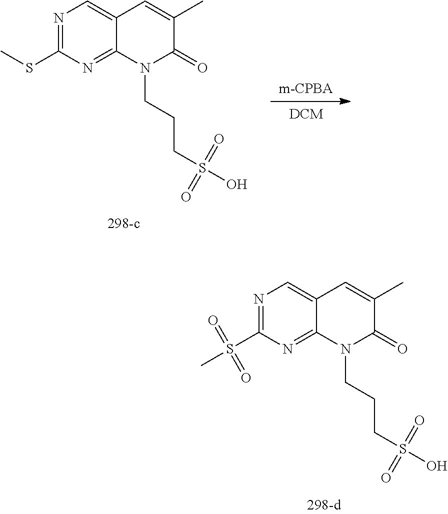

To a solution of 298-c (564 mg, 1.71 mmol) in DCM (10 mL) was added m-CPBA (698 mg, 4.04 mmol) with stirring at rt. the mixture was stirred at 20° C. overnight, the reaction was purified by reverse-phase chromatography (SilaSep™ C18 silica flash cartridge, 0-50% MeOH in H₂O with FA) to afford 298-d (520 mg, 87.03%) as a yellow solid. m/z: [M+H]⁺ Calcd for C₁₇H₁₄N₆O₅S₂ 361.0; Found 362.0.

### Step 4

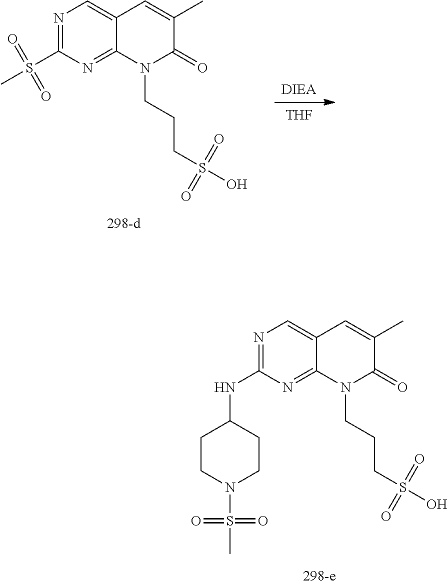 [SMILES: Cc1cc2cnc(S(C)(=O)=O)nc2n(CCCS(=O)(=O)O)c1=O ; "DIEA" ; Cc1cc2cnc(NC3CCN(S(C)(=O)=O)CC3)nc2n(CCCS(=O)(=O)O)c1=O ; "THF"]

To a solution of 298-d (520 mg, 1.44 mmol) and DIPEA (0.5 mL, 2.88 mmol) in THF (20 mL) was added 1-methanesulfonylpiperidin-4-amine (282 mg, 1.58 mmol) with stirring at rt. After stirred at 80° C. for 1 h, the reaction mixture was concentrated and purified by reverse-phase chromatography (SilaSep™ C18 silica flash cartridge, 40%-60% MeCN in H₂O with 0.1% formic acid) to afford 298-e (257 mg, 38.94%) as a white solid. m/z: [M+H]⁺ Calcd for C₁₇H₂₅N5O₆S₂ 459.0; Found 460.0.

### Step 5

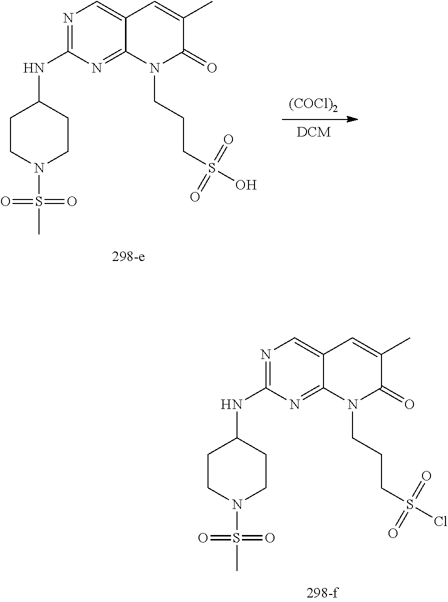

To a solution of 298-e (150 mg, 0.327 mmol) in DMF (0.5 mL), oxalyl chloride (0.8 mL, 1.59 mmol) in DCM (10 mL) was added under N₂ with stirring. The mixture was stirred at 20° C. for 3 h. The suspension was filtered to give the filter cake 298-f (146 mg, 93.60%) as a yellow solid. The crude product was used for the next step directly without further purification. m/z: [M+H]⁺ Calcd for C₁₇H₂₄ClN₅O₅S₂ 477.0; Found 478.1.

### Step 6

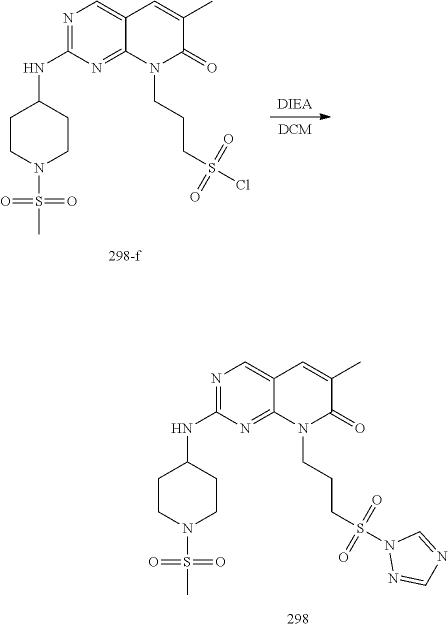

To a solution of 298-f (146 mg, 0.31 mmol) and 1H-1,2,4-triazole (27.4 mg, 0.40 mmol) in DCM (5 mL) was added DIPEA (0.16 mL, 1.00 mmol) under N₂ with stirring. The mixture was stirred at 20° C. for 3 h. The reaction was purified by reverse-phase chromatography (SilaSep™ C18 silica flash cartridge, 0-50% acetonitrile in H₂O with FA) to afford 298 (35.6 mg, 22.82%) as a white solid. m/z: [M+H]⁺ Calcd for C₁₉H₂₆N₈O₅S₂ 510.0; Found 511.1. ¹H NMR (400 MHz, DMSO) δ 9.20 (s, 1H), 8.53 (s, 1H), 9.14 (s, 1H), 8.39 (s, 1H), 7.79 (d, J=7.3 Hz, 1H), 7.59 (s, 1H), 4.32 (s, 2H), 4.32 (s, 2H), 3.99 (s, 3H), 3.56 (d, J=12.3 Hz, 2H), 2.95-2.87 (m, 5H), 2.03 (s, 7H), 1.57 (d, J=9.6 Hz, 2H).

TABLE 1
Non-limiting heteroaryl sulfonyl compounds of the present invention

| Compound # | Structure |
|---|---|
| 101 | 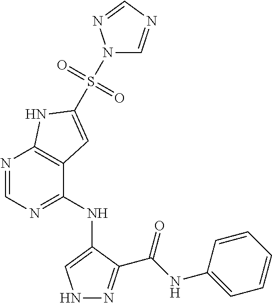 [SMILES: O=C(Nc1ccccc1)c1n[nH]cc1Nc1ncnc2[nH]c(S(=O)(=O)n3cncn3)cc12] |
| 102 | 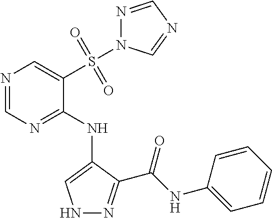 [SMILES: O=C(Nc1ccccc1)c1n[nH]cc1Nc1ncncc1S(=O)(=O)n1cncn1] |
| 103 | 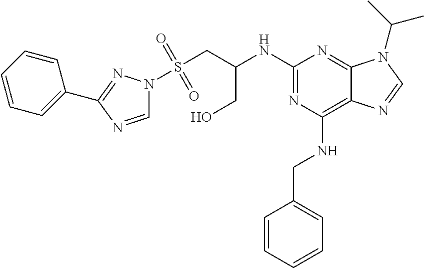 [SMILES: CC(C)n1cnc2c(NCc3ccccc3)nc(NC(CO)CS(=O)(=O)n3cnc(-c4ccccc4)n3)nc21] |
| 104 | 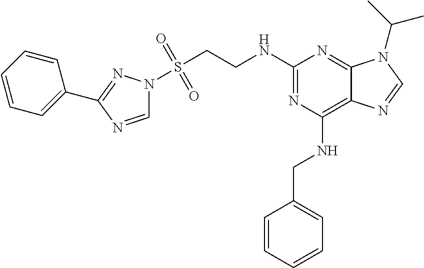 [SMILES: CC(C)n1cnc2c(NCc3ccccc3)nc(NCCS(=O)(=O)n3cnc(-c4ccccc4)n3)nc21] |
| 105 | 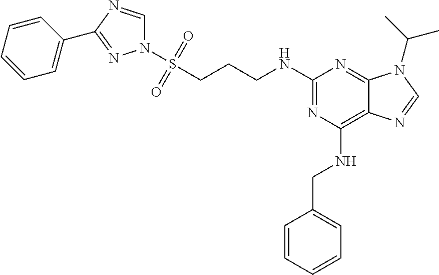 [SMILES: CC(C)n1cnc2c(NCc3ccccc3)nc(NCCCS(=O)(=O)n3cnc(-c4ccccc4)n3)nc21] |
| 106 | 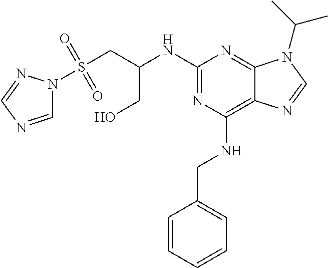 [SMILES: CC(C)n1cnc2c(NCc3ccccc3)nc(NC(CO)CS(=O)(=O)n3cncn3)nc21] |
| 107 | 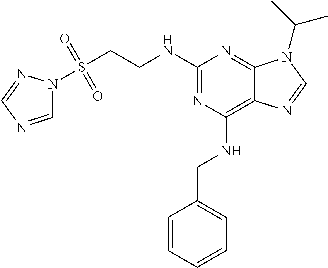 [SMILES: CC(C)n1cnc2c(NCc3ccccc3)nc(NCCS(=O)(=O)n3cncn3)nc21] |
| 108 | 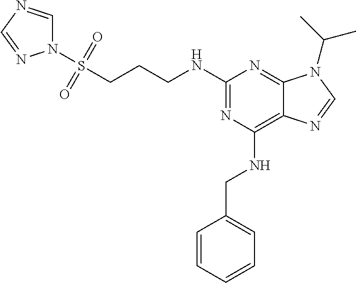 [SMILES: CC(C)n1cnc2c(NCc3ccccc3)nc(NCCCS(=O)(=O)n3cncn3)nc21] |
| 109 | 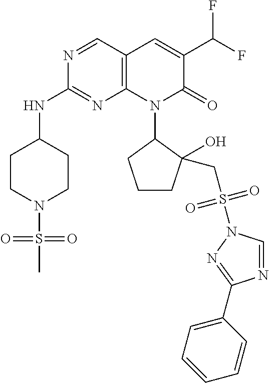 [SMILES: CS(=O)(=O)N1CCC(Nc2ncc3cc(C(F)F)c(=O)n(C4CCCC4(O)CS(=O)(=O)n4cnc(-c5ccccc5)n4)c3n2)CC1] |
| 110 | 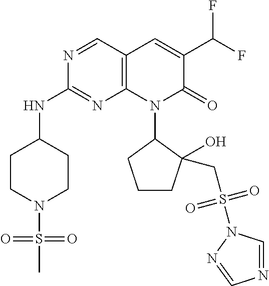 [SMILES: CS(=O)(=O)N1CCC(Nc2ncc3cc(C(F)F)c(=O)n(C4CCCC4(O)CS(=O)(=O)n4cncn4)c3n2)CC1] |
| 111 | 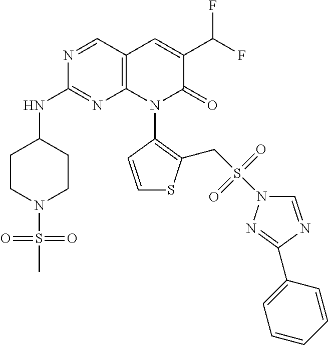 [SMILES: CS(=O)(=O)N1CCC(Nc2ncc3cc(C(F)F)c(=O)n(-c4ccsc4CS(=O)(=O)n4cnc(-c5ccccc5)n4)c3n2)CC1] |
| 112 | 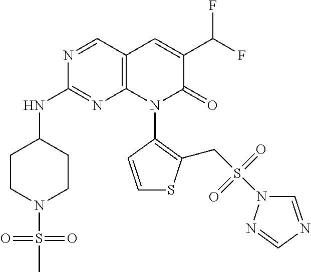 [SMILES: CS(=O)(=O)N1CCC(Nc2ncc3cc(C(F)F)c(=O)n(-c4ccsc4CS(=O)(=O)n4cncn4)c3n2)CC1] |
| 113 | 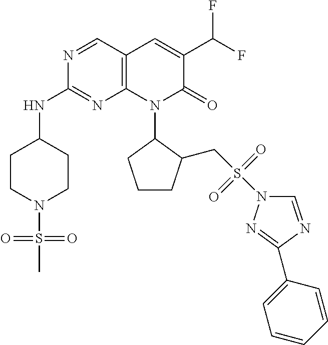 [SMILES: CS(=O)(=O)N1CCC(Nc2ncc3cc(C(F)F)c(=O)n(C4CCCC4CS(=O)(=O)n4cnc(-c5ccccc5)n4)c3n2)CC1] |
| 114 | 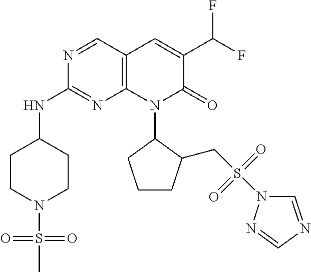 [SMILES: CS(=O)(=O)N1CCC(Nc2ncc3cc(C(F)F)c(=O)n(C4CCCC4CS(=O)(=O)n4cncn4)c3n2)CC1] |
| 115 | 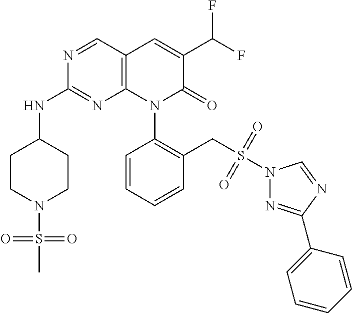 [SMILES: CS(=O)(=O)N1CCC(Nc2ncc3cc(C(F)F)c(=O)n(-c4ccccc4CS(=O)(=O)n4cnc(-c5ccccc5)n4)c3n2)CC1] |
| 116 | 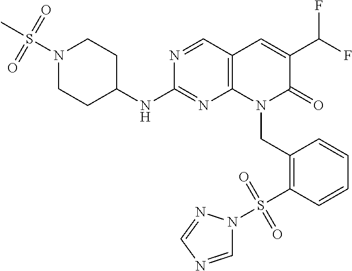 [SMILES: CS(=O)(=O)N1CCC(Nc2ncc3cc(C(F)F)c(=O)n(Cc4ccccc4S(=O)(=O)n4cncn4)c3n2)CC1] |
| 117 | 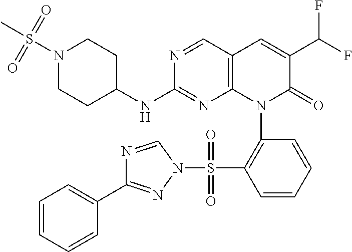 [SMILES: CS(=O)(=O)N1CCC(Nc2ncc3cc(C(F)F)c(=O)n(-c4ccccc4S(=O)(=O)n4cnc(-c5ccccc5)n4)c3n2)CC1] |
| 118 | 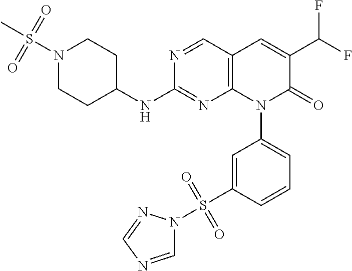 [SMILES: CS(=O)(=O)N1CCC(Nc2ncc3cc(C(F)F)c(=O)n(-c4cccc(S(=O)(=O)n5cncn5)c4)c3n2)CC1] |
| 119 | 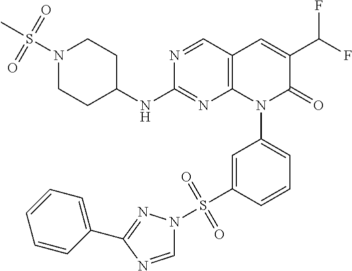 [SMILES: CS(=O)(=O)N1CCC(Nc2ncc3cc(C(F)F)c(=O)n(-c4cccc(S(=O)(=O)n5cnc(-c6ccccc6)n5)c4)c3n2)CC1] |
| 120 | 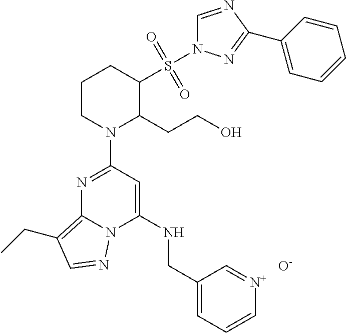 |
| 121 | 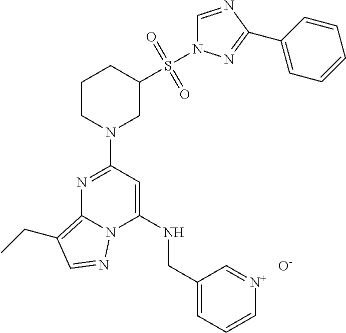 |
| 122 | 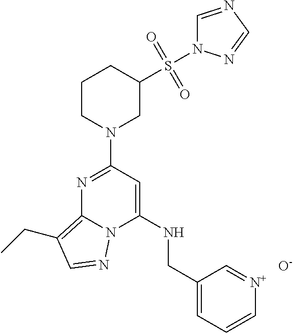 |
| 123 | 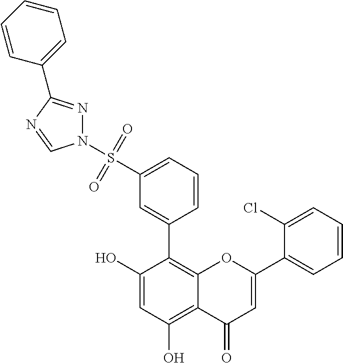 [SMILES: O=c1cc(-c2ccccc2Cl)oc2c(-c3cccc(S(=O)(=O)n4cnc(-c5ccccc5)n4)c3)c(O)cc(O)c12] |
| 124 | 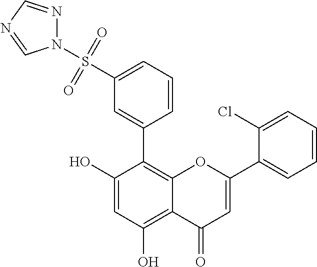 [SMILES: O=c1cc(-c2ccccc2Cl)oc2c(-c3cccc(S(=O)(=O)n4cncn4)c3)c(O)cc(O)c12] |
| 125 | 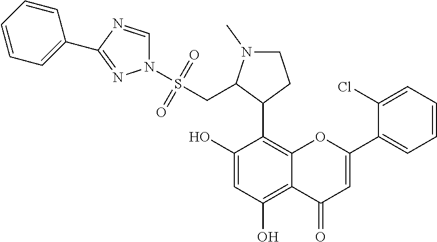 [SMILES: CN1CCC(c2c(O)cc(O)c3c(=O)cc(-c4ccccc4Cl)oc23)C1CS(=O)(=O)n1cnc(-c2ccccc2)n1] |
| 126 | 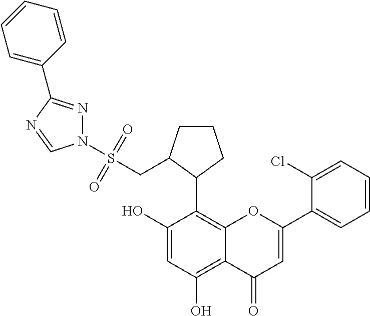 [SMILES: O=c1cc(-c2ccccc2Cl)oc2c(C3CCCC3CS(=O)(=O)n3cnc(-c4ccccc4)n3)c(O)cc(O)c12] |
| 127 | 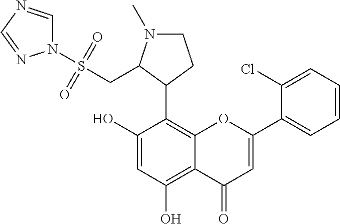 [SMILES: CN1CCC(c2c(O)cc(O)c3c(=O)cc(-c4ccccc4Cl)oc23)C1CS(=O)(=O)n1cncn1] |
| 128 | 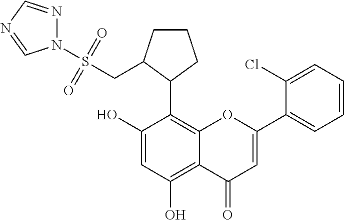 [SMILES: O=c1cc(-c2ccccc2Cl)oc2c(C3CCCC3CS(=O)(=O)n3cncn3)c(O)cc(O)c12] |
| 129 | 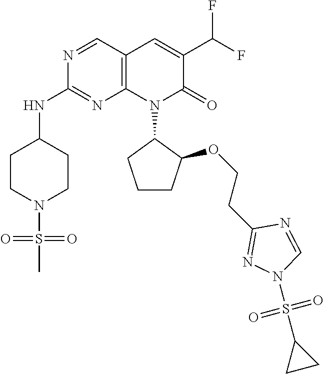 [SMILES: CS(=O)(=O)N1CCC(Nc2ncc3cc(C(F)F)c(=O)n([C@H]4CCC[C@@H]4OCCc4ncn(S(=O)(=O)C5CC5)n4)c3n2)CC1] |
| 130 | 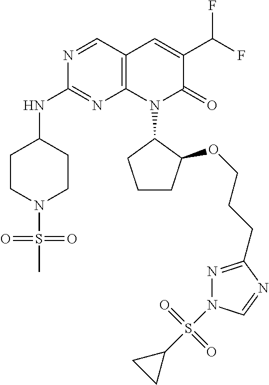 [SMILES: CS(=O)(=O)N1CCC(Nc2ncc3cc(C(F)F)c(=O)n([C@H]4CCC[C@@H]4OCCCc4ncn(S(=O)(=O)C5CC5)n4)c3n2)CC1] |
| 131 | 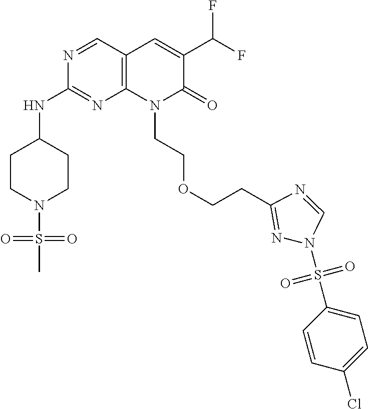 [SMILES: CS(=O)(=O)N1CCC(Nc2ncc3cc(C(F)F)c(=O)n(CCOCCc4ncn(S(=O)(=O)c5ccc(Cl)cc5)n4)c3n2)CC1] |
| 132 | 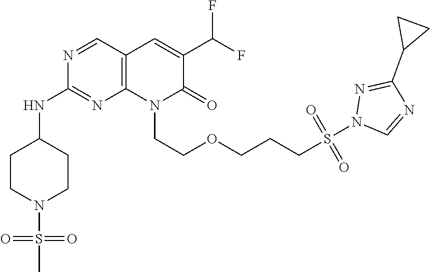 [SMILES: CS(=O)(=O)N1CCC(Nc2ncc3cc(C(F)F)c(=O)n(CCOCCCS(=O)(=O)n4cnc(C5CC5)n4)c3n2)CC1] |
| 133 | 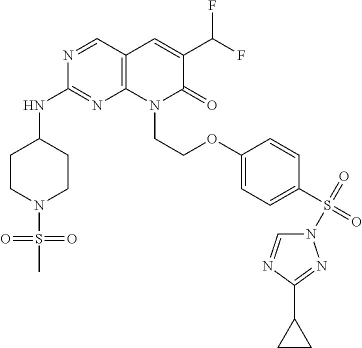 [SMILES: CS(=O)(=O)N1CCC(Nc2ncc3cc(C(F)F)c(=O)n(CCOc4ccc(S(=O)(=O)n5cnc(C6CC6)n5)cc4)c3n2)CC1] |
| 134 | 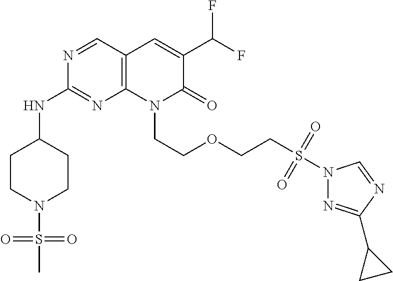 [SMILES: CS(=O)(=O)N1CCC(Nc2ncc3cc(C(F)F)c(=O)n(CCOCCS(=O)(=O)n4cnc(C5CC5)n4)c3n2)CC1] |
| 135 | 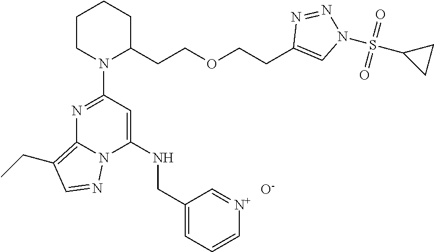 |
| 136 | 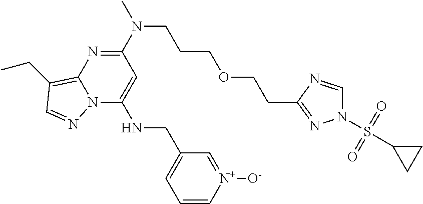 [SMILES: CCc1cnn2c(NCc3ccc[n+]([O-])c3)cc(N(C)CCCOCCc3ncn(S(=O)(=O)C4CC4)n3)nc12] |
| 137 | 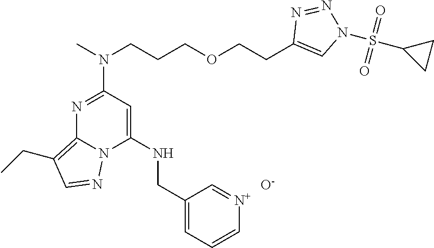 |
| 138 | 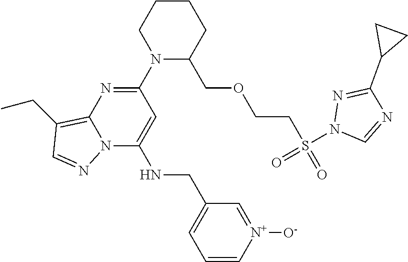 [SMILES: CCc1cnn2c(NCc3ccc[n+]([O-])c3)cc(N3CCCCC3COCCS(=O)(=O)n3cnc(C4CC4)n3)nc12] |
| 139 | 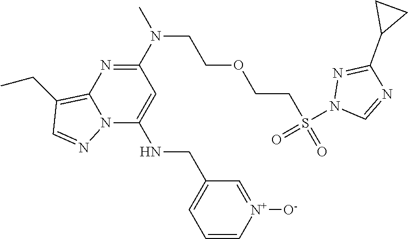 [SMILES: CCc1cnn2c(NCc3ccc[n+]([O-])c3)cc(N(C)CCOCCS(=O)(=O)n3cnc(C4CC4)n3)nc12] |
| 140 | 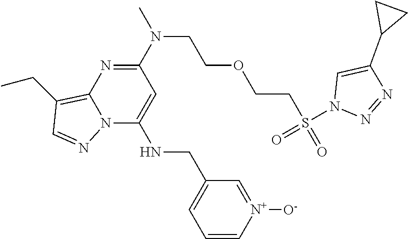 [SMILES: CCc1cnn2c(NCc3ccc[n+]([O-])c3)cc(N(C)CCOCCS(=O)(=O)n3cc(C4CC4)nn3)nc12] |
| 141 | 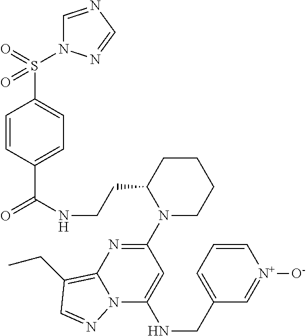 [SMILES: CCc1cnn2c(NCc3ccc[n+]([O-])c3)cc(N3CCCC[C@H]3CCNC(=O)c3ccc(S(=O)(=O)n4cncn4)cc3)nc12] |
| 142 | 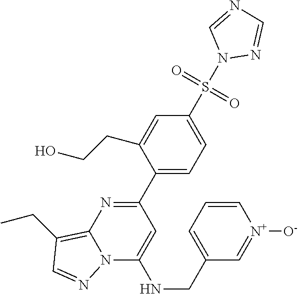 [SMILES: CCc1cnn2c(NCc3ccc[n+]([O-])c3)cc(-c3ccc(S(=O)(=O)n4cncn4)cc3CCO)nc12] |
| 143 | 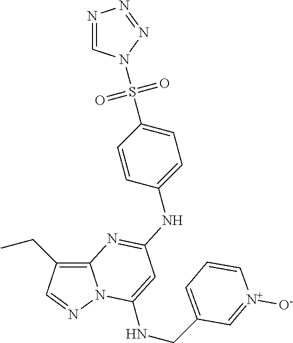 [SMILES: CCc1cnn2c(NCc3ccc[n+]([O-])c3)cc(Nc3ccc(S(=O)(=O)n4cnnn4)cc3)nc12] |
| 144 | 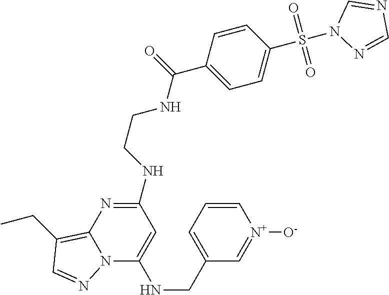 [SMILES: CCc1cnn2c(NCc3ccc[n+]([O-])c3)cc(NCCNC(=O)c3ccc(S(=O)(=O)n4cncn4)cc3)nc12] |
| 145 | 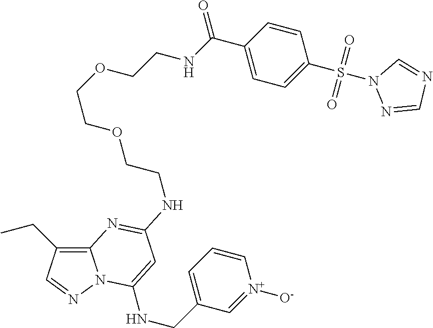 [SMILES: CCc1cnn2c(NCc3ccc[n+]([O-])c3)cc(NCCOCCOCCNC(=O)c3ccc(S(=O)(=O)n4cncn4)cc3)nc12] |
| 146 | 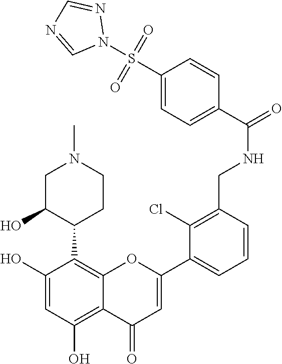 [SMILES: CN1CC[C@H](c2c(O)cc(O)c3c(=O)cc(-c4cccc(CNC(=O)c5ccc(S(=O)(=O)n6cncn6)cc5)c4Cl)oc23)[C@@H](O)C1] |
| 147 | 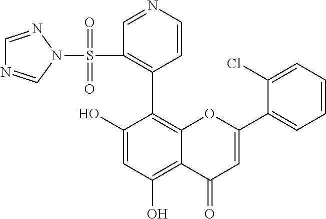 [SMILES: O=c1cc(-c2ccccc2Cl)oc2c(-c3ccncc3S(=O)(=O)n3cncn3)c(O)cc(O)c12] |
| 148 | 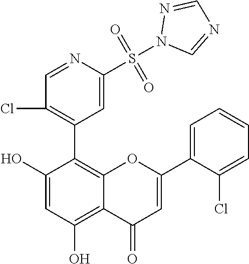 [SMILES: O=c1cc(-c2ccccc2Cl)oc2c(-c3cc(S(=O)(=O)n4cncn4)ncc3Cl)c(O)cc(O)c12] |
| 149 | 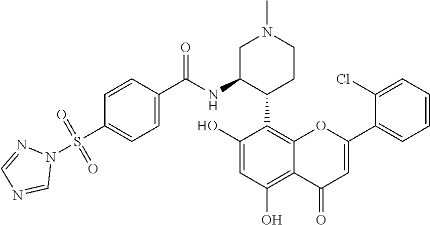 [SMILES: CN1CC[C@H](c2c(O)cc(O)c3c(=O)cc(-c4ccccc4Cl)oc23)[C@@H](NC(=O)c2ccc(S(=O)(=O)n3cncn3)cc2)C1] |
| 150 | 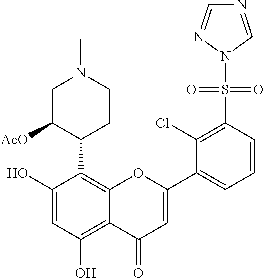 [SMILES: CC(=O)O[C@H]1CN(C)CC[C@@H]1c1c(O)cc(O)c2c(=O)cc(-c3cccc(S(=O)(=O)n4cncn4)c3Cl)oc12] |
| 151 | 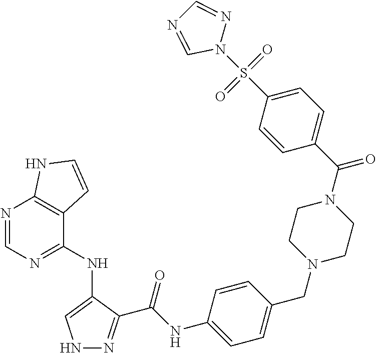 [SMILES: O=C(Nc1ccc(CN2CCN(C(=O)c3ccc(S(=O)(=O)n4cncn4)cc3)CC2)cc1)c1n[nH]cc1Nc1ncnc2[nH]ccc12] |
| 152 | 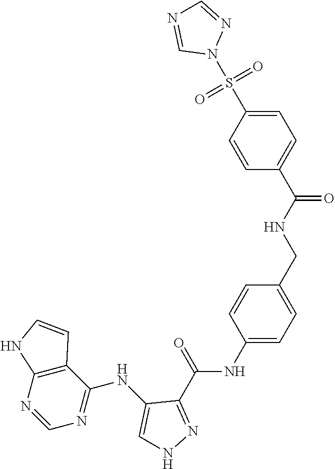 [SMILES: O=C(NCc1ccc(NC(=O)c2n[nH]cc2Nc2ncnc3[nH]ccc23)cc1)c1ccc(S(=O)(=O)n2cncn2)cc1] |
| 153 | 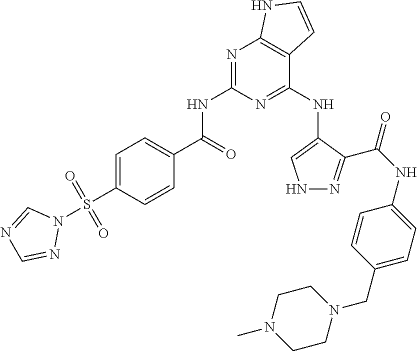 [SMILES: CN1CCN(Cc2ccc(NC(=O)c3n[nH]cc3Nc3nc(NC(=O)c4ccc(S(=O)(=O)n5cncn5)cc4)nc4[nH]ccc34)cc2)CC1] |
| 154 | 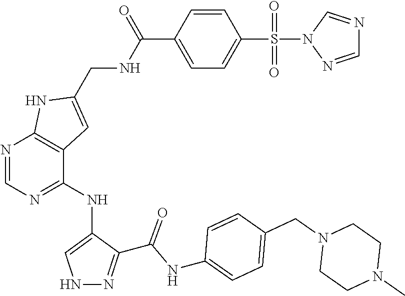 [SMILES: CN1CCN(Cc2ccc(NC(=O)c3n[nH]cc3Nc3ncnc4[nH]c(CNC(=O)c5ccc(S(=O)(=O)n6cncn6)cc5)cc34)cc2)CC1] |
| 155 | 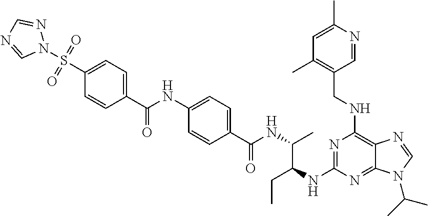 [SMILES: CC[C@H](Nc1nc(NCc2cnc(C)cc2C)c2ncn(C(C)C)c2n1)[C@@H](C)NC(=O)c1ccc(NC(=O)c2ccc(S(=O)(=O)n3cncn3)cc2)cc1] |
| 156 | 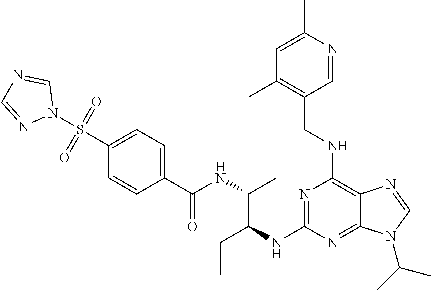 [SMILES: CC[C@H](Nc1nc(NCc2cnc(C)cc2C)c2ncn(C(C)C)c2n1)[C@@H](C)NC(=O)c1ccc(S(=O)(=O)n2cncn2)cc1] |
| 157 | 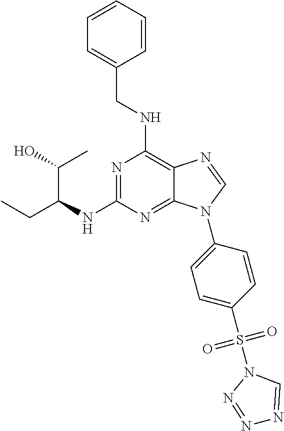 [SMILES: CC[C@H](Nc1nc(NCc2ccccc2)c2ncn(-c3ccc(S(=O)(=O)n4cnnn4)cc3)c2n1)[C@@H](C)O] |
| 158 | 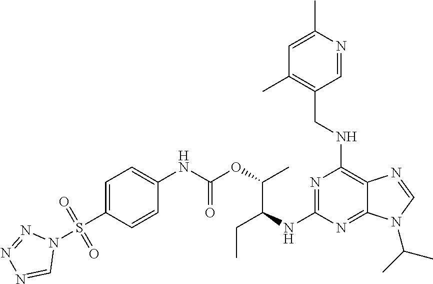 [SMILES: CC[C@H](Nc1nc(NCc2cnc(C)cc2C)c2ncn(C(C)C)c2n1)[C@@H](C)OC(=O)Nc1ccc(S(=O)(=O)n2cnnn2)cc1] |
| 159 | 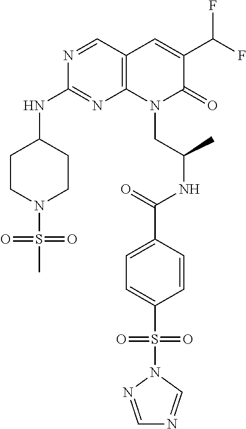 [SMILES: C[C@H](Cn1c(=O)c(C(F)F)cc2cnc(NC3CCN(S(C)(=O)=O)CC3)nc21)NC(=O)c1ccc(S(=O)(=O)n2cncn2)cc1] |
| 160 | 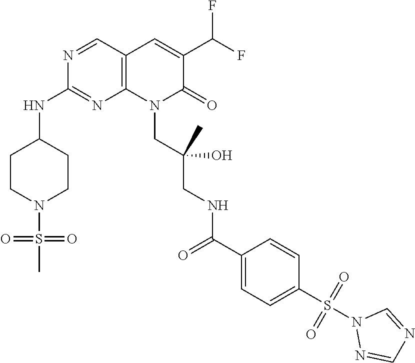 [SMILES: C[C@@](O)(CNC(=O)c1ccc(S(=O)(=O)n2cncn2)cc1)Cn1c(=O)c(C(F)F)cc2cnc(NC3CCN(S(C)(=O)=O)CC3)nc21] |
| 161 | 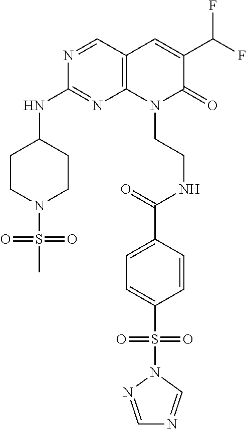 [SMILES: CS(=O)(=O)N1CCC(Nc2ncc3cc(C(F)F)c(=O)n(CCNC(=O)c4ccc(S(=O)(=O)n5cncn5)cc4)c3n2)CC1] |
| 162 | 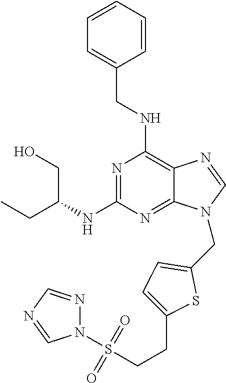 [SMILES: CC[C@H](CO)Nc1nc(NCc2ccccc2)c2ncn(Cc3ccc(CCS(=O)(=O)n4cncn4)s3)c2n1] |
| 163 | 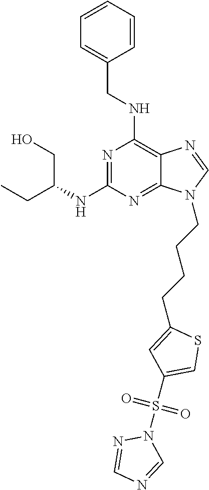 [SMILES: CC[C@H](CO)Nc1nc(NCc2ccccc2)c2ncn(CCCCc3cc(S(=O)(=O)n4cncn4)cs3)c2n1] |
| 164 | 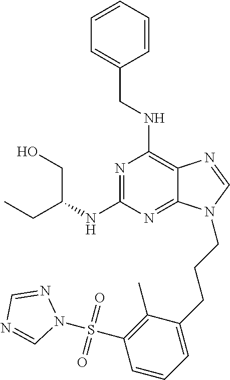 [SMILES: CC[C@H](CO)Nc1nc(NCc2ccccc2)c2ncn(CCCc3cccc(S(=O)(=O)n4cncn4)c3C)c2n1] |
| 165 | 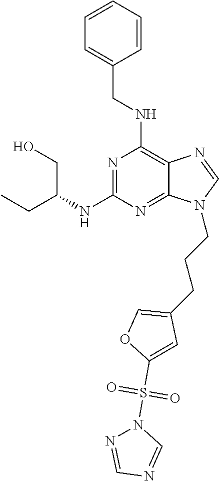 [SMILES: CC[C@H](CO)Nc1nc(NCc2ccccc2)c2ncn(CCCc3coc(S(=O)(=O)n4cncn4)c3)c2n1] |
| 166 | 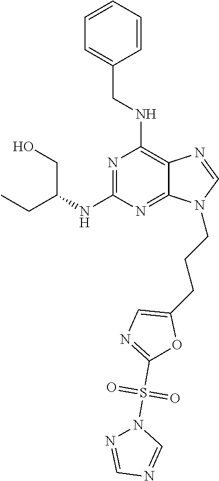 [SMILES: CC[C@H](CO)Nc1nc(NCc2ccccc2)c2ncn(CCCc3cnc(S(=O)(=O)n4cncn4)o3)c2n1] |
| 167 | 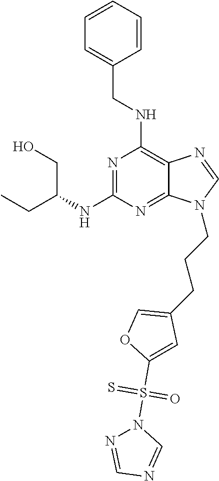 [SMILES: CC[C@H](CO)Nc1nc(NCc2ccccc2)c2ncn(CCCc3coc(S(=O)(=S)n4cncn4)c3)c2n1] |
| 168 | 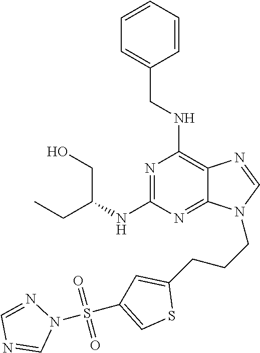 [SMILES: CC[C@H](CO)Nc1nc(NCc2ccccc2)c2ncn(CCCc3cc(S(=O)(=O)n4cncn4)cs3)c2n1] |
| 169 | 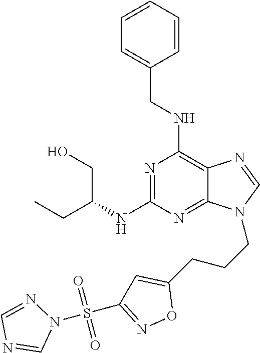 [SMILES: CC[C@H](CO)Nc1nc(NCc2ccccc2)c2ncn(CCCc3cc(S(=O)(=O)n4cncn4)no3)c2n1] |
| 170 | 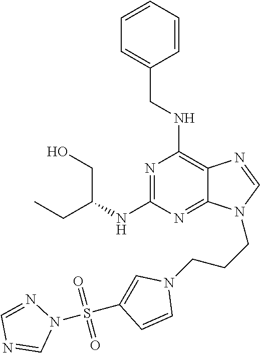 [SMILES: CC[C@H](CO)Nc1nc(NCc2ccccc2)c2ncn(CCCn3ccc(S(=O)(=O)n4cncn4)c3)c2n1] |
| 171 | 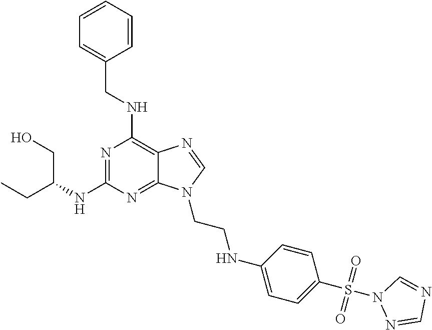 [SMILES: CC[C@H](CO)Nc1nc(NCc2ccccc2)c2ncn(CCNc3ccc(S(=O)(=O)n4cncn4)cc3)c2n1] |
| 172 | 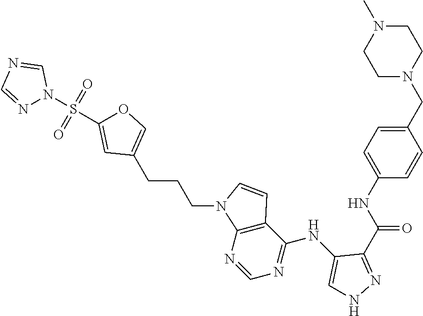 [SMILES: CN1CCN(Cc2ccc(NC(=O)c3n[nH]cc3Nc3ncnc4c3ccn4CCCc3coc(S(=O)(=O)n4cncn4)c3)cc2)CC1] |
| 173 | 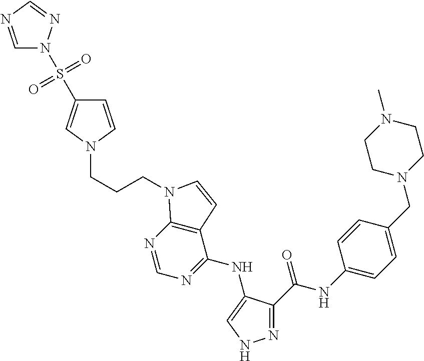 [SMILES: CN1CCN(Cc2ccc(NC(=O)c3n[nH]cc3Nc3ncnc4c3ccn4CCCn3ccc(S(=O)(=O)n4cncn4)c3)cc2)CC1] |
| 174 | 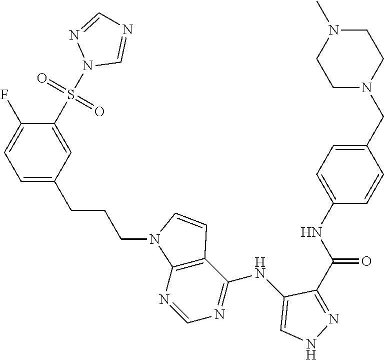 [SMILES: CN1CCN(Cc2ccc(NC(=O)c3n[nH]cc3Nc3ncnc4c3ccn4CCCc3ccc(F)c(S(=O)(=O)n4cncn4)c3)cc2)CC1] |
| 175 | 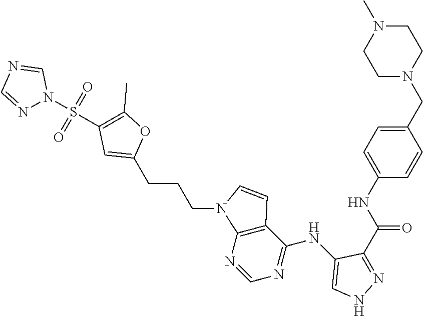 [SMILES: Cc1oc(CCCn2ccc3c(Nc4c[nH]nc4C(=O)Nc4ccc(CN5CCN(C)CC5)cc4)ncnc32)cc1S(=O)(=O)n1cncn1] |
| 176 | 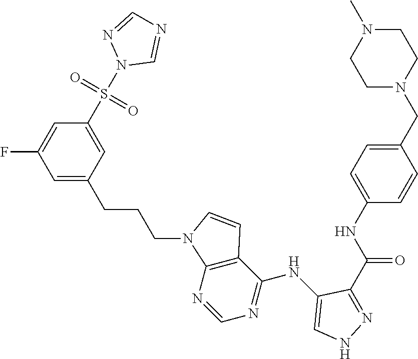 [SMILES: CN1CCN(Cc2ccc(NC(=O)c3n[nH]cc3Nc3ncnc4c3ccn4CCCc3cc(F)cc(S(=O)(=O)n4cncn4)c3)cc2)CC1] |
| 177 | 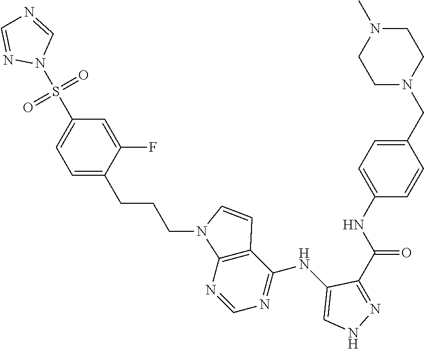 [SMILES: CN1CCN(Cc2ccc(NC(=O)c3n[nH]cc3Nc3ncnc4c3ccn4CCCc3ccc(S(=O)(=O)n4cncn4)cc3F)cc2)CC1] |
| 178 | 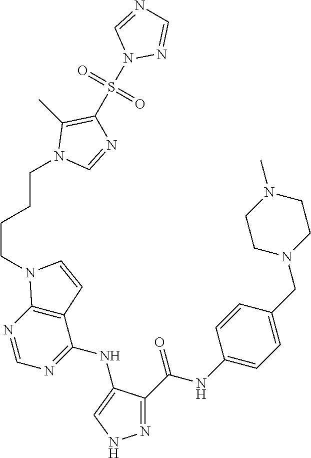 [SMILES: Cc1c(S(=O)(=O)n2cncn2)ncn1CCCCn1ccc2c(Nc3c[nH]nc3C(=O)Nc3ccc(CN4CCN(C)CC4)cc3)ncnc21] |
| 179 | 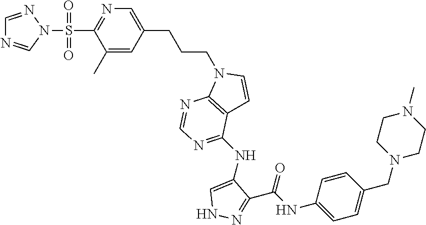 [SMILES: Cc1cc(CCCn2ccc3c(Nc4c[nH]nc4C(=O)Nc4ccc(CN5CCN(C)CC5)cc4)ncnc32)cnc1S(=O)(=O)n1cncn1] |
| 180 | 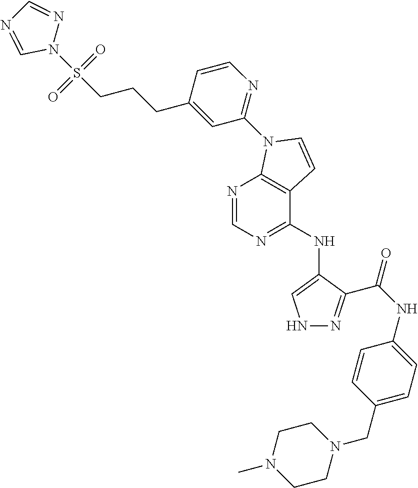 [SMILES: CN1CCN(Cc2ccc(NC(=O)c3n[nH]cc3Nc3ncnc4c3ccn4-c3cc(CCCS(=O)(=O)n4cncn4)ccn3)cc2)CC1] |
| 181 | 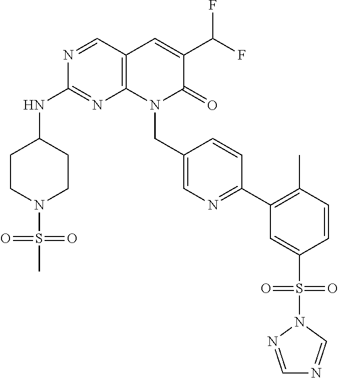 [SMILES: Cc1ccc(S(=O)(=O)n2cncn2)cc1-c1ccc(Cn2c(=O)c(C(F)F)cc3cnc(NC4CCN(S(C)(=O)=O)CC4)nc32)cn1] |
| 182 | 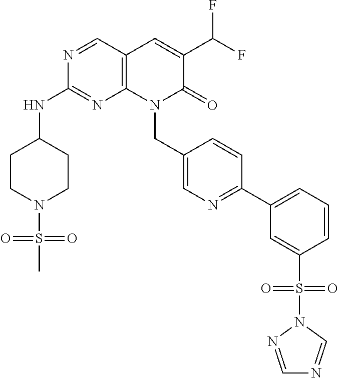 [SMILES: CS(=O)(=O)N1CCC(Nc2ncc3cc(C(F)F)c(=O)n(Cc4ccc(-c5cccc(S(=O)(=O)n6cncn6)c5)nc4)c3n2)CC1] |
| 183 | 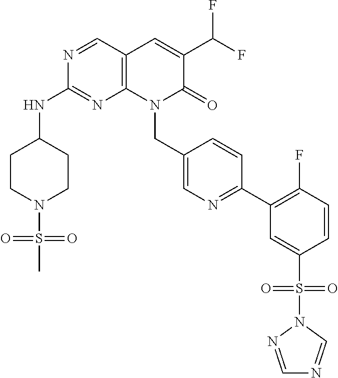 [SMILES: CS(=O)(=O)N1CCC(Nc2ncc3cc(C(F)F)c(=O)n(Cc4ccc(-c5cc(S(=O)(=O)n6cncn6)ccc5F)nc4)c3n2)CC1] |
| 184 | 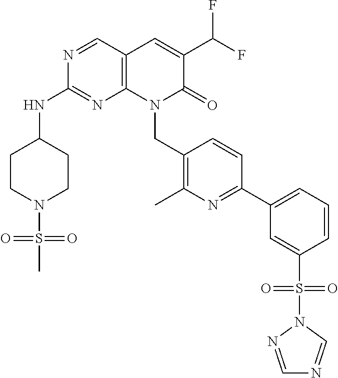 [SMILES: Cc1nc(-c2cccc(S(=O)(=O)n3cncn3)c2)ccc1Cn1c(=O)c(C(F)F)cc2cnc(NC3CCN(S(C)(=O)=O)CC3)nc21] |
| 185 | 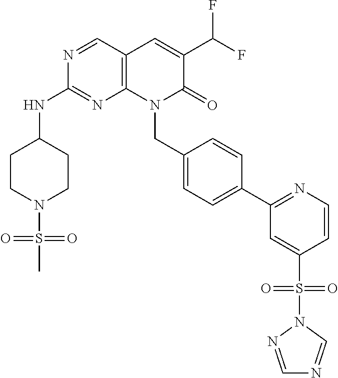 [SMILES: CS(=O)(=O)N1CCC(Nc2ncc3cc(C(F)F)c(=O)n(Cc4ccc(-c5cc(S(=O)(=O)n6cncn6)ccn5)cc4)c3n2)CC1] |
| 186 | 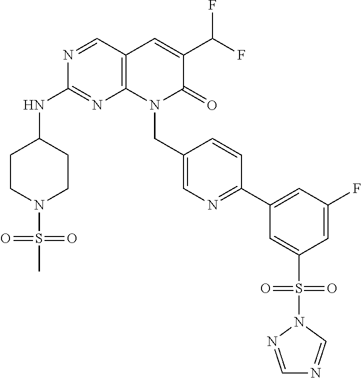 [SMILES: CS(=O)(=O)N1CCC(Nc2ncc3cc(C(F)F)c(=O)n(Cc4ccc(-c5cc(F)cc(S(=O)(=O)n6cncn6)c5)nc4)c3n2)CC1] |
| 187 | 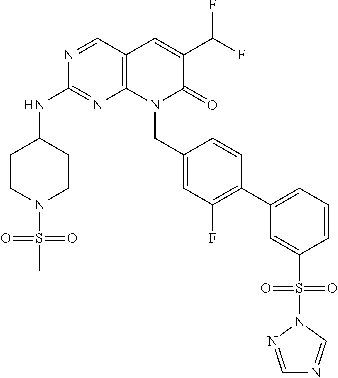 [SMILES: CS(=O)(=O)N1CCC(Nc2ncc3cc(C(F)F)c(=O)n(Cc4ccc(-c5cccc(S(=O)(=O)n6cncn6)c5)c(F)c4)c3n2)CC1] |
| 188 | 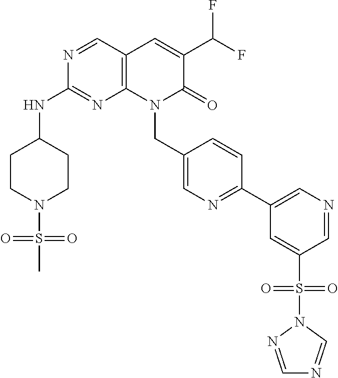 [SMILES: CS(=O)(=O)N1CCC(Nc2ncc3cc(C(F)F)c(=O)n(Cc4ccc(-c5cncc(S(=O)(=O)n6cncn6)c5)nc4)c3n2)CC1] |
| 189 | 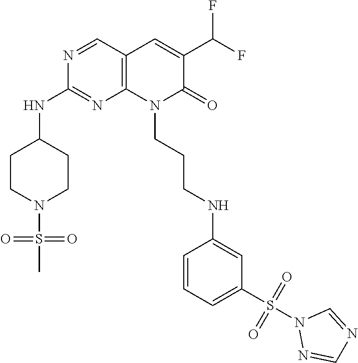 [SMILES: CS(=O)(=O)N1CCC(Nc2ncc3cc(C(F)F)c(=O)n(CCCNc4cccc(S(=O)(=O)n5cncn5)c4)c3n2)CC1] |
| 190 | 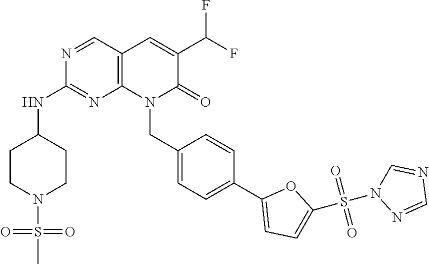 [SMILES: CS(=O)(=O)N1CCC(Nc2ncc3cc(C(F)F)c(=O)n(Cc4ccc(-c5ccc(S(=O)(=O)n6cncn6)o5)cc4)c3n2)CC1] |
| 191 | 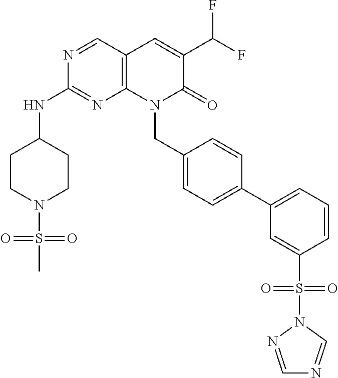 [SMILES: CS(=O)(=O)N1CCC(Nc2ncc3cc(C(F)F)c(=O)n(Cc4ccc(-c5cccc(S(=O)(=O)n6cncn6)c5)cc4)c3n2)CC1] |
| 192 | 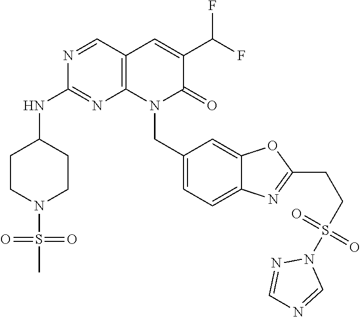 [SMILES: CS(=O)(=O)N1CCC(Nc2ncc3cc(C(F)F)c(=O)n(Cc4ccc5nc(CCS(=O)(=O)n6cncn6)oc5c4)c3n2)CC1] |
| 193 | 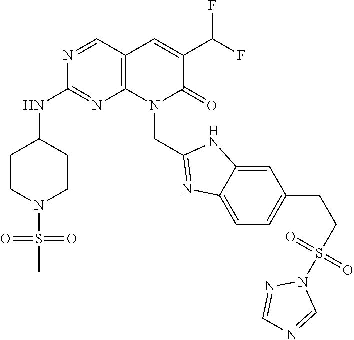 [SMILES: CS(=O)(=O)N1CCC(Nc2ncc3cc(C(F)F)c(=O)n(Cc4nc5ccc(CCS(=O)(=O)n6cncn6)cc5[nH]4)c3n2)CC1] |
| 194 | 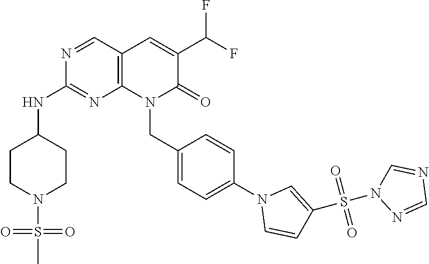 [SMILES: CS(=O)(=O)N1CCC(Nc2ncc3cc(C(F)F)c(=O)n(Cc4ccc(-n5ccc(S(=O)(=O)n6cncn6)c5)cc4)c3n2)CC1] |
| 195 | 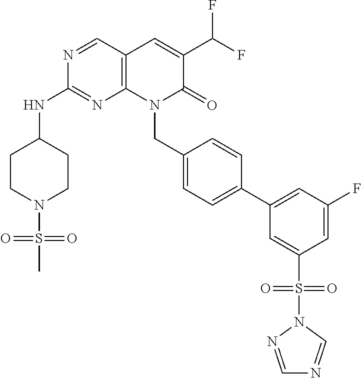 [SMILES: CS(=O)(=O)N1CCC(Nc2ncc3cc(C(F)F)c(=O)n(Cc4ccc(-c5cc(F)cc(S(=O)(=O)n6cncn6)c5)cc4)c3n2)CC1] |
| 196 | 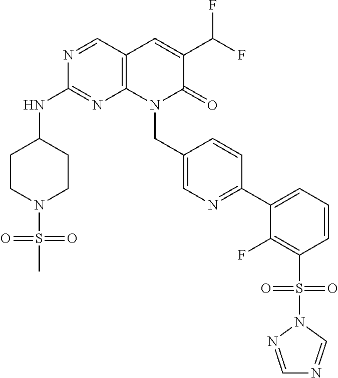 [SMILES: CS(=O)(=O)N1CCC(Nc2ncc3cc(C(F)F)c(=O)n(Cc4ccc(-c5cccc(S(=O)(=O)n6cncn6)c5F)nc4)c3n2)CC1] |
| 197 | 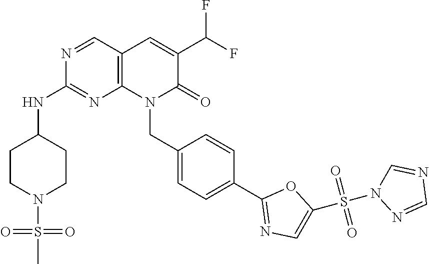 [SMILES: CS(=O)(=O)N1CCC(Nc2ncc3cc(C(F)F)c(=O)n(Cc4ccc(-c5ncc(S(=O)(=O)n6cncn6)o5)cc4)c3n2)CC1] |
| 198 | 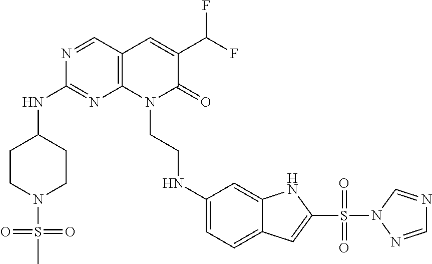 [SMILES: CS(=O)(=O)N1CCC(Nc2ncc3cc(C(F)F)c(=O)n(CCNc4ccc5cc(S(=O)(=O)n6cncn6)[nH]c5c4)c3n2)CC1] |
| 199 | 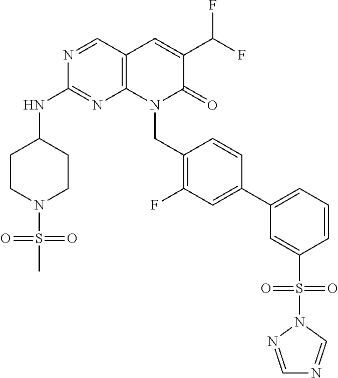 [SMILES: CS(=O)(=O)N1CCC(Nc2ncc3cc(C(F)F)c(=O)n(Cc4ccc(-c5cccc(S(=O)(=O)n6cncn6)c5)cc4F)c3n2)CC1] |
| 200 | 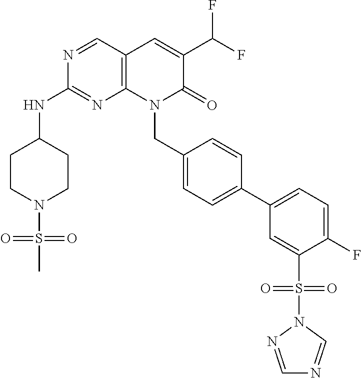 [SMILES: CS(=O)(=O)N1CCC(Nc2ncc3cc(C(F)F)c(=O)n(Cc4ccc(-c5ccc(F)c(S(=O)(=O)n6cncn6)c5)cc4)c3n2)CC1] |
| 201 | 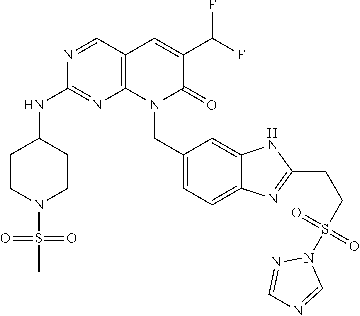 [SMILES: CS(=O)(=O)N1CCC(Nc2ncc3cc(C(F)F)c(=O)n(Cc4ccc5nc(CCS(=O)(=O)n6cncn6)[nH]c5c4)c3n2)CC1] |
| 202 | 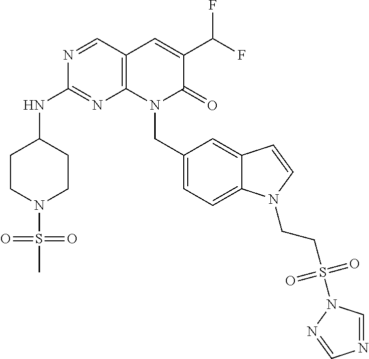 [SMILES: CS(=O)(=O)N1CCC(Nc2ncc3cc(C(F)F)c(=O)n(Cc4ccc5c(ccn5CCS(=O)(=O)n5cncn5)c4)c3n2)CC1] |
| 203 | 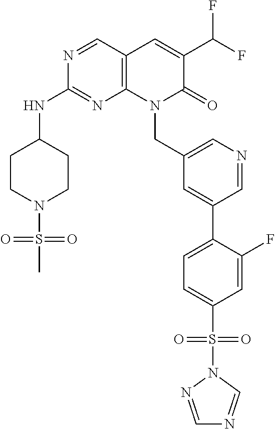 [SMILES: CS(=O)(=O)N1CCC(Nc2ncc3cc(C(F)F)c(=O)n(Cc4cncc(-c5ccc(S(=O)(=O)n6cncn6)cc5F)c4)c3n2)CC1] |
| 204 | 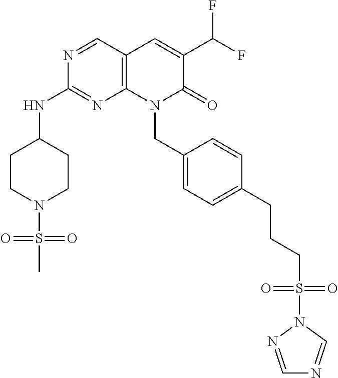 [SMILES: CS(=O)(=O)N1CCC(Nc2ncc3cc(C(F)F)c(=O)n(Cc4ccc(CCCS(=O)(=O)n5cncn5)cc4)c3n2)CC1] |
| 205 | 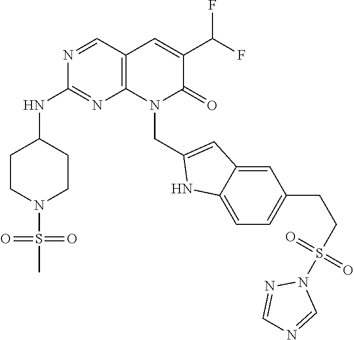 [SMILES: CS(=O)(=O)N1CCC(Nc2ncc3cc(C(F)F)c(=O)n(Cc4cc5cc(CCS(=O)(=O)n6cncn6)ccc5[nH]4)c3n2)CC1] |
| 206 | 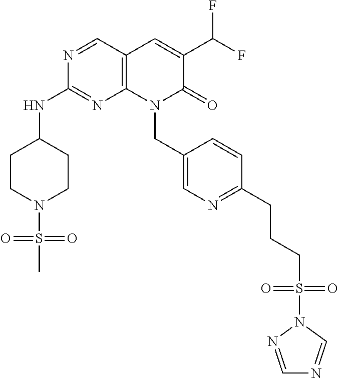 [SMILES: CS(=O)(=O)N1CCC(Nc2ncc3cc(C(F)F)c(=O)n(Cc4ccc(CCCS(=O)(=O)n5cncn5)nc4)c3n2)CC1] |
| 207 | 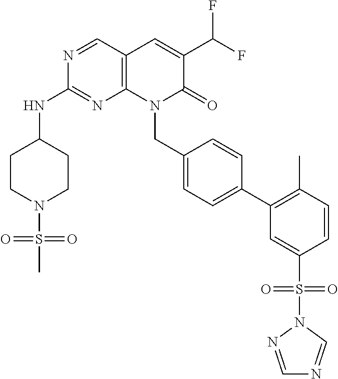 [SMILES: Cc1ccc(S(=O)(=O)n2cncn2)cc1-c1ccc(Cn2c(=O)c(C(F)F)cc3cnc(NC4CCN(S(C)(=O)=O)CC4)nc32)cc1] |
| 208 | 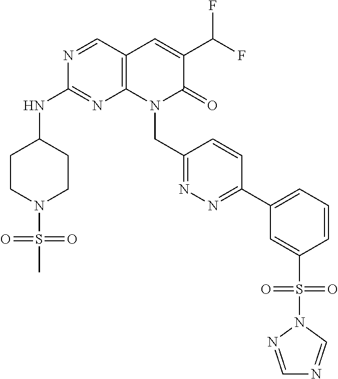 [SMILES: CS(=O)(=O)N1CCC(Nc2ncc3cc(C(F)F)c(=O)n(Cc4ccc(-c5cccc(S(=O)(=O)n6cncn6)c5)nn4)c3n2)CC1] |
| 209 | 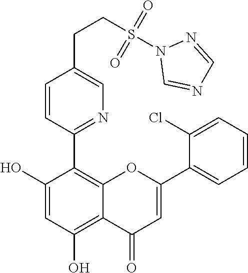 [SMILES: O=c1cc(-c2ccccc2Cl)oc2c(-c3ccc(CCS(=O)(=O)n4cncn4)cn3)c(O)cc(O)c12] |
| 210 | 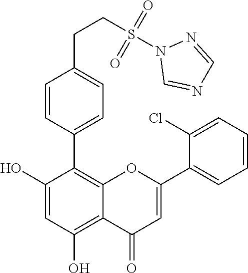 [SMILES: O=c1cc(-c2ccccc2Cl)oc2c(-c3ccc(CCS(=O)(=O)n4cncn4)cc3)c(O)cc(O)c12] |
| 211 | 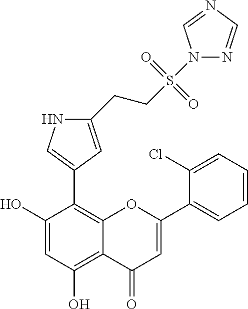 [SMILES: O=c1cc(-c2ccccc2Cl)oc2c(-c3c[nH]c(CCS(=O)(=O)n4cncn4)c3)c(O)cc(O)c12] |
| 212 | 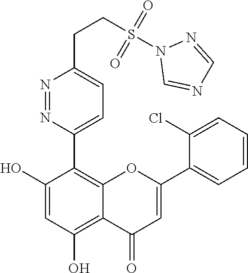 [SMILES: O=c1cc(-c2ccccc2Cl)oc2c(-c3ccc(CCS(=O)(=O)n4cncn4)nn3)c(O)cc(O)c12] |
| 213 | 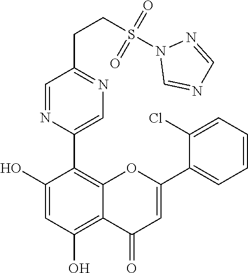 [SMILES: O=c1cc(-c2ccccc2Cl)oc2c(-c3cnc(CCS(=O)(=O)n4cncn4)cn3)c(O)cc(O)c12] |
| 214 | 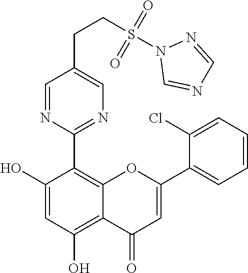 [SMILES: O=c1cc(-c2ccccc2Cl)oc2c(-c3ncc(CCS(=O)(=O)n4cncn4)cn3)c(O)cc(O)c12] |
| 215 | 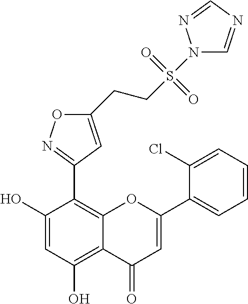 [SMILES: O=c1cc(-c2ccccc2Cl)oc2c(-c3cc(CCS(=O)(=O)n4cncn4)on3)c(O)cc(O)c12] |
| 216 | 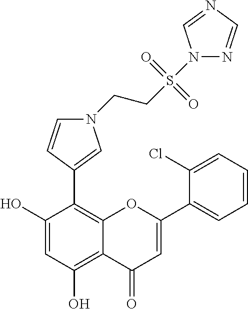 [SMILES: O=c1cc(-c2ccccc2Cl)oc2c(-c3ccn(CCS(=O)(=O)n4cncn4)c3)c(O)cc(O)c12] |
| 217 | 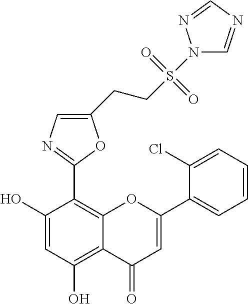 [SMILES: O=c1cc(-c2ccccc2Cl)oc2c(-c3ncc(CCS(=O)(=O)n4cncn4)o3)c(O)cc(O)c12] |
| 218 | 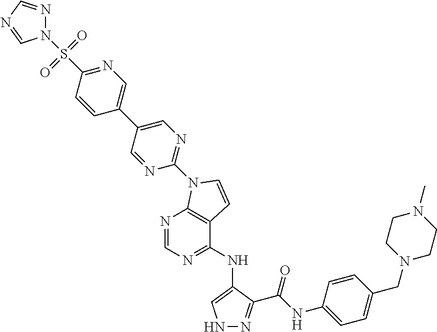 [SMILES: CN1CCN(Cc2ccc(NC(=O)c3n[nH]cc3Nc3ncnc4c3ccn4-c3ncc(-c4ccc(S(=O)(=O)n5cncn5)nc4)cn3)cc2)CC1] |
| 219 | 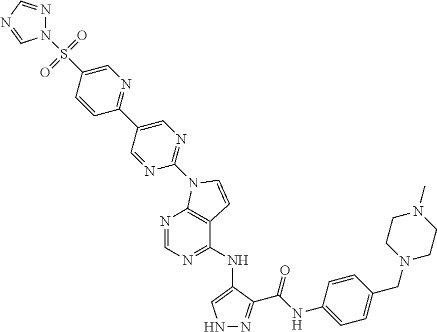 [SMILES: CN1CCN(Cc2ccc(NC(=O)c3n[nH]cc3Nc3ncnc4c3ccn4-c3ncc(-c4ccc(S(=O)(=O)n5cncn5)cn4)cn3)cc2)CC1] |
| 220 | 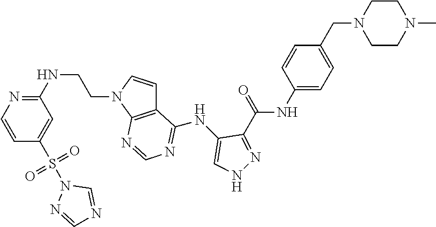 [SMILES: CN1CCN(Cc2ccc(NC(=O)c3n[nH]cc3Nc3ncnc4c3ccn4CCNc3cc(S(=O)(=O)n4cncn4)ccn3)cc2)CC1] |
| 221 | 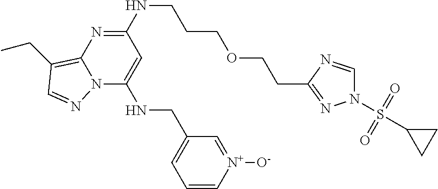 [SMILES: CCc1cnn2c(NCc3ccc[n+]([O-])c3)cc(NCCCOCCc3ncn(S(=O)(=O)C4CC4)n3)nc12] |
| 222 | 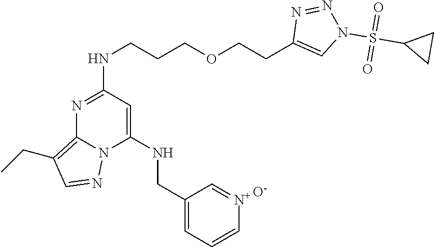 |
| 223 | 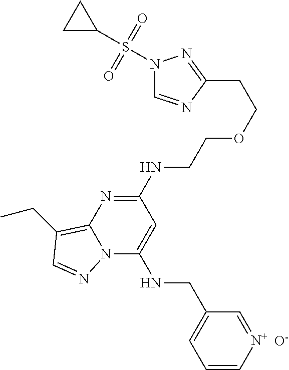 |
| 224 | 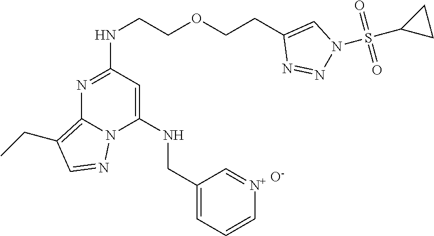 |

### Biological Assays

### Cdk2 Enzymatic TR-FRET Assay

A TR-FRET system was used to detect CycE1-CDK2 kinase activity. The assay used a Europium-labeled anti-phospho-Ser/Thr antibody and ULight™-labeled peptide substrate (eIF4E-binding protein 1). When CycE1-CDK2 phosphorylates the peptide the phosphorylated Thr is recognized by the Europium-labeled anti-phospho-Ser/Thr antibody. Upon excitation of the Europium donor fluorophore at 337 nm, energy is transferred to the ULight™ acceptor dye on the substrate, resulting in the emission of light at 665 nm. The fluorescent signal is proportional to the level of ULight™ peptide phosphorylation and to the kinase activity.

Materials

| Reagent | Type | Provider |
|---|---|---|
| CDK2-CycE | Kinase | Carna Bioscience |
| ULight-eIF4E-BP peptide | Substrate | PerkinElmer |
| Ultra-Eu-anti-P-eIF4E | Antibody | PerkinElmer |
| ATP | Substrate | Sigma |
| EDTA | Stop reagent | Sigma |
| LANCE Detection Buffer | Buffer | PerkinElmer |
| JNJ7706621 | Inhibitor | MCE |
| Proxiplate-384 PLUS/200W | Plate | PerkinElmer |

Instrumentation

The experiments were performed by using a PHERAstar FSX plate reader (BMG LABTECH), a Hamilton Microlab STAR (Hamilton), a CyBio Felix anda CyBio Well (CyBio).

Buffers and Solutions

The stock solution was prepared as follows:
1× Reaction buffer: 50 mM HEPES pH 7.5, 10 mM MgCl₂, 0.5 mM TCEP, 0.01% Tween®20, 1 mM EGTA, 0.001% fatty acid free (faf)-BSA;
1× Detection buffer: equilibrate the Detection buffer at RT for at least 20 minutes then dilute 10-fold the stock in water;
4×ENZYME mix: CDK2/CycE enzyme (5 nM final concentration in kinase reaction) in 1× Reaction buffer;
4×SUBSTRATE mix: 4E-BP substrate (50 nM final concentration in kinase reaction)+ATP (100 μM final concentration in kinase reaction) in 1× Reaction buffer
2×STOP/DETECTION mix: EDTA (10 mM final concentration in assay)+Anti-Phosphate antibody (0.5 nM final concentration in assay) in 1× Detection buffer
Neutral Control: Assay with no inhibition (MAX activity)
Inhibitor Control: Assay with IC₁₀₀ (1 mM) reference inhibitor JNJ7706621 (MIN activity)

Analytical Methods

The assay was assembled directly in a white, small volume, Proxiplate (PerkinElmer) as follows:
Step 1: Charge compounds=5 μL/well
Step 2: Charge kinase=2.5 μL/well→total volume 7.5 μL/well
   The plate is sealed and incubated at room temperature for 60 minutes
Step 3: Substrate mix (substrate/ATP)=2.5 μL/well→total enzymatic reaction volume 10 μL/well
   The plate is sealed and incubated at room temperature for 60 minutes
Step 4: Stop/Detection mix (EDTA/antibody)=10 μL/well→total volume 20 μL/well
   The plate is sealed and incubated at room temperature for at least 2 hours and fluorescence signal is measured at PHERAstar FSX
      The reading conditions are the followings for PHERAstar FSX:

| | | |
|---|---|---|
| Optic module: | HTRF 337/665/620 | nm |
| Excitation filter: | 337 | nm |
| Emission filter: | 665 | nm |
| Integration | 60/100 | μs |
| start/time: | | |

Compound Handling and Preparation of Dose Response Curve

Test compounds are prepared as follow:

Test compounds are received in 96 Matrix rack in powder and dissolved in 100% DMSO at 10 mM concentration.

A 384-microtiter plate (MTP), containing 20 test compounds in eight-points dose response, is prepared. Test compounds dose response curves are prepared in automation in 100% DMSO in a CyBio Felix, starting from a DMSO stock solution of 10 mM. To obtain the eight-points dose-response curve, a serial dilution protocol in 100% DMSO is performed for each compound by using a 96 MTP as a support. 20 μL of the 10 mM stock solutions are transferred in row A of a 96 MTP U-bottom from column 2 to 11 for a total of 10 compounds/plate. Starting from the 10 mM stock, serial dilutions across rows, from A to B and proceeding up to H, are performed for each compound by transferring 6.3 μL of the more concentrated fluid in 13.7 μL of 100% DMSO.

In order to generate duplicate data points each well of the 96 MTP is then replicated into two wells of a 384 MTP plate for each compound and for each concentration to be tested. The 384 MTP compound plate is used as source plate in a “mother to child” process with a CyBi®-Well dispenser in which 1 μL of compound is moved into a destination plate pre-filled with 24 μL of reaction buffer, obtaining 2× concentrated compound solution.

Data Analysis

Genedata Screener® 17.0 was used for data analysis. In the implemented workflow, the following calculations were conducted:
Calculate the Response Value (KRV) from the respective original data as follow:

HTRF Assay: Ratio=665 nm_FRET/620 nm_Ab*10000
Calculate the % Inhibition of the compound (Inhibitory effect). KRV was normalized versus Neutral Controls and Inhibitor Controls (JNJ7706621) to obtain Activity % according to the following formula:

Activity %=−100*(x−<Neutral Control>)/(<Inhibitor Control>−<Neutral Control>)

Where < > indicate KRV median values by plates of the selected control types.

Note that applying the above formula Activity % values in inhibition mode are expressed with negative values, therefore a compound showing a complete inhibition of the target will have a value of −100.
Calculate the Robust Z Prime Factor (RZ′ Factor) as follows:

RZ′=1−(3*(RSD_{Neutral Controls}+RSD_{Inhibitor Controls})/Abs(<Neutral Controls>−<Inhibitor Controls>))

RZ′ factor is based on the same formulas as the Z′ factor (Zhang et al., J. of Biomol. Screen., 4(2):67-73, 1999), but standard deviations and means are replaced by the robust standard deviations (RSD) and medians (< >), respectively.
Perform compound curve fitting profile on each dose-response with the Analyzer module of Genedata Screener 16.0 based on Activity % values.

Curve Fitting Dose-Response Data

The curve fitting of each dose-response curve was then performed in the Analyzer module of Screener® on Activity [%].

This procedure was used to generate the CDK2 IC₅₀ data shown in Table 2 below.

### Cdk2 JUMP Dilution Assay

A JUMP dilution protocol can be used to evaluate the reversibility of inhibition of CycE1-CDK2 kinase activity by test compounds. To achieve full inhibition of the kinase activity, highly concentrated enzyme (100-fold the working concentration) is incubated for 180 minutes with 10-fold IC₅₀ of compound. The mixture is then diluted 100 times in buffer and the recovery of the enzyme activity is monitored with the LANCE™ Ultra Kinase Assay.

Materials

| Reagent | Type | Provider |
|---|---|---|
| CDK2-CycE | Kinase | Carna Bioscience |
| ULight-eIF4E-BP | Substrate | PerkinElmer |
| peptide | | |
| Ultra-Eu-anti-P-eIF4E | Antibody | PerkinElmer |
| ATP | Substrate | Sigma |
| EDTA | Stop reagent | Sigma |
| LANCE Detection Buffer | Buffer | PerkinElmer |
| JNJ7706621 | Inhibitor | MCE |
| Proxiplate-384 PLUS/200W | Plate | PerkinElmer |

Instrumentation

The experiments are performed by using a PHERAstar FSX plate reader (BMG LABTECH, a Hamilton Microlab STAR (Hamilton), a CyBio Felix, and a CyBio Well (CyBio).

Buffers and Solutions

Prepare stock solutions as follows:
1× Reaction buffer: 50 mM HEPES pH 7.5, 10 mM MgCl₂, 0.5 mM TCEP, 0.01% Tween®20, 1 mM EGTA, 0.001% fatty acid free (faf)-BSA
1× Detection buffer: equilibrate the Detection buffer at RT for at least 20 minutes then dilute 10-fold the stock in water
2×ASSAY BUFFER with DMSO: to mimic the final 2% DMSO present in the standard kinase assay protocol
20× IC₅₀ COMPOUNDS: fresh compounds are prepared from the 10 mM stock solution at a concentration equivalent to twenty-times of the previously calculated IC₅₀ value
200×ENZYME: CDK2/CycE enzyme in 1× Reaction buffer (1000 nM)
JUMP MIX: 10× IC₅₀ compounds and 100×ENZYME (500 nM) obtained mixing the 20× IC₅₀ COMPOUNDS together with the 200×ENZYME
POSITIVE CONTROL: to mimic 100% activity restored (DMSO only, treated as JUMP mix)
NEGATIVE CONTROL: to mimic 0% recovery (full inhibition: JUMP dilution 1:100 in assay buffer+1 μM (IC₁₀₀) JNJ7706621)
4×SUBSTRATE/DETECTION mix: 4E-BP substrate (50 nM final concentration in kinase reaction)+ATP (100 μM final concentration in kinase reaction)+Anti-Phosphate antibody (0.5 nM final concentration in assay) in 1× Detection buffer
2×STOP mix: EDTA (10 mM final concentration in assay)

Assay Protocol

The assay is assembled first in 96 well plate where compounds are incubated for 180 minutes in the presence of the concentrated enzyme and then JUMP diluted in reaction buffer. JUMP mix is then immediately transferred in 384 well plate (Proxiplate—PerkinElmer) and a standard enzymatic reaction is assembled in quadruplicate data points.

### Jump Dilution (in 96 Well Plate)

Step 1: 20× IC₅₀ compounds=5 μL/well [symbol]
Step 2: 200×ENZYME=5 μL/well [symbol] total volume of the JUMP mix 10 μL/well

The JUMP mix plate (10×IC₅₀ compounds and 100×ENZYME) is sealed and incubated at room temperature for 180 minutes

Step 3: JUMP dilution: the JUMP mix in 96 well plate is diluted 25-fold adding 240 μL/well of reaction buffer to generate a 4×ENZYME/JUMP mix to be used in standard kinase reaction

### Standard Kinase Reaction (in 384 Well Plate).

Immediately after JUMP dilution (Step 3) a standard kinase reaction is assembled as follows:

Step 4: Buffer and controls=5 μL/well
Step 5: 4×SUBSTRATE/DETECTION mix (substrate/ATP/antibody)=2.5 μL/well
Step 6: 4×ENZYME/JUMP mix (from JUMP dilution)=2.5 μL/well total enzymatic reaction volume 10 μL/well [symbol]
The plate is incubated at RT for 60 minutes.
   Step 7: 2×STOP mix (EDTA)=10 μL/well [symbol] total volume 20 μL/well
The plate is incubated at RT for at least 2 hours and fluorescence signal is measured at PHERAstar FSX.

The reading conditions are the followings for PHERAstar FSX:

| | | |
|---|---|---|
| Optic module: | HTRF 337/665/620 | nm |
| Excitation filter | 337 | nm |
| Emission filter: | 665 | nm |
| Integration | 60/100 | μs |
| start/time: | | |

### Dose Response Curve Generation

Test compounds are received in 96 Matrix rack in powder and dissolved in 100% DMSO at 10 mM concentration. In JUMP dilution assay test compounds are tested at a concentration corresponding to 10-fold the IC₅₀ value. The compound IC₅₀ value is previously determined in the primary kinase assay.

Depending on the respective IC₅₀ value, 50-fold concentrated stock solutions in 100% DMSO are prepared from the 10 mM original stock in order to have, for each compound in JUMP mix, a final DMSO concentration of 2%.

Data Analysis

For data quality and data analysis Genedata Screener® 17.0 was used. In the implemented workflow, the data was calculated as follows.
Apply well assignment for controls and test wells.
In genedata Syntax the following well definitions were applied:
   Neutral Control: Central Reference equivalent to Min Signal control
   Stimulator Control: Scale Reference equivalent to Max Signal control
Import of PHERAstar files (txt files containing 665 nm and 620 nm measurements) and FLIPR^{TETRA} files into Genedata Screener software.
Calculate the Response Value (KRV) from the respective original data as follow:

HTRF Assay: Ratio=665 nm_FRET/620 nm_Ab*10000
Calculate the % Activity of the compound (enzymatic recovery effect). KRV was normalized versus Neutral Controls and Stimulator Controls to obtain Activity % according to the following formula:

Activity %=−100*(x−<Neutral Control>)/(<Stimulator Control>−<Neutral Control>)

Where < > indicate KRV median values by plates of the selected control types.

TABLE 2
Additional non-limiting heteroaryl sulfonyl compounds of the present invention and
biological data

| | | CDK2 |
|---|---|---|
| Cmpd | | Kinase |
| # | Structure | (μM) |
| 225 | 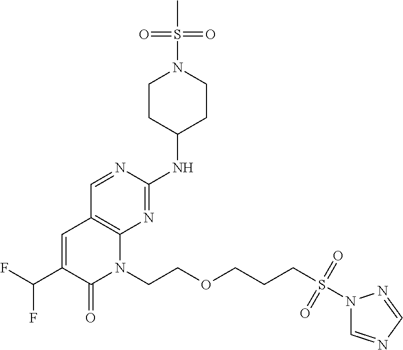 [SMILES: CS(=O)(=O)N1CCC(Nc2ncc3cc(C(F)F)c(=O)n(CCOCCCS(=O)(=O)n4cncn4)c3n2)CC1] | |
| 226 | 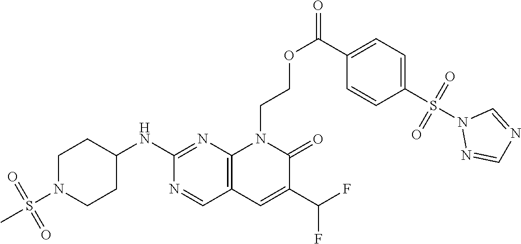 [SMILES: CS(=O)(=O)N1CCC(Nc2ncc3cc(C(F)F)c(=O)n(CCOC(=O)c4ccc(S(=O)(=O)n5cncn5)cc4)c3n2)CC1] | **** |
| 227 | 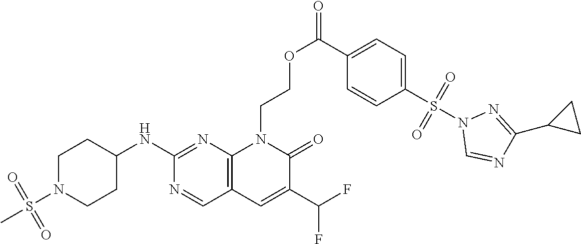 [SMILES: CS(=O)(=O)N1CCC(Nc2ncc3cc(C(F)F)c(=O)n(CCOC(=O)c4ccc(S(=O)(=O)n5cnc(C6CC6)n5)cc4)c3n2)CC1] | **** |
| 228 | 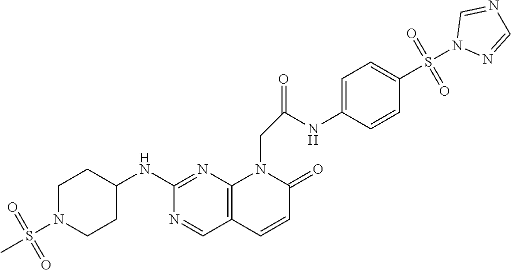 [SMILES: CS(=O)(=O)N1CCC(Nc2ncc3ccc(=O)n(CC(=O)Nc4ccc(S(=O)(=O)n5cncn5)cc4)c3n2)CC1] | **** |
| 229 | 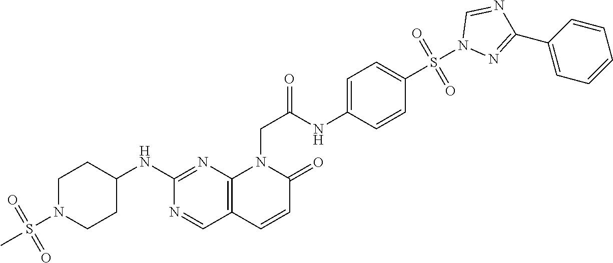 [SMILES: CS(=O)(=O)N1CCC(Nc2ncc3ccc(=O)n(CC(=O)Nc4ccc(S(=O)(=O)n5cnc(-c6ccccc6)n5)cc4)c3n2)CC1] | **** |
| 230 | 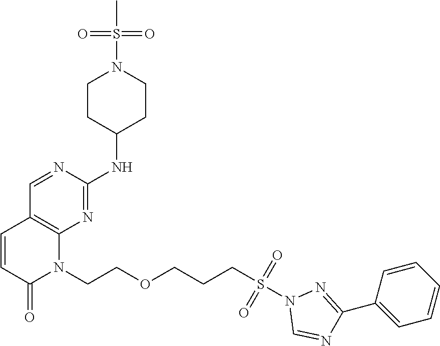 [SMILES: CS(=O)(=O)N1CCC(Nc2ncc3ccc(=O)n(CCOCCCS(=O)(=O)n4cnc(-c5ccccc5)n4)c3n2)CC1] | *** |
| 231 | 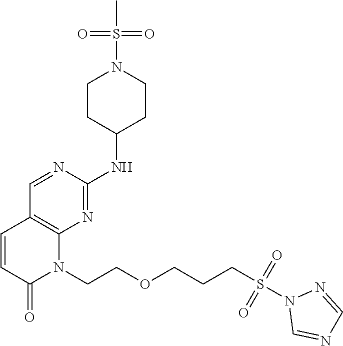 [SMILES: CS(=O)(=O)N1CCC(Nc2ncc3ccc(=O)n(CCOCCCS(=O)(=O)n4cncn4)c3n2)CC1] | **** |
| 232 | 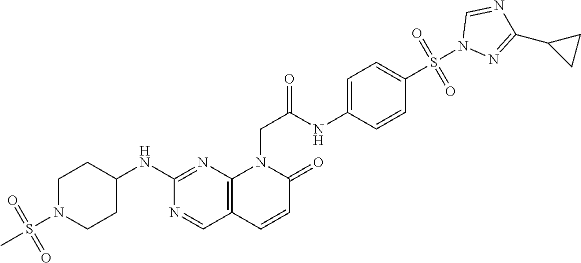 [SMILES: CS(=O)(=O)N1CCC(Nc2ncc3ccc(=O)n(CC(=O)Nc4ccc(S(=O)(=O)n5cnc(C6CC6)n5)cc4)c3n2)CC1] | **** |
| 233 | 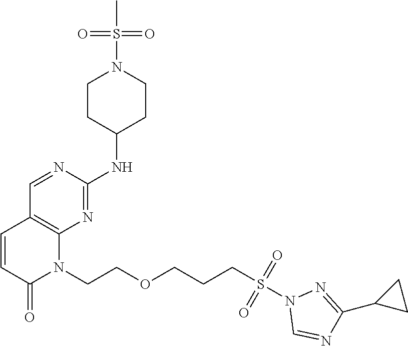 [SMILES: CS(=O)(=O)N1CCC(Nc2ncc3ccc(=O)n(CCOCCCS(=O)(=O)n4cnc(C5CC5)n4)c3n2)CC1] | **** |
| 234 | 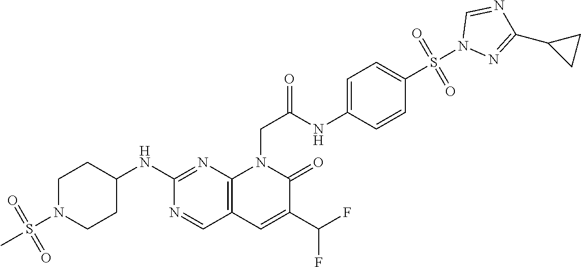 [SMILES: CS(=O)(=O)N1CCC(Nc2ncc3cc(C(F)F)c(=O)n(CC(=O)Nc4ccc(S(=O)(=O)n5cnc(C6CC6)n5)cc4)c3n2)CC1] | **** |
| 235 | 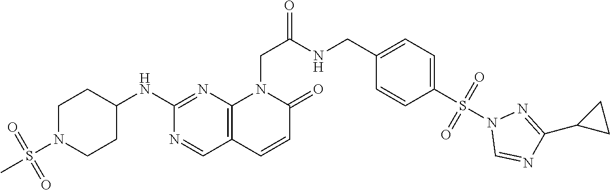 [SMILES: CS(=O)(=O)N1CCC(Nc2ncc3ccc(=O)n(CC(=O)NCc4ccc(S(=O)(=O)n5cnc(C6CC6)n5)cc4)c3n2)CC1] | **** |
| 236 | 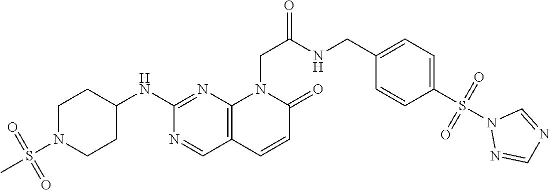 [SMILES: CS(=O)(=O)N1CCC(Nc2ncc3ccc(=O)n(CC(=O)NCc4ccc(S(=O)(=O)n5cncn5)cc4)c3n2)CC1] | **** |
| 237 | 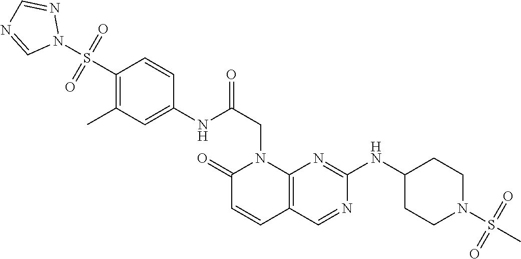 [SMILES: Cc1cc(NC(=O)Cn2c(=O)ccc3cnc(NC4CCN(S(C)(=O)=O)CC4)nc32)ccc1S(=O)(=O)n1cncn1] | **** |
| 238 | 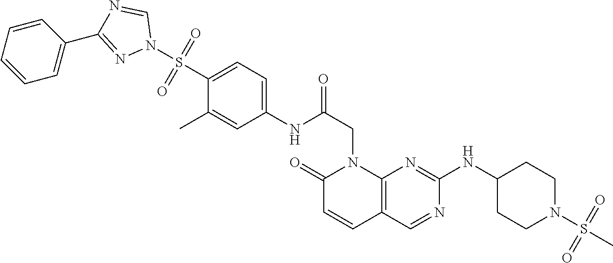 [SMILES: Cc1cc(NC(=O)Cn2c(=O)ccc3cnc(NC4CCN(S(C)(=O)=O)CC4)nc32)ccc1S(=O)(=O)n1cnc(-c2ccccc2)n1] | **** |
| 239 | 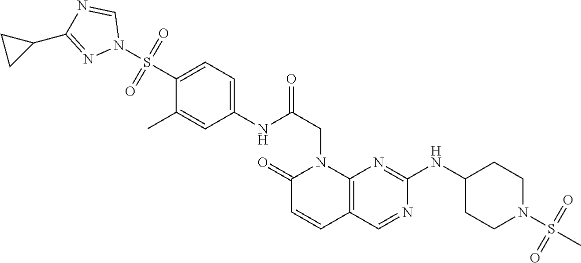 [SMILES: Cc1cc(NC(=O)Cn2c(=O)ccc3cnc(NC4CCN(S(C)(=O)=O)CC4)nc32)ccc1S(=O)(=O)n1cnc(C2CC2)n1] | **** |
| 240 | 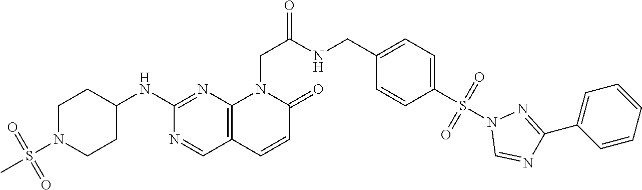 [SMILES: CS(=O)(=O)N1CCC(Nc2ncc3ccc(=O)n(CC(=O)NCc4ccc(S(=O)(=O)n5cnc(-c6ccccc6)n5)cc4)c3n2)CC1] | **** |
| 241 | 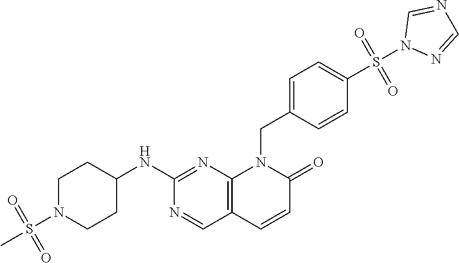 [SMILES: CS(=O)(=O)N1CCC(Nc2ncc3ccc(=O)n(Cc4ccc(S(=O)(=O)n5cncn5)cc4)c3n2)CC1] | **** |
| 242 | 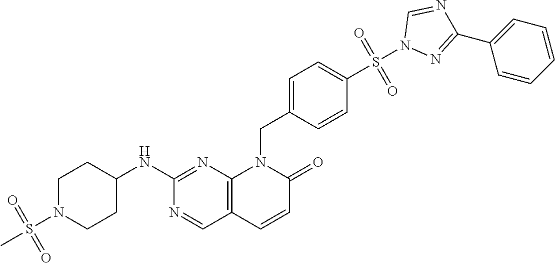 [SMILES: CS(=O)(=O)N1CCC(Nc2ncc3ccc(=O)n(Cc4ccc(S(=O)(=O)n5cnc(-c6ccccc6)n5)cc4)c3n2)CC1] | **** |
| 243 | 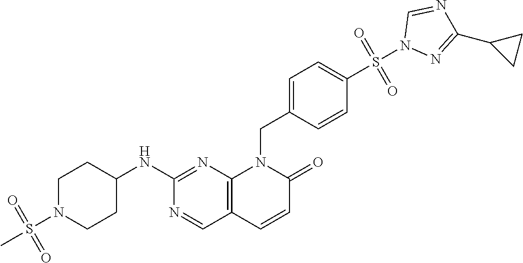 [SMILES: CS(=O)(=O)N1CCC(Nc2ncc3ccc(=O)n(Cc4ccc(S(=O)(=O)n5cnc(C6CC6)n5)cc4)c3n2)CC1] | **** |
| 244 | 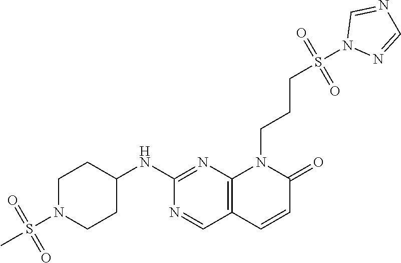 [SMILES: CS(=O)(=O)N1CCC(Nc2ncc3ccc(=O)n(CCCS(=O)(=O)n4cncn4)c3n2)CC1] | **** |
| 245 | 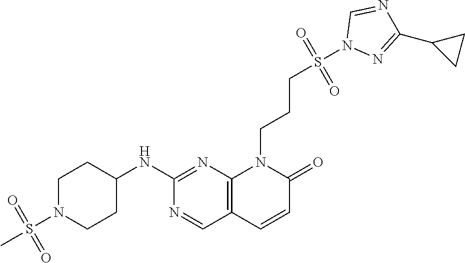 [SMILES: CS(=O)(=O)N1CCC(Nc2ncc3ccc(=O)n(CCCS(=O)(=O)n4cnc(C5CC5)n4)c3n2)CC1] | **** |
| 246 | 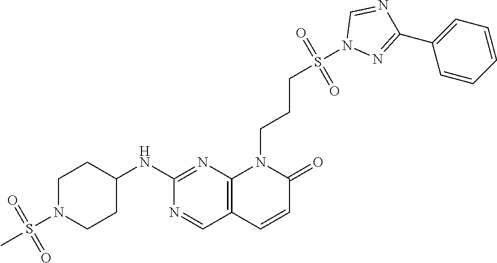 [SMILES: CS(=O)(=O)N1CCC(Nc2ncc3ccc(=O)n(CCCS(=O)(=O)n4cnc(-c5ccccc5)n4)c3n2)CC1] | **** |
| 247 | 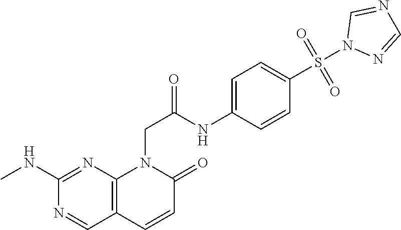 [SMILES: CNc1ncc2ccc(=O)n(CC(=O)Nc3ccc(S(=O)(=O)n4cncn4)cc3)c2n1] | **** |
| 248 | 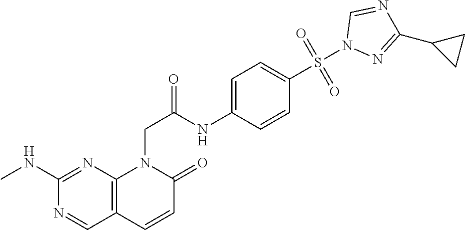 [SMILES: CNc1ncc2ccc(=O)n(CC(=O)Nc3ccc(S(=O)(=O)n4cnc(C5CC5)n4)cc3)c2n1] | **** |
| 249 | 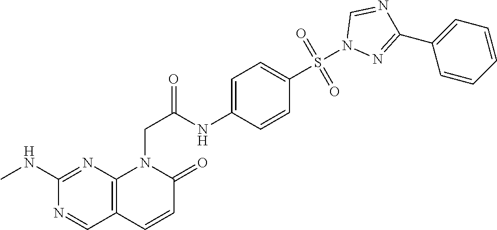 [SMILES: CNc1ncc2ccc(=O)n(CC(=O)Nc3ccc(S(=O)(=O)n4cnc(-c5ccccc5)n4)cc3)c2n1] | * |
| 250 | 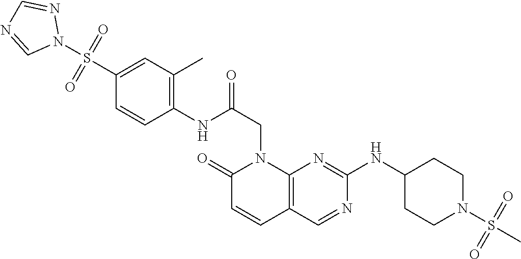 [SMILES: Cc1cc(S(=O)(=O)n2cncn2)ccc1NC(=O)Cn1c(=O)ccc2cnc(NC3CCN(S(C)(=O)=O)CC3)nc21] | **** |
| 251 | 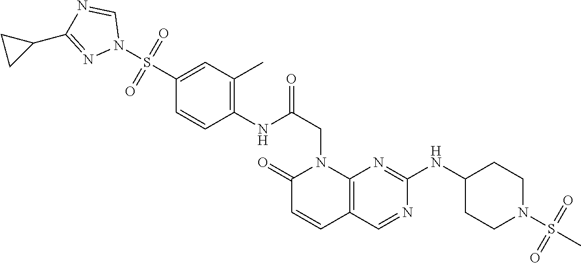 [SMILES: Cc1cc(S(=O)(=O)n2cnc(C3CC3)n2)ccc1NC(=O)Cn1c(=O)ccc2cnc(NC3CCN(S(C)(=O)=O)CC3)nc21] | **** |
| 252 | 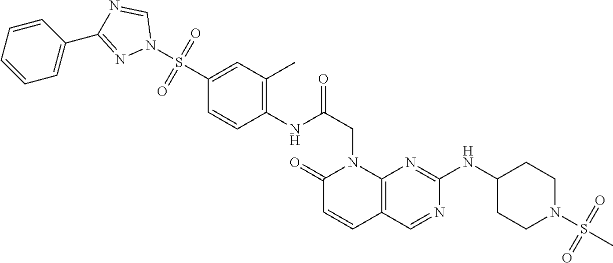 [SMILES: Cc1cc(S(=O)(=O)n2cnc(-c3ccccc3)n2)ccc1NC(=O)Cn1c(=O)ccc2cnc(NC3CCN(S(C)(=O)=O)CC3)nc21] | **** |
| 253 | 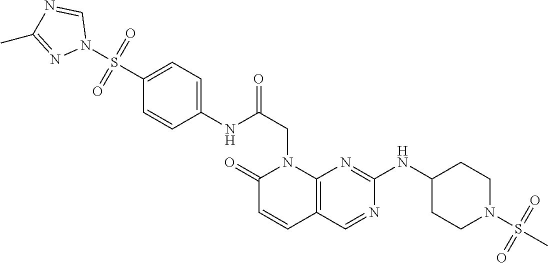 | **** |
| 254 | 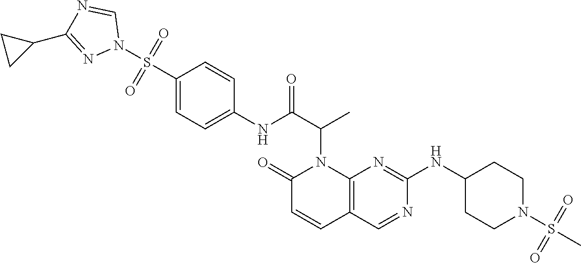 [SMILES: CC(C(=O)Nc1ccc(S(=O)(=O)n2cnc(C3CC3)n2)cc1)n1c(=O)ccc2cnc(NC3CCN(S(C)(=O)=O)CC3)nc21] | **** |
| 255 | 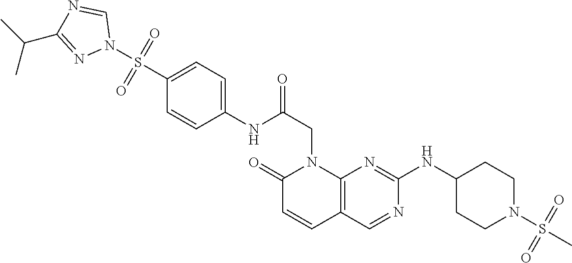 [SMILES: CC(C)c1ncn(S(=O)(=O)c2ccc(NC(=O)Cn3c(=O)ccc4cnc(NC5CCN(S(C)(=O)=O)CC5)nc43)cc2)n1] | **** |
| 256 | 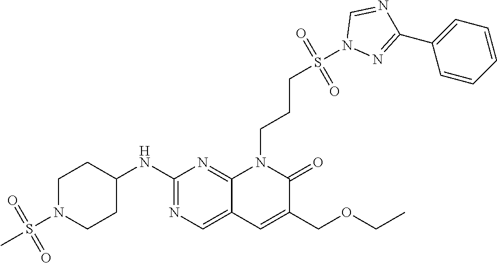 [SMILES: CCOCc1cc2cnc(NC3CCN(S(C)(=O)=O)CC3)nc2n(CCCS(=O)(=O)n2cnc(-c3ccccc3)n2)c1=O] | * |
| 257 | 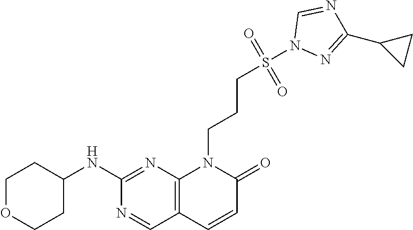 [SMILES: O=c1ccc2cnc(NC3CCOCC3)nc2n1CCCS(=O)(=O)n1cnc(C2CC2)n1] | **** |
| 258 | 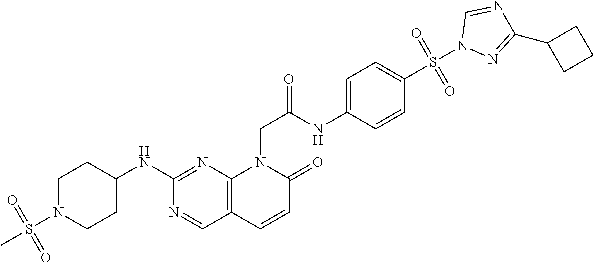 [SMILES: CS(=O)(=O)N1CCC(Nc2ncc3ccc(=O)n(CC(=O)Nc4ccc(S(=O)(=O)n5cnc(C6CCC6)n5)cc4)c3n2)CC1] | * |
| 259 | 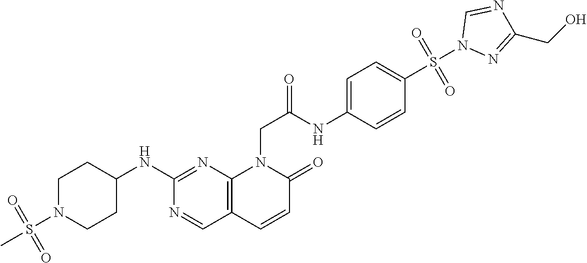 [SMILES: CS(=O)(=O)N1CCC(Nc2ncc3ccc(=O)n(CC(=O)Nc4ccc(S(=O)(=O)n5cnc(CO)n5)cc4)c3n2)CC1] | **** |
| 260 | 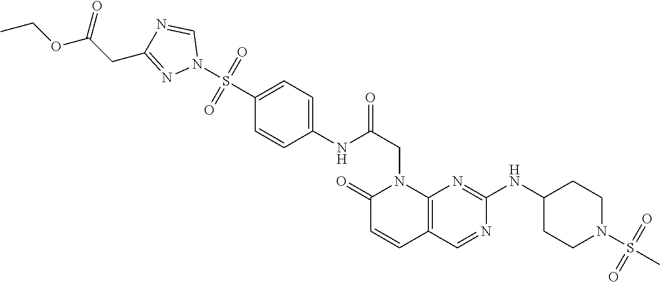 [SMILES: CCOC(=O)Cc1ncn(S(=O)(=O)c2ccc(NC(=O)Cn3c(=O)ccc4cnc(NC5CCN(S(C)(=O)=O)CC5)nc43)cc2)n1] | **** |
| 261 | 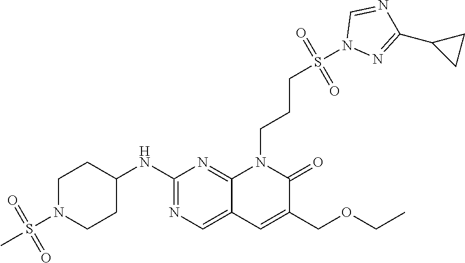 [SMILES: CCOCc1cc2cnc(NC3CCN(S(C)(=O)=O)CC3)nc2n(CCCS(=O)(=O)n2cnc(C3CC3)n2)c1=O] | *** |
| 262 | 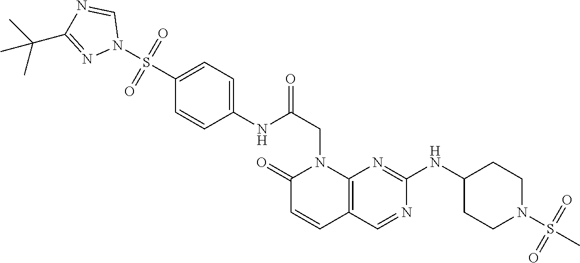 [SMILES: CC(C)(C)c1ncn(S(=O)(=O)c2ccc(NC(=O)Cn3c(=O)ccc4cnc(NC5CCN(S(C)(=O)=O)CC5)nc43)cc2)n1] | * |
| 263 | 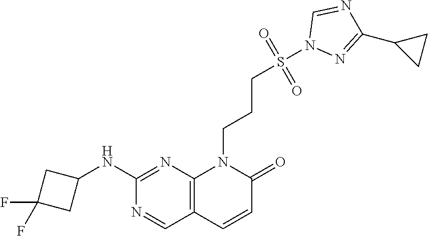 [SMILES: O=c1ccc2cnc(NC3CC(F)(F)C3)nc2n1CCCS(=O)(=O)n1cnc(C2CC2)n1] | **** |
| 264 | 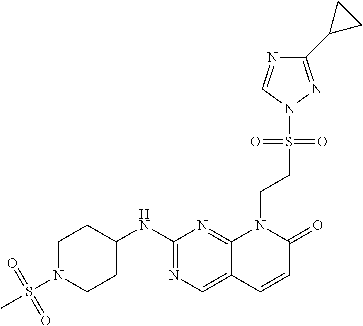 [SMILES: CS(=O)(=O)N1CCC(Nc2ncc3ccc(=O)n(CCS(=O)(=O)n4cnc(C5CC5)n4)c3n2)CC1] | **** |
| 265 | 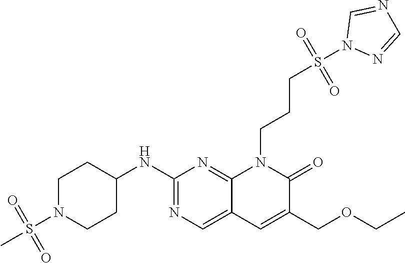 [SMILES: CCOCc1cc2cnc(NC3CCN(S(C)(=O)=O)CC3)nc2n(CCCS(=O)(=O)n2cncn2)c1=O] | *** |
| 266 | 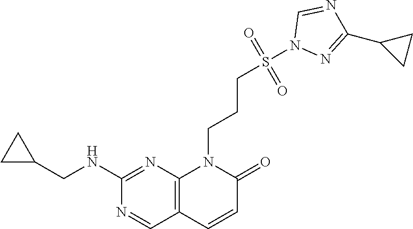 [SMILES: O=c1ccc2cnc(NCC3CC3)nc2n1CCCS(=O)(=O)n1cnc(C2CC2)n1] | *** |
| 267 | 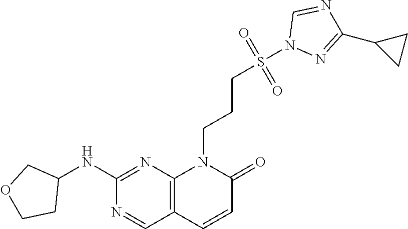 [SMILES: O=c1ccc2cnc(NC3CCOC3)nc2n1CCCS(=O)(=O)n1cnc(C2CC2)n1] | *** |
| 268 | 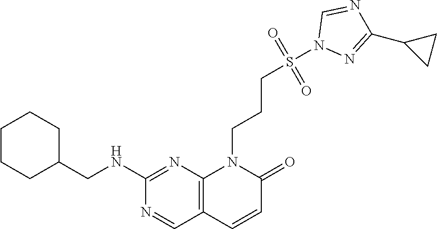 [SMILES: O=c1ccc2cnc(NCC3CCCCC3)nc2n1CCCS(=O)(=O)n1cnc(C2CC2)n1] | * |
| 269 | 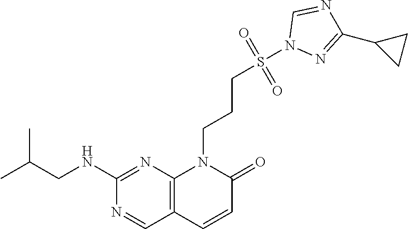 [SMILES: CC(C)CNc1ncc2ccc(=O)n(CCCS(=O)(=O)n3cnc(C4CC4)n3)c2n1] | **** |
| 270 | 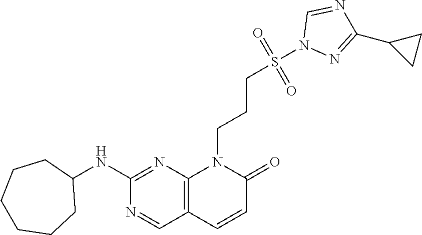 [SMILES: O=c1ccc2cnc(NC3CCCCCC3)nc2n1CCCS(=O)(=O)n1cnc(C2CC2)n1] | **** |
| 271 | 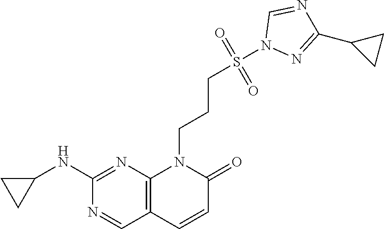 [SMILES: O=c1ccc2cnc(NC3CC3)nc2n1CCCS(=O)(=O)n1cnc(C2CC2)n1] | **** |
| 272 | 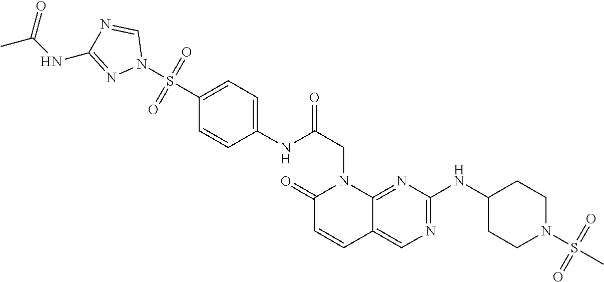 [SMILES: CC(=O)Nc1ncn(S(=O)(=O)c2ccc(NC(=O)Cn3c(=O)ccc4cnc(NC5CCN(S(C)(=O)=O)CC5)nc43)cc2)n1] | **** |
| 273 | 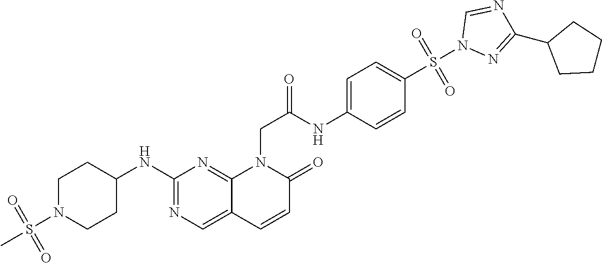 [SMILES: CS(=O)(=O)N1CCC(Nc2ncc3ccc(=O)n(CC(=O)Nc4ccc(S(=O)(=O)n5cnc(C6CCCC6)n5)cc4)c3n2)CC1] | * |
| 274 | 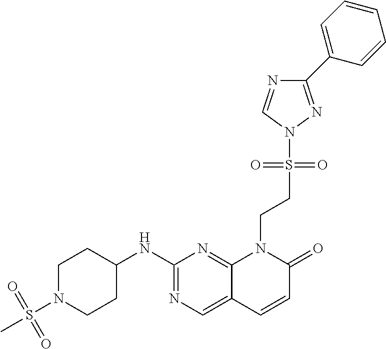 [SMILES: CS(=O)(=O)N1CCC(Nc2ncc3ccc(=O)n(CCS(=O)(=O)n4cnc(-c5ccccc5)n4)c3n2)CC1] | **** |
| 275 | 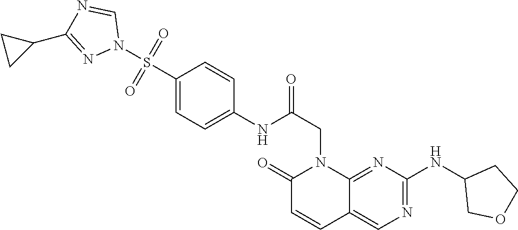 [SMILES: O=C(Cn1c(=O)ccc2cnc(NC3CCOC3)nc21)Nc1ccc(S(=O)(=O)n2cnc(C3CC3)n2)cc1] | **** |
| 276 | 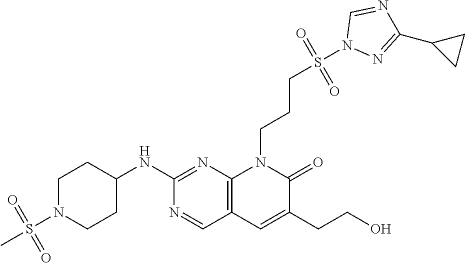 [SMILES: CS(=O)(=O)N1CCC(Nc2ncc3cc(CCO)c(=O)n(CCCS(=O)(=O)n4cnc(C5CC5)n4)c3n2)CC1] | **** |
| 277 | 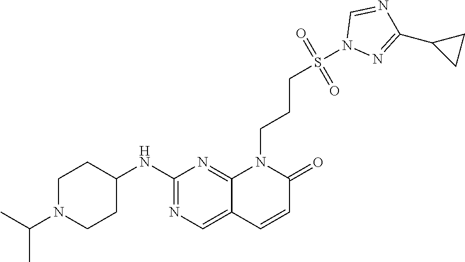 [SMILES: CC(C)N1CCC(Nc2ncc3ccc(=O)n(CCCS(=O)(=O)n4cnc(C5CC5)n4)c3n2)CC1] | * |
| 278 | 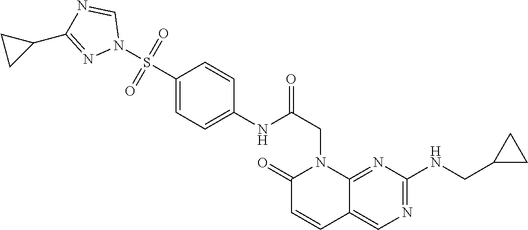 [SMILES: O=C(Cn1c(=O)ccc2cnc(NCC3CC3)nc21)Nc1ccc(S(=O)(=O)n2cnc(C3CC3)n2)cc1] | * |
| 279 | 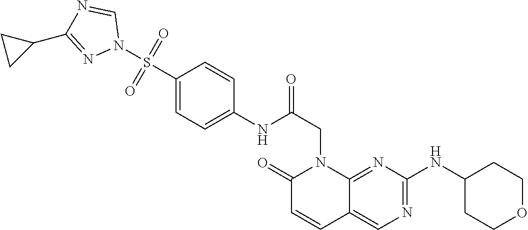 [SMILES: O=C(Cn1c(=O)ccc2cnc(NC3CCOCC3)nc21)Nc1ccc(S(=O)(=O)n2cnc(C3CC3)n2)cc1] | **** |
| 280 | 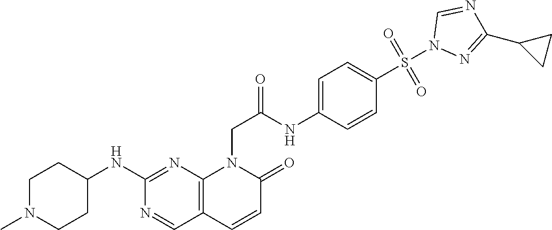 [SMILES: CN1CCC(Nc2ncc3ccc(=O)n(CC(=O)Nc4ccc(S(=O)(=O)n5cnc(C6CC6)n5)cc4)c3n2)CC1] | * |
| 281 | 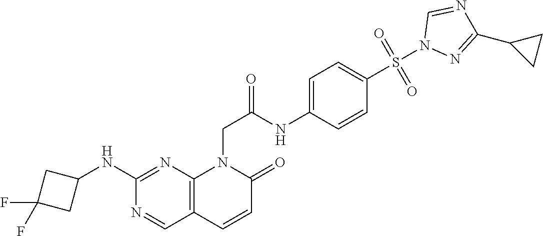 [SMILES: O=C(Cn1c(=O)ccc2cnc(NC3CC(F)(F)C3)nc21)Nc1ccc(S(=O)(=O)n2cnc(C3CC3)n2)cc1] | **** |
| 282 | 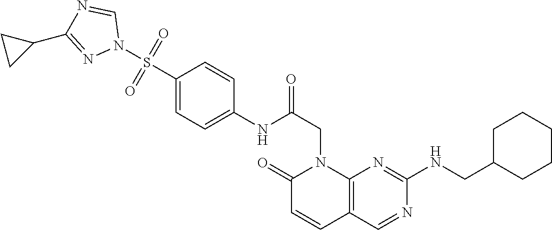 [SMILES: O=C(Cn1c(=O)ccc2cnc(NCC3CCCCC3)nc21)Nc1ccc(S(=O)(=O)n2cnc(C3CC3)n2)cc1] | * |
| 283 | 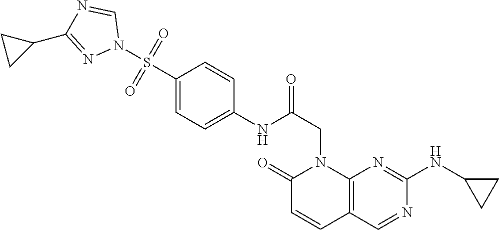 [SMILES: O=C(Cn1c(=O)ccc2cnc(NC3CC3)nc21)Nc1ccc(S(=O)(=O)n2cnc(C3CC3)n2)cc1] | **** |
| 284 | 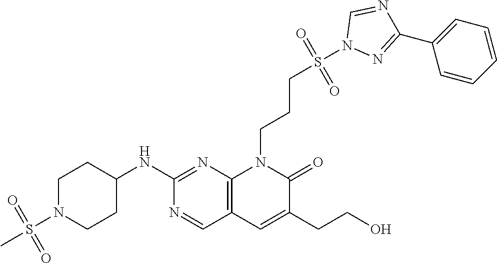 [SMILES: CS(=O)(=O)N1CCC(Nc2ncc3cc(CCO)c(=O)n(CCCS(=O)(=O)n4cnc(-c5ccccc5)n4)c3n2)CC1] | **** |
| 285 | 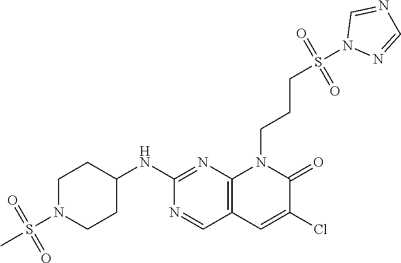 [SMILES: CS(=O)(=O)N1CCC(Nc2ncc3cc(Cl)c(=O)n(CCCS(=O)(=O)n4cncn4)c3n2)CC1] | **** |
| 286 | 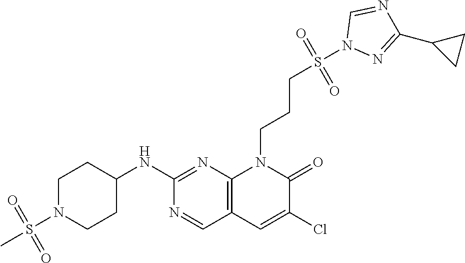 [SMILES: CS(=O)(=O)N1CCC(Nc2ncc3cc(Cl)c(=O)n(CCCS(=O)(=O)n4cnc(C5CC5)n4)c3n2)CC1] | **** |
| 287 | 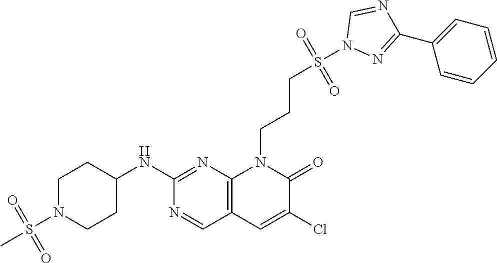 [SMILES: CS(=O)(=O)N1CCC(Nc2ncc3cc(Cl)c(=O)n(CCCS(=O)(=O)n4cnc(-c5ccccc5)n4)c3n2)CC1] | **** |
| 288 | 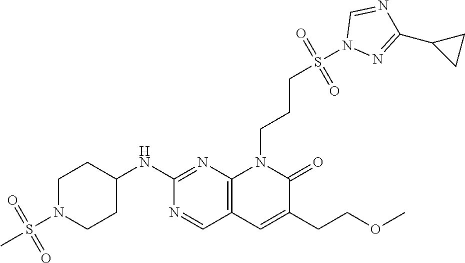 [SMILES: COCCc1cc2cnc(NC3CCN(S(C)(=O)=O)CC3)nc2n(CCCS(=O)(=O)n2cnc(C3CC3)n2)c1=O] | **** |
| 289 | 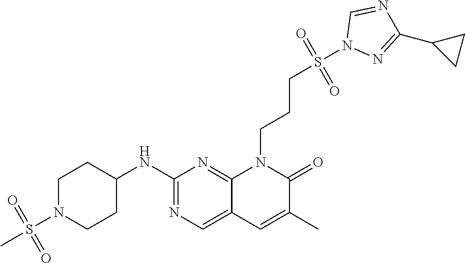 [SMILES: Cc1cc2cnc(NC3CCN(S(C)(=O)=O)CC3)nc2n(CCCS(=O)(=O)n2cnc(C3CC3)n2)c1=O] | **** |
| 290 | 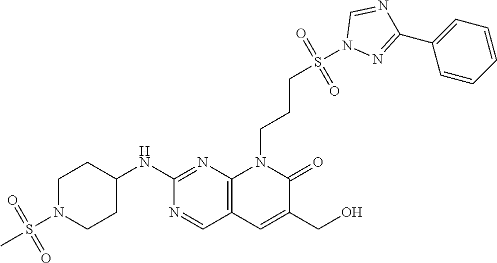 [SMILES: CS(=O)(=O)N1CCC(Nc2ncc3cc(CO)c(=O)n(CCCS(=O)(=O)n4cnc(-c5ccccc5)n4)c3n2)CC1] | **** |
| 291 | 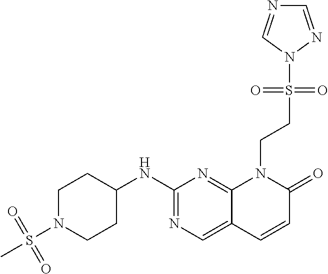 [SMILES: CS(=O)(=O)N1CCC(Nc2ncc3ccc(=O)n(CCS(=O)(=O)n4cncn4)c3n2)CC1] | **** |
| 292 | 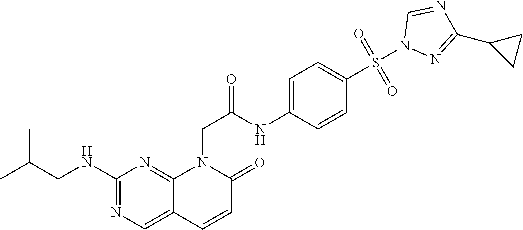 [SMILES: CC(C)CNc1ncc2ccc(=O)n(CC(=O)Nc3ccc(S(=O)(=O)n4cnc(C5CC5)n4)cc3)c2n1] | * |
| 293 | 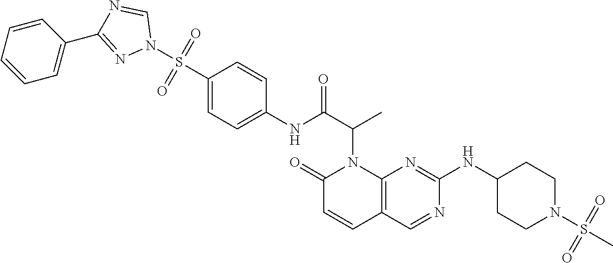 [SMILES: CC(C(=O)Nc1ccc(S(=O)(=O)n2cnc(-c3ccccc3)n2)cc1)n1c(=O)ccc2cnc(NC3CCN(S(C)(=O)=O)CC3)nc21] | **** |
| 294 | 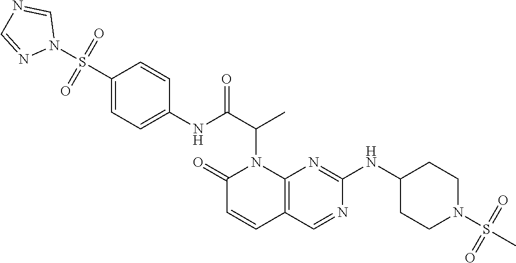 [SMILES: CC(C(=O)Nc1ccc(S(=O)(=O)n2cncn2)cc1)n1c(=O)ccc2cnc(NC3CCN(S(C)(=O)=O)CC3)nc21] | **** |
| 295 | 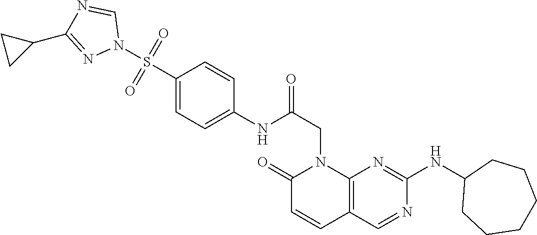 [SMILES: O=C(Cn1c(=O)ccc2cnc(NC3CCCCCC3)nc21)Nc1ccc(S(=O)(=O)n2cnc(C3CC3)n2)cc1] | * |
| 296 | 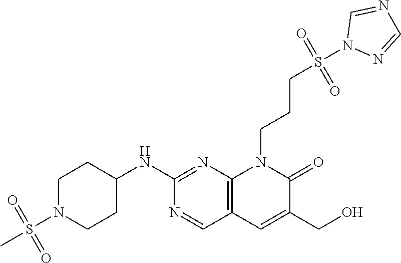 [SMILES: CS(=O)(=O)N1CCC(Nc2ncc3cc(CO)c(=O)n(CCCS(=O)(=O)n4cncn4)c3n2)CC1] | **** |
| 297 | 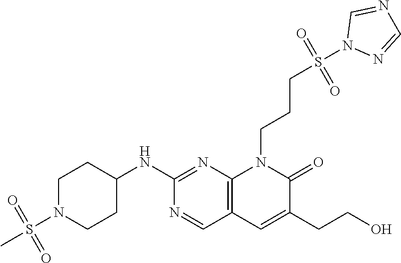 [SMILES: CS(=O)(=O)N1CCC(Nc2ncc3cc(CCO)c(=O)n(CCCS(=O)(=O)n4cncn4)c3n2)CC1] | **** |
| 298 | 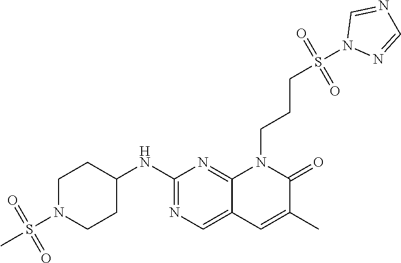 [SMILES: Cc1cc2cnc(NC3CCN(S(C)(=O)=O)CC3)nc2n(CCCS(=O)(=O)n2cncn2)c1=O] | **** |
| 299 | 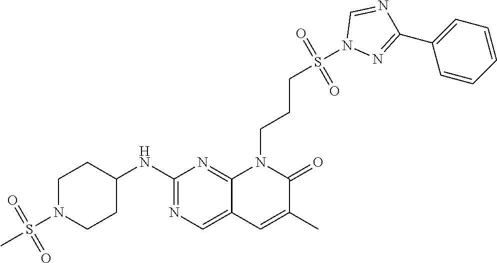 [SMILES: Cc1cc2cnc(NC3CCN(S(C)(=O)=O)CC3)nc2n(CCCS(=O)(=O)n2cnc(-c3ccccc3)n2)c1=O] | **** |
| 300 | 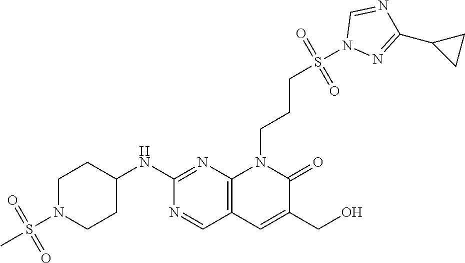 [SMILES: CS(=O)(=O)N1CCC(Nc2ncc3cc(CO)c(=O)n(CCCS(=O)(=O)n4cnc(C5CC5)n4)c3n2)CC1] | **** |

**** is < = 10 μM, *** is < = 100 μM, ** is < = 200 μM, * is >200 μM

All publications and patent applications cited in this specification are herein incorporated by reference as if each individual publication or patent application were specifically and individually indicated to be incorporated by reference.

Although the foregoing invention has been described in some detail by way of illustration and example for the purposes of clarity of understanding, it will be readily apparent to one of ordinary skill in the art in light of the teaching of this invention that certain changes and modification may be made thereto without departing from the spirit or scope of the invention as defined in the claims.

We claim:

1. A pharmaceutically acceptable compound of Formula: 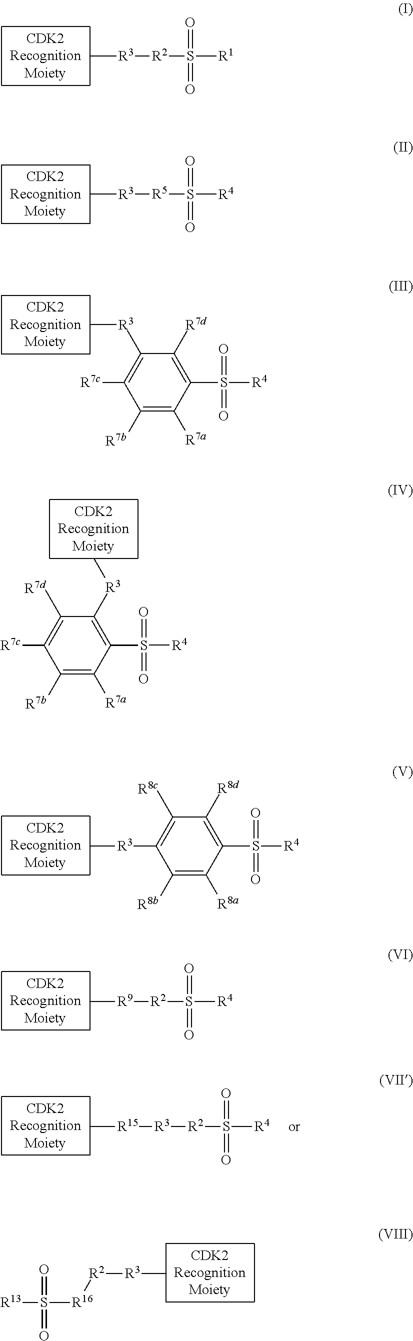
or a pharmaceutically acceptable salt thereof,
wherein:
R¹ is selected from the group consisting of:
a) 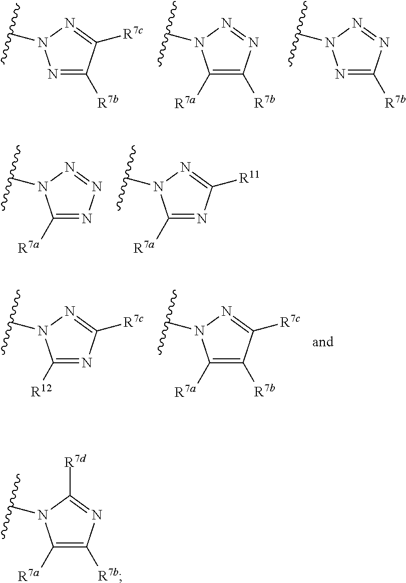
or
b) a bicyclic heteroaryl which is optionally substituted with 1, 2, or 3 substituents selected from R⁷;
R² is independently selected at each instance from the group consisting of bond, alkyl, cycloalkyl, aryl, heterocycle, —S—, —O—, —NR⁶—, —(CH₂)ₚ—C(O)—NR⁶—, —(CH₂CH₂O)ₚ—, —(OCH₂CH₂)ₚ—, —NR⁶C(O)NR⁶—, —C(O)NR⁶—, —OC(O)NR⁶—, aryl-C(O)—NR⁶—, heteroaryl-C(O)—NR⁶—, bicycle, and heteroaryl, each of which is optionally substituted with 1, 2, or 3 substituents selected from R⁷;
R³ is independently selected at each instance from bond, alkyl, cycloalkyl, aryl, heterocycle, —S—, —O—, —NR⁶—, —(CH₂)ₚ—C(O)—, —(CH₂)ₚ—C(O)—NR⁶—, —(CH₂CH₂O)ₚ—, —(OCH₂CH₂)ₚ—, —C(O)—, —NR⁶C(O)—, —NR⁶C(O)NR⁶—, —C(O)NR⁶—, —OC(O)NR⁶—, —NR⁶S(O)₂NR⁶—, —S(O)₂NR⁶—, aryl-C(O)—NR⁶—, heteroaryl-C(O)—NR⁶—, bicycle, and heteroaryl, each of which is optionally substituted with 1, 2, or 3 substituents selected from R⁷;
p is independently selected from 1, 2, 3, 4, 5, and 6;
R⁴ is a heteroaryl group with 1, 2, 3, or 4 nitrogen atoms, where the bond to the sulfur atom is through one of the nitrogen atoms present in the cycle, and each heteroaryl is optionally substituted with 1, 2, or 3 substituents selected from R⁷;
R⁵ is selected from the group consisting of alkyl, cycloalkyl, naphthyl, heterocycle, —S—, —O—, —NR⁶—, —(CH₂)ₚ—C(O)—NR⁶—, —(CH₂CH₂O)ₚ—, —(OCH₂CH₂)ₚ—, —NR⁶C(O)NR⁶—, —C(O)NR⁶—, —OC(O)NR⁶—, heteroaryl-C(O)—NR⁶—, bicycle, and heteroaryl, each of which is optionally substituted with 1, 2, or 3 substituents selected from R⁷;
R⁶ is independently selected at each instance hydrogen or alkyl;
R⁷, R^{7a}, R^{7b}, R^{7c}, and R^{7d} are independently selected at each instance from the group consisting of hydrogen, halogen, alkyl, haloalkyl, alkenyl, cycloalkyl, heterocycle, aryl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, —S(O)R⁶, —S(O)₂R⁶, —S(O)OR⁶, —S(O)₂OR⁶, —S(O)N(R⁶)₂, S(O)₂N(R⁶)₂, and —SR⁶, wherein each alkyl, haloalkyl, alkenyl, cycloalkyl, heterocycle, aryl, and heteroaryl is optionally substituted with 1, 2, or 3 substituents selected from R¹⁷;
R¹⁷ is independently selected in each instance from the group consisting of hydrogen, halogen, alkyl, haloalkyl, alkenyl, cycloalkyl, heterocycle, aryl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, —S(O)R⁶, —S(O)₂R⁶, —S(O)OR⁶, —S(O)₂OR⁶, —S(O)N(R⁶)₂, —S(O)₂N(R⁶)₂, and —SR⁶;
R^{8a}, R^{8b}, R^{8c}, and R^{8d} are independently selected at each instance from the group consisting of R⁷ and R¹² wherein at least one of R^{8a}, R^{8b}, R^{8c}, and R^{8d} is R¹²;
R⁹ is alkyl, alkenyl, haloalkyl, cycloalkyl, heterocycle, —NR⁶C(O)—, —NR⁶C(O)NR⁶—, —C(O)NR⁶—, —OC(O)NR⁶—, —NR⁶S(O)₂NR⁶—, —S(O)₂NR⁶—, bicycle, or heteroaryl, each of which is optionally substituted with 1, 2, or 3 substituents selected from R⁷;
R¹¹ is hydrogen, halogen, alkyl, haloalkyl, alkenyl, cycloalkyl, heterocycle, naphthyl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, or —SR⁶, wherein each alkyl, haloalkyl, alkenyl, cycloalkyl, heterocycle, aryl, and heteroaryl optionally substituted with 1, 2, or 3 substituents selected from R¹⁷;
R¹² is halogen, alkyl, haloalkyl, alkenyl, cycloalkyl, heterocycle, aryl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, —S(O)R⁶, —S(O)₂R⁶, —S(O)OR⁶, —S(O)₂OR⁶, —S(O)N(R⁶)₂, S(O)₂N(R⁶)₂, or —SR⁶, wherein each alkyl, haloalkyl, alkenyl, cycloalkyl, heterocycle, aryl, and heteroaryl is optionally substituted with 1, 2, or 3 substituents selected from R¹⁷;
R¹³ is alkyl, haloalkyl, cycloalkyl, heterocycle, heteroaryl, aryl, —OR⁶, —N(R⁶)₂, —C(O)R⁶, —NR⁶C(O)R⁶, —C(O)N(R⁶)₂, or —NR⁶C(O)N(R⁶)₂, each of which is optionally substituted with 1, 2, or 3 substituents selected from R⁷;
R¹⁵ is alkyl, alkenyl, haloalkyl, cycloalkyl, aryl, heterocycle, —S—, —O—, —NR⁶—, —S-alkyl-, —O-alkyl-, —NR⁶-alkyl-, -alkyl-C(O)—, -alkyl-C(O)-alkyl-, -alkyl-C(O)—NR⁶-alkyl-, —C(O)—NR⁶-alkyl-, -alkyl-C(O)—NR⁶—, -alkyl-C(O)—O-alkyl-, —C(O)—O-alkyl-, -alkyl-C(O)—O—, —C(O)—, —NR⁶C(O)NR⁶—, —C(O)NR⁶—, —OC(O)NR⁶—, —NR⁶S(O)₂NR⁶—, —S(O)₂NR⁶—, bicycle, or heteroaryl, each of which except bond is optionally substituted with 1, 2, or 3 substituents independently selected from R⁷;
R¹⁶ is a heteroaryl group, where the bond to the sulfur atom is through one of the nitrogen atoms present in the cycle, and R¹⁶ is optionally substituted with 1, 2, or 3 substituents selected from R⁷; and
CDK2 Recognition Moiety is a molecule which can bind to or otherwise anchors to CDK2.

2. The pharmaceutically acceptable compound of claim 1, wherein R³ is phenyl, alkyl, or heteroaryl optionally substituted with 1, 2, or 3 substituents selected from R⁷.

3. The pharmaceutically acceptable compound of claim 2, wherein the compound is of Formula: 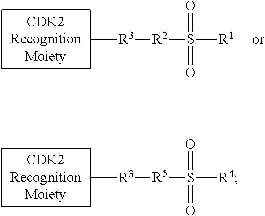
or a pharmaceutically acceptable salt thereof.

4. The pharmaceutically acceptable compound of claim 3, wherein the compound is not substituted.

5. The pharmaceutically acceptable compound of claim 3, wherein R¹ and R⁴ are

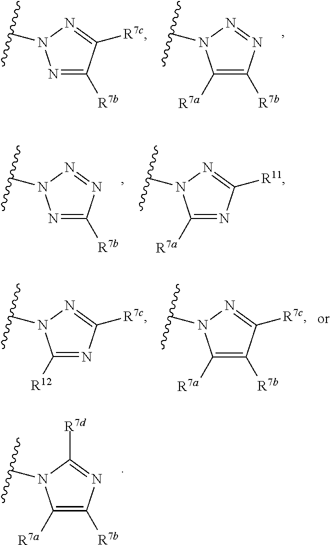

6. The pharmaceutically acceptable compound of claim 5, wherein R² is bond.

7. The pharmaceutically acceptable compound of claim 5, wherein R² is phenyl, alkyl, or heteroaryl optionally substituted with 1, 2, or 3 substituents selected from R⁷.

8. The pharmaceutically acceptable compound of claim 2, wherein the compound is Formula: 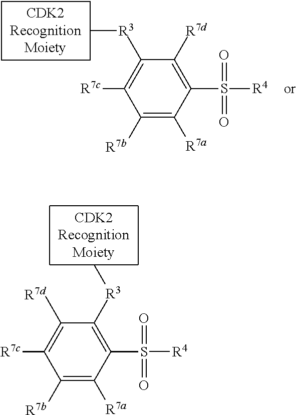
or a pharmaceutically acceptable salt thereof.

9. The pharmaceutically acceptable compound of claim 8, wherein the compound is not substituted.

10. The pharmaceutically acceptable compound of claim 2, wherein the compound is Formula: 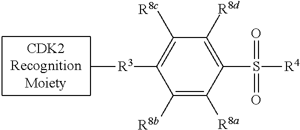
or a pharmaceutically acceptable salt thereof.

11. The pharmaceutically acceptable compound of claim 10, wherein the compound is not substituted.

12. The pharmaceutically acceptable compound of claim 2, wherein the compound is Formula: 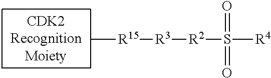
or a pharmaceutically acceptable salt thereof.

13. The pharmaceutically acceptable compound of claim 12, wherein the compound is not substituted.

14. The pharmaceutically acceptable compound of claim 12, wherein R⁴ is

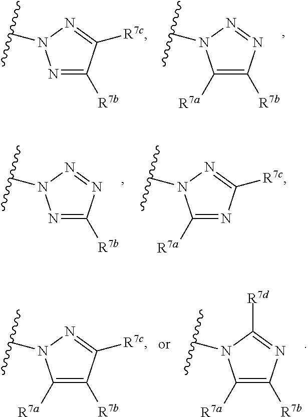

15. The pharmaceutically acceptable compound of claim 14, wherein R² is phenyl, alkyl, or heteroaryl optionally substituted with 1, 2, or 3 substituents selected from R⁷.

16. The pharmaceutically acceptable compound of claim 2, wherein the compound is of Formula: 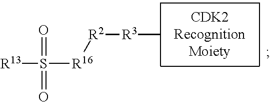
or a pharmaceutically acceptable salt thereof.

17. The pharmaceutically acceptable compound of claim 16, wherein the compound is not substituted.

18. The pharmaceutically acceptable compound of claim 16, wherein R¹³ is phenyl optionally substituted with 1, 2, or 3 substituents selected from R⁷.

19. The pharmaceutically acceptable compound of claim 16, wherein R³ is alkyl optionally substituted with 1, 2, or 3 substituents selected from R⁷.

20. The pharmaceutically acceptable compound of claim 16, wherein R¹⁶ is

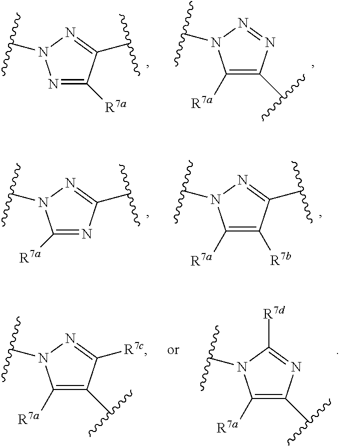

21. The pharmaceutically acceptable compound of claim 2, wherein the compound is of Formula: 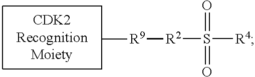
or a pharmaceutically acceptable salt thereof.

22. The pharmaceutically acceptable compound of claim 21, wherein the compound is not substituted.

23. The pharmaceutically acceptable compound of claim 21, wherein R⁴ is

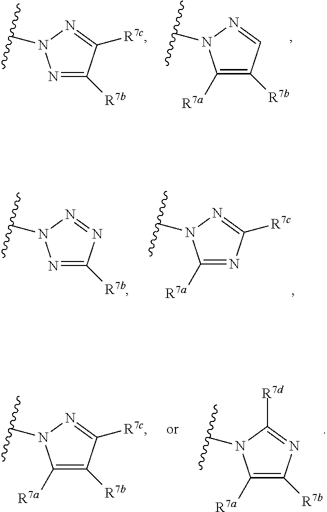

24. The pharmaceutically acceptable compound of claim 23, wherein R⁹ is selected from alkyl, cycloalkyl, and heteroaryl, each of which except bond is optionally substituted with 1, 2, or 3 substituents independently selected from R⁷.

25. The pharmaceutically acceptable compound of claim 2, wherein CDK2 Recognition Moiety is 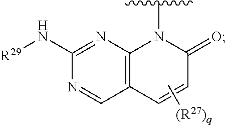
wherein
R²⁷ is independently selected at each instance from the group consisting of hydrogen, halogen, alkyl, haloalkyl, alkenyl, cycloalkyl, heterocycle, aryl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, —S(O)R⁶, —S(O)₂R⁶, —S(O)OR⁶, —S(O)₂OR⁶, —S(O)N(R⁶)₂, S(O)₂N(R⁶)₂, and —SR;
R²⁹ is independently selected at each instance from alkyl, haloalkyl, cycloalkyl, aryl, heterocycle, and heteroaryl each of which is optionally substituted with 1 or 2 substituents independently selected from R¹⁷; and
q is 0, 1, or 2.

26. The pharmaceutically acceptable compound of claim 25, wherein q is 0.

27. The pharmaceutically acceptable compound of claim 26, wherein R²⁹ is cycloalkyl, aryl, heterocycle, or heteroaryl each of which is optionally substituted with 1 or 2 substituents independently selected from R¹⁷.

28. The pharmaceutically acceptable compound of claim 2, wherein CDK2 Recognition Moiety is 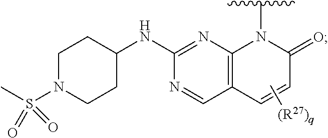
wherein
R²⁷ is independently selected at each instance from the group consisting of hydrogen, halogen, alkyl, haloalkyl, alkenyl, cycloalkyl, heterocycle, aryl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, —S(O)R⁶, —S(O)₂R⁶, —S(O)OR⁶, —S(O)₂OR⁶, —S(O)N(R⁶)₂, S(O)₂N(R⁶)₂, and —SR;
R²⁹ is independently selected at each instance from hydrogen, alkyl, haloalkyl, cycloalkyl, aryl, heterocycle, and heteroaryl each of which is optionally substituted with 1 or 2 substituents independently selected from R¹⁷; and
q is 0, 1, 2, or 3.

29. The pharmaceutically acceptable compound of claim 28, wherein q is 0.

30. The pharmaceutically acceptable compound of claim 1 of structure: 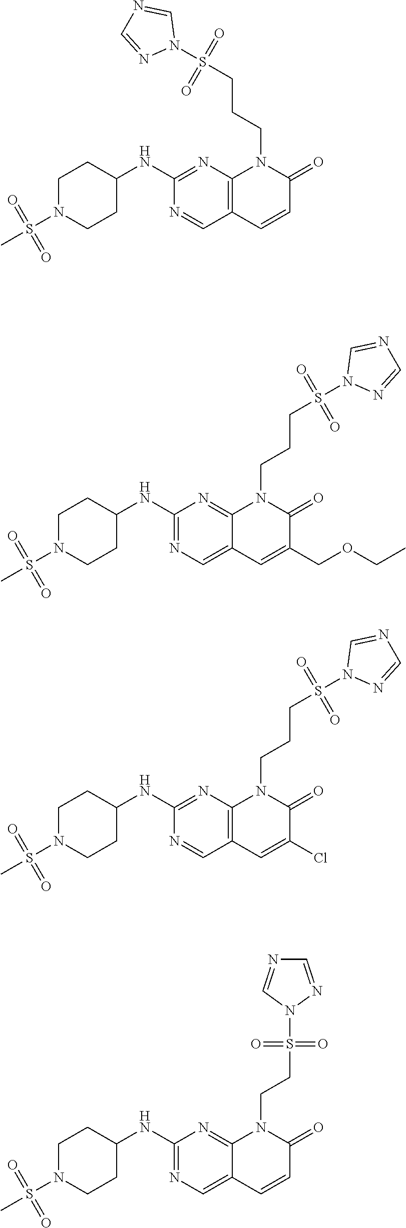 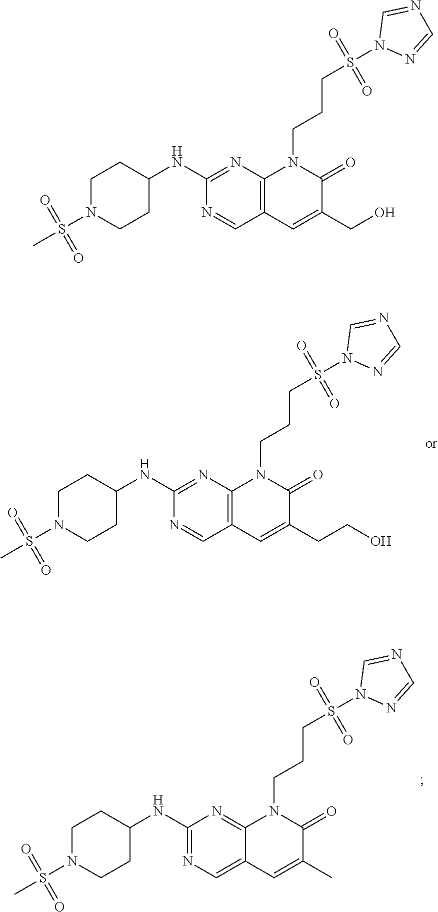 [SMILES: CS(=O)(=O)N1CCC(Nc2ncc3cc(CO)c(=O)n(CCCS(=O)(=O)n4cncn4)c3n2)CC1 ; CS(=O)(=O)N1CCC(Nc2ncc3cc(CCO)c(=O)n(CCCS(=O)(=O)n4cncn4)c3n2)CC1 ; Cc1cc2cnc(NC3CCN(S(C)(=O)=O)CC3)nc2n(CCCS(=O)(=O)n2cncn2)c1=O]
or a pharmaceutically acceptable salt thereof.

31. The pharmaceutically acceptable compound of claim 1 of structure: 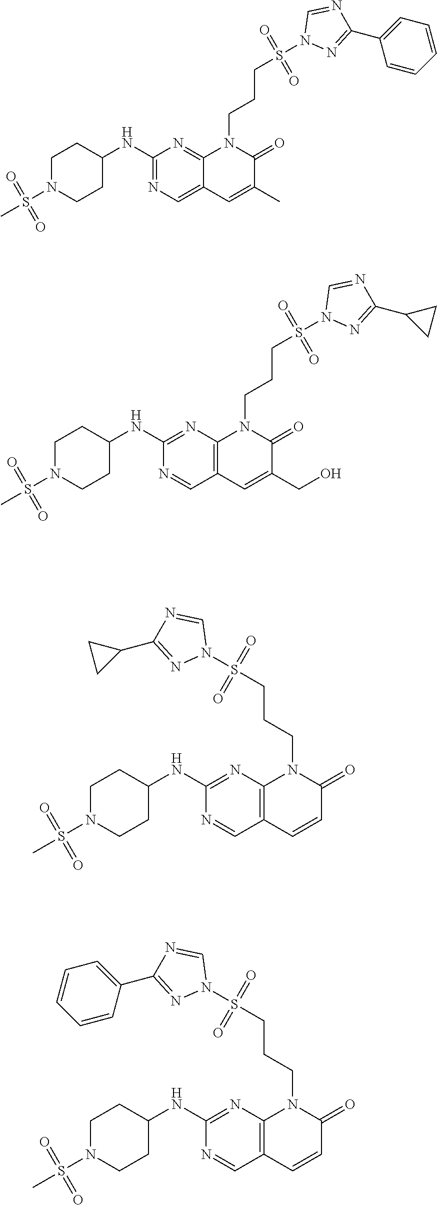 [SMILES: Cc1cc2cnc(NC3CCN(S(C)(=O)=O)CC3)nc2n(CCCS(=O)(=O)n2cnc(-c3ccccc3)n2)c1=O ; CS(=O)(=O)N1CCC(Nc2ncc3cc(CO)c(=O)n(CCCS(=O)(=O)n4cnc(C5CC5)n4)c3n2)CC1 ; CS(=O)(=O)N1CCC(Nc2ncc3ccc(=O)n(CCCS(=O)(=O)n4cnc(C5CC5)n4)c3n2)CC1 ; CS(=O)(=O)N1CCC(Nc2ncc3ccc(=O)n(CCCS(=O)(=O)n4cnc(-c5ccccc5)n4)c3n2)CC1] 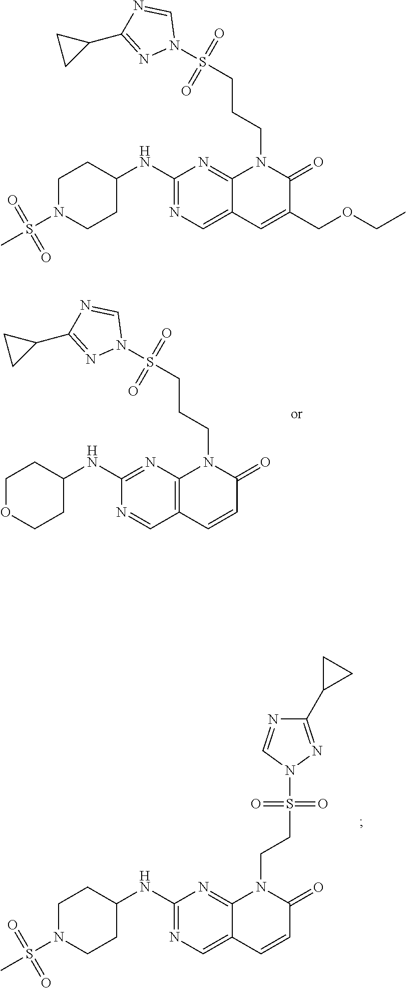
or a pharmaceutically acceptable salt thereof.

32. The pharmaceutically acceptable compound of claim 1 of structure: 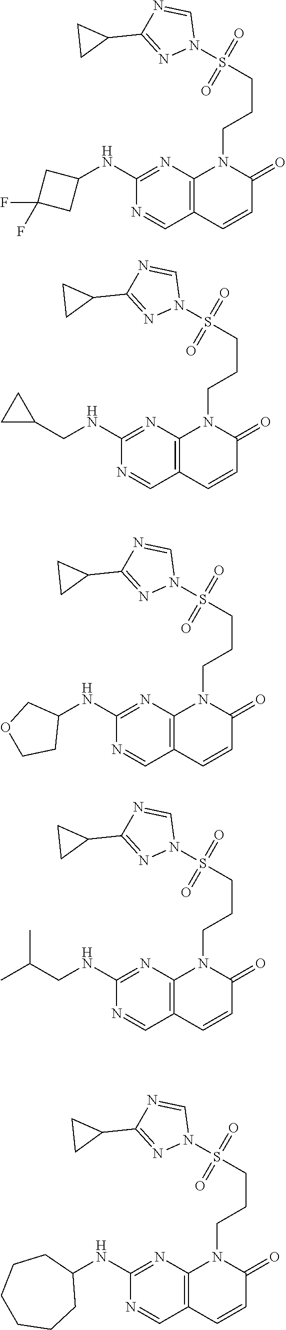 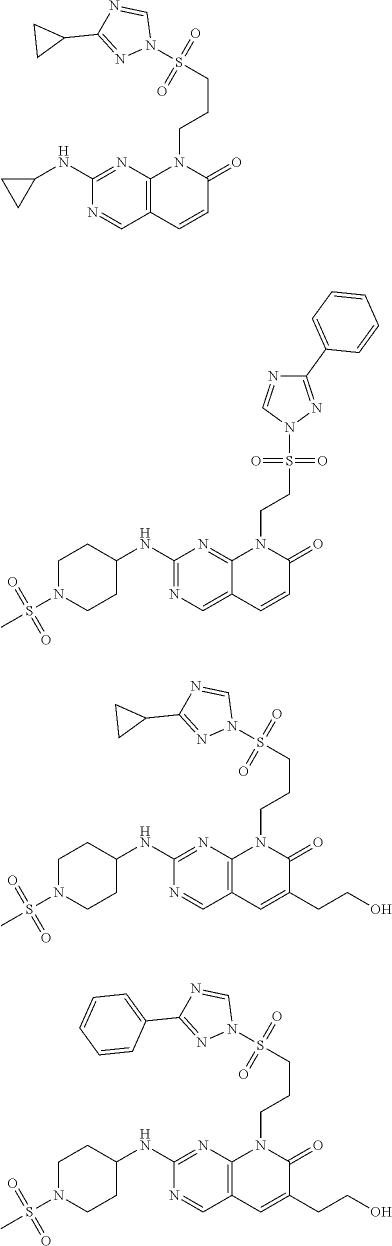 [SMILES: O=c1ccc2cnc(NC3CC3)nc2n1CCCS(=O)(=O)n1cnc(C2CC2)n1 ; CS(=O)(=O)N1CCC(Nc2ncc3ccc(=O)n(CCS(=O)(=O)n4cnc(-c5ccccc5)n4)c3n2)CC1 ; CS(=O)(=O)N1CCC(Nc2ncc3cc(CCO)c(=O)n(CCCS(=O)(=O)n4cnc(C5CC5)n4)c3n2)CC1 ; CS(=O)(=O)N1CCC(Nc2ncc3cc(CCO)c(=O)n(CCCS(=O)(=O)n4cnc(-c5ccccc5)n4)c3n2)CC1] 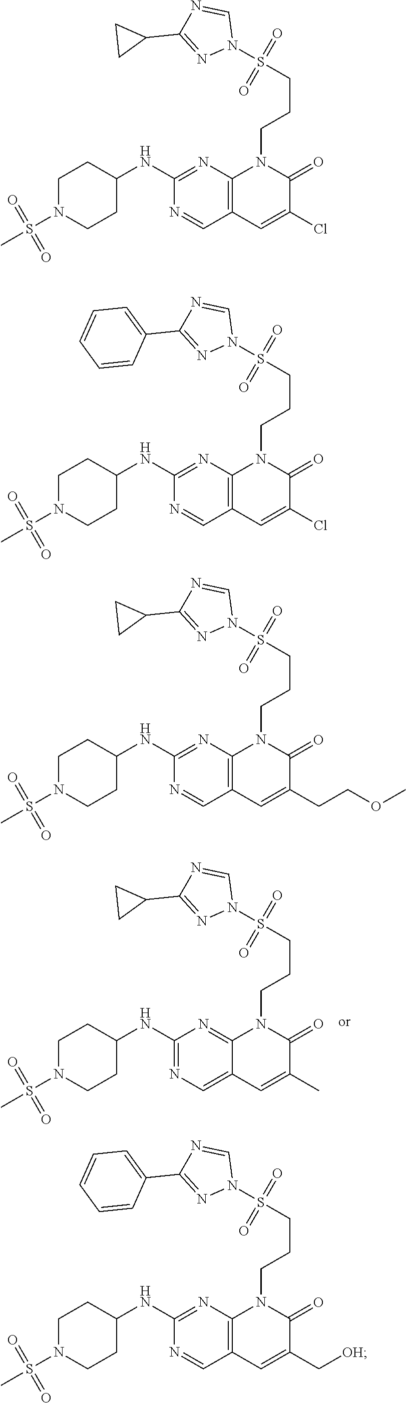
or a pharmaceutically acceptable salt thereof.

33. The pharmaceutically acceptable compound of claim 1 of structure: 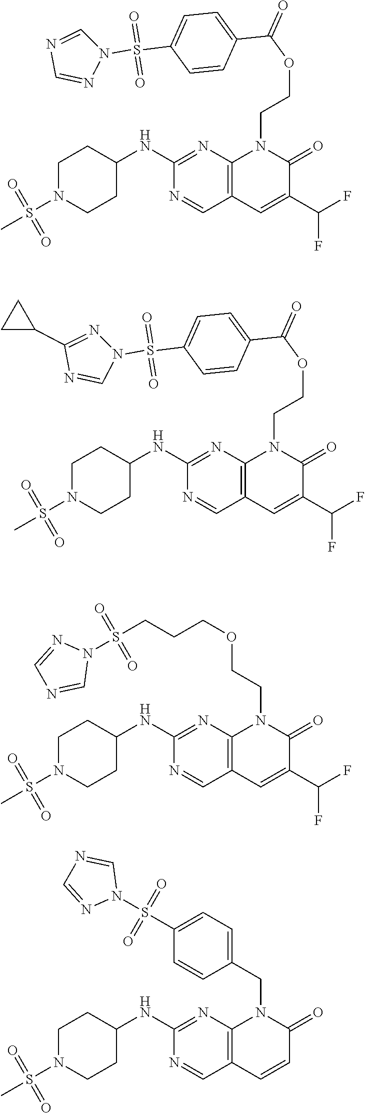 [SMILES: CS(=O)(=O)N1CCC(Nc2ncc3cc(C(F)F)c(=O)n(CCOC(=O)c4ccc(S(=O)(=O)n5cncn5)cc4)c3n2)CC1 ; CS(=O)(=O)N1CCC(Nc2ncc3cc(C(F)F)c(=O)n(CCOC(=O)c4ccc(S(=O)(=O)n5cnc(C6CC6)n5)cc4)c3n2)CC1 ; CS(=O)(=O)N1CCC(Nc2ncc3cc(C(F)F)c(=O)n(CCOCCCS(=O)(=O)n4cncn4)c3n2)CC1 ; CS(=O)(=O)N1CCC(Nc2ncc3ccc(=O)n(Cc4ccc(S(=O)(=O)n5cncn5)cc4)c3n2)CC1] 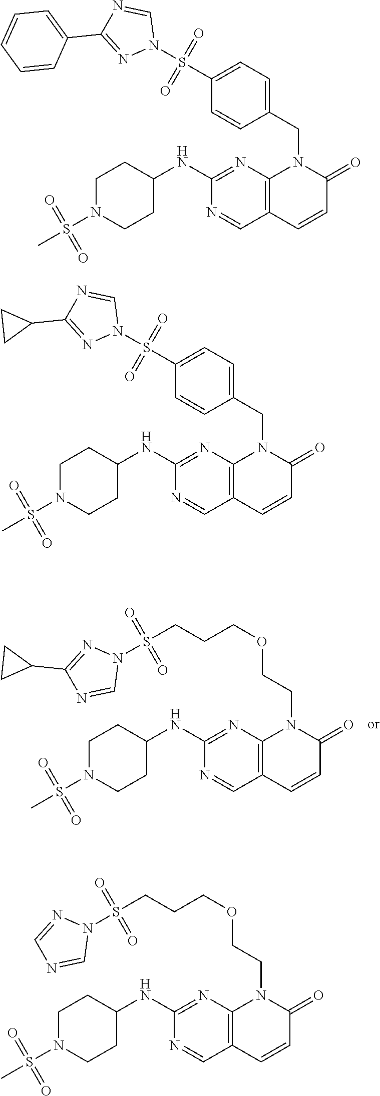
or a pharmaceutically acceptable salt thereof.

34. The pharmaceutically acceptable compound of claim 1 of structure: 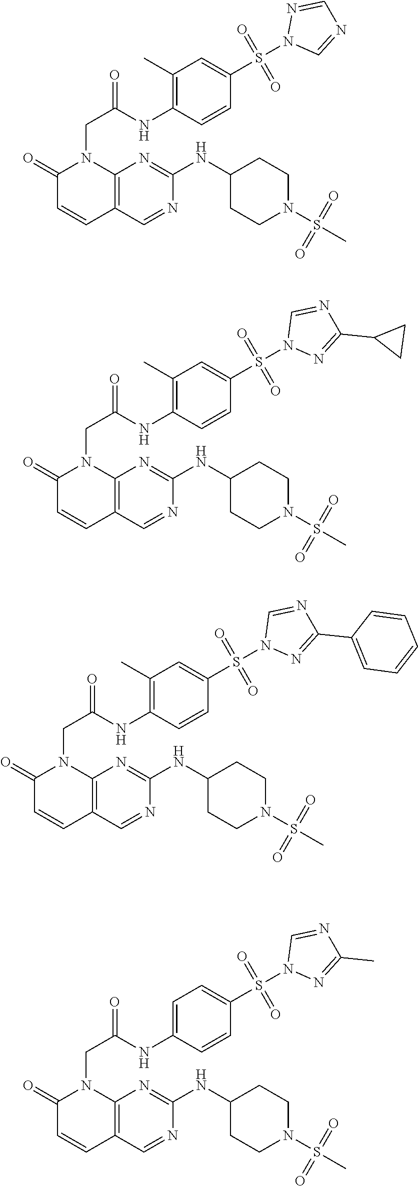 [SMILES: Cc1cc(S(=O)(=O)n2cncn2)ccc1NC(=O)Cn1c(=O)ccc2cnc(NC3CCN(S(C)(=O)=O)CC3)nc21 ; Cc1cc(S(=O)(=O)n2cnc(C3CC3)n2)ccc1NC(=O)Cn1c(=O)ccc2cnc(NC3CCN(S(C)(=O)=O)CC3)nc21 ; Cc1cc(S(=O)(=O)n2cnc(-c3ccccc3)n2)ccc1NC(=O)Cn1c(=O)ccc2cnc(NC3CCN(S(C)(=O)=O)CC3)nc21 ; Cc1ncn(S(=O)(=O)c2ccc(NC(=O)Cn3c(=O)ccc4cnc(NC5CCN(S(C)(=O)=O)CC5)nc43)cc2)n1] 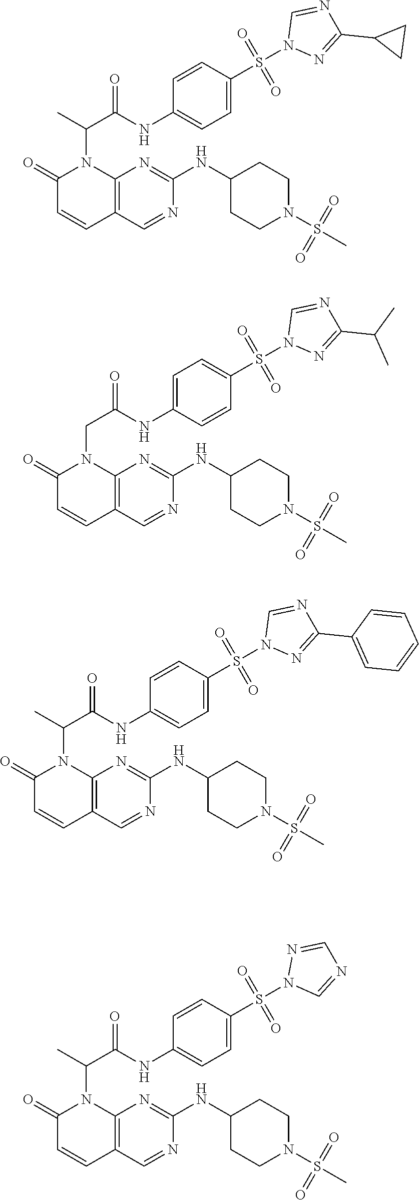 [SMILES: CC(C(=O)Nc1ccc(S(=O)(=O)n2cnc(C3CC3)n2)cc1)n1c(=O)ccc2cnc(NC3CCN(S(C)(=O)=O)CC3)nc21 ; CC(C)c1ncn(S(=O)(=O)c2ccc(NC(=O)Cn3c(=O)ccc4cnc(NC5CCN(S(C)(=O)=O)CC5)nc43)cc2)n1 ; CC(C(=O)Nc1ccc(S(=O)(=O)n2cnc(-c3ccccc3)n2)cc1)n1c(=O)ccc2cnc(NC3CCN(S(C)(=O)=O)CC3)nc21 ; CC(C(=O)Nc1ccc(S(=O)(=O)n2cncn2)cc1)n1c(=O)ccc2cnc(NC3CCN(S(C)(=O)=O)CC3)nc21] 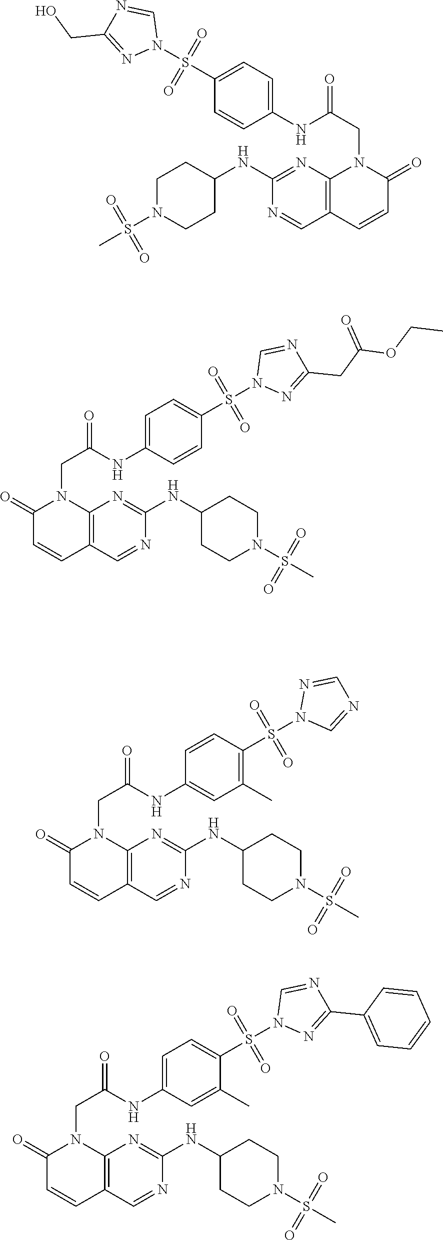 [SMILES: CS(=O)(=O)N1CCC(Nc2ncc3ccc(=O)n(CC(=O)Nc4ccc(S(=O)(=O)n5cnc(CO)n5)cc4)c3n2)CC1 ; CCOC(=O)Cc1ncn(S(=O)(=O)c2ccc(NC(=O)Cn3c(=O)ccc4cnc(NC5CCN(S(C)(=O)=O)CC5)nc43)cc2)n1 ; Cc1cc(NC(=O)Cn2c(=O)ccc3cnc(NC4CCN(S(C)(=O)=O)CC4)nc32)ccc1S(=O)(=O)n1cncn1 ; Cc1cc(NC(=O)Cn2c(=O)ccc3cnc(NC4CCN(S(C)(=O)=O)CC4)nc32)ccc1S(=O)(=O)n1cnc(-c2ccccc2)n1] 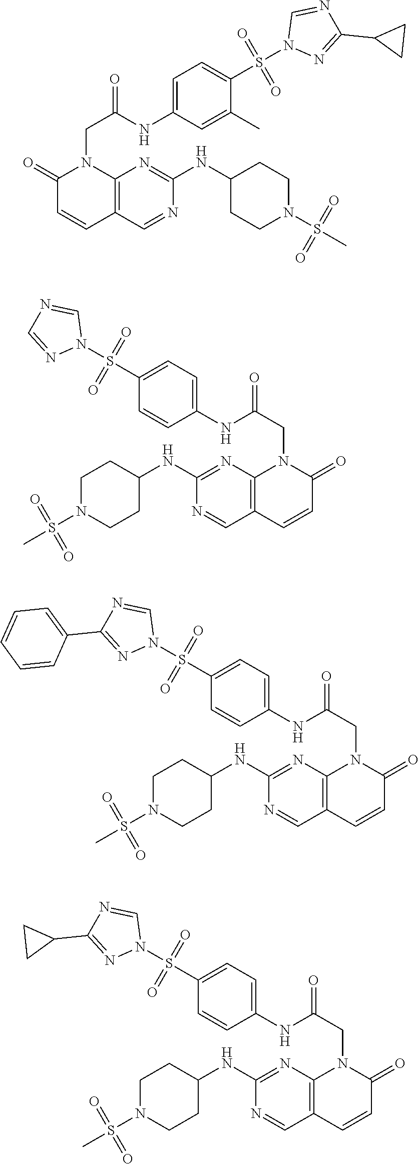 [SMILES: Cc1cc(NC(=O)Cn2c(=O)ccc3cnc(NC4CCN(S(C)(=O)=O)CC4)nc32)ccc1S(=O)(=O)n1cnc(C2CC2)n1 ; CS(=O)(=O)N1CCC(Nc2ncc3ccc(=O)n(CC(=O)Nc4ccc(S(=O)(=O)n5cncn5)cc4)c3n2)CC1 ; CS(=O)(=O)N1CCC(Nc2ncc3ccc(=O)n(CC(=O)Nc4ccc(S(=O)(=O)n5cnc(-c6ccccc6)n5)cc4)c3n2)CC1 ; CS(=O)(=O)N1CCC(Nc2ncc3ccc(=O)n(CC(=O)Nc4ccc(S(=O)(=O)n5cnc(C6CC6)n5)cc4)c3n2)CC1] 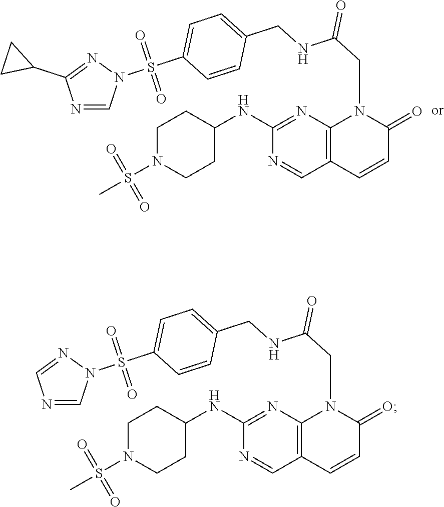 [SMILES: CS(=O)(=O)N1CCC(Nc2ncc3ccc(=O)n(CC(=O)NCc4ccc(S(=O)(=O)n5cnc(C6CC6)n5)cc4)c3n2)CC1 ; CS(=O)(=O)N1CCC(Nc2ncc3ccc(=O)n(CC(=O)NCc4ccc(S(=O)(=O)n5cncn5)cc4)c3n2)CC1]
or a pharmaceutically acceptable salt thereof.

35. The pharmaceutically acceptable compound of claim 1 of structure: 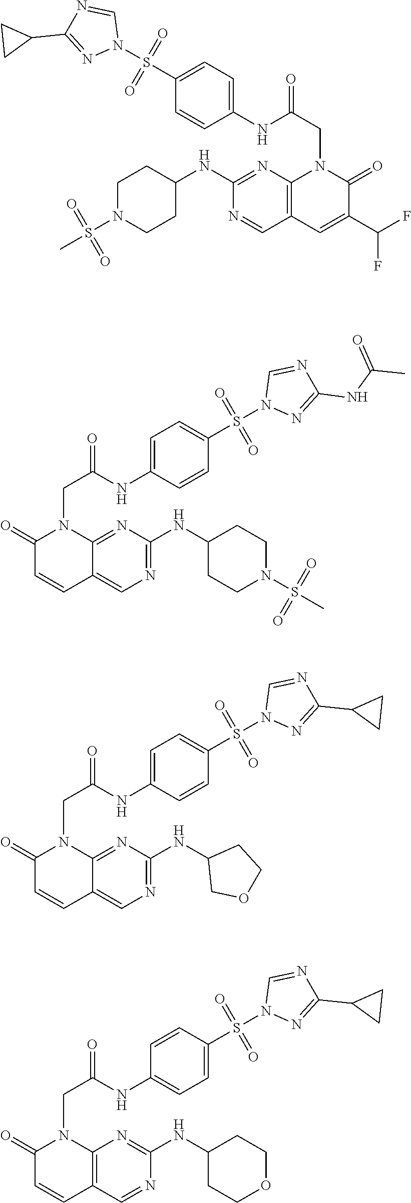 [SMILES: CS(=O)(=O)N1CCC(Nc2ncc3cc(C(F)F)c(=O)n(CC(=O)Nc4ccc(S(=O)(=O)n5cnc(C6CC6)n5)cc4)c3n2)CC1 ; CC(=O)Nc1ncn(S(=O)(=O)c2ccc(NC(=O)Cn3c(=O)ccc4cnc(NC5CCN(S(C)(=O)=O)CC5)nc43)cc2)n1 ; O=C(Cn1c(=O)ccc2cnc(NC3CCOC3)nc21)Nc1ccc(S(=O)(=O)n2cnc(C3CC3)n2)cc1 ; O=C(Cn1c(=O)ccc2cnc(NC3CCOCC3)nc21)Nc1ccc(S(=O)(=O)n2cnc(C3CC3)n2)cc1] 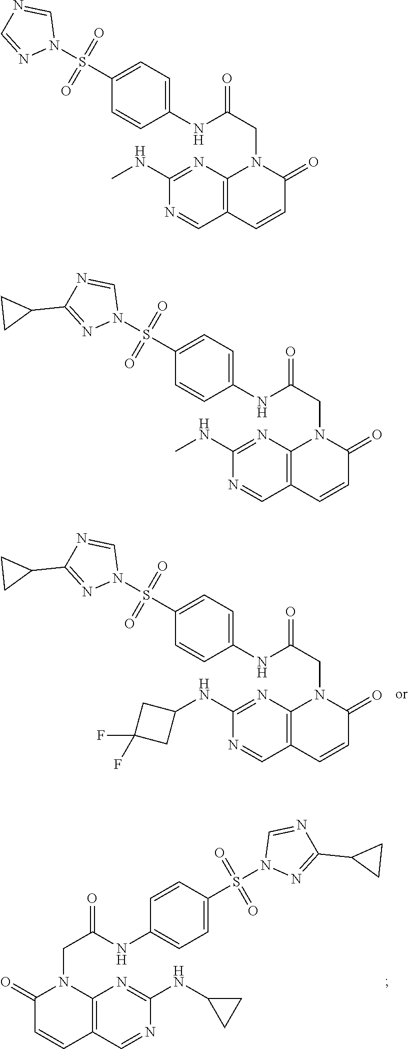 [SMILES: CNc1ncc2ccc(=O)n(CC(=O)Nc3ccc(S(=O)(=O)n4cncn4)cc3)c2n1 ; CNc1ncc2ccc(=O)n(CC(=O)Nc3ccc(S(=O)(=O)n4cnc(C5CC5)n4)cc3)c2n1 ; O=C(Cn1c(=O)ccc2cnc(NC3CC(F)(F)C3)nc21)Nc1ccc(S(=O)(=O)n2cnc(C3CC3)n2)cc1 ; O=C(Cn1c(=O)ccc2cnc(NC3CC3)nc21)Nc1ccc(S(=O)(=O)n2cnc(C3CC3)n2)cc1]
or a pharmaceutically acceptable salt thereof.

36. A pharmaceutical composition comprising a pharmaceutically acceptable compound of claim 1 or a pharmaceutically acceptable salt thereof and a pharmaceutically acceptable excipient.

37. The pharmaceutical composition of claim 36, wherein the composition is suitable for oral delivery.

38. The pharmaceutical composition of claim 36, wherein the pharmaceutically acceptable compound is of Formula: 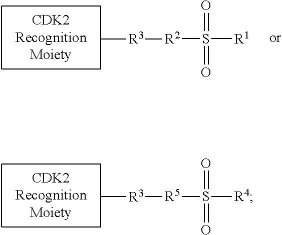
or a pharmaceutically acceptable salt thereof.

39. The pharmaceutical composition of claim 36, wherein the pharmaceutically acceptable compound is Formula: 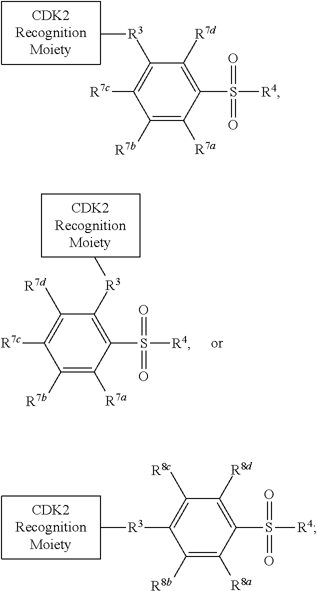
or a pharmaceutically acceptable salt thereof.

40. The pharmaceutical composition of claim 36, wherein the pharmaceutically acceptable compound is of Formula: 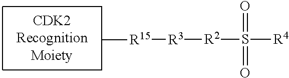
or a pharmaceutically acceptable salt thereof.

41. The pharmaceutical composition of claim 36, wherein the pharmaceutically acceptable compound is of Formula: 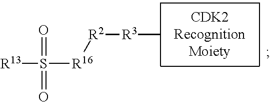
or a pharmaceutically acceptable salt thereof.

42. A method of treating a disorder mediated by CDK2 comprising administering an effective amount of a pharmaceutically acceptable compound of claim 1 or a pharmaceutically acceptable salt thereof or a pharmaceutical composition thereof to a human in need thereof.

43. The method of claim 42, wherein the disorder is a cancer.

44. The method of claim 43, wherein the cancer is a solid cancer.

45. The method of claim 43, wherein the cancer is a hematological cancer.

46. The method of claim 43, wherein the cancer is breast cancer.

47. The method of claim 46, wherein the breast cancer is triple negative breast cancer.

48. The method of claim 46, wherein the breast cancer is hormone receptor positive HER2 negative breast cancer.

49. The method of claim 43, wherein the cancer is ovarian cancer.

50. The method of claim 43, wherein the cancer is metastatic.

51. The method of claim 43, wherein the cancer is relapsed.

52. The method of claim 43, wherein the cancer is refractory.

53. The method of claim 43, wherein the pharmaceutically acceptable compound is of Formula: 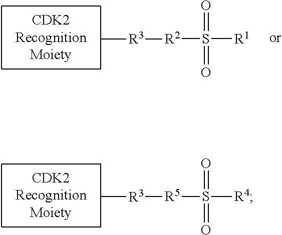
or a pharmaceutically acceptable salt thereof.

54. The method of claim 43, wherein the pharmaceutically acceptable compound is Formula: 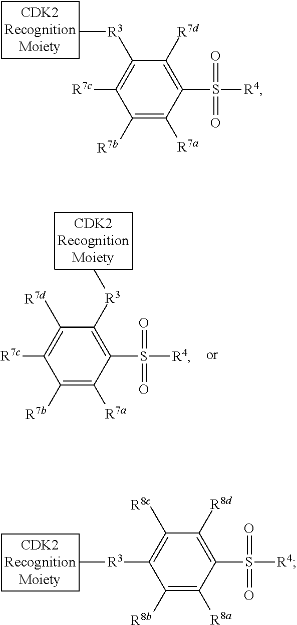
or a pharmaceutically acceptable salt thereof.

55. The method of claim 43, wherein the pharmaceutically acceptable compound is of Formula: 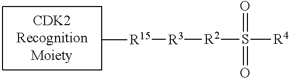
or a pharmaceutically acceptable salt thereof.

56. The method of claim 43, wherein the pharmaceutically acceptable compound is of Formula: 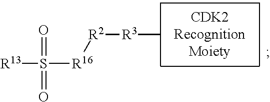
or a pharmaceutically acceptable salt thereof.
